(19) United States
(12) Patent Application Publication
(10) Pub. No.: US 2004/0053999 A1
(43) Pub. Date: Mar. 18, 2004

(54) Novel compounds and methods for synthesis and therapy

(76) Inventors: Norbert W. Bischofberger, San Carlos, CA (US); Terrence C. Dahl, Sunnyvale, CA (US); Michael J. M. Hitchcock, San Mateo, CA (US); Choung U. Kim, San Carlos, CA (US); Willard Lew, San Mateo, CA (US); Hongtao Liu, Foster City, CA (US); Roger G. Mills, Menlo Park, CA (US); Matthew A. Williams, Foster City, CA (US)

Correspondence Address: GILEAD SCIENCES INC, 333 LAKESIDE DR, FOSTER CITY, CA, 94404, US

(21) Appl. No.: 10/628,773

(22) Filed: Jul. 28, 2003

Related U.S. Application Data
(63) Continuation of application No. 09/153,964, filed on Sep. 16, 1998.
(60) Provisional application No. 60/060,195, filed on Sep. 26, 1997.
(60) Provisional application No. 60/059,308, filed on Sep. 17, 1997.

(51) Int. Cl. A61K 31/655; A61K 31/275; A61K 31/215; A61K 31/195

(52) U.S. Cl. 514/519; 514/529; 514/563; 514/151

(57) ABSTRACT

Novel compounds are described. The compounds generally comprise an acidic group, a basic group, a substituted amino or N-acyl and a group having an optionally hydroxylated alkane moiety. Pharmaceutical compositions comprising the inhibitors of the invention are also described. Methods of inhibiting neuraminidase in samples suspected of containing neuraminidase are also described. Antigenic materials, polymers, antibodies, conjugates of the compounds of the invention with labels, and assay methods for detecting neuraminidase activity are also described.

### CROSS REFERENCE TO RELATED APPLICATION

This application is a continuation of U.S. Ser. No. 09/153,964, filed Sep. 16, 1998, which claims the benefit of priority of U.S. Provisional Application Serial No. 60/060,195, filed Sep. 26, 1997.

This application is also based on U.S. Patent Application Serial No. 08/938,644, filed Sep. 26, 1997, and U.S. Provisional Application Serial No. 60/059,308, filed Sep. 17, 1997.

This application is also related to U.S. patent application Ser. No. 08/653,034, filed Mar. 24, 1996, which was a continuation-in-part application of U.S. patent application Ser. No. 08/606,624, filed Feb. 26, 1996, which was a continuation-in-part application of U.S. patent application Ser. No. 08/580,567, filed Dec. 29, 1995, which was a continuation-in-part application of U.S. patent application Ser. No. 08/476,946, filed Jun. 6, 1995, which was a continuation-in-part application of U.S. patent application Ser. No. 08/395,245, filed Feb. 27, 1995, all of which are incorporated herein by reference in their entirety. This application is related to U.S. patent application Ser. No. 08/917,640, filed Aug. 22, 1997, which describes methods of making carbocyclic compounds in particular methods of making GS 4104, phosphate salt, and is incorporated by reference in its entirety.

### FIELD OF THE INVENTION

Neuraminidase (also known as sialidase, acylneuraminyl hydrolase, and EC 3.2.1.18) is an enzyme common among animals and a number of microorganisms. It is a glycohydrolase that cleaves terminal alpha-ketosidically linked sialic acids from glycoproteins, glycolipids and oligiosaccharides. Many of the microorganisms containing neuraminidase are pathogenic to man and other animals including fowl, horses, swine and seals. These pathogenic organisms include influenza virus.

Neuraminidase has been implicated in the pathogenicity of influenza viruses. It is thought to help the elution of newly synthesized virons from infected cells and assist in the movement of the virus (through its hydrolase activity) through the mucus of the respiratory tract.

### BRIEF DESCRIPTION OF RELATED ART

von Itzstein, M. et al.; “Nature”, 363(6428):418-423 (1993), discloses the rational design of sialidase-based inhibitors of influenza virus replication.

Colman, P. M. et al.; International Patent Publication No. WO 92/06691 (Int. App. No. PCT/AU90/00501, publication date Apr. 30, 1992), von Itzstein, L. M. et al.; European Patent Publication No. 0 539 204 A1 (EP App. No. 92309684.6, publication date Apr. 28, 1993), and von Itzstein, L. M. et al.; International Publication No. WO 91/16320 (Int. App. No. PCT/AU91/00161, publication date Oct. 31, 1991) disclose compounds that bind neuraminidase and are asserted to exhibited antiviral activity in vivo.

### OBJECTS OF THE INVENTION

A principal object of the invention is inhibition of viruses, in particular influenza viruses. In particular, an object is inhibition of glycolytic enzymes such as neuraminidase, in particular the selective inhibition of viral or bacterial neuraminidases.

An additional object of the invention is to provide neuraminidase inhibitors that have a retarded rate of urinary excretion, that enter into nasal or pulmonary secretions from the systemic circulation, that have sufficient oral bioavailability to be therapeutically effective, that possess elevated potency, that exhibit clinically acceptable toxicity profiles and have other desirable pharmacologic properties.

Another object is to provide improved and less costly methods for synthesis of neuraminidase inhibitors.

A still further object is to provide improved methods for administration of known and novel neuraminidase inhibitors.

An additional object is to provide compositions useful in preparing polymers, surfactants or immunogens and for use in other industrial processes and articles

These and other objects will be readily apparent to the ordinary artisan from consideration of the invention as a whole.

### SUMMARY OF THE INVENTION

Compounds, or compositions having formula (I) or (II) are provided herein:

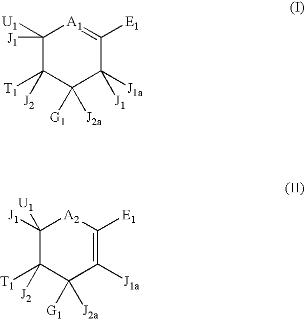

wherein

A₁ is —C(J₁)═, —N═ or —N(O)═;

A₂ is —C(J₁)₂—, —N(J₁)—, —N(O)(J₁)—, —S—, —S(O)—, —S(O)₂— or —O—;

E₁ is —(CR₁R₁)ₘ₁W₁;

G₁ is N₃, —CN, —OH, —OR₆ₐ, —NO₂, or —(CR₁R₁)ₘ₁W₂;

T₁ is —NR₁W₃, H, —R₃, —R₅, a heterocycle, or is taken together with U₁ or G₁ to form a group having the structure

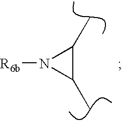

U₁ is H, —R₃ or —X₁W₆;

J₁ and J₁ₐ are independently R₁, Br, Cl, F, I, CN, NO₂ or N₃;

J₂ and J₂ₐ are independently H or R₁;

R₁ is independently H or alkyl of 1 to 12 carbon atoms;

R₂ is independently R₃ or R₄ wherein each R₄ is independently substituted with 0 to 3 R₃ groups;

R₃ is independently F, Cl, Br, I, —CN, N₃, —NO₂, OR₆ₐ, —OR₁, —N(R₁)₂, —N(R₁)(R_{6b}), —N(R_{6b})₂, —SR₁, SR₆ₐ, —S(O)R₁, —S(O)₂R₁, —S(O)OR₁, S(O)OR₆ₐ, —S(O)₂OR₁, S(O)₂OR₆ₐ, —C(O)OR₁, —C(O)R_{6c}, —C(O)OR₆ₐ, —OC(O)R₁, —N(R₁)(C(O)R₁), —N(R_{6b})(C(O)R₁), —N(R₁)(C(O)OR₁), —N(R_{6b})(C(O)OR₁), —C(O)N(R₁)₂, —C(O)N(R_{6b})(R₁), —C(O)N(R_{6b})₂, —C(NR₁)(N(R₁)₂), —C(N(R_{6b}))(N(R₁)₂), —C(N(R₁))(N(R₁)(R_{6b})), —C(N(R_{6b}))(N(R₁)(R_{6b})), —C(N(R₁))(N(R_{6b})₂), —C(N(R_{6b}))(N(R_{6b})₂), —N(R₁)C(N(R₁))(N(R₁)₂), —N(R₁)C(N(R₁))(N(R₁)(R_{6b})), —N(R₁)C(N(R_{6b}))(N(R₁)₂), —N(R_{6b})C(N(R₁))(N(R₁)₂), —N(R_{6b})C(N(R_{6b}))(N(R₁)₂), —N(R_{6b})C(N(R₁))(N(R₁)(R_{6b})), —N(R₁)C(N(R_{6b}))(N(R₁)(R_{6b})), —N(R₁)C(N(R₁))(N(R_{6b})₂), —N(R_{6b})C(N(R_{6b}))(N(R₁)(R_{6b})), —N(R_{6b})C(N(R₁))(N(R_{6b})₂), —N(R₁)C(N(R_{6b}))(N(R_{6b})₂), —N(R_{6b})C(N(R_{6b}))(N(R_{6b})₂), ═O, ═S, ═N(R₁), ═N(R_{6b}) or W₅;

R₄ is independently alkyl of 1 to 12 carbon atoms, alkenyl of 2 to 12 carbon atoms, or alkynyl of 2 to 12 carbon atoms;

R₅ is independently R₄ wherein each R₄ is substituted with 0 to 3 R₃ groups;

R₅ₐ is independently alkylene of 1 to 12 carbon atoms, alkenylene of 2 to 12 carbon atoms, or alkynylene of 2-12 carbon atoms any one of which alkylene, alkenylene or alkynylene is substituted with 0-3 R₃ groups;

R₆ₐ is independently H or an ether- or ester-forming group;

R_{6b} is independently H, a protecting group for amino or the residue of a carboxyl-containing compound;

R_{6c} is independently H or the residue of an amino-containing compound;

W₁ is a group comprising an acidic hydrogen, a protected acidic group, or an R_{6c} amide of the group comprising an acidic hydrogen;

W₂ is a group comprising a basic heteroatom or a protected basic heteroatom, or an R_{6b} amide of the basic heteroatom or a group derivatizable to a basic heteroatom;

W₃ is W₄ or W₅;

W₄ is R₅ or —C(O)R₅, —C(O)W₅, —SO₂R₅, or —SO₂W₅;

W₅ is carbocycle or heterocycle wherein W₅ is independently substituted with 0 to 3 R₂ groups;

W₆ is —R₅, —W₅, —R₅ₐW₅, —C(O)OR₆ₐ, —C(O)R_{6c}, —C(O)N(R_{6b})₂, —C(NR_{6b})(N(R_{6b})₂), —C(NR_{6b})(N(H)(R_{6b})), —C(N(H)(N(R_{6b})₂), —C(S)N(R_{6b})₂, or —C(O)R₂;

X₁ is a bond, —O—, —N(H)—, —N(W₆)—, —N(OH)—, —N(OW₆)—, —N(NH₂)—, —N(N(H)(W₆))—, —N(N(W₆)₂)—, —N(H)N(W₆)—, —S—, —SO—, or —SO₂—; and

each m₁ is independently an integer from 0 to 2; provided, however, that compounds are excluded that are described in WO 91/16320 at page 3, line 23 to page 5, line 6, which appear to include compounds wherein:

(a) A₁ is —CH═ or —N═ and A₂ is —CH₂—;

(b) E₁ is COOH, P(O)(OH)₂, SOOH, SO₃H, or tetrazol;

(c) G₁ is CN, N(H)R₂₀, N₃, SR₂₀, OR₂₀, guanidino, —N(H)CN

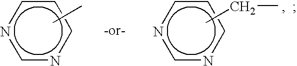

(d) T₁ is —NHR₂₀;

(e) R₂₀ is H; an acyl group having 1 to 4 carbon atoms; a linear or cyclic alkyl group having 1 to 6 carbon atoms, or a halogen-substituted analogue thereof; an allyl group or an unsubstituted aryl group or an aryl substituted by a halogen, an OH group, an NO₂ group, an NH₂ group or a COOH group;

(f) J₁ is H and J₁ₐ is H, F Cl, Br or CN;

(g) J₂ is H and J₂ₐ is H, CN or N₃;

(h) U₁ is CH₂YR₂₀ₐ, CHYR₂₀ₐCH₂YR₂₀ₐ or CHYR₂₀ₐCHYR₂₀ₐCH₂YR₂₀ₐ;

(i) R₂₀ₐ is H or acyl having 1 to 4 carbon atoms;

(j) Y is O, S, H or NH;

(k) 0 to 2 YR₂₀ₐ are H, and

(l) successive Y moieties in a U₁ group are the same or different, and when Y is H then R₂₀ₐ is a covalent bond, and provided that if G₁ is N₃ then U₁ is not —CH₂OCH₂Ph. and the pharmaceutically acceptable salts and solvates thereof;

and the salts, solvates, resolved enantiomers and purified diastereomers thereof.

Also excluded herein are compounds described in WO 92/06691 at Page 9, Line 26, to Page 11, Line 5, which appear to include compounds of the formula II wherein:

(a) A₂ is O;

(b) E₁ is COOH, P(O)(OH)₂, NO₂, SOOH, SO₃H, tetrazole, CH₂CHO, CHO, CH(CHO)₂ or where E₁ is COOH, P(O)(OH)₂, SOOH or SO₃H, an ethyl, methyl or pivaloyl ester thereof;

(c) G₁ is hydrogen, N(R^{20a})₂, SR^{20a} or OR^{20a};

(d) T₁ is —NHC(O)R^{20b}, where R_{20b} is an unsubstituted or halogen-substituted linear or cyclic alkyl group of 1 to 6 carbon atoms, or SR^{20a}, OR^{20a}, COOH or alkyl/aryl ester thereof, NO₂, C(R^{20a})₃, CH₂COOH or alkyl/aryl ester thereof, CH₂NO₂ or CH₂NHR^{20b};

(e) R^{20a} is hydrogen; an acyl group having 1 to 4 carbon atoms; a linear or cyclic alkyl group having 1 to 6 carbon atoms, or a halogen-substituted analogue thereof; or an unsubstituted aryl group or an aryl substituted by a halogen, an allyl group, an OH group, an NO₂ group, an NH₂ group or a COOH group;

(f) J₁ is H and J₁ₐ is H, OR^{20a}, F, Cl, Br, CN, NHR^{20a}, SR^{20a} or CH₂X wherein X is NHR^{20a}, halogen or OR^{20a};

(g) J₂ is H or J₂ₐ is hydrogen, N(R^{20a})₂, SR^{20a} or OR^{20a};

(h) U₁ is CH₂YR^{20a}, CHYR²⁰CH₂YR^{20a} or CHYR^{20a}CHYR^{20a}CH₂YR^{20a} where Y is O, S or H, and successive Y moieties in U₁ are the same or different and R^{20a} represents a covalent bond when Y is hydrogen and

and pharmacologically acceptable salts or derivatives thereof. Another embodiment of the invention is directed to compounds of the formula:

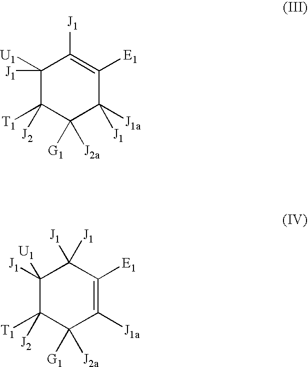

wherein

E₁ is —(CR₁R₁)ₘ₁W₁;

G₁ is N₃, —CN, —OH, —OR₆ₐ, —NO₂, or —(CR₁R₁)ₘ₁W₂;

T₁ is —NR₁W₃, a heterocycle, or is taken together with U₁ or G₁ to form a group having the structure

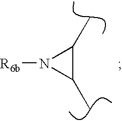

U₁ is H or —X₁W₆ and, if —X₁W₆, then U₁ is a branched chain;

J₁ and J₁ₐ are independently R₁, Br, Cl, F, I, CN, NO₂ or N₃;

J₂ and J₂ₐ are independently H or R₁;

R₁ is independently H or alkyl of 1 to 12 carbon atoms;

R₂ is independently R₃ or R₄ wherein each R₄ is independently substituted with 0 to 3 R₃ groups;

R₃ is independently F, Cl, Br, I, —CN, N₃, —NO₂, —OR₆ₐ, —OR₁, —N(R₁)₂, —N(R₁)(R_{6b}), —N(R_{6b})₂, —SR₁, —SR₆ₐ, —S(O)R₁, —S(O)₂R₁, —S(O)OR₁, —S(O)OR₆ₐ, —S(O)₂OR₁, —S(O)₂OR₆ₐ, —C(O)OR₁, —C(O)R_{6c}, —C(O)OR₆ₐ, —OC(O)R₁, —N(R₁)(C(O)R₁), —N(R_{6b})(C(O)R₁), —N(R₁)(C(O)OR₁), —N(R_{6b})(C(O)OR₁), —C(O)N(R₁)₂, —C(O)N(R_{6b})(R₁), —C(O)N(R_{6b})₂, —C(NR₁)(N(R₁)₂), —C(N(R_{6b}))(N(R₁)₂), —C(N(R₁))(N(R₁)(R_{6b})), —C(N(R_{6b}))(N(R₁)(R_{6b})), —C(N(R₁))(N(R_{6b})₂), —C(N(R_{6b}))(N(R_{6b})₂), —N(R₁)C(N(R₁))(N(R₁)₂), —N(R₁)C(N(R₁))(N(R₁)(R_{6b})), —N(R₁)C(N(R_{6b}))(N(R₁)₂), —N(R_{6b})C(N(R₁))(N(R₁)₂), —N(R_{6b})C(N(R_{6b}))(N(RI)₂), —N(R_{6b})C(N(R₁))(N(R₁)(R_{6b})), —N(R₁)C(N(R_{6b}))(N(R₁)(R_{6b})), —N(R₁)C(N(R₁))(N(R_{6b})₂), —N(R_{6b})C(N(R_{6b}))(N(R₁)(R_{6b})), —N(R_{6b})C(N(R₁))(N(R_{6b})₂), —N(R₁)C(N(R_{6b}))(N(R_{6b})₂), —N(R_{6b})C(N(R_{6b}))(N(R_{6b})₂), ═O, ═S, ═N(R₁) or ═N(R_{6b});

R₄ is independently alkyl of 1 to 12 carbon atoms, alkenyl of 2 to 12 carbon atoms, or alkynyl of 2 to 12 carbon atoms;

R₅ is independently R₄ wherein each R₄ is substituted with 0 to 3 R₃ groups;

R₅ₐ is independently alkylene of 1 to 12 carbon atoms, alkenylene of 2 to 12 carbon atoms, or alkynylene of 2-12 carbon atoms any one of which alkylene, alkenylene or alkynylene is substituted with 0-3 R₃ groups;

R₆ₐ is independently H or an ether- or ester-forming group;

R_{6b} is independently H, a protecting group for amino or the residue of a carboxyl-containing compound;

R_{6c} is independently H or the residue of an amino-containing compound;

W₁ is a group comprising an acidic hydrogen, a protected acidic group, or an R_{6c} amide of the group comprising an acidic hydrogen;

W₂ is a group comprising a basic heteroatom or a protected basic heteroatom, or an R_{6b} amide of the basic heteroatom;

W₃ is W₄ or W₅;

W₄ is R₅ or —C(O)R₅, —C(O)W₅, —SO₂R₅, or —SO₂W₅;

W₅ is carbocycle or heterocycle wherein W₅ is independently substituted with 0 to 3 R₂ groups;

W₆ is —R₅, —W₅, —R₅ₐW₅, —C(O)OR₆ₐ, —C(O)R_{6c}, —C(O)N(R_{6b})₂, —C(NR_{6b})(N(R_{6b})₂), —C(S)N(R_{6b})₂, or —C(O)R₂;

X₁ is a bond, —O—, —N(H)—, —N(W₆)—, —N(OH)—, —N(OW₆)—, —N(NH₂)—, —N(N(H)(W₆))—, —N(N(W₆)₂)—, —N(H)N(W₆)—, —S—, —SO—, or —SO₂—; and

each m₁ is independently an integer from 0 to 2;

and the salts, solvates, resolved enantiomers and purified diastereomers thereof.

Another embodiment of the invention is directed to compounds of the formula:

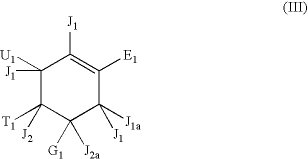

wherein

E₁ is —(CR₁R₁)ₘ₁W₁;

G₁ is N₃, —CN, —OH, —OR₆ₐ, —NO₂, or —(CR₁R₁)ₘ₁W₂;

T₁ is —NR₁W₃, a heterocycle, or is taken together with U₁ or G₁ to form a group having the structure

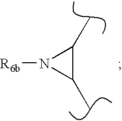

U₁ is H or —X₁W₆;

J₁ and J₁ₐ are independently R₁, Br, Cl, F, I, CN, NO₂ or N₃;

J₂ and J₂ₐ are independently H or R₁;

R₁ is independently H or alkyl of 1 to 12 carbon atoms;

R₂ is independently R₃ or R₄ wherein each R₄ is independently substituted with 0 to 3 R₃ groups;

R₃ is independently F, Cl, Br, I, —CN, N₃, —NO₂, —OR₆ₐ, —OR₁, —N(R₁)₂, —N(R₁)(R_{6b}), —N(R_{6b})₂, —SR₁, —SR₆ₐ, —S(O)R₁, —S(O)₂R₁, —S(O)OR₁, —S(O)OR₆ₐ, —S(O)₂OR₁, —S(O)₂OR₆ₐ, —C(O)OR₁, —C(O)R_{6c}, —C(O)OR₆ₐ, —OC(O)R₁, —N(R₁)(C(O)R₁), —N(R_{6b})(C(O)R₁), —N(R₁)(C(O)OR₁), —N(R_{6b})(C(O)OR₁), —C(O)N(R₁)₂, —C(O)N(R_{6b})(R₁), —C(O)N(R_{6b})₂, —C(NR₁)(N(R₁)₂), —C(N(R_{6b}))(N(R₁)₂), —C(N(R₁))(N(R₁)(R_{6b})), —C(N(R_{6b}))(N(R₁)(R_{6b})), —C(N(R₁))(N(R_{6b})₂), —C(N(R_{6b}))(N(R_{6b})₂), —N(R₁)C(N(R₁))(N(R₁)₂), —N(R₁)C(N(R₁))(N(R₁)(R_{6b})), —N(R₁)C(N(R_{6b}))(N(R₁)₂), —N(R_{6b})C(N(R₁))(N(R₁)₂), —N(R_{6b})C(N(R_{6b}))(N(R₁)₂), —N(R_{6b})C(N(R₁))(N(R₁)(R_{6b})), —N(R₁)C(N(R_{6b}))(N(R₁)(R_{6b})), —N(R₁)C(N(R₁))(N(R_{6b})₂), —N(R_{6b})C(N(R_{6b}))(N(R₁)(R_{6b})), —N(R_{6b})C(N(R₁))(N(R_{6b})₂), —N(R₁)C(N(R_{6b}))(N(R_{6b})₂), —N(R_{6b})C(N(R_{6b}))(N(R_{6b})₂), ═O, ═S, ═N(R₁) or ═N(R_{6b});

R₄ is independently alkyl of 1 to 12 carbon atoms, alkenyl of 2 to 12 carbon atoms, or alkynyl of 2 to 12 carbon atoms;

R₅ is independently R₄ wherein each R₄ is substituted with 0 to 3 R₃ groups;

R₅ₐ is independently alkylene of 1 to 12 carbon atoms, alkenylene of 2 to 12 carbon atoms, or alkynylene of 2-12 carbon atoms any one of which alkylene, alkenylene or alkynylene is substituted with 0-3 R₃ groups;

R₆ₐ is independently H or an ether- or ester-forming group;

R_{6b} is independently H, a protecting group for amino or the residue of a carboxyl-containing compound;

R_{6c} is independently H or the residue of an amino-containing compound;

W₁ is a group comprising an acidic hydrogen, a protected acidic group, or an R_{6c} amide of the group comprising an acidic hydrogen;

W₂ is a group comprising a basic heteroatom or a protected basic heteroatom, or an R_{6b} amide of the basic heteroatom;

W₃ is W₄ or W₅;

W₄ is R₅ or —C(O)R₅, —C(O)W₅, —SO₂R₅, or —SO₂W₅;

W₅ is carbocycle or heterocycle wherein W₅ is independently substituted with 0 to3R₂ groups;

W₆ is —R₅, —W₅, —R₅ₐW₅, —C(O)OR₆ₐ, —C(O)R_{6c}, —C(O)N(R_{6b})₂, —C(NR_{6b})(N(R_{6b})₂), —C(S)N(R_{6b})₂, or —C(O)R₂;

X₁ is —O—, —N(H)—, —N(W₆)—, —N(OH)—, —N(OW₆)—, —N(NH₂)—, —N(N(H)(W₆))—, —N(N(W₆)₂)—, —N(H)N(W₆)—, —S—, —SO—, or —SO₂—; and

each m₁ is independently an integer from 0 to 2;

and the salts, solvates, resolved enantiomers and purified diastereomers thereof.

Another embodiment of the invention is directed to compounds of the formula:

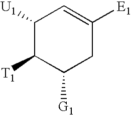

wherein:

E₁ is —CO₂R₁;

G₁ is —NH₂, —N(H)(R₅) or —N(H)(C(N(H))(NH₂));

T₁ is —N(H)(C(O)CH₃);

U₁ is —OR₆₀;

R₁ is H or an alkyl of 1, 2, 3, 4, 5, 6, 7, 8, 9, 10, 11, or 12 carbon atoms; and

R₆₀ is a branched alkyl of 3, 4, 5, 6, 7, 8, 9, 10, 11, or 12 carbon atoms;

and the salts, solvates, resolved enantiomers and purified diastereomers thereof.

Another embodiment of the invention is directed to compounds of formulas (VII) or (VIII):

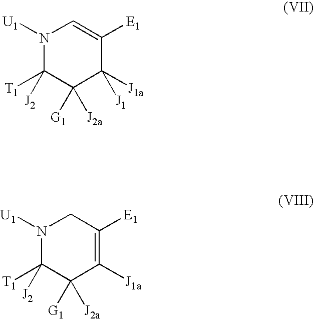

wherein

E₁ is —(CR₁R₁)ₘ₁W₁;

G₁ is N₃, —CN, —OH, —OR₆ₐ, —NO₂, or —(CR₁R₁)ₘ₁W₂;

T₁ is —NR₁W₃, a heterocycle, or is taken together with G₁ to form a group having the structure

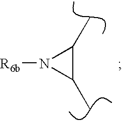

U1 is —X₁W₆;

J₁ and J₁ₐ are independently R₁, Br, Cl, F, I, CN, NO₂ or N₃;

J₂ and J₂ₐ are independently H or R₁;

R₁ is independently H or alkyl of 1 to 12 carbon atoms;

R₂ is independently R₃ or R₄ wherein each R₄ is independently substituted with 0 to 3 R₃ groups;

R₃ is independently F, Cl, Br, I, —CN, N₃, —NO₂, OR₆ₐ, —OR₁, —N(R₁)₂, —N(R₁)(R_{6b}), —N(R_{6b})₂, —SR₁, —SR₆ₐ, —S(O)R₁, —S(O)₂R₁, —S(O)OR₁, S(O)OR₆ₐ, —S(O)₂OR₁, —S(O)₂OR₆ₐ, —C(O)OR₁, —C(O)R_{6c}, —C(O)OR₆ₐ, —OC(O)R₁, —N(R₁)(C(O)R₁), —N(R_{6b})(C(O)R₁), —N(R₁)(C(O)OR₁), —N(R_{6b})(C(O)OR₁), —C(O)N(R₁)₂, —C(O)N(R_{6b})(R₁), —C(O)N(R_{6b})₂, —C(NR₁)(N(R₁)₂), —C(N(R_{6b}))(N(R₁)₂), —C(N(R₁))(N(R₁)(R_{6b})), —C(N(R_{6b}))(N(R₁)(R_{6b})), —C(N(R₁))(N(R_{6b})₂), —C(N(R_{6b}))(N(R_{6b})₂), —N(R₁)C(N(R₁))(N(R₁)₂), —N(R₁)C(N(R₁))(N(R₁)(R_{6b})), —N(R₁)C(N(R_{6b}))(N(R₁)₂), —N(R_{6b})C(N(R₁))(N(R₁)₂), —N(R_{6b})C(N(R_{6b}))(N(R₁)₂), —N(R_{6b})C(N(R₁))(N(R₁)(R_{6b})), —N(R₁)C(N(R_{6b}))(N(R₁)(R_{6b})), —N(R₁)C(N(R₁))(N(R_{6b})₂), —N(R_{6b})C(N(R_{6b}))(N(R₁)(R_{6b})), —N(R_{6b})C(N(R₁))(N(R_{6b})₂), —N(R₁)C(N(R_{6b}))(N(R_{6b})₂), —N(R_{6b})C(N(R_{6b}))(N(R_{6b})₂), ═O, ═S, ═N(R₁) or ═N(R_{6b});

R₄ is independently alkyl of 1 to 12 carbon atoms, alkenyl of 2 to 12 carbon atoms, or alkynyl of 2 to 12 carbon atoms;

R₅ is independently R₄ wherein each R₄ is substituted with 0 to 3 R₃ groups;

R₅ₐ is independently alkylene of 1 to 12 carbon atoms, alkenylene of 2 to 12 carbon atoms, or alkynylene of 2-12 carbon atoms any one of which alkylene, alkenylene or alkynylene is substituted with 0-3 R₃ groups;

R₆ₐ is independently H or an ether- or ester-forming group;

R_{6b} is independently H, a protecting group for amino or the residue of a carboxyl-containing compound;

R_{6c} is independently H or the residue of an amino-containing compound;

W₁ is a group comprising an acidic hydrogen, a protected acidic group, or an R_{6c} amide of the group comprising an acidic hydrogen;

W₂ is a group comprising a basic heteroatom or a protected basic heteroatom, or an R_{6b} amide of the basic heteroatom;

W₃ is W₄ or W₅;

W₄ is R₅ or —C(O)R₅, —C(O)W₅, —SO₂R₅, or —SO₂W₅;

W₅ is carbocycle or heterocycle wherein W₅ is independently substituted with 0 to 3 R₂ groups;

W₆ is —R₅, —W₅, —R₅ₐW₅, —C(O)OR₆ₐ, —C(O)R_{6c}, —C(O)N(R_{6b})₂, —C(NR_{6b})(N(R_{6b})₂), —C(NR_{6b})(N(H)(R_{6b})), —C(N(H)(N(R_{6b})₂), —C(S)N(R_{6b})₂, or —C(O)R₂;

X₁ is a bond, —O—, —N(H)—, —N(W₆)—, —S—, —SO—, or —SO₂—; and

each m₁ is independently an integer from 0 to 2; provided, however, that compounds are excluded wherein U₁ is H or —CH₂CH(OH)CH₂(OH);

and the salts, solvates, resolved enantiomers and purified diastereomers thereof.

In another embodiment of the invention a compound or composition of the invention is provided that further comprises a pharmaceutically-acceptable carrier.

In another embodiment of the invention the activity of neuraminidase is inhibited by a method comprising the step of treating a sample suspected of containing neuraminidase with a compound or composition of the invention.

Another embodiment of the invention provides a method for inhibiting the activity of neuraminidase comprising the step of contacting a sample suspected of containing neuraminidase with the composition embodiments of the invention.

Another embodiment of this invention is a method for the treatment or prophylaxis of viruses, particularly influenza virus infection in a host comprising administration to the host, by a route other than topically to the respiratory tract, of a therapeutically effective dose of an antivirally active compound described in WO 91/16320, WO 92/06691 or U.S. Pat. No. 5,360,817.

In other embodiments, novel methods for synthesis of the compounds of this invention are provided. In one such embodiment, a method is provided for using a compound of the formula 281 wherein the method comprises treating compound 281 with a compound of the formula R₅—X₁—H to form a compound of the formula 281.1

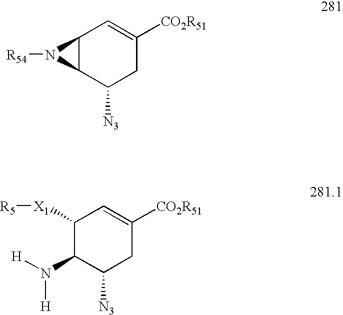

wherein:

X₁ and R₅ are as described above;

R₅₁ is an acid stable protecting group for a carboxylic acid; and

R₅₄ aziridine activating group.

In another embodiment, a method is provided for using a compound of the formula:

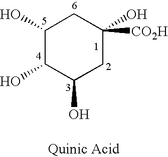

wherein the method comprises treating Quinic acid with a geminal dialkoxyalkane or geminal dialkoxy cycloalkane and acid to form a compound of the formula:

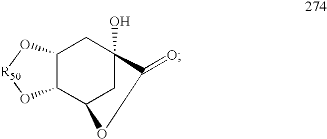

treating compound 274 with a metal alkoxide and an alkanol to form a compound of the formula:

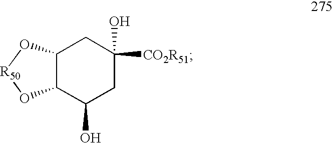

treating compound 275 with a sulfonic acid halide and an amine to form a compound of the formula:

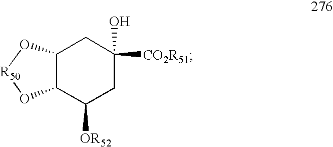

treating compound 276 with a dehydrating agent followed by an acid and an alkanol to form a compound of the formula:

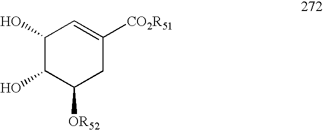

wherein:

R₅₀ is a 1,2 diol protecting group;

R₅₁ is an acid stable carboxylic acid protecting group; and

R₅₂ is a hydroxy activating group.

### BRIEF DESCRIPTION OF THE DRAWINGS

FIGS. 1 and 2 depict the arterial oxygen saturation (SaO₂) levels of influenza-A infected mice treated with varying i.p. doses of GG167 (4-guanidino-2,4-dideoxy-2,3-dehydro-N-acetylneuraminic acid), a known anti-influenza compound (FIG. 1) and compound 203 of this invention (FIG. 2): 50, 10, 2 and 0.5 mpk (mg/kg/day) of test compounds and saline control are designated, respectively, by squares, solid circles, triangles, diamonds and open circles. In all Figures, *P<0.05, **P<0.01 compared to the saline controls.

FIGS. 3-5 compare the SaO₂ levels achieved in influenza A infected mice treated with p.o. doses of ribavirin (triangles), compound 203 (squares) and GG167 (solid circles); saline controls are open circles: FIG. 3: 150 mpk of each of compound 203 and GG167, 100 mpk ribavirin; FIG. 4: 50 mpk of each of compound 203 and GG167, 32 mpk of ribavirin; FIG. 5: 10 mpk of each of compound 203 and GG167, 10 mpk of ribavirin.

FIGS. 6-8 depict the SaO₂ levels in influenza A infected mice treated with low p.o. doses of compounds 262 (circles) and 260 (solid squares) and GG167 (triangles); saline controls are open circles and uninfected controls are open squares: FIG. 6: mpk of each of the test compounds; FIG. 7: 1 mpk of each test compound; FIG. 8: 0.1 mpk of each test compound.

### DETAILED DESCRIPTION

### Compositions of the Invention

The compounds of this invention exclude compounds heretofore known. However, as will be further apparent below in other embodiments it is within the invention to use for antiviral purposes known compounds heretofore only produced and used as intermediates in the preparation of antiviral compounds. With respect to the United States, the compounds or compositions herein exclude compounds that are anticipated under 35 USC § 102 or obvious under 35 USC § 103. In particular, the claims herein shall be construed as excluding the compounds which are anticipated by or not possessing novelty over WO 91/16320, WO 92/06691, U.S. Pat. No. 5,360,817 or Chandler, M. et al., “J. Chem. Soc. Perkin Trans. 1”, 1189-1197 (1995).

The foregoing notwithstanding, in an embodiment of the invention one identifies compounds that may fall within the generic scope of WO 91/16320, WO 92/06691, or U.S. Pat. No. 5,360,817 but which have (a) formula Ia of the '320 application, (b) carbon for group “A” in the '320 application, and (c) R⁵ of the '320 and '691 applications being “—CH₂YR⁶, —CHYR⁶CH₂YR⁶ or —CHYR⁶CHYR⁶CH₂YR⁶” where YR⁶ cannot be either OH or protected OH in which the protecting group is capable of hydrolysis to yield the free OH under conditions of the human gastrointestinal tract, i.e. the compounds are stable to hydrolysis in the gastrointestinal tract. Thus, typically excluded from this embodiment are compounds of the '320 or '691 applications where R⁵ therein is acetyl or other carbacyl having 1-4 carbon atoms.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Such compounds are suitable for use in this embodiment. Note that simply because the compounds are stable to the gastrointestinal tract does not mean that they cannot be hydroyzed in vivo. Prodrugs typically will be stable in the digestive system but are substantially hydroyzed to the parental drug in the digestive lunem, liver or other metabolic organ, or within cells in general.

It should be understood, however, that other embodiments of this invention more fully described below contemplate the use of compounds that are in fact specifically disclosed in WO 91/16320, WO 92/06691, or U.S. Pat. No. 5,360,817, including those in which YR⁶ is free hydroxyl, or hydroxyl protected by a readily hydrolyzable group such as acetyl. In this instance, however, the compounds are delivered by novel routes of administration.

In another embodiment, the compounds herein exclude those in which

(a) E₁ is —CO₂H, —P(O)(OH)₂, —NO₂, —SO₂H, —SO₃H, tetrazolyl, —CH₂CHO, —CHO, or —CH(CHO)₂;

(b) G₁ is —CN, N₃, —NHR₂₀, NR₂₀, —OR₂₀, guanidino, SR₂₀, —N(R₂₀)ØO, —N(R₂₀)(OR₂₀), —N(H)(R₂₀)N(R₂₀)₂, unsubstituted pyrimidinyl, or unsubstituted (pyrimidinyl)methyl;

(c) T₁ is —NHR₂₀, —NO₂; and R₂₀ is H; an acyl group having 1 to 4 carbon atoms; a linear or cyclic alkyl group having 1 to 6 carbon atoms, or a halogen-substituted analogue thereof; an allyl group or an unsubstituted aryl group or an aryl substituted by a halogen, an OH group, an NO₂ group, an NH₂ group or a COOH group;

(d) each J₁ is H; and

(e) X₁ is a bond, —CH₂— or —CH₂CH₂—;

in which case W₆ is not H, W₇ or —CH₂W₇ wherein W₇ is H, —OR₆ₐ, —OR₁, —N(R₁)₂, —N(R₁)(R_{6b}), —N(R_{6b})₂, —SR₁, or —SR₆ₐ.

Also excluded herein are compounds described in WO 92/06691 at Page 9, Line 26, to Page 11, Line 5, which appear to include compounds of the formula II wherein:

(a) A₂ is O;

(b) E1 is COOH, P(O)(OH)₂, NO₂, SOOH, SO₃H, tetrazole, CH₂CHO, CHO, CH(CHO)₂ or where E₁ is COOH, P(O)(OH)₂, SOOH or SO₃H, an ethyl, methyl or pivaloyl ester thereof;

(c) G₁ is hydrogen, N(R^{20a})₂, SR^{20a} or OR^{20a};

(d) T₁ is —NHC(O)R^{20b}, where R_{20b} is an unsubstituted or halogen-substituted linear or cyclic alkyl group of 1 to 6 carbon atoms, or SR^{20a}, OR^{20a}, COOH or alkyl/aryl ester thereof, NO₂, C(R^{20a})₃, CH₂COOH or alkyl/aryl ester thereof, CH₂NO₂ or CH₂NHR^{20b};

(e) R^{20a} is hydrogen; an acyl group having 1 to 4 carbon atoms; a linear or cyclic alkyl group having 1 to 6 carbon atoms, or a halogen-substituted analogue thereof; or an unsubstituted aryl group or an aryl substituted by a halogen, an allyl group, an OH group, an NO₂ group, an NH₂ group or a COOH group;

(f) J₁ is H and J₁ₐ is H, OR^{20a}, F, Cl, Br, CN, NHR₂₀ₐ, SR^{20a} or CH₂X wherein X is NHR^{20a}, halogen or OR^{20a};

(g) J₂ is H or J₂ₐ is hydrogen, N(R^{20a})₂, SR^{20a} or OR^{20a};

(h) U₁ is CH₂YR^{20a}, CHYR²OCH₂YR^{20a} or CHYR^{20a}CHYR^{20a}CH₂YR^{20a} where Y is O, S or H, and successive Y moieties in U₁ are the same or different and R^{20a} represents a covalent bond when Y is hydrogen and

and pharmacologically acceptable salts or derivatives thereof.

In a further embodiment, the compounds of this invention are those in which U₁ is not —CH₂OH, —CH₂OAc, or —CH₂OCH₂Ph.

In a further embodiment, the compounds of this invention are those in which E₁ is not —CH₂OH, —CH₂OTMS, or —CHO.

In a further embodiment, the compounds of this invention are those in which U₁ is not bonded directly to the nuclear ring by a carbon atom or U₁ is not substituted with hydroxyl or hydroxyester, in particular U₁ is not polyhydroxyalkane, especially —CH(OH)CH(OH)CH₂OH. In a further embodiment, U₁ is a branched chain group R₅ as described below or a carbocycle which is substituted with at least one group R₅. In a further embodiments, excluded from the invention are compounds of the formula:

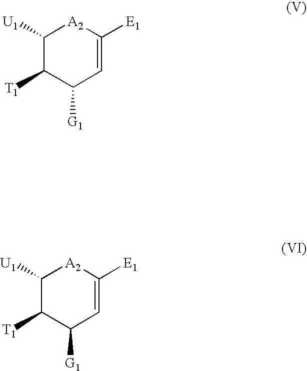

wherein:

1. In formula (V):

A₂ is —O— or —CH₂—;

E₁ is —CO₂H;

G₁ is —N(H)(C(NH)(NH₂));

T₁ is —N(H)(Ac); and

U₁ is of the formula:

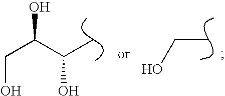

2. In formula (V):

A₂ is —O— or —CH₂—;

E₁ is —CO₂H;

G₁ is —NH₂;

T₁ is —N(H)(Ac); and

U₁ is —CH₂OH;

3. In formula (V):

A₂ —CH₂—;

E₁ is —CH₂OH or —CH₂OTMS;

G₁ is —N₃;

T₁ is —N(H)(Ac); and

U₁ is —CH₂OCH₂Ph;

4. In formula (V):

A₂ —CH₂—;

E₁ is —CO₂H or —CO₂CH₃;

G₁ is —N₃;

T₁ is —N(H)(Ac); and

U₁ is —CH₂OH;

5. In formula (V):

A₂ —CH₂—;

E₁ is —CO₂H, —CHO, or —CH₂OH;

G₁ is —N₃;

T₁ is —N(H)(Ac); and

U₁ is —CH₂OCH₂Ph;

6. In formula (VI):

A₂—CH₂—;

E₁ is —CO₂H;

G₁ is —OCH₃;

T₁ is —NH₂; and

U₁ is —CH₂OH; and

7. In formula (VI):

A₂ —CH₂—;

E₁ is —CO₂H;

G₁ is —OCH₃;

T₁ is —N(H)(Ac); and

U₁ is —CH₂OAc.

Whenever a compound described herein is substituted with more than one of the same designated group, e.g., “R₁” or “R₆ₐ”, then it will be understood that the groups may be the same or different, i.e., each group is independently selected.

“Heterocycle” as used herein includes by way of example and not limitation these heterocycles described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (ohn Wiley & Sons, New York, 1950 to present), in particular Volumes 13, 14, 16, 19, and 28; and “J. Am. Chem. Soc.”, 82:5566 (1960).

Examples of heterocycles include by way of example and not limitation pyridyl, thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathiinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or 0-carboline. Still more typically, nitrogen bonded heterocycles include 1-aziridyl, 1-azetedyl, 1-pyrrolyl, 1-imidazolyl, 1-pyrazolyl, and 1-piperidinyl.

“Alkyl” as used herein, unless stated to the contrary, is C₁-C₁₂ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (-CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃). Examples of alkyl groups appear in Table 2 as groups 2-5, 7, 9, and 100-399.

The compositions of the invention comprise compounds of either formula:

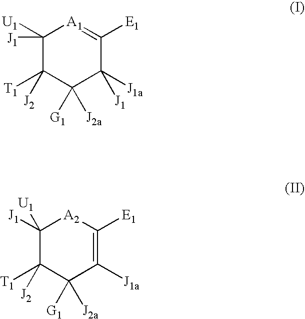

In the typical embodiment, the compounds of Formula I are chosen.

J₁ and J₁ₐ are independently R₁, Br, Cl, F, I, CN, NO₂ or N₃, typically R₁ or F, more typically H or F, more typically yet H.

J₂ and J₂ₐ are independently H or R₁, typically H.

A₁ is —C(J₁)═, or —N═, typically —C(J₁)═, more typically —CH═.

A₂ is —C(J₁)₂—, —N(J₁)—, —N(O)(J₁)—, —N(O)═, —S—, —S(O)—, —S(O)₂— or —O—, typically —C(J₁)₂—, —N(J₁)—, —S—, or —O—, more typically —C(J₁)₂—, or —O—, more typically yet —CH₂— or —O—, still more typically —CH₂—.

E₁ is —(CR₁R₁)ₘ₁W₁.

Typically, R₁ is H or alkyl of 1 to 12 carbon atoms, usually H or an alkyl of 1 to 4 or 5 to 10 carbon atoms, still more typically, H or an alkyl of 1, 2, 3, 4, 5, 6, 7, 8, 9, 10, 11, or 12 carbon atoms, more typically yet, H or an alkyl of 1 to 3 carbon atoms selected from methyl, ethyl, n-propyl, and i-propyl. Most typically R₁ is H.

m1 is an integer of 0 to 2, typically 0 or 1, most typically 0.

m2 is an integer of 0 to 1.

m3 is an integer of 1 to 3.

W₁ is a group comprising an acidic hydrogen, a protected acidic group or an R_{6c} amide of the group comprising an acidic hydrogen which, within the context of the invention, means a group having a hydrogen atom that can be removed by a base yielding an anion or its corresponding salt or solvate. The general principles of acidity and basicity of organic materials are well understood and are to be understood as defining W₁. They will not be detailed here. However, a description appears in Streitwieser, A.; and Heathcock, C. H.; “Introduction to Organic Chemistry, Second Edition” (Macmillan, New York, 1981), pages 60-64. Generally, acidic groups of the invention have pK values less than that of water, usually less than pK=10, typically less than pK=8, and frequently less than pK=6. They include tetrazoles and the acids of carbon, sulfur, phosphorous and nitrogen, typically the carboxylic, sulfuric, sulfonic, sulfinic, phosphoric and phosphonic acids, together with the R_{6c} amides and R_{6b} esters of those acids (R_{6c} and R_{6b} are defined below). Exemplary W₁ are —CO₂H, —CO₂R₆ₐ. —OSO₃H, —SO₃H, —SO₂H, —OPO₃H₂, —PO₃(R₆ₐ)₂, —PO₃H₂, —PO₃(H)(R₆ₐ), and —OPO₃(R₆ₐ)₂. E₁ typically is W₁, and W₁ typically is —CO₂H, —CO₂R₆ₐ, —CO₂R₄ or CO₂R₁, and most typically is CO₂R₁₄ wherein R₁₄ is normal or terminally secondary C₁-C₆ alkyl.

W₁ may also be a protected acidic group, which, within the context of the invention means an acidic group as described above that has been protected by one of the groups commonly used in the art for such groups and are described below under R₆ₐ. More typically, protected W₁ is —CO₂R₁, —SO₃R₁, —S(O)OR₁, —P(O)(OR₁)₂, —C(O)NHSO₂R₄, or —SO₂NHC(O)—R₄, wherein R₁ and R₄ are defined above.

Most typically, E₁ is selected from —C(O)O(CH₂)_{b}CH((CH₂)_{c}CH₃)₂ where b=0 to 4, c=0 to 4, and b+c=1 to 4, or from the group of

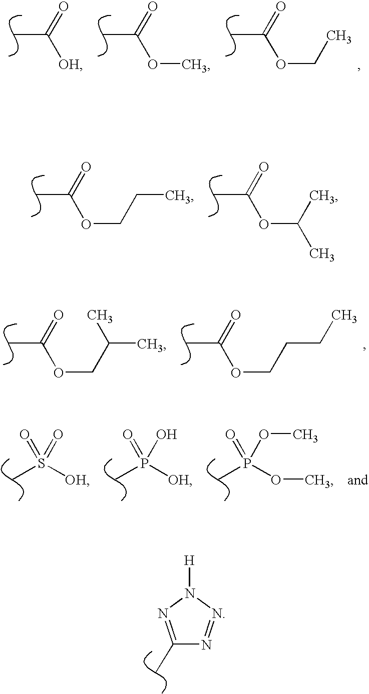

Exemplary E₁ groups are listed in Tables 3a through 3b.

G₁ is N₃, —CN, —OH, OR₆ₐ, —NO₂ or —(CR₁R₁)ₘ₁W₂, wherein R₁ and m1 are defined above. Ordinarily, G₁ is —(CR₁R₁)ₘ₁W₂.

W₂ is a group comprising a basic heteroatom, a protected basic heteroatom or an R_{6b} amide of the basic heteroatom. W₂ generally comprises a basic heteroatom, which, within the context of the invention means an atom other than carbon which is capable of protonation, typically by an acidic hydrogen having an acidity in the range described above for W₁. The basic principles of basicity are described in Streitwieser and Heathcock (op. cit.) and provide meaning for the term basic heteroatom as will be understood by those ordinarily skilled in the art. Generally, the basic heteroatoms employed in the compounds of the invention have pK values for the corresponding protonated form that are in the range of values described above for W₁. Basic heteroatoms include the heteroatoms common in organic compounds which have an un-shared, non-bonding, n-type, or the like, electron pair. By way of example and not limitation, typical basic heteroatoms include the oxygen, nitrogen, and sulfur atoms of groups such as alcohols, amines, amidines, guanidines, sulfides, and the like, frequently, amines, amidines and guanidines. Ordinarily, W₂ is amino or an amino alkyl (generally lower alkyl C₁ to C₆) group such as aminomethyl, aminoethyl or aminopropyl; an amidinyl, or an amidinoalkyl group such as amidinomethyl, amidinoethyl, or amidinopropyl; or guanidinyl, or a guanidinoalkyl group such as guanidinomethyl, guanidinoethyl, or guanidinopropyl (in each instance wherein the alkyl group serves to bridge the basic substituent to the carbocyclic ring). More typically, W₂ is amino, amidino, guanidino, heterocycle, heterocycle substituted with 1 or 2 amino or guanidino groups (usually 1), or an alkyl of 2 to 3 carbon atoms substituted with amino or guanidino, or such alkyl substituted with an amino and a second group selected from the group consisting of hydroxy and amino. The heterocycles useful as W₂ include typically N or S-containing 5 or 6 membered rings, wherein the ring contains 1 or 2 heteroatoms. Such heterocycles generally are substituted at ring carbon atoms. They may be saturated or unsaturated and may be linked to the core cyclohexene by lower alkyl (m1=1 or 2) or by —NR₁—. Still more typically, W₂ is —NHR₁, —C(NH)(NH₂), —NR₁—C(NR₁) (NR₁R₃), —NH—C(NH)(NHR₃), —NH—C(NH)(NHR₁), —NH—C(NH)NH₂, —CH(CH₂NHR₁)(CH₂OH), —CH(CH₂NHR₁)(CH₂NHR₁), —CH(NHR₁), —(CR₁R₁)ₘ₂—CH(NHR₁)R₁, —CH(O H)—(CR₁R₁)ₘ₂—CH(NHR₁)R₁, or —CH(NHR₁)—(CR₁R₁)ₘ₂—CH(OH)R₁, —(CR₁R₁)ₘ₂—S—C(NH)NH₂, —N═C(NHR₁)(R₃), —N═C(SR₁)N(R₁)₂, —N(R₁)C(NH)N(R₁)C═N, or —N═C(NHR₁)(R₁); wherein each m2 is ordinarly O, and ordinarily R₁ is H and R₃ is C(O)N(R₁)₂.

W₂ optionally is a protected basic heteroatom which within the context of the invention means a basic heteroatom as described above that has been protected by R_{6b} such as one of the groups common in the art. Such groups are described in detail in Greene (op. cit.) as set forth below. Such groups include by way of example and not limitation, amides, carbamates, amino acetals, imines, enamines, N-alkyl or N-aryl phosphinyls, N-alkyl or N-aryl sulfenyls or sulfonyls, N-alkyl or N-aryl silyls, thioethers, thioesters, disulfides, sulfenyls, and the like. In some embodiments, the protecting group R_{6b} will be cleavable under physiological conditions, typically it will be cleavable in vivo where, for example, the basic heteroatom forms an amide with an organic acid or an amino acid such as a naturally occurring amino acid or a polypeptide as described below for the R₆ₐ group.

Typically G₁ is selected from the group consisting of:

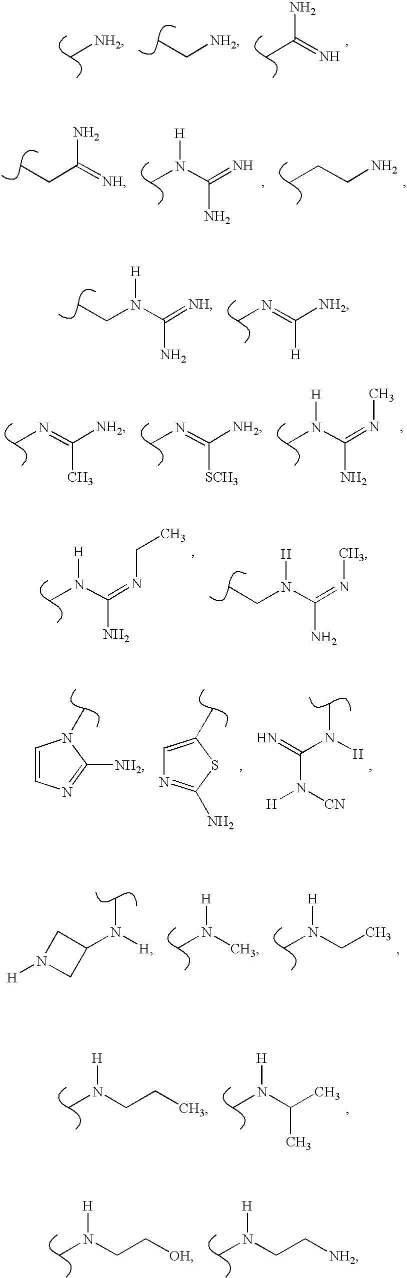

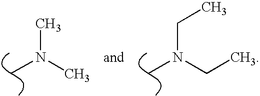

Further exemplary G₁ groups are listed in Table 4.

T₁ is —NR₁W₃, —R₃, —R₅ or heterocycle, or is taken together with U₁ or G₁ to form a group having the structure

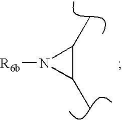

where R_{6b} is defined below, and R₁ and W₃ are defined above. Typically T₁ is —NR₁, W₃ or heterocycle. Generally T₁ is selected from the group consisting of:

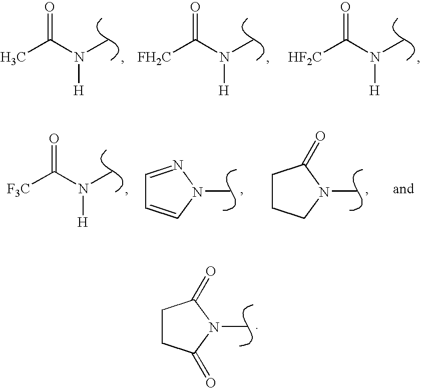

Exemplary T₁ groups are listed in Table 5.

W₃ is W₄ or W₅, wherein W₄ is R₅ or —C(O)R₅, —C(O)W₅, —SO₂R₅, or —SO₂W₅. Typically, W₃ is —C(O)R₅ or W₅.

R₂ is independently R₃ or R₄ as defined below, with the proviso that each R₄ is independently substituted with 0 to 3 R₃ groups;

R₃ is independently F, Cl, Br, I, —CN, N₃, —NO₂, OR₆ₐ, —OR₁, —N(R₁)₂, —N(R₁)(R_{6b}), —N(R_{6b})₂, —SR₁, —SR₆ₐ, —S(O)R₁, —S(O)₂R₁, —S(O)OR₁, —S(O)OR₆ₐ, —S(O)₂OR₁, —S(O)₂OR₆ₐ, —C(O)OR₁, —C(O)R_{6c}, —C(O)OR₆ₐ, —OC(O)R₁, —N(R₁)(C(O)R₁), —N(R_{6b})(C(O)R₁), —N(R₁)(C(O)OR₁), —N(R_{6b})(C(O)OR₁), —C(O)N(R₁)₂, —C(O)N(R_{6b})(R₁), —C(O)N(R_{6b})₂, —C(NR₁)(N(R₁)₂), —C(N(R_{6b}))(N(R₁)₂), —C(N(R₁))(N(R₁)(R_{6b})), —C(N(R_{6b}))(N(R₁)(R_{6b})), —C(N(R₁))(N(R_{6b})₂), —C(N(R_{6b}))(N(R_{6b})₂), —N(R₁)C(N(R₁))(N(R₁)₂), —N(R₁)C(N(R₁))(N(R₁)(R_{6b})), —N(R₁)C(N(R_{6b}))(N(R₁)₂), —N(R_{6b})C(N(R₁))(N(R₁)₂), —N(R_{6b})C(N(R_{6b}))(N(R₁)₂), —N(R_{6b})C(N(R₁))(N(R₁)(R_{6b})), —N(R₁)C(N(R_{6b}))(N(R₁)(R_{6b})), —N(R₁)C(N(R₁))(N(R_{6b})₂), —N(R_{6b})C(N(R_{6b}))(N(R₁)(R_{6b})), —N(R_{6b})C(N(R₁))(N(R_{6b})₂), —N(R₁)C(N(R_{6b}))(N(R_{6b})₂), —N(R_{6b})C(N(R_{6b}))(N(R_{6b})₂), ═O, ═S, ═N(R₁), ═N(R_{6b}) or W₅. Typically R₃ is F, Cl, —CN, N₃, NO₂, —OR₆ₐ, —OR₁, —N(R₁)₂, —N(R₁)(R_{6b}), —N(R_{6b})₂, —SR₁, —SR₆ₐ, —C(O)OR₁, —C(O)R_{6c}, —C(O)OR₆ₐ, —OC(O)R₁, —NR₁C(O)R₁, —N(R_{6b})C(O)R₁, —C(O)N(R₁)₂, —C(O)N(R_{6b})(R₁), —C(O)N(R_{6b})₂, or ═O. More typical R₃ groups comprising R_{6b} include —C(O)N(R_{6b})₂ or —C(O)N(R_{6b})(R₁). More typically yet R₃ is F, Cl, —CN, N₃, —OR₁, —N(R₁)₂, —SR₁, —C(O)OR₁, —OC(O)R₁, or ═O. More typically still, R₃ is F, —OR₁, —N(R₁)₂, or ═O. In the context of the present application, “═O” denotes a double bonded oxygen atom (oxo), and “═S” ═N(R_{6b}) and “═N(R₁)” denote the sulfur and nitrogen analogs.

R₄ is alkyl of 1 to 12 carbon atoms, and alkynyl or alkenyl of 2 to 12 carbon atoms. The alkyl R₄'s are typically of 1, 2, 3, 4, 5, 6, 7, 8, 9, 10, 11, or 12 carbon atoms and the alkenyl and alkynyl R₄'s are typically of 2, 3, 4, 5, 6, 7, 8, 9, 10, 11, or 12 carbon atoms. R₄ ordinarily is alkyl (as defined above). When R₄ is alkenyl it is typically ethenyl(—CH═CH₂), 1-prop-1-enyl(—CH═CHCH₃), 1-prop-2-enyl(—CH₂CH═CH₂), 2-prop-1-enyl(—C(=CH₂)(CH₃)), 1-but-1-enyl (—CH═CHCH₂CH₃), 1-but-2-enyl(—CH₂CH═CHCH₃), 1-but-3-enyl (—CH₂CH₂CH═CH₂), 2-methyl-1-prop-1-enyl(—CH═C(CH₃)₂), 2-methyl-1-prop-2-enyl(—CH₂C(═CH₂)(CH₃)), 2-but-1-enyl(—C(═CH₂)CH₂CH₃), 2-but-2-enyl(—C(CH₃)═CHCH₃), 2-but-3-enyl(—CH(CH₃)CH═CH₂), 1-pent-1-enyl(—C═CHCH₂CH₂CH₃), 1-pent-2-enyl(—CHCH═CHCH₂CH₃), 1-pent-3-enyl(—CHCH₂CH═CHCH₃), 1-pent-4-enyl(—CHCH₂CH₂CH═CH₂), 2-pent-1-enyl(—C(═CH₂)CH₂CH₂CH₃), 2-pent-2-enyl(—C(CH₃)═CH₂CH₂CH₃, 2-pent-3-enyl(—CH(CH₃)CH═CHCH₃), 2-pent-4-enyl(—CH(CH₃)CH₂CH═CH₂) or 3-methyl-1-but-2-enyl(—CH₂CH═C(CH₃)₂). More typically, R₄ alkenyl groups are of 2, 3 or 4 carbon atoms. When R₄ is alkynyl it is typically ethynyl (—C+CH), 1-prop-1-ynyl(—C+CCH₃), 1-prop-2-ynyl(—CH₂C+CH), 1-but-1-ynyl(—C+CCH₂CH₃), 1-but-2-ynyl(—CH₂C+CCH₃), 1-but-3-ynyl (—CH₂CH₂C+CH), 2-but-3-ynyl(CH(CH₃)C+CH), 1-pent-1-ynyl (—C+CCH₂CH₂CH₃), 1-pent-2-ynyl(—CH₂C+CCH₂CH₃), 1-pent-3-ynyl (—CH₂CH₂C+CCH₃) or 1-pent-4-ynyl(—CH₂CH₂CH₂C+CH). More typically, R₄ alkynyl groups are of 2, 3 or 4 carbon atoms.

R₅ is R₄, as defined above, or R₄ substituted with 0 to 3 R₃ groups. Typically R₅ is an alkyl of 1 to 4 carbon atoms substituted with 0 to 3 fluorine atoms.

R₅ₐ is independently alkylene of 1 to 12 carbon atoms, alkenylene of 2 to 12 carbon atoms, or alkynylene of 2-12 carbon atoms any one of which alkylene, alkenylene or alkynylene is substituted with 0-3 R₃ groups. As defined above for R₄, R₅ₐ's are of 1, 2, 3, 4, 5, 6, 7, 8, 9, 10, 11, or 12 carbon atoms when alkylene and of 2, 3, 4, 5, 6, 7, 8, 9, 10, 11, or 12 carbon atoms when alkenylene or alkynylene. Each of the typical R₄ groups is a typical R₅ₐ group with the proviso that one of the hydrogen atoms of the described R₄ group is removed to form the open valence to a carbon atom through which the second bond to the R₅ₐ is attached.

R₁₄ is normal or terminally secondary C₁-C₆ alkyl.

W₅ is a carbocycle or heterocycle, with the proviso that each W₅ is independently substituted with 0 to 3 R₂ groups. W₅ carbocycles and T₁ and W₅ heterocycles are stable chemical structures. Such structures are isolatable in measurable yield, with measurable purity, from reaction mixtures at temperatures from −78° C. to 200° C. Each W₅ is independently substituted with 0 to 3 R₂ groups. Typically, T₁ and W₅ are a saturated, unsaturated or aromatic ring comprising a mono- or bicyclic carbocycle or heterocycle. More typically, T₁ or W₅ has 3 to 10 ring atoms, still more typically, 3 to 7 ring atoms, and ordinarily 3 to 6 ring atoms. The T₁ and W₅ rings are saturated when containing 3 ring atoms, saturated or monounsaturated when containing 4 ring atoms, saturated, or mono- or diunsaturated when containing 5 ring atoms, and saturated, mono- or diunsaturated, or aromatic when containing 6 ring atoms. Unsaturation of the W₅ rings include internal and external unsaturation wherein the external incorporates a ring atom.

When W₅ is carbocyclic, it is typically a 3 to 7 carbon monocycle or a 7 to 12 carbon atom bicycle. More typically, W₅ monocyclic carbocycles have 3 to 6 ring atoms, still more typically 5 or 6 ring atoms. W₅ bicyclic carbocycles typically have 7 to 12 ring atoms arranged as a bicyclo [4,5], [5,5], [5,6] or [6,6] system, still more typically, 9 or 10 ring atoms arranged as a bicyclo [5,6] or [6,6] system. Examples include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl.

A T₁ or W₅ heterocycle is typically a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S). More typically, T₁ and W₅ heterocyclic monocycles have 3 to 6 ring atoms (2 to 5 carbon atoms and 1 to 2 heteroatoms selected from N, O, and S), still more typically, 5 or 6 ring atoms (3 to 5 carbon atoms and 1 to 2 heteroatoms selected from N and S). T₁ and W₅ heterocyclic bicycles have 7 to 10 ring atoms (6 to 9 carbon atoms and 1 to 2 heteroatoms selected from N, O, and S) arranged as a bicyclo [4,5], [5,5], [5,6], or [6,6] system, still more typically, 9 to 10 ring atoms (8 to 9 carbon atoms and 1 to 2 hetero atoms selected from N and S) arranged as a bicyclo [5,6] or [6,6] system.

Typically T₁ and W₅ heterocycles are selected from pyridyl, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, or pyrrolyl.

More typically, the heterocycle of T₁ and W₅ is bonded through a carbon atom or nitrogen atom thereof. Still more typically T₁ heterocycles are bonded by a stable covalent bond through a nitrogen atom thereof to the cyclohexene ring of the compositions of the invention and W₅ heterocycles are bonded by a stable covalent bond through a carbon or nitrogen atom thereof to the cyclohexene ring of the compositions of the invention. Stable covalent bonds are chemically stable structures as described above.

W₅ optionally is selected from the group consisting of:

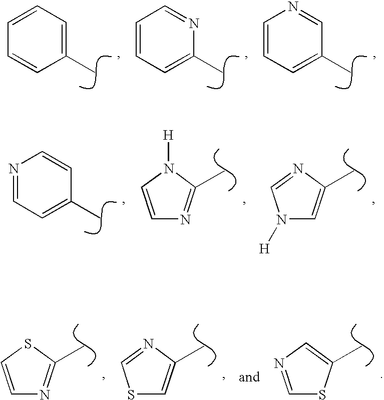

U₁ is H or —X₁W₆, but typically the latter.

X₁ is a bond, —O—, —N(H)—, —N(W₆)—, —N(OH)—, —N(OW₆)—, —N(NH₂)—, —N(N(H)(W₆))—, —N(N(W₆)₂)—, —N(H)N(W₆)—, —S—, —SO—, or —SO₂—; typically, X₁ is a bond, —O—, —N(H)—, —N(R₅)—, —N(OH)—, —N(OR₅)—, —N(NH₂)—, —N(N(H)(R₅))—, —N(N(R₅)₂)—, —N(H)N(R₅)—, —S—, —SO—, or —SO₂—, more typically X₁ is a bond, —O—, —NR₁—, —N(OR₁)—, —N(NR₁R₁)—, —S—, —SO—, or —SO₂—. Ordinarily X₁ is —O—, —NH—, —S—, —SO—, or —SO₂—.;

W₆ is —R₅, —W₅, —R₅ₐW₅, —C(O)OR₆ₐ, —C(O)R_{6c}, —C(O)N(R_{6b})₂, —C(NR_{6b})(N(R_{6b})₂), —C(NR_{6b})(N(H)(R_{6b})), —C(N(H)(N(R_{6b})₂), —C(S)N(R_{6b})₂, or —C(O)R₂, typically W₆ is —R₅, —W₅, or —R₅ₐW₅; in some embodiments, W₆ is R₁, —C(O)—R₁, —CHR₁W₇, —CH(R₁)ₐW₇, —CH(W₇)₂, (where, W₇ is monovalent a is 0 or 1, but is 0 when W₇ is divalent) or —C(O)W₇. In some embodiments, W₆ is —CHR₁W₇ or —C(O)W₇, or W₆ is —(CH₂)ₘ₁CH((CH₂)ₘ₃R₃)₂, —(CH₂)ₘ₁C((CH₂)ₘ₃R₃)₃; —(CH₂)ₘ₁CH((CH₂)ₘ₃R₅ₐW₅)₂; —(CH₂)ₘ₁CH((CH₂)ₘ₃R₃)((CH₂)ₘ₃R₅ₐW₅); —(CH₂)ₘ₁C((CH₂)ₘ₃R₃)₂(CH₂)ₘ₃R₅ₐW₅), (CH₂)ₘ₁C((CH₂)ₘ₃R₅ₐW₅)₃ or —(CH₂)ₘ₁C((CH₂)ₘ₃R₃)((CH₂)ₘ₃R₅ₐW₅)₂; and wherein m₃ is an integer from 1 to 3.

W₇ is R₃ or R₅, but typically is alkyl of 1 to 12 carbons substituted with 0 to 3 R₃ groups, the latter typically selected from the group consisting of —NR₁(R_{6b}), —N(R_{6b})₂, —OR₆ₐ, or SR₆ₐ. More typically, W₇ is —OR₁ or an alkyl of 3 to 12 carbon atoms substituted with OR₁.

In general, U₁ is R₁O—, —OCHR₁W₇,

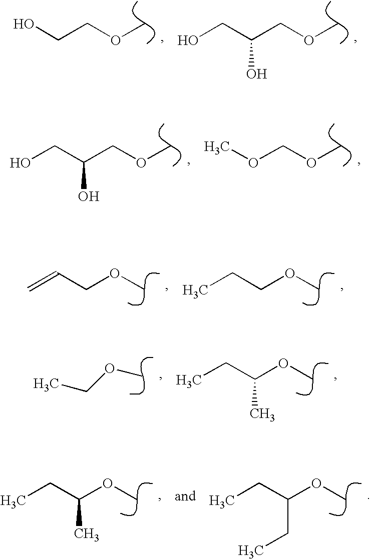

Exemplary U₁ groups are listed in Table 2.

An embodiment of the invention comprises a compound of the formula:

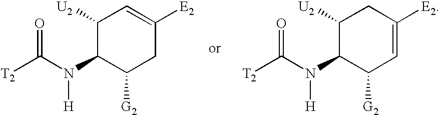

wherein E₂ is E₁, but is typically selected from the group consisting of:

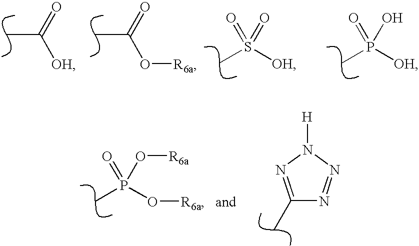

and wherein G₂ is G₁, but is typically selected from the group consisting of:

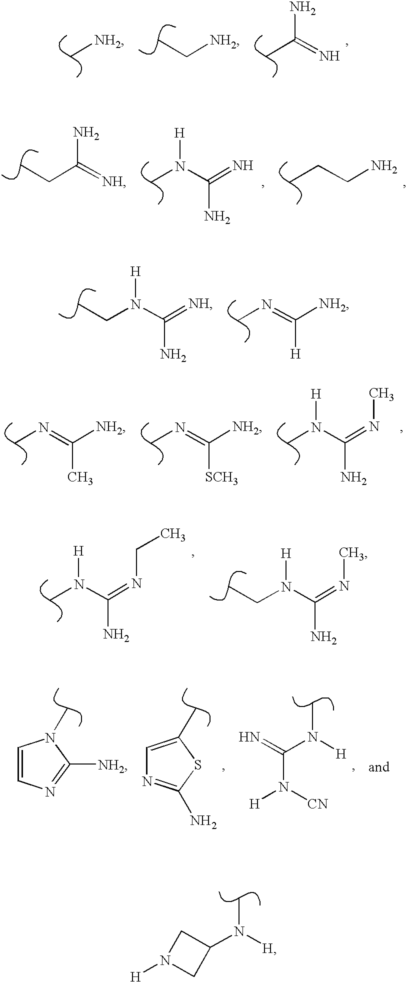

and wherein T₂ is R₄ or R₅. Generally, T₂ is alkyl of 1 to 2 carbon atoms substituted with 0 to 3 fluorine atoms.

U₂ is one of:

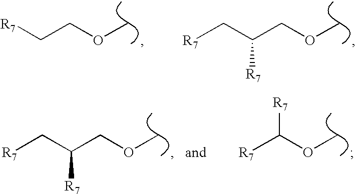

wherein R₇ is H, —CH₃, —CH₂CH₃, —CH₂CH₂CH₃, —OCH₃, —OAc (—O—C(O)CH₃), —OH, —NH₂, or —SH, typically H, —CH₃ or —CH₂CH₃.

Groups R₆ₐ and R_{6b} are not critical functionalities and may vary widely. When not H, their function is to serve as intermediates for the parental drug substance. This does not mean that they are biologically inactive. On the contrary, a principal function of these groups is to convert the parental drug into a prodrug, whereby the parental drug is released upon conversion of the prodrug in vivo. Because active prodrugs are absorbed more effectively than the parental drug they in fact often possess greater potency in vivo than the parental drug. When not hydrogen, R₆ₐ and R_{6b} are removed either in vitro, in the instance of chemical intermediates, or in vivo, in the case of prodrugs. With chemical intermediates, it is not particularly important that the resulting pro-functionality products, e.g. alcohols, be physiologically acceptable, although in general it is more desirable if the products are pharmacologically innocuous.

R₆ₐ is H or an ether- or ester-forming group. “Ether-forming group” means a group which is capable of forming a stable, covalent bond between the parental molecule and a group having the formula:

S—O—Vₐ(V₁)₃, S—O—Vₐ(V₁)(V₂), S—O—Vₐ(V₃)

S—O—V_{b}(V₁)₂, S—O—V_{b}(V₂), or S—O—V_{c}(V₁)

Wherein Vₐ is a tetravalent atom typically selected from C and Si; V_{b} is a trivalent atom typically selected from B, Al, N, and P, more typically N and P; V_{c} is a divalent atom typically selected from O, S, and Se, more typically S; V₁ is a group bonded to Vₐ, V_{b} or V_{c} by a stable, single covalent bond, typically V₁ is W₆ groups, more typically V₁ is H, R₂, W₅, or —R₅ₐW₅, still more typically H or R₂; V₂ is a group bonded to Vₐ or V_{b} by a stable, double covalent bond, provided that V₂ is not ═O, ═S or ═N—, typically V₂ is =C(V₁)₂ wherein V₁ is as described above; and V₃ is a group bonded to Vₐ by a stable, triple covalent bond, typically V₃ is +C(V₁) wherein V₁ is as described above.

“Ester-forming group” means a group which is capable of forming a stable, covalent bond between the parental molecule and a group having the formula:

S—O—Vₐ(V₁)(V₄), S—O—V_{b}(V₄), S—O—V_{d}(V₁)₂(V₄),

S—O—V_{d}(V₄)₂, S—O—Vₑ(V₁)₃(V₄), or S—O—Vₑ(V₁)(V₄)₂

Wherein Vₐ, V_{b}, and V₁, are as described above; V_{d} is a pentavalent atom typically selected from P and N; Vₑ is a hexavalent atom typically S; and V₄ is a group bonded to Vₐ, V_{b}, V_{d} or Vₑ by a stable, double covalent bond, provided that at least one V₄ is ═O, ═S or ═N—V₁, typically V₄, when other than ═O, ═S or ═N—, is ═C(V₁)₂ wherein V₁ is as described above.

Protecting groups for —OH functions (whether hydroxy, acid or other functions) are embodiments of “ether- or ester-forming groups”. Particularly of interest are ether- or ester-forming groups that are capable of functioning as protecting groups in the synthetic schemes set forth herein. However, some hydroxyl and thio protecting groups are neither ether- nor ester-forming groups, as will be understood by those skilled in the art, and are included with amides, discussed under R_{6c} below. R_{6c} is capable of protecting hydroxyl or thio groups such that hydrolysis from the parental molecule yields hydroxyl or thio.

In its ester-forming role, R₆ₐ typically is bound to any acidic group such as, by way of example and not limitation, a —CO₂H or —C(S)OH group, thereby resulting in —CO₂R₆ₐ. R₆ₐ for example is deduced from the enumerated ester groups of WO 95/07920.

Examples of R₆ₐ include

C₃-C₁₂ heterocyle (described above) or C₆-C₁₂ aryl. These aromatic groups optionally are polycyclic or monocyclic. Examples include phenyl, spiryl, 2- and 3-pyrrolyl, 2- and 3-thienyl, 2- and 4-imidazolyl, 2-, 4- and 5-oxazolyl, 3- and 4-isoxazolyl, 2-, 4- and 5-thiazolyl, 3-, 4- and 5-isothiazolyl, 3- and 4-pyrazolyl, 1-, 2-, 3- and 4-pyridinyl, and 1-, 2-, 4- and 5-pyrimidinyl,

C₃-C₁₂ heterocycle or C₆-C₁₂ aryl substituted with halo, R₁, R₁—O—C₁-C₁₂ alkylene, C₁-C₁₂ alkoxy, CN, NO₂, OH, carboxy, carboxyester, thiol, thioester, C₁-C₁₂ haloalkyl (1-6 halogen atoms), C₂-C₁₂ alkenyl or C₂-C₁₂ alkynyl. Such groups include 2-, 3- and 4-alkoxyphenyl (C₁-C₁₂ alkyl), 2-, 3-and 4-methoxyphenyl, 2-, 3- and 4-ethoxyphenyl, 2,3-, 2,4-, 2,5-, 2,6-, 3,4- and 3,5-diethoxyphenyl, 2- and 3-carboethoxy-4-hydroxyphenyl, 2- and 3-ethoxy-4-hydroxyphenyl, 2- and 3-ethoxy-5-hydroxyphenyl, 2- and 3-ethoxy-6-hydroxyphenyl, 2-, 3- and 4-O-acetylphenyl, 2-, 3- and 4-dimethylaminophenyl, 2-, 3- and 4-methylmercaptophenyl, 2-, 3- and 4-halophenyl (including 2-, 3- and 4-fluorophenyl and 2-, 3- and 4-chlorophenyl), 2,3-, 2,4-, 2,5-, 2,6-, 3,4- and 3,5-dimethylphenyl, 2,3-, 2,4-, 2,5-, 2,6-, 3,4- and 3,5-biscarboxyethylphenyl, 2,3-, 2,4-, 2,5-, 2,6-, 3,4- and 3,5-dimethoxyphenyl, 2,3-, 2,4-, 2,5-, 2,6-, 3,4- and 3,5-dihalophenyl (including 2,4-difluorophenyl and 3,5-difluorophenyl), 2-, 3- and 4-haloalkylphenyl (1 to 5 halogen atoms, C₁-C₁₂ alkyl including 4-trifluoromethylphenyl), 2-, 3- and 4-cyanophenyl, 2-, 3- and 4-nitrophenyl, 2-, 3- and 4-haloalkylbenzyl (1 to 5 halogen atoms, C₁-C₁₂ alkyl including 4-trifluoromethylbenzyl and 2-, 3- and 4-trichloromethylphenyl and 2-, 3- and 4-trichloromethylphenyl), 4-N-methylpiperidinyl, 3-N-methylpiperidinyl, 1-ethylpiperazinyl, benzyl, alkylsalicylphenyl (C₁-C₄ alkyl, including 2-, 3- and 4-ethylsalicylphenyl), 2-,3- and 4-acetylphenyl, 1,8-dihydroxynaphthyl (—C₁₀H₆—OH) and aryloxy ethyl [C₆-C₉aryl (including phenoxy ethyl)], 2,2′-dihydroxybiphenyl, 2-, 3- and 4-N,N-dialkylaminophenol, —C₆H₄CH₂—N(CH₃)₂, trimethoxybenzyl, triethoxybenzyl, 2-alkyl pyridinyl (C₁₋₄ alkyl);

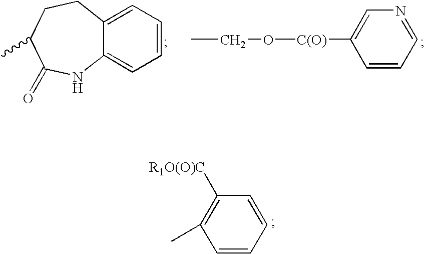

C₄-C₈ esters of 2-carboxyphenyl; and C₁-C₄ alkylene-C₃-C₆ aryl (including benzyl, —CH₂-pyrrolyl, —CH₂-thienyl, —CH₂-imidazolyl, —CH₂-oxazolyl, —CH₂-isoxazolyl, —CH₂-thiazolyl, —CH₂-isothiazolyl, —CH₂-pyrazolyl, —CH₂-pyridinyl and —CH₂-pyrimidinyl) substituted in the aryl moiety by 3 to 5 halogen atoms or 1 to 2 atoms or groups selected from halogen, C₁-C₁₂ alkoxy (including methoxy and ethoxy), cyano, nitro, OH, C₁-C₁₂ haloalkyl (1 to 6 halogen atoms; including —CH₂-CCl₃), C₁-C₁₂ alkyl (including methyl and ethyl), C₂-C₁₂ alkenyl or C₂-C₁₂ alkynyl;

alkoxy ethyl [C₁-C₆ alkyl including —CH₂—CH₂—O—CH₃ (methoxy ethyl)];

alkyl substituted by any of the groups set forth above for aryl, in particular OH or by 1 to 3 halo atoms (including —CH₃, —CH(CH₃)₂, —C(CH₃)₃, —CH₂CH₃, —(CH₂)₂CH₃, —(CH₂)₃CH₃, —(CH₂)₄CH₃, —(CH₂)₅CH₃, —CH₂CH₂F, —CH₂CH₂Cl, —CH₂CF₃, and —CH₂CCl₃);

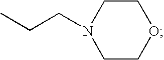

—N-2-propylmorpholino, 2,3-dihydro-6-hydroxyindene, sesamol, catechol monoester, —CH₂—C(O)—N(R¹)₂, —CH₂—S(O)(R¹), —CH₂—S(O)₂(R¹), —CH₂—CH(OC(O)CH₂R¹)—CH₂(OC(O)CH₂R¹), cholesteryl, enolpyruvate (HOOC—C(═CH₂)—), glycerol;

a 5 or 6 carbon monosaccharide, disaccharide or oligosaccharide (3 to 9 monosaccharide residues);

triglycerides such as α-D-β-diglycerides (wherein the fatty acids composing glyceride lipids generally are naturally occurring saturated or unsaturated C₆₋₂₆, C₆₋₁₈ or C₆₋₁₀ fatty acids such as linoleic, lauric, myristic, palmitic, stearic, oleic, palmitoleic, linolenic and the like fatty acids) linked to acyl of the parental compounds herein through a glyceryl oxygen of the triglyceride;

phospholipids linked to the carboxyl group through the phosphate of the phospholipid;

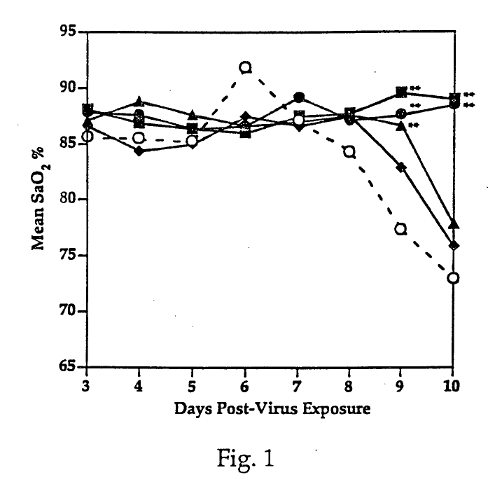

phthalidyl (shown in FIG. 1 of Clayton et al., “Antimicrob. Agents Chemo.” 5(6):670-671 [1974]);

cyclic carbonates such as (5-R_{d}-2-oxo-1,3-dioxolen-4-yl) methyl esters (Sakamoto et al., “Chem. Pharm. Bull.” 32(6)₂₂₄₁-2248 [1984]) where R_{d} is R₁, R₄ or aryl; and

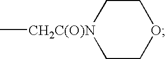

The hydroxyl groups of the compounds of this invention optionally are substituted with one of groups III, IV or V disclosed in WO94/21604, or with isopropyl.

As further embodiments, Table A lists examples of R₆ₐ ester moieties that for example can be bonded via oxygen to —C(O)O— and —P(O)(O—)₂ groups. Several R_{6c} amidates also are shown, which are bound directly to —C(O)— or —P(O)₂. Esters of structures 1-5, 8-10 and 16, 17, 19-22 are synthesized by reacting the compound herein having a free hydroxyl with the corresponding halide (chloride or acyl chloride and the like) and N ,N-dicyclohexyl-N-morpholine carboxamidine (or another base such as DBU, triethylamine, CsCO₃, N,N-dimethylaniline and the like) in DMF (or other solvent such as acetonitrile or N-methylpyrrolidone). When W₁ is phosphonate, the esters of structures 5-7, 11, 12, 21, and 23-26 are synthesized by reaction of the alcohol or alkoxide salt (or the corresponding amines in the case of compounds such as 13, 14 and 15) with the monochlorophosphonate or dichlorophosphonate (or another activated phosphonate).

TABLE A

| | |
|---|---|
| 1. | —CH₂—C(O)—N(R₁)₂ |
| 2. | —CH₂—S(O)(R₁) |
| 3. | —CH₂—S(O)₂(R₁) |
| 4. | —CH₂—O—C(O)—CH₂—C₆H₅ |
| 5. | 3-cholesteryl |
| 6. | 3-pyridyl |
| 7. | N-ethylmorpholino |
| 8. | —CH₂—O—C(O)—C₆H₅ |
| 9. | —CH₂—O—C(O)—CH₂CH₃ |
| 10. | —CH₂—O—C(O)—C(CH₃)₃ |
| 11. | —CH₂—CCl₃ |
| 12. | —C₆H₅ |
| 13. | —NH—CH₂—C(O)O—CH₂CH₃ |
| 14. | —N(CH₃)—CH₂—C(O)O—CH₂CH₃ |
| 15. | —NHR₁ |
| 16. | —CH₂—O—C(O)—C₁₀H₁₅ |
| 17. | —CH₂—O—C(O)—CH(CH₃)₂ |
| 18. | —CH₂—C#H(OC(O)CH₂R₁)—CH₂—(OC(O)CH₂R₁) |
| 19. | 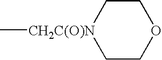 |
| 20. | 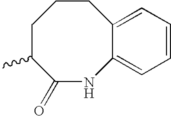 |
| 21. | 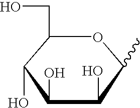 |
| 22. | 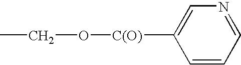 |
| 23. | 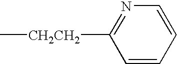 |
| 24. | 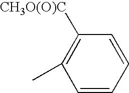 |
| 25. | 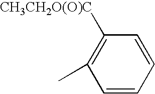 |
| 26. | 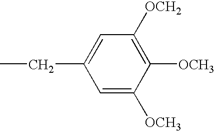 |

#—chiral center is (R), (S) or racemate.
Other esters that are suitable for use herein are described in EP 632,048.
R₆ₐ also includes “double ester” forming profunctionalities such as -

CH₂OC(O)OCH₃,

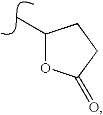

—CH₂SCOCH₃, —CH₂OCON(CH₃)₂, or alkyl- or aryl-acyloxyalkyl groups of the structure —CH(R₁ or W₅)O((CO)R₃₇) or —CH(R₁ or W₅)((CO)OR₃₈) (linked to oxygen of the acidic group) wherein R₃₇ and R₃₈ are alkyl, aryl, or alkylaryl groups (see U.S. Pat No. 4,968,788). Frequently R₃₇ and R₃₈ are bulky groups such as branched alkyl, ortho-substituted aryl, meta-substituted aryl, or combinations thereof, including normal, secondary, iso- and tertiary alkyls of 1-6 carbon atoms. An example is the pivaloyloxymethyl group. These are of particular use with prodrugs for oral administration. Examples of such useful R₆ₐ groups are alkylacyloxymethyl esters and their derivatives, including —CH(CH₂CH₂OCH₃)OC(O)C(CH₃)₃,

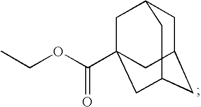

—CH₂OC(O)C₁₀H₁₅, —CH₂OC(O)C(CH₃)₃, —CH(CH₂OCH₃)OC(O)C(CH₃)₃, —CH(CH(CH₃)₂)OC(O)C(CH₃)₃, —CH₂OC(O)CH₂CH(CH₃)₂, —CH₂OC(O)C₆H₁₁, —CH₂OC(O)C₆H₅, —CH₂OC(O)C₁₀H₁₅, —CH₂OC(O)CH₂CH₃, —CH₂OC(O)CH(CH₃)₂, —CH₂OC(O)C(CH₃)₃ and —CH₂OC(O)CH₂C₆H₅.

For prodrug purposes, the ester typically chosen is one heretofore used for antibiotic drugs, in particular the cyclic carbonates, double esters, or the phthalidyl, aryl or alkyl esters.

As noted, R₆ₐ, R_{6c} and R_{6b} groups optionally are used to prevent side reactions with the protected group during synthetic procedures, so they function as protecting groups (PRT) during synthesis. For the most part the decision as to which groups to protect, when to do so, and the nature of the PRT will be dependent upon the chemistry of the reaction to be protected against (e.g., acidic, basic, oxidative, reductive or other conditions) and the intended direction of the synthesis. The PRT groups do not need to be, and generally are not, the same if the compound is substituted with multiple PRT. In general, PRT will be used to protect carboxyl, hydroxyl or amino groups. The order of deprotection to yield free groups is dependent upon the intended direction of the synthesis and the reaction conditions to be encountered, and may occur in any order as determined by the artisan.

A very large number of R₆ₐ hydroxy protecting groups and R_{6c} amide-forming groups and corresponding chemical cleavage reactions are described in “Protective Groups in Organic Chemistry”, Theodora W. Greene (John Wiley & Sons, Inc., New York, 1991, ISBN 0-471-62301-6) (“Greene”). See also Kocienski, Philip J.; “Protecting Groups” (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For R₆ₐ carboxylic acid, phosphonic acid, phosphonate, sulfonic acid and other protecting groups for W₁ acids see Greene as set forth below. Such groups include by way of example and not limitation, esters, amides, hydrazides, and the like.

In some embodiments the R₆ₐ protected acidic group is an ester of the acidic group and R₆ₐ is the residue of a hydroxyl-containing functionality. In other embodiments, an R_{6c} amino compound is used to protect the acid functionality. The residues of suitable hydroxyl or amino-containing functionalities are set forth above or are found in WO 95/07920. Of particular interest are the residues of amino acids, amino acid esters, polypeptides, or aryl alcohols. Typical amino acid, polypeptide and carboxyl-esterified amino acid residues are described on pages 11-18 and related text of WO 95/07920 as groups L1 or L2. WO 95/07920 expressly teaches the amidates of phosphonic acids, but it will be understood that such amidates are formed with any of the acid groups set forth herein and the amino acid residues set forth in WO 95/07920.

Typical R₆ₐ esters for protecting W₁ acidic functionalities are also described in WO 95/07920, again understanding that the same esters can be formed with the acidic groups herein as with the phosphonate of the '920 publication. Typical ester groups are defined at least on WO 95/07920 pages 89-93 (under R³¹ or R³⁵), the table on page 105, and pages 21-23 (as R). Of particular interest are esters of unsubstituted aryl such as phenyl or arylalkyl such benzyl, or hydroxy-, halo-, alkoxy-, carboxy- and/or alkylestercarboxy-substituted aryl or alkylaryl, especially phenyl, ortho-ethoxyphenyl, or C₁-C₄ alkylestercarboxyphenyl (salicylate C₁-C₁₂ alkylesters).

The protected acidic groups W₁, particularly when using the esters or amides of WO 95/07920, are useful as prodrugs for oral administration. However, it is not essential that the W₁ acidic group be protected in order for the compounds of this invention to be effectively administered by the oral route. When the compounds of the invention having protected groups, in particular amino acid amidates or substituted and unsubstituted aryl esters are administered systemically or orally they are capable of hydrolytic cleavage in vivo to yield the free acid.

One or more of the acidic hydroxyls are protected. If more than one acidic hydroxyl is protected then the same or a different protecting group is employed, e.g., the esters may be different or the same, or a mixed amidate and ester may be used.

Typical R₆ₐ hydroxy protecting groups described in Greene (pages 14-118) include Ethers (Methyl); Substituted Methyl Ethers (Methoxymethyl, Methylthiomethyl, t-Butylthiomethyl, (Phenyldimethylsilyl)methoxymethyl, Benzyloxymethyl, p-Methoxybenzyloxymethyl, (4-Methoxyphenoxy)methyl, Guaiacolmethyl, t-Butoxymethyl, 4-Pentenyloxymethyl, Siloxymethyl, 2-Methoxyethoxymethyl, 2,2,2-Trichloroethoxymethyl, Bis(2-chloroethoxy)methyl, 2-(Trimethylsilyl)ethoxymethyl, Tetrahydropyranyl, 3-Bromotetrahydropyranyl, Tetrahydropthiopyranyl, 1-Methoxycyclohexyl, 4-Methoxytetrahydropyranyl, 4-Methoxytetrahydrothiopyranyl, 4-Methoxytetrahydropthiopyranyl S,S-Dioxido, 1-[(2-Chloro-4-methyl)phenyl]-4-methoxypiperidin-4-yl, 35, 1,4-Dioxan-2-yl, Tetrahydrofuranyl, Tetrahydrothiofuranyl, 2,3,3a,4,5,6,7,7a-Octahydro-7,8,8-trimethyl-4,7-methanobenzofuran-2-yl)); Substituted Ethyl Ethers (1-Ethoxyethyl, 1-(2-Chloroethoxy)ethyl, 1-Methyl-1-methoxyethyl, 1-Methyl-1-benzyloxyethyl, 1-Methyl-1-benzyloxy-2-fluoroethyl, 2,2,2-Trichloroethyl, 2-Trimethylsilylethyl, 2-(Phenylselenyl)ethyl, t-Butyl, Allyl, p-Chlorophenyl, p-Methoxyphenyl, 2,4-Dinitrophenyl, Benzyl); Substituted Benzyl Ethers (p-Methoxybenzyl, 3,4-Dimethoxybenzyl, o-Nitrobenzyl, p-Nitrobenzyl, p-Halobenzyl, 2,6-Dichlorobenzyl, p-Cyanobenzyl, p-Phenylbenzyl, 2- and 4-Picolyl, 3-Methyl-2-picolyl N-Oxido, Diphenylmethyl, p,p′-Dinitrobenzhydryl, 5-Dibenzosuberyl, Triphenylmethyl, α-Naphthyldiphenylmethyl, p-methoxyphenyldiphenylmethyl, Di(p-methoxyphenyl)phenylmethyl, Tri(p-methoxyphenyl)methyl, 4-(4′-Bromophenacyloxy)phenyldiphenylmethyl, 4,4′,4″-Tris(4,5-dichlorophthalimidophenyl)methyl, 4,4′,4″-Tris(levulinoyloxyphenyl)methyl, 4,4′,4″-Tris(benzoyloxyphenyl)methyl, 3-(Imidazol-1-ylmethyl)bis(4′,4″-dimethoxyphenyl)methyl, 1,1-Bis(4-methoxyphenyl)-1′-pyrenylmethyl, 9-Anthryl, 9-(9-Phenyl)xanthenyl, 9-(9-Phenyl-10-oxo)anthryl, 1,3-Benzodithiolan-2-yl, Benzisothiazolyl S,S-Dioxido); Silyl Ethers (Trimethylsilyl, Triethylsilyl, Triisopropylsilyl, Dimethylisopropylsilyl, Diethylisopropylsily, Dimethylthexylsilyl, t-Butyldimethylsilyl, t-Butyldiphenylsilyl, Tribenzylsilyl, Tri-p-xylylsilyl, Triphenylsilyl, Diphenylmethylsilyl, t-Butylmethoxyphenylsilyl); Esters (Formate, Benzoylformate, Acetate, Choroacetate, Dichloroacetate, Trichloroacetate, Trifluoroacetate, Methoxyacetate, Triphenylmethoxyacetate, Phenoxyacetate, p-Chlorophenoxyacetate, p-poly-Phenylacetate, 3-Phenylpropionate, 4-Oxopentanoate (Levulinate), 4,4-(Ethylenedithio)pentanoate, Pivaloate, Adamantoate, Crotonate, 4-Methoxycrotonate, Benzoate, p-Phenylbenzoate, 2,4,6-Trimethylbenzoate (Mesitoate)); Carbonates (Methyl, 9-Fluorenylmethyl, Ethyl, 2,2,2-Trichloroethyl, 2-(Trimethylsilyl)ethyl, 2-(Phenylsulfonyl)ethyl, 2-(Triphenylphosphonio)ethyl, Isobutyl, Vinyl, Allyl, p-Nitrophenyl, Benzyl, p-Methoxybenzyl, 3,4-Dimethoxybenzyl, o-Nitrobenzyl, p-Nitrobenzyl, S-Benzyl Thiocarbonate, 4-Ethoxy-1-naphthyl, Methyl Dithiocarbonate); Groups With Assisted Cleavage (2-Iodobenzoate, 4-Azidobutyrate, 4-Niotro-4-methylpentanoate, o-(Dibromomethyl)benzoate, 2-Formylbenzenesulfonate, 2-(Methylthiomethoxy)ethyl Carbonate, 4-(Methylthiomethoxy)butyrate, 2-(Methylthiomethoxymethyl)benzoate); Miscellaneous Esters (2,6-Dichloro-4-methylphenoxyacetate, 2,6-Dichloro-4-(1,1,3,3 tetramethylbutyl)phenoxyacetate, 2,4-Bis(1,1-dimethylpropyl)phenoxyacetate, Chorodiphenylacetate, Isobutyrate, Monosuccinoate, (E)-2-Methyl-2-butenoate (Tigloate), o-(Methoxycarbonyl)benzoate, p-poly-Benzoate, α-Naphthoate, Nitrate, Alkyl N,N,N′,N′-Tetramethylphosphorodiamidate, N-Phenylcarbamate, Borate, Dimethylphosphinothioyl, 2,4-Dinitrophenylsulfenate); and Sulfonates (Sulfate, Methanesulfonate (Mesylate), Benzylsulfonate, Tosylate).

More typically, R₆ₐ hydroxy protecting groups include substituted methyl ethers, substituted benzyl ethers, silyl ethers, and esters including sulfonic acid esters, still more typically, trialkylsilyl ethers, tosylates and acetates.

Typical 1,2-diol protecting groups (thus, generally where two OH groups are taken together with the R₆ₐ protecting functionality) are described in Greene at pages 118-142 and include Cyclic Acetals and Ketals (Methylene, Ethylidene, 1-t-Butylethylidene, 1-Phenylethylidene, (4-Methoxyphenyl)ethylidene, 2,2,2-Trichloroethylidene, Acetonide (Isopropylidene), Cyclopentylidene, Cyclohexylidene, Cycloheptylidene, Benzylidene, p-Methoxybenzylidene, 2,4-Dimethoxybenzylidene, 3,4-Dimethoxybenzylidene, 2-Nitrobenzylidene); Cyclic Ortho Esters (Methoxymethylene, Ethoxymethylene, Dimethoxymethylene, 1-Methoxyethylidene, 1-Ethoxyethylidine, 1,2-Dimethoxyethylidene, α-Methoxybenzylidene, 1-(N,N-Dimethylamino)ethylidene Derivative, α-(N,N-Dimethylamino)benzylidene Derivative, 2-Oxacyclopentylidene); Silyl Derivatives (Di-t-butylsilylene Group, 1,3-(1,1,3,3 Tetraisopropyldisiloxanylidene), and Tetra-t-butoxydisiloxane-1,3-diylidene), Cyclic Carbonates, Cyclic Boronates, Ethyl Boronate and Phenyl Boronate.

More typically, 1,2-diol protecting groups include those shown in Table B, still more typically, epoxides, acetonides, cyclic ketals and aryl acetals.

TABLE B

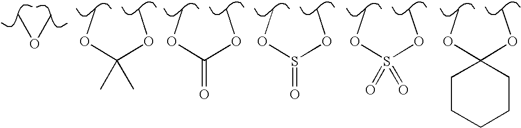

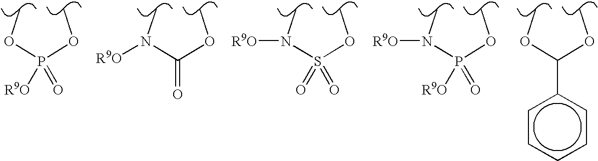

wherein R⁹ is C₁-C₆ alkyl.

R_{6b} is H, a protecting group for amino or the residue of a carboxyl-containing compound, in particular H, —C(O)R₄, an amino acid, a polypeptide or a protecting group not —C(O)R₄, amino acid or polypeptide. Amide-forming R_{6b} are found for instance in group G₁. When R_{6b} is an amino acid or polypeptide it has the structure R₁₅NHCH(Rl6)C(O)—, where R₁₅ is H, an amino acid or polypeptide residue, or R₅, and R₁₆ is defined below.

R₁₆ is lower alkyl or lower alkyl (C₁-C₆) substituted with amino, carboxyl, amide, carboxyl ester, hydroxyl, C₆-C₇ aryl, guanidinyl, imidazolyl, indolyl, sulffhydryl, sulfoxide, and/or alkylphosphate. R₁₆ also is taken together with the amino acid a N to form a proline residue (R₁₆═—CH₂)₃—). However, R₁₆ is generally the side group of a naturally-occurring amino acid such as H, —CH₃, —CH(CH₃)₂, —CH₂—CH(CH₃)₂, —CHCH₃—CH₂—CH₃, —CH₂—C₆H₅, —CH₂CH₂—S—CH₃, —CH₂OH, —CH(OH)—CH₃, —CH₂—SH, —CH₂—C₆H₄OH, —CH₂—CO—NH₂, —CH₂—CH₂—CO—NH₂, —CH₂—COOH, —CH₂—CH₂—COOH, —(CH₂)₄—NH₂ and —(CH₂)₃—NH—C(NH₂)—NH₂. R₁₆ also includes 1-guanidinoprop-3-yl, benzyl, 4-hydroxybenzyl, imidazol-4-yl, indol-3-yl, methoxyphenyl and ethoxyphenyl.

R_{6b} are residues of carboxylic acids for the most part, but any of the typical amino protecting groups described by Greene at pages 315-385 are useful. They include Carbamates (methyl and ethyl, 9-fluorenylmethyl, 9(2-sulfo)fluoroenylmethyl, 9-(2,7-dibromo)fluorenylmethyl, 2,7-di-t-buthyl-[9-(10,10-dioxo-10,10,10,10-tetrahydrothioxanthyl)]methyl, 4-methoxyphenacyl); Substituted Ethyl (2,2,2-trichoroethyl, 2-trimethylsilylethyl, 2-phenylethyl, 1-(1-adamantyl)-1-methylethyl, 1,1-dimethyl-2-haloethyl, 1,1-dimethyl-2,2-dibromoethyl, 1,1-dimethyl-2,2,2-trichloroethyl, 1-methyl-1-(4-biphenylyl)ethyl, 1-(3,5-di-t-butylphenyl)-1-methylethyl, 2-(2′- and 4′-pyridyl)ethyl, 2-(N,N-dicyclohexylcarboxamido)ethyl, t-butyl, 1-adamantyl, vinyl, allyl, 1-isopropylallyl, cinnamyl, 4-nitrocinnamyl, 8-quinolyl, N-hydroxypiperidinyl, alkyldithio, benzyl, p-methoxybenzyl, p-nitrobenzyl, p-bromobenzyl, p-chorobenzyl, 2,4-dichlorobenzyl, 4-methylsulfinylbenzyl, 9-anthrylmethyl, diphenylmethyl); Groups With Assisted Cleavage (2-methylthioethyl, 2-methylsulfonylethyl, 2-(p-toluenesulfonyl)ethyl, [2-(1,3-dithianyl)]methyl, 4-methylthiophenyl, 2,4-dimethylthiophenyl, 2-phosphonioethyl, 2-triphenylphosphonioisopropyl, 1,1-dimethyl-2-cyanoethyl, m-choro-p-acyloxybenzyl, p-(dihydroxyboryl)benzyl, 5-benzisoxazolylmethyl, 2-(trifluoromethyl)-6-chromonylmethyl); Groups Capable of Photolytic Cleavage (m-nitrophenyl, 3,5-dimethoxybenzyl, o-nitrobenzyl, 3,4-dimethoxy-6-nitrobenzyl, phenyl(o-nitrophenyl)methyl); Urea-Type Derivatives (phenothiazinyl-(10)-carbonyl, N′-p-toluenesulfonylaminocarbonyl, N′-phenylaminothiocarbonyl); Miscellaneous Carbamates (t-amyl, S-benzyl thiocarbamate, p-cyanobenzyl, cyclobutyl, cyclohexyl, cyclopentyl, cyclopropylmethyl, p-decyloxybenzyl, diisopropylmethyl, 2,2-dimethoxycarbonylvinyl, o-(N,N-dimethylcarboxamido)benzyl, 1,1-dimethyl-3-(N,N-dimethylcarboxamido)propyl, 1,1-dimethylpropynyl, di(2-pyridyl)methyl, 2-furanylmethyl, 2-lodoethyl, Isobornyl, Isobutyl, Isonicotinyl, p-(p′-Methoxyphenylazo)benzyl, 1-methylcyclobutyl, 1-methylcyclohexyl, 1-methyl-1-cyclopropylmethyl, 1-methyl-1-(3,5-dimethoxyphenyl)ethyl, 1-methyl-1-(p-phenylazophenyl)ethyl, 1-methyl-1-phenylethyl, 1-methyl-1-(4-pyridyl)ethyl, phenyl, p-(phenylazo)benzyl, 2,4,6-tri-t-butylphenyl, 4-(trimethylammonium)benzyl, 2,4,6-trimethylbenzyl); Amides (N-formyl, N-acetyl, N-choroacetyl, N-trichoroacetyl, N-trifluoroacetyl, N-phenylacetyl, N-3-phenylpropionyl, N-picolinoyl, N-3-pyridylcarboxamide, N-benzoylphenylalanyl, N-benzoyl, N-p-phenylbenzoyl); Amides With Assisted Cleavage (N-o-nitrophenylacetyl, N-o-nitrophenoxyacetyl, N-acetoacetyl, (N′-dithiobenzyloxycarbonylamino)acetyl, N-3-(p-hydroxyphenyl)propionyl, N-3-(o-nitrophenyl)propionyl, N-2-methyl-2-(o-nitrophenoxy)propionyl, N-2-methyl-2-(o-phenylazophenoxy)propionyl, N-4-chlorobutyryl, N-3-methyl-3-nitrobutyryl, N-o-nitrocinnamoyl, N-acetylmethionine, N-o-nitrobenzoyl, N-o-(benzoyloxymethyl)benzoyl, 4,5-diphenyl-3-oxazolin-2-one); Cyclic Imide Derivatives (N-phthalimide, N-dithiasuccinoyl, N-2,3-diphenylmaleoyl, N-2,5-dimethylpyrrolyl, N-1,1,4,4-tetramethyldisilylazacyclopentane adduct, 5-substituted 1,3-dimethyl-1,3,5-triazacyclohexan-2-one, 5-substituted 1,3-dibenzyl-1,3-5-triazacyclohexan-2-one, 1-substituted 3,5-dinitro-4-pyridonyl); N-Alkyl and N-Aryl Amines (N-methyl, N-allyl, N-[2-(trimethylsilyl)ethoxy]methyl, N-3-acetoxypropyl, N-(1-isopropyl-4-nitro-2-oxo-3-pyrrolin-3-yl), Quaternary Ammonium Salts, N-benzyl, N-di(4-methoxyphenyl)methyl, N-5-dibenzosuberyl, N-triphenylmethyl, N-(4-methoxyphenyl)diphenylmethyl, N-9-phenylfluorenyl, N-2,7-dichloro-9-fluorenylmethylene, N-ferrocenylmethyl, N-2-picolylamine N′-oxide), Imine Derivatives (N-1,1-dimethylthiomethylene, N-benzylidene, N-p-methoxybenylidene, N-diphenylmethylene, N-[(2-pyridyl)mesityl]methylene, N,(N′,N′-dimethylaminomethylene, N,N′-isopropylidene, N-p-nitrobenzylidene, N-salicylidene, N-5-chlorosalicylidene, N-(5-chloro-2-hydroxyphenyl)phenylmethylene, N-cyclohexylidene); Enamine Derivatives (N-(5,5-dimethyl-3-oxo-1-cyclohexenyl)); N-Metal Derivatives (N-borane derivatives, N-diphenylborinic acid derivatives, N-[phenyl(pentacarbonylchromium- or -tungsten)]carbenyl, N-copper or N-zinc chelate); N—N Derivatives (N-nitro, N-nitroso, N-oxide); N—P Derivatives (N-diphenylphosphinyl, N-dimethylthiophosphinyl, N-diphenylthiophosphinyl, N-dialkyl phosphoryl, N-dibenzyl phosphoryl, N-diphenyl phosphoryl); N—Si Derivatives; N—S Derivatives; N-Sulfenyl Derivatives (N-benzenesulfenyl, N-o-nitrobenzenesulfenyl, N-2,4-dinitrobenzenesulfenyl, N-pentachlorobenzenesulfenyl, N-2-nitro-4-methoxybenzenesulfenyl, N-triphenylmethylsulfenyl, N-3-nitropyridinesulfenyl); and N-sulfonyl Derivatives (N-p-toluenesulfonyl, N-benzenesulfonyl, N-2,3,6-trimethyl-4-methoxybenzenesulfonyl, N-2,4,6-trimethoxybenzenesulfonyl, N-2,6-dimethyl-4-methoxybenzenesulfonyl, N-pentamethylbenzenesulfonyl, N-2,3,5,6,-tetramethyl-4-methoxybenzenesulfonyl, N-4-methoxybenzenesulfonyl, N-2,4,6-trimethylbenzenesulfonyl, N-2,6-dimethoxy-4-methylbenzenesulfonyl, N-2,2,5,7,8-pentamethylchroman-6-sulfonyl, N-methanesulfonyl, N-β-trimethylsilyethanesulfonyl, N-9-anthracenesulfonyl, N-4-(4′,8′-dimethoxynaphthylmethyl)benzenesulfonyl, N-benzylsulfonyl, N-trifluoromethylsulfonyl, N-phenacylsulfonyl).

More typically, protected amino groups include carbamates and amides, still more typically, —NHC(O)R₁ or —N═CRlN(R₁)₂. Another protecting group, also usefull as a prodrug at the G₁ site, particularly for amino or —NH(R₅), is:

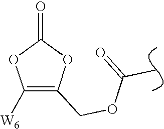

see for example Alexander, J. et al., “J. Med. Chem.” 39:480-486 (1996).

R_{6c} is H or the residue of an amino-containing compound, in particular an amino acid, a polypeptide, a protecting group, —NHSO₂R₄, NHC(O)R₄, —N(R₄)₂, NH₂ or —NH(R₄)(H), whereby for example the carboxyl or phosphonic acid groups of W₁ are reacted with the amine to form an amide, as in —C(O)R_{6c}, —P(O)(R_{6c})₂ or —P(O)(OH)(R_{6c}). In general, R_{6c} has the structure R₁₇C(O)CH(Rl₆)NH—, where R₁₇ is OH, OR₆ₐ, OR₅, an amino acid or a polypeptide residue.

Amino acids are low molecular weight compounds, on the order of less than about 1,000 MW, that contain at least one amino or imino group and at least one carboxyl group. Generally the amino acids will be found in nature, i.e., can be detected in biological material such as bacteria or other microbes, plants, animals or man. Suitable amino acids typically are alpha amino acids, i.e. compounds characterized by one amino or imino nitrogen atom separated from the carbon atom of one carboxyl group by a single substituted or unsubstituted alpha carbon atom. Of particular interest are hydrophobic residues such as mono-or di-alkyl or aryl amino acids, cycloalkylamino acids and the like. These residues contribute to cell permeability by increasing the partition coefficient of the parental drug. Typically, the residue does not contain a sulfhydryl or guanidino substituent.

Naturally-occurring amino acid residues are those residues found naturally in plants, animals or microbes, especially proteins thereof. Polypeptides most typically will be substantially composed of such naturally-occurring amino acid residues. These amino acids are glycine, alanine, valine, leucine, isoleucine, serine, threonine, cysteine, methionine, glutamic acid, aspartic acid, lysine, hydroxylysine, arginine, histidine, phenylalanine, tyrosine, tryptophan, proline, asparagine, glutamine and hydroxyproline.

When R_{6b} and R_{6c} are single amino acid residues or polypeptides they usually are substituted at R₃, W₆, W₁ and/or W₂, but typically only W₁ or W₂. These conjugates are produced by forming an amide bond between a carboxyl group of the amino acid (or C-terminal amino acid of a polypeptide for example) and W₂. Similarly, conjugates are formed between W₁ and an amino group of an amino acid or polypeptide. Generally, only one of any site in the parental molecule is amidated with an amino acid as described herein, although it is within the scope of this invention to introduce amino acids at more than one permitted site. Usually, a carboxyl group of W₁ is amidated with an amino acid. In general, the α-amino or α-carboxyl group of the amino acid or the terminal amino or carboxyl group of a polypeptide are bonded to the parental functionalities, i.e., carboxyl or amino groups in the amino acid side chains generally are not used to form the amide bonds with the parental compound (although these groups may need to be protected during synthesis of the conjugates as described further below).

With respect to the carboxyl-containing side chains of amino acids or polypeptides it will be understood that the carboxyl group optionally will be blocked, e.g. by R₆ₐ, esterified with R₅ or amidated with R_{6c}. Similarly, the amino side chains R₁₆ optionally will be blocked with R_{6b} or substituted with R₅.

Such ester or amide bonds with side chain amino or carboxyl groups, like the esters or amides with the parental molecule, optionally are hydrolyzable in vivo or in vitro under acidic (pH<3) or basic (pH>10) conditions. Alternatively, they are substantially stable in the gastrointestinal tract of humans but are hydrolyzed enzymatically in blood or in intracellular environments. The esters or amino acid or polypeptide amidates also are useful as intermediates for the preparation of the parental molecule containing free amino or carboxyl groups. The free acid or base of the parental compound, for example, is readily formed from the esters or amino acid or polypeptide conjugates of this invention by conventional hydrolysis procedures.

When an amino acid residue contains one or more chiral centers, any of the D, L, meso, threo or erythro (as appropriate) racemates, scalemates or mixtures thereof may be used. In general, if the intermediates are to be hydrolyzed non-enzymatically (as would be the case where the amides are used as chemical intermediates for the free acids or free amines), D isomers are useful. On the other hand, L isomers are more versatile since they can be susceptible to both non-enzymatic and enzymatic hydrolysis, and are more efficiently transported by amino acid or dipeptidyl transport systems in the gastrointestinal tract.

Examples of suitable amino acids whose residues are represented by R_{6b}and R_{6c} include the following:

Glycine;

Aminopolycarboxylic acids, e.g., aspartic acid, β-hydroxyaspartic acid, glutamic acid, β-hydroxyglutamic acid, β-methylaspartic acid, β-methylglutamic acid, β,β-dimethylaspartic acid, γ-hydroxyglutamic acid, β,γ-dihydroxyglutamic acid, β-phenylglutamic acid, γ-methyleneglutamic acid, 3-aminoadipic acid, 2-aminopimelic acid, 2-aminosuberic acid and 2-aminosebacic acid;

Amino acid amides such as glutamine and asparagine;

Polyamino- or polybasic-monocarboxylic acids such as arginine, lysine, β-aminoalanine, γ-aminobutyrine, ornithine, citruline, homoarginine, homocitrulline, hydroxylysine, allohydroxylsine and diaminobutyric acid;

Other basic amino acid residues such as histidine;

Diaminodicarboxylic acids such as α,α′-diaminosuccinic acid, α,α′-diaminoglutaric acid, α,α′-diaminoadipic acid, α,α′-diaminopimelic acid, α,α′-diamino-β-hydroxypimelic acid, α,α′-diaminosuberic acid, α,α′-diaminoazelaic acid, and α,α′-diaminosebacic acid;

Imino acids such as proline, hydroxyproline, allohydroxyproline, γ-methylproline, pipecolic acid, 5-hydroxypipecolic acid, and azetidine-2-carboxylic acid;

A mono- or di-alkyl (typically C₁-C₈ branched or normal) amino acid such as alanine, valine, leucine, allylglycine, butyrine, norvaline, norleucine, heptyline, α-methylserine, α-amino-α-methyl-γ-hydroxyvaleric acid, α-amino-α-methyl-δ-hydroxyvaleric acid, α-amino-α-methyl-ε-hydroxycaproic acid, isovaline, α-methylglutamic acid, α-aminoisobutyric acid, α-aminodiethylacetic acid, α-aminodiisopropylacetic acid, α-aminodi-n-propylacetic acid, α-aminodiisobutylacetic acid, α-aminodi-n-butylacetic acid, α-aminoethylisopropylacetic acid, α-amino-n-propylacetic acid, α-aminodiisoamyacetic acid, α-methylaspartic acid, α-methylglutamic acid, 1-aminocyclopropane-1-carboxylic acid, isoleucine, alloisoleucine, tert-leucine, β-methyltryptophan and α-amino-β-ethyl-β-phenylpropionic acid;

β-phenylserinyl;

Aliphatic α-amino-β-hydroxy acids such as serine, β-hydroxyleucine, β-hydroxynorleucine, β-hydroxynorvaline, and α-amino-β-hydroxystearic acid;

α-Amino, α-, γ-, δ- or ε-hydroxy acids such as homoserine, γ-hydroxynorvaline, δ-hydroxynorvaline and epsilon-hydroxynorleucine residues; canavine and canaline; γ-hydroxyornithine;

2-hexosaminic acids such as D-glucosaminic acid or D-galactosaminic acid;

α-Amino-β-thiols such as penicillamine, β-thiolnorvaline or β-thiolbutyrine;

Other sulfur containing amino acid residues including cysteine; homocystine, β-phenylmethionine, methionine, S-allyl-L-cysteine sulfoxide, 2-thiolhistidine, cystathionine, and thiol ethers of cysteine or homocysteine;

Phenylalanine, tryptophan and ring-substituted αamino acids such as the phenyl- or cyclohexylamino acids α-aminophenylacetic acid, α-aminocyclohexylacetic acid and α-amino-β-cyclohexylpropionic acid; phenylalanine analogues and derivatives comprising aryl, lower alkyl, hydroxy, guanidino, oxyalkylether, nitro, sulfur or halo-substituted phenyl (e.g., tyrosine, methyltyrosine and o-chloro-, p-chloro-, 3,4-dicloro, o-, m- or p-methyl-, 2,4,6-trimethyl-, 2-ethoxy-5-nitro-, 2-hydroxy-5-nitro- and p-nitro-phenylalanine); furyl-, thienyl-, pyridyl-, pyrimidinyl-, purinyl- or naphthyl-alanines; and tryptophan analogues and derivatives including kynurenine, 3-hydroxykynurenine, 2-hydroxytryptophan and 4-carboxytryptophan;

α-Amino substituted amino acids including sarcosine (N-methylglycine), N-benzylglycine, N-methylalanine, N-benzylalanine, N-methylphenylalanine, N-benzylphenylalanine, N-methylvaline and N-benzylvaline; and

α-Hydroxy and substituted α-hydroxy amino acids including serine, threonine, allothreonine, phosphoserine and phosphothreonine.

Polypeptides are polymers of amino acids in which a carboxyl group of one amino acid monomer is bonded to an amino or imino group of the next amino acid monomer by an amide bond. Polypeptides include dipeptides, low molecular weight polypeptides (about 1500-5000 MW) and proteins. Proteins optionally contain 3, 5, 10, 50, 75, 100 or more residues, and suitably are substantially sequence-homologous with human, animal, plant or microbial proteins. They include enzymes (e.g., hydrogen peroxidase) as well as immunogens such as KLH, or antibodies or proteins of any type against which one wishes to raise an immune response. The nature and identity of the polypeptide may vary widely.

The polypeptide amidates are useful as immunogens in raising antibodies against either the polypeptide (if it is not immunogenic in the animal to which it is administered) or against the epitopes on the remainder of the compound of this invention.

Antibodies capable of binding to the parental non-peptidyl compound are used to separate the parental compound from mixtures, for example in diagnosis or manufacturing of the parental compound. The conjugates of parental compound and polypeptide generally are more immunogenic than the polypeptides in closely homologous animals, and therefore make the polypeptide more immunogenic for facilitating raising antibodies against it. Accordingly, the polypeptide or protein may not need to be immunogenic in an animal typically used to raise antibodies, e.g., rabbit, mouse, horse, or rat, but the final product conjugate should be immunogenic in at least one of such animals. The polypeptide optionally contains a peptidolytic enzyme cleavage site at the peptide bond between the first and second residues adjacent to the acidic heteroatom. Such cleavage sites are flanked by enzymatic recognition structures, e.g. a particular sequence of residues recognized by a peptidolytic enzyme.

Peptidolytic enzymes for cleaving the polypeptide conjugates of this invention are well known, and in particular include carboxypeptidases. Carboxypeptidases digest polypeptides by removing C-terminal residues, and are specific in many instances for particular C-terminal sequences. Such enzymes and their substrate requirements in general are well known. For example, a dipeptide (having a given pair of residues and a free carboxyl terminus) is covalently bonded through its ax-amino group to the phosphorus or carbon atoms of the compounds herein. In embodiments where W₁ is phosphonate it is expected that this peptide will be cleaved by the appropriate peptidolytic enzyme, leaving the carboxyl of the proximal amino acid residue to autocatalytically cleave the phosphonoamidate bond.

Suitable dipeptidyl groups (designated by their single letter code) are AA, AR, AN, AD, AC, AE, AQ, AG, AH, AI, AL, AK, AM, AF, AP, AS, AT, AW, AY, AV, RA, RR, RN, RD, RC, RE, RQ, RG, RH, RI, RL, RK, RM, RF, RP, RS, RT, RW, RY, RV, NA, NR, NN, ND, NC, NE, NQ, NG, NH, NI, NL, NK, NM, NF, NP, NS, NT, NW, NY, NV, DA, DR, DN, DD, DC, DE, DQ, DG, DH, DI, DL, DK, DM, DF, DP, DS, DT, DW, DY, DV, CA, CR, CN, CD, CC, CE, CQ, CG, CH, CI, CL, CK, CM, CF, CP, CS, CT, CW, CY, CV, EA, ER, EN, ED, EC, EE, EQ, EG, EH, EI, EL, EK, EM, EF, EP, ES, ET, EW, EY, EV, QA, QR, QN, QD, QC, QE, QQ, QG, QH, QI, QL, QK, QM, QF, QP, QS, QT, QW, QY, QV, GA, GR, GN, GD, GC, GE, GQ, GG, GH, GI, GL, GK, GM, GF, GP, GS, GT, GW, GY, GV, HA, HR, HN, HD, HC, HE, HQ, HG, HH, HI, HL, HK, HM, HF, HP, HS, HT, HW, HY, HV, IA, IR, IN, ID, IC, IE, IQ, IG, IH, II, IL, IK, IM, IF, IP, IS, IT, IW, IY, IV, LA, LR, LN, LD, LC, LE, LQ, LG, LH, LI, LL, LK, LM, LF, LP, LS, LT, LW, LY, LV, KA, KR, KN, KD, KC, KE, KQ KG, KH, KI, KL, KK, KM, KF, KP, KS, KT, KW, KY, KV, MA, MR, MN, MD, MC, ME, MQ MG, MH, MI, ML, MK, MM, MF, MP, MS, MT, MW, MY, MV, FA, FR, FN, FD, FC, FE, FQ, FG, FH, FI, FL, FK, FM, FF, FP, FS, FT, FW, FY, FV, PA, PR, PN, PD, PC, PE, PQ, PG, PH, PI, PL, PK, PM, PF, PP, PS, PT, PW, PY, PV, SA, SR, SN, SD, SC, SE, SQ, SG, SH, SI, SL, SK, SM, SF, SP, SS, ST, SW, SY, SV, TA, TR, TN, TD, TC, TE, TQ, TG, TH, TI, TL, TK, TM, TF, TP, TS, TT, TW, TY, TV, WA, WR, WN, WD, WC, WE, WQ, WG, WH, WI, WL, WK, WM, WF, WP, WS, WT, WW, WY, WV, YA, YR, YN, YD, YC, YE, YQ, YG, YH, YI, YL, YK, YM, YF, YP, YS, YT, YW, YY, YV, VA, VR, VN, VD, VC, VE, VQ, VG, VH, VI, VL, VK, VM, VF, VP, VS, VT, VW, VY and VV.

Tripeptide residues are also useful as R_{6b} or R_{6c}. When W₁ is phosphonate, the sequence —X₄-pro-X₅— (where X₄ is any amino acid residue and X₅ is an amino acid residue, a carboxyl ester of proline, or hydrogen) will be cleaved by luminal carboxypeptidase to yield X₄ with a free carboxyl, which in turn is expected to autocatalytically cleave the phosphonoamidate bond. The carboxy group of X₅ optionally is esterified with benzyl.

Dipeptide or tripeptide species can be selected on the basis of known transport properties and/or susceptibility to peptidases that can affect transport to intestinal mucosal or other cell types. Dipeptides and tripeptides lacking an α-amino group are transport substrates for the peptide transporter found in brush border membrane of intestinal mucosal cells (Bai, J. P. F., “Pharm Res.” 9:969-978 (1992). Transport competent peptides can thus be used to enhance bioavailability of the amidate compounds. Di- or tripeptides having one or more amino acids in the D configuration are also compatible with peptide transport and can be utilized in the amidate compounds of this invention. Amino acids in the D configuration can be used to reduce the susceptibility of a di- or tripeptide to hydrolysis by proteases common to the brush border such as aminopeptidase N (EC 3.4.11.2). In addition, di- or tripeptides alternatively are selected on the basis of their relative resistance to hydrolysis by proteases found in the lumen of the intestine. For example, tripeptides or polypeptides lacking asp and/or glu are poor substrates for aminopeptidase A (EC 3.4.11.7), di- or tripeptides lacking amino acid residues on the N-terminal side of hydrophobic amino acids (leu, tyr, phe, val, trp) are poor substrates for endopeptidase 24.11 (EC 3.4.24.11), and peptides lacking a pro residue at the penultimate position at a free carboxyl terminus are poor substrates for carboxypeptidase P (EC 3.4.17). Similar considerations can also be applied to the selection of peptides that are either relatively resistant or relatively susceptible to hydrolysis by cytosolic, renal, hepatic, serum or other peptidases. Such poorly cleaved polypeptide amidates are immunogens or are useful for bonding to proteins in order to prepare immunogens.

Another embodiment of the invention relates to compositions of the formula (VII) or (VIII):

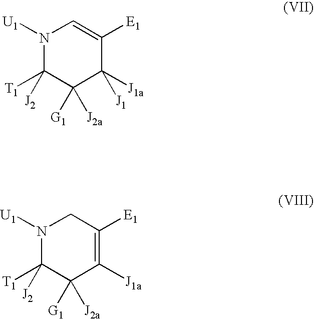

wherein E₁, G₁, T₁, U₁, J₁, J₁ₐ, J₂ and J₂ₐ are as defined above except:

T₁ is —NR₁W₃, a heterocycle, or is taken together with G₁ to form a group having the structure

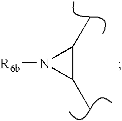

and

X₁ is a bond, —O—, —N(H)—, —N(R₅)—, —S—, —SO—, or —SO₂—; and provided, however, that compounds are excluded wherein U₁ is H or —CH₂CH(OH)CH₂(OH);

and the salts, solvates, resolved enantiomers and purified diastereomers thereof.

Each of the typical or ordinary embodiments of formula (I)-(VI) detailed above are also typical embodiments of formula (VII) and (VIII).

The synthesis of a number of compounds of the formula (VII) and (VIII) wherein U₁ is H or —CH₂CH(OH)CH₂(OH) are provided in Nishimura, Y. et al., “J. Antibiotics” 46(2):300; 46(12):1883 (1993); and “Nat. Prod. Lett.”, 1(1):39 (1992). Attachment of U₁ groups of the present invention proceed as described therein.

### Stereoisomers

The compounds of the invention are enriched or resolved optical isomers at any or all asymmetric atoms. For example, the chiral centers apparent from the depictions are provided as the chiral isomers or racemic mixtures. Both racemic and diasteromeric mixtures, as well as the individual optical isomers isolated or synthesized, substantially free of their enantiomeric or diastereomeric partners, are all within the scope of the invention.

One or more of the following enumerated methods are used to prepare the enantiomerically enriched or pure isomers herein. The methods are listed in approximately their order of preference, i.e., one ordinarily should employ stereospecific synthesis from chiral precursors before chromatographic resolution before spontaneous crystallization.

Stereospecific synthesis is described in the examples. Methods of this type conveniently are used when the appropriate chiral starting material is available and reaction steps are chosen do not result in undesired racemization at chiral sites. One advantage of stereospecific synthesis is that it does not produce undesired enantiomers that must be removed from the final product, thereby lowering overall synthetic yield. In general, those skilled in the art would understand what starting materials and reaction conditions should be used to obtain the desired enantiomerically enriched or pure isomers by stereospecific synthesis. If an unexpected racemization occurs in a method thought to be stereospecific then one needs only to use one of the following separation methods to obtain the desired product.

If a suitable stereospecific synthesis cannot be empirically designed or determined with routine experimentation then those skilled in the art would turn to other methods. One method of general utility is chromotographic resolution of enantiomers on chiral chromatography resins. These resins are packed in columns, commonly called Pirkle columns, and are commercially available. The columns contain a chiral stationary phase. The racemate is placed in solution and loaded onto the column, and thereafter separated by HPLC. See for example, Proceedings Chromatographic Society—International Symposium on Chiral Separations, Sept. 3-4, 1987. Examples of chiral columns that could be used to screen for the optimal separation technique would include Diacel Chriacel OD, Regis Pirkle Covalent Dphenylglycine, Regis Pirkle Type 1A, Astec Cyclobond II, Astec Cyclobond III, Serva Chiral D-DL=Daltosil 100, Bakerbond DNBLeu, Sumipax OA-1000, Merck Cellulose Triacetate column, Astec Cyclobond I-Beta, or Regis Pirkle Covalent D-Naphthylalanine. Not all of these columns are likely to be effective with every racemic mixture. However, those skilled in the art understand that a certain amount of routine screening may be required to identify the most effective stationary phase. When using such columns it is desireable to employ embodiments of the compounds of this invention in which the charges are not neutralized, e.g., where acidic functionalities such as carboxyl are not esterified or amidated.

Another method entails converting the enantiomers in the mixture to diasteriomers with chiral auxiliaries and then separting the conjugates by ordinary column chromatography. This is a very suitable method, particularly when the embodiment contains free carboxyl, amino or hydroxyl that will form a salt or covalent bond to a chiral auxiliary. Chirally pure amino acids, organic acids or organosulfonic acids are all worthwhile exploring as chiral auxiliaries, all of which are well known in the art. Salts with such auxiliaries can be formed, or they can be covalently (but reversibly) bonded to the functional group. For example, pure D or L amino acids can be used to amidate the carboxyl group of embodiments of this invention and then separated by chromatography.

Enzymatic resolution is another method of potential value. In such methods one prepares covalent derivatives of the enantiomers in the racemic mixture, generally lower alkyl esters (for example of carboxyl), and then exposes the derivative to enzymatic cleavage, generally hydrolysis. For this method to be successful an enzyme must be chosen that is capable of stereospecific cleavage, so it is frequently necessary to routinely screen several enzymes. If esters are to be cleaved, then one selects a group of esterases, phosphatases, and lipases and determines their activity on the derivative. Typical esterases are from liver, pancreas or other animal organs, and include porcine liver esterase.

If the enatiomeric mixture separates from solution or a melt as a conglomerate, i.e., a mixture of enantiomerically-pure crystals, then the crystals can be mechanically separated, thereby producing the enantiomerically enriched preparation. This method, however, is not practical for large scale preparations and is of no value for true racemic compounds.

Asymmetric synthesis is another technique for achieving enantiomeric enrichment. For example, a chiral protecting group is reacted with the group to be protected and the reaction mixture allowed to equilibrate. If the reaction is enantiomerically specific then the product will be enriched in that enantiomer.

Further guidance in the separation of enantiomeric mixtures can be found, by way of example and not limitation, in “Enantiomers, Racemates, and resolutions”, Jean Jacques, Andre Collet, and Samuel H. Wilen (Krieger Publishing Company, Malabar, Fla., 1991, ISBN 0-89464-618-4). In particular, Part 2, Resolution of Enantiomer Mixture, pages 217-435; more particularly, section 4, Resolution by Direct Crystallization, pages 217-251, section 5, Formation and Separation of Diastereomers, pages 251-369, section 6, Crystallization-Induced Asymmetric Transformations, pages 369-378, and section 7, Experimental Aspects and Art of Resolutions, pages 378-435; still more particularly, section 5.1.4, Resolution of Alcohols, Transformation of Alcohols into Salt-Forming Derivatives, pages 263-266, section 5.2.3, Covalent Derivatives of Alcohols, Thiols, and Phenols, pages 332-335, section 5.1.1, Resolution of Acids, pages 257-259, section 5.1.2, Resolution of Bases, pages 259-260, section 5.1.3, Resolution of Amino Acids, page 261-263, section 5.2.1, Covalent Derivatives of Acids, page 329, section 5.2.2, Covalent derivatives of Amines, pages 330-331, section 5.2.4, Covalent Derivatives of Aldehydes, Ketones, and Sulfoxides, pages 335-339, and section 5.2.7, Chromatographic Behavior of Covalent Diastereomers, pages 348-354, are cited as examples of the skill of the art.

Exemplary stereochemistry of the compounds of this invention is set forth below in Table C.

TABLE C

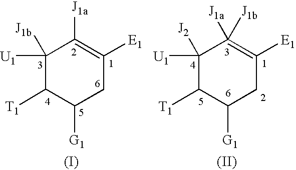

Formula (I)

| E₁ | J₁ₐ | J_{1b} | U₁ | T₁ | G₁ |
|---|---|---|---|---|---|
| — | — | α | β | α | α |
| — | — | β | α | α | α |
| — | — | α | β | β | α |
| — | — | α | β | α | β |
| — | — | β | α | β | α |
| — | — | β | α | α | β |
| — | — | α | β | β | β |
| — | — | β | α | β | β |

Formula (I)

| E₁ | J₁ₐ | J_{1b} | J₂ | U₁ | T₁ | G₁ |
|---|---|---|---|---|---|---|
| — | α | β | α | β | α | α |
| — | β | α | α | β | α | α |
| — | α | β | β | α | α | α |
| — | α | β | α | β | β | α |
| — | α | β | α | β | α | β |
| — | β | α | β | α | α | α |
| — | β | α | α | β | β | α |
| — | β | α | α | β | α | β |
| — | α | β | β | α | β | α |
| — | α | β | β | α | α | β |
| — | α | β | α | β | β | β |
| — | β | α | β | α | β | α |
| — | β | α | β | β | α | β |
| — | β | α | α | β | β | β |
| — | α | β | β | α | β | β |
| — | β | α | β | α | β | β |

The compounds of the invention can also exist as tautomeric isomers in certain cases. For example, ene-amine tautomers can exist for imidazole, guanidine, amidine, and tetrazole systems and all their possible tautomeric forms are within the scope of the invention.

### Exemplary Enumerated Compound

By way of example and not limitation, embodiment compounds are named below in tabular format (Table 6). Generally, each compound is depicted as a substituted nucleus in which the nucleus is designated by capital letter and each substituent is designated in order by lower case letter or number. Tables 1a 1b are a schedule of nuclei which differ principally by the position of ring unsaturation and the nature of ring substituents. Each nucleus is given a alphabetical designation from Tables 1a and 1b, and this designation appears first in each compound name. Similarly, Tables 2a-av, 3a-b, 4a-c, and 5a-d list the selected Q₁, Q₂, Q₃ and Q₄ substituents, again by letter or number designation. Accordingly, each named compound will be depicted by a capital letter designating the nucleus from Table 1a-1b, followed by a by a capital letter designating the nucleus from Table 1a-1b, followed by a substituent, a number designating the Q₃ substituent, and a lower case letter or letters designating the Q₄ substituent. Thus, structure 8, scheme 1, is represented by A.49.a.4.i. Q₁-Q₄, it should be understood, do not represent groups or atoms but are simply connectivity designations.

TABLE 1a

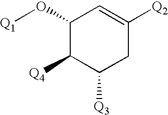

A

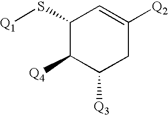

B

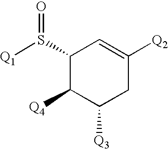

C

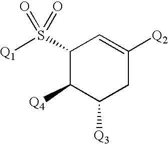

D

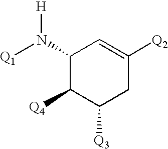

E

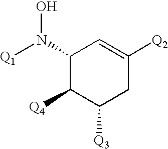

F

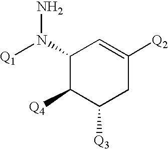

G

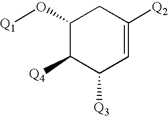

H

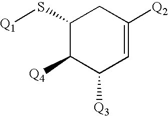

I

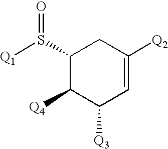

J

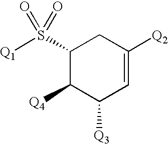

K

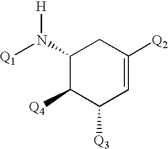

L

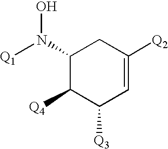

M

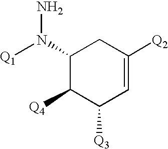

N

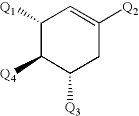

O

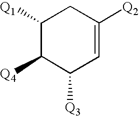

P

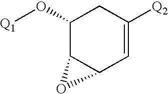

Q

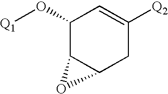

R

TABLE 1b

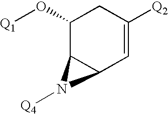

S

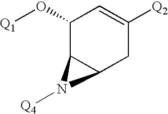

T

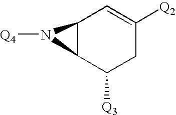

U

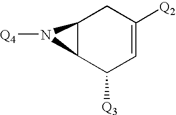

V

TABLE 2a

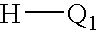

1

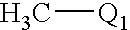

2

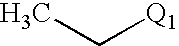

3

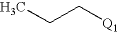

4

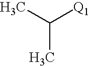

5

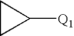

6

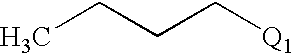

7

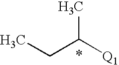

8

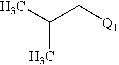

9

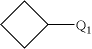

10

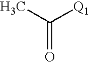

11

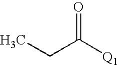

12

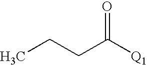

13

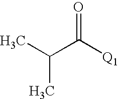

14

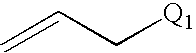

15

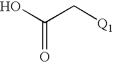

16

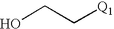

17

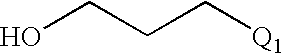

18

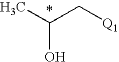

19

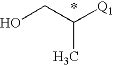

20

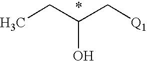

21

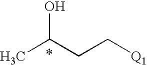

22

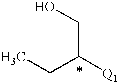

23

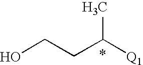

24

TABLE 2b

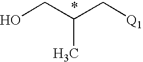

25

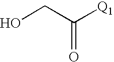

26

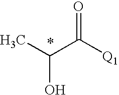

27

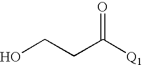

28

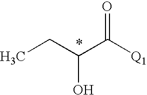

29

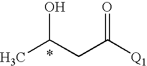

30

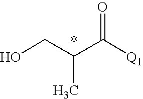

31

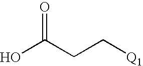

32

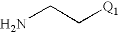

33

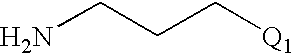

34

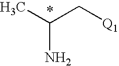

35

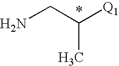

36

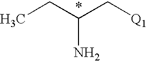

37

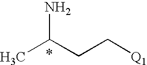

38

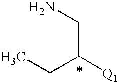

39

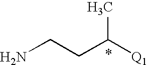

40

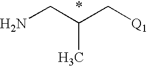

41

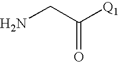

42

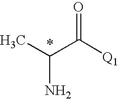

43

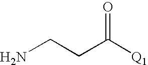

44

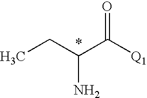

45

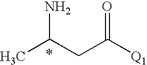

46

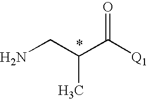

47

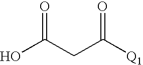

48

TABLE 2c

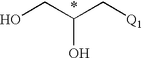

49

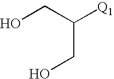

50

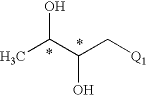

51

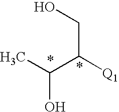

52

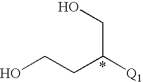

53

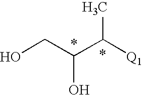

54

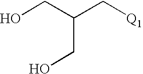

55

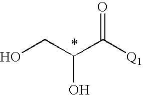

56

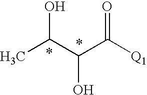

57

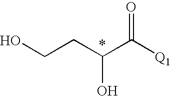

58

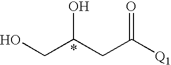

59

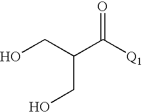

60

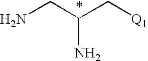

61

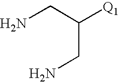

62

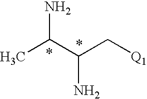

63

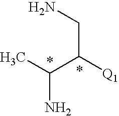

64

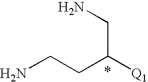

65

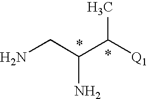

66

TABLE 2d

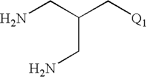

67

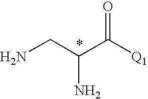

68

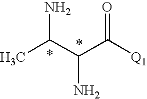

69

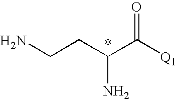

70

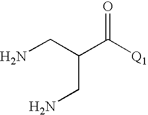

71

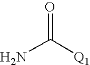

72

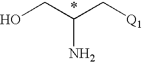

73

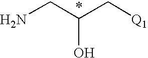

74

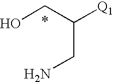

75

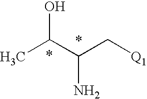

76

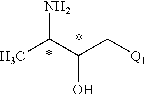

77

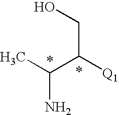

78

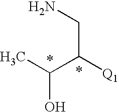

79

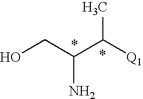

80

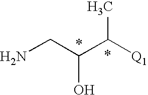

81

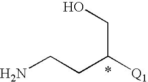

82

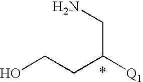

83

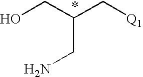

84

TABLE 2e

| | |
|---|---|
| 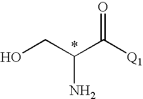 | 85 |
| 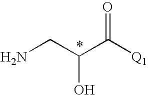 | 86 |
| 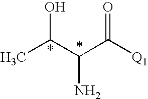 | 87 |
| 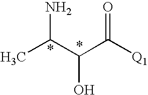 | 88 |
| 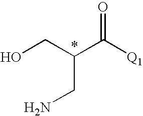 | 89 |
| 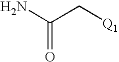 | 90 |
| 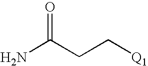 | 91 |
| 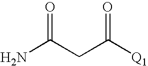 | 92 |
| 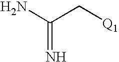 | 93 |
| 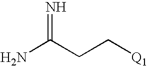 | 94 |
| 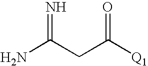 | 95 |
| 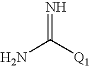 | 96 |
| 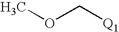 | 97 |
| 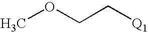 | 98 |
| 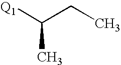 | 100 |
| 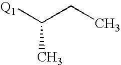 | 101 |
| 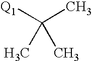 | 102 |

TABLE 2f

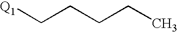

103

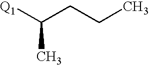

104

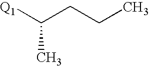

105

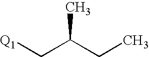

106

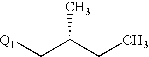

107

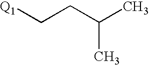

108

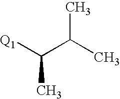

109

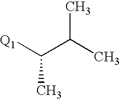

110

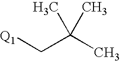

111

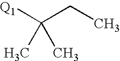

112

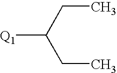

113

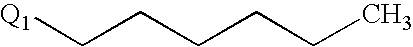

114

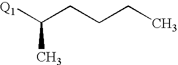

115

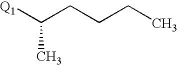

116

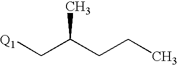

117

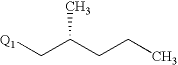

118

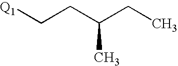

119

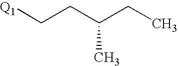

120

TABLE 2g

| 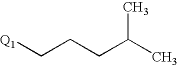 | 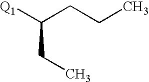 |
|---|---|
| 121 | 122 |
| 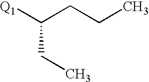 | 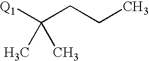 |
| 123 | 124 |
| 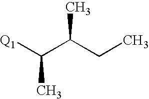 | 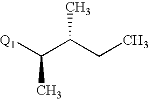 |
| 125 | 126 |
| 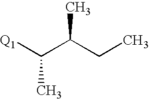 | 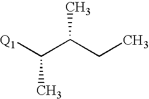 |
| 127 | 128 |
| 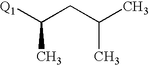 | 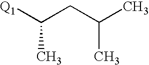 |
| 129 | 130 |
| 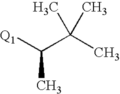 | 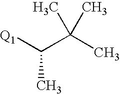 |
| 131 | 132 |
| 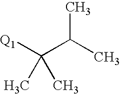 | 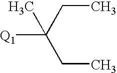 |
| 133 | 134 |
| 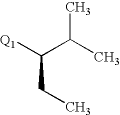 | 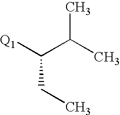 |
| 135 | 136 |
| 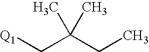 | 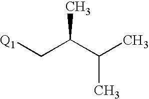 |
| 137 | 138 |

TABLE 2h

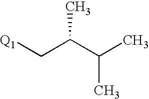

139

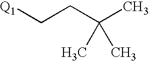

140

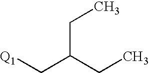

141

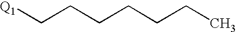

142

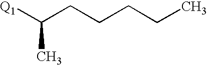

143

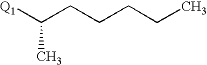

144

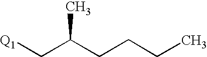

145

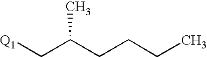

146

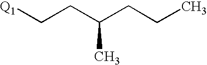

147

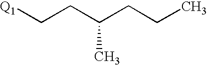

148

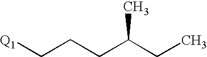

149

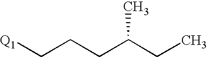

150

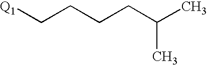

151

TABLE 2i

| 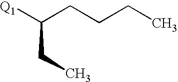 | 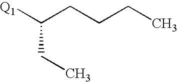 |
|---|---|
| 152 | 153 |
| 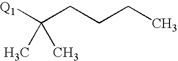 | 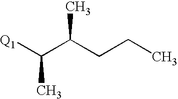 |
| 154 | 155 |
| 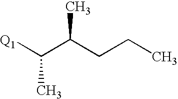 | 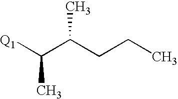 |
| 156 | 157 |
| 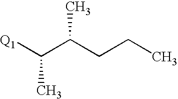 | 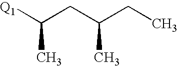 |
| 158 | 159 |
| 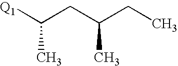 | 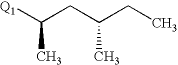 |
| 160 | 161 |
| 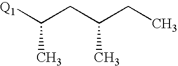 | 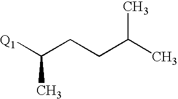 |
| 162 | 163 |
| 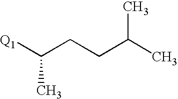 | 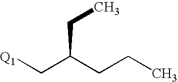 |
| 164 | 165 |
| 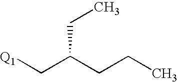 | 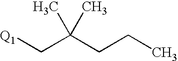 |
| 166 | 167 |
| 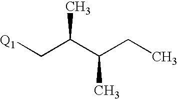 | 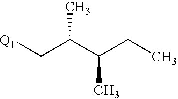 |
| 168 | 169 |

TABLE 2j

| 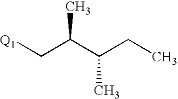 | 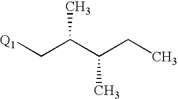 |
|---|---|
| 170 | 171 |
| 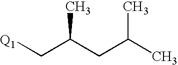 | 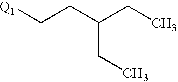 |
| 172 | 173 |
| 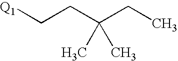 | 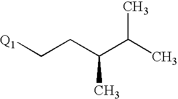 |
| 174 | 175 |
| 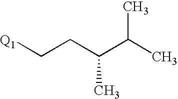 | 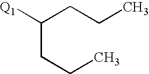 |
| 176 | 177 |
| 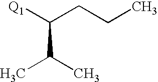 | 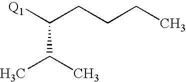 |
| 178 | 179 |
| 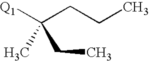 | 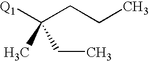 |
| 180 | 181 |
| 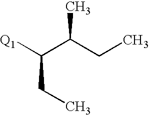 | 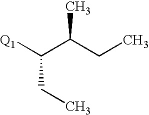 |
| 182 | 183 |
| 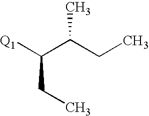 | 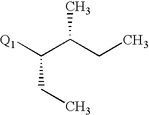 |
| 184 | 185 |
| 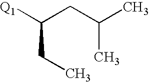 | 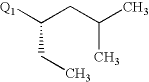 |
| 186 | 187 |

TABLE 2k

| 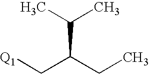 | 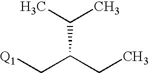 |
|---|---|
| 188 | 189 |
| 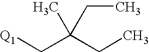 | 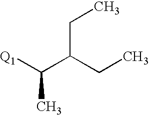 |
| 190 | 191 |
| 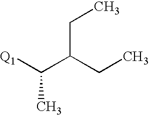 | 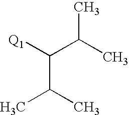 |
| 192 | 193 |
| 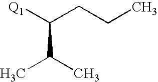 | 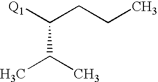 |
| 194 | 195 |
| 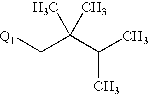 | 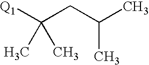 |
| 196 | 197 |
| 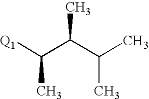 | 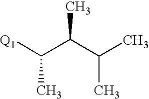 |
| 198 | 199 |
| 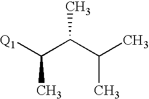 | 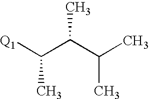 |
| 200 | 201 |
| 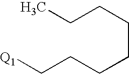 | 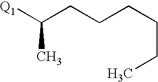 |
| 202 | 203 |
| 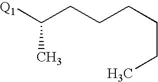 | |
| 204 | |

TABLE 2l

| 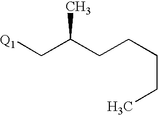 | 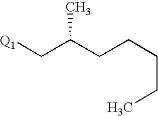 |
|---|---|
| 205 | 206 |
| 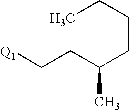 | 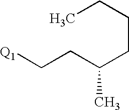 |
| 207 | 208 |
| 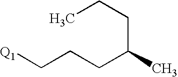 | 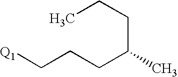 |
| 209 | 210 |
| 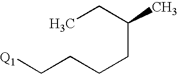 | 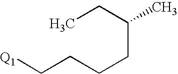 |
| 211 | 212 |
| 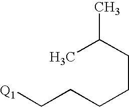 | 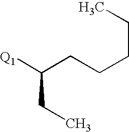 |
| 213 | 214 |
| 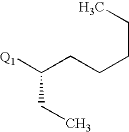 | 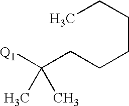 |
| 215 | 216 |
| 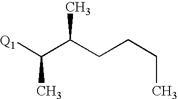 | 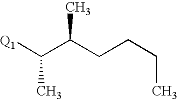 |
| 217 | 218 |
| 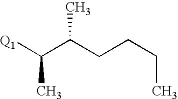 | 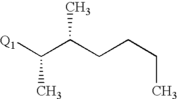 |
| 219 | 220 |
| 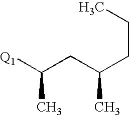 | 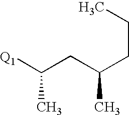 |
| 221 | 222 |

TABLE 2m

| 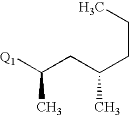 | 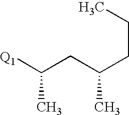 |
|---|---|
| 223 | 224 |
| 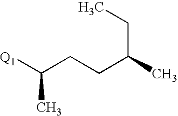 | 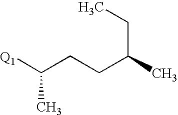 |
| 225 | 226 |
| 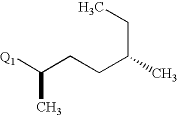 | 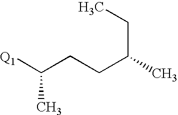 |
| 227 | 228 |
| 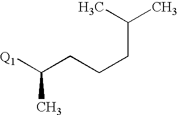 | 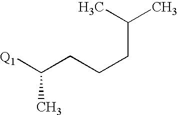 |
| 229 | 230 |
| 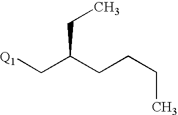 | 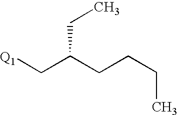 |
| 231 | 232 |
| 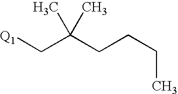 | 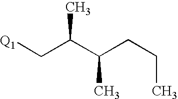 |
| 233 | 234 |
| 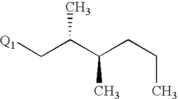 | 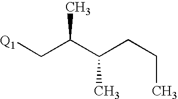 |
| 235 | 236 |
| 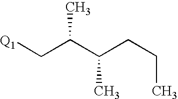 | 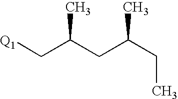 |
| 237 | 238 |
| 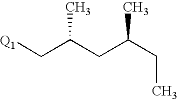 | 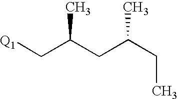 |
| 239 | 240 |

TABLE 2n

| 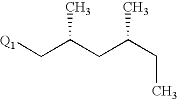 | 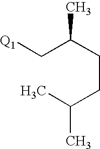 |
|---|---|
| 241 | 242 |
| 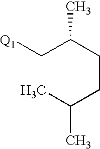 | 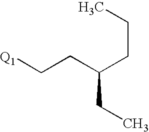 |
| 243 | 244 |
| 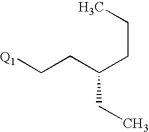 | 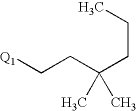 |
| 245 | 246 |
| 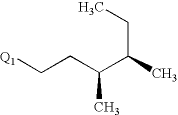 | 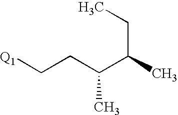 |
| 247 | 248 |
| 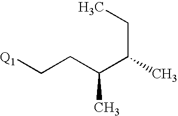 | 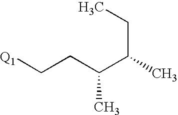 |
| 249 | 250 |
| 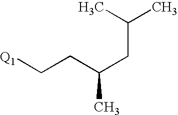 | 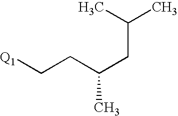 |
| 251 | 252 |
| 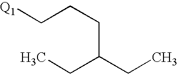 | 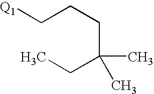 |
| 253 | 254 |
| 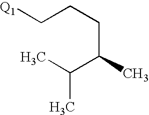 | 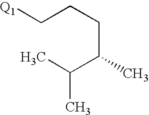 |
| 255 | 256 |
| 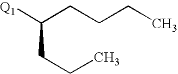 | 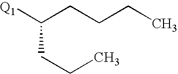 |
| 257 | 258 |

TABLE 2o

| 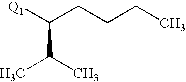 | 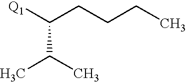 |
|---|---|
| 259 | 260 |
| 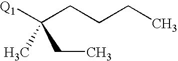 | 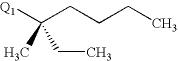 |
| 261 | 262 |
| 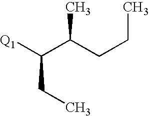 | 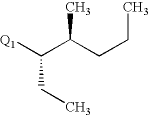 |
| 263 | 264 |
| 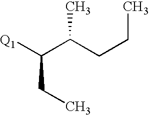 | 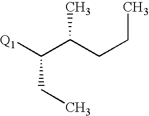 |
| 265 | 266 |
| 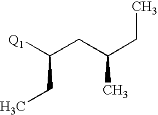 | 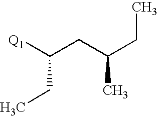 |
| 267 | 268 |
| 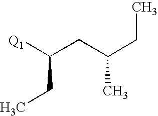 | 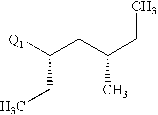 |
| 269 | 270 |
| 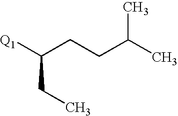 | 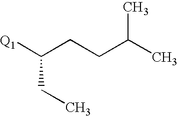 |
| 271 | 272 |
| 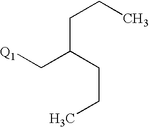 | 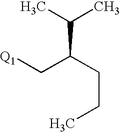 |
| 273 | 274 |
| 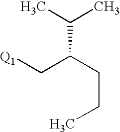 | 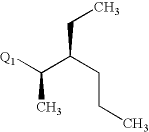 |
| 275 | 276 |

TABLE 2p

| 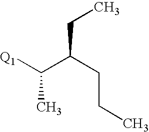 | 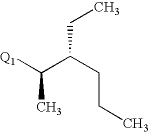 |
|---|---|
| 277 | 278 |
| 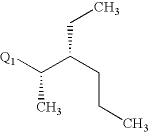 | 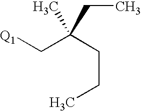 |
| 279 | 280 |
| 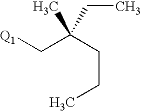 | 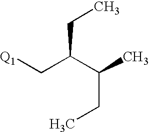 |
| 281 | 282 |
| 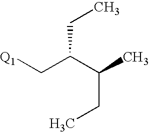 | 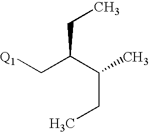 |
| 283 | 284 |
| 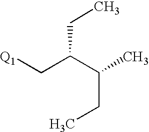 | 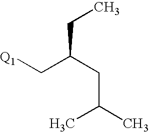 |
| 285 | 286 |
| 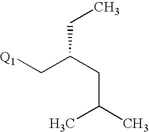 | 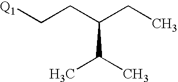 |
| 287 | 288 |
| 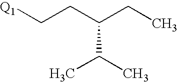 | 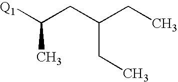 |
| 289 | 290 |
| 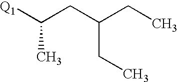 | 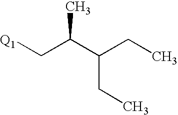 |
| 291 | 292 |
| 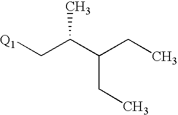 | 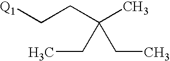 |
| 293 | 294 |

TABLE 2q

| 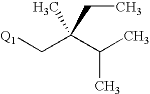 | 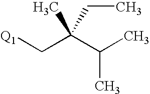 |
|---|---|
| 295 | 296 |
| 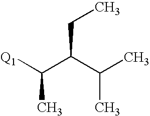 | 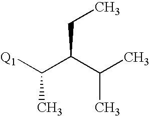 |
| 297 | 298 |
| 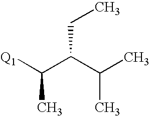 | 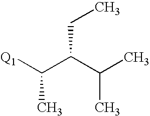 |
| 299 | 300 |
| 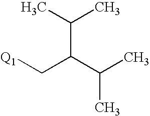 | 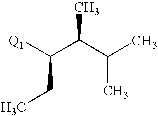 |
| 301 | 302 |
| 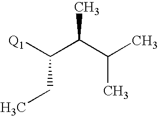 | 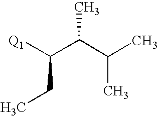 |
| 303 | 304 |
| 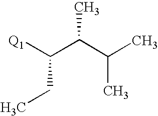 | 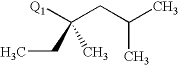 |
| 305 | 306 |
| 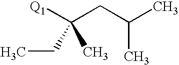 | 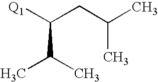 |
| 307 | 308 |
| 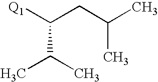 | 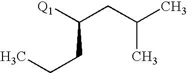 |
| 309 | 310 |
| 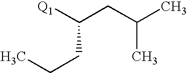 | 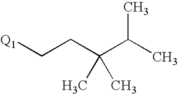 |
| 311 | 312 |

TABLE 2r

| 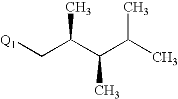 | 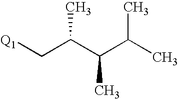 |
|---|---|
| 313 | 314 |
| 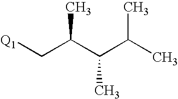 | 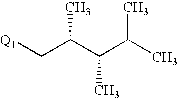 |
| 315 | 316 |
| 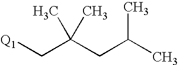 | 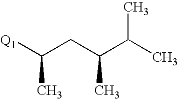 |
| 317 | 318 |
| 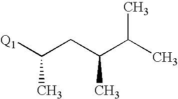 | 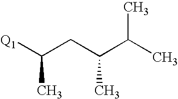 |
| 319 | 320 |
| 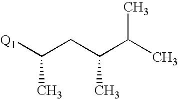 | 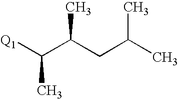 |
| 321 | 322 |
| 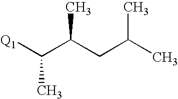 | 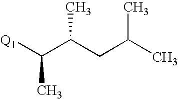 |
| 323 | 324 |
| 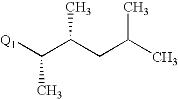 | 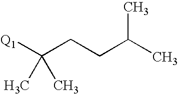 |
| 325 | 326 |
| 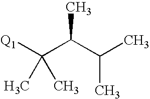 | 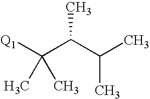 |
| 327 | 328 |
| 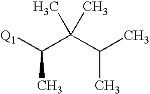 | 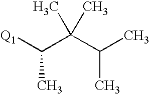 |
| 329 | 330 |

TABLE 2s

| 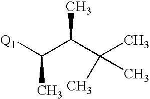 | 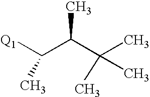 |
|---|---|
| 331 | 332 |
| 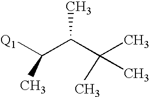 | 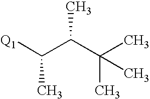 |
| 333 | 334 |
| 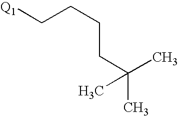 | 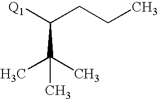 |
| 335 | 336 |
| 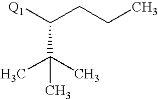 | 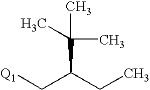 |
| 337 | 338 |
| 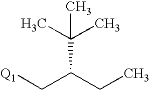 | 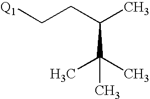 |
| 339 | 340 |
| 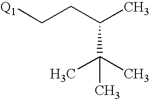 | 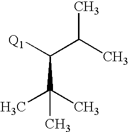 |
| 341 | 342 |
| 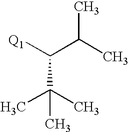 | 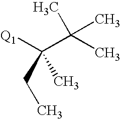 |
| 343 | 344 |
| 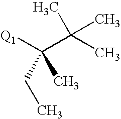 | 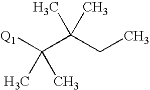 |
| 345 | 346 |
| 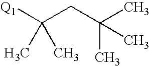 | 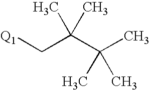 |
| 347 | 348 |

TABLE 2t

| 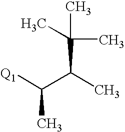 | 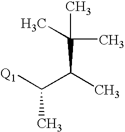 |
|---|---|
| 349 | 350 |
| 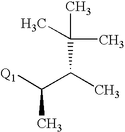 | 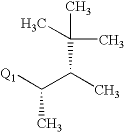 |
| 351 | 352 |
| 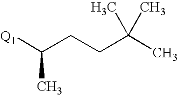 | 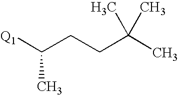 |
| 353 | 354 |
| 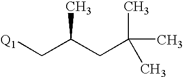 | 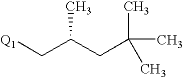 |
| 355 | 356 |
| 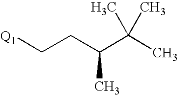 | 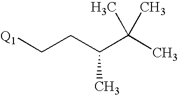 |
| 357 | 358 |
| 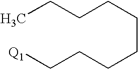 | 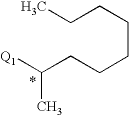 |
| 359 | 360 |
| 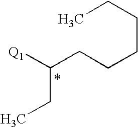 | 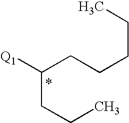 |
| 361 | 362 |
| 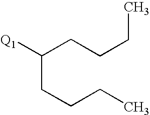 | 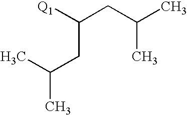 |
| 363 | 364 |
| 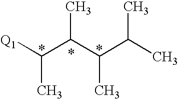 | 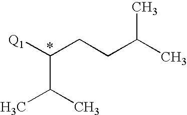 |
| 365 | 366 |

TABLE 2u

| 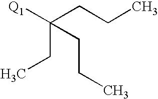 | 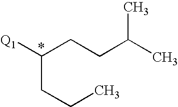 |
|---|---|
| 367 | 368 |
| 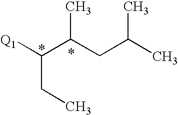 | 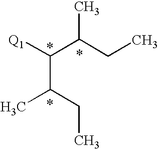 |
| 369 | 370 |
| 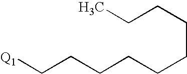 | 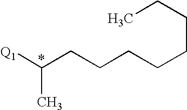 |
| 371 | 372 |
| 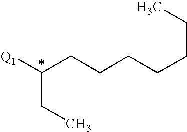 | 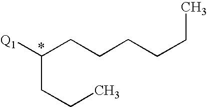 |
| 373 | 374 |
| 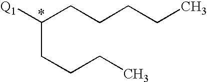 | 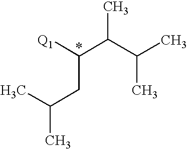 |
| 375 | 376 |
| 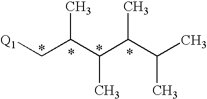 | 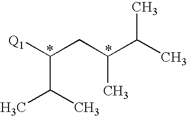 |
| 377 | 378 |
| 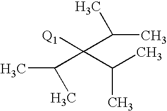 | 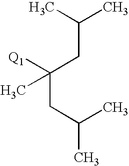 |
| 379 | 380 |
| 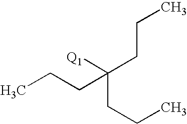 | 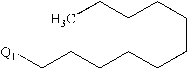 |
| 381 | 382 |
| 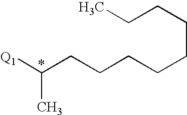 | 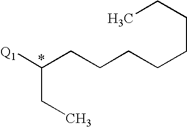 |
| 383 | 384 |

TABLE 2v

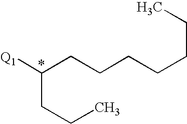

385

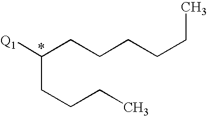

386

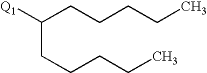

387

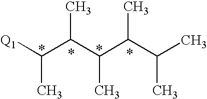

388

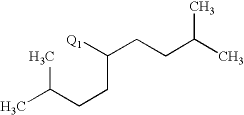

389

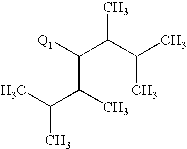

390

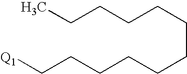

391

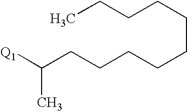

392

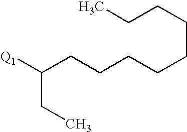

393

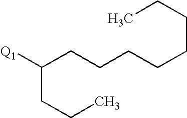

394

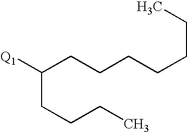

395

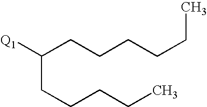

396

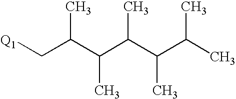

397

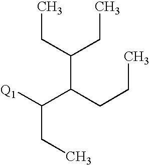

398

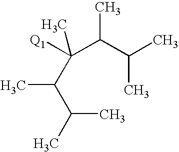

399

TABLE 2w

| 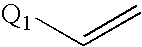 | 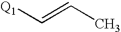 |
|---|---|
| 400 | 401 |
| 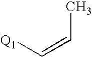 | 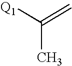 |
| 402 | 403 |
| 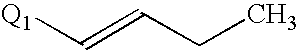 | 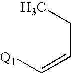 |
| 404 | 405 |
| 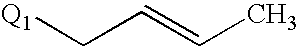 | 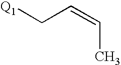 |
| 406 | 407 |
| 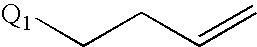 | 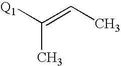 |
| 408 | 409 |
| 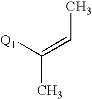 | 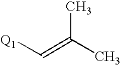 |
| 410 | 411 |
| 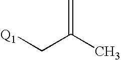 | 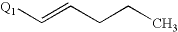 |
| 412 | 413 |
| 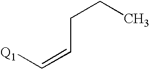 | 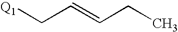 |
| 414 | 415 |
| 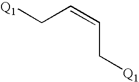 | 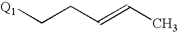 |
| 416 | 417 |
| 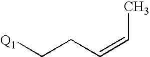 | 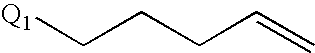 |
| 418 | 419 |

TABLE 2x

| 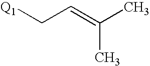 | 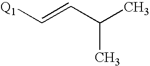 |
|---|---|
| 420 | 421 |
| 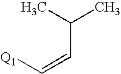 | 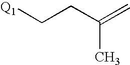 |
| 422 | 423 |
| 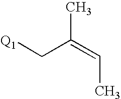 | 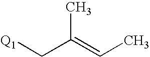 |
| 424 | 425 |
| 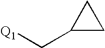 | 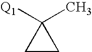 |
| 426 | 427 |
| 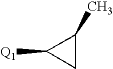 | 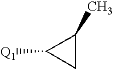 |
| 428 | 429 |
| 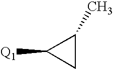 | 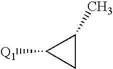 |
| 430 | 431 |
| 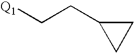 | 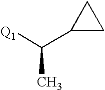 |
| 432 | 433 |
| 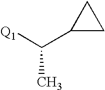 | 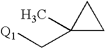 |
| 434 | 435 |
| 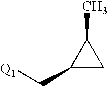 | 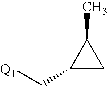 |
| 436 | 437 |
| 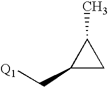 | 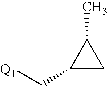 |
| 438 | 439 |

TABLE 2y

| 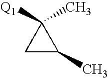 | 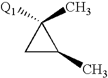 |
|---|---|
| 440 | 441 |
| 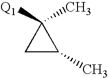 | 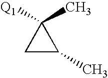 |
| 442 | 443 |
| 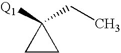 | 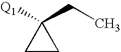 |
| 444 | 445 |
| 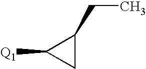 | 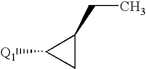 |
| 446 | 447 |
| 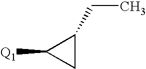 | 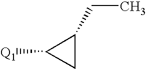 |
| 448 | 449 |
| 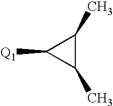 | 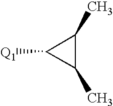 |
| 450 | 451 |
| 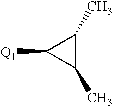 | 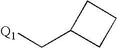 |
| 452 | 453 |
| 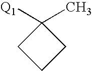 | 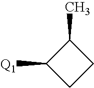 |
| 454 | 455 |
| 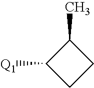 | 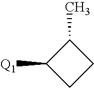 |
| 456 | 457 |
| 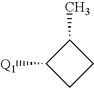 | 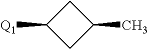 |
| 458 | 459 |
| 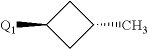 | 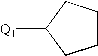 |
| 460 | 461 |
| 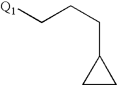 | 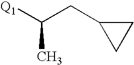 |
| 462 | 666 |

TABLE 2z

| 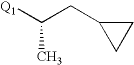 | 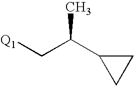 |
|---|---|
| 463 | 464 |
| 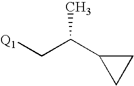 | 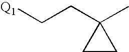 |
| 465 | 466 |
| 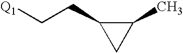 | 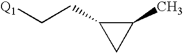 |
| 467 | 468 |
| 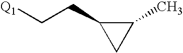 | 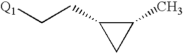 |
| 469 | 470 |
| 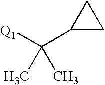 | 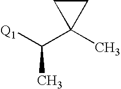 |
| 471 | 472 |
| 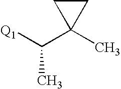 | 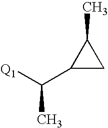 |
| 473 | 474 |
| 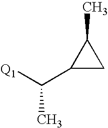 | 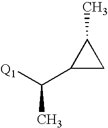 |
| 475 | 476 |
| 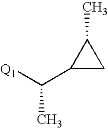 | 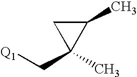 |
| 477 | 478 |
| 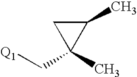 | 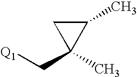 |
| 479 | 480 |
| 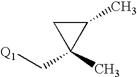 | 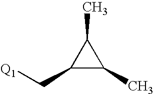 |
| 481 | 482 |
| 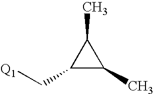 | |
| 483 | |

TABLE 2aa

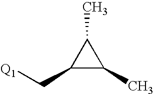

484

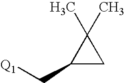

485

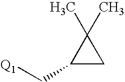

486

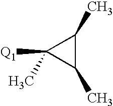

487

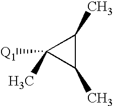

488

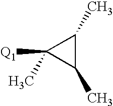

489

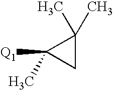

490

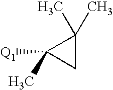

491

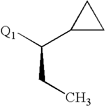

492

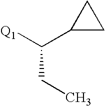

493

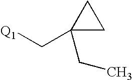

494

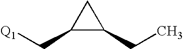

495

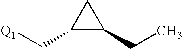

496

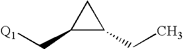

497

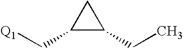

498

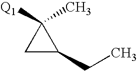

499

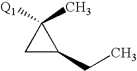

500

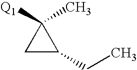

501

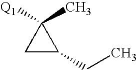

502

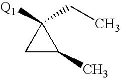

503

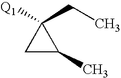

504

TABLE 2ab

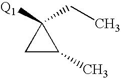

505

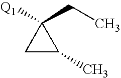

506

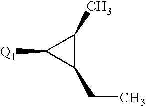

507

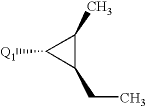

508

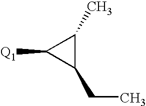

509

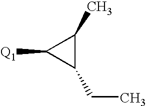

510

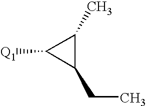

511

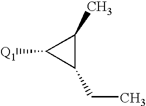

512

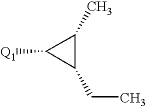

513

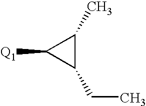

514

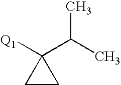

515

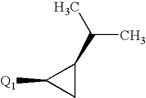

516

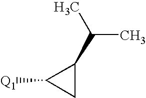

517

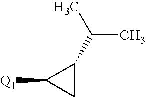

518

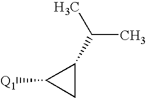

519

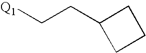

520

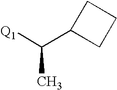

521

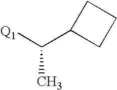

522

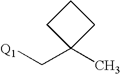

523

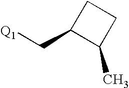

524

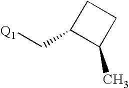

525

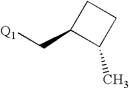

526

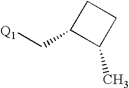

527

TABLE 2ac

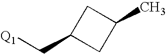

528

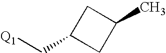

529

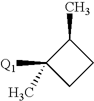

530

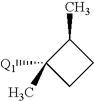

531

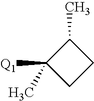

532

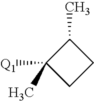

533

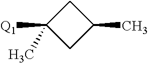

534

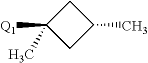

535

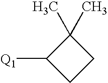

536

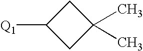

537

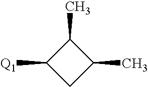

538

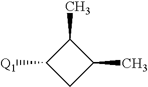

539

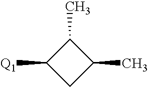

540

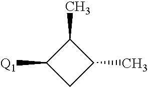

541

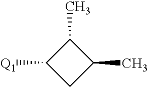

542

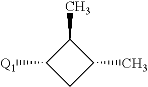

543

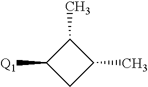

544

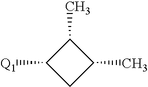

545

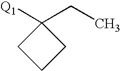

546

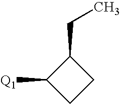

547

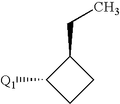

548

TABLE 2ad

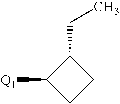

549

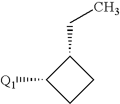

550

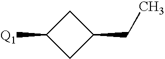

551

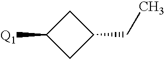

552

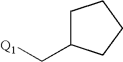

553

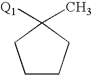

554

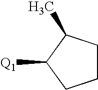

555

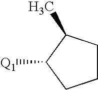

556

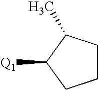

557

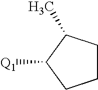

558

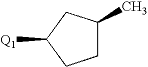

559

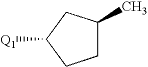

560

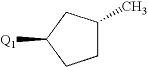

561

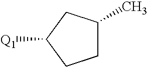

562

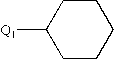

563

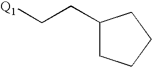

564

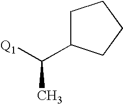

565

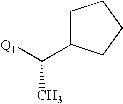

566

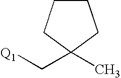

567

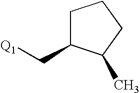

568

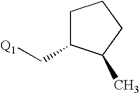

569

TABLE 2ae

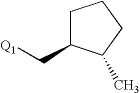

570

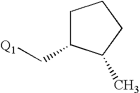

571

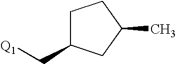

572

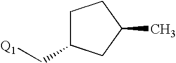

573

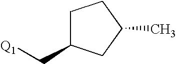

574

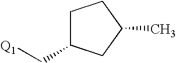

575

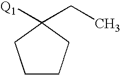

576

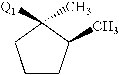

577

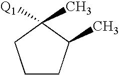

578

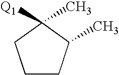

579

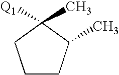

580

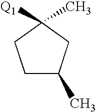

581

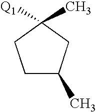

582

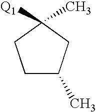

583

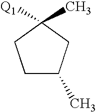

584

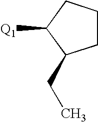

585

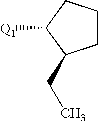

586

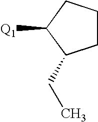

587

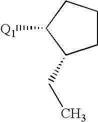

588

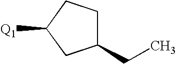

589

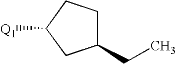

590

TABLE 2af

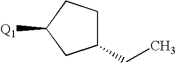

591

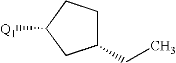

592

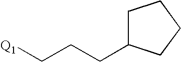

593

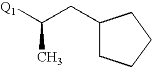

594

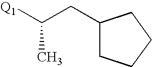

595

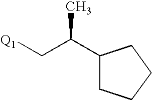

596

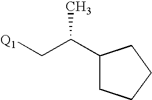

597

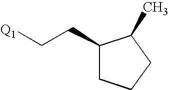

598

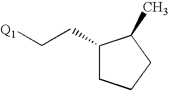

599

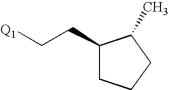

600

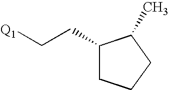

601

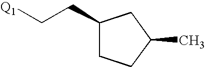

602

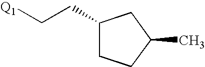

603

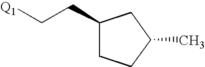

604

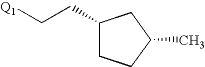

605

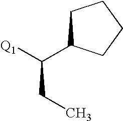

606

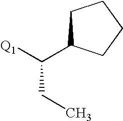

607

TABLE 2ag

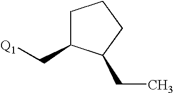

608

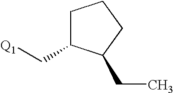

609

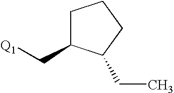

610

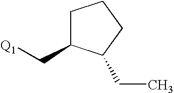

611

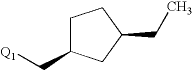

612

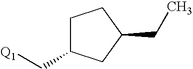

613

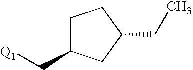

614

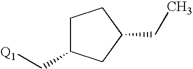

615

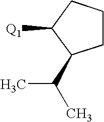

616

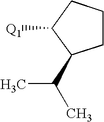

617

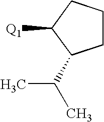

618

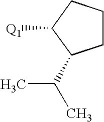

619

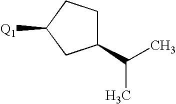

620

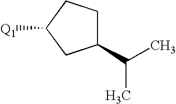

621

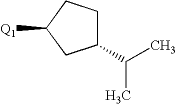

622

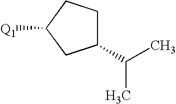

623

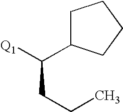

624

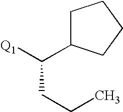

625

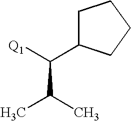

626

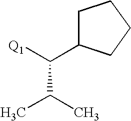

627

TABLE 2ah

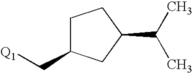

628

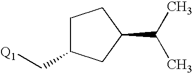

629

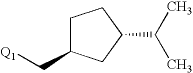

630

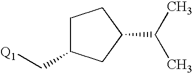

631

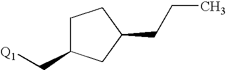

632

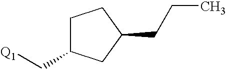

633

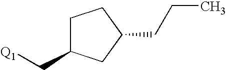

634

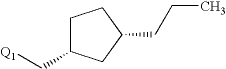

635

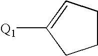

636

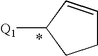

637

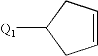

638

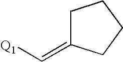

639

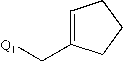

640

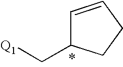

641

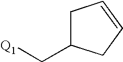

642

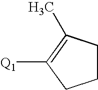

643

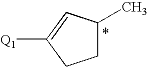

644

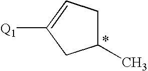

645

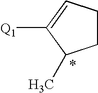

646

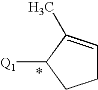

647

TABLE 2ai

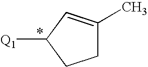

648

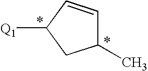

649

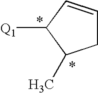

650

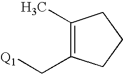

651

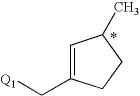

652

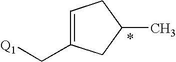

653

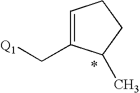

654

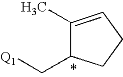

655

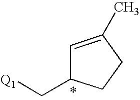

656

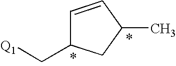

657

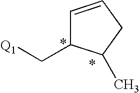

658

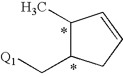

659

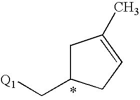

660

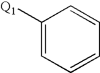

661

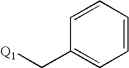

662

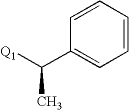

663

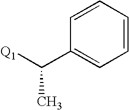

664

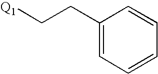

665

TABLE 3a

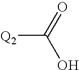

a

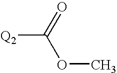

b

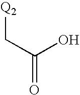

c

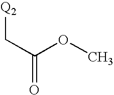

d

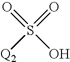

e

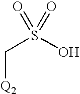

f

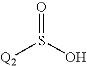

g

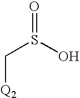

h

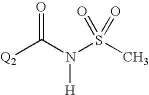

i

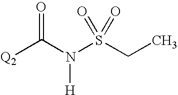

j

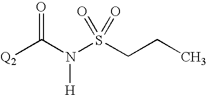

k

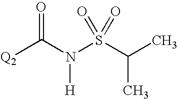

l

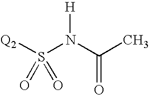

m

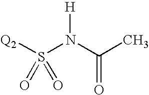

n

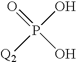

o

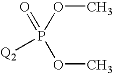

p

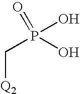

q

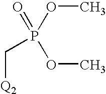

r

TABLE 3b

| | |
|---|---|
| 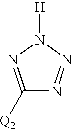 | s |
| 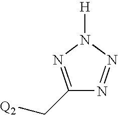 | t |
| 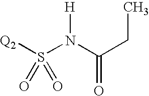 | u |
| 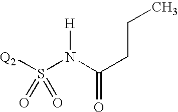 | v |
| 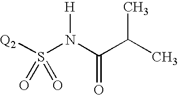 | w |
| 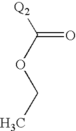 | x |
| 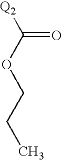 | y |
| 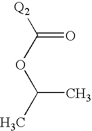 | z |
| 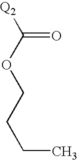 | A |
| 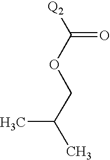 | B |
| 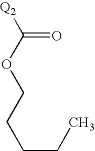 | C |
| 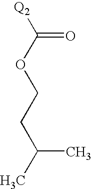 | D |
| 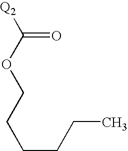 | E |
| 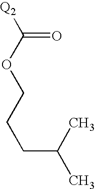 | F |

TABLE 4a

| | |
|---|---|
| Q₃—OH | 1 |
| Q₃—N₃ | 2 |
| Q₃—NO₂ | 3 |
| Q₃—NH₂ | 4 |
| 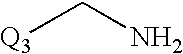 | 5 |
| 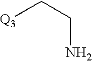 | 6 |
| 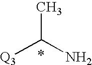 | 7 |
| 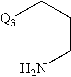 | 8 |
| 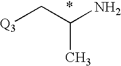 | 9 |
| 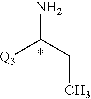 | 10 |
| 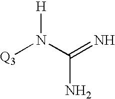 | 11 |
| 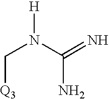 | 12 |
| 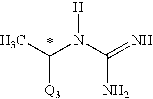 | 13 |
| 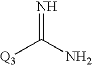 | 14 |
| 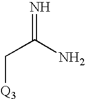 | 15 |
| 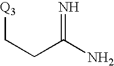 | 16 |
| 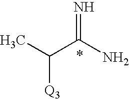 | 17 |
| 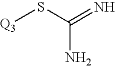 | 18 |
| 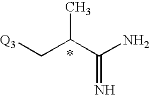 | 19 |
| 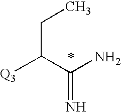 | 20 |
| 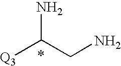 | 21 |
| 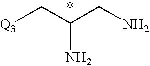 | 22 |
| 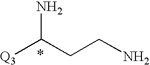 | 23 |
| 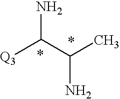 | 24 |

TABLE 4b

| | |
|---|---|
| 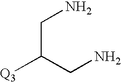 | 25 |
| 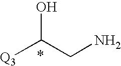 | 26 |
| 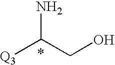 | 27 |
| 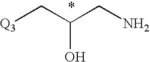 | 28 |
| 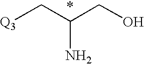 | 29 |
| 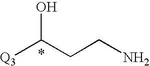 | 30 |
| 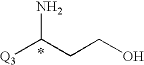 | 31 |
| 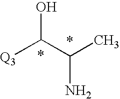 | 32 |
| 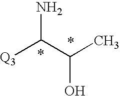 | 33 |
| 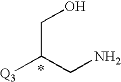 | 34 |
| Q₃—CN | 35 |
| 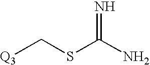 | 36 |
| 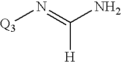 | 37 |
| 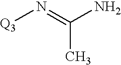 | 38 |
| 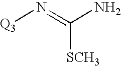 | 39 |
| 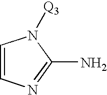 | 40 |
| 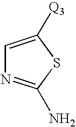 | 41 |
| 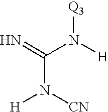 | 42 |
| 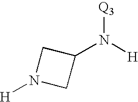 | 43 |
| 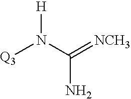 | 44 |
| 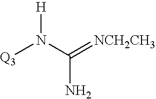 | 45 |

TABLE 4c

| | |
|---|---|
| 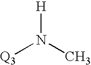 | 46 |
| 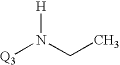 | 47 |
| 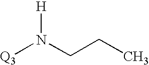 | 48 |
| 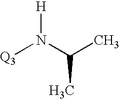 | 49 |
| 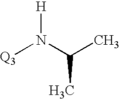 | 50 |
| 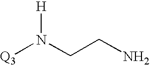 | 51 |
| 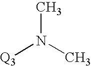 | 52 |
| 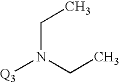 | 53 |

TABLE 5a

| | |
|---|---|
| H—Q₄ | a |
| H₃C—Q₄ | b |
| 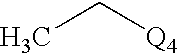 | c |
| 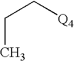 | d |
| 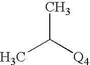 | e |
| 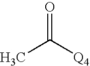 | f |
| 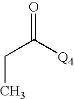 | g |
| 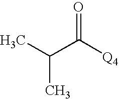 | h |
| 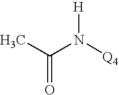 | i |
| 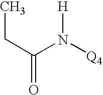 | j |
| 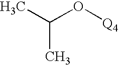 | k |
| 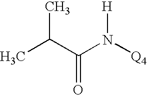 | l |
| 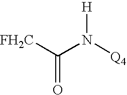 | m |
| 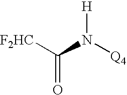 | n |
| 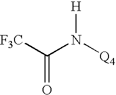 | o |
| 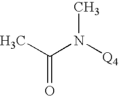 | p |
| 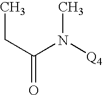 | q |
| 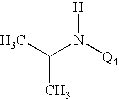 | r |
| 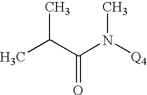 | s |
| 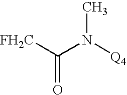 | t |
| 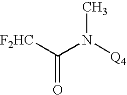 | u |

TABLE 5b

| | |
|---|---|
| 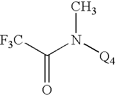 | v |
| 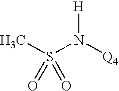 | w |
| 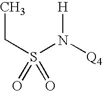 | x |
| 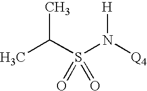 | y |
| 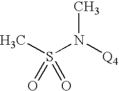 | z |
| 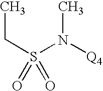 | aa |
| 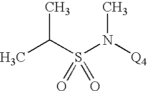 | ab |
| 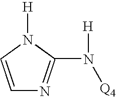 | ac |
| 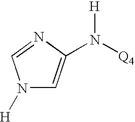 | ad |
| 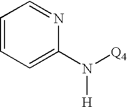 | ae |
| 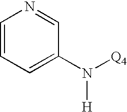 | af |
| 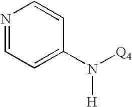 | ag |
| 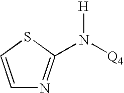 | ah |
| 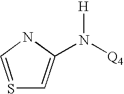 | ai |
| 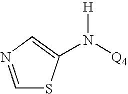 | aj |
| 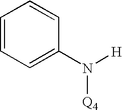 | ak |
| 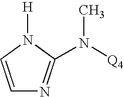 | al |
| 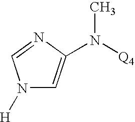 | am |
| 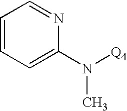 | an |
| 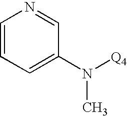 | ao |

TABLE 5c

| | |
|---|---|
| 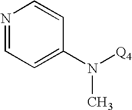 | ap |
| 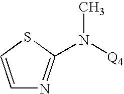 | aq |
| 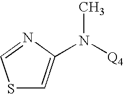 | ar |
| 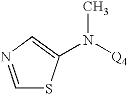 | as |
| H₂N—Q₄ | at |
| 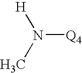 | au |
| 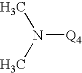 | av |
| 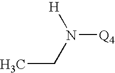 | aw |
| 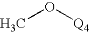 | ax |
| 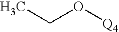 | ay |
| 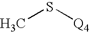 | az |
| 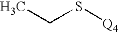 | ba |
| 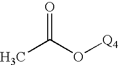 | bb |
| 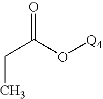 | bc |
| 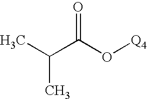 | bd |
| 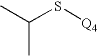 | be |
| 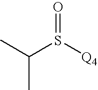 | bf |
| 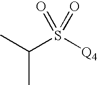 | bg |
| 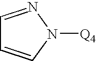 | bh |
| 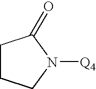 | bi |
| 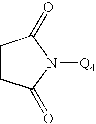 | bj |
| 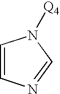 | bk |

TABLE 6
Exemplary Enumerated Compounds

A.17.a.4.i; A.17.a.4.v; A.17.a.6.i; A.17.a.6.v; A.17.a.11.i; A.17.a.11.v; A.17.a.14.i;
A.17.a.14.v; A.17.a.15.i; A.17.a.15.v; A.17.a.18.i; A.17.a.18.v; A.17.a.25.i;
A.17.a.25.v; A.17.e.4.i; A.17.e.4.v; A.17.e.6.i; A.17.e.6.v; A.17.e.11.i; A.17.e.11.v;
A.17.e.14.i; A.17.e.14.v; A.17.e.15.i; A.17.e.15.v; A.17.e.18.i; A.17.e.18.v;
A.17.e.25.i; A.17.e.25.v; A.17.g.4.i; A.17.g.4.v; A.17.g.6.i; A.17.g.6.v; A.17.g.11.i;
A.17.g.11.v; A.17.g.14.i; A.17.g.14.v; A.17.g.15.i; A.17.g.15.v; A.17.g.18.i;
A.17.g.18.v; A.17.g.25.i; A.17.g.25.v; A.17.l.4.i; A.17.l.4.v; A.17.l.6.i; A.17.l.6.v;
A.17.l.11.i; A.17.l.11.v; A.17.l.14.i; A.17.l.14.v; A.17.l.15.i; A.17.l.15.v; A.17.l.18.i;
A.17.l.18.v; A.17.l.25.i; A.17.l.25.v; A.17.m.4.i; A.17.m.4.v; A.17.m.6.i;
A.17.m.6.v; A.17.m.11.i; A.17.m.11.v; A.17.m.14.i; A.17.m.14.v; A.17.m.15.i;
A.17.m.15.v; A.17.m.18.i; A.17.m.18.v; A.17.m.25.i; A.17.m.25.v; A.17.o.4.i;
A.17.o.4.v; A.17.o.6.i; A.17.o.6.v; A.17.o.11.i; A.17.o.11.v; A.17.o.14.i;
A.17.o.14.v; A.17.o.15.i; A.17.o.15.v; A.17.o.18.i; A.17.o.18.v; A.17.o.25.i;
A.17.o.25.v; A.33.a.4.i; A.33.a.4.v; A.33.a.6.i; A.33.a.6.v; A.33.a.11.i; A.33.a.11.v;
A.33.a.14.i; A.33.a.14.v; A.33.a.15.i; A.33.a.15.v; A.33.a.18.i; A.33.a.18.v;
A.33.a.25.i; A.33.a.25.v; A.33.e.4.i; A.33.e.4.v; A.33.e.6.i; A.33.e.6.v; A.33.e.11.i;
A.33.e.11.v; A.33.e.14.i; A.33.e.14.v; A.33.e.15.i; A.33.e.15.v; A.33.e.18.i;
A.33.e.18.v; A.33.e.25.i; A.33.e.25.v; A.33.g.4.i; A.33.g.4.v; A.33.g.6.i; A.33.g.6.v;
A.33.g.11.i; A.33.g.11.v; A.33.g.14.i; A.33.g.14.v; A.33.g.15.i; A.33.g.15.v;
A.33.g.18.i; A.33.g.18.v; A.33.g.25.i; A.33.g.25.v; A.33.l.4.i; A.33.l.4.v; A.33.l.6.i;
A.33.l.6.v; A.33.l.11.i; A.33.l.11.v; A.33.l.14.i; A.33.l.14.v; A.33.l.15.i; A.33.l.15.v;
A.33.l.18.i; A.33.l.18.v; A.33.l.25.i; A.33.l.25.v; A.33.m.4.i; A.33.m.4.v; A.33.m.6.i;
A.33.m.6.v; A.33.m.11.i; A.33.m.11.v; A.33.m.14.i; A.33.m.14.v; A.33.m.15.i;
A.33.m.15.v; A.33.m.18.i; A.33.m.18.v; A.33.m.25.i; A.33.m.25.v; A.33.o.4.i;
A.33.o.4.v; A.33.o.6.i; A.33.o.6.v; A.33.o.11.i; A.33.o.11.v; A.33.o.14.i;
A.33.o.14.v; A.33.o.15.i; A.33.o.15.v; A.33.o.18.i; A.33.o.18.v; A.33.o.25.i;
A.33.o.25.v; A.49.a.4.i; A.49.a.4.v; A.49.a.6.i; A.49.a.6.v; A.49.a.11.i; A.49.a.11.v;
A.49.a.14.i; A.49.a.14.v; A.49.a.15.i; A.49.a.15.v; A.49.a.18.i; A.49.a.18.v;
A.49.a.25.i; A.49.a.25.v; A.49.e.4.i; A.49.e.4.v; A.49.e.6.i; A.49.e.6.v; A.49.e.11.i;
A.49.e.11.v; A.49.e.14.i; A.49.e.14.v; A.49.e.15.i; A.49.e.15.v; A.49.e.18.i;
A.49.e.18.v; A.49.e.25.i; A.49.e.25.v; A.49.g.4.i; A.49.g.4.v; A.49.g.6.i; A.49.g.6.v;
A.49.g.11.i; A.49.g.11.v; A.49.g.14.i; A.49.g.14.v; A.49.g.15.i; A.49.g.15.v;
A.49.g.18.i; A.49.g.18.v; A.49.g.25.i; A.49.g.25.v; A.49.l.4.i; A.49.l.4.v; A.49.l.6.i;
A.49.l.6.v; A.49.l.11.i; A.49.l.11.v; A.49.l.14.i; A.49.l.14.v; A.49.l.15.i; A.49.l.15.v;
A.49.l.18.i; A.49.l.18.v; A.49.l.25.i; A.49.l.25.v; A.49.m.4.i; A.49.m.4.v; A.49.m.6.i;
A.49.m.6.v; A.49.m.11.i; A.49.m.11.v; A.49.m.14.i; A.49.m.14.v; A.49.m.15.i;
A.49.m.15.v; A.49.m.18.i; A.49.m.18.v; A.49.m.25.i; A.49.m.25.v; A.49.o.4.i;
A.49.o.4.v; A.49.o.6.i; A.49.o.6.v; A.49.o.11.i; A.49.o.11.v; A.49.o.14.i;
A.49.o.14.v; A.49.o.15.i; A.49.o.15.v; A.49.o.18.i; A.49.o.18.v; A.49.o.25.i;
A.49.o.25.v; B.17.a.4.i; B.17.a.4.v; B.17.a.6.i; B.17.a.6.v; B.17.a.11.i; B.17.a.11.v;
B.17.a.14.i; B.17.a.14.v; B.17.a.15.i; B.17.a.15.v; B.17.a.18.i; B.17.a.18.v; B.17.a.25.i;
B.17.a.25.v; B.17.e.4.i; B.17.e.4.v; B.17.e.6.i; B.17.e.6.v; B.17.e.11.i; B.17.e.11.v;
B.17.e.14.i; B.17.e.14.v; B.17.e.15.i; B.17.e.15.v; B.17.e.18.i; B.17.e.18.v; B.17.e.25.i;
B.17.e.25.v; B.17.g.4.i; B.17.g.4.v; B.17.g.6.i; B.17.g.6.v; B.17.g.11.i; B.17.g.11.v;
B.17.g.14.i; B.17.g.14.v; B.17.g.15.i; B.17.g.15.v; B.17.g.18.i; B.17.g.18.v;
B.17.g.25.i; B.17.g.25.v; B.17.l.4.i; B.17.l.4.v; B.17.l.6.i; B.17.l.6.v; B.17.l.11.i;
B.17.l.11.v; B.17.l.14.i; B.17.l.14.v; B.17.l.15.i; B.17.l.15.v; B.17.l.18.i; B.17.l.18.v;
B.17.l.25.i; B.17.l.25.v; B.17.m.4.i; B.17.m.4.v; B.17.m.6.i; B.17.m.6.v; B.17.m.11.i;
B.17.m.11.v; B.17.m.14.i; B.17.m.14.v; B.17.m.15.i; B.17.m.15.v; B.17.m.18.i;
B.17.m.18.v; B.17.m.25.i; B.17.m.25.v; B.17.o.4.i; B.17.o.4.v; B.17.o.6.i; B.17.o.6.v;
B.17.o.11.i; B.17.o.11.v; B.17.o.14.i; B.17.o.14.v; B.17.o.15.i; B.17.o.15.v;
B.17.o.18.i; B.17.o.18.v; B.17.o.25.i; B.17.o.25.v; B.33.a.4.i; B.33.a.4.v; B.33.a.6.i;
B.33.a.6.v; B.33.a.11.i; B.33.a.11.v; B.33.a.14.i; B.33.a.14.v; B.33.a.15.i; B.33.a.15.v;
B.33.a.18.i; B.33.a.18.v; B.33.a.25.i; B.33.a.25.v; B.33.e.4.i; B.33.e.4.v; B.33.e.6.i;
B.33.e.6.v; B.33.e.11.i; B.33.e.11.v; B.33.e.14.i; B.33.e.14.v; B.33.e.15.i; B.33.e.15.v;
B.33.e.18.i; B.33.e.18.v; B.33.e.25.i; B.33.e.25.v; B.33.g.4.i; B.33.g.4.v; B.33.g.6.i;
B.33.g.6.v; B.33.g.11.i; B.33.g.11.v; B.33.g.14.i; B.33.g.14.v; B.33.g.15.i;
B.33.g.15.v; B.33.g.18.i; B.33.g.18.v; B.33.g.25.i; B.33.g.25.v; B.33.l.4.i; B.33.l.4.v;
B.33.l.6.i; B.33.l.6.v; B.33.l.11.i; B.33.l.11.v; B.33.l.14.i; B.33.l.14.v; B.33.l.15.i;
B.33.l.15.v; B.33.l.18.i; B.33.l.18.v; B.33.l.25.i; B.33.l.25.v; B.33.m.4.i; B.33.m.4.v;
B.33.m.6.i; B.33.m.6.v; B.33.m.11.i; B.33.m.11.v; B.33.m.14.i; B.33.m.14.v;
B.33.m.15.i; B.33.m.15.v; B.33.m.18.i; B.33.m.18.v; B.33.m.25.i; B.33.m.25.v;
B.33.o.4.i; B.33.o.4.v; B.33.o.6.i; B.33.o.6.v; B.33.o.11.i; B.33.o.11.v; B.33.o.14.i;
B.33.o.14.v; B.33.o.15.i; B.33.o.15.v; B.33.o.18.i; B.33.o.18.v; B.33.o.25.i;
B.33.o.25.v; B.49.a.4.i; B.49.a.4.v; B.49.a.6.i; B.49.a.6.v; B.49.a.11.i; B.49.a.11.v;
B.49.a.14.i; B.49.a.14.v; B.49.a.15.i; B.49.a.15.v; B.49.a.18.i; B.49.a.18.v; B.49.a.25.i;
B.49.a.25.v; B.49.e.4.i; B.49.e.4.v; B.49.e.6.i; B.49.e.6.v; B.49.e.11.i; B.49.e.11.v;
B.49.e.14.i; B.49.e.14.v; B.49.e.15.i; B.49.e.15.v; B.49.e.18.i; B.49.e.18.v; B.49.e.25.i;
B.49.e.25.v; B.49.g.4.i; B.49.g.4.v; B.49.g.6.i; B.49.g.6.v; B.49.g.11.i; B.49.g.11.v;
B.49.g.14.i; B.49.g.14.v; B.49.g.15.i; B.49.g.15.v; B.49.g.18.i; B.49.g.18.v;
B.49.g.25.i; B.49.g.25.v; B.49.l.4.i; B.49.l.4.v; B.49.l.6.i; B.49.l.6.v; B.49.l.11.i;
B.49.l.11.v; B.49.l.14.i; B.49.l.14.v; B.49.l.15.i; B.49.l.15.v; B.49.l.18.i; B.49.l.18.v;
B.49.l.25.i; B.49.l.25.v; B.49.m.4.i; B.49.m.4.v; B.49.m.6.i; B.49.m.6.v; B.49.m.11.i;
B.49.m.11.v; B.49.m.14.i; B.49.m.14.v; B.49.m.15.i; B.49.m.15.v; B.49.m.18.i;
B.49.m.18.v; B.49.m.25.i; B.49.m.25.v; B.49.o.4.i; B.49.o.4.v; B.49.o.6.i; B.49.o.6.v;
B.49.o.11.i; B.49.o.11.v; B.49.o.14.i; B.49.o.14.v; B.49.o.15.i; B.49.o.15.v;
B.49.o.18.i; B.49.o.18.v; B.49.o.25.i; B.49.o.25.v; E.17.a.4.i; E.17.a.4.v; E.17.a.6.i;
E.17.a.6.v; E.17.a.11.i; E.17.a.11.v; E.17.a.14.i; E.17.a.14.v; E.17.a.15.i; E.17.a.15.v;
E.17.a.18.i; E.17.a.18.v; E.17.a.25.i; E.17.a.25.v; E.17.e.4.i; E.17.e.4.v; E.17.e.6.i;
E.17.e.6.v; E.17.e.11.i; E.17.e.11.v; E.17.e.14.i; E.17.e.14.v; E.17.e.15.i; E.17.e.15.v;
E.17.e.18.i; E.17.e.18.v; E.17.e.25.i; E.17.e.25.v; E.17.g.4.i; E.17.g.4.v; E.17.g.6.i;
E.17.g.6.v; E.17.g.11.i; E.17.g.11.v; E.17.g.14.i; E.17.g.14.v; E.17.g.15.i;
E.17.g.15.v; E.17.g.18.i; E.17.g.18.v; E.17.g.25.i; E.17.g.25.v; E.17.l.4.i; E.17.l.4.v;
E.17.l.6.i; E.17.l.6.v; E.17.l.11.i; E.17.l.11.v; E.17.l.14.i; E.17.l.14.v; E.17.l.15.i;
E.17.l.15.v; E.17.l.18.i; E.17.l.18.v; E.17.l.25.i; E.17.l.25.v; E.17.m.4.i; E.17.m.4.v;
E.17.m.6.i; E.17.m.6.v; E.17.m.11.i; E.17.m.11.v; E.17.m.14.i; E.17.m.14.v;
E.17.m.15.i; E.17.m.15.v; E.17.m.18.i; E.17.m.18.v; E.17.m.25.i; E.17.m.25.v;
E.17.o.4.i; E.17.o.4.v; E.17.o.6.i; E.17.o.6.v; E.17.o.11.i; E.17.o.11.v; E.17.o.14.i;
E.17.o.14.v; E.17.o.15.i; E.17.o.15.v; E.17.o.18.i; E.17.o.18.v; E.17.o.25.i;
E.17.o.25.v; E.33.a.4.i; E.33.a.4.v; E.33.a.6.i; E.33.a.6.v; E.33.a.11.i; E.33.a.11.v;
E.33.a.14.i; E.33.a.14.v; E.33.a.15.i; E.33.a.15.v; E.33.a.18.i; E.33.a.18.v; E.33.a.25.i;
E.33.a.25.v; E.33.e.4.i; E.33.e.4.v; E.33.e.6.i; E.33.e.6.v; E.33.e.11.i; E.33.e.11.v;
E.33.e.14.i; E.33.e.14.v; E.33.e.15.i; E.33.e.15.v; E.33.e.18.i; E.33.e.18.v; E.33.e.25.i;
E.33.e.25.v; E.33.g.4.i; E.33.g.4.v; E.33.g.6.i; E.33.g.6.v; E.33.g.11.i; E.33.g.11.v;
E.33.g.14.i; E.33.g.14.v; E.33.g.15.i; E.33.g.15.v; E.33.g.18.i; E.33.g.18.v;
E.33.g.25.i; E.33.g.25.v; E.33.l.4.i; E.33.l.4.v; E.33.l.6.i; E.33.l.6.v; E.33.l.11.i;
E.33.l.11.v; E.33.l.14.i; E.33.l.14.v; E.33.l.15.i; E.33.l.15.v; E.33.l.18.i; E.33.l.18.v;
E.33.l.25.i; E.33.l.25.v; E.33.m.4.i; E.33.m.4.v; E.33.m.6.i; E.33.m.6.v; E.33.m.11.i;
E.33.m.11.v; E.33.m.14.i; E.33.m.14.v; E.33.m.15.i; E.33.m.15.v; E.33.m.18.i;
E.33.m.18.v; E.33.m.25.i; E.33.m.25.v; E.33.o.4.i; E.33.o.4.v; E.33.o.6.i; E.33.o.6.v;
E.33.o.11.i; E.33.o.11.v; E.33.o.14.i; E.33.o.14.v; E.33.o.15.i; E.33.o.15.v;
E.33.o.18.i; E.33.o.18.v; E.33.o.25.i; E.33.o.25.v; E.49.a.4.i; E.49.a.4.v; E.49.a.6.i;
E.49.a.6.v; E.49.a.11.i; E.49.a.11.v; E.49.a.14.i; E.49.a.14.v; E.49.a.15.i; E.49.a.15.v;
E.49.a.18.i; E.49.a.18.v; E.49.a.25.i; E.49.a.25.v; E.49.e.4.i; E.49.e.4.v; E.49.e.6.i;
E.49.e.6.v; E.49.e.11.i; E.49.e.11.v; E.49.e.14.i; E.49.e.14.v; E.49.e.15.i; E.49.e.15.v;
E.49.e.18.i; E.49.e.18.v; E.49.e.25.i; E.49.e.25.v; E.49.g.4.i; E.49.g.4.v; E.49.g.6.i;
E.49.g.6.v; E.49.g.11.i; E.49.g.11.v; E.49.g.14.i; E.49.g.14.v; E.49.g.15.i;
E.49.g.15.v; E.49.g.18.i; E.49.g.18.v; E.49.g.25.i; E.49.g.25.v; E.49.l.4.i; E.49.l.4.v;
E.49.l.6.i; E.49.l.6.v; E.49.l.11.i; E.49.l.11.v; E.49.l.14.i; E.49.l.14.v; E.49.l.15.i;
E.49.l.15.v; E.49.l.18.i; E.49.l.18.v; E.49.l.25.i; E.49.l.25.v; E.49.m.4.i; E.49.m.4.v;
E.49.m.6.i; E.49.m.6.v; E.49.m.11.i; E.49.m.11.v; E.49.m.14.i; E.49.m.14.v;
E.49.m.15.i; E.49.m.15.v; E.49.m.18.i; E.49.m.18.v; E.49.m.25.i; E.49.m.25.v;
E.49.o.4.i; E.49.o.4.v; E.49.o.6.i; E.49.o.6.v; E.49.o.11.i; E.49.o.11.v; E.49.o.14.i;
E.49.o.14.v; E.49.o.15.i; E.49.o.15.v; E.49.o.18.i; E.49.o.18.v; E.49.o.25.i;
E.49.o.25.v; H.17.a.4.i; H.17.a.4.v; H.17.a.6.i; H.17.a.6.v; H.17.a.11.i; H.17.a.11.v;
H.17.a.14.i; H.17.a.14.v; H.17.a.15.i; H.17.a.15.v; H.17.a.18.i; H.17.a.18.v;
H.17.a.25.i; H.17.a.25.v; H.17.e.4.i; H.17.e.4.v; H.17.e.6.i; H.17.e.6.v; H.17.e.11.i;
H.17.e.11.v; H.17.e.14.i; H.17.e.14.v; H.17.e.15.i; H.17.e.15.v; H.17.e.18.i;
H.17.e.18.v; H.17.e.25.i; H.17.e.25.v; H.17.g.4.i; H.17.g.4.v; H.17.g.6.i; H.17.g.6.v;
H.17.g.11.i; H.17.g.11.v; H.17.g.14.i; H.17.g.14.v; H.17.g.15.i; H.17.g.15.v;
H.17.g.18.i; H.17.g.18.v; H.17.g.25.i; H.17.g.25.v; H.17.l.4.i; H.17.l.4.v; H.17.l.6.i;
H.17.l.6.v; H.17.l.11.i; H.17.l.11.v; H.17.l.14.i; H.17.l.14.v; H.17.l.15.i; H.17.l.15.v;
H.17.l.18.i; H.17.l.18.v; H.17.l.25.i; H.17.l.25.v; H.17.m.4.i; H.17.m.4.v;
H.17.m.6.i; H.17.m.6.v; H.17.m.11.i; H.17.m.11.v; H.17.m.14.i; H.17.m.14.v;
H.17.m.15.i; H.17.m.15.v; H.17.m.18.i; H.17.m.18.v; H.17.m.25.i; H.17.m.25.v;
H.17.o.4.i; H.17.o.4.v; H.17.o.6.i; H.17.o.6.v; H.17.o.11.i; H.17.o.11.v; H.17.o.14.i;
H.17.o.14.v; H.17.o.15.i; H.17.o.15.v; H.17.o.18.i; H.17.o.18.v; H.17.o.25.i;
H.17.o.25.v; H.33.a.4.i; H.33.a.4.v; H.33.a.6.i; H.33.a.6.v; H.33.a.11.i; H.33.a.11.v;
H.33.a.14.i; H.33.a.14.v; H.33.a.15.i; H.33.a.15.v; H.33.a.18.i; H.33.a.18.v;
H.33.a.25.i; H.33.a.25.v; H.33.e.4.i; H.33.e.4.v; H.33.e.6.i; H.33.e.6.v; H.33.e.11.i;
H.33.e.11.v; H.33.e.14.i; H.33.e.14.v; H.33.e.15.i; H.33.e.15.v; H.33.e.18.i;
H.33.e.18.v; H.33.e.25.i; H.33.e.25.v; H.33.g.4.i; H.33.g.4.v; H.33.g.6.i; H.33.g.6.v;
H.33.g.11.i; H.33.g.11.v; H.33.g.14.i; H.33.g.14.v; H.33.g.15.i; H.33.g.15.v;
H.33.g.18.i; H.33.g.18.v; H.33.g.25.i; H.33.g.25.v; H.33.l.4.i; H.33.l.4.v; H.33.l.6.i;
H.33.l.6.v; H.33.l.11.i; H.33.l.11.v; H.33.l.14.i; H.33.l.14.v; H.33.l.15.i; H.33.l.15.v;
H.33.l.18.i; H.33.l.18.v; H.33.l.25.i; H.33.l.25.v; H.33.m.4.i; H.33.m.4.v;
H.33.m.6.i; H.33.m.6.v; H.33.m.11.i; H.33.m.11.v; H.33.m.14.i; H.33.m.14.v;
H.33.m.15.i; H.33.m.15.v; H.33.m.18.i; H.33.m.18.v; H.33.m.25.i; H.33.m.25.v;
H.33.o.4.i; H.33.o.4.v; H.33.o.6.i; H.33.o.6.v; H.33.o.11.i; H.33.o.11.v; H.33.o.14.i;
H.33.o.14.v; H.33.o.15.i; H.33.o.15.v; H.33.o.18.i; H.33.o.18.v; H.33.o.25.i;
H.33.o.25.v; H.49.a.4.i; H.49.a.4.v; H.49.a.6.i; H.49.a.6.v; H.49.a.11.i; H.49.a.11.v;
H.49.a.14.i; H.49.a.14.v; H.49.a.15.i; H.49.a.15.v; H.49.a.18.i; H.49.a.18.v;
H.49.a.25.i; H.49.a.25.v; H.49.e.4.i; H.49.e.4.v; H.49.e.6.i; H.49.e.6.v; H.49.e.11.i;
H.49.e.11.v; H.49.e.14.i; H.49.e.14.v; H.49.e.15.i; H.49.e.15.v; H.49.e.18.i;
H.49.e.18.v; H.49.e.25.i; H.49.e.25.v; H.49.g.4.i; H.49.g.4.v; H.49.g.6.i; H.49.g.6.v;
H.49.g.11.i; H.49.g.11.v; H.49.g.14.i; H.49.g.14.v; H.49.g.15.i; H.49.g.15.v;
H.49.g.18.i; H.49.g.18.v; H.49.g.25.i; H.49.g.25.v; H.49.l.4.i; H.49.l.4.v; H.49.l.6.i;
H.49.l.6.v; H.49.l.11.i; H.49.l.11.v; H.49.l.14.i; H.49.l.14.v; H.49.l.15.i; H.49.l.15.v;
H.49.l.18.i; H.49.l.18.v; H.49.l.25.i; H.49.l.25.v; H.49.m.4.i; H.49.m.4.v;
H.49.m.6.i; H.49.m.6.v; H.49.m.11.i; H.49.m.11.v; H.49.m.14.i; H.49.m.14.v;
H.49.m.15.i; H.49.m.15.v; H.49.m.18.i; H.49.m.18.v; H.49.m.25.i; H.49.m.25.v;
H.49.o.4.i; H.49.o.4.v; H.49.o.6.i; H.49.o.6.v; H.49.o.11.i; H.49.o.11.v; H.49.o.14.i;
H.49.o.14.v; H.49.o.15.i; H.49.o.15.v; H.49.o.18.i; H.49.o.18.v; H.49.o.25.i;
H.49.o.25.v; I.17.a.4.i; I.17.a.4.v; I.17.a.6.i; I.17.a.6.v; I.17.a.11.i; I.17.a.11.v;
I.17.a.14.i; I.17.a.14.v; I.17.a.15.i; I.17.a.15.v; I.17.a.18.i; I.17.a.18.v; I.17.a.25.i;
I.17.a.25.v; I.17.e.4.i; I.17.e.4.v; I.17.e.6.i; I.17.e.6.v; I.17.e.11.i; I.17.e.11.v;
I.17.e.14.i; I.17.e.14.v; I.17.e.15.i; I.17.e.15.v; I.17.e.18.i; I.17.e.18.v; I.17.e.25.i;
I.17.e.25.v; I.17.g.4.i; I.17.g.4.v; I.17.g.6.i; I.17.g.6.v; I.17.g.11.i; I.17.g.11.v;
I.17.g.14.i; I.17.g.14.v; I.17.g.15.i; I.17.g.15.v; I.17.g.18.i; I.17.g.18.v; I.17.g.25.i;
I.17.g.25.v; I.17.l.4.i; I.17.l.4.v; I.17.l.6.i; I.17.l.6.v; I.17.l.11.i; I.17.l.11.v; I.17.l.14.i;
I.17.l.14.v; I.17.l.15.i; I.17.l.15.v; I.17.l.18.i; I.17.l.18.v; I.17.l.25.i; I.17.l.25.v;
I.17.m.4.i; I.17.m.4.v; I.17.m.6.i; I.17.m.6.v; I.17.m.11.i; I.17.m.11.v; I.17.m.14.i;
I.17.m.14.v; I.17.m.15.i; I.17.m.15.v; I.17.m.18.i; I.17.m.18.v; I.17.m.25.i;
I.17.m.25.v; I.17.o.4.i; I.17.o.4.v; I.17.o.6.i; I.17.o.6.v; I.17.o.11.i; I.17.o.11.v;
I.17.o.14.i; I.17.o.14.v; I.17.o.15.i; I.17.o.15.v; I.17.o.18.i; I.17.o.18.v; I.17.o.25.i;
I.17.o.25.v; I.33.a.4.i; I.33.a.4.v; I.33.a.6.i; I.33.a.6.v; I.33.a.11.i; I.33.a.11.v;
I.33.a.14.i; I.33.a.14.v; I.33.a.15.i; I.33.a.15.v; I.33.a.18.i; I.33.a.18.v; I.33.a.25.i;
I.33.a.25.v; I.33.e.4.i; I.33.e.4.v; I.33.e.6.i; I.33.e.6.v; I.33.e.11.i; I.33.e.11.v;
I.33.e.14.i; I.33.e.14.v; I.33.e.15.i; I.33.e.15.v; I.33.e.18.i; I.33.e.18.v; I.33.e.25.i;
I.33.e.25.v; I.33.g.4.i; I.33.g.4.v; I.33.g.6.i; I.33.g.6.v; I.33.g.11.i; I.33.g.11.v;
I.33.g.14.i; I.33.g.14.v; I.33.g.15.i; I.33.g.15.v; I.33.g.18.i; I.33.g.18.v; I.33.g.25.i;
I.33.g.25.v; I.33.l.4.i; I.33.l.4.v; I.33.l.6.i; I.33.l.6.v; I.33.l.11.i; I.33.l.11.v; I.33.l.14.i;
I.33.l.14.v; I.33.l.15.i; I.33.l.15.v; I.33.l.18.i; I.33.l.18.v; I.33.l.25.i; I.33.l.25.v;
I.33.m.4.i; I.33.m.4.v; I.33.m.6.i; I.33.m.6.v; I.33.m.11.i; I.33.m.11.v; I.33.m.14.i;
I.33.m.14.v; I.33.m.15.i; I.33.m.15.v; I.33.m.18.i; I.33.m.18.v; I.33.m.25.i;
I.33.m.25.v; I.33.o.4.i; I.33.o.4.v; I.33.o.6.i; I.33.o.6.v; I.33.o.11.i; I.33.o.11.v;
I.33.o.14.i; I.33.o.14.v; I.33.o.15.i; I.33.o.15.v; I.33.o.18.i; I.33.o.18.v; I.33.o.25.i;
I.33.o.25.v; I.49.a.4.i; I.49.a.4.v; I.49.a.6.i; I.49.a.6.v; I.49.a.11.i; I.49.a.11.v;
I.49.a.14.i; I.49.a.14.v; I.49.a.15.i; I.49.a.15.v; I.49.a.18.i; I.49.a.18.v; I.49.a.25.i;
I.49.a.25.v; I.49.e.4.i; I.49.e.4.v; I.49.e.6.i; I.49.e.6.v; I.49.e.11.i; I.49.e.11.v;
I.49.e.14.i; I.49.e.14.v; I.49.e.15.i; I.49.e.15.v; I.49.e.18.i; I.49.e.18.v; I.49.e.25.i;
I.49.e.25.v; I.49.g.4.i; I.49.g.4.v; I.49.g.6.i; I.49.g.6.v; I.49.g.11.i; I.49.g.11.v;
I.49.g.14.i; I.49.g.14.v; I.49.g.15.i; I.49.g.15.v; I.49.g.18.i; I.49.g.18.v; I.49.g.25.i;
I.49.g.25.v; I.49.l.4.i; I.49.l.4.v; I.49.l.6.i; I.49.l.6.v; I.49.l.11.i; I.49.l.11.v; I.49.l.14.i;
I.49.l.14.v; I.49.l.15.i; I.49.l.15.v; I.49.l.18.i; I.49.l.18.v; I.49.l.25.i; I.49.l.25.v;
I.49.m.4.i; I.49.m.4.v; I.49.m.6.i; I.49.m.6.v; I.49.m.11.i; I.49.m.11.v; I.49.m.14.i;
I.49.m.14.v; I.49.m.15.i; I.49.m.15.v; I.49.m.18.i; I.49.m.18.v; I.49.m.25.i;
I.49.m.25.v; I.49.o.4.i; I.49.o.4.v; I.49.o.6.i; I.49.o.6.v; I.49.o.11.i; I.49.o.11.v;
I.49.o.14.i; I.49.o.14.v; I.49.o.15.i; I.49.o.15.v; I.49.o.18.i; I.49.o.18.v; I.49.o.25.i;
I.49.o.25.v; L.17.a.4.i; L.17.a.4.v; L.17.a.6.i; L.17.a.6.v; L.17.a.11.i; L.17.a.11.v;
L.17.a.14.i; L.17.a.14.v; L.17.a.15.i; L.17.a.15.v; L.17.a.18.i; L.17.a.18.v; L.17.a.25.i;
L.17.a.25.v; L.17.e.4.i; L.17.e.4.v; L.17.e.6.i; L.17.e.6.v; L.17.e.11.i; L.17.e.11.v;
L.17.e.14.i; L.17.e.14.v; L.17.e.15.i; L.17.e.15.v; L.17.e.18.i; L.17.e.18.v; L.17.e.25.i;
L.17.e.25.v; L.17.g.4.i; L.17.g.4.v; L.17.g.6.i; L.17.g.6.v; L.17.g.11.i; L.17.g.11.v;
L.17.g.14.i; L.17.g.14.v; L.17.g.15.i; L.17.g.15.v; L.17.g.18.i; L.17.g.18.v;
L.17.g.25.i; L.17.g.25.v; L.17.l.4.i; L.17.l.4.v; L.17.l.6.i; L.17.l.6.v; L.17.l.11.i;
L.17.l.11.v; L.17.l.14.i; L.17.l.14.v; L.17.l.15.i; L.17.l.15.v; L.17.l.18.i; L.17.l.18.v;
L.17.l.25.i; L.17.l.25.v; L.17.m.4.i; L.17.m.4.v; L.17.m.6.i; L.17.m.6.v; L.17.m.11.i;
L.17.m.11.v; L.17.m.14.i; L.17.m.14.v; L.17.m.15.i; L.17.m.15.v; L.17.m.18.i;
L.17.m.18.v; L.17.m.25.i; L.17.m.25.v; L.17.o.4.i; L.17.o.4.v; L.17.o.6.i; L.17.o.6.v;
L.17.o.11.i; L.17.o.11.v; L.17.o.14.i; L.17.o.14.v; L.17.o.15.i; L.17.o.15.v;
L.17.o.18.i; L.17.o.18.v; L.17.o.25.i; L.17.o.25.v; L.33.a.4.i; L.33.a.4.v; L.33.a.6.i;
L.33.a.6.v; L.33.a.11.i; L.33.a.11.v; L.33.a.14.i; L.33.a.14.v; L.33.a.15.i; L.33.a.15.v;
L.33.a.18.i; L.33.a.18.v; L.33.a.25.i; L.33.a.25.v; L.33.e.4.i; L.33.e.4.v; L.33.e.6.i;
L.33.e.6.v; L.33.e.11.i; L.33.e.11.v; L.33.e.14.i; L.33.e.14.v; L.33.e.15.i; L.33.e.15.v;
L.33.e.18.i; L.33.e.18.v; L.33.e.25.i; L.33.e.25.v; L.33.g.4.i; L.33.g.4.v; L.33.g.6.i;
L.33.g.6.v; L.33.g.11.i; L.33.g.11.v; L.33.g.14.i; L.33.g.14.v; L.33.g.15.i;
L.33.g.15.v; L.33.g.18.i; L.33.g.18.v; L.33.g.25.i; L.33.g.25.v; L.33.l.4.i; L.33.l.4.v;
L.33.l.6.i; L.33.l.6.v; L.33.l.11.i; L.33.l.11.v; L.33.l.14.i; L.33.l.14.v; L.33.l.15.i;
L.33.l.15.v; L.33.l.18.i; L.33.l.18.v; L.33.l.25.i; L.33.l.25.v; L.33.m.4.i; L.33.m.4.v;
L.33.m.6.i; L.33.m.6.v; L.33.m.11.i; L.33.m.11.v; L.33.m.14.i; L.33.m.14.v;
L.33.m.15.i; L.33.m.15.v; L.33.m.18.i; L.33.m.18.v; L.33.m.25.i; L.33.m.25.v;
L.33.o.4.i; L.33.o.4.v; L.33.o.6.i; L.33.o.6.v; L.33.o.11.i; L.33.o.11.v; L.33.o.14.i;
L.33.o.14.v; L.33.o.15.i; L.33.o.15.v; L.33.o.18.i; L.33.o.18.v; L.33.o.25.i;
L.33.o.25.v; L.49.a.4.i; L.49.a.4.v; L.49.a.6.i; L.49.a.6.v; L.49.a.11.i; L.49.a.11.v;
L.49.a.14.i; L.49.a.14.v; L.49.a.15.i; L.49.a.15.v; L.49.a.18.i; L.49.a.18.v; L.49.a.25.i;
L.49.a.25.v; L.49.e.4.i; L.49.e.4.v; L.49.e.6.i; L.49.e.6.v; L.49.e.11.i; L.49.e.11.v;
L.49.e.14.i; L.49.e.14.v; L.49.e.15.i; L.49.e.15.v; L.49.e.18.i; L.49.e.18.v; L.49.e.25.i;
L.49.e.25.v; L.49.g.4.i; L.49.g.4.v; L.49.g.6.i; L.49.g.6.v; L.49.g.11.i; L.49.g.11.v;
L.49.g.14.i; L.49.g.14.v; L.49.g.15.i; L.49.g.15.v; L.49.g.18.i; L.49.g.18.v;
L.49.g.25.i; L.49.g.25.v; L.49.l.4.i; L.49.l.4.v; L.49.l.6.i; L.49.l.6.v; L.49.l.11.i;
L.49.l.11.v; L.49.l.14.i; L.49.l.14.v; L.49.l.15.i; L.49.l.15.v; L.49.l.18.i; L.49.l.18.v;
L.49.l.25.i; L.49.l.25.v; L.49.m.4.i; L.49.m.4.v; L.49.m.6.i; L.49.m.6.v; L.49.m.11.i;
L.49.m.11.v; L.49.m.14.i; L.49.m.14.v; L.49.m.15.i; L.49.m.15.v; L.49.m.18.i;
L.49.m.18.v; L.49.m.25.i; L.49.m.25.v; L.49.o.4.i; L.49.o.4.v; L.49.o.6.i; L.49.o.6.v;
L.49.o.11.i; L.49.o.11.v; L.49.o.14.i; L.49.o.14.v; L.49.o.15.i; L.49.o.15.v;
L.49.o.18.i; L.49.o.18.v; L.49.o.25.i; L.49.o.25.v; B.93.a.4.i; B.93.a.4.v; B.93.a.6.i;
B.93.a.6.v; B.93.a.11.i; B.93.a.11.v; B.93.a.14.i; B.93.a.14.v; B.93.a.15.i; B.93.a.15.v;
B.93.a.18.i; B.93.a.18.v; B.93.a.25.i; B.93.a.25.v; B.93.e.4.i; B.93.e.4.v; B.93.e.6.i;
B.93.e.6.v; B.93.e.11.i; B.93.e.11.v; B.93.e.14.i; B.93.e.14.v; B.93.e.15.i; B.93.e.15.v;
B.93.e.18.i; B.93.e.18.v; B.93.e.25.i; B.93.e.25.v; B.93.g.4.i; B.93.g.4.v; B.93.g.6.i;
B.93.g.6.v; B.93.g.11.i; B.93.g.11.v; B.93.g.14.i; B.93.g.14.v; B.93.g.15.i;
B.93.g.15.v; B.93.g.18.i; B.93.g.18.v; B.93.g.25.i; B.93.g.25.v; B.93.l.4.i; B.93.l.4.v;
B.93.l.6.i; B.93.l.6.v; B.93.l.11.i; B.93.l.11.v; B.93.l.14.i; B.93.l.14.v; B.93.l.15.i;
B.93.l.15.v; B.93.l.18.i; B.93.l.18.v; B.93.l.25.i; B.93.l.25.v; B.93.m.4.i; B.93.m.4.v;
B.93.m.6.i; B.93.m.6.v; B.93.m.11.i; B.93.m.11.v; B.93.m.14.i; B.93.m.14.v;
B.93.m.15.i; B.93.m.15.v; B.93.m.18.i; B.93.m.18.v; B.93.m.25.i; B.93.m.25.v;
B.93.o.4.i; B.93.o.4.v; B.93.o.6.i; B.93.o.6.v; B.93.o.11.i; B.93.o.11.v; B.93.o.14.i;
B.93.o.14.v; B.93.o.15.i; B.93.o.15.v; B.93.o.18.i; B.93.o.18.v; B.93.o.25.i;
B.93.o.25.v; B.94.a.4.i; B.94.a.4.v; B.94.a.6.i; B.94.a.6.v; B.94.a.11.i; B.94.a.11.v;
B.94.a.14.i; B.94.a.14.v; B.94.a.15.i; B.94.a.15.v; B.94.a.18.i; B.94.a.18.v; B.94.a.25.i;
B.94.a.25.v; B.94.e.4.i; B.94.e.4.v; B.94.e.6.i; B.94.e.6.v; B.94.e.11.i; B.94.e.11.v;
B.94.e.14.i; B.94.e.14.v; B.94.e.15.i; B.94.e.15.v; B.94.e.18.i; B.94.e.18.v; B.94.e.25.i;
B.94.e.25.v; B.94.g.4.i; B.94.g.4.v; B.94.g.6.i; B.94.g.6.v; B.94.g.11.i; B.94.g.11.v;
B.94.g.14.i; B.94.g.14.v; B.94.g.15.i; B.94.g.15.v; B.94.g.18.i; B.94.g.18.v;
B.94.g.25.i; B.94.g.25.v; B.94.l.4.i; B.94.l.4.v; B.94.l.6.i; B.94.l.6.v; B.94.l.11.i;
B.94.l.11.v; B.94.l.14.i; B.94.l.14.v; B.94.l.15.i; B.94.l.15.v; B.94.l.18.i; B.94.l.18.v;
B.94.l.25.i; B.94.l.25.v; B.94.m.4.i; B.94.m.4.v; B.94.m.6.i; B.94.m.6.v; B.94.m.11.i;
B.94.m.11.v; B.94.m.14.i; B.94.m.14.v; B.94.m.15.i; B.94.m.15.v; B.94.m.18.i;
B.94.m.18.v; B.94.m.25.i; B.94.m.25.v; B.94.o.4.i; B.94.o.4.v; B.94.o.6.i; B.94.o.6.v;
B.94.o.11.i; B.94.o.11.v; B.94.o.14.i; B.94.o.14.v; B.94.o.15.i; B.94.o.15.v;
B.94.o.18.i; B.94.o.18.v; B.94.o.25.i; B.94.o.25.v; E.93.a.4.i; E.93.a.4.v; E.93.a.6.i;
E.93.a.6.v; E.93.a.11.i; E93.a.11.v; E.93.a.14.i; E.93.a.14.v; E.93.a.15.i; E.93.a.15.v;
E.93.a.18.i; E.93.a.18.v; E.93.a.25.i; E.93.a.25.v; E.93.e.4.i; E.93.e.4.v; E.93.e.6.i;
E.93.e.6.v; E.93.e.11.i; E.93.e.11.v; E.93.e.14.i; E.93.e.14.v; E.93.e.15.i; E.93.e.15.v;
E.93.e.18.i; E.93.e.18.v; E.93.e.25.i; E.93.e.25.v; E.93.g.4.i; E.93.g.4.v; E.93.g.6.i;
E.93.g.6.v; E.93.g.11.i; E.93.g.11.v; E.93.g.14.i; E.93.g.14.v; E.93.g.15.i;
E.93.g.15.v; E.93.g.18.i; E.93.g.18.v; E.93.g.25.i; E.93.g.25.v; E.93.l.4.i; E.93.l.4.v;
E.93.l.6.i; E.93.l.6.v; E.93.l.11.i; E.93.l.11.v; E.93.l.14.i; E.93.l.14.v; E.93.l.15.i;
E.93.l.15.v; E.93.l.18.i; E.93.l.18.v; E.93.l.25.i; E.93.l.25.v; E.93.m.4.i; E.93.m.4.v;
E.93.m.6.i; E.93.m.6.v; E.93.m.11.i; E.93.m.11.v; E.93.m.14.i; E.93.m.14.v;
E.93.m.15.i; E.93.m.15.v; E.93.m.18.i; E.93.m.18.v; E.93.m.25.i; E.93.m.25.v;
E.93.o.4.i; E.93.o.4.v; E.93.o.6.i; E.93.o.6.v; E.93.o.11.i; E.93.o.11.v; E.93.o.14.i;
E.93.o.14.v; E.93.o.15.i; E.93.o.15.v; E.93.o.18.i; E.93.o.18.v; E.93.o.25.i;
E.93.o.25.v; E.94.a.4.i; E.94.a.4.v; E.94.a.6.i; E.94.a.6.v; E.94.a.11.i; E.94.a.11.v;
E.94.a.14.i; E.94.a.14.v; E.94.a.15.i; E.94.a.15.v; E.94.a.18.i; E.94.a.18.v; E.94.a.25.i;
E.94.a.25.v; E.94.e.4.i; E.94.e.4.v; E.94.e.6.i; E.94.e.6.v; E.94.e.11.i; E.94.e.11.v;
E.94.e.14.i; E.94.e.14.v; E.94.e.15.i; E.94.e.15.v; E.94.e.18.i; E.94.e.18.v; E.94.e.25.i;
E.94.e.25.v; E.94.g.4.i; E.94.g.4.v; E.94.g.6.i; E.94.g.6.v; E.94.g.11.i; E.94.g.11.v;
E.94.g.14.i; E.94.g.14.v; E.94.g.15.i; E.94.g.15.v; E.94.g.18.i; E.94.g.18.v;
E.94.g.25.i; E.94.g.25.v; E.94.l.4.i; E.94.l.4.v; E.94.l.6.i; E.94.l.6.v; E.94.l.11.i;
E.94.l.11.v; E.94.l.14.i; E.94.l.14.v; E.94.l.15.i; E.94.l.15.v; E.94.l.18.i; E.94.l.18.v;
E.94.l.25.i; E.94.l.25.v; E.94.m.4.i; E.94.m.4.v; E.94.m.6.i; E.94.m.6.v; E.94.m.11.i;
E.94.m.11.v; E.94.m.14.i; E.94.m.14.v; E.94.m.15.i; E.94.m.15.v; E.94.m.18.i;
E.94.m.18.v; E.94.m.25.i; E.94.m.25.v; E.94.o.4.i; E.94.o.4.v; E.94.o.6.i; E.94.o.6.v;
E.94.o.11.i; E.94.o.11.v; E.94.o.14.i; E.94.o.14.v; E.94.o.15.i; E.94.o.15.v;
E.94.o.18.i; E.94.o.18.v; E.94.o.25.i; E.94.o.25.v; I.93.a.4.i; I.93.a.4.v; I.93.a.6.i;
I.93.a.6.v; I.93.a.11.i; I.93.a.11.v; I.93.a.14.i; I.93.a.14.v; I.93.a.15.i; I.93.a.15.v;
I.93.a.18.i; I.93.a.18.v; I.93.a.25.i; I.93.a.25.v; I.93.e.4.i; I.93.e.4.v; I.93.e.6.i;
I.93.e.6.v; I.93.e.11.i; I.93.e.11.v; I.93.e.14.i; I.93.e.14.v; I.93.e.15.i; I.93.e.15.v;
I.93.e.18.i; I.93.e.18.v; I.93.e.25.i; I.93.e.25.v; I.93.g.4.i; I.93.g.4.v; I.93.g.6.i;
I.93.g.6.v; I.93.g.11.i; I.93.g.11.v; I.93.g.14.i; I.93.g.14.v; I.93.g.15.i; I.93.g.15.v;
I.93.g.18.i; I.93.g.18.v; I.93.g.25.i; I.93.g.25.v; I.93.l.4.i; I.93.l.4.v; I.93.l.6.i;
I.93.l.6.v; 1.93.l.11.i; I.93.l.11.v; I.93.l.14.i; I.93.l.14.v; I.93.l.15.i; I.93.l.15.v;
I.93.l.18.i; I.93.l.18.v; I.93.l.25.i; I.93.l.25.v; I.93.m.4.i; I.93.m.4.v; I.93.m.6.i;
I.93.m.6.v; I.93.m.11.i; I.93.m.11.v; I.93.m.14.i; I.93.m.14.v; I.93.m.15.i;
I.93.m.15.v; I.93.m.18.i; I.93.m.18.v; I.93.m.25.i; I.93.m.25.v; I.93.o.4.i; I.93.o.4.v;
I.93.o.6.i; I.93.o.6.v; I.93.o.11.i; I.93.o.11.v; I.93.0.14.i; I.93.o.14.v; I.93.o.15.i;
I.93.o.15.v; I.93.o.18.i; I.93.o.18.v; I.93.o.25.i; I.93.o.25.v; I.94.a.4.i; I.94.a.4.v;
I.94.a.6.i; I.94.a.6.v; I.94.a.11.i; I.94.a.11.v; I.94.a.14.i; I.94.a.14.v; I.94.a.15.i;
I.94.a.15.v; I.94.a.18.i; I.94.a.18.v; I.94.a.25.i; I.94.a.25.v; I.94.e.4.i; I.94.e.4.v;
I.94.e.6.i; I.94.e.6.v; I.94.e.11.i; I.94.e.11.v; I.94.e.14.i; I.94.e.14.v; I.94.e.15.i;
I.94.e.15.v; I.94.e.18.i; I.94.e.18.v; I.94.e.25.i; 1.94.e.25.v; I.94.g.4.i; I.94.g.4.v;
I.94.g.6.i; I.94.g.6.v; I.94.g.11.i; I.94.g.11.v; I.94.g.14.i; I.94.g.14.v; I.94.g.15.i;
I.94.g.15.v; I.94.g.18.i; I.94.g.18.v; I.94.g.25.i; I.94.g.25.v; I.94.l.4.i; I.94.l.4.v;
I.94.l.6.i; I.94.l.6.v; I.94.l.11.i; I.94.l.11.v; I.94.l.14.i; I.94.l.14.v; I.94.l.15.i;
I.94.l.15.v; I.94.l.18.i; I.94.l.18.v; I.94.l.25.i; I.94.l.25.v; I.94.m.4.i; I.94.m.4.v;
I.94.m.6.i; I.94.m.6.v; I.94.m.11.i; I.94.m.11.v; I.94.m.14.i; I.94.m.14.v; I.94.m.15.i;
I.94.m.15.v; I.94.m.18.i; I.94.m.18.v; I.94.m.25.i; I.94.m.25.v; I.94.o.4.i; I.94.o.4.v;
I.94.o.6.i; I.94.o.6.v; I.94.o.11.i; I.94.o.11.v; I.94.o.14.i; I.94.o.14.v; I.94.o.15.i;
I.94.o.15.v; I.94.o.18.i; I.94.o.18.v; I.94.o.25.i; I.94.o.25.v; L.93.a.4.i; L.93.a.4.v;
L.93.a.6.i; L.93.a.6.v; L.93.a.11.i; L.93.a.11.v; L.93.a.14.i; L.93.a.14.v; L.93.a.15.i;
L.93.a.15.v; L.93.a.18.i; L.93.a.18.v; L.93.a.25.i; L.93.a.25.v; L.93.e.4.i; L.93.e.4.v;
L.93.e.6.i; L.93.e.6.v; L.93.e.11.i; L.93.e.11.v; L.93.e.14.i; L.93.e.14.v; L.93.e.15.i;
L.93.e.15.v; L.93.e.18.i; L.93.e.18.v; L.93.e.25.i; L.93.e.25.v; L.93.g.4.i; L.93.g.4.v;
L.93.g.6.i; L.93.g.6.v; L.93.g.11.i; L.93.g.11.v; L.93.g.14.i; L.93.g.14.v; L.93.g.15.i;
L.93.g.15.v; L.93.g.18.i; L.93.g.18.v; L.93.g.25.i; L.93.g.25.v; L.93.l.4.i; L.93.l.4.v;
L.93.l.6.i; L.93.l.6.v; L.93.l.11.i; L.93.l.11.v; L.93.l.14.i; L.93.l.14.v; L.93.l.15.i;
L.93.l.15.v; L.93.l.18.i; L.93.l.18.v; L.93.l.25.i; L.93.l.25.v; L.93.m.4.i; L.93.m.4.v;
L.93.m.6.i; L.93.m.6.v; L.93.m.11.i; L.93.m.11.v; L.93.m.14.i; L.93.m.14.v;
L.93.m.15.i; L.93.m.15.v; L.93.m.18.i; L.93.m.18.v; L.93.m.25.i; L.93.m.25.v;
L.93.o.4.i; L.93.o.4.v; L.93.o.6.i; L.93.o.6.v; L.93.o.11.i; L.93.o.11.v; L.93.o.14.i;
L.93.o.14.v; L.93.o.15.i; L.93.o.15.v; L.93.o.18.i; L.93.o.18.v; L.93.o.25.i;
L.93.o.25.v; L.94.a.4.i; L.94.a.4.v; L.94.a.6.i; L.94.a.6.v; L.94.a.11.i; L.94.a.11.v;
L.94.a.14.i; L.94.a.14.v; L.94.a.15.i; L.94.a.15.v; L.94.a.18.i; L.94.a.18.v; L.94.a.25.i;
L.94.a.25.v; L.94.e.4.i; L.94.e.4.v; L.94.e.6.i; L.94.e.6.v; L.94.e.11.i; L.94.e.11.v;
L.94.e.14.i; L.94.e.14.v; L.94.e.15.i; L.94.e.15.v; L.94.e.18.i; L.94.e.18.v; L.94.e.25.i;
L.94.e.25.v; L.94.g.4.i; L.94.g.4.v; L.94.g.6.i; L.94.g.6.v; L.94.g.11.i; L.94.g.11.v;
L.94.g.14.i; L.94.g.14.v; L.94.g.15.i; L.94.g.15.v; L.94.g.18.i; L.94.g.18.v;
L.94.g.25.i; L.94.g.25.v; L.94.l.4.i; L.94.l.4.v; L.94.l.6.i; L.94.l.6.v; L.94.l.11.i;
L.94.l.11.v; L.94.l.14.i; L.94.l.14.v; L.94.l.15.i; L.94.l.15.v; L.94.l.18.i; L.94.l.18.v;
L.94.l.25.i; L.94.l.25.v; L.94.m.4.i; L.94.m.4.v; L.94.m.6.i; L.94.m.6.v; L.94.m.11.i;
L.94.m.11.v; L.94.m.14.i; L.94.m.14.v; L.94.m.15.i; L.94.m.15.v; L.94.m.18.i;
L.94.m.18.v; L.94.m.25.i; L.94.m.25.v; L.94.o.4.i; L.94.o.4.v; L.94.o.6.i; L.94.o.6.v;
L.94.o.11.i; L.94.o.11.v; L.94.o.14.i; L.94.o.14.v; L.94.o.15.i; L.94.o.15.v;
L.94.o.18.i; L.94.o.18.v; L.94.o.25.i; L.94.o.25.v; O.93.a.4.i; O.93.a.4.v; O.93.a.6.i;
O.93.a.6.v; O.93.a.11.i; O.93.a.11.v; O.93.a.14.i; O.93.a.14.v; O.93.a.15.i;
O.93.a.15.v; O.93.a.18.i; O.93.a.18.v; O.93.a.25.i; O.93.a.25.v; O.93.e.4.i;
O.93.e.4.v; O.93.e.6.i; O.93.e.6.v; O.93.e.11.i; O.93.e.11.v; O.93.e.14.i; O.93.e.14.v;
O.93.e.15.i; O.93.e.15.v; O.93.e.18.i; O.93.e.18.v; O.93.e.25.i; O.93.e.25.v;
O.93.g.4.i; O.93.g.4.v; O.93.g.6.i; O.93.g.6.v; O.93.g.11.i; O.93.g.11.v; O.93.g.14.i;
O.93.g.14.v; O.93.g.15.i; O.93.g.15.v; O.93.g.18.i; O.93.g.18.v; O.93.g.25.i;
O.93.g.25.v; O.93.l.4.i; O.93.l.4.v; O.93.l.6.i; O.93.l.6.v; O.93.l.11.i; O.93.l.11.v;
O.93.l.14.i; O.93.l.14.v; O.93.l.15.i; O.93.l.15.v; O.93.l.18.i; O.93.l.18.v; O.93.l.25.i;
O.93.l.25.v; O.93.m.4.i; O.93.m.4.v; O.93.m.6.i; O.93.m.6.v; O.93.m.11.i;
O.93.m.11.v; O.93.m.14.i; O.93.m.14.v; O.93.m.15.i; O.93.m.15.v; O.93.m.18.i;
O.93.m.18.v; O.93.m.25.i; O.93.m.25.v; O.93.o.4.i; O.93.o.4.v; O.93.o.6.i;
O.93.o.6.v; O.93.o.11.i; O.93.o.11.v; O.93.o.14.i; O.93.o.14.v; O.93.o.15.i;
O.93.o.15.v; O.93.o.18.i; O.93.o.18.v; O.93.o.25.i; O.93.o.25.v; O.94.a.4.i;
O.94.a.4.v; O.94.a.6.i; O.94.a.6.v; O.94.a.11.i; O.94.a.11.v; O.94.a.14.i; O.94.a.14.v;
O.94.a.15.i; O.94.a.15.v; O.94.a.18.i; O.94.a.18.v; O.94.a.25.i; O.94.a.25.v;
O.94.e.4.i; O.94.e.4.v; O.94.e.6.i; O.94.e.6.v; O.94.e.11.i; O.94.e.11.v; O.94.e.14.i;
O.94.e.14.v; O.94.e.15.i; O.94.e.15.v; O.94.e.18.i; O.94.e.18.v; O.94.e.25.i;
O.94.e.25.v; O.94.g.4.i; O.94.g.4.v; O.94.g.6.i; O.94.g.6.v; O.94.g.11.i; O.94.g.11.v;
O.94.g.14.i; O.94.g.14.v; O.94.g.15.i; O.94.g.15.v; O.94.g.18.i; O.94.g.18.v;
O.94.g.25.i; O.94.g.25.v; O.94.l.4.i; O.94.l.4.v; O.94.l.6.i; O.94.l.6.v; O.94.l.11.i;
O.94.l.11.v; O.94.l.14.i; O.94.l.14.v; O.94.l.15.i; O.94.l.15.v; O.94.l.18.i; O.94.l.18.v;
O.94.l.25.i; O.94.l.25.v; O.94.m.4.i; O.94.m.4.v; O.94.m.6.i; O.94.m.6.v;
O.94.m.11.i; O.94.m.11.v; O.94.m.14.i; O.94.m.14.v; O.94.m.15.i; O.94.m.15.v;
O.94.m.18.i; O.94.m.18.v; O.94.m.25.i; O.94.m.25.v; O.94.o.4.i; O.94.o.4.v;
O.94.o.6.i; O.94.o.6.v; O.94.o.11.i; O.94.o.11.v; O.94.o.14.i; O.94.o.14.v;
O.94.o.15.i; O.94.o.15.v; O.94.o.18.i; O.94.o.18.v; O.94.o.25.i; O.94.o.25.v;
P.93.a.4.i; P.93.a.4.v; P.93.a.6.i; P.93.a.6.v; P.93.a.11.i; P.93.a.11.v; P.93.a.14.i;
P.93.a.14.v; P.93.a.15.i; P.93.a.15.v; P.93.a.18.i; P.93.a.18.v; P.93.a.25.i;
P.93.a.25.v; P.93.e.4.i; P.93.e.4.v; P.93.e.6.i; P.93.e.6.v; P.93.e.11.i; P.93.e.11.v;
P.93.e.14.i; P.93.e.14.v; P.93.e.15.i; P.93.e.15.v; P.93.e.18.i; P.93.e.18.v; P.93.e.25.i;
P.93.e.25.v; P.93.g.4.i; P.93.g.4.v; P.93.g.6.i; P.93.g.6.v; P.93.g.11.i; P.93.g.11.v;
P.93.g.14.i; P.93.g.14.v; P.93.g.15.i; P.93.g.15.v; P.93.g.18.i; P.93.g.18.v;
P.93.g.25.i; P.93.g.25.v; P.93.l.4.i; P.93.l.4.v; P.93.l.6.i; P.93.l.6.v; P.93.l.11.i;
P.93.l.11.v; P.93.l.14.i; P.93.l.14.v; P.93.l.15.i; P.93.l.15.v; P.93.l.18.i; P.93.l.18.v;
P.93.l.25.i; P.93.l.25.v; P.93.m.4.i; P.93.m.4.v; P.93.m.6.i; P.93.m.6.v; P.93.m.11.i;
P.93.m.11.v; P.93.m.14.i; P.93.m.14.v; P.93.m.15.i; P.93.m.15.v; P.93.m.18.i;
P.93.m.18.v; P.93.m.25.i; P.93.m.25.v; P.93.o.4.i; P.93.o.4.v; P.93.o.6.i; P.93.o.6.v;
P.93.o.11.i; P.93.o.11.v; P.93.o.14.i; P.93.o.14.v; P.93.o.15.i; P.93.o.15.v;
P.93.o.18.i; P.93.o.18.v; P.93.o.25.i; P.93.o.25.v; P.94.a.4.i; P.94.a.4.v; P.94.a.6.i;
P.94.a.6.v; P.94.a.11.i; P.94.a.11.v; P.94.a.14.i; P.94.a.14.v; P.94.a.15.i; P.94.a.15.v;
P.94.a.18.i; P.94.a.18.v; P.94.a.25.i; P.94.a.25.v; P.94.e.4.i; P.94.e.4.v; P.94.e.6.i;
P.94.e.6.v; P.94.e.11.i; P.94.e.11.v; P.94.e.14.i; P.94.e.14.v; P.94.e.15.i; P.94.e.15.v;
P.94.e.18.i; P.94.e.18.v; P.94.e.25.i; P.94.e.25.v; P.94.g.4.i; P.94.g.4.v; P.94.g.6.i;
P.94.g.6.v; P.94.g.11.i; P.94.g.11.v; P.94.g.14.i; P.94.g.14.v; P.94.g.15.i; P.94.g.15.v;
P.94.g.18.i; P.94.g.18.v; P.94.g.25.i; P.94.g.25.v; P.94.l.4.i; P.94.l.4.v; P.94.l.6.i;
P.94.l.6.v; P.94.l.11.i; P.94.l.11.v; P.94.l.14.i; P.94.l.14.v; P.94.l.15.i; P.94.l.15.v;
P.94.l.18.i; P.94.l.18.v; P.94.l.25.i; P.94.l.25.v; P.94.m.4.i; P.94.m.4.v; P.94.m.6.i;
P.94.m.6.v; P.94.m.11.i; P.94.m.11.v; P.94.m.14.i; P.94.m.14.v; P.94.m.15.i;
P.94.m.15.v; P.94.m.18.i; P.94.m.18.v; P.94.m.25.i; P.94.m.25.v; P.94.o.4.i;
P.94.o.4.v; P.94.o.6.i; P.94.o.6.v; P.94.o.11.i; P.94.o.11.v; P.94.o.14.i; P.94.o.14.v;
P.94.o.15.i; P.94.o.15.v; P.94.o.18.i; P.94.o.18.v; P.94.o.25.i; P.94.o.25.v; A.2.a.4.o;
A.2.a.4.bh; A.2.a.4.bi; A.2.a.4.bj; A.2.a.4.bk; A.2.a.11.o; A.2.a.11.bh; A.2.a.11.bi;
A.2.a.11.bj; A.2.a.11.bk; A.2.a.15.i; A.2.a.15.o; A.2.a.15.bh; A.2.a.15.bi;
A.2.a.15.bj; A.2.a.15.bk; A.2.a.37.i; A.2.a.37.o; A.2.a.37.bh; A.2.a.37.bi;
A.2.a.37.bj; A.2.a.37.bk; A.2.a.38.i; A.2.a.38.o; A.2.a.38.bh; A.2.a.38.bi;
A.2.a.38.bj; A.2.a.38.bk; A.2.a.39.i; A.2.a.39.o; A.2.a.39.bh; A.2.a.39.bi;
A.2.a.39.bj; A.2.a.39.bk; A.2.a.40.i; A.2.a.40.o; A.2.a.40.bh; A.2.a.40.bi;
A.2.a.40.bj; A.2.a.40.bk; A.2.a.41.i; A.2.a.41.o; A.2.a.41.bh; A.2.a.41.bi;
A.2.a.41.bj; A.2.a.41.bk; A.2.a.42.i; A.2.a.42.o; A.2.a.42.bh; A.2.a.42.bi;
A.2.a.42.bj; A.2.a.42.bk; A.2.a.43.i; A.2.a.43.o; A.2.a.43.bh; A.2.a.43.bi;
A.2.a.43.bj; A.2.a.43.bk; A.3.a.4.o; A.3.a.4.bh; A.3.a.4.bi; A.3.a.4.bj; A.3.a.4.bk;
A.3.a.11.o; A.3.a.11.bh; A.3.a.11.bi; A.3.a.11.bj; A.3.a.11.bk; A.3.a.15.i; A.3.a.15.o;
A.3.a.15.bh; A.3.a.15.bi; A.3.a.15.bj; A.3.a.15.bk; A.3.a.37.i; A.3.a.37.o;
A.3.a.37.bh; A.3.a.37.bi; A.3.a.37.bj; A.3.a.37.bk; A.3.a.38.i; A.3.a.38.o;
A.3.a.38.bh; A.3.a.38.bi; A.3.a.38.bj; A.3.a.38.bk; A.3.a.39.i; A.3.a.39.o;
A.3.a.39.bh; A.3.a.39.bi; A.3.a.39.bj; A.3.a.39.bk; A.3.a.40.i; A.3.a.40.o;
A.3.a.40.bh; A.3.a.40.bi; A.3.a.40.bj; A.3.a.40.bk; A.3.a.41.i; A.3.a.41.o;
A.3.a.41.bh; A.3.a.41.bi; A.3.a.41.bj; A.3.a.41.bk; A.3.a.42.i; A.3.a.42.o;
A.3.a.42.bh; A.3.a.42.bi; A.3.a.42.bj; A.3.a.42.bk; A.3.a.43.i; A.3.a.43.o;
A.3.a.43.bh; A.3.a.43.bi; A.3.a.43.bj; A.3.a.43.bk; A.4.a.4.o; A.4.a.4.bh; A.4.a.4.bi;
A.4.a.4.bj; A.4.a.4.bk; A.4.a.11.o; A.4.a.11.bh; A.4.a.11.bi; A.4.a.11.bj;
A.4.a.11.bk; A.4.a.15.i; A.4.a.15.o; A.4.a.15.bh; A.4.a.15.bi; A.4.a.15.bj;
A.4.a.15.bk; A.4.a.37.i; A.4.a.37.o; A.4.a.37.bh; A.4.a.37.bi; A.4.a.37.bj;
A.4.a.37.bk; A.4.a.38.i; A.4.a.38.o; A.4.a.38.bh; A.4.a.38.bi; A.4.a.38.bj;
A.4.a.38.bk; A.4.a.39.i; A.4.a.39.o; A.4.a.39.bh; A.4.a.39.bi; A.4.a.39.bj;
A.4.a.39.bk; A.4.a.40.i; A.4.a.40.o; A.4.a.40.bh; A.4.a.40.bi; A.4.a.40.bj;
A.4.a.40.bk; A.4.a.41.i; A.4.a.41.o; A.4.a.41.bh; A.4.a.41.bi; A.4.a.41.bj;
A.4.a.41.bk; A.4.a.42.i; A.4.a.42.o; A.4.a.42.bh; A.4.a.42.bi; A.4.a.42.bj;
A.4.a.42.bk; A.4.a.43.i; A.4.a.43.o; A.4.a.43.bh; A.4.a.43.bi; A.4.a.43.bj;
A.4.a.43.bk; A.7.a.4.o; A.7.a.4.bh; A.7.a.4.bi; A.7.a.4.bj; A.7.a.4.bk; A.7.a.11.o;
A.7.a.11.bh; A.7.a.11.bi; A.7.a.11.bj; A.7.a.11.bk; A.7.a.15.i; A.7.a.15.o;
A.7.a.15.bh; A.7.a.15.bi; A.7.a.15.bj; A.7.a.15.bk; A.7.a.37.i; A.7.a.37.o;
A.7.a.37.bh; A.7.a.37.bi; A.7.a.37.bj; A.7.a.37.bk; A.7.a.38.i; A.7.a.38.o;
A.7.a.38.bh; A.7.a.38.bi; A.7.a.38.bj; A.7.a.38.bk; A.7.a.39.i; A.7.a.39.o;
A.7.a.39.bh; A.7.a.39.bi; A.7.a.39.bj; A.7.a.39.bk; A.7.a.40.i; A.7.a.40.o;
A.7.a.40.bh; A.7.a.40.bi; A.7.a.40.bj; A.7.a.40.bk; A.7.a.41.i; A.7.a.41.o;
A.7.a.41.bh; A.7.a.41.bi; A.7.a.41.bj; A.7.a.41.bk; A.7.a.42.i; A.7.a.42.o;
A.7.a.42.bh; A.7.a.42.bi; A.7.a.42.bj; A.7.a.42.bk; A.7.a.43.i; A.7.a.43.o;
A.7.a.43.bh; A.7.a.43.bi; A.7.a.43.bj; A.7.a.43.bk; A.17.a.4.i; A.17.a.4.o;
A.17.a.4.bh; A.17.a.4.bi; A.17.a.4.bj; A.17.a.4.bk; A.17.a.11.i; A.17.a.11.o;
A.17.a.11.bh; A.17.a.11.bi; A.17.a.11.bj; A.17.a.11.bk; A.17.a.15.i; A.17.a.15.o;
A.17.a.15.bh; A.17.a.15.bi; A.17.a.15.bj; A.17.a.15.bk; A.17.a.37.i; A.17.a.37.o;
A.17.a.37.bh; A.17.a.37.bi; A.17.a.37.bj; A.17.a.37.bk; A.17.a.38.i; A.17.a.38.o;
A.17.a.38.bh; A.17.a.38.bi; A.17.a.38.bj; A.17.a.38.bk; A.17.a.39.i; A.17.a.39.o;
A.17.a.39.bh; A.17.a.39.bi; A.17.a.39.bj; A.17.a.39.bk; A.17.a.40.i; A.17.a.40.o;
A.17.a.40.bh; A.17.a.40.bi; A.17.a.40.bj; A.17.a.40.bk; A.17.a.41.i; A.17.a.41.o;
A.17.a.41.bh; A.17.a.41.bi; A.17.a.41.bj; A.17.a.41.bk; A.17.a.42.i; A.17.a.42.o;
A.17.a.42.bh; A.17.a.42.bi; A.17.a.42.bj; A.17.a.42.bk; A.17.a.43.i; A.17.a.43.o;
A.17.a.43.bh; A.17.a.43.bi; A.17.a.43.bj; A.17.a.43.bk; A.18.a.4.i; A.18.a.4.o;
A.18.a.4.bh; A.18.a.4.bi; A.18.a.4.bj; A.18.a.4.bk; A.18.a.11.i; A.18.a.11.o;
A.18.a.11.bh; A.18.a.11.bi; A.18.a.11.bj; A.18.a.11.bk; A.18.a.15.i; A.18.a.15.o;
A.18.a.15.bh; A.18.a.15.bi; A.18.a.15.bj; A.18.a.15.bk; A.18.a.37.i; A.18.a.37.o;
A.18.a.37.bh; A.18.a.37.bi; A.18.a.37.bj; A.18.a.37.bk; A.18.a.38.i; A.18.a.38.o;
A.18.a.38.bh; A.18.a.38.bi; A.18.a.38.bj; A.18.a.38.bk; A.18.a.39.i; A.18.a.39.o;
A.18.a.39.bh; A.18.a.39.bi; A.18.a.39.bj; A.18.a.39.bk; A.18.a.40.i; A.18.a.40.o;
A.18.a.40.bh; A.18.a.40.bi; A.18.a.40.bj; A.18.a.40.bk; A.18.a.41.i; A.18.a.41.o;
A.18.a.41.bh; A.18.a.41.bi; A.18.a.41.bj; A.18.a.41.bk; A.18.a.42.i; A.18.a.42.o;
A.18.a.42.bh; A.18.a.42.bi; A.18.a.42.bj; A.18.a.42.bk; A.18.a.43.i; A.18.a.43.o;
A.18.a.43.bh; A.18.a.43.bi; A.18.a.43.bj; A.18.a.43.bk; A.19.a.4.i; A.19.a.4.o;
A.19.a.4.bh; A.19.a.4.bi; A.19.a.4.bj; A.19.a.4.bk; A.19.a.11.i; A.19.a.11.o;
A.19.a.11.bh; A.19.a.11.bi; A.19.a.11.bj; A.19.a.11.bk; A.19.a.15.i; A.19.a.15.o;
A.19.a.15.bh; A.19.a.15.bi; A.19.a.15.bj; A.19.a.15.bk; A.19.a.37.i; A.19.a.37.o;
A.19.a.37.bh; A.19.a.37.bi; A.19.a.37.bj; A.19.a.37.bk; A.19.a.38.i; A.19.a.38.o;
A.19.a.38.bh; A.19.a.38.bi; A.19.a.38.bj; A.19.a.38.bk; A.19.a.39.i; A.19.a.39.o;
A.19.a.39.bh; A.19.a.39.bi; A.19.a.39.bj; A.19.a.39.bk; A.19.a.40.i; A.19.a.40.o;
A.19.a.40.bh; A.19.a.40.bi; A.19.a.40.bj; A.19.a.40.bk; A.19.a.41.i; A.19.a.41.o;
A.19.a.41.bh; A.19.a.41.bi; A.19.a.41.bj; A.19.a.41.bk; A.19.a.42.i; A.19.a.42.o;
A.19.a.42.bh; A.19.a.42.bi; A.19.a.42.bj; A.19.a.42.bk; A.19.a.43.i; A.19.a.43.o;
A.19.a.43.bh; A.19.a.43.bi; A.19.a.43.bj; A.19.a.43.bk; A.97.a.4.i; A.97.a.4.o;
A.97.a.4.bh; A.97.a.4.bi; A.97.a.4.bj; A.97.a.4.bk; A.97.a.11.i; A.97.a.11.o;
A.97.a.11.bh; A.97.a.11.bi; A.97.a.11.bj; A.97.a.11.bk; A.97.a.15.i; A.97.a.15.o;
A.97.a.15.bh; A.97.a.15.bi; A.97.a.15.bj; A.97.a.15.bk; A.97.a.37.i; A.97.a.37.o;
A.97.a.37.bh; A.97.a.37.bi; A.97.a.37.bj; A.97.a.37.bk; A.97.a.38.i; A.97.a.38.o;
A.97.a.38.bh; A.97.a.38.bi; A.97.a.38.bj; A.97.a.38.bk; A.97.a.39.i; A.97.a.39.o;
A.97.a.39.bh; A.97.a.39.bi; A.97.a.39.bj; A.97.a.39.bk; A.97.a.40.i; A.97.a.40.o;
A.97.a.40.bh; A.97.a.40.bi; A.97.a.40.bj; A.97.a.40.bk; A.97.a.41.i; A.97.a.41.o;
A.97.a.41.bh; A.97.a.41.bi; A.97.a.41.bj; A.97.a.41.bk; A.97.a.42.i; A.97.a.42.o;
A.97.a.42.bh; A.97.a.42.bi; A.97.a.42.bj; A.97.a.42.bk; A.97.a.43.i; A.97.a.43.o;
A.97.a.43.bh; A.97.a.43.bi; A.97.a.43.bj; A.97.a.43.bk; A.98.a.4.i; A.98.a.4.o;
A.98.a.4.bh; A.98.a.4.bi; A.98.a.4.bj; A.98.a.4.bk; A.98.a.11.i; A.98.a.11.o;
A.98.a.11.bh; A.98.a.11.bi; A.98.a.11.bj; A.98.a.11.bk; A.98.a.15.i; A.98.a.15.o;
A.98.a.15.bh; A.98.a.15.bi; A.98.a.15.bj; A.98.a.15.bk; A.98.a.37.i; A.98.a.37.o;
A.98.a.37.bh; A.98.a.37.bi; A.98.a.37.bj; A.98.a.37.bk; A.98.a.38.i; A.98.a.38.o;
A.98.a.38.bh; A.98.a.38.bi; A.98.a.38.bj; A.98.a.38.bk; A.98.a.39.i; A.98.a.39.o;
A.98.a.39.bh; A.98.a.39.bi; A.98.a.39.bj; A.98.a.39.bk; A.98.a.40.i; A.98.a.40.o;
A.98.a.40.bh; A.98.a.40.bi; A.98.a.40.bj; A.98.a.40.bk; A.98.a.41.i; A.98.a.41.o;
A.98.a.41.bh; A.98.a.41.bi; A.98.a.41.bj; A.98.a.41.bk; A.98.a.42.i; A.98.a.42.o;
A.98.a.42.bh; A.98.a.42.bi; A.98.a.42.bj; A.98.a.42.bk; A.98.a.43.i; A.98.a.43.o;
A.98.a.43.bh; A.98.a.43.bi; A.98.a.43.bj; A.98.a.43.bk; A.2.a.4.i; A.3.a.4.i; A.4.a.4.i;
A.5.a.4.i; A.6.a.4.i; A.7.a.4.i; A.9.a.4.i; A.10.a.4.i; A.15.a.4.i; A.100.a.4.i;
A.101.a.4.i; A.102.a.4.i; A.103.a.4.i; A.104.a.4.i; A.105.a.4.i; A.106.a.4.i;
A.107.a.4.i; A.108.a.4.i; A.109.a.4.i; A.110.a.4.i; A.111.a.4.i; A.112.a.4.i;
A.113.a.4.i; A.114.a.4.i; A.115.a.4.i; A.116.a.4.i; A.117.a.4.i; A.118.a.4.i;
A.119.a.4.i; A.120.a.4.i; A.121.a.4.i; A.122.a.4.i; A.123.a.4.i; A.124.a.4.i;
A.125.a.4.i; A.126.a.4.i; A.127.a.4.i; A.128.a.4.i; A.129.a.4.i; A.130.a.4.i;
A.131.a.4.i; A.132.a.4.i; A.133.a.4.i; A.134.a.4.i; A.135.a.4.i; A.136.a.4.i;
A.137.a.4.i; A.138.a.4.i; A.139.a.4.i; A.140.a.4.i; A.141.a.4.i; A.142.a.4.i;
A.143.a.4.i; A.144.a.4.i; A.145.a.4.i; A.146.a.4.i; A.147.a.4.i; A.148.a.4.i;
A.149.a.4.i; A.150.a.4.i; A.151.a.4.i; A.152.a.4.i; A.153.a.4.i; A.154.a.4.i;
A.155.a.4.i; A.156.a.4.i; A.157.a.4.i; A.158.a.4.i; A.159.a.4.i; A.160.a.4.i;
A.161.a.4.i; A.162.a.4.i; A.163.a.4.i; A.164.a.4.i; A.165.a.4.i; A.166.a.4.i;
A.167.a.4.i; A.168.a.4.i; A.169.a.4.i; A.170.a.4.i; A.171.a.4.i; A.172.a.4.i;
A.173.a.4.i; A.174.a.4.i; A.175.a.4.i; A.176.a.4.i; A.177.a.4.i; A.178.a.4.i;
A.179.a.4.i; A.180.a.4.i; A.181.a.4.i; A.182.a.4.i; A.183.a.4.i; A.184.a.4.i;
A.185.a.4.i; A.186.a.4.i; A.187.a.4.i; A.188.a.4.i; A.189.a.4.i; A.190.a.4.i;
A.191.a.4.i; A.192.a.4.i; A.193.a.4.i; A.194.a.4.i; A.195.a.4.i; A.196.a.4.i;
A.197.a.4.i; A.198.a.4.i; A.199.a.4.i; A.200.a.4.i; A.201.a.4.i; A.202.a.4.i;
A.203.a.4.i; A.204.a.4.i; A.205.a.4.i; A.206.a.4.i; A.207.a.4.i; A.208.a.4.i;
A.209.a.4.i; A.210.a.4.i; A.211.a.4.i; A.212.a.4.i; A.213.a.4.i; A.214.a.4.i;
A.215.a.4.i; A.216.a.4.i; A.217.a.4.i; A.218.a.4.i; A.219.a.4.i; A.220.a.4.i;
A.221.a.4.i; A.222.a.4.i; A.223.a.4.i; A.224.a.4.i; A.225.a.4.i; A.226.a.4.i;
A.227.a.4.i; A.228.a.4.i; A.229.a.4.i; A.230.a.4.i; A.231.a.4.i; A.232.a.4.i;
A.233.a.4.i; A.234.a.4.i; A.235.a.4.i; A.236.a.4.i; A.237.a.4.i; A.238.a.4.i;
A.239.a.4.i; A.240.a.4.i; A.241.a.4.i; A.242.a.4.i; A.243.a.4.i; A.244.a.4.i;
A.245.a.4.i; A.246.a.4.i; A.247.a.4.i; A.248.a.4.i; A.249.a.4.i; A.250.a.4.i;
A.251.a.4.i; A.252.a.4.i; A.253.a.4.i; A.254.a.4.i; A.255.a.4.i; A.256.a.4.i;
A.257.a.4.i; A.258.a.4.i; A.259.a.4.i; A.260.a.4.i; A.261.a.4.i; A.262.a.4.i;
A.263.a.4.i; A.264.a.4.i; A.265.a.4.i; A.266.a.4.i; A.267.a.4.i; A.268.a.4.i;
A.269.a.4.i; A.270.a.4.i; A.271.a.4.i; A.272.a.4.i; A.273.a.4.i; A.274.a.4.i;
A.275.a.4.i; A.276.a.4.i; A.277.a.4.i; A.278.a.4.i; A.279.a.4.i; A.280.a.4.i;
A.281.a.4.i; A.282.a.4.i; A.283.a.4.i; A.284.a.4.i; A.285.a.4.i; A.286.a.4.i;
A.287.a.4.i; A.288.a.4.i; A.289.a.4.i; A.290.a.4.i; A.291.a.4.i; A.292.a.4.i;
A.293.a.4.i; A.294.a.4.i; A.295.a.4.i; A.296.a.4.i; A.297.a.4.i; A.298.a.4.i;
A.299.a.4.i; A.300.a.4.i; A.301.a.4.i; A.302.a.4.i; A.303.a.4.i; A.304.a.4.i;
A.305.a.4.i; A.306.a.4.i; A.307.a.4.i; A.308.a.4.i; A.309.a.4.i; A.310.a.4.i;
A.311.a.4.i; A.312.a.4.i; A.313.a.4.i; A.314.a.4.i; A.315.a.4.i; A.316.a.4.i;
A.317.a.4.i; A.318.a.4.i; A.319.a.4.i; A.320.a.4.i; A.321.a.4.i; A.323.a.4.i;
A.324.a.4.i; A.325.a.4.i; A.326.a.4.i; A.327.a.4.i; A.328.a.4.i; A.329.a.4.i;
A.330.a.4.i; A.331.a.4.i; A.332.a.4.i; A.333.a.4.i; A.334.a.4.i; A.335.a.4.i;
A.336.a.4.i; A.337.a.4.i; A.338.a.4.i; A.339.a.4.i; A.340.a.4.i; A.341.a.4.i;
A.342.a.4.i; A.343.a.4.i; A.344.a.4.i; A.345.a.4.i; A.346.a.4.i; A.347.a.4.i;
A.348.a.4.i; A.349.a.4.i; A.350.a.4.i; A.351.a.4.i; A.352.a.4.i; A.353.a.4.i;
A.354.a.4.i; A.355.a.4.i; A.356.a.4.i; A.357.a.4.i; A.358.a.4.i; A.359.a.4.i;
A.360.a.4.i; A.361.a.4.i; A.362.a.4.i; A.363.a.4.i; A.364.a.4.i; A.365.a.4.i;
A.366.a.4.i; A.367.a.4.i; A.368.a.4.i; A.369.a.4.i; A.370.a.4.i; A.371.a.4.i;
A.372.a.4.i; A.373.a.4.i; A.374.a.4.i; A.375.a.4.i; A.376.a.4.i; A.377.a.4.i;
A.378.a.4.i; A.379.a.4.i; A.380.a.4.i; A.381.a.4.i; A.382.a.4.i; A.383.a.4.i;
A.384.a.4.i; A.385.a.4.i; A.386.a.4.i; A.387.a.4.i; A.388.a.4.i; A.389.a.4.i;
A.390.a.4.i; A.391.a.4.i; A.392.a.4.i; A.393.a.4.i; A.394.a.4.i; A.395.a.4.i;
A.396.a.4.i; A.397.a.4.i; A.398.a.4.i; A.399.a.4.i; A.400.a.4.i; A.401.a.4.i;
A.402.a.4.i; A.403.a.4.i; A.404.a.4.i; A.405.a.4.i; A.406.a.4.i; A.407.a.4.i;
A.408.a.4.i; A.409.a.4.i; A.410.a.4.i; A.411.a.4.i; A.412.a.4.i; A.413.a.4.i;
A.414.a.4.i; A.415.a.4.i; A.416.a.4.i; A.417.a.4.i; A.418.a.4.i; A.419.a.4.i;
A.420.a.4.i; A.421.a.4.i; A.422.a.4.i; A.423.a.4.i; A.424.a.4.i; A.425.a.4.i;
A.426.a.4.i; A.427.a.4.i; A.428.a.4.i; A.429.a.4.i; A.430.a.4.i; A.431.a.4.i;
A.432.a.4.i; A.433.a.4.i; A.434.a.4.i; A.435.a.4.i; A.436.a.4.i; A.437.a.4.i;
A.438.a.4.i; A.439.a.4.i; A.440.a.4.i; A.441.a.4.i; A.442.a.4.i; A.443.a.4.i;
A.444.a.4.i; A.445.a.4.i; A.446.a.4.i; A.447.a.4.i; A.448.a.4.i; A.449.a.4.i;
A.450.a.4.i; A.451.a.4.i; A.452.a.4.i; A.453.a.4.i; A.454.a.4.i; A.455.a.4.i;
A.456.a.4.i; A.457.a.4.i; A.458.a.4.i; A.459.a.4.i; A.460.a.4.i; A.461.a.4.i;
A.462.a.4.i; A.463.a.4.i; A.464.a.4.i; A.465.a.4.i; A.466.a.4.i; A.467.a.4.i;
A.468.a.4.i; A.469.a.4.i; A.470.a.4.i; A.471.a.4.i; A.472.a.4.i; A.473.a.4.i;
A.474.a.4.i; A.475.a.4.i; A.476.a.4.i; A.477.a.4.i; A.478.a.4.i; A.479.a.4.i;
A.480.a.4.i; A.481.a.4.i; A.482.a.4.i; A.483.a.4.i; A.484.a.4.i; A.485.a.4.i;
A.486.a.4.i; A.487.a.4.i; A.488.a.4.i; A.489.a.4.i; A.490.a.4.i; A.491.a.4.i;
A.492.a.4.i; A.493.a.4.i; A.494.a.4.i; A.495.a.4.i; A.496.a.4.i; A.497.a.4.i;
A.498.a.4.i; A.499.a.4.i; A.500.a.4.i; A.501.a.4.i; A.502.a.4.i; A.503.a.4.i;
A.504.a.4.i; A.505.a.4.i; A.506.a.4.i; A.507.a.4.i; A.508.a.4.i; A.509.a.4.i;
A.510.a.4.i; A.511.a.4.i; A.512.a.4.i; A.512.a.4.i; A.513.a.4.i; A.514.a.4.i;
A.515.a.4.i; A.516.a.4.i; A.517.a.4.i; A.518.a.4.i; A.519.a.4.i; A.520.a.4.i;
A.521.a.4.i; A.522.a.4.i; A.523.a.4.i; A.524.a.4.i; A.525.a.4.i; A.526.a.4.i;
A.527.a.4.i; A.528.a.4.i; A.529.a.4.i; A.530.a.4.i; A.531.a.4.i; A.532.a.4.i;
A.533.a.4.i; A.534.a.4.i; A.535.a.4.i; A.536.a.4.i; A.537.a.4.i; A.538.a.4.i;
A.539.a.4.i; A.540.a.4.i; A.541.a.4.i; A.542.a.4.i; A.543.a.4.i; A.544.a.4.i;
A.545.a.4.i; A.546.a.4.i; A.547.a.4.i; A.548.a.4.i; A.549.a.4.i; A.550.a.4.i;
A.551.a.4.i; A.552.a.4.i; A.553.a.4.i; A.554.a.4.i; A.555.a.4.i; A.556.a.4.i;
A.557.a.4.i; A.558.a.4.i; A.559.a.4.i; A.560.a.4.i; A.561.a.4.i; A.562.a.4.i;
A.563.a.4.i; A.564.a.4.i; A.565.a.4.i; A.566.a.4.i; A.567.a.4.i; A.568.a.4.i;
A.569.a.4.i; A.570.a.4.i; A.571.a.4.i; A.572.a.4.i; A.573.a.4.i; A.574.a.4.i;
A.575.a.4.i; A.576.a.4.i; A.577.a.4.i; A.578.a.4.i; A.579.a.4.i; A.580.a.4.i;
A.581.a.4.i; A.582.a.4.i; A.583.a.4.i; A.584.a.4.i; A.585.a.4.i; A.586.a.4.i;
A.587.a.4.i; A.588.a.4.i; A.589.a.4.i; A.590.a.4.i; A.591.a.4.i; A.592.a.4.i;
A.593.a.4.i; A.594.a.4.i; A.595.a.4.i; A.596.a.4.i; A.597.a.4.i; A.598.a.4.i;
A.599.a.4.i; A.600.a.4.i; A.601.a.4.i; A.602.a.4.i; A.603.a.4.i; A.604.a.4.i;
A.605.a.4.i; A.606.a.4.i; A.607.a.4.i; A.608.a.4.i; A.609.a.4.i; A.610.a.4.i;
A.611.a.4.i; A.612.a.4.i; A.613.a.4.i; A.614.a.4.i; A.615.a.4.i; A.616.a.4.i;
A.617.a.4.i; A.618.a.4.i; A.619.a.4.i; A.620.a.4.i; A.621.a.4.i; A.622.a.4.i;
A.623.a.4.i; A.624.a.4.i; A.625.a.4.i; A.626.a.4.i; A.627.a.4.i; A.628.a.4.i;
A.629.a.4.i; A.630.a.4.i; A.631.a.4.i; A.632.a.4.i; A.633.a.4.i; A.634.a.4.i;
A.635.a.4.i; A.636.a.4.i; A.637.a.4.i; A.638.a.4.i; A.639.a.4.i; A.640.a.4.i;
A.641.a.4.i; A.642.a.4.i; A.643.a.4.i; A.644.a.4.i; A.645.a.4.i; A.646.a.4.i;
A.647.a.4.i; A.648.a.4.i; A.649.a.4.i; A.650.a.4.i; A.651.a.4.i; A.652.a.4.i;
A.653.a.4.i; A.654.a.4.i; A.655.a.4.i; A.656.a.4.i; A.657.a.4.i; A.658.a.4.i;
A.659.a.4.i; A.660.a.4.i; A.2.a.11.i; A.3.a.11.i; A.4.a.11.i; A.5.a.11.i; A.6.a.11.i;
A.7.a.11.i; A.9.a.11.i; A.10.a.11.i; A.15.a.11.i; A.100.a.11.i; A.101.a.11.i;
A.102.a.11.i; A.103.a.11.i; A.104.a.11.i; A.105.a.11.i; A.106.a.11.i; A.107.a.11.i;
A.108.a.11.i; A.109.a.11.i; A.110.a.11.i; A.111.a.11.i; A.112.a.11.i; A.113.a.11.i;
A.114.a.11.i; A.115.a.11.i; A.116.a.11.i; A.117.a.11.i; A.118.a.11.i; A.119.a.11.i;
A.120.a.11.i; A.121.a.11.i; A.122.a.11.i; A.123.a.11.i; A.124.a.11.i; A.125.a.11.i;
A.126.a.11.i; A.127.a.11.i; A.128.a.11.i; A.129.a.11.i; A.130.a.11.i; A.131.a.11.i;
A.132.a.11.i; A.133.a.11.i; A.134.a.11.i; A.135.a.11.i; A.136.a.11.i; A.137.a.11.i;
A.138.a.11.i; A.139.a.11.i; A.140.a.11.i; A.141.a.11.i; A.142.a.11.i; A.143.a.11.i;
A.144.a.11.i; A.145.a.11.i; A.146.a.11.i; A.147.a.11.i; A.148.a.11.i; A.149.a.11.i;
A.150.a.11.i; A.151.a.11.i; A.152.a.11.i; A.153.a.11.i; A.154.a.11.i; A.155.a.11.i;
A.156.a.11.i; A.157.a.11.i; A.158.a.11.i; A.159.a.11.i; A.160.a.11.i; A.161.a.11.i;
A.162.a.11.i; A.163.a.11.i; A.164.a.11.i; A.165.a.11.i; A.166.a.11.i; A.167.a.11.i;
A.168.a.11.i; A.169.a.11.i; A.170.a.11.i; A.171.a.11.i; A.172.a.11.i; A.173.a.11.i;
A.174.a.11.i; A.175.a.11.i; A.176.a.11.i; A.177.a.11.i; A.178.a.11.i; A.179.a.11.i;
A.180.a.11.i; A.181.a.11.i; A.182.a.11.i; A.183.a.11.i; A.184.a.11.i; A.185.a.11.i;
A.186.a.11.i; A.187.a.11.i; A.188.a.11.i; A.189.a.11.i; A.190.a.11.i; A.191.a.11.i;
A.192.a.11.i; A.193.a.11.i; A.194.a.11.i; A.195.a.11.i; A.196.a.11.i; A.197.a.11.i;
A.198.a.11.i; A.199.a.11.i; A.200.a.11.i; A.201.a.11.i; A.202.a.11.i; A.203.a.11.i;
A.204.a.11.i; A.205.a.11.i; A.206.a.11.i; A.207.a.11.i; A.208.a.11.i; A.209.a.11.i;
A.210.a.11.i; A.211.a.11.i; A.212.a.11.i; A.213.a.11.i; A.214.a.11.i; A.215.a.11.i;
A.216.a.11.i; A.217.a.11.i; A.218.a.11.i; A.219.a.11.i; A.220.a.11.i; A.221.a.11.i;
A.222.a.11.i; A.223.a.11.i; A.224.a.11.i; A.225.a.11.i; A.226.a.11.i; A.227.a.11.i;
A.228.a.11.i; A.229.a.11.i; A.230.a.11.i; A.231.a.11.i; A.232.a.11.i; A.233.a.11.i;
A.234.a.11.i; A.235.a.11.i; A.236.a.11.i; A.237.a.11.i; A.238.a.11.i; A.239.a.11.i;
A.240.a.11.i; A.241.a.11.i; A.242.a.11.i; A.243.a.11.i; A.244.a.11.i; A.245.a.11.i;
A.246.a.11.i; A.247.a.11.i; A.248.a.11.i; A.249.a.11.i; A.250.a.11.i; A.251.a.11.i;
A.252.a.11.i; A.253.a.11.i; A.254.a.11.i; A.255.a.11.i; A.256.a.11.i; A.257.a.11.i;
A.258.a.11.i; A.259.a.11.i; A.260.a.11.i; A.261.a.11.i; A.262.a.11.i; A.263.a.11.i;
A.264.a.11.i; A.265.a.11.i; A.266.a.11.i; A.267.a.11.i; A.268.a.11.i; A.269.a.11.i;
A.270.a.11.i; A.271.a.11.i; A.272.a.11.i; A.273.a.11.i; A.274.a.11.i; A.275.a.11.i;
A.276.a.11.i; A.277.a.11.i; A.278.a.11.i; A.279.a.11.i; A.280.a.11.i; A.281.a.11.i;
A.282.a.11.i; A.283.a.11.i; A.284.a.11.i; A.285.a.11.i; A.286.a.11.i; A.287.a.11.i;
A.288.a.11.i; A.289.a.11.i; A.290.a.11.i; A.291.a.11.i; A.292.a.11.i; A.293.a.11.i;
A.294.a.11.i; A.295.a.11.i; A.296.a.11.i; A.297.a.11.i; A.298.a.11.i; A.299.a.11.i;
A.300.a.11.i; A.301.a.11.i; A.302.a.11.i; A.303.a.11.i; A.304.a.11.i; A.305.a.11.i;
A.306.a.11.i; A.307.a.11.i; A.308.a.11.i; A.309.a.11.i; A.310.a.11.i; A.311.a.11.i;
A.312.a.11.i; A.313.a.11.i; A.314.a.11.i; A.315.a.11.i; A.316.a.11.i; A.317.a.11.i;
A.318.a.11.i; A.319.a.11.i; A.320.a.11.i; A.321.a.11.i; A.323.a.11.i; A.324.a.11.i;
A.325.a.11.i; A.326.a.11.i; A.327.a.11.i; A.328.a.11.i; A.329.a.11.i; A.330.a.11.i;
A.331.a.11.i; A.332.a.11.i; A.333.a.11.i; A.334.a.11.i; A.335.a.11.i; A.336.a.11.i;
A.337.a.11.i; A.338.a.11.i; A.339.a.11.i; A.340.a.11.i; A.341.a.11.i; A.342.a.11.i;
A.343.a.11.i; A.344.a.11.i; A.345.a.11.i; A.346.a.11.i; A.347.a.11.i; A.348.a.11.i;
A.349.a.11.i; A.350.a.11.i; A.351.a.11.i; A.352.a.11.i; A.353.a.11.i; A.354.a.11.i;
A.355.a.11.i; A.356.a.11.i; A.357.a.11.i; A.358.a.11.i; A.359.a.11.i; A.360.a.11.i;
A.361.a.11.i; A.362.a.11.i; A.363.a.11.i; A.364.a.11.i; A.365.a.11.i; A.366.a.11.i;
A.367.a.11.i; A.368.a.11.i; A.369.a.11.i; A.370.a.11.i; A.371.a.11.i; A.372.a.11.i;
A.373.a.11.i; A.374.a.11.i; A.375.a.11.i; A.376.a.11.i; A.377.a.11.i; A.378.a.11.i;
A.379.a.11.i; A.380.a.11.i; A.381.a.11.i; A.382.a.11.i; A.383.a.11.i; A.384.a.11.i;
A.385.a.11.i; A.386.a.11.i; A.387.a.11.i; A.388.a.11.i; A.389.a.11.i; A.390.a.11.i;
A.391.a.11.i; A.392.a.11.i; A.393.a.11.i; A.394.a.11.i; A.395.a.11.i; A.396.a.11.i;
A.397.a.11.i; A.398.a.11.i; A.399.a.11.i; A.400.a.11.i; A.401.a.11.i; A.402.a.11.i;
A.403.a.11.i; A.404.a.11.i; A.405.a.11.i; A.406.a.11.i; A.407.a.11.i; A.408.a.11.i;
A.409.a.11.i; A.410.a.11.i; A.411.a.11.i; A.412.a.11.i; A.413.a.11.i; A.414.a.11.i;
A.415.a.11.i; A.416.a.11.i; A.417.a.11.i; A.418.a.11.i; A.419.a.11.i; A.420.a.11.i;
A.421.a.11.i; A.422.a.11.i; A.423.a.11.i; A.424.a.11.i; A.425.a.11.i; A.426.a.11.i;
A.427.a.11.i; A.428.a.11.i; A.429.a.11.i; A.430.a.11.i; A.431.a.11.i; A.432.a.11.i;
A.433.a.11.i; A.434.a.11.i; A.435.a.11.i; A.436.a.11.i; A.437.a.11.i; A.438.a.11.i;
A.439.a.11.i; A.440.a.11.i; A.441.a.11.i; A.442.a.11.i; A.443.a.11.i; A.444.a.11.i;
A.445.a.11.i; A.446.a.11.i; A.447.a.11.i; A.448.a.11.i; A.449.a.11.i; A.450.a.11.i;
A.451.a.11.i; A.452.a.11.i; A.453.a.11.i; A.454.a.11.i; A.455.a.11.i; A.456.a.11.i;
A.457.a.11.i; A.458.a.11.i; A.459.a.11.i; A.460.a.11.i; A.461.a.11.i; A.462.a.11.i;
A.463.a.11.i; A.464.a.11.i; A.465.a.11.i; A.466.a.11.i; A.467.a.11.i; A.468.a.11.i;
A.469.a.11.i; A.470.a.11.i; A.471.a.11.i; A.472.a.11.i; A.473.a.11.i; A.474.a.11.i;
A.475.a.11.i; A.476.a.11.i; A.477.a.11.i; A.478.a.11.i; A.479.a.11.i; A.480.a.11.i;
A.481.a.11.i; A.482.a.11.i; A.483.a.11.i; A.484.a.11.i; A.485.a.11.i; A.486.a.11.i;
A.487.a.11.i; A.488.a.11.i; A.489.a.11.i; A.490.a.11.i; A.491.a.11.i; A.492.a.11.i;
A.493.a.11.i; A.494.a.11.i; A.495.a.11.i; A.496.a.11.i; A.497.a.11.i; A.498.a.11.i;
A.499.a.11.i; A.500.a.11.i; A.501.a.11.i; A.502.a.11.i; A.503.a.11.i; A.504.a.11.i;
A.505.a.11.i; A.506.a.11.i; A.507.a.11.i; A.508.a.11.i; A.509.a.11.i; A.510.a.11.i;
A.511.a.11.i; A.512.a.11.i; A.512.a.11.i; A.513.a.11.i; A.514.a.11.i; A.515.a.11.i;
A.516.a.11.i; A.517.a.11.i; A.518.a.11.i; A.519.a.11.i; A.520.a.11.i; A.521.a.11.i;
A.522.a.11.i; A.523.a.11.i; A.524.a.11.i; A.525.a.11.i; A.526.a.11.i; A.527.a.11.i;
A.528.a.11.i; A.529.a.11.i; A.530.a.11.i; A.531.a.11.i; A.532.a.11.i; A.533.a.11.i;
A.534.a.11.i; A.535.a.11.i; A.536.a.11.i; A.537.a.11.i; A.538.a.11.i; A.539.a.11.i;
A.540.a.11.i; A.541.a.11.i; A.542.a.11.i; A.543.a.11.i; A.544.a.11.i; A.545.a.11.i;
A.546.a.11.i; A.547.a.11.i; A.548.a.11.i; A.549.a.11.i; A.550.a.11.i; A.551.a.11.i;
A.552.a.11.i; A.553.a.11.i; A.554.a.11.i; A.555.a.11.i; A.556.a.11.i; A.557.a.11.i;
A.558.a.11.i; A.559.a.11.i; A.560.a.11.i; A.561.a.11.i; A.562.a.11.i; A.563.a.11.i;
A.564.a.11.i; A.565.a.11.i; A.566.a.11.i; A.567.a.11.i; A.568.a.11.i; A.569.a.11.i;
A.570.a.11.i; A.571.a.11.i; A.572.a.11.i; A.573.a.11.i; A.574.a.11.i; A.575.a.11.i;
A.576.a.11.i; A.577.a.11.i; A.578.a.11.i; A.579.a.11.i; A.580.a.11.i; A.581.a.11.i;
A.582.a.11.i; A.583.a.11.i; A.584.a.11.i; A.585.a.11.i; A.586.a.11.i; A.587.a.11.i;
A.588.a.11.i; A.589.a.11.i; A.590.a.11.i; A.591.a.11.i; A.592.a.11.i; A.593.a.11.i;
A.594.a.11.i; A.595.a.11.i; A.596.a.11.i; A.597.a.11.i; A.598.a.11.i; A.599.a.11.i;
A.600.a.11.i; A.601.a.11.i; A.602.a.11.i; A.603.a.11.i; A.604.a.11.i; A.605.a.11.i;
A.606.a.11.i; A.607.a.11.i; A.608.a.11.i; A.609.a.11.i; A.610.a.11.i; A.611.a.11.i;
A.612.a.11.i; A.613.a.11.i; A.614.a.11.i; A.615.a.11.i; A.616.a.11.i; A.617.a.11.i;
A.618.a.11.i; A.619.a.11.i; A.620.a.11.i; A.621.a.11.i; A.622.a.11.i; A.623.a.11.i;
A.624.a.11.i; A.625.a.11.i; A.626.a.11.i; A.627.a.11.i; A.628.a.11.i; A.629.a.11.i;
A.630.a.11.i; A.631.a.11.i; A.632.a.11.i; A.633.a.11.i; A.634.a.11.i; A.635.a.11.i;
A.636.a.11.i; A.637.a.11.i; A.638.a.11.i; A.639.a.11.i; A.640.a.11.i; A.641.a.11.i;
A.642.a.11.i; A.643.a.11.i; A.644.a.11.i; A.645.a.11.i; A.646.a.11.i; A.647.a.11.i;
A.648.a.11.i; A.649.a.11.i; A.650.a.11.i; A.651.a.11.i; A.652.a.11.i; A.653.a.11.i;
A.654.a.11.i; A.655.a.11.i; A.656.a.11.i; A.657.a.11.i; A.658.a.11.i; A.659.a.11.i;
A.660.a.11.i; A.2.b.4.i; A.3.b.4.i; A.4.b.4.i; A.5.b.4.i; A.6.b.4.i; A.7.b.4.i; A.9.b.4.i;
A.10.b.4.i; A.15.b.4.i; A.100.b.4.i; A.101.b.4.i; A.102.b.4.i; A.103.b.4.i; A.104.b.4.i;
A.105.b.4.i; A.106.b.4.i; A.107.b.4.i; A.108.b.4.i; A.109.b.4.i; A.110.b.4.i;
A.111.b.4.i; A.112.b.4.i; A.113.b.4.i; A.114.b.4.i; A.115.b.4.i; A.116.b.4.i;
A.117.b.4.i; A.118.b.4.i; A.119.b.4.i; A.120.b.4.i; A.121.b.4.i; A.122.b.4.i;
A.123.b.4.i; A.124.b.4.i; A.125.b.4.i; A.126.b.4.i; A.127.b.4.i; A.128.b.4.i;
A.129.b.4.i; A.130.b.4.i; A.131.b.4.i; A.132.b.4.i; A.133.b.4.i; A.134.b.4.i;
A.135.b.4.i; A.136.b.4.i; A.137.b.4.i; A.138.b.4.i; A.139.b.4.i; A.140.b.4.i;
A.141.b.4.i; A.142.b.4.i; A.143.b.4.i; A.144.b.4.i; A.145.b.4.i; A.146.b.4.i;
A.147.b.4.i; A.148.b.4.i; A.149.b.4.i; A.150.b.4.i; A.151.b.4.i; A.152.b.4.i;
A.153.b.4.i; A.154.b.4.i; A.155.b.4.i; A.156.b.4.i; A.157.b.4.i; A.158.b.4.i;
A.159.b.4.i; A.160.b.4.i; A.161.b.4.i; A.162.b.4.i; A.163.b.4.i; A.164.b.4.i;
A.165.b.4.i; A.166.b.4.i; A.167.b.4.i; A.168.b.4.i; A.169.b.4.i; A.170.b.4.i;
A.171.b.4.i; A.172.b.4.i; A.173.b.4.i; A.174.b.4.i; A.175.b.4.i; A.176.b.4.i;
A.177.b.4.i; A.178.b.4.i; A.179.b.4.i; A.180.b.4.i; A.181.b.4.i; A.182.b.4.i;
A.183.b.4.i; A.184.b.4.i; A.185.b.4.i; A.186.b.4.i; A.187.b.4.i; A.188.b.4.i;
A.189.b.4.i; A.190.b.4.i; A.191.b.4.i; A.192.b.4.i; A.193.b.4.i; A.194.b.4.i;
A.195.b.4.i; A.196.b.4.i; A.197.b.4.i; A.198.b.4.i; A.199.b.4.i; A.200.b.4.i;
A.201.b.4.i; A.202.b.4.i; A.203.b.4.i; A.204.b.4.i; A.205.b.4.i; A.206.b.4.i;
A.207.b.4.i; A.208.b.4.i; A.209.b.4.i; A.210.b.4.i; A.211.b.4.i; A.212.b.4.i;
A.213.b.4.i; A.214.b.4.i; A.215.b.4.i; A.216.b.4.i; A.217.b.4.i; A.218.b.4.i;
A.219.b.4.i; A.220.b.4.i; A.221.b.4.i; A.222.b.4.i; A.223.b.4.i; A.224.b.4.i;
A.225.b.4.i; A.226.b.4.i; A.227.b.4.i; A.228.b.4.i; A.229.b.4.i; A.230.b.4.i;
A.231.b.4.i; A.232.b.4.i; A.233.b.4.i; A.234.b.4.i; A.235.b.4.i; A.236.b.4.i;
A.237.b.4.i; A.238.b.4.i; A.239.b.4.i; A.240.b.4.i; A.241.b.4.i; A.242.b.4.i;
A.243.b.4.i; A.244.b.4.i; A.245.b.4.i; A.246.b.4.i; A.247.b.4.i; A.248.b.4.i;
A.249.b.4.i; A.250.b.4.i; A.251.b.4.i; A.252.b.4.i; A.253.b.4.i; A.254.b.4.i;
A.255.b.4.i; A.256.b.4.i; A.257.b.4.i; A.258.b.4.i; A.259.b.4.i; A.260.b.4.i;
A.261.b.4.i; A.262.b.4.i; A.263.b.4.i; A.264.b.4.i; A.265.b.4.i; A.266.b.4.i;
A.267.b.4.i; A.268.b.4.i; A.269.b.4.i; A.270.b.4.i; A.271.b.4.i; A.272.b.4.i;
A.273.b.4.i; A.274.b.4.i; A.275.b.4.i; A.276.b.4.i; A.277.b.4.i; A.278.b.4.i;
A.279.b.4.i; A.280.b.4.i; A.281.b.4.i; A.282.b.4.i; A.283.b.4.i; A.284.b.4.i;
A.285.b.4.i; A.286.b.4.i; A.287.b.4.i; A.288.b.4.i; A.289.b.4.i; A.290.b.4.i;
A.291.b.4.i; A.292.b.4.i; A.293.b.4.i; A.294.b.4.i; A.295.b.4.i; A.296.b.4.i;
A.297.b.4.i; A.298.b.4.i; A.299.b.4.i; A.300.b.4.i; A.301.b.4.i; A.302.b.4.i;
A.303.b.4.i; A.304.b.4.i; A.305.b.4.i; A.306.b.4.i; A.307.b.4.i; A.308.b.4.i;
A.309.b.4.i; A.310.b.4.i; A.311.b.4.i; A.312.b.4.i; A.313.b.4.i; A.314.b.4.i;
A.315.b.4.i; A.316.b.4.i; A.317.b.4.i; A.318.b.4.i; A.319.b.4.i; A.320.b.4.i;
A.321.b.4.i; A.323.b.4.i; A.324.b.4.i; A.325.b.4.i; A.326.b.4.i; A.327.b.4.i;
A.328.b.4.i; A.329.b.4.i; A.330.b.4.i; A.331.b.4.i; A.332.b.4.i; A.333.b.4.i;
A.334.b.4.i; A.335.b.4.i; A.336.b.4.i; A.337.b.4.i; A.338.b.4.i; A.339.b.4.i;
A.340.b.4.i; A.341.b.4.i; A.342.b.4.i; A.343.b.4.i; A.344.b.4.i; A.345.b.4.i;
A.346.b.4.i; A.347.b.4.i; A.348.b.4.i; A.349.b.4.i; A.350.b.4.i; A.351.b.4.i;
A.352.b.4.i; A.353.b.4.i; A.354.b.4.i; A.355.b.4.i; A.356.b.4.i; A.357.b.4.i;
A.358.b.4.i; A.359.b.4.i; A.360.b.4.i; A.361.b.4.i; A.362.b.4.i; A.363.b.4.i;
A.364.b.4.i; A.365.b.4.i; A.366.b.4.i; A.367.b.4.i; A.368.b.4.i; A.369.b.4.i;
A.370.b.4.i; A.371.b.4.i; A.372.b.4.i; A.373.b.4.i; A.374.b.4.i; A.375.b.4.i;
A.376.b.4.i; A.377.b.4.i; A.378.b.4.i; A.379.b.4.i; A.380.b.4.i; A.381.b.4.i;
A.382.b.4.i; A.383.b.4.i; A.384.b.4.i; A.385.b.4.i; A.386.b.4.i; A.387.b.4.i;
A.388.b.4.i; A.389.b.4.i; A.390.b.4.i; A.391.b.4.i; A.392.b.4.i; A.393.b.4.i;
A.394.b.4.i; A.395.b.4.i; A.396.b.4.i; A.397.b.4.i; A.398.b.4.i; A.399.b.4.i;
A.400.b.4.i; A.401.b.4.i; A.402.b.4.i; A.403.b.4.i; A.404.b.4.i; A.405.b.4.i;
A.406.b.4.i; A.407.b.4.i; A.408.b.4.i; A.409.b.4.i; A.410.b.4.i; A.411.b.4.i;
A.412.b.4.i; A.413.b.4.i; A.414.b.4.i; A.415.b.4.i; A.416.b.4.i; A.417.b.4.i;
A.418.b.4.i; A.419.b.4.i; A.420.b.4.i; A.421.b.4.i; A.422.b.4.i; A.423.b.4.i;
A.424.b.4.i; A.425.b.4.i; A.426.b.4.i; A.427.b.4.i; A.428.b.4.i; A.429.b.4.i;
A.430.b.4.i; A.431.b.4.i; A.432.b.4.i; A.433.b.4.i; A.434.b.4.i; A.435.b.4.i;
A.436.b.4.i; A.437.b.4.i; A.438.b.4.i; A.439.b.4.i; A.440.b.4.i; A.441.b.4.i;
A.442.b.4.i; A.443.b.4.i; A.444.b.4.i; A.445.b.4.i; A.446.b.4.i; A.447.b.4.i;
A.448.b.4.i; A.449.b.4.i; A.450.b.4.i; A.451.b.4.i; A.452.b.4.i; A.453.b.4.i;
A.454.b.4.i; A.455.b.4.i; A.456.b.4.i; A.457.b.4.i; A.458.b.4.i; A.459.b.4.i;
A.460.b.4.i; A.461.b.4.i; A.462.b.4.i; A.463.b.4.i; A.464.b.4.i; A.465.b.4.i;
A.466.b.4.i; A.467.b.4.i; A.468.b.4d; A.469.b.4.i; A.470.b.4.i; A.471.b.4.i;
A.472.b.4.i; A.473.b.4.i; A.474.b.4.i; A.475.b.4.i; A.476.b.4.i; A.477.b.4.i;
A.478.b.4.i; A.479.b.4.i; A.480.b.4.i; A.481.b.4.i; A.482.b.4.i; A.483.b.4.i;
A.484.b.4.i; A.485.b.4.i; A.486.b.4.i; A.487.b.4.i; A.488.b.4.i; A.489.b.4.i;
A.490.b.4.i; A.491.b.4.i; A.492.b.4.i; A.493.b.4.i; A.494.b.4.i; A.495.b.4.i;
A.496.b.4.i; A.497.b.4.i; A.498.b.4.i; A.499.b.4.i; A.500.b.4.i; A.501.b.4.i;
A.502.b.4.i; A.503.b.4.i; A.504.b.4.i; A.505.b.4.i; A.506.b.4.i; A.507.b.4.i;
A.508.b.4.i; A.509.b.4.i; A.510.b.4.i; A.511.b.4.i; A.512.b.4.i; A.512.b.4.i;
A.513.b.4.i; A.514.b.4.i; A.515.b.4.i; A.516.b.4.i; A.517.b.4.i; A.518.b.4.i;
A.519.b.4.i; A.520.b.4.i; A.521.b.4.i; A.522.b.4.i; A.523.b.4.i; A.524.b.4.i;
A.525.b.4.i; A.526.b.4.i; A.527.b.4.i; A.528.b.4.i; A.529.b.4.i; A.530.b.4.i;
A.531.b.4.i; A.532.b.4.i; A.533.b.4.i; A.534.b.4.i; A.535.b.4.i; A.536.b.4.i;
A.537.b.4.i; A.538.b.4.i; A.539.b.4.i; A.540.b.4.i; A.541.b.4.i; A.542.b.4.i;
A.543.b.4.i; A.544.b.4.i; A.545.b.4.i; A.546.b.4.i; A.547.b.4.i; A.548.b.4.i;
A.549.b.4.i; A.550.b.4.i; A.551.b.4.i; A.552.b.4.i; A.553.b.4.i; A.554.b.4.i;
A.555.b.4.i; A.556.b.4.i; A.557.b.4.i; A.558.b.4.i; A.559.b.4.i; A.560.b.4.i;
A.561.b.4.i; A.562.b.4.i; A.563.b.4.i; A.564.b.4.i; A.565.b.4.i; A.566.b.4.i;
A.567.b.4.i; A.568.b.4.i; A.569.b.4.i; A.570.b.4.i; A.571.b.4.i; A.572.b.4.i;
A.573.b.4.i; A.574.b.4.i; A.575.b.4.i; A.576.b.4.i; A.577.b.4.i; A.578.b.4.i;
A.579.b.4.i; A.580.b.4.i; A.581.b.4.i; A.582.b.4.i; A.583.b.4.i; A.584.b.4.i;
A.585.b.4.i; A.586.b.4.i; A.587.b.4.i; A.588.b.4.i; A.589.b.4.i; A.590.b.4.i;
A.591.b.4.i; A.592.b.4.i; A.593.b.4.i; A.594.b.4.i; A.595.b.4.i; A.596.b.4.i;
A.597.b.4.i; A.598.b.4.i; A.599.b.4.i; A.600.b.4.i; A.601.b.4.i; A.602.b.4.i;
A.603.b.4.i; A.604.b.4.i; A.605.b.4.i; A.606.b.4.i; A.607.b.4.i; A.608.b.4.i;
A.609.b.4.i; A.610.b.4.i; A.611.b.4.i; A.612.b.4.i; A.613.b.4.i; A.614.b.4.i;
A.615.b.4.i; A.616.b.4.i; A.617.b.4.i; A.618.b.4.i; A.619.b.4.i; A.620.b.4.i;
A.621.b.4.i; A.622.b.4.i; A.623.b.4.i; A.624.b.4.i; A.625.b.4.i; A.626.b.4.i;
A.627.b.4.i; A.628.b.4.i; A.629.b.4.i; A.630.b.4.i; A.631.b.4.i; A.632.b.4.i;
A.633.b.4.i; A.634.b.4.i; A.635.b.4.i; A.636.b.4.i; A.637.b.4.i; A.638.b.4.i;
A.639.b.4.i; A.640.b.4.i; A.641.b.4.i; A.642.b.4.i; A.643.b.4.i; A.644.b.4.i;
A.645.b.4.i; A.646.b.4.i; A.647.b.4.i; A.648.b.4.i; A.649.b.4.i; A.650.b.4.i;
A.651.b.4.i; A.652.b.4.i; A.653.b.4.i; A.654.b.4.i; A.655.b.4.i; A.656.b.4.i;
A.657.b.4.i; A.658.b.4.i; A.659.b.4.i; A.660.b.4.i; A.2.b.11.i; A.3.b.11.i; A.4.b.11.i;
A.5.b.11.i; A.6.b.11.i; A.7.b.11.i; A.9.b.11.i; A.10.b.11.i; A.15.b.11.i; A.100.b.11.i;
A.101.b.11.i; A.102.b.11.i; A.103.b.11.i; A.104.b.11.i; A.105.b.11.i; A.106.b.11.i;
A.107.b.11.i; A.108.b.11.i; A.109.b.11.i; A.110.b.11.i; A.111.b.11.i; A.112.b.11.i;
A.113.b.11.i; A.114.b.11.i; A.115.b.11.i; A.116.b.11.i; A.117.b.11.i; A.118.b.11.i;
A.119.b.11.i; A.120.b.11.i; A.121.b.11.i; A.122.b.11.i; A.123.b.11.i; A.124.b.11.i;
A.125.b.11.i; A.126.b.11.i; A.127.b.11.i; A.128.b.11.i; A.129.b.11.i; A.130.b.11.i;
A.131.b.11.i; A.132.b.11.i; A.133.b.11.i; A.134.b.11.i; A.135.b.11.i; A.136.b.11.i;
A.137.b.11.i; A.138.b.11.i; A.139.b.11.i; A.140.b.11.i; A.141.b.11.i; A.142.b.11.i;
A.143.b.11.i; A.144.b.11.i; A.145.b.11.i; A.146.b.11.i; A.147.b.11.i; A.148.b.11.i;
A.149.b.11.i; A.150.b.11.i; A.151.b.11.i; A.152.b.11.i; A.153.b.11.i; A.154.b.11.i;
A.155.b.11.i; A.156.b.11.i; A.157.b.11.i; A.158.b.11.i; A.159.b.11.i; A.160.b.11.i;
A.161.b.11.i; A.162.b.11.i; A.163.b.11.i; A.164.b.11.i; A.165.b.11.i; A.166.b.11.i;
A.167.b.11.i; A.168.b.11.i; A.169.b.11.i; A.170.b.11.i; A.171.b.11.i; A.172.b.11.i;
A.173.b.11.i; A.174.b.11.i; A.175.b.11.i; A.176.b.11.i; A.177.b.11.i; A.178.b.11.i;
A.179.b.11.i; A.180.b.11.i; A.181.b.11.i; A.182.b.11.i; A.183.b.11.i; A.184.b.11.i;
A.185.b.11.i; A.186.b.11.i; A.187.b.11.i; A.188.b.11.i; A.189.b.11.i; A.190.b.11.i;
A.191.b.11.i; A.192.b.11.i; A.193.b.11.i; A.194.b.11.i; A.195.b.11.i; A.196.b.11.i;
A.197.b.11.i; A.198.b.11.i; A.199.b.11.i; A.200.b.11.i; A.201.b.11.i; A.202.b.11.i;
A.203.b.11.i; A.204.b.11.i; A.205.b.11.i; A.206.b.11.i; A.207.b.11.i; A.208.b.11.i;
A.209.b.11.i; A.210.b.11.i; A.211.b.11.i; A.212.b.11.i; A.213.b.11.i; A.214.b.11.i;
A.215.b.11.i; A.216.b.11.i; A.217.b.11.i; A.218.b.11.i; A.219.b.11.i; A.220.b.11.i;
A.221.b.11.i; A.222.b.11.i; A.223.b.11.i; A.224.b.11.i; A.225.b.11.i; A.226.b.11.i;
A.227.b.11.i; A.228.b.11.i; A.229.b.11.i; A.230.b.11.i; A.231.b.11.i; A.232.b.11.i;
A.233.b.11.i; A.234.b.11.i; A.235.b.11.i; A.236.b.11.i; A.237.b.11.i; A.238.b.11.i;
A.239.b.11.i; A.240.b.11.i; A.241.b.11.i; A.242.b.11.i; A.243.b.11.i; A.244.b.11.i;
A.245.b.11.i; A.246.b.11.i; A.247.b.11.i; A.248.b.11.i; A.249.b.11.i; A.250.b.11.i;
A.251.b.11.i; A.252.b.11.i; A.253.b.11.i; A.254.b.11.i; A.255.b.11.i; A.256.b.11.i;
A.257.b.11.i; A.258.b.11.i; A.259.b.11.i; A.260.b.11.i; A.261.b.11.i; A.262.b.11.i;
A.263.b.11.i; A.264.b.11.i; A.265.b.11.i; A.266.b.11.i; A.267.b.11.i; A.268.b.11.i;
A.269.b.11.i; A.270.b.11.i; A.271.b.11.i; A.272.b.11.i; A.273.b.11.i; A.274.b.11.i;
A.275.b.11.i; A.276.b.11.i; A.277.b.11.i; A.278.b.11.i; A.279.b.11.i; A.280.b.11.i;
A.281.b.11.i; A.282.b.11.i; A.283.b.11.i; A.284.b.11.i; A.285.b.11.i; A.286.b.11.i;
A.287.b.11.i; A.288.b.11.i; A.289.b.11.i; A.290.b.11.i; A.291.b.11.i; A.292.b.11.i;
A.293.b.11.i; A.294.b.11.i; A.295.b.11.i; A.296.b.11.i; A.297.b.11.i; A.298.b.11.i;
A.299.b.11.i; A.300.b.11.i; A.301.b.11.i; A.302.b.11.i; A.303.b.11.i; A.304.b.11.i;
A.305.b.11.i; A.306.b.11.i; A.307.b.11.i; A.308.b.11.i; A.309.b.11.i; A.310.b.11.i;
A.311.b.11.i; A.312.b.11.i; A.313.b.11.i; A.314.b.11.i; A.315.b.11.i; A.316.b.11.i;
A.317.b.11.i; A.318.b.11.i; A.319.b.11.i; A.320.b.11.i; A.321.b.11.i; A.323.b.11.i;
A.324.b.11.i; A.325.b.11.i; A.326.b.11.i; A.327.b.11.i; A.328.b.11.i; A.329.b.11.i;
A.330.b.11.i; A.331.b.11.i; A.332.b.11.i; A.333.b.11.i; A.334.b.11.i; A.335.b.11.i;
A.336.b.11.i; A.337.b.11.i; A.338.b.11.i; A.339.b.11.i; A.340.b.11.i; A.341.b.11.i;
A.342.b.11.i; A.343.b.11.i; A.344.b.11.i; A.345.b.11.i; A.346.b.11.i; A.347.b.11.i;
A.348.b.11.i; A.349.b.11.i; A.350.b.11.i; A.351.b.11.i; A.352.b.11.i; A.353.b.11.i;
A.354.b.11.i; A.355.b.11.i; A.356.b.11.i; A.357.b.11.i; A.358.b.11.i; A.359.b.11.i;
A.360.b.11.i; A.361.b.11.i; A.362.b.11.i; A.363.b.11.i; A.364.b.11.i; A.365.b.11.i;
A.366.b.11.i; A.367.b.11.i; A.368.b.11.i; A.369.b.11.i; A.370.b.11.i; A.371.b.11.i;
A.372.b.11.i; A.373.b.11.i; A.374.b.11.i; A.375.b.11.i; A.376.b.11.i; A.377.b.11.i;
A.378.b.11.i; A.379.b.11.i; A.380.b.11.i; A.381.b.11.i; A.382.b.11.i; A.383.b.11.i;
A.384.b.11.i; A.385.b.11.i; A.386.b.11.i; A.387.b.11.i; A.388.b.11.i; A.389.b.11.i;
A.390.b.11.i; A.391.b.11.i; A.392.b.11.i; A.393.b.11.i; A.394.b.11.i; A.395.b.11.i;
A.396.b.11.i; A.397.b.11.i; A.398.b.11.i; A.399.b.11.i; A.400.b.11.i; A.401.b.11.i;
A.402.b.11.i; A.403.b.11.i; A.404.b.11.i; A.405.b.11.i; A.406.b.11.i; A.407.b.11.i;
A.408.b.11.i; A.409.b.11.i; A.410.b.11.i; A.411.b.11.i; A.412.b.11.i; A.413.b.11.i;
A.414.b.11.i; A.415.b.11.i; A.416.b.11.i; A.417.b.11.i; A.418.b.11.i; A.419.b.11.i;
A.420.b.11.i; A.421.b.11.i; A.422.b.11.i; A.423.b.11.i; A.424.b.11.i; A.425.b.11.i;
A.426.b.11.i; A.427.b.11.i; A.428.b.11.i; A.429.b.11.i; A.430.b.11.i; A.431.b.11.i;
A.432.b.11.i; A.433.b.11.i; A.434.b.11.i; A.435.b.11.i; A.436.b.11.i; A.437.b.11.i;
A.438.b.11.i; A.439.b.11.i; A.440.b.11.i; A.441.b.11.i; A.442.b.11.i; A.443.b.11.i;
A.444.b.11.i; A.445.b.11.i; A.446.b.11.i; A.447.b.11.i; A.448.b.11.i; A.449.b.11.i;
A.450.b.11.i; A.451.b.11.i; A.452.b.11.i; A.453.b.11.i; A.454.b.11.i; A.455.b.11.i;
A.456.b.11.i; A.457.b.11.i; A.458.b.11.i; A.459.b.11.i; A.460.b.11.i; A.461.b.11.i;
A.462.b.11.i; A.463.b.11.i; A.464.b.11.i; A.465.b.11.i; A.466.b.11.i; A.467.b.11.i;
A.468.b.11.i; A.469.b.11.i; A.470.b.11.i; A.471.b.11.i; A.472.b.11.i; A.473.b.11.i;
A.474.b.11.i; A.475.b.11.i; A.476.b.11.i; A.477.b.11.i; A.478.b.11.i; A.479.b.11.i;
A.480.b.11.i; A.481.b.11.i; A.482.b.11.i; A.483.b.11.i; A.484.b.11.i; A.485.b.11.i;
A.486.b.11.i; A.487.b.11.i; A.488.b.11.i; A.489.b.11.i; A.490.b.11.i; A.491.b.11.i;
A.492.b.11.i; A.493.b.11.i; A.494.b.11.i; A.495.b.11.i; A.496.b.11.i; A.497.b.11.i;
A.498.b.11.i; A.499.b.11.i; A.500.b.11.i; A.501.b.11.i; A.502.b.11.i; A.503.b.11.i;
A.504.b.11.i; A.505.b.11.i; A.506.b.11.i; A.507.b.11.i; A.508.b.11.i; A.509.b.11.i;
A.510.b.11.i; A.511.b.11.i; A.512.b.11.i; A.512.b.11.i; A.513.b.11.i; A.514.b.11.i;
A.515.b.11.i; A.516.b.11.i; A.517.b.11.i; A.518.b.11.i; A.519.b.11.i; A.520.b.11.i;
A.521.b.11.i; A.522.b.11.i; A.523.b.11.i; A.524.b.11.i; A.525.b.11.i; A.526.b.11.i;
A.527.b.11.i; A.528.b.11.i; A.529.b.11.i; A.530.b.11.i; A.531.b.11.i; A.532.b.11.i;
A.533.b.11.i; A.534.b.11.i; A.535.b.11.i; A.536.b.11.i; A.537.b.11.i; A.538.b.11.i;
A.539.b.11.i; A.540.b.11.i; A.541.b.11.i; A.542.b.11.i; A.543.b.11.i; A.544.b.11.i;
A.545.b.11.i; A.546.b.11.i; A.547.b.11.i; A.548.b.11.i; A.549.b.11.i; A.550.b.11.i;
A.551.b.11.i; A.552.b.11.i; A.553.b.11.i; A.554.b.11.i; A.555.b.11.i; A.556.b.11.i;
A.557.b.11.i; A.558.b.11.i; A.559.b.11.i; A.560.b.11.i; A.561.b.11.i; A.562.b.11.i;
A.563.b.11.i; A.564.b.11.i; A.565.b.11.i; A.566.b.11.i; A.567.b.11.i; A.568.b.11.i;
A.569.b.11.i; A.570.b.11.i; A.571.b.11.i; A.572.b.11.i; A.573.b.11.i; A.574.b.11.i;
A.575.b.11.i; A.576.b.11.i; A.577.b.11.i; A.578.b.11.i; A.579.b.11.i; A.580.b.11.i;
A.581.b.11.i; A.582.b.11.i; A.583.b.11.i; A.584.b.11.i; A.585.b.11.i; A.586.b.11.i;
A.587.b.11.i; A.588.b.11.i; A.589.b.11.i; A.590.b.11.i; A.591.b.11.i; A.592.b.11.i;
A.593.b.11.i; A.594.b.11.i; A.595.b.11.i; A.596.b.11.i; A.597.b.11.i; A.598.b.11.i;
A.599.b.11.i; A.600.b.11.i; A.601.b.11.i; A.602.b.11.i; A.603.b.11.i; A.604.b.11.i;
A.605.b.11.i; A.606.b.11.i; A.607.b.11.i; A.608.b.11.i; A.609.b.11.i; A.610.b.11.i;
A.611.b.11.i; A.612.b.11.i; A.613.b.11.i; A.614.b.11.i; A.615.b.11.i; A.616.b.11.i;
A.617.b.11.i; A.618.b.11.i; A.619.b.11.i; A.620.b.11.i; A.621.b.11.i; A.622.b.11.i;
A.623.b.11.i; A.624.b.11.i; A.625.b.11.i; A.626.b.11.i; A.627.b.11.i; A.628.b.11.i;
A.629.b.11.i; A.630.b.11.i; A.631.b.11.i; A.632.b.11.i; A.633.b.11.i; A.634.b.11.i;
A.635.b.11.i; A.636.b.11.i; A.637.b.11.i; A.638.b.11.i; A.639.b.11.i; A.640.b.11.i;
A.641.b.11.i; A.642.b.11.i; A.643.b.11.i; A.644.b.11.i; A.645.b.11.i; A.646.b.11.i;
A.647.b.11.i; A.648.b.11.i; A.649.b.11.i; A.650.b.11.i; A.651.b.11.i; A.652.b.11.i;
A.653.b.11.i; A.654.b.11.i; A.655.b.11.i; A.656.b.11.i; A.657.b.11.i; A.658.b.11.i;
A.659.b.11.i; A.660.b.11.i; A.2.x.4.i; A.3.x.4.i; A.4.x.4.i; A.5.x.4.i; A.6.x.4.i;
A.7.x.4.i; A.9.x.4.i; A.10.x.4.i; A.15.x.4.i; A.100.x.4.i; A.101.x.4.i; A.102.x.4.i;
A.103.x.4.i; A.104.x.4.i; A.105.x.4.i; A.106.x.4.i; A.107.x.4.i; A.108.x.4.i;
A.109.x.4.i; A.110.x.4.i; A.111.x.4.i; A.112.x.4.i; A.113.x.4.i; A.114.x.4.i;
A.115.x.4.i; A.116.x.4.i; A.117.x.4.i; A.118.x.4.i; A.119.x.4.i; A.120.x.4.i;
A.121.x.4.i; A.122.x.4.i; A.123.x.4.i; A.124.x.4.i; A.125.x.4.i; A.126.x.4.i;
A.127.x.4.i; A.128.x.4.i; A.129.x.4.i; A.130.x.4.i; A.131.x.4.i; A.132.x.4.i;
A.133.x.4.i; A.134.x.4.i; A.135.x.4.i; A.136.x.4.i; A.137.x.4.i; A.138.x.4.i;
A.139.x.4.i; A.140.x.4.i; A.141.x.4.i; A.142.x.4.i; A.143.x.4.i; A.144.x.4.i;
A.145.x.4.i; A.146.x.4.i; A.147.x.4.i; A.148.x.4.i; A.149.x.4.i; A.150.x.4.i;
A.151.x.4.i; A.152.x.4.i; A.153.x.4.i; A.154.x.4.i; A.155.x.4.i; A.156.x.4.i;
A.157.x.4.i; A.158.x.4.i; A.159.x.4.i; A.160.x.4.i; A.161.x.4.i; A.162.x.4.i;
A.163.x.4.i; A.164.x.4.i; A.165.x.4.i; A.166.x.4.i; A.167.x.4.i; A.168.x.4.i;
A.169.x.4.i; A.170.x.4.i; A.171.x.4.i; A.172.x.4.i; A.173.x.4.i; A.174.x.4.i;
A.175.x.4.i; A.176.x.4.i; A.177.x.4.i; A.178.x.4.i; A.179.x.4.i; A.180.x.4.i;
A.181.x.4.i; A.182.x.4.i; A.183.x.4.i; A.184.x.4.i; A.185.x.4.i; A.186.x.4.i;
A.187.x.4.i; A.188.x.4.i; A.189.x.4.i; A.190.x.4.i; A.191.x.4.i; A.192.x.4.i;
A.193.x.4.i; A.194.x.4.i; A.195.x.4.i; A.196.x.4.i; A.197.x.4.i; A.198.x.4.i;
A.199.x.4.i; A.200.x.4.i; A.201.x.4.i; A.202.x.4.i; A.203.x.4.i; A.204.x.4.i;
A.205.x.4.i; A.206.x.4.i; A.207.x.4.i; A.208.x.4.i; A.209.x.4.i; A.210.x.4.i;
A.211.x.4.i; A.212.x.4.i; A.213.x.4.i; A.214.x.4.i; A.215.x.4.i; A.216.x.4.i;
A.217.x.4.i; A.218.x.4.i; A.219.x.4.i; A.220.x.4.i; A.221.x.4.i; A.222.x.4.i;
A.223.x.4.i; A.224.x.4.i; A.225.x.4.i; A.226.x.4.i; A.227.x.4.i; A.228.x.4.i;
A.229.x.4.i; A.230.x.4.i; A.231.x.4.i; A.232.x.4.i; A.233.x.4.i; A.234.x.4.i;
A.235.x.4.i; A.236.x.4.i; A.237.x.4.i; A.238.x.4.i; A.239.x.4.i; A.240.x.4.i;
A.241.x.4.i; A.242.x.4.i; A.243.x.4.i; A.244.x.4.i; A.245.x.4.i; A.246.x.4.i;
A.247.x.4.i; A.248.x.4.i; A.249.x.4.i; A.250.x.4.i; A.251.x.4.i; A.252.x.4.i;
A.253.x.4.i; A.254.x.4.i; A.255.x.4.i; A.256.x.4.i; A.257.x.4.i; A.258.x.4.i;
A.259.x.4.i; A.260.x.4.i; A.261.x.4.i; A.262.x.4.i; A.263.x.4.i; A.264.x.4.i;
A.265.x.4.i; A.266.x.4.i; A.267.x.4.i; A.268.x.4.i; A.269.x.4.i; A.270.x.4.i;
A.271.x.4.i; A.272.x.4.i; A.273.x.4.i; A.274.x.4.i; A.275.x.4.i; A.276.x.4.i;
A.277.x.4.i; A.278.x.4.i; A.279.x.4.i; A.280.x.4.i; A.281.x.4.i; A.282.x.4.i;
A.283.x.4.i; A.284.x.4.i; A.285.x.4.i; A.286.x.4.i; A.287.x.4.i; A.288.x.4.i;
A.289.x.4.i; A.290.x.4.i; A.291.x.4.i; A.292.x.4.i; A.293.x.4.i; A.294.x.4.i;
A.295.x.4.i; A.296.x.4.i; A.297.x.4.i; A.298.x.4.i; A.299.x.4.i; A.300.x.4.i;
A.301.x.4.i; A.302.x.4.i; A.303.x.4.i; A.304.x.4.i; A.305.x.4.i; A.306.x.4.i;
A.307.x.4.i; A.308.x.4.i; A.309.x.4.i; A.310.x.4.i; A.311.x.4.i; A.312.x.4.i;
A.313.x.4.i; A.314.x.4.i; A.315.x.4.i; A.316.x.4.i; A.317.x.4.i; A.318.x.4.i;
A.319.x.4.i; A.320.x.4.i; A.321.x.4.i; A.323.x.4.i; A.324.x.4.i; A.325.x.4.i;
A.326.x.4.i; A.327.x.4.i; A.328.x.4.i; A.329.x.4.i; A.330.x.4.i; A.331.x.4.i;
A.332.x.4.i; A.333.x.4.i; A.334.x.4.i; A.335.x.4.i; A.336.x.4.i; A.337.x.4.i;
A.338.x.4.i; A.339.x.4.i; A.340.x.4.i; A.341.x.4.i; A.342.x.4.i; A.343.x.4.i;
A.344.x.4.i; A.345.x.4.i; A.346.x.4.i; A.347.x.4.i; A.348.x.4.i; A.349.x.4.i;
A.350.x.4.i; A.351.x.4.i; A.352.x.4.i; A.353.x.4.i; A.354.x.4.i; A.355.x.4.i;
A.356.x.4.i; A.357.x.4.i; A.358.x.4.i; A.359.x.4.i; A.360.x.4.i; A.361.x.4.i;
A.362.x.4.i; A.363.x.4.i; A.364.x.4.i; A.365.x.4.i; A.366.x.4.i; A.367.x.4.i;
A.368.x.4.i; A.369.x.4.i; A.370.x.4.i; A.371.x.4.i; A.372.x.4.i; A.373.x.4.i;
A.374.x.4.i; A.375.x.4.i; A.376.x.4.i; A.377.x.4.i; A.378.x.4.i; A.379.x.4.i;
A.380.x.4.i; A.381.x.4.i; A.382.x.4.i; A.383.x.4.i; A.384.x.4.i; A.385.x.4.i;
A.386.x.4.i; A.387.x.4.i; A.388.x.4.i; A.389.x.4.i; A.390.x.4.i; A.391.x.4.i;
A.392.x.4.i; A.393.x.4.i; A.394.x.4.i; A.395.x.4.i; A.396.x.4.i; A.397.x.4.i;
A.398.x.4.i; A.399.x.4.i; A.400.x.4.i; A.401.x.4.i; A.402.x.4.i; A.403.x.4.i;
A.404.x.4.i; A.405.x.4.i; A.406.x.4.i; A.407.x.4.i; A.408.x.4.i; A.409.x.4.i;
A.410.x.4.i; A.411.x.4.i; A.412.x.4.i; A.413.x.4.i; A.414.x.4.i; A.415.x.4.i;
A.416.x.4.i; A.417.x.4.i; A.418.x.4.i; A.419.x.4.i; A.420.x.4.i; A.421.x.4.i;
A.422.x.4.i; A.423.x.4.i; A.424.x.4.i; A.425.x.4.i; A.426.x.4.i; A.427.x.4.i;
A.428.x.4.i; A.429.x.4.i; A.430.x.4.i; A.431.x.4.i; A.432.x.4.i; A.433.x.4.i;
A.434.x.4.i; A.435.x.4.i; A.436.x.4.i; A.437.x.4.i; A.438.x.4.i; A.439.x.4.i;
A.440.x.4.i; A.441.x.4.i; A.442.x.4.i; A.443.x.4.i; A.444.x.4.i; A.445.x.4.i;
A.446.x.4.i; A.447.x.4.i; A.448.x.4.i; A.449.x.4.i; A.450.x.4.i; A.451.x.4.i;
A.452.x.4.i; A.453.x.4.i; A.454.x.4.i; A.455.x.4.i; A.456.x.4.i; A.457.x.4.i;
A.458.x.4.i; A.459.x.4.i; A.460.x.4.i; A.461.x.4.i; A.462.x.4.i; A.463.x.4.i;
A.464.x.4.i; A.465.x.4.i; A.466.x.4.i; A.467.x.4.i; A.468.x.4.i; A.469.x.4.i;
A.470.x.4.i; A.471.x.4.i; A.472.x.4.i; A.473.x.4.i; A.474.x.4.i; A.475.x.4.i;
A.476.x.4.i; A.477.x.4.i; A.478.x.4.i; A.479.x.4.i; A.480.x.4.i; A.481.x.4.i;
A.482.x.4.i; A.483.x.4.i; A.484.x.4.i; A.485.x.4.i; A.486.x.4.i; A.487.x.4.i;
A.488.x.4.i; A.489.x.4.i; A.490.x.4.i; A.491.x.4.i; A.492.x.4.i; A.493.x.4.i;
A.494.x.4.i; A.495.x.4.i; A.496.x.4.i; A.497.x.4.i; A.498.x.4.i; A.499.x.4.i;
A.500.x.4.i; A.501.x.4.i; A.502.x.4.i; A.503.x.4.i; A.504.x.4.i; A.505.x.4.i;
A.506.x.4.i; A.507.x.4.i; A.508.x.4.i; A.509.x.4.i; A.510.x.4.i; A.511.x.4.i;
A.512.x.4.i; A.512.x.4.i; A.513.x.4.i; A.514.x.4.i; A.515.x.4.i; A.516.x.4.i;
A.517.x.4.i; A.518.x.4.i; A.519.x.4.i; A.520.x.4.i; A.521.x.4.i; A.522.x.4.i;
A.523.x.4.i; A.524.x.4.i; A.525.x.4.i; A.526.x.4.i; A.527.x.4.i; A.528.x.4.i;
A.529.x.4.i; A.530.x.4.i; A.531.x.4.i; A.532.x.4.i; A.533.x.4.i; A.534.x.4.i;
A.535.x.4.i; A.536.x.4.i; A.537.x.4.i; A.538.x.4.i; A.539.x.4.i; A.540.x.4.i;
A.541.x.4.i; A.542.x.4.i; A.543.x.4.i; A.544.x.4.i; A.545.x.4.i; A.546.x.4.i;
A.547.x.4.i; A.548.x.4.i; A.549.x.4.i; A.550.x.4.i; A.551.x.4.i; A.552.x.4.i;
A.553.x.4.i; A.554.x.4.i; A.555.x.4.i; A.556.x.4.i; A.557.x.4.i; A.558.x.4.i;
A.559.x.4.i; A.560.x.4.i; A.561.x.4.i; A.562.x.4.i; A.563.x.4.i; A.564.x.4.i;
A.565.x.4.i; A.566.x.4.i; A.567.x.4.i; A.568.x.4.i; A.569.x.4.i; A.570.x.4.i;
A.571.x.4.i; A.572.x.4.i; A.573.x.4.i; A.574.x.4.i; A.575.x.4.i; A.576.x.4.i;
A.577.x.4.i; A.578.x.4.i; A.579.x.4.i; A.580.x.4.i; A.581.x.4.i; A.582.x.4.i;
A.583.x.4.i; A.584.x.4.i; A.585.x.4.i; A.586.x.4.i; A.587.x.4.i; A.588.x.4.i;
A.589.x.4.i; A.590.x.4.i; A.591.x.4.i; A.592.x.4.i; A.593.x.4.i; A.594.x.4.i;
A.595.x.4.i; A.596.x.4.i; A.597.x.4.i; A.598.x.4.i; A.599.x.4.i; A.600.x.4.i;
A.601.x.4.i; A.602.x.4.i; A.603.x.4.i; A.604.x.4.i; A.605.x.4.i; A.606.x.4.i;
A.607.x.4.i; A.608.x.4.i; A.609.x.4.i; A.610.x.4.i; A.611.x.4.i; A.612.x.4.i;
A.613.x.4.i; A.614.x.4.i; A.615.x.4.i; A.616.x.4.i; A.617.x.4.i; A.618.x.4.i;
A.619.x.4.i; A.620.x.4.i; A.621.x.4.i; A.622.x.4.i; A.623.x.4.i; A.624.x.4.i;
A.625.x.4.i; A.626.x.4.i; A.627.x.4.i; A.628.x.4.i; A.629.x.4.i; A.630.x.4.i;
A.631.x.4.i; A.632.x.4.i; A.633.x.4.i; A.634.x.4.i; A.635.x.4.i; A.636.x.4.i;
A.637.x.4.i; A.638.x.4.i; A.639.x.4.i; A.640.x.4.i; A.641.x.4.i; A.642.x.4.i;
A.643.x.4.i; A.644.x.4.i; A.645.x.4.i; A.646.x.4.i; A.647.x.4.i; A.648.x.4.i;
A.649.x.4.i; A.650.x.4.i; A.651.x.4.i; A.652.x.4.i; A.653.x.4.i; A.654.x.4.i;
A.655.x.4.i; A.656.x.4.i; A.657.x.4.i; A.658.x.4.i; A.659.x.4.i; A.660.x.4.i; A.2.x.11.i;
A.3.x.11.i; A.4.x.11.i; A.5.x.11.i; A.6.x.11.i; A.7.x.11.i; A.9.x.11.i; A.10.x.11.i;
A.15.x.11.i; A.100.x.11.i; A.101.x.11.i; A.102.x.11.i; A.103.x.11.i; A.104.x.11.i;
A.105.x.11.i; A.106.x.11.i; A.107.x.11.i; A.108.x.11.i; A.109.x.11.i; A.110.x.11.i;
A.111.x.11.i; A.112.x.11.i; A.113.x.11.i; A.114.x.11.i; A.115.x.11.i; A.116.x.11.i;
A.117.x.11.i; A.118.x.11.i; A.119.x.11.i; A.120.x.11.i; A.121.x.11.i; A.122.x.11.i;
A.123.x.11.i; A.124.x.11.i; A.125.x.11.i; A.126.x.11.i; A.127.x.11.i; A.128.x.11.i;
A.129.x.11.i; A.130.x.11.i; A.131.x.11.i; A.132.x.11.i; A.133.x.11.i; A.134.x.11.i;
A.135.x.11.i; A.136.x.11.i; A.137.x.11.i; A.138.x.11.i; A.139.x.11.i; A.140.x.11.i;
A.141.x.11.i; A.142.x.11.i; A.143.x.11.i; A.144.x.11.i; A.145.x.11.i; A.146.x.11.i;
A.147.x.11.i; A.148.x.11.i; A.149.x.11.i; A.150.x.11.i; A.151.x.11.i; A.152.x.11.i;
A.153.x.11.i; A.154.x.11.i; A.155.x.11.i; A.156.x.11.i; A.157.x.11.i; A.158.x.11.i;
A.159.x.11.i; A.160.x.11.i; A.161.x.11.i; A.162.x.11.i; A.163.x.11.i; A.164.x.11.i;
A.165.x.11.i; A.166.x.11.i; A.167.x.11.i; A.168.x.11.i; A.169.x.11.i; A.170.x.11.i;
A.171.x.11.i; A.172.x.11.i; A.173.x.11.i; A.174.x.11.i; A.175.x.11.i; A.176.x.11.i;
A.177.x.11.i; A.178.x.11.i; A.179.x.11.i; A.180.x.11.i; A.181.x.11.i; A.182.x.11.i;
A.183.x.11.i; A.184.x.11.i; A.185.x.11.i; A.186.x.11.i; A.187.x.11.i; A.188.x.11.i;
A.189.x.11.i; A.190.x.11.i; A.191.x.11.i; A.192.x.11.i; A.193.x.11.i; A.194.x.11.i;
A.195.x.11.i; A.196.x.11.i; A.197.x.11.i; A.198.x.11.i; A.199.x.11.i; A.200.x.11.i;
A.201.x.11.i; A.202.x.11.i; A.203.x.11.i; A.204.x.11.i; A.205.x.11.i; A.206.x.11.i;
A.207.x.11.i; A.208.x.11.i; A.209.x.11.i; A.210.x.11.i; A.211.x.11.i; A.212.x.11.i;
A.213.x.11.i; A.214.x.11.i; A.215.x.11.i; A.216.x.11.i; A.217.x.11.i; A.218.x.11.i;
A.219.x.11.i; A.220.x.11.i; A.221.x.11.i; A.222.x.11.i; A.223.x.11.i; A.224.x.11.i;
A.225.x.11.i; A.226.x.11.i; A.227.x.11.i; A.228.x.11.i; A.229.x.11.i; A.230.x.11.i;
A.231.x.11.i; A.232.x.11.i; A.233.x.11.i; A.234.x.11.i; A.235.x.11.i; A.236.x.11.i;
A.237.x.11.i; A.238.x.11.i; A.239.x.11.i; A.240.x.11.i; A.241.x.11.i; A.242.x.11.i;
A.243.x.11.i; A.244.x.11.i; A.245.x.11.i; A.246.x.11.i; A.247.x.11.i; A.248.x.11.i;
A.249.x.11.i; A.250.x.11.i; A.251.x.11.i; A.252.x.11.i; A.253.x.11.i; A.254.x.11.i;
A.255.x.11.i; A.256.x.11.i; A.257.x.11.i; A.258.x.11.i; A.259.x.11.i; A.260.x.11.i;
A.261.x.11.i; A.262.x.11.i; A.263.x.11.i; A.264.x.11.i; A.265.x.11.i; A.266.x.11.i;
A.267.x.11.i; A.268.x.11.i; A.269.x.11.i; A.270.x.11.i; A.271.x.11.i; A.272.x.11.i;
A.273.x.11.i; A.274.x.11.i; A.275.x.11.i; A.276.x.11.i; A.277.x.11.i; A.278.x.11.i;
A.279.x.11.i; A.280.x.11.i; A.281.x.11.i; A.282.x.11.i; A.283.x.11.i; A.284.x.11.i;
A.285.x.11.i; A.286.x.11.i; A.287.x.11.i; A.288.x.11.i; A.289.x.11.i; A.290.x.11.i;
A.291.x.11.i; A.292.x.11.i; A.293.x.11.i; A.294.x.11.i; A.295.x.11.i; A.296.x.11.i;
A.297.x.11.i; A.298.x.11.i; A.299.x.11.i; A.300.x.11.i; A.301.x.11.i; A.302.x.11.i;
A.303.x.11.i; A.304.x.11.i; A.305.x.11.i; A.306.x.11.i; A.307.x.11.i; A.308.x.11.i;
A.309.x.11.i; A.310.x.11.i; A.311.x.11.i; A.312.x.11.i; A.313.x.11.i; A.314.x.11.i;
A.315.x.11.i; A.316.x.11.i; A.317.x.11.i; A.318.x.11.i; A.319.x.11.i; A.320.x.11.i;
A.321.x.11.i; A.323.x.11.i; A.324.x.11.i; A.325.x.11.i; A.326.x.11.i; A.327.x.11.i;
A.328.x.11.i; A.329.x.11.i; A.330.x.11.i; A.331.x.11.i; A.332.x.11.i; A.333.x.11.i;
A.334.x.11.i; A.335.x.11.i; A.336.x.11.i; A.337.x.11.i; A.338.x.11.i; A.339.x.11.i;
A.340.x.11.i; A.341.x.11.i; A.342.x.11.i; A.343.x.11.i; A.344.x.11.i; A.345.x.11.i;
A.346.x.11.i; A.347.x.11.i; A.348.x.11.i; A.349.x.11.i; A.350.x.11.i; A.351.x.11.i;
A.352.x.11.i; A.353.x.11.i; A.354.x.11.i; A.355.x.11.i; A.356.x.11.i; A.357.x.11.i;
A.358.x.11.i; A.359.x.11.i; A.360.x.11.i; A.361.x.11.i; A.362.x.11.i; A.363.x.11.i;
A.364.x.11.i; A.365.x.11.i; A.366.x.11.i; A.367.x.11.i; A.368.x.11.i; A.369.x.11.i;
A.370.x.11.i; A.371.x.11.i; A.372.x.11.i; A.373.x.11.i; A.374.x.11.i; A.375.x.11.i;
A.376.x.11.i; A.377.x.11.i; A.378.x.11.i; A.379.x.11.i; A.380.x.11.i; A.381.x.11.i;
A.382.x.11.i; A.383.x.11.i; A.384.x.11.i; A.385.x.11.i; A.386.x.11.i; A.387.x.11.i;
A.388.x.11.i; A.389.x.11.i; A.390.x.11.i; A.391.x.11.i; A.392.x.11.i; A.393.x.11.i;
A.394.x.11.i; A.395.x.11.i; A.396.x.11.i; A.397.x.11.i; A.398.x.11.i; A.399.x.11.i;
A.400.x.11.i; A.401.x.11.i; A.402.x.11.i; A.403.x.11.i; A.404.x.11.i; A.405.x.11.i;
A.406.x.11.i; A.407.x.11.i; A.408.x.11.i; A.409.x.11.i; A.410.x.11.i; A.411.x.11.i;
A.412.x.11.i; A.413.x.11.i; A.414.x.11.i; A.415.x.11.i; A.416.x.11.i; A.417.x.11.i;
A.418.x.11.i; A.419.x.11.i; A.420.x.11.i; A.421.x.11.i; A.422.x.11.i; A.423.x.11.i;
A.424.x.11.i; A.425.x.11.i; A.426.x.11.i; A.427.x.11.i; A.428.x.11.i; A.429.x.11.i;
A.430.x.11.i; A.431.x.11.i; A.432.x.11.i; A.433.x.11.i; A.434.x.11.i; A.435.x.11.i;
A.436.x.11.i; A.437.x.11.i; A.438.x.11.i; A.439.x.11.i; A.440.x.11.i; A.441.x.11.i;
A.442.x.11.i; A.443.x.11.i; A.444.x.11.i; A.445.x.11.i; A.446.x.11.i; A.447.x.11.i;
A.448.x.11.i; A.449.x.11.i; A.450.x.11.i; A.451.x.11.i; A.452.x.11.i; A.453.x.11.i;
A.454.x.11.i; A.455.x.11.i; A.456.x.11.i; A.457.x.11.i; A.458.x.11.i; A.459.x.11.i;
A.460.x.11.i; A.461.x.11.i; A.462.x.11.i; A.463.x.11.i; A.464.x.11.i; A.465.x.11.i;
A.466.x.11.i; A.467.x.11.i; A.468.x.11.i; A.469.x.11.i; A.470.x.11.i; A.471.x.11.i;
A.472.x.11.i; A.473.x.11.i; A.474.x.11.i; A.475.x.11.i; A.476.x.11.i; A.477.x.11.i;
A.478.x.11.i; A.479.x.11.i; A.480.x.11.i; A.481.x.11.i; A.482.x.11.i; A.483.x.11.i;
A.484.x.11.i; A.485.x.11.i; A.486.x.11.i; A.487.x.11.i; A.488.x.11.i; A.489.x.11.i;
A.490.x.11.i; A.491.x.11.i; A.492.x.11.i; A.493.x.11.i; A.494.x.11.i; A.495.x.11.i;
A.496.x.11.i; A.497.x.11.i; A.498.x.11.i; A.499.x.11.i; A.500.x.11.i; A.501.x.11.i;
A.502.x.11.i; A.503.x.11.i; A.504.x.11.i; A.505.x.11.i; A.506.x.11.i; A.507.x.11.i;
A.508.x.11.i; A.509.x.11.i; A.510.x.11.i; A.511.x.11.i; A.512.x.11.i; A.512.x.11.i;
A.513.x.11.i; A.514.x.11.i; A.515.x.11.i; A.516.x.11.i; A.517.x.11.i; A.518.x.11.i;
A.519.x.11.i; A.520.x.11.i; A.521.x.11.i; A.522.x.11.i; A.523.x.11.i; A.524.x.11.i;
A.525.x.11.i; A.526.x.11.i; A.527.x.11.i; A.528.x.11.i; A.529.x.11.i; A.530.x.11.i;
A.531.x.11.i; A.532.x.11.i; A.533.x.11.i; A.534.x.11.i; A.535.x.11.i; A.536.x.11.i;
A.537.x.11.i; A.538.x.11.i; A.539.x.11.i; A.540.x.11.i; A.541.x.11.i; A.542.x.11.i;
A.543.x.11.i; A.544.x.11.i; A.545.x.11.i; A.546.x.11.i; A.547.x.11.i; A.548.x.11.i;
A.549.x.11.i; A.550.x.11.i; A.551.x.11.i; A.552.x.11.i; A.553.x.11.i; A.554.x.11.i;
A.555.x.11.i; A.556.x.11.i; A.557.x.11.i; A.558.x.11.i; A.559.x.11.i; A.560.x.11.i;
A.561.x.11.i; A.562.x.11.i; A.563.x.11.i; A.564.x.11.i; A.565.x.11.i; A.566.x.11.i;
A.567.x.11.i; A.568.x.11.i; A.569.x.11.i; A.570.x.11.i; A.571.x.11.i; A.572.x.11.i;
A.573.x.11.i; A.574.x.11.i; A.575.x.11.i; A.576.x.11.i; A.577.x.11.i; A.578.x.11.i;
A.579.x.11.i; A.580.x.11.i; A.581.x.11.i; A.582.x.11.i; A.583.x.11.i; A.584.x.11.i;
A.585.x.11.i; A.586.x.11.i; A.587.x.11.i; A.588.x.11.i; A.589.x.11.i; A.590.x.11.i;
A.591.x.11.i; A.592.x.11.i; A.593.x.11.i; A.594.x.11.i; A.595.x.11.i; A.596.x.11.i;
A.597.x.11.i; A.598.x.11.i; A.599.x.11.i; A.600.x.11.i; A.601.x.11.i; A.602.x.11.i;
A.603.x.11.i; A.604.x.11.i; A.605.x.11.i; A.606.x.11.i; A.607.x.11.i; A.608.x.11.i;
A.609.x.11.i; A.610.x.11.i; A.611.x.11.i; A.612.x.11.i; A.613.x.11.i; A.614.x.11.i;
A.615.x.11.i; A.616.x.11.i; A.617.x.11.i; A.618.x.11.i; A.619.x.11.i; A.620.x.11.i;
A.621.x.11.i; A.622.x.11.i; A.623.x.11.i; A.624.x.11.i; A.625.x.11.i; A.626.x.11.i;
A.627.x.11.i; A.628.x.11.i; A.629.x.11.i; A.630.x.11.i; A.631.x.11.i; A.632.x.11.i;
A.633.x.11.i; A.634.x.11.i; A.635.x.11.i; A.636.x.11.i; A.637.x.11.i; A.638.x.11.i;
A.639.x.11.i; A.640.x.11.i; A.641.x.11.i; A.642.x.11.i; A.643.x.11.i; A.644.x.11.i;
A.645.x.11.i; A.646.x.11.i; A.647.x.11.i; A.648.x.11.i; A.649.x.11.i; A.650.x.11.i;
A.651.x.11.i; A.652.x.11.i; A.653.x.11.i; A.654.x.11.i; A.655.x.11.i; A.656.x.11.i;
A.657.x.11.i; A.658.x.11.i; A.659.x.11.i; A.660.x.11.i; A.2.y.4.i; A.3.y.4.i; A.4.y.4.i;
A.5.y.4.i; A.6.y.4.i; A.7.y.4.i; A.9.y.4.i; A.10.y.4.i; A.15.y.4.i; A.100.y.4.i;
A.101.y.4.i; A.102.y.4.i; A.103.y.4.i; A.104.y.4.i; A.105.y.4.i; A.106.y.4.i;
A.107.y.4.i; A.108.y.4.i; A.109.y.4.i; A.110.y.4.i; A.111.y.4.i; A.112.y.4.i;
A.113.y.4.i; A.114.y.4.i; A.115.y.4.i; A.116.y.4.i; A.117.y.4.i; A.118.y.4.i;
A.119.y.4.i; A.120.y.4.i; A.121.y.4.i; A.122.y.4.i; A.123.y.4.i; A.124.y.4.i;
A.125.y.4.i; A.126.y.4.i; A.127.y.4.i; A.128.y.4.i; A.129.y.4.i; A.130.y.4.i;
A.131.y.4.i; A.132.y.4.i; A.133.y.4.i; A.134.y.4.i; A.135.y.4.i; A.136.y.4.i;
A.137.y.4.i; A.138.y.4.i; A.139.y.4.i; A.140.y.4.i; A.141.y.4.i; A.142.y.4.i;
A.143.y.4.i; A.144.y.4.i; A.145.y.4.i; A.146.y.4.i; A.147.y.4.i; A.148.y.4.i;
A.149.y.4.i; A.150.y.4.i; A.151.y.4.i; A.152.y.4.i; A.153.y.4.i; A.154.y.4.i;
A.155.y.4.i; A.156.y.4.i; A.157.y.4.i; A.158.y.4.i; A.159.y.4.i; A.160.y.4.i;
A.161.y.4.i; A.162.y.4.i; A.163.y.4.i; A.164.y.4.i; A.165.y.4.i; A.166.y.4.i;
A.167.y.4.i; A.168.y.4.i; A.169.y.4.i; A.170.y.4.i; A.171.y.4.i; A.172.y.4.i;
A.173.y.4.i; A.174.y.4.i; A.175.y.4.i; A.176.y.4.i; A.177.y.4.i; A.178.y.4.i;
A.179.y.4.i; A.180.y.4.i; A.181.y.4.i; A.182.y.4.i; A.183.y.4.i; A.184.y.4.i;
A.185.y.4.i; A.186.y.4.i; A.187.y.4.i; A.188.y.4.i; A.189.y.4.i; A.190.y.4.i;
A.191.y.4.i; A.192.y.4.i; A.193.y.4.i; A.194.y.4.i; A.195.y.4.i; A.196.y.4.i;
A.197.y.4.i; A.198.y.4.i; A.199.y.4.i; A.200.y.4.i; A.201.y.4.i; A.202.y.4.i;
A.203.y.4.i; A.204.y.4.i; A.205.y.4.i; A.206.y.4.i; A.207.y.4.i; A.208.y.4.i;
A.209.y.4.i; A.210.y.4.i; A.211.y.4.i; A.212.y.4.i; A.213.y.4.i; A.214.y.4.i;
A.215.y.4.i; A.216.y.4.i; A.217.y.4.i; A.218.y.4.i; A.219.y.4.i; A.220.y.4.i;
A.221.y.4.i; A.222.y.4.i; A.223.y.4.i; A.224.y.4.i; A.225.y.4.i; A.226.y.4.i;
A.227.y.4.i; A.228.y.4.i; A.229.y.4.i; A.230.y.4.i; A.231.y.4.i; A.232.y.4.i;
A.233.y.4.i; A.234.y.4.i; A.235.y.4.i; A.236.y.4.i; A.237.y.4.i; A.238.y.4.i;
A.239.y.4.i; A.240.y.4.i; A.241.y.4.i; A.242.y.4.i; A.243.y.4.i; A.244.y.4.i;
A.245.y.4.i; A.246.y.4.i; A.247.y.4.i; A.248.y.4.i; A.249.y.4.i; A.250.y.4.i;
A.251.y.4.i; A.252.y.4.i; A.253.y.4.i; A.254.y.4.i; A.255.y.4.i; A.256.y.4.i;
A.257.y.4.i; A.258.y.4.i; A.259.y.4.i; A.260.y.4.i; A.261.y.4.i; A.262.y.4.i;
A.263.y.4.i; A.264.y.4.i; A.265.y.4.i; A.266.y.4.i; A.267.y.4.i; A.268.y.4.i;
A.269.y.4.i; A.270.y.4.i; A.271.y.4.i; A.272.y.4.i; A.273.y.4.i; A.274.y.4.i;
A.275.y.4.i; A.276.y.4.i; A.277.y.4.i; A.278.y.4.i; A.279.y.4.i; A.280.y.4.i;
A.281.y.4.i; A.282.y.4.i; A.283.y.4.i; A.284.y.4.i; A.285.y.4.i; A.286.y.4.i;
A.287.y.4.i; A.288.y.4.i; A.289.y.4.i; A.290.y.4.i; A.291.y.4.i; A.292.y.4.i;
A.293.y.4.i; A.294.y.4.i; A.295.y.4.i; A.296.y.4.i; A.297.y.4.i; A.298.y.4.i;
A.299.y.4.i; A.300.y.4.i; A.301.y.4.i; A.302.y.4.i; A.303.y.4.i; A.304.y.4.i;
A.305.y.4.i; A.306.y.4.i; A.307.y.4.i; A.308.y.4.i; A.309.y.4.i; A.310.y.4.i;
A.311.y.4.i; A.312.y.4.i; A.313.y.4.i; A.314.y.4.i; A.315.y.4.i; A.316.y.4.i;
A.317.y.4.i; A.318.y.4.i; A.319.y.4.i; A.320.y.4.i; A.321.y.4.i; A.323.y.4.i;
A.324.y.4.i; A.325.y.4.i; A.326.y.4.i; A.327.y.4.i; A.328.y.4.i; A.329.y.4.i;
A.330.y.4.i; A.331.y.4.i; A.332.y.4.i; A.333.y.4.i; A.334.y.4.i; A.335.y.4.i;
A.336.y.4.i; A.337.y.4.i; A.338.y.4.i; A.339.y.4.i; A.340.y.4.i; A.341.y.4.i;
A.342.y.4.i; A.343.y.4.i; A.344.y.4.i; A.345.y.4.i; A.346.y.4.i; A.347.y.4.i;
A.348.y.4.i; A.349.y.4.i; A.350.y.4.i; A.351.y.4.i; A.352.y.4.i; A.353.y.4.i;
A.354.y.4.i; A.355.y.4.i; A.356.y.4.i; A.357.y.4.i; A.358.y.4.i; A.359.y.4.i;
A.360.y.4.i; A.361.y.4.i; A.362.y.4.i; A.363.y.4.i; A.364.y.4.i; A.365.y.4.i;
A.366.y.4.i; A.367.y.4.i; A.368.y.4.i; A.369.y.4.i; A.370.y.4.i; A.371.y.4.i;
A.372.y.4.i; A.373.y.4.i; A.374.y.4.i; A.375.y.4.i; A.376.y.4.i; A.377.y.4.i;
A.378.y.4.i; A.379.y.4.i; A.380.y.4.i; A.381.y.4.i; A.382.y.4.i; A.383.y.4.i;
A.384.y.4.i; A.385.y.4.i; A.386.y.4.i; A.387.y.4.i; A.388.y.4.i; A.389.y.4.i;
A.390.y.4.i; A.391.y.4.i; A.392.y.4.i; A.393.y.4.i; A.394.y.4.i; A.395.y.4.i;
A.396.y.4.i; A.397.y.4.i; A.398.y.4.i; A.399.y.4.i; A.400.y.4.i; A.401.y.4.i;
A.402.y.4.i; A.403.y.4.i; A.404.y.4.i; A.405.y.4.i; A.406.y.4.i; A.407.y.4.i;
A.408.y.4.i; A.409.y.4.i; A.410.y.4.i; A.411.y.4.i; A.412.y.4.i; A.413.y.4.i;
A.414.y.4.i; A.415.y.4.i; A.416.y.4.i; A.417.y.4.i; A.418.y.4.i; A.419.y.4.i;
A.420.y.4.i; A.421.y.4.i; A.422.y.4.i; A.423.y.4.i; A.424.y.4.i; A.425.y.4.i;
A.426.y.4.i; A.427.y.4.i; A.428.y.4.i; A.429.y.4.i; A.430.y.4.i; A.431.y.4.i;
A.432.y.4.i; A.433.y.4.i; A.434.y.4.i; A.435.y.4.i; A.436.y.4.i; A.437.y.4.i;
A.438.y.4.i; A.439.y.4.i; A.440.y.4.i; A.441.y.4.i; A.442.y.4.i; A.443.y.4.i;
A.444.y.4.i; A.445.y.4.i; A.446.y.4.i; A.447.y.4.i; A.448.y.4.i; A.449.y.4.i;
A.450.y.4.i; A.451.y.4.i; A.452.y.4.i; A.453.y.4.i; A.454.y.4.i; A.455.y.4.i;
A.456.y.4.i; A.457.y.4.i; A.458.y.4.i; A.459.y.4.i; A.460.y.4.i; A.461.y.4.i;
A.462.y.4.i; A.463.y.4.i; A.464.y.4.i; A.465.y.4.i; A.466.y.4.i; A.467.y.4.i;
A.468.y.4.i; A.469.y.4.i; A.470.y.4.i; A.471.y.4.i; A.472.y.4.i; A.473.y.4.i;
A.474.y.4.i; A.475.y.4.i; A.476.y.4.i; A.477.y.4.i; A.478.y.4.i; A.479.y.4.i;
A.480.y.4.i; A.481.y.4.i; A.482.y.4.i; A.483.y.4.i; A.484.y.4.i; A.485.y.4.i;
A.486.y.4.i; A.487.y.4.i; A.488.y.4.i; A.489.y.4.i; A.490.y.4.i; A.491.y.4.i;
A.492.y.4.i; A.493.y.4.i; A.494.y.4.i; A.495.y.4.i; A.496.y.4.i; A.497.y.4.i;
A.498.y.4.i; A.499.y.4.i; A.500.y.4.i; A.501.y.4.i; A.502.y.4.i; A.503.y.4.i;
A.504.y.4.i; A.505.y.4.i; A.506.y.4.i; A.507.y.4.i; A.508.y.4.i; A.509.y.4.i;
A.510.y.4.i; A.511.y.4.i; A.512.y.4.i; A.512.y.4.i; A.513.y.4.i; A.514.y.4.i;
A.515.y.4.i; A.516.y.4.i; A.517.y.4.i; A.518.y.4.i; A.519.y.4.i; A.520.y.4.i;
A.521.y.4.i; A.522.y.4.i; A.523.y.4.i; A.524.y.4.i; A.525.y.4.i; A.526.y.4.i;
A.527.y.4.i; A.528.y.4.i; A.529.y.4.i; A.530.y.4.i; A.531.y.4.i; A.532.y.4.i;
A.533.y.4.i; A.534.y.4.i; A.535.y.4.i; A.536.y.4.i; A.537.y.4.i; A.538.y.4.i;
A.539.y.4.i; A.540.y.4.i; A.541.y.4.i; A.542.y.4.i; A.543.y.4.i; A.544.y.4.i;
A.545.y.4.i; A.546.y.4.i; A.547.y.4.i; A.548.y.4.i; A.549.y.4.i; A.550.y.4.i;
A.551.y.4.i; A.552.y.4.i; A.553.y.4.i; A.554.y.4.i; A.555.y.4.i; A.556.y.4.i;
A.557.y.4.i; A.558.y.4.i; A.559.y.4.i; A.560.y.4.i; A.561.y.4.i; A.562.y.4.i;
A.563.y.4.i; A.564.y.4.i; A.565.y.4.i; A.566.y.4.i; A.567.y.4.i; A.568.y.4.i;
A.569.y.4.i; A.570.y.4.i; A.571.y.4.i; A.572.y.4.i; A.573.y.4.i; A.574.y.4.i;
A.575.y.4.i; A.576.y.4.i; A.577.y.4.i; A.578.y.4.i; A.579.y.4.i; A.580.y.4.i;
A.581.y.4.i; A.582.y.4.i; A.583.y.4.i; A.584.y.4.i; A.585.y.4.i; A.586.y.4.i;
A.587.y.4.i; A.588.y.4.i; A.589.y.4.i; A.590.y.4.i; A.591.y.4.i; A.592.y.4.i;
A.593.y.4.i; A.594.y.4.i; A.595.y.4.i; A.596.y.4.i; A.597.y.4.i; A.598.y.4.i;
A.599.y.4.i; A.600.y.4.i; A.601.y.4.i; A.602.y.4.i; A.603.y.4.i; A.604.y.4.i;
A.605.y.4.i; A.606.y.4.i; A.607.y.4.i; A.608.y.4.i; A.609.y.4.i; A.610.y.4.i;
A.611.y.4.i; A.612.y.4.i; A.613.y.4.i; A.614.y.4.i; A.615.y.4.i; A.616.y.4.i;
A.617.y.4.i; A.618.y.4.i; A.619.y.4.i; A.620.y.4.i; A.621.y.4.i; A.622.y.4.i;
A.623.y.4.i; A.624.y.4.i; A.625.y.4.i; A.626.y.4.i; A.627.y.4.i; A.628.y.4.i;
A.629.y.4.i; A.630.y.4.i; A.631.y.4.i; A.632.y.4.i; A.633.y.4.i; A.634.y.4.i;
A.635.y.4.i; A.636.y.4.i; A.637.y.4.i; A.638.y.4.i; A.639.y.4.i; A.640.y.4.i;
A.641.y.4.i; A.642.y.4.i; A.643.y.4.i; A.644.y.4.i; A.645.y.4.i; A.646.y.4.i;
A.647.y.4.i; A.648.y.4.i; A.649.y.4.i; A.650.y.4.i; A.651.y.4.i; A.652.y.4.i;
A.653.y.4.i; A.654.y.4.i; A.655.y.4.i; A.656.y.4.i; A.657.y.4.i; A.658.y.4.i;
A.659.y.4.i; A.660.y.4.i; A.2.y.11.i; A.3.y.11.i; A.4.y.11.i; A.5.y.11.i; A.6.y.11.i;
A.7.y.11.i; A.9.y.11.i; A.10.y.11.i; A.15.y.11.i; A.100.y.11.i; A.101.y.11.i;
A.102.y.11.i; A.103.y.11.i; A.104.y.11.i; A.105.y.11.i; A.106.y.11.i; A.107.y.11.i;
A.108.y.11.i; A.109.y.11.i; A.110.y.11.i; A.111.y.11.i; A.112.y.11.i; A.113.y.11.i;
A.114.y.11.i; A.115.y.11.i; A.116.y.11.i; A.117.y.11.i; A.118.y.11.i; A.119.y.11.i;
A.120.y.11.i; A.121.y.11.i; A.122.y.11.i; A.123.y.11.i; A.124.y.11.i; A.125.y.11.i;
A.126.y.11.i; A.127.y.11.i; A.128.y.11.i; A.129.y.11.i; A.130.y.11.i; A.131.y.11.i;
A.132.y.11.i; A.133.y.11.i; A.134.y.11.i; A.135.y.11.i; A.136.y.11.i; A.137.y.11.i;
A.138.y.11.i; A.139.y.11.i; A.140.y.11.i; A.141.y.11.i; A.142.y.11.i; A.143.y.11.i;
A.144.y.11.i; A.145.y.11.i; A.146.y.11.i; A.147.y.11.i; A.148.y.11.i; A.149.y.11.i;
A.150.y.11.i; A.151.y.11.i; A.152.y.11.i; A.153.y.11.i; A.154.y.11.i; A.155.y.11.i;
A.156.y.11.i; A.157.y.11.i; A.158.y.11.i; A.159.y.11.i; A.160.y.11.i; A.161.y.11.i;
A.162.y.11.i; A.163.y.11.i; A.164.y.11.i; A.165.y.11.i; A.166.y.11.i; A.167.y.11.i;
A.168.y.11.i; A.169.y.11.i; A.170.y.11.i; A.171.y.11.i; A.172.y.11.i; A.173.y.11.i;
A.174.y.11.i; A.175.y.11.i; A.176.y.11.i; A.177.y.11.i; A.178.y.11.i; A.179.y.11.i;
A.180.y.11.i; A.181.y.11.i; A.182.y.11.i; A.183.y.11.i; A.184.y.11.i; A.185.y.11.i;
A.186.y.11.i; A.187.y.11.i; A.188.y.11.i; A.189.y.11.i; A.190.y.11.i; A.191.y.11.i;
A.192.y.11.i; A.193.y.11.i; A.194.y.11.i; A.195.y.11.i; A.196.y.11.i; A.197.y.11.i;
A.198.y.11.i; A.199.y.11.i; A.200.y.11.i; A.201.y.11.i; A.202.y.11.i; A.203.y.11.i;
A.204.y.11.i; A.205.y.11.i; A.206.y.11.i; A.207.y.11.i; A.208.y.11.i; A.209.y.11.i;
A.210.y.11.i; A.211.y.11.i; A.212.y.11.i; A.213.y.11.i; A.214.y.11.i; A.215.y.11.i;
A.216.y.11.i; A.217.y.11.i; A.218.y.11.i; A.219.y.11.i; A.220.y.11.i; A.221.y.11.i;
A.222.y.11.i; A.223.y.11.i; A.224.y.11.i; A.225.y.11.i; A.226.y.11.i; A.227.y.11.i;
A.228.y.11.i; A.229.y.11.i; A.230.y.11.i; A.231.y.11.i; A.232.y.11.i; A.233.y.11.i;
A.234.y.11.i; A.235.y.11.i; A.236.y.11.i; A.237.y.11.i; A.238.y.11.i; A.239.y.11.i;
A.240.y.11.i; A.241.y.11.i; A.242.y.11.i; A.243.y.11.i; A.244.y.11.i; A.245.y.11.i;
A.246.y.11.i; A.247.y.11.i; A.248.y.11.i; A.249.y.11.i; A.250.y.11.i; A.251.y.11.i;
A.252.y.11.i; A.253.y.11.i; A.254.y.11.i; A.255.y.11.i; A.256.y.11.i; A.257.y.11.i;
A.258.y.11.i; A.259.y.11.i; A.260.y.11.i; A.261.y.11.i; A.262.y.11.i; A.263.y.11.i;
A.264.y.11.i; A.265.y.11.i; A.266.y.11.i; A.267.y.11.i; A.268.y.11.i; A.269.y.11.i;
A.270.y.11.i; A.271.y.11.i; A.272.y.11.i; A.273.y.11.i; A.274.y.11.i; A.275.y.11.i;
A.276.y.11.i; A.277.y.11.i; A.278.y.11.i; A.279.y.11.i; A.280.y.11.i; A.281.y.11.i;
A.282.y.11.i; A.283.y.11.i; A.284.y.11.i; A.285.y.11.i; A.286.y.11.i; A.287.y.11.i;
A.288.y.11.i; A.289.y.11.i; A.290.y.11.i; A.291.y.11.i; A.292.y.11.i; A.293.y.11.i;
A.294.y.11.i; A.295.y.11.i; A.296.y.11.i; A.297.y.11.i; A.298.y.11.i; A.299.y.11.i;
A.300.y.11.i; A.301.y.11.i; A.302.y.11.i; A.303.y.11.i; A.304.y.11.i; A.305.y.11.i;
A.306.y.11.i; A.307.y.11.i; A.308.y.11.i; A.309.y.11.i; A.310.y.11.i; A.311.y.11.i;
A.312.y.11.i; A.313.y.11.i; A.314.y.11.i; A.315.y.11.i; A.316.y.11.i; A.317.y.11.i;
A.318.y.11.i; A.319.y.11.i; A.320.y.11.i; A.321.y.11.i; A.323.y.11.i; A.324.y.11.i;
A.325.y.11.i; A.326.y.11.i; A.327.y.11.i; A.328.y.11.i; A.329.y.11.i; A.330.y.11.i;
A.331.y.11.i; A.332.y.11.i; A.333.y.11.i; A.334.y.11.i; A.335.y.11.i; A.336.y.11.i;
A.337.y.11.i; A.338.y.11.i; A.339.y.11.i; A.340.y.11.i; A.341.y.11.i; A.342.y.11.i;
A.343.y.11.i; A.344.y.11.i; A.345.y.11.i; A.346.y.11.i; A.347.y.11.i; A.348.y.11.i;
A.349.y.11.i; A.350.y.11.i; A.351.y.11.i; A.352.y.11.i; A.353.y.11.i; A.354.y.11.i;
A.355.y.11.i; A.356.y.11.i; A.357.y.11.i; A.358.y.11.i; A.359.y.11.i; A.360.y.11.i;
A.361.y.11.i; A.362.y.11.i; A.363.y.11.i; A.364.y.11.i; A.365.y.11.i; A.366.y.11.i;
A.367.y.11.i; A.368.y.11.i; A.369.y.11.i; A.370.y.11.i; A.371.y.11.i; A.372.y.11.i;
A.373.y.11.i; A.374.y.11.i; A.375.y.11.i; A.376.y.11.i; A.377.y.11.i; A.378.y.11.i;
A.379.y.11.i; A.380.y.11.i; A.381.y.11.i; A.382.y.11.i; A.383.y.11.i; A.384.y.11.i;
A.385.y.11.i; A.386.y.11.i; A.387.y.11.i; A.388.y.11.i; A.389.y.11.i; A.390.y.11.i;
A.391.y.11.i; A.392.y.11.i; A.393.y.11.i; A.394.y.11.i; A.395.y.11.i; A.396.y.11.i;
A.397.y.11.i; A.398.y.11.i; A.399.y.11.i; A.400.y.11.i; A.401.y.11.i; A.402.y.11.i;
A.403.y.11.i; A.404.y.11.i; A.405.y.11.i; A.406.y.11.i; A.407.y.11.i; A.408.y.11.i;
A.409.y.11.i; A.410.y.11.i; A.411.y.11.i; A.412.y.11.i; A.413.y.11.i; A.414.y.11.i;
A.415.y.11.i; A.416.y.11.i; A.417.y.11.i; A.418.y.11.i; A.419.y.11.i; A.420.y.11.i;
A.421.y.11.i; A.422.y.11.i; A.423.y.11.i; A.424.y.11.i; A.425.y.11.i; A.426.y.11.i;
A.427.y.11.i; A.428.y.11.i; A.429.y.11.i; A.430.y.11.i; A.431.y.11.i; A.432.y.11.i;
A.433.y.11.i; A.434.y.11.i; A.435.y.11.i; A.436.y.11.i; A.437.y.11.i; A.438.y.11.i;
A.439.y.11.i; A.440.y.11.i; A.441.y.11.i; A.442.y.11.i; A.443.y.11.i; A.444.y.11.i;
A.445.y.11.i; A.446.y.11.i; A.447.y.11.i; A.448.y.11.i; A.449.y.11.i; A.450.y.11.i;
A.451.y.11.i; A.452.y.11.i; A.453.y.11.i; A.454.y.11.i; A.455.y.11.i; A.456.y.11.i;
A.457.y.11.i; A.458.y.11.i; A.459.y.11.i; A.460.y.11.i; A.461.y.11.i; A.462.y.11.i;
A.463.y.11.i; A.464.y.11.i; A.465.y.11.i; A.466.y.11.i; A.467.y.11.i; A.468.y.11.i;
A.469.y.11.i; A.470.y.11.i; A.471.y.11.i; A.472.y.11.i; A.473.y.11.i; A.474.y.11.i;
A.475.y.11.i; A.476.y.11.i; A.477.y.11.i; A.478.y.11.i; A.479.y.11.i; A.480.y.11.i;
A.481.y.11.i; A.482.y.11.i; A.483.y.11.i; A.484.y.11.i; A.485.y.11.i; A.486.y.11.i;
A.487.y.11.i; A.488.y.11.i; A.489.y.11.i; A.490.y.11.i; A.491.y.11.i; A.492.y.11.i;
A.493.y.11.i; A.494.y.11.i; A.495.y.11.i; A.496.y.11.i; A.497.y.11.i; A.498.y.11.i;
A.499.y.11.i; A.500.y.11.i; A.501.y.11.i; A.502.y.11.i; A.503.y.11.i; A.504.y.11.i;
A.505.y.11.i; A.506.y.11.i; A.507.y.11.i; A.508.y.11.i; A.509.y.11.i; A.510.y.11.i;
A.511.y.11.i; A.512.y.11.i; A.512.y.11.i; A.513.y.11.i; A.514.y.11.i; A.515.y.11.i;
A.516.y.11.i; A.517.y.11.i; A.518.y.11.i; A.519.y.11.i; A.520.y.11.i; A.521.y.11.i;
A.522.y.11.i; A.523.y.11.i; A.524.y.11.i; A.525.y.11.i; A.526.y.11.i; A.527.y.11.i;
A.528.y.11.i; A.529.y.11.i; A.530.y.11.i; A.531.y.11.i; A.532.y.11.i; A.533.y.11.i;
A.534.y.11.i; A.535.y.11.i; A.536.y.11.i; A.537.y.11.i; A.538.y.11.i; A.539.y.11.i;
A.540.y.11.i; A.541.y.11.i; A.542.y.11.i; A.543.y.11.i; A.544.y.11.i; A.545.y.11.i;
A.546.y.11.i; A.547.y.11.i; A.548.y.11.i; A.549.y.11.i; A.550.y.11.i; A.551.y.11.i;
A.552.y.11.i; A.553.y.11.i; A.554.y.11.i; A.555.y.11.i; A.556.y.11.i; A.557.y.11.i;
A.558.y.11.i; A.559.y.11.i; A.560.y.11.i; A.561.y.11.i; A.562.y.11.i; A.563.y.11.i;
A.564.y.11.i; A.565.y.11.i; A.566.y.11.i; A.567.y.11.i; A.568.y.11.i; A.569.y.11.i;
A.570.y.11.i; A.571.y.11.i; A.572.y.11.i; A.573.y.11.i; A.574.y.11.i; A.575.y.11.i;
A.576.y.11.i; A.577.y.11.i; A.578.y.11.i; A.579.y.11.i; A.580.y.11.i; A.581.y.11.i;
A.582.y.11.i; A.583.y.11.i; A.584.y.11.i; A.585.y.11.i; A.586.y.11.i; A.587.y.11.i;
A.588.y.11.i; A.589.y.11.i; A.590.y.11.i; A.591.y.11.i; A.592.y.11.i; A.593.y.11.i;
A.594.y.11.i; A.595.y.11.i; A.596.y.11.i; A.597.y.11.i; A.598.y.11.i; A.599.y.11.i;
A.600.y.11.i; A.601.y.11.i; A.602.y.11.i; A.603.y.11.i; A.604.y.11.i; A.605.y.11.i;
A.606.y.11.i; A.607.y.11.i; A.608.y.11.i; A.609.y.11.i; A.610.y.11.i; A.611.y.11.i;
A.612.y.11.i; A.613.y.11.i; A.614.y.11.i; A.615.y.11.i; A.616.y.11.i; A.617.y.11.i;
A.618.y.11.i; A.619.y.11.i; A.620.y.11.i; A.621.y.11.i; A.622.y.11.i; A.623.y.11.i;
A.624.y.11.i; A.625.y.11.i; A.626.y.11.i; A.627.y.11.i; A.628.y.11.i; A.629.y.11.i;
A.630.y.11.i; A.631.y.11.i; A.632.y.11.i; A.633.y.11.i; A.634.y.11.i; A.635.y.11.i;
A.636.y.11.i; A.637.y.11.i; A.638.y.11.i; A.639.y.11.i; A.640.y.11.i; A.641.y.11.i;
A.642.y.11.i; A.643.y.11.i; A.644.y.11.i; A.645.y.11.i; A.646.y.11.i; A.647.y.11.i;
A.648.y.11.i; A.649.y.11.i; A.650.y.11.i; A.651.y.11.i; A.652.y.11.i; A.653.y.11.i;
A.654.y.11.i; A.655.y.11.i; A.656.y.11.i; A.657.y.11.i; A.658.y.11.i; A.659.y.11.i;
A.660.y.11.i; A.2.z.4.i; A.3.z.4.i; A.4.z.4.i; A.5.z.4.i; A.6.z.4.i; A.7.z.4.i; A.9.z.4.i;
A.10.z.4.i; A.15.z.4.i; A.100.z.4.i; A.101.z.4.i; A.102.z.4.i; A.103.z.4.i; A.104.z.4.i;
A.105.z.4.i; A.106.z.4.i; A.107.z.4.i; A.108.z.4.i; A.109.z.4.i; A.110.z.4.i;
A.111.z.4.i; A.112.z.4.i; A.113.z.4.i; A.114.z.4.i; A.115.z.4.i; A.116.z.4.i;
A.117.z.4.i; A.118.z.4.i; A.119.z.4.i; A.120.z.4.i; A.121.z.4.i; A.122.z.4.i;
A.123.z.4.i; A.124.z.4.i; A.125.z.4.i; A.126.z.4.i; A.127.z.4.i; A.128.z.4.i;
A.129.z.4.i; A.130.z.4.i; A.131.z.4.i; A.132.z.4.i; A.133.z.4.i; A.134.z.4.i;
A.135.z.4.i; A.136.z.4.i; A.137.z.4.i; A.138.z.4.i; A.139.z.4.i; A.140.z.4.i;
A.141.z.4.i; A.142.z.4.i; A.143.z.4.i; A.144.z.4.i; A.145.z.4.i; A.146.z.4.i;
A.147.z.4.i; A.148.z.4.i; A.149.z.4.i; A.150.z.4.i; A.151.z.4.i; A.152.z.4.i;
A.153.z.4.i; A.154.z.4.i; A.155.z.4.i; A.156.z.4.i; A.157.z.4.i; A.158.z.4.i;
A.159.z.4.i; A.160.z.4.i; A.161.z.4.i; A.162.z.4.i; A.163.z.4.i; A.164.z.4.i;
A.165.z.4.i; A.166.z.4.i; A.167.z.4.i; A.168.z.4.i; A.169.z.4.i; A.170.z.4.i;
A.171.z.4.i; A.172.z.4.i; A.173.z.4.i; A.174.z.4.i; A.175.z.4.i; A.176.z.4.i;
A.177.z.4.i; A.178.z.4.i; A.179.z.4.i; A.180.z.4.i; A.181.z.4.i; A.182.z.4.i;
A.183.z.4.i; A.184.z.4.i; A.185.z.4.i; A.186.z.4.i; A.187.z.4.i; A.188.z.4.i;
A.189.z.4.i; A.190.z.4.i; A.191.z.4.i; A.192.z.4.i; A.193.z.4.i; A.194.z.4.i;
A.195.z.4.i; A.196.z.4.i; A.197.z.4.i; A.198.z.4.i; A.199.z.4.i; A.200.z.4.i;
A.201.z.4.i; A.202.z.4.i; A.203.z.4.i; A.204.z.4.i; A.205.z.4.i; A.206.z.4.i;
A.207.z.4.i; A.208.z.4.i; A.209.z.4.i; A.210.z.4.i; A.211.z.4.i; A.212.z.4.i;
A.213.z.4.i; A.214.z.4.i; A.215.z.4.i; A.216.z.4.i; A.217.z.4.i; A.218.z.4.i;
A.219.z.4.i; A.220.z.4.i; A.221.z.4.i; A.222.z.4.i; A.223.z.4.i; A.224.z.4.i;
A.225.z.4.i; A.226.z.4.i; A.227.z.4.i; A.228.z.4.i; A.229.z.4.i; A.230.z.4.i;
A.231.z.4.i; A.232.z.4.i; A.233.z.4.i; A.234.z.4.i; A.235.z.4.i; A.236.z.4.i;
A.237.z.4.i; A.238.z.4.i; A.239.z.4.i; A.240.z.4.i; A.241.z.4.i; A.242.z.4.i;
A.243.z.4.i; A.244.z.4.i; A.245.z.4.i; A.246.z.4.i; A.247.z.4.i; A.248.z.4.i;
A.249.z.4.i; A.250.z.4.i; A.251.z.4.i; A.252.z.4.i; A.253.z.4.i; A.254.z.4.i;
A.255.z.4.i; A.256.z.4.i; A.257.z.4.i; A.258.z.4.i; A.259.z.4.i; A.260.z.4.i;
A.261.z.4.i; A.262.z.4.i; A.263.z.4.i; A.264.z.4.i; A.265.z.4.i; A.266.z.4.i;
A.267.z.4.i; A.268.z.4.i; A.269.z.4.i; A.270.z.4.i; A.271.z.4.i; A.272.z.4.i;
A.273.z.4.i; A.274.z.4.i; A.275.z.4.i; A.276.z.4.i; A.277.z.4.i; A.278.z.4.i;
A.279.z.4.i; A.280.z.4.i; A.281.z.4.i; A.282.z.4.i; A.283.z.4.i; A.284.z.4.i;
A.285.z.4.i; A.286.z.4.i; A.287.z.4.i; A.288.z.4.i; A.289.z.4.i; A.290.z.4.i;
A.291.z.4.i; A.292.z.4.i; A.293.z.4.i; A.294.z.4.i; A.295.z.4.i; A.296.z.4.i;
A.297.z.4.i; A.298.z.4.i; A.299.z.4.i; A.300.z.4.i; A.301.z.4.i; A.302.z.4.i;
A.303.z.4.i; A.304.z.4.i; A.305.z.4.i; A.306.z.4.i; A.307.z.4.i; A.308.z.4.i;
A.309.z.4.i; A.310.z.4.i; A.311.z.4.i; A.312.z.4.i; A.313.z.4.i; A.314.z.4.i;
A.315.z.4.i; A.316.z.4.i; A.317.z.4.i; A.318.z.4.i; A.319.z.4.i; A.320.z.4.i;
A.321.z.4.i; A.323.z.4.i; A.324.z.4.i; A.325.z.4.i; A.326.z.4.i; A.327.z.4.i;
A.328.z.4.i; A.329.z.4.i; A.330.z.4.i; A.331.z.4.i; A.332.z.4.i; A.333.z.4.i;
A.334.z.4.i; A.335.z.4.i; A.336.z.4.i; A.337.z.4.i; A.338.z.4.i; A.339.z.4.i;
A.340.z.4.i; A.341.z.4.i; A.342.z.4.i; A.343.z.4.i; A.344.z.4.i; A.345.z.4.i;
A.346.z.4.i; A.347.z.4.i; A.348.z.4.i; A.349.z.4.i; A.350.z.4.i; A.351.z.4.i;
A.352.z.4.i; A.353.z.4.i; A.354.z.4.i; A.355.z.4.i; A.356.z.4.i; A.357.z.4.i;
A.358.z.4.i; A.359.z.4.i; A.360.z.4.i; A.361.z.4.i; A.362.z.4.i; A.363.z.4.i;
A.364.z.4.i; A.365.z.4.i; A.366.z.4.i; A.367.z.4.i; A.368.z.4.i; A.369.z.4.i;
A.370.z.4.i; A.371.z.4.i; A.372.z.4.i; A.373.z.4.i; A.374.z.4.i; A.375.z.4.i;
A.376.z.4.i; A.377.z.4.i; A.378.z.4.i; A.379.z.4.i; A.380.z.4.i; A.381.z.4.i;
A.382.z.4.i; A.383.z.4.i; A.384.z.4.i; A.385.z.4.i; A.386.z.4.i; A.387.z.4.i;
A.388.z.4.i; A.389.z.4.i; A.390.z.4.i; A.391.z.4.i; A.392.z.4.i; A.393.z.4.i;
A.394.z.4.i; A.395.z.4.i; A.396.z.4.i; A.397.z.4.i; A.398.z.4.i; A.399.z.4.i;
A.400.z.4.i; A.401.z.4.i; A.402.z.4.i; A.403.z.4.i; A.404.z.4.i; A.405.z.4.i;
A.406.z.4.i; A.407.z.4.i; A.408.z.4.i; A.409.z.4.i; A.410.z.4.i; A.411.z.4.i;
A.412.z.4.i; A.413.z.4.i; A.414.z.4.i; A.415.z.4.i; A.416.z.4.i; A.417.z.4.i;
A.418.z.4.i; A.419.z.4.i; A.420.z.4.i; A.421.z.4.i; A.422.z.4.i; A.423.z.4.i;
A.424.z.4.i; A.425.z.4.i; A.426.z.4.i; A.427.z.4.i; A.428.z.4.i; A.429.z.4.i;
A.430.z.4.i; A.431.z.4.i; A.432.z.4.i; A.433.z.4.i; A.434.z.4.i; A.435.z.4.i;
A.436.z.4.i; A.437.z.4.i; A.438.z.4.i; A.439.z.4.i; A.440.z.4.i; A.441.z.4.i;
A.442.z.4.i; A.443.z.4.i; A.444.z.4.i; A.445.z.4.i; A.446.z.4.i; A.447.z.4.i;
A.448.z.4.i; A.449.z.4.i; A.450.z.4.i; A.451.z.4.i; A.452.z.4.i; A.453.z.4.i;
A.454.z.4.i; A.455.z.4.i; A.456.z.4.i; A.457.z.4.i; A.458.z.4.i; A.459.z.4.i;
A.460.z.4.i; A.461.z.4.i; A.462.z.4.i; A.463.z.4.i; A.464.z.4.i; A.465.z.4.i;
A.466.z.4.i; A.467.z.4.i; A.468.z.4.i; A.469.z.4.i; A.470.z.4.i; A.471.z.4.i;
A.472.z.4.i; A.473.z.4.i; A.474.z.4.i; A.475.z.4.i; A.476.z.4.i; A.477.z.4.i;
A.478.z.4.i; A.479.z.4.i; A.480.z.4.i; A.481.z.4.i; A.482.z.4.i; A.483.z.4.i;
A.484.z.4.i; A.485.z.4.i; A.486.z.4.i; A.487.z.4.i; A.488.z.4.i; A.489.z.4.i;
A.490.z.4.i; A.491.z.4.i; A.492.z.4.i; A.493.z.4.i; A.494.z.4.i; A.495.z.4.i;
A.496.z.4.i; A.497.z.4.i; A.498.z.4.i; A.499.z.4.i; A.500.z.4.i; A.501.z.4.i;
A.502.z.4.i; A.503.z.4.i; A.504.z.4.i; A.505.z.4.i; A.506.z.4.i; A.507.z.4.i;
A.508.z.4.i; A.509.z.4.i; A.510.z.4.i; A.511.z.4.i; A.512.z.4.i; A.512.z.4.i;
A.513.z.4.i; A.514.z.4.i; A.515.z.4.i; A.516.z.4.i; A.517.z.4.i; A.518.z.4.i;
A.519.z.4.i; A.520.z.4.i; A.521.z.4.i; A.522.z.4.i; A.523.z.4.i; A.524.z.4.i;
A.525.z.4.i; A.526.z.4.i; A.527.z.4.i; A.528.z.4.i; A.529.z.4.i; A.530.z.4.i;
A.531.z.4.i; A.532.z.4.i; A.533.z.4.i; A.534.z.4.i; A.535.z.4.i; A.536.z.4.i;
A.537.z.4.i; A.538.z.4.i; A.539.z.4.i; A.540.z.4.i; A.541.z.4.i; A.542.z.4.i;
A.543.z.4.i; A.544.z.4.i; A.545.z.4.i; A.546.z.4.i; A.547.z.4.i; A.548.z.4.i;
A.549.z.4.i; A.550.z.4.i; A.551.z.4.i; A.552.z.4.i; A.553.z.4.i; A.554.z.4.i;
A.555.z.4.i; A.556.z.4.i; A.557.z.4.i; A.558.z.4.i; A.559.z.4.i; A.560.z.4.i;
A.561.z.4.i; A.562.z.4.i; A.563.z.4.i; A.564.z.4.i; A.565.z.4.i; A.566.z.4.i;
A.567.z.4.i; A.568.z.4.i; A.569.z.4.i; A.570.z.4.i; A.571.z.4.i; A.572.z.4.i;
A.573.z.4.i; A.574.z.4.i; A.575.z.4.i; A.576.z.4.i; A.577.z.4.i; A.578.z.4.i;
A.579.z.4.i; A.580.z.4.i; A.581.z.4.i; A.582.z.4.i; A.583.z.4.i; A.584.z.4.i;
A.585.z.4.i; A.586.z.4.i; A.587.z.4.i; A.588.z.4.i; A.589.z.4.i; A.590.z.4.i;
A.591.z.4.i; A.592.z.4.i; A.593.z.4.i; A.594.z.4.i; A.595.z.4.i; A.596.z.4.i;
A.597.z.4.i; A.598.z.4.i; A.599.z.4.i; A.600.z.4.i; A.601.z.4.i; A.602.z.4.i;
A.603.z.4.i; A.604.z.4.i; A.605.z.4.i; A.606.z.4.i; A.607.z.4.i; A.608.z.4.i;
A.609.z.4.i; A.610.z.4.i; A.611.z.4.i; A.612.z.4.i; A.613.z.4.i; A.614.z.4.i;
A.615.z.4.i; A.616.z.4.i; A.617.z.4.i; A.618.z.4.i; A.619.z.4.i; A.620.z.4.i;
A.621.z.4.i; A.622.z.4.i; A.623.z.4.i; A.624.z.4.i; A.625.z.4.i; A.626.z.4.i;
A.627.z.4.i; A.628.z.4.i; A.629.z.4.i; A.630.z.4.i; A.631.z.4.i; A.632.z.4.i;
A.633.z.4.i; A.634.z.4.i; A.635.z.4.i; A.636.z.4.i; A.637.z.4.i; A.638.z.4.i;
A.639.z.4.i; A.640.z.4.i; A.641.z.4.i; A.642.z.4.i; A.643.z.4.i; A.644.z.4.i;
A.645.z.4.i; A.646.z.4.i; A.647.z.4.i; A.648.z.4.i; A.649.z.4.i; A.650.z.4.i;
A.651.z.4.i; A.652.z.4.i; A.653.z.4.i; A.654.z.4.i; A.655.z.4.i; A.656.z.4.i;
A.657.z.4.i; A.658.z.4.i; A.659.z.4.i; A.660.z.4.i; A.2.z.11.i; A.3.z.11.i; A.4.z.11.i;
A.5.z.11.i; A.6.z.11.i; A.7.z.11.i; A.9.z.11.i; A.10.z.11.i; A.15.z.11.i; A.100.z.11.i;
A.101.z.11.i; A.102.z.11.i; A.103.z.11.i; A.104.z.11.i; A.105.z.11.i; A.106.z.11.i;
A.107.z.11.i; A.108.z.11.i; A.109.z.11.i; A.110.z.11.i; A.111.z.11.i; A.112.z.11.i;
A.113.z.11.i; A.114.z.11.i; A.115.z.11.i; A.116.z.11.i; A.117.z.11.i; A.118.z.11.i;
A.119.z.11.i; A.120.z.11.i; A.121.z.11.i; A.122.z.11.i; A.123.z.11.i; A.124.z.11.i;
A.125.z.11.i; A.126.z.11.i; A.127.z.11.i; A.128.z.11.i; A.129.z.11.i; A.130.z.11.i;
A.131.z.11.i; A.132.z.11.i; A.133.z.11.i; A.134.z.11.i; A.135.z.11.i; A.136.z.11.i;
A.137.z.11.i; A.138.z.11.i; A.139.z.11.i; A.140.z.11.i; A.141.z.11.i; A.142.z.11.i;
A.143.z.11.i; A.144.z.11.i; A.145.z.11.i; A.146.z.11.i; A.147.z.11.i; A.148.z.11.i;
A.149.z.11.i; A.150.z.11.i; A.151.z.11.i; A.152.z.11.i; A.153.z.11.i; A.154.z.11.i;
A.155.z.11.i; A.156.z.11.i; A.157.z.11.i; A.158.z.11.i; A.159.z.11.i; A.160.z.11.i;
A.161.z.11.i; A.162.z.11.i; A.163.z.11.i; A.164.z.11.i; A.165.z.11.i; A.166.z.11.i;
A.167.z.11.i; A.168.z.11.i; A.169.z.11.i; A.170.z.11.i; A.171.z.11.i; A.172.z.11.i;
A.173.z.11.i; A.174.z.11.i; A.175.z.11.i; A.176.z.11.i; A.177.z.11.i; A.178.z.11.i;
A.179.z.11.i; A.180.z.11.i; A.181.z.11.i; A.182.z.11.i; A.183.z.11.i; A.184.z.11.i;
A.185.z.11.i; A.186.z.11.i; A.187.z.11.i; A.188.z.11.i; A.189.z.11.i; A.190.z.11.i;
A.191.z.11.i; A.192.z.11.i; A.193.z.11.i; A.194.z.11.i; A.195.z.11.i; A.196.z.11.i;
A.197.z.11.i; A.198.z.11.i; A.199.z.11.i; A.200.z.11.i; A.201.z.11.i; A.202.z.11.i;
A.203.z.11.i; A.204.z.11.i; A.205.z.11.i; A.206.z.11.i; A.207.z.11.i; A.208.z.11.i;
A.209.z.11.i; A.210.z.11.i; A.211.z.11.i; A.212.z.11.i; A.213.z.11.i; A.214.z.11.i;
A.215.z.11.i; A.216.z.11.i; A.217.z.11.i; A.218.z.11.i; A.219.z.11.i; A.220.z.11.i;
A.221.z.11.i; A.222.z.11.i; A.223.z.11.i; A.224.z.11.i; A.225.z.11.i; A.226.z.11.i;
A.227.z.11.i; A.228.z.11.i; A.229.z.11.i; A.230.z.11.i; A.231.z.11.i; A.232.z.11.i;
A.233.z.11.i; A.234.z.11.i; A.235.z.11.i; A.236.z.11.i; A.237.z.11.i; A.238.z.11.i;
A.239.z.11.i; A.240.z.11.i; A.241.z.11.i; A.242.z.11.i; A.243.z.11.i; A.244.z.11.i;
A.245.z.11.i; A.246.z.11.i; A.247.z.11.i; A.248.z.11.i; A.249.z.11.i; A.250.z.11.i;
A.251.z.11.i; A.252.z.11.i; A.253.z.11.i; A.254.z.11.i; A.255.z.11.i; A.256.z.11.i;
A.257.z.11.i; A.258.z.11.i; A.259.z.11.i; A.260.z.11.i; A.261.z.11.i; A.262.z.11.i;
A.263.z.11.i; A.264.z.11.i; A.265.z.11.i; A.266.z.11.i; A.267.z.11.i; A.268.z.11.i;
A.269.z.11.i; A.270.z.11.i; A.271.z.11.i; A.272.z.11.i; A.273.z.11.i; A.274.z.11.i;
A.275.z.11.i; A.276.z.11.i; A.277.z.11.i; A.278.z.11.i; A.279.z.11.i; A.280.z.11.i;
A.281.z.11.i; A.282.z.11.i; A.283.z.11.i; A.284.z.11.i; A.285.z.11.i; A.286.z.11.i;
A.287.z.11.i; A.288.z.11.i; A.289.z.11.i; A.290.z.11.i; A.291.z.11.i; A.292.z.11.i;
A.293.z.11.i; A.294.z.11.i; A.295.z.11.i; A.296.z.11.i; A.297.z.11.i; A.298.z.11.i;
A.299.z.11.i; A.300.z.11.i; A.301.z.11.i; A.302.z.11.i; A.303.z.11.i; A.304.z.11.i;
A.305.z.11.i; A.306.z.11.i; A.307.z.11.i; A.308.z.11.i; A.309.z.11.i; A.310.z.11.i;
A.311.z.11.i; A.312.z.11.i; A.313.z.11.i; A.314.z.11.i; A.315.z.11.i; A.316.z.11.i;
A.317.z.11.i; A.318.z.11.i; A.319.z.11.i; A.320.z.11.i; A.321.z.11.i; A.323.z.11.i;
A.324.z.11.i; A.325.z.11.i; A.326.z.11.i; A.327.z.11.i; A.328.z.11.i; A.329.z.11.i;
A.330.z.11.i; A.331.z.11.i; A.332.z.11.i; A.333.z.11.i; A.334.z.11.i; A.335.z.11.i;
A.336.z.11.i; A.337.z.11.i; A.338.z.11.i; A.339.z.11.i; A.340.z.11.i; A.341.z.11.i;
A.342.z.11.i; A.343.z.11.i; A.344.z.11.i; A.345.z.11.i; A.346.z.11.i; A.347.z.11.i;
A.348.z.11.i; A.349.z.11.i; A.350.z.11.i; A.351.z.11.i; A.352.z.11.i; A.353.z.11.i;
A.354.z.11.i; A.355.z.11.i; A.356.z.11.i; A.357.z.11.i; A.358.z.11.i; A.359.z.11.i;
A.360.z.11.i; A.361.z.11.i; A.362.z.11.i; A.363.z.11.i; A.364.z.11.i; A.365.z.11.i;
A.366.z.11.i; A.367.z.11.i; A.368.z.11.i; A.369.z.11.i; A.370.z.11.i; A.371.z.11.i;
A.372.z.11.i; A.373.z.11.i; A.374.z.11.i; A.375.z.11.i; A.376.z.11.i; A.377.z.11.i;
A.378.z.11.i; A.379.z.11.i; A.380.z.11.i; A.381.z.11.i; A.382.z.11.i; A.383.z.11.i;
A.384.z.11.i; A.385.z.11.i; A.386.z.11.i; A.387.z.11.i; A.388.z.11.i; A.389.z.11.i;
A.390.z.11.i; A.391.z.11.i; A.392.z.11.i; A.393.z.11.i; A.394.z.11.i; A.395.z.11.i;
A.396.z.11.i; A.397.z.11.i; A.398.z.11.i; A.399.z.11.i; A.400.z.11.i; A.401.z.11.i;
A.402.z.11.i; A.403.z.11.i; A.404.z.11.i; A.405.z.11.i; A.406.z.11.i; A.407.z.11.i;
A.408.z.11.i; A.409.z.11.i; A.410.z.11.i; A.411.z.11.i; A.412.z.11.i; A.413.z.11.i;
A.414.z.11.i; A.415.z.11.i; A.416.z.11.i; A.417.z.11.i; A.418.z.11.i; A.419.z.11.i;
A.420.z.11.i; A.421.z.11.i; A.422.z.11.i; A.423.z.11.i; A.424.z.11.i; A.425.z.11.i;
A.426.z.11.i; A.427.z.11.i; A.428.z.11.i; A.429.z.11.i; A.430.z.11.i; A.431.z.11.i;
A.432.z.11.i; A.433.z.11.i; A.434.z.11.i; A.435.z.11.i; A.436.z.11.i; A.437.z.11.i;
A.438.z.11.i; A.439.z.11.i; A.440.z.11.i; A.441.z.11.i; A.442.z.11.i; A.443.z.11.i;
A.444.z.11.i; A.445.z.11.i; A.446.z.11.i; A.447.z.11.i; A.448.z.11.i; A.449.z.11.i;
A.450.z.11.i; A.451.z.11.i; A.452.z.11.i; A.453.z.11.i; A.454.z.11.i; A.455.z.11.i;
A.456.z.11.i; A.457.z.11.i; A.458.z.11.i; A.459.z.11.i; A.460.z.11.i; A.461.z.11.i;
A.462.z.11.i; A.463.z.11.i; A.464.z.11.i; A.465.z.11.i; A.466.z.11.i; A.467.z.11.i;
A.468.z.11.i; A.469.z.11.i; A.470.z.11.i; A.471.z.11.i; A.472.z.11.i; A.473.z.11.i;
A.474.z.11.i; A.475.z.11.i; A.476.z.11.i; A.477.z.11.i; A.478.z.11.i; A.479.z.11.i;
A.480.z.11.i; A.481.z.11.i; A.482.z.11.i; A.483.z.11.i; A.484.z.11.i; A.485.z.11.i;
A.486.z.11.i; A.487.z.11.i; A.488.z.11.i; A.489.z.11.i; A.490.z.11.i; A.491.z.11.i;
A.492.z.11.i; A.493.z.11.i; A.494.z.11.i; A.495.z.11.i; A.496.z.11.i; A.497.z.11.i;
A.498.z.11.i; A.499.z.11.i; A.500.z.11.i; A.501.z.11.i; A.502.z.11.i; A.503.z.11.i;
A.504.z.11.i; A.505.z.11.i; A.506.z.11.i; A.507.z.11.i; A.508.z.11.i; A.509.z.11.i;
A.510.z.11.i; A.511.z.11.i; A.512.z.11.i; A.512.z.11.i; A.513.z.11.i; A.514.z.11.i;
A.515.z.11.i; A.516.z.11.i; A.517.z.11.i; A.518.z.11.i; A.519.z.11.i; A.520.z.11.i;
A.521.z.11.i; A.522.z.11.i; A.523.z.11.i; A.524.z.11.i; A.525.z.11.i; A.526.z.11.i;
A.527.z.11.i; A.528.z.11.i; A.529.z.11.i; A.530.z.11.i; A.531.z.11.i; A.532.z.11.i;
A.533.z.11.i; A.534.z.11.i; A.535.z.11.i; A.536.z.11.i; A.537.z.11.i; A.538.z.11.i;
A.539.z.11.i; A.540.z.11.i; A.541.z.11.i; A.542.z.11.i; A.543.z.11.i; A.544.z.11.i;
A.545.z.11.i; A.546.z.11.i; A.547.z.11.i; A.548.z.11.i; A.549.z.11.i; A.550.z.11.i;
A.551.z.11.i; A.552.z.11.i; A.553.z.11.i; A.554.z.11.i; A.555.z.11.i; A.556.z.11.i;
A.557.z.11.i; A.558.z.11.i; A.559.z.11.i; A.560.z.11.i; A.561.z.11.i; A.562.z.11.i;
A.563.z.11.i; A.564.z.11.i; A.565.z.11.i; A.566.z.11.i; A.567.z.11.i; A.568.z.11.i;
A.569.z.11.i; A.570.z.11.i; A.571.z.11.i; A.572.z.11.i; A.573.z.11.i; A.574.z.11.i;
A.575.z.11.i; A.576.z.11.i; A.577.z.11.i; A.578.z.11.i; A.579.z.11.i; A.580.z.11.i;
A.581.z.11.i; A.582.z.11.i; A.583.z.11.i; A.584.z.11.i; A.585.z.11.i; A.586.z.11.i;
A.587.z.11.i; A.588.z.11.i; A.589.z.11.i; A.590.z.11.i; A.591.z.11.i; A.592.z.11.i;
A.593.z.11.i; A.594.z.11.i; A.595.z.11.i; A.596.z.11.i; A.597.z.11.i; A.598.z.11.i;
A.599.z.11.i; A.600.z.11.i; A.601.z.11.i; A.602.z.11.i; A.603.z.11.i; A.604.z.11.i;
A.605.z.11.i; A.606.z.11.i; A.607.z.11.i; A.608.z.11.i; A.609.z.11.i; A.610.z.11.i;
A.611.z.11.i; A.612.z.11.i; A.613.z.11.i; A.614.z.11.i; A.615.z.11.i; A.616.z.11.i;
A.617.z.11.i; A.618.z.11.i; A.619.z.11.i; A.620.z.11.i; A.621.z.11.i; A.622.z.11.i;
A.623.z.11.i; A.624.z.11.i; A.625.z.11.i; A.626.z.11.i; A.627.z.11.i; A.628.z.11.i;
A.629.z.11.i; A.630.z.11.i; A.631.z.11.i; A.632.z.11.i; A.633.z.11.i; A.634.z.11.i;
A.635.z.11.i; A.636.z.11.i; A.637.z.11.i; A.638.z.11.i; A.639.z.11.i; A.640.z.11.i;
A.641.z.11.i; A.642.z.11.i; A.643.z.11.i; A.644.z.11.i; A.645.z.11.i; A.646.z.11.i;
A.647.z.11.i; A.648.z.11.i; A.649.z.11.i; A.650.z.11.i; A.651.z.11.i; A.652.z.11.i;
A.653.z.11.i; A.654.z.11.i; A.655.z.11.i; A.656.z.11.i; A.657.z.11.i; A.658.z.11.i;
A.659.z.11.i; A.660.z.11.i; A.2.A.4.i; A.3.A.4.i; A.4.A.4.i; A.5.A.4.i; A.6.A.4.i;
A.7.A.4.i; A.9.A.4.i; A.10.A.4.i; A.15.A.4.i; A.100.A.4.i; A.101.A.4.i; A.102.A.4.i;
A.103.A.4.i; A.104.A.4.i; A.105.A.4.i; A.106.A.4.i; A.107.A.4.i; A.108.A.4.i;
A.109.A.4.i; A.110.A.4.i; A.111.A.4.i; A.112.A.4.i; A.113.A.4.i; A.114.A.4.i;
A.115.A.4.i; A.116.A.4.i; A.117.A.4.i; A.118.A.4.i; A.119.A.4.i; A.120.A.4.i;
A.121.A.4.i; A.122.A.4.i; A.123.A.4.i; A.124.A.4.i; A.125.A.4.i; A.126.A.4.i;
A.127.A.4.i; A.128.A.4.i; A.129.A.4.i; A.130.A.4.i; A.131.A.4.i; A.132.A.4.i;
A.133.A.4.i; A.134.A.4.i; A.135.A.4.i; A.136.A.4.i; A.137.A.4.i; A.138.A.4.i;
A.139.A.4.i; A.140.A.4.i; A.141.A.4.i; A.142.A.4.i; A.143.A.4.i; A.144.A.4.i;
A.145.A.4.i; A.146.A.4.i; A.147.A.4.i; A.148.A.4.i; A.149.A.4.i; A.150.A.4.i;
A.151.A.4.i; A.152.A.4.i; A.153.A.4.i; A.154.A.4.i; A.155.A.4.i; A.156.A.4.i;
A.157.A.4.i; A.158.A.4.i; A.159.A.4.i; A.160.A.4.i; A.161.A.4.i; A.162.A.4.i;
A.163.A.4.i; A.164.A.4.i; A.165.A.4.i; A.166.A.4.i; A.167.A.4.i; A.168.A.4.i;
A.169.A.4.i; A.170.A.4.i; A.171.A.4.i; A.172.A.4.i; A.173.A.4.i; A.174.A.4.i;
A.175.A.4.i; A.176.A.4.i; A.177.A.4.i; A.178.A.4.i; A.179.A.4.i; A.180.A.4.i;
A.181.A.4.i; A.182.A.4.i; A.183.A.4.i; A.184.A.4.i; A.185.A.4.i; A.186.A.4.i;
A.187.A.4.i; A.188.A.4.i; A.189.A.4.i; A.190.A.4.i; A.191.A.4.i; A.192.A.4.i;
A.193.A.4.i; A.194.A.4.i; A.195.A.4.i; A.196.A.4.i; A.197.A.4.i; A.198.A.4.i;
A.199.A.4.i; A.200.A.4.i; A.201.A.4.i; A.202.A.4.i; A.203.A.4.i; A.204.A.4.i;
A.205.A.4.i; A.206.A.4.i; A.207.A.4.i; A.208.A.4.i; A.209.A.4.i; A.210.A.4.i;
A.211.A.4.i; A.212.A.4.i; A.213.A.4.i; A.214.A.4.i; A.215.A.4.i; A.216.A.4.i;
A.217.A.4.i; A.218.A.4.i; A.219.A.4.i; A.220.A.4.i; A.221.A.4.i; A.222.A.4.i;
A.223.A.4.i; A.224.A.4.i; A.225.A.4.i; A.226.A.4.i; A.227.A.4.i; A.228.A.4.i;
A.229.A.4.i; A.230.A.4.i; A.231.A.4.i; A.232.A.4.i; A.233.A.4.i; A.234.A.4.i;
A.235.A.4.i; A.236.A.4.i; A.237.A.4.i; A.238.A.4.i; A.239.A.4.i; A.240.A.4.i;
A.241.A.4.i; A.242.A.4.i; A.243.A.4.i; A.244.A.4.i; A.245.A.4.i; A.246.A.4.i;
A.247.A.4.i; A.248.A.4.i; A.249.A.4.i; A.250.A.4.i; A.251.A.4.i; A.252.A.4.i;
A.253.A.4.i; A.254.A.4.i; A.255.A.4.i; A.256.A.4.i; A.257.A.4.i; A.258.A.4.i;
A.259.A.4.i; A.260.A.4.i; A.261.A.4.i; A.262.A.4.i; A.263.A.4.i; A.264.A.4.i;
A.265.A.4.i; A.266.A.4.i; A.267.A.4.i; A.268.A.4.i; A.269.A.4.i; A.270.A.4.i;
A.271.A.4.i; A.272.A.4.i; A.273.A.4.i; A.274.A.4.i; A.275.A.4.i; A.276.A.4.i;
A.277.A.4.i; A.278.A.4.i; A.279.A.4.i; A.280.A.4.i; A.281.A.4.i; A.282.A.4.i;
A.283.A.4.i; A.284.A.4.i; A.285.A.4.i; A.286.A.4.i; A.287.A.4.i; A.288.A.4.i;
A.289.A.4.i; A.290.A.4.i; A.291.A.4.i; A.292.A.4.i; A.293.A.4.i; A.294.A.4.i;
A.295.A.4.i; A.296.A.4.i; A.297.A.4.i; A.298.A.4.i; A.299.A.4.i; A.300.A.4.i;
A.301.A.4.i; A.302.A.4.i; A.303.A.4.i; A.304.A.4.i; A.305.A.4.i; A.306.A.4.i;
A.307.A.4.i; A.308.A.4.i; A.309.A.4.i; A.310.A.4.i; A.311.A.4.i; A.312.A.4.i;
A.313.A.4.i; A.314.A.4.i; A.315.A.4.i; A.316.A.4.i; A.317.A.4.i; A.318.A.4.i;
A.319.A.4.i; A.320.A.4.i; A.321.A.4.i; A.323.A.4.i; A.324.A.4.i; A.325.A.4.i;
A.326.A.4.i; A.327.A.4.i; A.328.A.4.i; A.329.A.4.i; A.330.A.4.i; A.331.A.4.i;
A.332.A.4.i; A.333.A.4.i; A.334.A.4.i; A.335.A.4.i; A.336.A.4.i; A.337.A.4.i;
A.338.A.4.i; A.339.A.4.i; A.340.A.4.i; A.341.A.4.i; A.342.A.4.i; A.343.A.4.i;
A.344.A.4.i; A.345.A.4.i; A.346.A.4.i; A.347.A.4.i; A.348.A.4.i; A.349.A.4.i;
A.350.A.4.i; A.351.A.4.i; A.352.A.4.i; A.353.A.4.i; A.354.A.4.i; A.355.A.4.i;
A.356.A.4.i; A.357.A.4.i; A.358.A.4.i; A.359.A.4.i; A.360.A.4.i; A.361.A.4.i;
A.362.A.4.i; A.363.A.4.i; A.364.A.4.i; A.365.A.4.i; A.366.A.4.i; A.367.A.4.i;
A.368.A.4.i; A.369.A.4.i; A.370.A.4.i; A.371.A.4.i; A.372.A.4.i; A.373.A.4.i;
A.374.A.4.i; A.375.A.4.i; A.376.A.4.i; A.377.A.4.i; A.378.A.4.i; A.379.A.4.i;
A.380.A.4.i; A.381.A.4.i; A.382.A.4.i; A.383.A.4.i; A.384.A.4.i; A.385.A.4.i;
A.386.A.4.i; A.387.A.4.i; A.388.A.4.i; A.389.A.4.i; A.390.A.4.i; A.391.A.4.i;
A.392.A.4.i; A.393.A.4.i; A.394.A.4.i; A.395.A.4.i; A.396.A.4.i; A.397.A.4.i;
A.398.A.4.i; A.399.A.4.i; A.400.A.4.i; A.401.A.4.i; A.402.A.4.i; A.403.A.4.i;
A.404.A.4.i; A.405.A.4.i; A.406.A.4.i; A.407.A.4.i; A.408.A.4.i; A.409.A.4.i;
A.410.A.4.i; A.411.A.4.i; A.412.A.4.i; A.413.A.4.i; A.414.A.4.i; A.415.A.4.i;
A.416.A.4.i; A.417.A.4.i; A.418.A.4.i; A.419.A.4.i; A.420.A.4.i; A.421.A.4.i;
A.422.A.4.i; A.423.A.4.i; A.424.A.4.i; A.425.A.4.i; A.426.A.4.i; A.427.A.4.i;
A.428.A.4.i; A.429.A.4.i; A.430.A.4.i; A.431.A.4.i; A.432.A.4.i; A.433.A.4.i;
A.434.A.4.i; A.435.A.4.i; A.436.A.4.i; A.437.A.4.i; A.438.A.4.i; A.439.A.4.i;
A.440.A.4.i; A.441.A.4.i; A.442.A.4.i; A.443.A.4.i; A.444.A.4.i; A.445.A.4.i;
A.446.A.4.i; A.447.A.4.i; A.448.A.4.i; A.449.A.4.i; A.450.A.4.i; A.451.A.4.i;
A.452.A.4.i; A.453.A.4.i; A.454.A.4.i; A.455.A.4.i; A.456.A.4.i; A.457.A.4.i;
A.458.A.4.i; A.459.A.4.i; A.460.A.4.i; A.461.A.4.i; A.462.A.4.i; A.463.A.4.i;
A.464.A.4.i; A.465.A.4.i; A.466.A.4.i; A.467.A.4.i; A.468.A.4.i; A.469.A.4.i;
A.470.A.4.i; A.471.A.4.i; A.472.A.4.i; A.473.A.4.i; A.474.A.4.i; A.475.A.4.i;
A.476.A.4.i; A.477.A.4.i; A.478.A.4.i; A.479.A.4.i; A.480.A.4.i; A.481.A.4.i;
A.482.A.4.i; A.483.A.4.i; A.484.A.4.i; A.485.A.4.i; A.486.A.4.i; A.487.A.4.i;
A.488.A.4.i; A.489.A.4.i; A.490.A.4.i; A.491.A.4.i; A.492.A.4.i; A.493.A.4.i;
A.494.A.4.i; A.495.A.4.i; A.496.A.4.i; A.497.A.4.i; A.498.A.4.i; A.499.A.4.i;
A.500.A.4.i; A.501.A.4.i; A.502.A.4.i; A.503.A.4.i; A.504.A.4.i; A.505.A.4.i;
A.506.A.4.i; A.507.A.4.i; A.508.A.4.i; A.509.A.4.i; A.510.A.4.i; A.511.A.4.i;
A.512.A.4.i; A.512.A.4.i; A.513.A.4.i; A.514.A.4.i; A.515.A.4.i; A.516.A.4.i;
A.517.A.4.i; A.518.A.4.i; A.519.A.4.i; A.520.A.4.i; A.521.A.4.i; A.522.A.4.i;
A.523.A.4.i; A.524.A.4.i; A.525.A.4.i; A.526.A.4.i; A.527.A.4.i; A.528.A.4.i;
A.529.A.4.i; A.530.A.4.i; A.531.A.4.i; A.532.A.4.i; A.533.A.4.i; A.534.A.4.i;
A.535.A.4.i; A.536.A.4.i; A.537.A.4.i; A.538.A.4.i; A.539.A.4.i; A.540.A.4.i;
A.541.A.4.i; A.542.A.4.i; A.543.A.4.i; A.544.A.4.i; A.545.A.4.i; A.546.A.4.i;
A.547.A.4.i; A.548.A.4.i; A.549.A.4.i; A.550.A.4.i; A.551.A.4.i; A.552.A.4.i;
A.553.A.4.i; A.554.A.4.i; A.555.A.4.i; A.556.A.4.i; A.557.A.4.i; A.558.A.4.i;
A.559.A.4.i; A.560.A.4.i; A.561.A.4.i; A.562.A.4.i; A.563.A.4.i; A.564.A.4.i;
A.565.A.4.i; A.566.A.4.i; A.567.A.4.i; A.568.A.4.i; A.569.A.4.i; A.570.A.4.i;
A.571.A.4.i; A.572.A.4.i; A.573.A.4.i; A.574.A.4.i; A.575.A.4.i; A.576.A.4.i;
A.577.A.4.i; A.578.A.4.i; A.579.A.4.i; A.580.A.4.i; A.581.A.4.i; A.582.A.4.i;
A.583.A.4.i; A.584.A.4.i; A.585.A.4.i; A.586.A.4.i; A.587.A.4.i; A.588.A.4.i;
A.589.A.4.i; A.590.A.4.i; A.591.A.4.i; A.592.A.4.i; A.593.A.4.i; A.594.A.4.i;
A.595.A.4.i; A.596.A.4.i; A.597.A.4.i; A.598.A.4.i; A.599.A.4.i; A.600.A.4.i;
A.601.A.4.i; A.602.A.4.i; A.603.A.4.i; A.604.A.4.i; A.605.A.4.i; A.606.A.4.i;
A.607.A.4.i; A.608.A.4.i; A.609.A.4.i; A.610.A.4.i; A.611.A.4.i; A.612.A.4.i;
A.613.A.4.i; A.614.A.4.i; A.615.A.4.i; A.616.A.4.i; A.617.A.4.i; A.618.A.4.i;
A.619.A.4.i; A.620.A.4.i; A.621.A.4.i; A.622.A.4.i; A.623.A.4.i; A.624.A.4.i;
A.625.A.4.i; A.626.A.4.i; A.627.A.4.i; A.628.A.4.i; A.629.A.4.i; A.630.A.4.i;
A.631.A.4.i; A.632.A.4.i; A.633.A.4.i; A.634.A.4.i; A.635.A.4.i; A.636.A.4.i;
A.637.A.4.i; A.638.A.4.i; A.639.A.4.i; A.640.A.4.i; A.641.A.4.i; A.642.A.4.i;
A.643.A.4.i; A.644.A.4.i; A.645.A.4.i; A.646.A.4.i; A.647.A.4.i; A.648.A.4.i;
A.649.A.4.i; A.650.A.4.i; A.651.A.4.i; A.652.A.4.i; A.653.A.4.i; A.654.A.4.i;
A.655.A.4.i; A.656.A.4.i; A.657.A.4.i; A.658.A.4.i; A.659.A.4.i; A.660.A.4.i;
A.2.A.11.i; A.3.A.11.i; A.4.A.11.i; A.5.A.11.i; A.6.A.11.i; A.7.A.11.i; A.9.A.11.i;
A.10.A.11.i; A.15.A.11.i; A.100.A.11.i; A.101.A.11.i; A.102.A.11.i; A.103.A.11.i;
A.104.A.11.i; A.105.A.11.i; A.106.A.11.i; A.107.A.11.i; A.108.A.11.i; A.109.A.11.i;
A.110.A.11.i; A.111.A.11.i; A.112.A.11.i; A.113.A.11.i; A.114.A.11.i; A.115.A.11.i;
A.116.A.11.i; A.117.A.11.i; A.118.A.11.i; A.119.A.11.i; A.120.A.11.i; A.121.A.11.i;
A.122.A.11.i; A.123.A.11.i; A.124.A.11.i; A.125.A.11.i; A.126.A.11.i; A.127.A.11.i;
A.128.A.11.i; A.129.A.11.i; A.130.A.11.i; A.131.A.11.i; A.132.A.11.i; A.133.A.11.i;
A.134.A.11.i; A.135.A.11.i; A.136.A.11.i; A.137.A.11.i; A.138.A.11.i; A.139.A.11.i;
A.140.A.11.i; A.141.A.11.i; A.142.A.11.i; A.143.A.11.i; A.144.A.11.i; A.145.A.11.i;
A.146.A.11.i; A.147.A.11.i; A.148.A.11.i; A.149.A.11.i; A.150.A.11.i; A.151.A.11.i;
A.152.A.11.i; A.153.A.11.i; A.154.A.11.i; A.155.A.11.i; A.156.A.11.i; A.157.A.11.i;
A.158.A.11.i; A.159.A.11.i; A.160.A.11.i; A.161.A.11.i; A.162.A.11.i; A.163.A.11.i;
A.164.A.11.i; A.165.A.11.i; A.166.A.11.i; A.167.A.11.i; A.168.A.11.i; A.169.A.11.i;
A.170.A.11.i; A.171.A.11.i; A.172.A.11.i; A.173.A.11.i; A.174.A.11.i; A.175.A.11.i;
A.176.A.11.i; A.177.A.11.i; A.178.A.11.i; A.179.A.11.i; A.180.A.11.i; A.181.A.11.i;
A.182.A.11.i; A.183.A.11.i; A.184.A.11.i; A.185.A.11.i; A.186.A.11.i; A.187.A.11.i;
A.188.A.11.i; A.189.A.11.i; A.190.A.11.i; A.191.A.11.i; A.192.A.11.i; A.193.A.11.i;
A.194.A.11.i; A.195.A.11.i; A.196.A.11.i; A.197.A.11.i; A.198.A.11.i; A.199.A.11.i;
A.200.A.11.i; A.201.A.11.i; A.202.A.11.i; A.203.A.11.i; A.204.A.11.i; A.205.A.11.i;
A.206.A.11.i; A.207.A.11.i; A.208.A.11.i; A.209.A.11.i; A.210.A.11.i; A.211.A.11.i;
A.212.A.11.i; A.213.A.11.i; A.214.A.11.i; A.215.A.11.i; A.216.A.11.i; A.217.A.11.i;
A.218.A.11.i; A.219.A.11.i; A.220.A.11.i; A.221.A.11.i; A.222.A.11.i; A.223.A.11.i;
A.224.A.11.i; A.225.A.11.i; A.226.A.11.i; A.227.A.11.i; A.228.A.11.i; A.229.A.11.i;
A.230.A.11.i; A.231.A.11.i; A.232.A.11.i; A.233.A.11.i; A.234.A.11.i; A.235.A.11.i;
A.236.A.11.i; A.237.A.11.i; A.238.A.11.i; A.239.A.11.i; A.240.A.11.i; A.241.A.11.i;
A.242.A.11.i; A.243.A.11.i; A.244.A.11.i; A.245.A.11.i; A.246.A.11.i; A.247.A.11.i;
A.248.A.11.i; A.249.A.11.i; A.250.A.11.i; A.251.A.11.i; A.252.A.11.i; A.253.A.11.i;
A.254.A.11.i; A.255.A.11.i; A.256.A.11.i; A.257.A.11.i; A.258.A.11.i; A.259.A.11.i;
A.260.A.11.i; A.261.A.11.i; A.262.A.11.i; A.263.A.11.i; A.264.A.11.i; A.265.A.11.i;
A.266.A.11.i; A.267.A.11.i; A.268.A.11.i; A.269.A.11.i; A.270.A.11.i; A.271.A.11.i;
A.272.A.11.i; A.273.A.11.i; A.274.A.11.i; A.275.A.11.i; A.276.A.11.i; A.277.A.11.i;
A.278.A.11.i; A.279.A.11.i; A.280.A.11.i; A.281.A.11.i; A.282.A.11.i; A.283.A.11.i;
A.284.A.11.i; A.285.A.11.i; A.286.A.11.i; A.287.A.11.i; A.288.A.11.i; A.289.A.11.i;
A.290.A.11.i; A.291.A.11.i; A.292.A.11.i; A.293.A.11.i; A.294.A.11.i; A.295.A.11.i;
A.296.A.11.i; A.297.A.11.i; A.298.A.11.i; A.299.A.11.i; A.300.A.11.i; A.301.A.11.i;
A.302.A.11.i; A.303.A.11.i; A.304.A.11.i; A.305.A.11.i; A.306.A.11.i; A.307.A.11.i;
A.308.A.11.i; A.309.A.11.i; A.310.A.11.i; A.311.A.11.i; A.312.A.11.i; A.313.A.11.i;
A.314.A.11.i; A.315.A.11.i; A.316.A.11.i; A.317.A.11.i; A.318.A.11.i; A.319.A.11.i;
A.320.A.11.i; A.321.A.11.i; A.323.A.11.i; A.324.A.11.i; A.325.A.11.i; A.326.A.11.i;
A.327.A.11.i; A.328.A.11.i; A.329.A.11.i; A.330.A.11.i; A.331.A.11.i; A.332.A.11.i;
A.333.A.11.i; A.334.A.11.i; A.335.A.11.i; A.336.A.11.i; A.337.A.11.i; A.338.A.11.i;
A.339.A.11.i; A.340.A.11.i; A.341.A.11.i; A.342.A.11.i; A.343.A.11.i; A.344.A.11.i;
A.345.A.11.i; A.346.A.11.i; A.347.A.11.i; A.348.A.11.i; A.349.A.11.i; A.350.A.11.i;
A.351.A.11.i; A.352.A.11.i; A.353.A.11.i; A.354.A.11.i; A.355.A.11.i; A.356.A.11.i;
A.357.A.11.i; A.358.A.11.i; A.359.A.11.i; A.360.A.11.i; A.361.A.11.i; A.362.A.11.i;
A.363.A.11.i; A.364.A.11.i; A.365.A.11.i; A.366.A.11.i; A.367.A.11.i; A.368.A.11.i;
A.369.A.11.i; A.370.A.11.i; A.371.A.11.i; A.372.A.11.i; A.373.A.11.i; A.374.A.11.i;
A.375.A.11.i; A.376.A.11.i; A.377.A.11.i; A.378.A.11.i; A.379.A.11.i; A.380.A.11.i;
A.381.A.11.i; A.382.A.11.i; A.383.A.11.i; A.384.A.11.i; A.385.A.11.i; A.386.A.11.i;
A.387.A.11.i; A.388.A.11.i; A.389.A.11.i; A.390.A.11.i; A.391.A.11.i; A.392.A.11.i;
A.393.A.11.i; A.394.A.11.i; A.395.A.11.i; A.396.A.11.i; A.397.A.11.i; A.398.A.11.i;
A.399.A.11.i; A.400.A.11.i; A.401.A.11.i; A.402.A.11.i; A.403.A.11.i; A.404.A.11.i;
A.405.A.11.i; A.406.A.11.i; A.407.A.11.i; A.408.A.11.i; A.409.A.11.i; A.410.A.11.i;
A.411.A.11.i; A.412.A.11.i; A.413.A.11.i; A.414.A.11.i; A.415.A.11.i; A.416.A.11.i;
A.417.A.11.i; A.418.A.11.i; A.419.A.11.i; A.420.A.11.i; A.421.A.11.i; A.422.A.11.i;
A.423.A.11.i; A.424.A.11.i; A.425.A.11.i; A.426.A.11.i; A.427.A.11.i; A.428.A.11.i;
A.429.A.11.i; A.430.A.11.i; A.431.A.11.i; A.432.A.11.i; A.433.A.11.i; A.434.A.11.i;
A.435.A.11.i; A.436.A.11.i; A.437.A.11.i; A.438.A.11.i; A.439.A.11.i; A.440.A.11.i;
A.441.A.11.i; A.442.A.11.i; A.443.A.11.i; A.444.A.11.i; A.445.A.11.i; A.446.A.11.i;
A.447.A.11.i; A.448.A.11.i; A.449.A.11.i; A.450.A.11.i; A.451.A.11.i; A.452.A.11.i;
A.453.A.11.i; A.454.A.11.i; A.455.A.11.i; A.456.A.11.i; A.457.A.11.i; A.458.A.11.i;
A.459.A.11.i; A.460.A.11.i; A.461.A.11.i; A.462.A.11.i; A.463.A.11.i; A.464.A.11.i;
A.465.A.11.i; A.466.A.11.i; A.467.A.11.i; A.468.A.11.i; A.469.A.11.i; A.470.A.11.i;
A.471.A.11.i; A.472.A.11.i; A.473.A.11.i; A.474.A.11.i; A.475.A.11.i; A.476.A.11.i;
A.477.A.11.i; A.478.A.11.i; A.479.A.11.i; A.480.A.11.i; A.481.A.11.i; A.482.A.11.i;
A.483.A.11.i; A.484.A.11.i; A.485.A.11.i; A.486.A.11.i; A.487.A.11.i; A.488.A.11.i;
A.489.A.11.i; A.490.A.11.i; A.491.A.11.i; A.492.A.11.i; A.493.A.11.i; A.494.A.11.i;
A.495.A.11.i; A.496.A.11.i; A.497.A.11.i; A.498.A.11.i; A.499.A.11.i; A.500.A.11.i;
A.501.A.11.i; A.502.A.11.i; A.503.A.11.i; A.504.A.11.i; A.505.A.11.i; A.506.A.11.i;
A.507.A.11.i; A.508.A.11.i; A.509.A.11.i; A.510.A.11.i; A.511.A.11.i; A.512.A.11.i;
A.512.A.11.i; A.513.A.11.i; A.514.A.11.i; A.515.A.11.i; A.516.A.11.i; A.517.A.11.i;
A.518.A.11.i; A.519.A.11.i; A.520.A.11.i; A.521.A.11.i; A.522.A.11.i; A.523.A.11.i;
A.524.A.11.i; A.525.A.11.i; A.526.A.11.i; A.527.A.11.i; A.528.A.11.i; A.529.A.11.i;
A.530.A.11.i; A.531.A.11.i; A.532.A.11.i; A.533.A.11.i; A.534.A.11.i; A.535.A.11.i;
A.536.A.11.i; A.537.A.11.i; A.538.A.11.i; A.539.A.11.i; A.540.A.11.i; A.541.A.11.i;
A.542.A.11.i; A.543.A.11.i; A.544.A.11.i; A.545.A.11.i; A.546.A.11.i; A.547.A.11.i;
A.548.A.11.i; A.549.A.11.i; A.550.A.11.i; A.551.A.11.i; A.552.A.11.i; A.553.A.11.i;
A.554.A.11.i; A.555.A.11.i; A.556.A.11.i; A.557.A.11.i; A.558.A.11.i; A.559.A.11.i;
A.560.A.11.i; A.561.A.11.i; A.562.A.11.i; A.563.A.11.i; A.564.A.11.i; A.565.A.11.i;
A.566.A.11.i; A.567.A.11.i; A.568.A.11.i; A.569.A.11.i; A.570.A.11.i; A.571.A.11.i;
A.572.A.11.i; A.573.A.11.i; A.574.A.11.i; A.575.A.11.i; A.576.A.11.i; A.577.A.11.i;
A.578.A.11.i; A.579.A.11.i; A.580.A.11.i; A.581.A.11.i; A.582.A.11.i; A.583.A.11.i;
A.584.A.11.i; A.585.A.11.i; A.586.A.11.i; A.587.A.11.i; A.588.A.11.i; A.589.A.11.i;
A.590.A.11.i; A.591.A.11.i; A.592.A.11.i; A.593.A.11.i; A.594.A.11.i; A.595.A.11.i;
A.596.A.11.i; A.597.A.11.i; A.598.A.11.i; A.599.A.11.i; A.600.A.11.i; A.601.A.11.i;
A.602.A.11.i; A.603.A.11.i; A.604.A.11.i; A.605.A.11.i; A.606.A.11.i; A.607.A.11.i;
A.608.A.11.i; A.609.A.11.i; A.610.A.11.i; A.611.A.11.i; A.612.A.11.i; A.613.A.11.i;
A.614.A.11.i; A.615.A.11.i; A.616.A.11.i; A.617.A.11.i; A.618.A.11.i; A.619.A.11.i;
A.620.A.11.i; A.621.A.11.i; A.622.A.11.i; A.623.A.11.i; A.624.A.11.i; A.625.A.11.i;
A.626.A.11.i; A.627.A.11.i; A.628.A.11.i; A.629.A.11.i; A.630.A.11.i; A.631.A.11.i;
A.632.A.11.i; A.633.A.11.i; A.634.A.11.i; A.635.A.11.i; A.636.A.11.i; A.637.A.11.i;
A.638.A.11.i; A.639.A.11.i; A.640.A.11.i; A.641.A.11.i; A.642.A.11.i; A.643.A.11.i;
A.644.A.11.i; A.645.A.11.i; A.646.A.11.i; A.647.A.11.i; A.648.A.11.i; A.649.A.11.i;
A.650.A.11.i; A.651.A.11.i; A.652.A.11.i; A.653.A.11.i; A.654.A.11.i; A.655.A.11.i;
A.656.A.11.i; A.657.A.11.i; A.658.A.11.i; A.659.A.11.i; A.660.A.11.i; A.2.B.4.i;
A.3.B.4.i; A.4.B.4.i; A.5.B.4.i; A.6.B.4.i; A.7.B.4.i; A.9.B.4.i; A.10.B.4.i; A.15.B.4.i;
A.100.B.4.i; A.101.B.4.i; A.102.B.4.i; A.103.B.4.i; A.104.B.4.i; A.105.B.4.i;
A.106.B.4.i; A.107.B.4.i; A.108.B.4.i; A.109.B.4.i; A.110.B.4.i; A.111.B.4.i;
A.112.B.4.i; A.113.B.4.i; A.114.B.4.i; A.115.B.4.i; A.116.B.4.i; A.117.B.4.i;
A.118.B.4.i; A.119.B.4.i; A.120.B.4.i; A.121.B.4.i; A.122.B.4.i; A.123.B.4.i;
A.124.B.4.i; A.125.B.4.i; A.126.B.4.i; A.127.B.4.i; A.128.B.4.i; A.129.B.4.i;
A.130.B.4.i; A.131.B.4.i; A.132.B.4.i; A.133.B.4.i; A.134.B.4.i; A.135.B.4.i;
A.136.B.4.i; A.137.B.4.i; A.138.B.4.i; A.139.B.4.i; A.140.B.4.i; A.141.B.4.i;
A.142.B.4.i; A.143.B.4.i; A.144.B.4.i; A.145.B.4.i; A.146.B.4.i; A.147.B.4.i;
A.148.B.4.i; A.149.B.4.i; A.150.B.4.i; A.151.B.4.i; A.152.B.4.i; A.153.B.4.i;
A.154.B.4.i; A.155.B.4.i; A.156.B.4.i; A.157.B.4.i; A.158.B.4.i; A.159.B.4.i;
A.160.B.4.i; A.161.B.4.i; A.162.B.4.i; A.163.B.4.i; A.164.B.4.i; A.165.B.4.i;
A.166.B.4.i; A.167.B.4.i; A.168.B.4.i; A.169.B.4.i; A.170.B.4.i; A.171.B.4.i;
A.172.B.4.i; A.173.B.4.i; A.174.B.4.i; A.175.B.4.i; A.176.B.4.i; A.177.B.4.i;
A.178.B.4.i; A.179.B.4.i; A.180.B.4.i; A.181.B.4.i; A.182.B.4.i; A.183.B.4.i;
A.184.B.4.i; A.185.B.4.i; A.186.B.4.i; A.187.B.4.i; A.188.B.4.i; A.189.B.4.i;
A.190.B.4.i; A.191.B.4.i; A.192.B.4.i; A.193.B.4.i; A.194.B.4.i; A.195.B.4.i;
A.196.B.4.i; A.197.B.4.i; A.198.B.4.i; A.199.B.4.i; A.200.B.4.i; A.201.B.4.i;
A.202.B.4.i; A.203.B.4.i; A.204.B.4.i; A.205.B.4.i; A.206.B.4.i; A.207.B.4.i;
A.208.B.4.i; A.209.B.4.i; A.210.B.4.i; A.211.B.4.i; A.212.B.4.i; A.213.B.4.i;
A.214.B.4.i; A.215.B.4.i; A.216.B.4.i; A.217.B.4.i; A.218.B.4.i; A.219.B.4.i;
A.220.B.4.i; A.221.B.4.i; A.222.B.4.i; A.223.B.4.i; A.224.B.4.i; A.225.B.4.i;
A.226.B.4.i; A.227.B.4.i; A.228.B.4.i; A.229.B.4.i; A.230.B.4.i; A.231.B.4.i;
A.232.B.4.i; A.233.B.4.i; A.234.B.4.i; A.235.B.4.i; A.236.B.4.i; A.237.B.4.i;
A.238.B.4.i; A.239.B.4.i; A.240.B.4.i; A.241.B.4.i; A.242.B.4.i; A.243.B.4.i;
A.244.B.4.i; A.245.B.4.i; A.246.B.4.i; A.247.B.4.i; A.248.B.4.i; A.249.B.4.i;
A.250.B.4.i; A.251.B.4.i; A.252.B.4.i; A.253.B.4.i; A.254.B.4.i; A.255.B.4.i;
A.256.B.4.i; A.257.B.4.i; A.258.B.4.i; A.259.B.4.i; A.260.B.4.i; A.261.B.4.i;
A.262.B.4.i; A.263.B.4.i; A.264.B.4.i; A.265.B.4.i; A.266.B.4.i; A.267.B.4.i;
A.268.B.4.i; A.269.B.4.i; A.270.B.4.i; A.271.B.4.i; A.272.B.4.i; A.273.B.4.i;
A.274.B.4.i; A.275.B.4.i; A.276.B.4.i; A.277.B.4.i; A.278.B.4.i; A.279.B.4.i;
A.280.B.4.i; A.281.B.4.i; A.282.B.4.i; A.283.B.4.i; A.284.B.4.i; A.285.B.4.i;
A.286.B.4.i; A.287.B.4.i; A.288.B.4.i; A.289.B.4.i; A.290.B.4.i; A.291.B.4.i;
A.292.B.4.i; A.293.B.4.i; A.294.B.4.i; A.295.B.4.i; A.296.B.4.i; A.297.B.4.i;
A.298.B.4.i; A.299.B.4.i; A.300.B.4.i; A.301.B.4.i; A.302.B.4.i; A.303.B.4.i;
A.304.B.4.i; A.305.B.4.i; A.306.B.4.i; A.307.B.4.i; A.308.B.4.i; A.309.B.4.i;
A.310.B.4.i; A.311.B.4.i; A.312.B.4.i; A.313.B.4.i; A.314.B.4.i; A.315.B.4.i;
A.316.B.4.i; A.317.B.4.i; A.318.B.4.i; A.319.B.4.i; A.320.B.4.i; A.321.B.4.i;
A.323.B.4.i; A.324.B.4.i; A.325.B.4.i; A.326.B.4.i; A.327.B.4.i; A.328.B.4.i;
A.329.B.4.i; A.330.B.4.i; A.331.B.4.i; A.332.B.4.i; A.333.B.4.i; A.334.B.4.i;
A.335.B.4.i; A.336.B.4.i; A.337.B.4.i; A.338.B.4.i; A.339.B.4.i; A.340.B.4.i;
A.341.B.4.i; A.342.B.4.i; A.343.B.4.i; A.344.B.4.i; A.345.B.4.i; A.346.B.4.i;
A.347.B.4.i; A.348.B.4.i; A.349.B.4.i; A.350.B.4.i; A.351.B.4.i; A.352.B.4.i;
A.353.B.4.i; A.354.B.4.i; A.355.B.4.i; A.356.B.4.i; A.357.B.4.i; A.358.B.4.i;
A.359.B.4.i; A.360.B.4.i; A.361.B.4.i; A.362.B.4.i; A.363.B.4.i; A.364.B.4.i;
A.365.B.4.i; A.366.B.4.i; A.367.B.4.i; A.368.B.4.i; A.369.B.4.i; A.370.B.4.i;
A.371.B.4.i; A.372.B.4.i; A.373.B.4.i; A.374.B.4.i; A.375.B.4.i; A.376.B.4.i;
A.377.B.4.i; A.378.B.4.i; A.379.B.4.i; A.380.B.4.i; A.381.B.4.i; A.382.B.4.i;
A.383.B.4.i; A.384.B.4.i; A.385.B.4.i; A.386.B.4.i; A.387.B.4.i; A.388.B.4.i;
A.389.B.4.i; A.390.B.4.i; A.391.B.4.i; A.392.B.4.i; A.393.B.4.i; A.394.B.4.i;
A.395.B.4.i; A.396.B.4.i; A.397.B.4.i; A.398.B.4.i; A.399.B.4.i; A.400.B.4.i;
A.401.B.4.i; A.402.B.4.i; A.403.B.4.i; A.404.B.4.i; A.405.B.4.i; A.406.B.4.i;
A.407.B.4.i; A.408.B.4.i; A.409.B.4.i; A.410.B.4.i; A.411.B.4.i; A.412.B.4.i;
A.413.B.4.i; A.414.B.4.i; A.415.B.4.i; A.416.B.4.i; A.417.B.4.i; A.418.B.4.i;
A.419.B.4.i; A.420.B.4.i; A.421.B.4.i; A.422.B.4.i; A.423.B.4.i; A.424.B.4.i;
A.425.B.4.i; A.426.B.4.i; A.427.B.4.i; A.428.B.4.i; A.429.B.4.i; A.430.B.4.i;
A.431.B.4.i; A.432.B.4.i; A.433.B.4.i; A.434.B.4.i; A.435.B.4.i; A.436.B.4.i;
A.437.B.4.i; A.438.B.4.i; A.439.B.4.i; A.440.B.4.i; A.441.B.4.i; A.442.B.4.i;
A.443.B.4.i; A.444.B.4.i; A.445.B.4.i; A.446.B.4.i; A.447.B.4.i; A.448.B.4.i;
A.449.B.4.i; A.450.B.4.i; A.451.B.4.i; A.452.B.4.i; A.453.B.4.i; A.454.B.4.i;
A.455.B.4.i; A.456.B.4.i; A.457.B.4.i; A.458.B.4.i; A.459.B.4.i; A.460.B.4.i;
A.461.B.4.i; A.462.B.4.i; A.463.B.4.i; A.464.B.4.i; A.465.B.4.i; A.466.B.4.i;
A.467.B.4.i; A.468.B.4.i; A.469.B.4.i; A.470.B.4.i; A.471.B.4.i; A.472.B.4.i;
A.473.B.4.i; A.474.B.4.i; A.475.B.4.i; A.476.B.4.i; A.477.B.4.i; A.478.B.4.i;
A.479.B.4.i; A.480.B.4.i; A.481.B.4.i; A.482.B.4.i; A.483.B.4.i; A.484.B.4.i;
A.485.B.4.i; A.486.B.4.i; A.487.B.4.i; A.488.B.4.i; A.489.B.4.i; A.490.B.4.i;
A.491.B.4.i; A.492.B.4.i; A.493.B.4.i; A.494.B.4.i; A.495.B.4.i; A.496.B.4.i;
A.497.B.4.i; A.498.B.4.i; A.499.B.4.i; A.500.B.4.i; A.501.B.4.i; A.502.B.4.i;
A.503.B.4.i; A.504.B.4.i; A.505.B.4.i; A.506.B.4.i; A.507.B.4.i; A.508.B.4.i;
A.509.B.4.i; A.510.B.4.i; A.511.B.4.i; A.512.B.4.i; A.512.B.4.i; A.513.B.4.i;
A.514.B.4.i; A.515.B.4.i; A.516.B.4.i; A.517.B.4.i; A.518.B.4.i; A.519.B.4.i;
A.520.B.4.i; A.521.B.4.i; A.522.B.4.i; A.523.B.4.i; A.524.B.4.i; A.525.B.4.i;
A.526.B.4.i; A.527.B.4.i; A.528.B.4.i; A.529.B.4.i; A.530.B.4.i; A.531.B.4.i;
A.532.B.4.i; A.533.B.4.i; A.534.B.4.i; A.535.B.4.i; A.536.B.4.i; A.537.B.4.i;
A.538.B.4.i; A.539.B.4.i; A.540.B.4.i; A.541.B.4.i; A.542.B.4.i; A.543.B.4.i;
A.544.B.4.i; A.545.B.4.i; A.546.B.4.i; A.547.B.4.i; A.548.B.4.i; A.549.B.4.i;
A.550.B.4.i; A.551.B.4.i; A.552.B.4.i; A.553.B.4.i; A.554.B.4.i; A.555.B.4.i;
A.556.B.4.i; A.557.B.4.i; A.558.B.4.i; A.559.B.4.i; A.560.B.4.i; A.561.B.4.i;
A.562.B.4.i; A.563.B.4.i; A.564.B.4.i; A.565.B.4.i; A.566.B.4.i; A.567.B.4.i;
A.568.B.4.i; A.569.B.4.i; A.570.B.4.i; A.571.B.4.i; A.572.B.4.i; A.573.B.4.i;
A.574.B.4.i; A.575.B.4.i; A.576.B.4.i; A.577.B.4.i; A.578.B.4.i; A.579.B.4.i;
A.580.B.4.i; A.581.B.4.i; A.582.B.4.i; A.583.B.4.i; A.584.B.4.i; A.585.B.4.i;
A.586.B.4.i; A.587.B.4.i; A.588.B.4.i; A.589.B.4.i; A.590.B.4.i; A.591.B.4.i;
A.592.B.4.i; A.593.B.4.i; A.594.B.4.i; A.595.B.4.i; A.596.B.4.i; A.597.B.4.i;
A.598.B.4.i; A.599.B.4.i; A.600.B.4.i; A.601.B.4.i; A.602.B.4.i; A.603.B.4.i;
A.604.B.4.i; A.605.B.4.i; A.606.B.4.i; A.607.B.4.i; A.608.B.4.i; A.609.B.4.i;
A.610.B.4.i; A.611.B.4.i; A.612.B.4.i; A.613.B.4.i; A.614.B.4.i; A.615.B.4.i;
A.616.B.4.i; A.617.B.4.i; A.618.B.4.i; A.619.B.4.i; A.620.B.4.i; A.621.B.4.i;
A.622.B.4.i; A.623.B.4.i; A.624.B.4.i; A.625.B.4.i; A.626.B.4.i; A.627.B.4.i;
A.628.B.4.i; A.629.B.4.i; A.630.B.4.i; A.631.B.4.i; A.632.B.4.i; A.633.B.4.i;
A.634.B.4.i; A.635.B.4.i; A.636.B.4.i; A.637.B.41; A.638.B.4.i; A.639.B.4.i;
A.640.B.4.i; A.641.B.4.i; A.642.B.4.i; A.643.B.4.i; A.644.B.4.i; A.645.B.4.i;
A.646.B.4.i; A.647.B.4.i; A.648.B.4.i; A.649.B.4.i; A.650.B.4.i; A.651.B.4.i;
A.652.B.4.i; A.653.B.4.i; A.654.B.4.i; A.655.B.4.i; A.656.B.4.i; A.657.B.4.i;
A.658.B.4.i; A.659.B.4.i; A.660.B.4.i; A.2.B.11.i; A.3.B.11.i; A.4.B.11.i; A.5.B.11.i;
A.6.B.11.i; A.7.B.11.i; A.9.B.11.i; A.10.B.11.i; A.15.B.11.i; A.100.B.11.i;
A.101.B.11.i; A.102.B.11.i; A.103.B.11.i; A.104.B.11.i; A.105.B.11.i; A.106.B.11.i;
A.107.B.11.i; A.108.B.11.i; A.109.B.11.i; A.110.B.11.i; A.111.B.11.i; A.112.B.11.i;
A.113.B.11.i; A.114.B.11.i; A.115.B.11.i; A.116.B.11.i; A.117.B.11.i; A.118.B.11.i;
A.119.B.11.i; A.120.B.11.i; A.121.B.11.i; A.122.B.11.i; A.123.B.11.i; A.124.B.11.i;
A.125.B.11.i; A.126.B.11.i; A.127.B.11.i; A.128.B.11.i; A.129.B.11.i; A.130.B.11.i;
A.131.B.11.i; A.132.B.11.i; A.133.B.11.i; A.134.B.11.i; A.135.B.11.i; A.136.B.11.i;
A.137.B.11.i; A.138.B.11.i; A.139.B.11.i; A.140.B.11.i; A.141.B.11.i; A.142.B.11.i;
A.143.B.11.i; A.144.B.11.i; A.145.B.11.i; A.146.B.11.i; A.147.B.11.i; A.148.B.11.i;
A.149.B.11.i; A.150.B.11.i; A.151.B.11.i; A.152.B.11.i; A.153.B.11.i; A.154.B.11.i;
A.155.B.11.i; A.156.B.11.i; A.157.B.11.i; A.158.B.11.i; A.159.B.11.i; A.160.B.11.i;
A.161.B.11.i; A.162.B.11.i; A.163.B.11.i; A.164.B.11.i; A.165.B.11.i; A.166.B.11.i;
A.167.B.11.i; A.168.B.11.i; A.169.B.11.i; A.170.B.11.i; A.171.B.11.i; A.172.B.11.i;
A.173.B.11.i; A.174.B.11.i; A.175.B.11.i; A.176.B.11.i; A.177.B.11.i; A.178.B.11.i;
A.179.B.11.i; A.180.B.11.i; A.181.B.11.i; A.182.B.11.i; A.183.B.11.i; A.184.B.11.i;
A.185.B.11.i; A.186.B.11.i; A.187.B.11.i; A.188.B.11.i; A.189.B.11.i; A.190.B.11.i;
A.191.B.11.i; A.192.B.11.i; A.193.B.11.i; A.194.B.11.i; A.195.B.11.i; A.196.B.11.i;
A.197.B.11.i; A.198.B.11.i; A.199.B.11.i; A.200.B.11.i; A.201.B.11.i; A.202.B.11.i;
A.203.B.11.i; A.204.B.11.i; A.205.B.11.i; A.206.B.11.i; A.207.B.11.i; A.208.B.11.i;
A.209.B.11.i; A.210.B.11.i; A.211.B.11.i; A.212.B.11.i; A.213.B.11.i; A.214.B.11.i;
A.215.B.11.i; A.216.B.11.i; A.217.B.11.i; A.218.B.11.i; A.219.B.11.i; A.220.B.11.i;
A.221.B.11.i; A.222.B.11.i; A.223.B.11.i; A.224.B.11.i; A.225.B.11.i; A.226.B.11.i;
A.227.B.11.i; A.228.B.11.i; A.229.B.11.i; A.230.B.11.i; A.231.B.11.i; A.232.B.11.i;
A.233.B.11.i; A.234.B.11.i; A.235.B.11.i; A.236.B.11.i; A.237.B.11.i; A.238.B.11.i;
A.239.B.11.i; A.240.B.11.i; A.241.B.11.i; A.242.B.11.i; A.243.B.11.i; A.244.B.11.i;
A.245.B.11.i; A.246.B.11.i; A.247.B.11.i; A.248.B.11.i; A.249.B.11.i; A.250.B.11.i;
A.251.B.11.i; A.252.B.11.i; A.253.B.11.i; A.254.B.11.i; A.255.B.11.i; A.256.B.11.i;
A.257.B.11.i; A.258.B.11.i; A.259.B.11.i; A.260.B.11.i; A.261.B.11.i; A.262.B.11.i;
A.263.B.11.i; A.264.B.11.i; A.265.B.11.i; A.266.B.11.i; A.267.B.11.i; A.268.B.11.i;
A.269.B.11.i; A.270.B.11.i; A.271.B.11.i; A.272.B.11.i; A.273.B.11.i; A.274.B.11.i;
A.275.B.11.i; A.276.B.11.i; A.277.B.11.i; A.278.B.11.i; A.279.B.11.i; A.280.B.11.i;
A.281.B.11.i; A.282.B.11.i; A.283.B.11.i; A.284.B.11.i; A.285.B.11.i; A.286.B.11.i;
A.287.B.11.i; A.288.B.11.i; A.289.B.11.i; A.290.B.11.i; A.291.B.11.i; A.292.B.11.i;
A.293.B.11.i; A.294.B.11.i; A.295.B.11.i; A.296.B.11.i; A.297.B.11.i; A.298.B.11.i;
A.299.B.11.i; A.300.B.11.i; A.301.B.11.i; A.302.B.11.i; A.303.B.11.i; A.304.B.11.i;
A.305.B.11.i; A.306.B.11.i; A.307.B.11.i; A.308.B.11.i; A.309.B.11.i; A.310.B.11.i;
A.311.B.11.i; A.312.B.11.i; A.313.B.11.i; A.314.B.11.i; A.315.B.11.i; A.316.B.11.i;
A.317.B.11.i; A.318.B.11.i; A.319.B.11.i; A.320.B.11.i; A.321.B.11.i; A.323.B.11.i;
A.324.B.11.i; A.325.B.11.i; A.326.B.11.i; A.327.B.11.i; A.328.B.11.i; A.329.B.11.i;
A.330.B.11.i; A.331.B.11.i; A.332.B.11.i; A.333.B.11.i; A.334.B.11.i; A.335.B.11.i;
A.336.B.11.i; A.337.B.11.i; A.338.B.11.i; A.339.B.11.i; A.340.B.11.i; A.341.B.11.i;
A.342.B.11.i; A.343.B.11.i; A.344.B.11.i; A.345.B.11.i; A.346.B.11.i; A.347.B.11.i;
A.348.B.11.i; A.349.B.11.i; A.350.B.11.i; A.351.B.11.i; A.352.B.11.i; A.353.B.11.i;
A.354.B.11.i; A.355.B.11.i; A.356.B.11.i; A.357.B.11.i; A.358.B.11.i; A.359.B.11.i;
A.360.B.11.i; A.361.B.11.i; A.362.B.11.i; A.363.B.11.i; A.364.B.11.i; A.365.B.11.i;
A.366.B.11.i; A.367.B.11.i; A.368.B.11.i; A.369.B.11.i; A.370.B.11.i; A.371.B.11.i;
A.372.B.11.i; A.373.B.11.i; A.374.B.11.i; A.375.B.11.i; A.376.B.11.i; A.377.B.11.i;
A.378.B.11.i; A.379.B.11.i; A.380.B.11.i; A.381.B.11.i; A.382.B.11.i; A.383.B.11.i;
A.384.B.11.i; A.385.B.11.i; A.386.B.11.i; A.387.B.11.i; A.388.B.11.i; A.389.B.11.i;
A.390.B.11.i; A.391.B.11.i; A.392.B.11.i; A.393.B.11.i; A.394.B.11.i; A.395.B.11.i;
A.396.B.11.i; A.397.B.11.i; A.398.B.11.i; A.399.B.11.i; A.400.B.11.i; A.401.B.11.i;
A.402.B.11.i; A.403.B.11.i; A.404.B.11.i; A.405.B.11.i; A.406.B.11.i; A.407.B.11.i;
A.408.B.11.i; A.409.B.11.i; A.410.B.11.i; A.411.B.11.i; A.412.B.11.i; A.413.B.11.i;
A.414.B.11.i; A.415.B.11.i; A.416.B.11.i; A.417.B.11.i; A.418.B.11.i; A.419.B.11.i;
A.420.B.11.i; A.421.B.11.i; A.422.B.11.i; A.423.B.11.i; A.424.B.11.i; A.425.B.11.i;
A.426.B.11.i; A.427.B.11.i; A.428.B.11.i; A.429.B.11.i; A.430.B.11.i; A.431.B.11.i;
A.432.B.11.i; A.433.B.11.i; A.434.B.11.i; A.435.B.11.i; A.436.B.11.i; A.437.B.11.i;
A.438.B.11.i; A.439.B.11.i; A.440.B.11.i; A.441.B.11.i; A.442.B.11.i; A.443.B.11.i;
A.444.B.11.i; A.445.B.11.i; A.446.B.11.i; A.447.B.11.i; A.448.B.11.i; A.449.B.11.i;
A.450.B.11.i; A.451.B.11.i; A.452.B.11.i; A.453.B.11.i; A.454.B.11.i; A.455.B.11.i;
A.456.B.11.i; A.457.B.11.i; A.458.B.11.i; A.459.B.11.i; A.460.B.11.i; A.461.B.11.i;
A.462.B.11.i; A.463.B.11.i; A.464.B.11.i; A.465.B.11.i; A.466.B.11.i; A.467.B.11.i;
A.468.B.11.i; A.469.B.11.i; A.470.B.11.i; A.471.B.11.i; A.472.B.11.i; A.473.B.11.i;
A.474.B.11.i; A.475.B.11.i; A.476.B.11.i; A.477.B.11.i; A.478.B.11.i; A.479.B.11.i;
A.480.B.11.i; A.481.B.11.i; A.482.B.11.i; A.483.B.11.i; A.484.B.11.i; A.485.B.11.i;
A.486.B.11.i; A.487.B.11.i; A.488.B.11.i; A.489.B.11.i; A.490.B.11.i; A.491.B.11.i;
A.492.B.11.i; A.493.B.11.i; A.494.B.11.i; A.495.B.11.i; A.496.B.11.i; A.497.B.11.i;
A.498.B.11.i; A.499.B.11.i; A.500.B.11.i; A.501.B.11.i; A.502.B.11.i; A.503.B.11.i;
A.504.B.11.i; A.505.B.11.i; A.506.B.11.i; A.507.B.11.i; A.508.B.11.i; A.509.B.11.i;
A.510.B.11.i; A.511.B.11.i; A.512.B.11.i; A.512.B.11.i; A.513.B.11.i; A.514.B.11.i;
A.515.B.11.i; A.516.B.11.i; A.517.B.11.i; A.518.B.11.i; A.519.B.11.i; A.520.B.11.i;
A.521.B.11.i; A.522.B.11.i; A.523.B.11.i; A.524.B.11.i; A.525.B.11.i; A.526.B.11.i;
A.527.B.11.i; A.528.B.11.i; A.529.B.11.i; A.530.B.11.i; A.531.B.11.i; A.532.B.11.i;
A.533.B.11.i; A.534.B.11.i; A.535.B.11.i; A.536.B.11.i; A.537.B.11.i; A.538.B.11.i;
A.539.B.11.i; A.540.B.11.i; A.541.B.11.i; A.542.B.11.i; A.543.B.11.i; A.544.B.11.i;
A.545.B.11.i; A.546.B.11.i; A.547.B.11.i; A.548.B.11.i; A.549.B.11.i; A.550.B.11.i;
A.551.B.11.i; A.552.B.11.i; A.553.B.11.i; A.554.B.11.i; A.555.B.11.i; A.556.B.11.i;
A.557.B.11.i; A.558.B.11.i; A.559.B.11.i; A.560.B.11.i; A.561.B.11.i; A.562.B.11.i;
A.563.B.11.i; A.564.B.11.i; A.565.B.11.i; A.566.B.11.i; A.567.B.11.i; A.568.B.11.i;
A.569.B.11.i; A.570.B.11.i; A.571.B.11.i; A.572.B.11.i; A.573.B.11.i; A.574.B.11.i;
A.575.B.11.i; A.576.B.11.i; A.577.B.11.i; A.578.B.11.i; A.579.B.11.i; A.580.B.11.i;
A.581.B.11.i; A.582.B.11.i; A.583.B.11.i; A.584.B.11.i; A.585.B.11.i; A.586.B.11.i;
A.587.B.11.i; A.588.B.11.i; A.589.B.11.i; A.590.B.11.i; A.591.B.11.i; A.592.B.11.i;
A.593.B.11.i; A.594.B.11.i; A.595.B.11.i; A.596.B.11.i; A.597.B.11.i; A.598.B.11.i;
A.599.B.11.i; A.600.B.11.i; A.601.B.11.i; A.602.B.11.i; A.603.B.11.i; A.604.B.11.i;
A.605.B.11.i; A.606.B.11.i; A.607.B.11.i; A.608.B.11.i; A.609.B.11.i; A.610.B.11.i;
A.611.B.11.i; A.612.B.11.i; A.613.B.11.i; A.614.B.11.i; A.615.B.11.i; A.616.B.11.i;
A.617.B.11.i; A.618.B.11.i; A.619.B.11.i; A.620.B.11.i; A.621.B.11.i; A.622.B.11.i;
A.623.B.11.i; A.624.B.11.i; A.625.B.11.i; A.626.B.11.i; A.627.B.11.i; A.628.B.11.i;
A.629.B.11.i; A.630.B.11.i; A.631.B.11.i; A.632.B.11.i; A.633.B.11.i; A.634.B.11.i;
A.635.B.11.i; A.636.B.11.i; A.637.B.11.i; A.638.B.11.i; A.639.B.11.i; A.640.B.11.i;
A.641.B.11.i; A.642.B.11.i; A.643.B.11.i; A.644.B.11.i; A.645.B.11.i; A.646.B.11.i;
A.647.B.11.i; A.648.B.11.i; A.649.B.11.i; A.650.B.11.i; A.651.B.11.i; A.652.B.11.i;
A.653.B.11.i; A.654.B.11.i; A.655.B.11.i; A.656.B.11.i; A.657.B.11.i; A.658.B.11.i;
A.659.B.11.i; A.660.B.11.i; A.2.C.4.i; A.3.C.4.i; A.4.C.4.i; A.5.C.4.i; A.6.C.4.i;
A.7.C.4.i; A.9.C.4.i; A.10.C.4.i; A.15.C.4.i; A.100.C.4.i; A.101.C.4.i; A.102.C.4.i;
A.103.C.4.i; A.104.C.4.i; A.105.C.4.i; A.106.C.4.i; A.107.C.4.i; A.108.C.4.i;
A.109.C.4.i; A.110.C.4.i; A.111.C.4.i; A.112.C.4.i; A.113.C.4.i; A.114.C.4.i;
A.115.C.4.i; A.116.C.4.i; A.117.C.4.i; A.118.C.4.i; A.119.C.4.i; A.120.C.4.i;
A.121.C.4.i; A.122.C.4.i; A.123.C.4.i; A.124.C.4.i; A.125.C.4.i; A.126.C.4.i;
A.127.C.4.i; A.128.C.4.i; A.129.C.4.i; A.130.C.4.i; A.131.C.4.i; A.132.C.4.i;
A.133.C.4.i; A.134.C.4.i; A.135.C.4.i; A.136.C.4.i; A.137.C.4.i; A.138.C.4.i;
A.139.C.4.i; A.140.C.4.i; A.141.C.4.i; A.142.C.4.i; A.143.C.4.i; A.144.C.4.i;
A.145.C.4.i; A.146.C.4.i; A.147.C.4.i; A.148.C.4.i; A.149.C.4.i; A.150.C.4.i;
A.151.C.4.i; A.152.C.4.i; A.153.C.4.i; A.154.C.4.i; A.155.C.4.i; A.156.C.4.i;
A.157.C.4.i; A.158.C.4.i; A.159.C.4.i; A.160.C.4.i; A.161.C.4.i; A.162.C.4.i;
A.163.C.4.i; A.164.C.4.i; A.165.C.4.i; A.166.C.4.i; A.167.C.4.i; A.168.C.4.i;
A.169.C.4.i; A.170.C.4.i; A.171.C.4.i; A.172.C.4.i; A.173.C.4.i; A.174.C.4.i;
A.175.C.4.i; A.176.C.4.i; A.177.C.4.i; A.178.C.4.i; A.179.C.4.i; A.180.C.4.i;
A.181.C.4.i; A.182.C.4.i; A.183.C.4.i; A.184.C.4.i; A.185.C.4.i; A.186.C.4.i;
A.187.C.4.i; A.188.C.4.i; A.189.C.4.i; A.190.C.4.i; A.191.C.4.i; A.192.C.4.i;
A.193.C.4.i; A.194.C.4.i; A.195.C.4.i; A.196.C.4.i; A.197.C.4.i; A.198.C.4.i;
A.199.C.4.i; A.200.C.4.i; A.201.C.4.i; A.202.C.4.i; A.203.C.4.i; A.204.C.4.i;
A.205.C.4.i; A.206.C.4.i; A.207.C.4.i; A.208.C.4.i; A.209.C.4.i; A.210.C.4.i;
A.211.C.4.i; A.212.C.4.i; A.213.C.4.i; A.214.C.4.i; A.215.C.4.i; A.216.C.4.i;
A.217.C.4.i; A.218.C.4.i; A.219.C.4.i; A.220.C.4.i; A.221.C.4.i; A.222.C.4.i;
A.223.C.4.i; A.224.C.4.i; A.225.C.4.i; A.226.C.4.i; A.227.C.4.i; A.228.C.4.i;
A.229.C.4.i; A.230.C.4.i; A.231.C.4.i; A.232.C.4.i; A.233.C.4.i; A.234.C.4.i;
A.235.C.4.i; A.236.C.4.i; A.237.C.4.i; A.238.C.4.i; A.239.C.4.i; A.240.C.4.i;
A.241.C.4.i; A.242.C.4.i; A.243.C.4.i; A.244.C.4.i; A.245.C.4.i; A.246.C.4.i;
A.247.C.4.i; A.248.C.4.i; A.249.C.4.i; A.250.C.4.i; A.251.C.4.i; A.252.C.4.i;
A.253.C.4.i; A.254.C.4.i; A.255.C.4.i; A.256.C.4.i; A.257.C.4.i; A.258.C.4.i;
A.259.C.4.i; A.260.C.4.i; A.261.C.4.i; A.262.C.4.i; A.263.C.4.i; A.264.C.4.i;
A.265.C.4.i; A.266.C.4.i; A.267.C.4.i; A.268.C.4.i; A.269.C.4.i; A.270.C.4.i;
A.271.C.4.i; A.272.C.4.i; A.273.C.4.i; A.274.C.4.i; A.275.C.4.i; A.276.C.4.i;
A.277.C.4.i; A.278.C.4.i; A.279.C.4.i; A.280.C.4.i; A.281.C.4.i; A.282.C.4.i;
A.283.C.4.i; A.284.C.4.i; A.285.C.4.i; A.286.C.4.i; A.287.C.4.i; A.288.C.4.i;
A.289.C.4.i; A.290.C.4.i; A.291.C.4.i; A.292.C.4.i; A.293.C.4.i; A.294.C.4.i;
A.295.C.4.i; A.296.C.4.i; A.297.C.4.i; A.298.C.4.i; A.299.C.4.i; A.300.C.4.i;
A.301.C.4.i; A.302.C.4.i; A.303.C.4.i; A.304.C.4.i; A.305.C.4.i; A.306.C.4.i;
A.307.C.4.i; A.308.C.4.i; A.309.C.4.i; A.310.C.4.i; A.311.C.4.i; A.312.C.4.i;
A.313.C.4.i; A.314.C.4.i; A.315.C.4.i; A.316.C.4.i; A.317.C.4.i; A.318.C.4.i;
A.319.C.4.i; A.320.C.4.i; A.321.C.4.i; A.323.C.4.i; A.324.C.4.i; A.325.C.4.i;
A.326.C.4.i; A.327.C.4.i; A.328.C.4.i; A.329.C.4.i; A.330.C.4.i; A.331.C.4.i;
A.332.C.4.i; A.333.C.4.i; A.334.C.4.i; A.335.C.4.i; A.336.C.4.i; A.337.C.4.i;
A.338.C.4.i; A.339.C.4.i; A.340.C.4.i; A.341.C.4.i; A.342.C.4.i; A.343.C.4.i;
A.344.C.4.i; A.345.C.4.i; A.346.C.4.i; A.347.C.4.i; A.348.C.4.i; A.349.C.4.i;
A.350.C.4.i; A.351.C.4.i; A.352.C.4.i; A.353.C.4.i; A.354.C.4.i; A.355.C.4.i;
A.356.C.4.i; A.357.C.4.i; A.358.C.4.i; A.359.C.4.i; A.360.C.4.i; A.361.C.4.i;
A.362.C.4.i; A.363.C.4.i; A.364.C.4.i; A.365.C.4.i; A.366.C.4.i; A.367.C.4.i;
A.368.C.4.i; A.369.C.4.i; A.370.C.4.i; A.371.C.4.i; A.372.C.4.i; A.373.C.4.i;
A.374.C.4.i; A.375.C.4.i; A.376.C.4.i; A.377.C.4.i; A.378.C.4.i; A.379.C.4.i;
A.380.C.4.i; A.381.C.4.i; A.382.C.4.i; A.383.C.4.i; A.384.C.4.i; A.385.C.4.i;
A.386.C.4.i; A.387.C.4.i; A.388.C.4.i; A.389.C.4.i; A.390.C.4.i; A.391.C.4.i;
A.392.C.4.i; A.393.C.4.i; A.394.C.4.i; A.395.C.4.i; A.396.C.4.i; A.397.C.4.i;
A.398.C.4.i; A.399.C.4.i; A.400.C.4.i; A.401.C.4.i; A.402.C.4.i; A.403.C.4.i;
A.404.C.4.i; A.405.C.4.i; A.406.C.4.i; A.407.C.4.i; A.408.C.4.i; A.409.C.4.i;
A.410.C.4.i; A.411.C.4.i; A.412.C.4.i; A.413.C.4.i; A.414.C.4.i; A.415.C.4.i;
A.416.C.4.i; A.417.C.4.i; A.418.C.4.i; A.419.C.4.i; A.420.C.4.i; A.421.C.4.i;
A.422.C.4.i; A.423.C.4.i; A.424.C.4.i; A.425.C.4.i; A.426.C.4.i; A.427.C.4.i;
A.428.C.4.i; A.429.C.4.i; A.430.C.4.i; A.431.C.4.i; A.432.C.4.i; A.433.C.4.i;
A.434.C.4.i; A.435.C.4.i; A.436.C.4.i; A.437.C.4.i; A.438.C.4.i; A.439.C.4.i;
A.440.C.4.i; A.441.C.4.i; A.442.C.4.i; A.443.C.4.i; A.444.C.4.i; A.445.C.4.i;
A.446.C.4.i; A.447.C.4.i; A.448.C.4.i; A.449.C.4.i; A.450.C.4.i; A.451.C.4.i;
A.452.C.4.i; A.453.C.4.i; A.454.C.4.i; A.455.C.4.i; A.456.C.4.i; A.457.C.4.i;
A.458.C.4.i; A.459.C.4.i; A.460.C.4.i; A.461.C.4.i; A.462.C.4.i; A.463.C.4.i;
A.464.C.4.i; A.465.C.4.i; A.466.C.4.i; A.467.C.4.i; A.468.C.4.i; A.469.C.4.i;
A.470.C.4.i; A.471.C.4.i; A.472.C.4.i; A.473.C.4.i; A.474.C.4.i; A.475.C.4.i;
A.476.C.4.i; A.477.C.4.i; A.478.C.4.i; A.479.C.4.i; A.480.C.4.i; A.481.C.4.i;
A.482.C.4.i; A.483.C.4.i; A.484.C.4.i; A.485.C.4.i; A.486.C.4.i; A.487.C.4.i;
A.488.C.4.i; A.489.C.4.i; A.490.C.4.i; A.491.C.4.i; A.492.C.4.i; A.493.C.4.i;
A.494.C.4.i; A.495.C.4.i; A.496.C.4.i; A.497.C.4.i; A.498.C.4.i; A.499.C.4.i;
A.500.C.4.i; A.501.C.4.i; A.502.C.4.i; A.503.C.4.i; A.504.C.4.i; A.505.C.4.i;
A.506.C.4.i; A.507.C.4.i; A.508.C.4.i; A.509.C.4.i; A.510.C.4.i; A.511.C.4.i;
A.512.C.4.i; A.512.C.4.i; A.513.C.4.i; A.514.C.4.i; A.515.C.4.i; A.516.C.4.i;
A.517.C.4.i; A.518.C.4.i; A.519.C.4.i; A.520.C.4.i; A.521.C.4.i; A.522.C.4.i;
A.523.C.4.i; A.524.C.4.i; A.525.C.4.i; A.526.C.4.i; A.527.C.4.i; A.528.C.4.i;
A.529.C.4.i; A.530.C.4.i; A.531.C.4.i; A.532.C.4.i; A.533.C.4.i; A.534.C.4.i;
A.535.C.4.i; A.536.C.4.i; A.537.C.4.i; A.538.C.4.i; A.539.C.4.i; A.540.C.4.i;
A.541.C.4.i; A.542.C.4.i; A.543.C.4.i; A.544.C.4.i; A.545.C.4.i; A.546.C.4.i;
A.547.C.4.i; A.548.C.4.i; A.549.C.4.i; A.550.C.4.i; A.551.C.4.i; A.552.C.4.i;
A.553.C.4.i; A.554.C.4.i; A.555.C.4.i; A.556.C.4.i; A.557.C.4.i; A.558.C.4.i;
A.559.C.4.i; A.560.C.4.i; A.561.C.4.i; A.562.C.4.i; A.563.C.4.i; A.564.C.4.i;
A.565.C.4.i; A.566.C.4.i; A.567.C.4.i; A.568.C.4.i; A.569.C.4.i; A.570.C.4.i;
A.571.C.4.i; A.572.C.4.i; A.573.C.4.i; A.574.C.4.i; A.575.C.4.i; A.576.C.4.i;
A.577.C.4.i; A.578.C.4.i; A.579.C.4.i; A.580.C.4.i; A.581.C.4.i; A.582.C.4.i;
A.583.C.4.i; A.584.C.4.i; A.585.C.4.i; A.586.C.4.i; A.587.C.4.i; A.588.C.4.i;
A.589.C.4.i; A.590.C.4.i; A.591.C.4.i; A.592.C.4.i; A.593.C.4.i; A.594.C.4.i;
A.595.C.4.i; A.596.C.4.i; A.597.C.4.i; A.598.C.4.i; A.599.C.4.i; A.600.C.4.i;
A.601.C.4.i; A.602.C.4.i; A.603.C.4.i; A.604.C.4.i; A.605.C.4.i; A.606.C.4.i;
A.607.C.4.i; A.608.C.4.i; A.609.C.4.i; A.610.C.4.i; A.611.C.4.i; A.612.C.4.i;
A.613.C.4.i; A.614.C.4.i; A.615.C.4.i; A.616.C.4.i; A.617.C.4.i; A.618.C.4.i;
A.619.C.4.i; A.620.C.4.i; A.621.C.4.i; A.622.C.4.i; A.623.C.4.i; A.624.C.4.i;
A.625.C.4.i; A.626.C.4.i; A.627.C.4.i; A.628.C.4.i; A.629.C.4.i; A.630.C.4.i;
A.631.C.4.i; A.632.C.4.i; A.633.C.4.i; A.634.C.4.i; A.635.C.4.i; A.636.C.4.i;
A.637.C.4.i; A.638.C.4.i; A.639.C.4.i; A.640.C.4.i; A.641.C.4.i; A.642.C.4.i;
A.643.C.4.i; A.644.C.4.i; A.645.C.4.i; A.646.C.4.i; A.647.C.4.i; A.648.C.4.i;
A.649.C.4.i; A.650.C.4.i; A.651.C.4.i; A.652.C.4.i; A.653.C.4.i; A.654.C.4.i;
A.655.C.4.i; A.656.C.4.i; A.657.C.4.i; A.658.C.4.i; A.659.C.4.i; A.660.C.4.i;
A.2.C.11.i; A.3.C.11.i; A.4.C.11.i; A.5.C.11.i; A.6.C.11.i; A.7.C.11.i; A.9.C.11.i;
A.10.C.11.i; A.15.C.11.i; A.100.C.11.i; A.101.C.11.i; A.102.C.11.i; A.103.C.11.i;
A.104.C.11.i; A.105.C.11.i; A.106.C.11.i; A.107.C.11.i; A.108.C.11.i; A.109.C.11.i;
A.110.C.11.i; A.111.C.11.i; A.112.C.11.i; A.113.C.11.i; A.114.C.11.i; A.115.C.11.i;
A.116.C.11.i; A.117.C.11.i; A.118.C.11.i; A.119.C.11.i; A.120.C.11.i; A.121.C.11.i;
A.122.C.11.i; A.123.C.11.i; A.124.C.11.i; A.125.C.11.i; A.126.C.11.i; A.127.C.11.i;
A.128.C.11.i; A.129.C.11.i; A.130.C.11.i; A.131.C.11.i; A.132.C.11.i; A.133.C.11.i;
A.134.C.11.i; A.135.C.11.i; A.136.C.11.i; A.137.C.11.i; A.138.C.11.i; A.139.C.11.i;
A.140.C.11.i; A.141.C.11.i; A.142.C.11.i; A.143.C.11.i; A.144.C.11.i; A.145.C.11.i;
A.146.C.11.i; A.147.C.11.i; A.148.C.11.i; A.149.C.11.i; A.150.C.11.i; A.151.C.11.i;
A.152.C.11.i; A.153.C.11.i; A.154.C.11.i; A.155.C.11.i; A.156.C.11.i; A.157.C.11.i;
A.158.C.11.i; A.159.C.11.i; A.160.C.11.i; A.161.C.11.i; A.162.C.11.i; A.163.C.11.i;
A.164.C.11.i; A.165.C.11.i; A.166.C.11.i; A.167.C.11.i; A.168.C.11.i; A.169.C.11.i;
A.170.C.11.i; A.171.C.11.i; A.172.C.11.i; A.173.C.11.i; A.174.C.11.i; A.175.C.11.i;
A.176.C.11.i; A.177.C.11.i; A.178.C.11.i; A.179.C.11.i; A.180.C.11.i; A.181.C.11.i;
A.182.C.11.i; A.183.C.11.i; A.184.C.11.i; A.185.C.11.i; A.186.C.11.i; A.187.C.11.i;
A.188.C.11.i; A.189.C.11.i; A.190.C.11.i; A.191.C.11.i; A.192.C.11.i; A.193.C.11.i;
A.194.C.11.i; A.195.C.11.i; A.196.C.11.i; A.197.C.11.i; A.198.C.11.i; A.199.C.11.i;
A.200.C.11.i; A.201.C.11.i; A.202.C.11.i; A.203.C.11.i; A.204.C.11.i; A.205.C.11.i;
A.206.C.11.i; A.207.C.11.i; A.208.C.11.i; A.209.C.11.i; A.210.C.11.i; A.211.C.11.i;
A.212.C.11.i; A.213.C.11.i; A.214.C.11.i; A.215.C.11.i; A.216.C.11.i; A.217.C.11.i;
A.218.C.11.i; A.219.C.11.i; A.220.C.11.i; A.221.C.11.i; A.222.C.11.i; A.223.C.11.i;
A.224.C.11.i; A.225.C.11.i; A.226.C.11.i; A.227.C.11.i; A.228.C.11.i; A.229.C.11.i;
A.230.C.11.i; A.231.C.11.i; A.232.C.11.i; A.233.C.11.i; A.234.C.11.i; A.235.C.11.i;
A.236.C.11.i; A.237.C.11.i; A.238.C.11.i; A.239.C.11.i; A.240.C.11.i; A.241.C.11.i;
A.242.C.11.i; A.243.C.11.i; A.244.C.11.i; A.245.C.11.i; A.246.C.11.i; A.247.C.11.i;
A.248.C.11.i; A.249.C.11.i; A.250.C.11.i; A.251.C.11.i; A.252.C.11.i; A.253.C.11.i;
A.254.C.11.i; A.255.C.11.i; A.256.C.11.i; A.257.C.11.i; A.258.C.11.i; A.259.C.11.i;
A.260.C.11.i; A.261.C.11.i; A.262.C.11.i; A.263.C.11.i; A.264.C.11.i; A.265.C.11.i;
A.266.C.11.i; A.267.C.11.i; A.268.C.11.i; A.269.C.11.i; A.270.C.11.i; A.271.C.11.i;
A.272.C.11.i; A.273.C.11.i; A.274.C.11.i; A.275.C.11.i; A.276.C.11.i; A.277.C.11.i;
A.278.C.11.i; A.279.C.11.i; A.280.C.11.i; A.281.C.11.i; A.282.C.11.i; A.283.C.11.i;
A.284.C.11.i; A.285.C.11.i; A.286.C.11.i; A.287.C.11.i; A.288.C.11.i; A.289.C.11.i;
A.290.C.11.i; A.291.C.11.i; A.292.C.11.i; A.293.C.11.i; A.294.C.11.i; A.295.C.11.i;
A.296.C.11.i; A.297.C.11.i; A.298.C.11.i; A.299.C.11.i; A.300.C.11.i; A.301.C.11.i;
A.302.C.11.i; A.303.C.11.i; A.304.C.11.i; A.305.C.11.i; A.306.C.11.i; A.307.C.11.i;
A.308.C.11.i; A.309.C.11.i; A.310.C.11.i; A.311.C.11.i; A.312.C.11.i; A.313.C.11.i;
A.314.C.11.i; A.315.C.11.i; A.316.C.11.i; A.317.C.11.i; A.318.C.11.i; A.319.C.11.i;
A.320.C.11.i; A.321.C.11.i; A.323.C.11.i; A.324.C.11.i; A.325.C.11.i; A.326.C.11.i;
A.327.C.11.i; A.328.C.11.i; A.329.C.11.i; A.330.C.11.i; A.331.C.11.i; A.332.C.11.i;
A.333.C.11.i; A.334.C.11.i; A.335.C.11.i; A.336.C.11.i; A.337.C.11.i; A.338.C.11.i;
A.339.C.11.i; A.340.C.11.i; A.341.C.11.i; A.342.C.11.i; A.343.C.11.i; A.344.C.11.i;
A.345.C.11.i; A.346.C.11.i; A.347.C.11.i; A.348.C.11.i; A.349.C.11.i; A.350.C.11.i;
A.351.C.11.i; A.352.C.11.i; A.353.C.11.i; A.354.C.11.i; A.355.C.11.i; A.356.C.11.i;
A.357.C.11.i; A.358.C.11.i; A.359.C.11.i; A.360.C.11.i; A.361.C.11.i; A.362.C.11.i;
A.363.C.11.i; A.364.C.11.i; A.365.C.11.i; A.366.C.11.i; A.367.C.11.i; A.368.C.11.i;
A.369.C.11.i; A.370.C.11.i; A.371.C.11.i; A.372.C.11.i; A.373.C.11.i; A.374.C.11.i;
A.375.C.11.i; A.376.C.11.i; A.377.C.11.i; A.378.C.11.i; A.379.C.11.i; A.380.C.11.i;
A.381.C.11.i; A.382.C.11.i; A.383.C.11.i; A.384.C.11.i; A.385.C.11.i; A.386.C.11.i;
A.387.C.11.i; A.388.C.11.i; A.389.C.11.i; A.390.C.11.i; A.391.C.11.i; A.392.C.11.i;
A.393.C.11.i; A.394.C.11.i; A.395.C.11.i; A.396.C.11.i; A.397.C.11.i; A.398.C.11.i;
A.399.C.11.i; A.400.C.11.i; A.401.C.11.i; A.402.C.11.i; A.403.C.11.i; A.404.C.11.i;
A.405.C.11.i; A.406.C.11.i; A.407.C.11.i; A.408.C.11.i; A.409.C.11.i; A.410.C.11.i;
A.411.C.11.i; A.412.C.11.i; A.413.C.11.i; A.414.C.11.i; A.415.C.11.i; A.416.C.11.i;
A.417.C.11.i; A.418.C.11.i; A.419.C.11.i; A.420.C.11.i; A.421.C.11.i; A.422.C.11.i;
A.423.C.11.i; A.424.C.11.i; A.425.C.11.i; A.426.C.11.i; A.427.C.11.i; A.428.C.11.i;
A.429.C.11.i; A.430.C.11.i; A.431.C.11.i; A.432.C.11.i; A.433.C.11.i; A.434.C.11.i;
A.435.C.11.i; A.436.C.11.i; A.437.C.11.i; A.438.C.11.i; A.439.C.11.i; A.440.C.11.i;
A.441.C.11.i; A.442.C.11.i; A.443.C.11.i; A.444.C.11.i; A.445.C.11.i; A.446.C.11.i;
A.447.C.11.i; A.448.C.11.i; A.449.C.11.i; A.450.C.11.i; A.451.C.11.i; A.452.C.11.i;
A.453.C.11.i; A.454.C.11.i; A.455.C.11.i; A.456.C.11.i; A.457.C.11.i; A.458.C.11.i;
A.459.C.11.i; A.460.C.11.i; A.461.C.11.i; A.462.C.11.i; A.463.C.11.i; A.464.C.11.i;
A.465.C.11.i; A.466.C.11.i; A.467.C.11.i; A.468.C.11.i; A.469.C.11.i; A.470.C.11.i;
A.471.C.11.i; A.472.C.11.i; A.473.C.11.i; A.474.C.11.i; A.475.C.11.i; A.476.C.11.i;
A.477.C.11.i; A.478.C.11.i; A.479.C.11.i; A.480.C.11.i; A.481.C.11.i; A.482.C.11.i;
A.483.C.11.i; A.484.C.11.i; A.485.C.11.i; A.486.C.11.i; A.487.C.11.i; A.488.C.11.i;
A.489.C.11.i; A.490.C.11.i; A.491.C.11.i; A.492.C.11.i; A.493.C.11.i; A.494.C.11.i;
A.495.C.11.i; A.496.C.11.i; A.497.C.11.i; A.498.C.11.i; A.499.C.11.i; A.500.C.11.i;
A.501.C.11.i; A.502.C.11.i; A.503.C.11.i; A.504.C.11.i; A.505.C.11.i; A.506.C.11.i;
A.507.C.11.i; A.508.C.11.i; A.509.C.11.i; A.510.C.11.i; A.511.C.11.i; A.512.C.11.i;
A.512.C.11.i; A.513.C.11.i; A.514.C.11.i; A.515.C.11.i; A.516.C.11.i; A.517.C.11.i;
A.518.C.11.i; A.519.C.11.i; A.520.C.11.i; A.521.C.11.i; A.522.C.11.i; A.523.C.11.i;
A.524.C.11.i; A.525.C.11.i; A.526.C.11.i; A.527.C.11.i; A.528.C.11.i; A.529.C.11.i;
A.530.C.11.i; A.531.C.11.i; A.532.C.11.i; A.533.C.11.i; A.534.C.11.i; A.535.C.11.i;
A.536.C.11.i; A.537.C.11.i; A.538.C.11.i; A.539.C.11.i; A.540.C.11.i; A.541.C.11.i;
A.542.C.11.i; A.543.C.11.i; A.544.C.11.i; A.545.C.11.i; A.546.C.11.i; A.547.C.11.i;
A.548.C.11.i; A.549.C.11.i; A.550.C.11.i; A.551.C.11.i; A.552.C.11.i; A.553.C.11.i;
A.554.C.11.i; A.555.C.11.i; A.556.C.11.i; A.557.C.11.i; A.558.C.11.i; A.559.C.11.i;
A.560.C.11.i; A.561.C.11.i; A.562.C.11.i; A.563.C.11.i; A.564.C.11.i; A.565.C.11.i;
A.566.C.11.i; A.567.C.11.i; A.568.C.11.i; A.569.C.11.i; A.570.C.11.i; A.571.C.11.i;
A.572.C.11.i; A.573.C.11.i; A.574.C.11.i; A.575.C.11.i; A.576.C.11.i; A.577.C.11.i;
A.578.C.11.i; A.579.C.11.i; A.580.C.11.i; A.581.C.11.i; A.582.C.11.i; A.583.C.11.i;
A.584.C.11.i; A.585.C.11.i; A.586.C.11.i; A.587.C.11.i; A.588.C.11.i; A.589.C.11.i;
A.590.C.11.i; A.591.C.11.i; A.592.C.11.i; A.593.C.11.i; A.594.C.11.i; A.595.C.11.i;
A.596.C.11.i; A.597.C.11.i; A.598.C.11.i; A.599.C.11.i; A.600.C.11.i; A.601.C.11.i;
A.602.C.11.i; A.603.C.11.i; A.604.C.11.i; A.605.C.11.i; A.606.C.11.i; A.607.C.11.i;
A.608.C.11.i; A.609.C.11.i; A.610.C.11.i; A.611.C.11.i; A.612.C.11.i; A.613.C.11.i;
A.614.C.11.i; A.615.C.11.i; A.616.C.11.i; A.617.C.11.i; A.618.C.11.i; A.619.C.11.i;
A.620.C.11.i; A.621.C.11.i; A.622.C.11.i; A.623.C.11.i; A.624.C.11.i; A.625.C.11.i;
A.626.C.11.i; A.627.C.11.i; A.628.C.11.i; A.629.C.11.i; A.630.C.11.i; A.631.C.11.i;
A.632.C.11.i; A.633.C.11.i; A.634.C.11.i; A.635.C.11.i; A.636.C.11.i; A.637.C.11.i;
A.638.C.11.i; A.639.C.11.i; A.640.C.11.i; A.641.C.11.i; A.642.C.11.i; A.643.C.11.i;
A.644.C.11.i; A.645.C.11.i; A.646.C.11.i; A.647.C.11.i; A.648.C.11.i; A.649.C.11.i;
A.650.C.11.i; A.651.C.11.i; A.652.C.11.i; A.653.C.11.i; A.654.C.11.i; A.655.C.11.i;
A.656.C.11.i; A.657.C.11.i; A.658.C.11.i; A.659.C.11.i; A.660.C.11.i; A.2.D.4.i;
A.3.D.4.i; A.4.D.4.i; A.5.D.4.i; A.6.D.4.i; A.7.D.4.i; A.9.D.4.i; A.10.D.4.i;
A.15.D.4.i; A.100.D.4.i; A.101.D.4.i; A.102.D.4.i; A.103.D.4.i; A.104.D.4.i;
A.105.D.4.i; A.106.D.4.i; A.107.D.4.i; A.108.D.4.i; A.109.D.4.i; A.110.D.4.i;
A.111.D.4.i; A.112.D.4.i; A.113.D.4.i; A.114.D.4.i; A.115.D.4.i; A.116.D.4.i;
A.117.D.4.i; A.118.D.4.i; A.119.D.4.i; A.120.D.4.i; A.121.D.4.i; A.122.D.4.i;
A.123.D.4.i; A.124.D.4.i; A.125.D.4.i; A.126.D.4.i; A.127.D.4.i; A.128.D.4.i;
A.129.D.4.i; A.130.D.4.i; A.131.D.4.i; A.132.D.4.i; A.133.D.4.i; A.134.D.4.i;
A.135.D.4.i; A.136.D.4.i; A.137.D.4.i; A.138.D.4.i; A.139.D.4.i; A.140.D.4.i;
A.141.D.4.i; A.142.D.4.i; A.143.D.4.i; A.144.D.4.i; A.145.D.4.i; A.146.D.4.i;
A.147.D.4.i; A.148.D.4.i; A.149.D.4.i; A.150.D.4.i; A.151.D.4.i; A.152.D.4.i;
A.153.D.4.i; A.154.D.4.i; A.155.D.4.i; A.156.D.4.i; A.157.D.4.i; A.158.D.4.i;
A.159.D.4.i; A.160.D.4.i; A.161.D.4.i; A.162.D.4.i; A.163.D.4.i; A.164.D.4.i;
A.165.D.4.i; A.166.D.4.i; A.167.D.4.i; A.168.D.4.i; A.169.D.4.i; A.170.D.4.i;
A.171.D.4.i; A.172.D.4.i; A.173.D.4.i; A.174.D.4.i; A.175.D.4.i; A.176.D.4.i;
A.177.D.4.i; A.178.D.4.i; A.179.D.4.i; A.180.D.4.i; A.181.D.4.i; A.182.D.4.i;
A.183.D.4.i; A.184.D.4.i; A.185.D.4.i; A.186.D.4.i; A.187.D.4.i; A.188.D.4.i;
A.189.D.4.i; A.190.D.4.i; A.191.D.4.i; A.192.D.4.i; A.193.D.4.i; A.194.D.4.i;
A.195.D.4.i; A.196.D.4.i; A.197.D.4.i; A.198.D.4.i; A.199.D.4.i; A.200.D.4.i;
A.201.D.4.i; A.202.D.4.i; A.203.D.4.i; A.204.D.4.i; A.205.D.4.i; A.206.D.4.i;
A.207.D.4.i; A.208.D.4.i; A.209.D.4.i; A.210.D.4.i; A.211.D.4.i; A.212.D.4.i;
A.213.D.4.i; A.214.D.4.i; A.215.D.4.i; A.216.D.4.i; A.217.D.4.i; A.218.D.4.i;
A.219.D.4.i; A.220.D.4.i; A.221.D.4.i; A.222.D.4.i; A.223.D.4.i; A.224.D.4.i;
A.225.D.4.i; A.226.D.4.i; A.227.D.4.i; A.228.D.4.i; A.229.D.4.i; A.230.D.4.i;
A.231.D.4.i; A.232.D.4.i; A.233.D.4.i; A.234.D.4.i; A.235.D.4.i; A.236.D.4.i;
A.237.D.4.i; A.238.D.4.i; A.239.D.4.i; A.240.D.4.i; A.241.D.4.i; A.242.D.4.i;
A.243.D.4.i; A.244.D.4.i; A.245.D.4.i; A.246.D.4.i; A.247.D.4.i; A.248.D.4.i;
A.249.D.4.i; A.250.D.4.i; A.251.D.4.i; A.252.D.4.i; A.253.D.4.i; A.254.D.4.i;
A.255.D.4.i; A.256.D.4.i; A.257.D.4.i; A.258.D.4.i; A.259.D.4.i; A.260.D.4.i;
A.261.D.4.i; A.262.D.4.i; A.263.D.4.i; A.264.D.4.i; A.265.D.4.i; A.266.D.4.i;
A.267.D.4.i; A.268.D.4.i; A.269.D.4.i; A.270.D.4.i; A.271.D.4.i; A.272.D.4.i;
A.273.D.4.i; A.274.D.4.i; A.275.D.4.i; A.276.D.4.i; A.277.D.4.i; A.278.D.4.i;
A.279.D.4.i; A.280.D.4.i; A.281.D.4.i; A.282.D.4.i; A.283.D.4.i; A.284.D.4.i;
A.285.D.4.i; A.286.D.4.i; A.287.D.4.i; A.288.D.4.i; A.289.D.4.i; A.290.D.4.i;
A.291.D.4.i; A.292.D.4.i; A.293.D.4.i; A.294.D.4.i; A.295.D.4.i; A.296.D.4.i;
A.297.D.4.i; A.298.D.4.i; A.299.D.4.i; A.300.D.4.i; A.301.D.4.i; A.302.D.4.i;
A.303.D.4.i; A.304.D.4.i; A.305.D.4.i; A.306.D.4.i; A.307.D.4.i; A.308.D.4.i;
A.309.D.4.i; A.310.D.4.i; A.311.D.4.i; A.312.D.4.i; A.313.D.4.i; A.314.D.4.i;
A.315.D.4.i; A.316.D.4.i; A.317.D.4.i; A.318.D.4.i; A.319.D.4.i; A.320.D.4.i;
A.321.D.4.i; A.323.D.4.i; A.324.D.4.i; A.325.D.4.i; A.326.D.4.i; A.327.D.4.i;
A.328.D.4.i; A.329.D.4.i; A.330.D.4.i; A.331.D.4.i; A.332.D.4.i; A.333.D.4.i;
A.334.D.4.i; A.335.D.4.i; A.336.D.4.i; A.337.D.4.i; A.338.D.4.i; A.339.D.4.i;
A.340.D.4.i; A.341.D.4.i; A.342.D.4.i; A.343.D.4.i; A.344.D.4.i; A.345.D.4.i;
A.346.D.4.i; A.347.D.4.i; A.348.D.4.i; A.349.D.4.i; A.350.D.4.i; A.351.D.4.i;
A.352.D.4.i; A.353.D.4.i; A.354.D.4.i; A.355.D.4.i; A.356.D.4.i; A.357.D.4.i;
A.358.D.4.i; A.359.D.4.i; A.360.D.4.i; A.361.D.4.i; A.362.D.4.i; A.363.D.4.i;
A.364.D.4.i; A.365.D.4.i; A.366.D.4.i; A.367.D.4.i; A.368.D.4.i; A.369.D.4.i;
A.370.D.4.i; A.371.D.4.i; A.372.D.4.i; A.373.D.4.i; A.374.D.4.i; A.375.D.4.i;
A.376.D.4.i; A.377.D.4.i; A.378.D.4.i; A.379.D.4.i; A.380.D.4.i; A.381.D.4.i;
A.382.D.4.i; A.383.D.4.i; A.384.D.4.i; A.385.D.4.i; A.386.D.4.i; A.387.D.4.i;
A.388.D.4.i; A.389.D.4.i; A.390.D.4.i; A.391.D.4.i; A.392.D.4.i; A.393.D.4.i;
A.394.D.4.i; A.395.D.4.i; A.396.D.4.i; A.397.D.4.i; A.398.D.4.i; A.399.D.4.i;
A.400.D.4.i; A.401.D.4.i; A.402.D.4.i; A.403.D.4.i; A.404.D.4.i; A.405.D.4.i;
A.406.D.4.i; A.407.D.4.i; A.408.D.4.i; A.409.D.4.i; A.410.D.4.i; A.411.D.4.i;
A.412.D.4.i; A.413.D.4.i; A.414.D.4.i; A.415.D.4.i; A.416.D.4.i; A.417.D.4.i;
A.418.D.4.i; A.419.D.4.i; A.420.D.4.i; A.421.D.4.i; A.422.D.4.i; A.423.D.4.i;
A.424.D.4.i; A.425.D.4.i; A.426.D.4.i; A.427.D.4.i; A.428.D.4.i; A.429.D.4.i;
A.430.D.4.i; A.431.D.4.i; A.432.D.4.i; A.433.D.4.i; A.434.D.4.i; A.435.D.4.i;
A.436.D.4.i; A.437.D.4.i; A.438.D.4.i; A.439.D.4.i; A.440.D.4.i; A.441.D.4.i;
A.442.D.4.i; A.443.D.4.i; A.444.D.4.i; A.445.D.4.i; A.446.D.4.i; A.447.D.4.i;
A.448.D.4.i; A.449.D.4.i; A.450.D.4.i; A.451.D.4.i; A.452.D.4.i; A.453.D.4.i;
A.454.D.4.i; A.455.D.4.i; A.456.D.4.i; A.457.D.4.i; A.458.D.4.i; A.459.D.4.i;
A.460.D.4.i; A.461.D.4.i; A.462.D.4.i; A.463.D.4.i; A.464.D.4.i; A.465.D.4.i;
A.466.D.4.i; A.467.D.4.i; A.468.D.4.i; A.469.D.4.i; A.470.D.4.i; A.471.D.4.i;
A.472.D.4.i; A.473.D.4.i; A.474.D.4.i; A.475.D.4.i; A.476.D.4.i; A.477.D.4.i;
A.478.D.4.i; A.479.D.4.i; A.480.D.4.i; A.481.D.4.i; A.482.D.4.i; A.483.D.4.i;
A.484.D.4.i; A.485.D.4.i; A.486.D.4.i; A.487.D.4.i; A.488.D.4.i; A.489.D.4.i;
A.490.D.4.i; A.491.D.4.i; A.492.D.4.i; A.493.D.4.i; A.494.D.4.i; A.495.D.4.i;
A.496.D.4.i; A.497.D.4.i; A.498.D.4.i; A.499.D.4.i; A.500.D.4.i; A.501.D.4.i;
A.502.D.4.i; A.503.D.4.i; A.504.D.4.i; A.505.D.4.i; A.506.D.4.i; A.507.D.4.i;
A.508.D.4.i; A.509.D.4.i; A.510.D.4.i; A.511.D.4.i; A.512.D.4.i; A.512.D.4.i;
A.513.D.4.i; A.514.D.4.i; A.515.D.4.i; A.516.D.4.i; A.517.D.4.i; A.518.D.4.i;
A.519.D.4.i; A.520.D.4.i; A.521.D.4.i; A.522.D.4.i; A.523.D.4.i; A.524.D.4.i;
A.525.D.4.i; A.526.D.4.i; A.527.D.4.i; A.528.D.4.i; A.529.D.4.i; A.530.D.4.i;
A.531.D.4.i; A.532.D.4.i; A.533.D.4.i; A.534.D.4.i; A.535.D.4.i; A.536.D.4.i;
A.537.D.4.i; A.538.D.4.i; A.539.D.4.i; A.540.D.4.i; A.541.D.4.i; A.542.D.4.i;
A.543.D.4.i; A.544.D.4.i; A.545.D.4.i; A.546.D.4.i; A.547.D.4.i; A.548.D.4.i;
A.549.D.4.i; A.550.D.4.i; A.551.D.4.i; A.552.D.4.i; A.553.D.4.i; A.554.D.4.i;
A.555.D.4.i; A.556.D.4.i; A.557.D.4.i; A.558.D.4.i; A.559.D.4.i; A.560.D.4.i;
A.561.D.4.i; A.562.D.4.i; A.563.D.4.i; A.564.D.4.i; A.565.D.4.i; A.566.D.4.i;
A.567.D.4.i; A.568.D.4.i; A.569.D.4.i; A.570.D.4.i; A.571.D.4.i; A.572.D.4.i;
A.573.D.4.i; A.574.D.4.i; A.575.D.4.i; A.576.D.4.i; A.577.D.4.i; A.578.D.4.i;
A.579.D.4.i; A.580.D.4.i; A.581.D.4.i; A.582.D.4.i; A.583.D.4.i; A.584.D.4.i;
A.585.D.4.i; A.586.D.4.i; A.587.D.4.i; A.588.D.4.i; A.589.D.4.i; A.590.D.4.i;
A.591.D.4.i; A.592.D.4.i; A.593.D.4.i; A.594.D.4.i; A.595.D.4.i; A.596.D.4.i;
A.597.D.4.i; A.598.D.4.i; A.599.D.4.i; A.600.D.4.i; A.601.D.4.i; A.602.D.4.i;
A.603.D.4.i; A.604.D.4.i; A.605.D.4.i; A.606.D.4.i; A.607.D.4.i; A.608.D.4.i;
A.609.D.4.i; A.610.D.4.i; A.611.D.4.i; A.612.D.4.i; A.613.D.4.i; A.614.D.4.i;
A.615.D.4.i; A.616.D.4.i; A.617.D.4.i; A.618.D.4.i; A.619.D.4.i; A.620.D.4.i;
A.621.D.4.i; A.622.D.4.i; A.623.D.4.i; A.624.D.4.i; A.625.D.4.i; A.626.D.4.i;
A.627.D.4.i; A.628.D.4.i; A.629.D.4.i; A.630.D.4.i; A.631.D.4.i; A.632.D.4.i;
A.633.D.4.i; A.634.D.4.i; A.635.D.4.i; A.636.D.4.i; A.637.D.4.i; A.638.D.4.i;
A.639.D.4.i; A.640.D.4.i; A.641.D.4.i; A.642.D.4.i; A.643.D.4.i; A.644.D.4.i;
A.645.D.4.i; A.646.D.4.i; A.647.D.4.i; A.648.D.4.i; A.649.D.4.i; A.650.D.4.i;
A.651.D.4.i; A.652.D.4.i; A.653.D.4.i; A.654.D.4.i; A.655.D.4.i; A.656.D.4.i;
A.657.D.4.i; A.658.D.4.i; A.659.D.4.i; A.660.D.4.i; A.2.D.11.i; A.3.D.11.i;
A.4.D.11.i; A.5.D.11.i; A.6.D.11.i; A.7.D.11.i; A.9.D.11.i; A.10.D.11.i; A.15.D.11.i;
A.100.D.11.i; A.101.D.11.i; A.102.D.11.i; A.103.D.11.i; A.104.D.11.i; A.105.D.11.i;
A.106.D.11.i; A.107.D.11.i; A.108.D.11.i; A.109.D.11.i; A.110.D.11.i; A.111.D.11.i;
A.112.D.11.i; A.113.D.11.i; A.114.D.11.i; A.115.D.11.i; A.116.D.11.i; A.117.D.11.i;
A.118.D.11.i; A.119.D.11.i; A.120.D.11.i; A.121.D.11.i; A.122.D.11.i; A.123.D.11.i;
A.124.D.11.i; A.125.D.11.i; A.126.D.11.i; A.127.D.11.i; A.128.D.11.i; A.129.D.11.i;
A.130.D.11.i; A.131.D.11.i; A.132.D.11.i; A.133.D.11.i; A.134.D.11.i; A.135.D.11.i;
A.136.D.11.i; A.137.D.11.i; A.138.D.11.i; A.139.D.11.i; A.140.D.11.i; A.141.D.11.i;
A.142.D.11.i; A.143.D.11.i; A.144.D.11.i; A.145.D.11.i; A.146.D.11.i; A.147.D.11.i;
A.148.D.11.i; A.149.D.11.i; A.150.D.11.i; A.151.D.11.i; A.152.D.11.i; A.153.D.11.i;
A.154.D.11.i; A.155.D.11.i; A.156.D.11.i; A.157.D.11.i; A.158.D.11.i; A.159.D.11.i;
A.160.D.11.i; A.161.D.11.i; A.162.D.11.i; A.163.D.11.i; A.164.D.11.i; A.165.D.11.i;
A.166.D.11.i; A.167.D.11.i; A.168.D.11.i; A.169.D.11.i; A.170.D.11.i; A.171.D.11.i;
A.172.D.11.i; A.173.D.11.i; A.174.D.11.i; A.175.D.11.i; A.176.D.11.i; A.177.D.11.i;
A.178.D.11.i; A.179.D.11.i; A.180.D.11.i; A.181.D.11.i; A.182.D.11.i; A.183.D.11.i;
A.184.D.11.i; A.185.D.11.i; A.186.D.11.i; A.187.D.11.i; A.188.D.11.i; A.189.D.11.i;
A.190.D.11.i; A.191.D.11.i; A.192.D.11.i; A.193.D.11.i; A.194.D.11.i; A.195.D.11.i;
A.196.D.11.i; A.197.D.11.i; A.198.D.11.i; A.199.D.11.i; A.200.D.11.i; A.201.D.11.i;
A.202.D.11.i; A.203.D.11.i; A.204.D.11.i; A.205.D.11.i; A.206.D.11.i; A.207.D.11.i;
A.208.D.11.i; A.209.D.11.i; A.210.D.11.i; A.211.D.11.i; A.212.D.11.i; A.213.D.11.i;
A.214.D.11.i; A.215.D.11.i; A.216.D.11.i; A.217.D.11.i; A.218.D.11.i; A.219.D.11.i;
A.220.D.11.i; A.221.D.11.i; A.222.D.11.i; A.223.D.11.i; A.224.D.11.i; A.225.D.11.i;
A.226.D.11.i; A.227.D.11.i; A.228.D.11.i; A.229.D.11.i; A.230.D.11.i; A.231.D.11.i;
A.232.D.11.i; A.233.D.11.i; A.234.D.11.i; A.235.D.11.i; A.236.D.11.i; A.237.D.11.i;
A.238.D.11.i; A.239.D.11.i; A.240.D.11.i; A.241.D.11.i; A.242.D.11.i; A.243.D.11.i;
A.244.D.11.i; A.245.D.11.i; A.246.D.11.i; A.247.D.11.i; A.248.D.11.i; A.249.D.11.i;
A.250.D.11.i; A.251.D.11.i; A.252.D.11.i; A.253.D.11.i; A.254.D.11.i; A.255.D.11.i;
A.256.D.11.i; A.257.D.11.i; A.258.D.11.i; A.259.D.11.i; A.260.D.11.i; A.261.D.11.i;
A.262.D.11.i; A.263.D.11.i; A.264.D.11.i; A.265.D.11.i; A.266.D.11.i; A.267.D.11.i;
A.268.D.11.i; A.269.D.11.i; A.270.D.11.i; A.271.D.11.i; A.272.D.11.i; A.273.D.11.i;
A.274.D.11.i; A.275.D.11.i; A.276.D.11.i; A.277.D.11.i; A.278.D.11.i; A.279.D.11.i;
A.280.D.11.i; A.281.D.11.i; A.282.D.11.i; A.283.D.11.i; A.284.D.11.i; A.285.D.11.i;
A.286.D.11.i; A.287.D.11.i; A.288.D.11.i; A.289.D.11.i; A.290.D.11.i; A.291.D.11.i;
A.292.D.11.i; A.293.D.11.i; A.294.D.11.i; A.295.D.11.i; A.296.D.11.i; A.297.D.11.i;
A.298.D.11.i; A.299.D.11.i; A.300.D.11.i; A.301.D.11.i; A.302.D.11.i; A.303.D.11.i;
A.304.D.11.i; A.305.D.11.i; A.306.D.11.i; A.307.D.11.i; A.308.D.11.i; A.309.D.11.i;
A.310.D.11.i; A.311.D.11.i; A.312.D.11.i; A.313.D.11.i; A.314.D.11.i; A.315.D.11.i;
A.316.D.11.i; A.317.D.11.i; A.318.D.11.i; A.319.D.11.i; A.320.D.11.i; A.321.D.11.i;
A.323.D.11.i; A.324.D.11.i; A.325.D.11.i; A.326.D.11.i; A.327.D.11.i; A.328.D.11.i;
A.329.D.11.i; A.330.D.11.i; A.331.D.11.i; A.332.D.11.i; A.333.D.11.i; A.334.D.11.i;
A.335.D.11.i; A.336.D.11.i; A.337.D.11.i; A.338.D.11.i; A.339.D.11.i; A.340.D.11.i;
A.341.D.11.i; A.342.D.11.i; A.343.D.11.i; A.344.D.11.i; A.345.D.11.i; A.346.D.11.i;
A.347.D.11.i; A.348.D.11.i; A.349.D.11.i; A.350.D.11.i; A.351.D.11.i; A.352.D.11.i;
A.353.D.11.i; A.354.D.11.i; A.355.D.11.i; A.356.D.11.i; A.357.D.11.i; A.358.D.11.i;
A.359.D.11.i; A.360.D.11.i; A.361.D.11.i; A.362.D.11.i; A.363.D.11.i; A.364.D.11.i;
A.365.D.11.i; A.366.D.11.i; A.367.D.11.i; A.368.D.11.i; A.369.D.11.i; A.370.D.11.i;
A.371.D.11.i; A.372.D.11.i; A.373.D.11.i; A.374.D.11.i; A.375.D.11.i; A.376.D.11.i;
A.377.D.11.i; A.378.D.11.i; A.379.D.11.i; A.380.D.11.i; A.381.D.11.i; A.382.D.11.i;
A.383.D.11.i; A.384.D.11.i; A.385.D.11.i; A.386.D.11.i; A.387.D.11.i; A.388.D.11.i;
A.389.D.11.i; A.390.D.11.i; A.391.D.11.i; A.392.D.11.i; A.393.D.11.i; A.394.D.11.i;
A.395.D.11.i; A.396.D.11.i; A.397.D.11.i; A.398.D.11.i; A.399.D.11.i; A.400.D.11.i;
A.401.D.11.i; A.402.D.11.i; A.403.D.11.i; A.404.D.11.i; A.405.D.11.i; A.406.D.11.i;
A.407.D.11.i; A.408.D.11.i; A.409.D.11.i; A.410.D.11.i; A.411.D.11.i; A.412.D.11.i;
A.413.D.11.i; A.414.D.11.i; A.415.D.11.i; A.416.D.11.i; A.417.D.11.i; A.418.D.11.i;
A.419.D.11.i; A.420.D.11.i; A.421.D.11.i; A.422.D.11.i; A.423.D.11.i; A.424.D.11.i;
A.425.D.11.i; A.426.D.11.i; A.427.D.11.i; A.428.D.11.i; A.429.D.11.i; A.430.D.11.i;
A.431.D.11.i; A.432.D.11.i; A.433.D.11.i; A.434.D.11.i; A.435.D.11.i; A.436.D.11.i;
A.437.D.11.i; A.438.D.11.i; A.439.D.11.i; A.440.D.11.i; A.441.D.11.i; A.442.D.11.i;
A.443.D.11.i; A.444.D.11.i; A.445.D.11.i; A.446.D.11.i; A.447.D.11.i; A.448.D.11.i;
A.449.D.11.i; A.450.D.11.i; A.451.D.11.i; A.452.D.11.i; A.453.D.11.i; A.454.D.11.i;
A.455.D.11.i; A.456.D.11.i; A.457.D.11.i; A.458.D.11.i; A.459.D.11.i; A.460.D.11.i;
A.461.D.11.i; A.462.D.11.i; A.463.D.11.i; A.464.D.11.i; A.465.D.11.i; A.466.D.11.i;
A.467.D.11.i; A.468.D.11.i; A.469.D.11.i; A.470.D.11.i; A.471.D.11.i; A.472.D.11.i;
A.473.D.11.i; A.474.D.11.i; A.475.D.11.i; A.476.D.11.i; A.477.D.11.i; A.478.D.11.i;
A.479.D.11.i; A.480.D.11.i; A.481.D.11.i; A.482.D.11.i; A.483.D.11.i; A.484.D.11.i;
A.485.D.11.i; A.486.D.11.i; A.487.D.11.i; A.488.D.11.i; A.489.D.11.i; A.490.D.11.i;
A.491.D.11.i; A.492.D.11.i; A.493.D.11.i; A.494.D.11.i; A.495.D.11.i; A.496.D.11.i;
A.497.D.11.i; A.498.D.11.i; A.499.D.11.i; A.500.D.11.i; A.501.D.11.i; A.502.D.11.i;
A.503.D.11.i; A.504.D.11.i; A.505.D.11.i; A.506.D.11.i; A.507.D.11.i; A.508.D.11.i;
A.509.D.11.i; A.510.D.11.i; A.511.D.11.i; A.512.D.11.i; A.512.D.11.i; A.513.D.11.i;
A.514.D.11.i; A.515.D.11.i; A.516.D.11.i; A.517.D.11.i; A.518.D.11.i; A.519.D.11.i;
A.520.D.11.i; A.521.D.11.i; A.522.D.11.i; A.523.D.11.i; A.524.D.11.i; A.525.D.11.i;
A.526.D.11.i; A.527.D.11.i; A.528.D.11.i; A.529.D.11.i; A.530.D.11.i; A.531.D.11.i;
A.532.D.11.i; A.533.D.11.i; A.534.D.11.i; A.535.D.11.i; A.536.D.11.i; A.537.D.11.i;
A.538.D.11.i; A.539.D.11.i; A.540.D.11.i; A.541.D.11.i; A.542.D.11.i; A.543.D.11.i;
A.544.D.11.i; A.545.D.11.i; A.546.D.11.i; A.547.D.11.i; A.548.D.11.i; A.549.D.11.i;
A.550.D.11.i; A.551.D.11.i; A.552.D.11.i; A.553.D.11.i; A.554.D.11.i; A.555.D.11.i;
A.556.D.11.i; A.557.D.11.i; A.558.D.11.i; A.559.D.11.i; A.560.D.11.i; A.561.D.11.i;
A.562.D.11.i; A.563.D.11.i; A.564.D.11.i; A.565.D.11.i; A.566.D.11.i; A.567.D.11.i;
A.568.D.11.i; A.569.D.11.i; A.570.D.11.i; A.571.D.11.i; A.572.D.11.i; A.573.D.11.i;
A.574.D.11.i; A.575.D.11.i; A.576.D.11.i; A.577.D.11.i; A.578.D.11.i; A.579.D.11.i;
A.580.D.11.i; A.581.D.11.i; A.582.D.11.i; A.583.D.11.i; A.584.D.11.i; A.585.D.11.i;
A.586.D.11.i; A.587.D.11.i; A.588.D.11.i; A.589.D.11.i; A.590.D.11.i; A.591.D.11.i;
A.592.D.11.i; A.593.D.11.i; A.594.D.11.i; A.595.D.11.i; A.596.D.11.i; A.597.D.11.i;
A.598.D.11.i; A.599.D.11.i; A.600.D.11.i; A.601.D.11.i; A.602.D.11.i; A.603.D.11.i;
A.604.D.11.i; A.605.D.11.i; A.606.D.11.i; A.607.D.11.i; A.608.D.11.i; A.609.D.11.i;
A.610.D.11.i; A.611.D.11.i; A.612.D.11.i; A.613.D.11.i; A.614.D.11.i; A.615.D.11.i;
A.616.D.11.i; A.617.D.11.i; A.618.D.11.i; A.619.D.11.i; A.620.D.11.i; A.621.D.11.i;
A.622.D.11.i; A.623.D.11.i; A.624.D.11.i; A.625.D.11.i; A.626.D.11.i; A.627.D.11.i;
A.628.D.11.i; A.629.D.11.i; A.630.D.11.i; A.631.D.11.i; A.632.D.11.i; A.633.D.11.i;
A.634.D.11.i; A.635.D.11.i; A.636.D.11.i; A.637.D.11.i; A.638.D.11.i; A.639.D.11.i;
A.640.D.11.i; A.641.D.11.i; A.642.D.11.i; A.643.D.11.i; A.644.D.11.i; A.645.D.11.i;
A.646.D.11.i; A.647.D.11.i; A.648.D.11.i; A.649.D.11.i; A.650.D.11.i; A.651.D.11.i;
A.652.D.11.i; A.653.D.11.i; A.654.D.11.i; A.655.D.11.i; A.656.D.11.i; A.657.D.11.i;
A.658.D.11.i; A.659.D.11.i; A.660.D.11.i; A.2.E.4.i; A.3.E.4.i; A.4.E.4.i; A.5.E.4.i;
A.6.E.4.i; A.7.E.4.i; A.9.E.4.i; A.10.E.4.i; A.15.E.4.i; A.100.E.4.i; A.101.E.4.i;
A.102.E.4.i; A.103.E.4.i; A.104.E.4.i; A.105.E.4.i; A.106.E.4.i; A.107.E.4.i;
A.108.E.4.i; A.109.E.4.i; A.110.E.4.i; A.111.E.4.i; A.112.E.4.i; A.113.E.4.i;
A.114.E.4.i; A.115.E.4.i; A.116.E.4.i; A.117.E.4.i; A.118.E.4.i; A.119.E.4.i;
A.120.E.4.i; A.121.E.4.i; A.122.E.4.i; A.123.E.4.i; A.124.E.4.i; A.125.E.4.i;
A.126.E.4.i; A.127.E.4.i; A.128.E.4.i; A.129.E.4.i; A.130.E.4.i; A.131.E.4.i;
A.132.E.4.i; A.133.E.4.i; A.134.E.4.i; A.135.E.4.i; A.136.E.4.i; A.137.E.4.i;
A.138.E.4.i; A.139.E.4.i; A.140.E.4.i; A.141.E.4.i; A.142.E.4.i; A.143.E.4.i;
A.144.E.4.i; A.145.E.4.i; A.146.E.4.i; A.147.E.4.i; A.148.E.4.i; A.149.E.4.i;
A.150.E.4.i; A.151.E.4.i; A.152.E.4.i; A.153.E.4.i; A.154.E.4.i; A.155.E.4.i;
A.156.E.4.i; A.157.E.4.i; A.158.E.4.i; A.159.E.4.i; A.160.E.4.i; A.161.E.4.i;
A.162.E.4.i; A.163.E.4.i; A.164.E.4.i; A.165.E.4.i; A.166.E.4.i; A.167.E.4.i;
A.168.E.4.i; A.169.E.4.i; A.170.E.4.i; A.171.E.4.i; A.172.E.4.i; A.173.E.4.i;
A.174.E.4.i; A.175.E.4.i; A.176.E.4.i; A.177.E.4.i; A.178.E.4.i; A.179.E.4.i;
A.180.E.A.i; A.181.E.4.i; A.182.E.4.i; A.183.E.4.i; A.184.E.4.i; A.185.E.4.i;
A.186.E.4.i; A.187.E.4.i; A.188.E.4.i; A.189.E.4.i; A.190.E.4.i; A.191.E.4.i;
A.192.E.4.i; A.193.E.4.i; A.194.E.4.i; A.195.E.4.i; A.196.E.4.i; A.197.E.4.i;
A.198.E.4.i; A.199.E.4.i; A.200.E.4.i; A.201.E.4.i; A.202.E.4.i; A.203.E.4.i;
A.204.E.4.i; A.205.E.4.i; A.206.E.4.i; A.207.E.4.i; A.208.E.4.i; A.209.E.4.i;
A.210.E.4.i; A.211.E.4.i; A.212.E.4.i; A.213.E.4.i; A.214.E.4.i; A.215.E.4.i;
A.216.E.4.i; A.217.E.4.i; A.218.E.4.i; A.219.E.4.i; A.220.E.4.i; A.221.E.4.i;
A.222.E.4.i; A.223.E.4.i; A.224.E.4.i; A.225.E.4.i; A.226.E.4.i; A.227.E.4.i;
A.228.E.4.i; A.229.E.4.i; A.230.E.4.i; A.231.E.4.i; A.232.E.4.i; A.233.E.4.i;
A.234.E.4.i; A.235.E.4.i; A.236.E.4.i; A.237.E.4.i; A.238.E.4.i; A.239.E.4.i;
A.240.E.4.i; A.241.E.4.i; A.242.E.4.i; A.243.E.4.i; A.244.E.4.i; A.245.E.4.i;
A.246.E.4.i; A.247.E.4.i; A.248.E.4.i; A.249.E.4.i; A.250.E.4.i; A.251.E.4.i;
A.252.E.4.i; A.253.E.4.i; A.254.E.4.i; A.255.E.4.i; A.256.E.4.i; A.257.E.4.i;
A.258.E.4.i; A.259.E.4.i; A.260.E.4.i; A.261.E.4.i; A.262.E.4.i; A.263.E.4.i;
A.264.E.4.i; A.265.E.4.i; A.266.E.4.i; A.267.E.4.i; A.268.E.4.i; A.269.E.4.i;
A.270.E.4.i; A.271.E.4.i; A.272.E.4.i; A.273.E.4.i; A.274.E.4.i; A.275.E.4.i;
A.276.E.4.i; A.277.E.4.i; A.278.E.4.i; A.279.E.4.i; A.280.E.4.i; A.281.E.4.i;
A.282.E.4.i; A.283.E.4.i; A.284.E.4.i; A.285.E.4.i; A.286.E.4.i; A.287.E.4.i;
A.288.E.4.i; A.289.E.4.i; A.290.E.4.i; A.291.E.4.i; A.292.E.4.i; A.293.E.4.i;
A.294.E.4.i; A.295.E.4.i; A.296.E.4.i; A.297.E.4.i; A.298.E.4.i; A.299.E.4.i;
A.300.E.4.i; A.301.E.4.i; A.302.E.4.i; A.303.E.4.i; A.304.E.4.i; A.305.E.4.i;
A.306.E.4.i; A.307.E.4.i; A.308.E.4.i; A.309.E.4.i; A.310.E.4.i; A.311.E.4.i;
A.312.E.4.i; A.313.E.4.i; A.314.E.4.i; A.315.E.4.i; A.316.E.4.i; A.317.E.4.i;
A.318.E.4.i; A.319.E.4.i; A.320.E.4.i; A.321.E.4.i; A.323.E.4.i; A.324.E.4.i;
A.325.E.4.i; A.326.E.4.i; A.327.E.4.i; A.328.E.4.i; A.329.E.4.i; A.330.E.4.i;
A.331.E.4.i; A.332.E.4.i; A.333.E.4.i; A.334.E.4.i; A.335.E.4.i; A.336.E.4.i;
A.337.E.4.i; A.338.E.4.i; A.339.E.4.i; A.340.E.4.i; A.341.E.4.i; A.342.E.4.i;
A.343.E.4.i; A.344.E.4.i; A.345.E.4.i; A.346.E.4.i; A.347.E.4.i; A.348.E.4.i;
A.349.E.4.i; A.350.E.4.i; A.351.E.4.i; A.352.E.4.i; A.353.E.4.i; A.354.E.4.i;
A.355.E.4.i; A.356.E.4.i; A.357.E.4.i; A.358.E.4.i; A.359.E.4.i; A.360.E.4.i;
A.361.E.4.i; A.362.E.4.i; A.363.E.4.i; A.364.E.4.i; A.365.E.4.i; A.366.E.4.i;
A.367.E.4.i; A.368.E.4.i; A.369.E.4.i; A.370.E.4.i; A.371.E.4.i; A.372.E.4.i;
A.373.E.4.i; A.374.E.4.i; A.375.E.4.i; A.376.E.4.i; A.377.E.4.i; A.378.E.4.i;
A.379.E.4.i; A.380.E.4.i; A.381.E.4.i; A.382.E.4.i; A.383.E.4.i; A.384.E.4.i;
A.385.E.4.i; A.386.E.4.i; A.387.E.4.i; A.388.E.4.i; A.389.E.4.i; A.390.E.4.i;
A.391.E.4.i; A.392.E.4.i; A.393.E.4.i; A.394.E.4.i; A.395.E.4.i; A.396.E.4.i;
A.397.E.4.i; A.398.E.4.i; A.399.E.4.i; A.400.E.4.i; A.401.E.4.i; A.402.E.4.i;
A.403.E.4.i; A.404.E.4.i; A.405.E.4.i; A.406.E.4.i; A.407.E.4.i; A.408.E.4.i;
A.409.E.4.i; A.410.E.4.i; A.411.E.4.i; A.412.E.4.i; A.413.E.4.i; A.414.E.4.i;
A.415.E.4.i; A.416.E.4.i; A.417.E.4.i; A.418.E.4.i; A.419.E.4.i; A.420.E.4.i;
A.421.E.4.i; A.422.E.4.i; A.423.E.4.i; A.424.E.4.i; A.425.E.4.i; A.426.E.4.i;
A.427.E.4.i; A.428.E.4.i; A.429.E.4.i; A.430.E.4.i; A.431.E.4.i; A.432.E.4.i;
A.433.E.4.i; A.434.E.4.i; A.435.E.4.i; A.436.E.4.i; A.437.E.4.i; A.438.E.4.i;
A.439.E.4.i; A.440.E.4.i; A.441.E.4.i; A.442.E.4.i; A.443.E.4.i; A.444.E.4.i;
A.445.E.4.i; A.446.E.4.i; A.447.E.4.i; A.448.E.4.i; A.449.E.4.i; A.450.E.4.i;
A.451.E.4.i; A.452.E.4.i; A.453.E.4.i; A.454.E.4.i; A.455.E.4.i; A.456.E.4.i;
A.457.E.4.i; A.458.E.4.i; A.459.E.4.i; A.460.E.4.i; A.461.E.4.i; A.462.E.4.i;
A.463.E.4.i; A.464.E.4.i; A.465.E.4.i; A.466.E.4.i; A.467.E.4.i; A.468.E.4.i;
A.469.E.4.i; A.470.E.4.i; A.471.E.4.i; A.472.E.4.i; A.473.E.4.i; A.474.E.4.i;
A.475.E.4.i; A.476.E.4.i; A.477.E.4.i; A.478.E.4.i; A.479.E.4.i; A.480.E.4.i;
A.481.E.4.i; A.482.E.4.i; A.483.E.4.i; A.484.E.4.i; A.485.E.4.i; A.486.E.4.i;
A.487.E.4.i; A.488.E.4.i; A.489.E.4.i; A.490.E.4.i; A.491.E.4.i; A.492.E.4.i;
A.493.E.4.i; A.494.E.4.i; A.495.E.4.i; A.496.E.4.i; A.497.E.4.i; A.498.E.4.i;
A.499.E.4.i; A.500.E.4.i; A.501.E.4.i; A.502.E.4.i; A.503.E.4.i; A.504.E.4.i;
A.505.E.4.i; A.506.E.4.i; A.507.E.4.i; A.508.E.4.i; A.509.E.4.i; A.510.E.4.i;
A.511.E.4.i; A.512.E.4.i; A.512.E.4.i; A.513.E.4.i; A.514.E.4.i; A.515.E.4.i;
A.516.E.4.i; A.517.E.4.i; A.518.E.4.i; A.519.E.4.i; A.520.E.4.i; A.521.E.4.i;
A.522.E.4.i; A.523.E.4.i; A.524.E.4.i; A.525.E.4.i; A.526.E.4.i; A.527.E.4.i;
A.528.E.4.i; A.529.E.4.i; A.530.E.4.i; A.531.E.4.i; A.532.E.4.i; A.533.E.4.i;
A.534.E.4.i; A.535.E.4.i; A.536.E.4.i; A.537.E.4.i; A.538.E.4.i; A.539.E.4.i;
A.540.E.4.i; A.541.E.4.i; A.542.E.4.i; A.543.E.4.i; A.544.E.4.i; A.545.E.4.i;
A.546.E.4.i; A.547.E.4.i; A.548.E.4.i; A.549.E.4.i; A.550.E.4.i; A.551.E.4.i;
A.552.E.4.i; A.553.E.4.i; A.554.E.4.i; A.555.E.4.i; A.556.E.4.i; A.557.E.4.i;
A.558.E.4.i; A.559.E.4.i; A.560.E.4.i; A.561.E.4.i; A.562.E.4.i; A.563.E.4.i;
A.564.E.4.i; A.565.E.4.i; A.566.E.4.i; A.567.E.4.i; A.568.E.4.i; A.569.E.4.i;
A.570.E.4.i; A.571.E.4.i; A.572.E.4.i; A.573.E.4.i; A.574.E.4.i; A.575.E.4.i;
A.576.E.4.i; A.577.E.4.i; A.578.E.4.i; A.579.E.4.i; A.580.E.4.i; A.581.E.4.i;
A.582.E.4.i; A.583.E.4.i; A.584.E.4.i; A.585.E.4.i; A.586.E.4.i; A.587.E.4.i;
A.588.E.4.i; A.589.E.4.i; A.590.E.4.i; A.591.E.4.i; A.592.E.4.i; A.593.E.4.i;
A.594.E.4.i; A.595.E.4.i; A.596.E.4.i; A.597.E.4.i; A.598.E.4.i; A.599.E.4.i;
A.600.E.4.i; A.601.E.4.i; A.602.E.4.i; A.603.E.4.i; A.604.E.4.i; A.605.E.4.i;
A.606.E.4.i; A.607.E.4.i; A.608.E.4.i; A.609.E.4.i; A.610.E.4.i; A.611.E.4.i;
A.612.E.4.i; A.613.E.4.i; A.614.E.4.i; A.615.E.4.i; A.616.E.4.i; A.617.E.4.i;
A.618.E.4.i; A.619.E.4.i; A.620.E.4.i; A.621.E.4.i; A.622.E.4.i; A.623.E.4.i;
A.624.E.4.i; A.625.E.4.i; A.626.E.4.i; A.627.E.4.i; A.628.E.4.i; A.629.E.4.i;
A.630.E.4.i; A.631.E.4.i; A.632.E.4.i; A.633.E.4.i; A.634.E.4.i; A.635.E.4.i;
A.636.E.4.i; A.637.E.4.i; A.638.E.4.i; A.639.E.4.i; A.640.E.4.i; A.641.E.4.i;
A.642.E.4.i; A.643.E.4.i; A.644.E.4.i; A.645.E.4.i; A.646.E.4.i; A.647.E.4.i;
A.648.E.4.i; A.649.E.4.i; A.650.E.4.i; A.651.E.4.i; A.652.E.4.i; A.653.E.4.i;
A.654.E.4.i; A.655.E.4.i; A.656.E.4.i; A.657.E.4.i; A.658.E.4.i; A.659.E.4.i;
A.660.E.4.i; A.2.E.11.i; A.3.E.11.i; A.4.E.11.i; A.5.E.11.i; A.6.E.11.i; A.7.E.11.i;
A.9.E.11.i; A.10.E.11.i; A.15.E.11.i; A.100.E.11.i; A.101.E.11.i; A.102.E.11.i;
A.103.E.11.i; A.104.E.11.i; A.105.E.11.i; A.106.E.11.i; A.107.E.11.i; A.108.E.11.i;
A.109.E.11.i; A.110.E.11.i; A.111.E.11.i; A.112.E.11.i; A.113.E.11.i; A.114.E.11.i;
A.115.E.11.i; A.116.E.11.i; A.117.E.11.i; A.118.E.11.i; A.119.E.11.i; A.120.E.11.i;
A.121.E.11.i; A.122.E.11.i; A.123.E.11.i; A.124.E.11.i; A.125.E.11.i; A.126.E.11.i;
A.127.E.11.i; A.128.E.11.i; A.129.E.11.i; A.130.E.11.i; A.131.E.11.i; A.132.E.11.i;
A.133.E.11.i; A.134.E.11.i; A.135.E.11.i; A.136.E.11.i; A.137.E.11.i; A.138.E.11.i;
A.139.E.11.i; A.140.E.11.i; A.141.E.11.i; A.142.E.11.i; A.143.E.11.i; A.144.E.11.i;
A.145.E.11.i; A.146.E.11.i; A.147.E.11.i; A.148.E.11.i; A.149.E.11.i; A.150.E.11.i;
A.151.E.11.i; A.152.E.11.i; A.153.E.11.i; A.154.E.11.i; A.155.E.11.i; A.156.E.11.i;
A.157.E.11.i; A.158.E.11.i; A.159.E.11.i; A.160.E.11.i; A.161.E.11.i; A.162.E.11.i;
A.163.E.11.i; A.164.E.11.i; A.165.E.11.i; A.166.E.11.i; A.167.E.11.i; A.168.E.11.i;
A.169.E.11.i; A.170.E.11.i; A.171.E.11.i; A.172.E.11.i; A.173.E.11.i; A.174.E.11.i;
A.175.E.11.i; A.176.E.11.i; A.177.E.11.i; A.178.E.11.i; A.179.E.11.i; A.180.E.11.i;
A.181.E.11.i; A.182.E.11.i; A.183.E.11.i; A.184.E.11.i; A.185.E.11.i; A.186.E.11.i;
A.187.E.11.i; A.188.E.11.i; A.189.E.11.i; A.190.E.11.i; A.191.E.11.i; A.192.E.11.i;
A.193.E.11.i; A.194.E.11.i; A.195.E.11.i; A.196.E.11.i; A.197.E.11.i; A.198.E.11.i;
A.199.E.11.i; A.200.E.11.i; A.201.E.11.i; A.202.E.11.i; A.203.E.11.i; A.204.E.11.i;
A.205.E.11.i; A.206.E.11.i; A.207.E.11.i; A.208.E.11.i; A.209.E.11.i; A.210.E.11.i;
A.211.E.11.i; A.212.E.11.i; A.213.E.11.i; A.214.E.11.i; A.215.E.11.i; A.216.E.11.i;
A.217.E.11.i; A.218.E.11.i; A.219.E.11.i; A.220.E.11.i; A.221.E.11.i; A.222.E.11.i;
A.223.E.11.i; A.224.E.11.i; A.225.E.11.i; A.226.E.11.i; A.227.E.11.i; A.228.E.11.i;
A.229.E.11.i; A.230.E.11.i; A.231.E.11.i; A.232.E.11.i; A.233.E.11.i; A.234.E.11.i;
A.235.E.11.i; A.236.E.11.i; A.237.E.11.i; A.238.E.11.i; A.239.E.11.i; A.240.E.11.i;
A.241.E.11.i; A.242.E.11.i; A.243.E.11.i; A.244.E.11.i; A.245.E.11.i; A.246.E.11.i;
A.247.E.11.i; A.248.E.11.i; A.249.E.11.i; A.250.E.11.i; A.251.E.11.i; A.252.E.11.i;
A.253.E.11.i; A.254.E.11.i; A.255.E.11.i; A.256.E.11.i; A.257.E.11.i; A.258.E.11.i;
A.259.E.11.i; A.260.E.11.i; A.261.E.11.i; A.262.E.11.i; A.263.E.11.i; A.264.E.11.i;
A.265.E.11.i; A.266.E.11.i; A.267.E.11.i; A.268.E.11.i; A.269.E.11.i; A.270.E.11.i;
A.271.E.11.i; A.272.E.11.i; A.273.E.11.i; A.274.E.11.i; A.275.E.11.i; A.276.E.11.i;
A.277.E.11.i; A.278.E.11.i; A.279.E.11.i; A.280.E.11.i; A.281.E.11.i; A.282.E.11.i;
A.283.E.11.i; A.284.E.11.i; A.285.E.11.i; A.286.E.11.i; A.287.E.11.i; A.288.E.11.i;
A.289.E.11.i; A.290.E.11.i; A.291.E.11.i; A.292.E.11.i; A.293.E.11.i; A.294.E.11.i;
A.295.E.11.i; A.296.E.11.i; A.297.E.11.i; A.298.E.11.i; A.299.E.11.i; A.300.E.11.i;
A.301.E.11.i; A.302.E.11.i; A.303.E.11.i; A.304.E.11.i; A.305.E.11.i; A.306.E.11.i;
A.307.E.11.i; A.308.E.11.i; A.309.E.11.i; A.310.E.11.i; A.311.E.11.i; A.312.E.11.i;
A.313.E.11.i; A.314.E.11.i; A.315.E.11.i; A.316.E.11.i; A.317.E.11.i; A.318.E.11.i;
A.319.E.11.i; A.320.E.11.i; A.321.E.11.i; A.323.E.11.i; A.324.E.11.i; A.325.E.11.i;
A.326.E.11.i; A.327.E.11.i; A.328.E.11.i; A.329.E.11.i; A.330.E.11.i; A.331.E.11.i;
A.332.E.11.i; A.333.E.11.i; A.334.E.11.i; A.335.E.11.i; A.336.E.11.i; A.337.E.11.i;
A.338.E.11.i; A.339.E.11.i; A.340.E.11.i; A.341.E.11.i; A.342.E.11.i; A.343.E.11.i;
A.344.E.11.i; A.345.E.11.i; A.346.E.11.i; A.347.E.11.i; A.348.E.11.i; A.349.E.11.i;
A.350.E.11.i; A.351.E.11.i; A.352.E.11.i; A.353.E.11.i; A.354.E.11.i; A.355.E.11.i;
A.356.E.11.i; A.357.E.11.i; A.358.E.11.i; A.359.E.11.i; A.360.E.11.i; A.361.E.11.i;
A.362.E.11.i; A.363.E.11.i; A.364.E.11.i; A.365.E.11.i; A.366.E.11.i; A.367.E.11.i;
A.368.E.11.i; A.369.E.11.i; A.370.E.11.i; A.371.E.11.i; A.372.E.11.i; A.373.E.11.i;
A.374.E.11.i; A.375.E.11.i; A.376.E.11.i; A.377.E.11.i; A.378.E.11.i; A.379.E.11.i;
A.380.E.11.i; A.381.E.11.i; A.382.E.11.i; A.383.E.11.i; A.384.E.11.i; A.385.E.11.i;
A.386.E.11.i; A.387.E.11.i; A.388.E.11.i; A.389.E.11.i; A.390.E.11.i; A.391.E.11.i;
A.392.E.11.i; A.393.E.11.i; A.394.E.11.i; A.395.E.11.i; A.396.E.11.i; A.397.E.11.i;
A.398.E.11.i; A.399.E.11.i; A.400.E.11.i; A.401.E.11.i; A.402.E.11.i; A.403.E.11.i;
A.404.E.11.i; A.405.E.11.i; A.406.E.11.i; A.407.E.11.i; A.408.E.11.i; A.409.E.11.i;
A.410.E.11.i; A.411.E.11.i; A.412.E.11.i; A.413.E.11.i; A.414.E.11.i; A.415.E.11.i;
A.416.E.11.i; A.417.E.11.i; A.418.E.11.i; A.419.E.11.i; A.420.E.11.i; A.421.E.11.i;
A.422.E.11.i; A.423.E.11.i; A.424.E.11.i; A.425.E.11.i; A.426.E.11.i; A.427.E.11.i;
A.428.E.11.i; A.429.E.11.i; A.430.E.11.i; A.431.E.11.i; A.432.E.11.i; A.433.E.11.i;
A.434.E.11.i; A.435.E.11.i; A.436.E.11.i; A.437.E.11.i; A.438.E.11.i; A.439.E.11.i;
A.440.E.11.i; A.441.E.11.i; A.442.E.11.i; A.443.E.11.i; A.444.E.11.i; A.445.E.11.i;
A.446.E.11.i; A.447.E.11.i; A.448.E.11.i; A.449.E.11.i; A.450.E.11.i; A.451.E.11.i;
A.452.E.11.i; A.453.E.11.i; A.454.E.11.i; A.455.E.11.i; A.456.E.11.i; A.457.E.11.i;
A.458.E.11.i; A.459.E.11.i; A.460.E.11.i; A.461.E.11.i; A.462.E.11.i; A.463.E.11.i;
A.464.E.11.i; A.465.E.11.i; A.466.E.11.i; A.467.E.11.i; A.468.E.11.i; A.469.E.11.i;
A.470.E.11.i; A.471.E.11.i; A.472.E.11.i; A.473.E.11.i; A.474.E.11.i; A.475.E.11.i;
A.476.E.11.i; A.477.E.11.i; A.478.E.11.i; A.479.E.11.i; A.480.E.11.i; A.481.E.11.i;
A.482.E.11.i; A.483.E.11.i; A.484.E.11.i; A.485.E.11.i; A.486.E.11.i; A.487.E.11.i;
A.488.E.11.i; A.489.E.11.i; A.490.E.11.i; A.491.E.11.i; A.492.E.11.i; A.493.E.11.i;
A.494.E.11.i; A.495.E.11.i; A.496.E.11.i; A.497.E.11.i; A.498.E.11.i; A.499.E.11.i;
A.500.E.11.i; A.501.E.11.i; A.502.E.11.i; A.503.E.11.i; A.504.E.11.i; A.505.E.11.i;
A.506.E.11.i; A.507.E.11.i; A.508.E.11.i; A.509.E.11.i; A.510.E.11.i; A.511.E.11.i;
A.512.E.11.i; A.512.E.11.i; A.513.E.11.i; A.514.E.11.i; A.515.E.11.i; A.516.E.11.i;
A.517.E.11.i; A.518.E.11.i; A.519.E.11.i; A.520.E.11.i; A.521.E.11.i; A.522.E.11.i;
A.523.E.11.i; A.524.E.11.i; A.525.E.11.i; A.526.E.11.i; A.527.E.11.i; A.528.E.11.i;
A.529.E.11.i; A.530.E.11.i; A.531.E.11.i; A.532.E.11.i; A.533.E.11.i; A.534.E.11.i;
A.535.E.11.i; A.536.E.11.i; A.537.E.11.i; A.538.E.11.i; A.539.E.11.i; A.540.E.11.i;
A.541.E.11.i; A.542.E.11.i; A.543.E.11.i; A.544.E.11.i; A.545.E.11.i; A.546.E.11.i;
A.547.E.11.i; A.548.E.11.i; A.549.E.11.i; A.550.E.11.i; A.551.E.11.i; A.552.E.11.i;
A.553.E.11.i; A.554.E.11.i; A.555.E.11.i; A.556.E.11.i; A.557.E.11.i; A.558.E.11.i;
A.559.E.11.i; A.560.E.11.i; A.561.E.11.i; A.562.E.11.i; A.563.E.11.i; A.564.E.11.i;
A.565.E.11.i; A.566.E.11.i; A.567.E.11.i; A.568.E.11.i; A.569.E.11.i; A.570.E.11.i;
A.571.E.11.i; A.572.E.11.i; A.573.E.11.i; A.574.E.11.i; A.575.E.11.i; A.576.E.11.i;
A.577.E.11.i; A.578.E.11.i; A.579.E.11.i; A.580.E.11.i; A.581.E.11.i; A.582.E.11.i;
A.583.E.11.i; A.584.E.11.i; A.585.E.11.i; A.586.E.11.i; A.587.E.11.i; A.588.E.11.i;
A.589.E.11.i; A.590.E.11.i; A.591.E.11.i; A.592.E.11.i; A.593.E.11.i; A.594.E.11.i;
A.595.E.11.i; A.596.E.11.i; A.597.E.11.i; A.598.E.11.i; A.599.E.11.i; A.600.E.11.i;
A.601.E.11.i; A.602.E.11.i; A.603.E.11.i; A.604.E.11.i; A.605.E.11.i; A.606.E.11.i;
A.607.E.11.i; A.608.E.11.i; A.609.E.11.i; A.610.E.11.i; A.611.E.11.i; A.612.E.11.i;
A.613.E.11.i; A.614.E.11.i; A.615.E.11.i; A.616.E.11.i; A.617.E.11.i; A.618.E.11.i;
A.619.E.11.i; A.620.E.11.i; A.621.E.11.i; A.622.E.11.i; A.623.E.11.i; A.624.E.11.i;
A.625.E.11.i; A.626.E.11.i; A.627.E.11.i; A.628.E.11.i; A.629.E.11.i; A.630.E.11.i;
A.631.E.11.i; A.632.E.11.i; A.633.E.11.i; A.634.E.11.i; A.635.E.11.i; A.636.E.11.i;
A.637.E.11.i; A.638.E.11.i; A.639.E.11.i; A.640.E.11.i; A.641.E.11.i; A.642.E.11.i;
A.643.E.11.i; A.644.E.11.i; A.645.E.11.i; A.646.E.11.i; A.647.E.11.i; A.648.E.11.i;
A.649.E.11.i; A.650.E.11.i; A.651.E.11.i; A.652.E.11.i; A.653.E.11.i; A.654.E.11.i;
A.655.E.11.i; A.656.E.11.i; A.657.E.11.i; A.658.E.11.i; A.659.E.11.i; A.660.E.11.i;
A.2.F.4.i; A.3.F.4.i; A.4.F.4.i; A.5.F.4.i; A.6.F.4.i; A.7.F.4.i; A.9.F.4.i; A.10.F.4.i;
A.15.F.4.i; A.100.F.4.i; A.101.F.4.i; A.102.F.4.i; A.103.F.4.i; A.104.F.4.i;
A.105.F.4.i; A.106.F.4.i; A.107.F.4.i; A.108.F.4.i; A.109.F.4.i; A.110.F.4.i;
A.111.F.4.i; A.112.F.4.i; A.113.F.4.i; A.114.F.4.i; A.115.F.4.i; A.116.F.4.i;
A.117.F.4.i; A.118.F.4.i; A.119.F.4.i; A.120.F.4.i; A.121.F.4.i; A.122.F.4.i;
A.123.F.4.i; A.124.F.4.i; A.125.F.4.i; A.126.F.4.i; A.127.F.4.i; A.128.F.4.i;
A.129.F.4.i; A.130.F.4.i; A.131.F.4.i; A.132.F.4.i; A.133.F.4.i; A.134.F.4.i;
A.135.F.4.i; A.136.F.4.i; A.137.F.4.i; A.138.F.4.i; A.139.F.4.i; A.140.F.4.i;
A.141.F.4.i; A.142.F.4.i; A.143.F.4.i; A.144.F.4.i; A.145.F.4.i; A.146.F.4.i;
A.147.F.4.i; A.148.F.4.i; A.149.F.4.i; A.150.F.4.i; A.151.F.4.i; A.152.F.4.i;
A.153.F.4.i; A.154.F.4.i; A.155.F.4.i; A.156.F.4.i; A.157.F.4.i; A.158.F.4.i;
A.159.F.4.i; A.160.F.4.i; A.161.F.4.i; A.162.F.4.i; A.163.F.4.i; A.164.F.4.i;
A.165.F.4.i; A.166.F.4.i; A.167.F.4.i; A.168.F.4.i; A.169.F.4.i; A.170.F.4.i;
A.171.F.4.i; A.172.F.4.i; A.173.F.4.i; A.174.F.4.i; A.175.F.4.i; A.176.F.4.i;
A.177.F.4.i; A.178.F.4.i; A.179.F.4.i; A.180.F.4.i; A.181.F.4.i; A.182.F.4.i;
A.183.F.4.i; A.184.F.4.i; A.185.F.4.i; A.186.F.4.i; A.187.F.4.i; A.188.F.4.i;
A.189.F.4.i; A.190.F.4.i; A.191.F.4.i; A.192.F.4.i; A.193.F.4.i; A.194.F.4.i;
A.195.F.4.i; A.196.F.4.i; A.197.F.4.i; A.198.F.4.i; A.199.F.4.i; A.200.F.4.i;
A.201.F.4.i; A.202.F.4.i; A.203.F.4.i; A.204.F.4.i; A.205.F.4.i; A.206.F.4.i;
A.207.F.4.i; A.208.F.4.i; A.209.F.4.i; A.210.F.4.i; A.211.F.4.i; A.212.F.4.i;
A.213.F.4.i; A.214.F.4.i; A.215.F.4.i; A.216.F.4.i; A.217.F.4.i; A.218.F.4.i;
A.219.F.4.i; A.220.F.4.i; A.221.F.4.i; A.222.F.4.i; A.223.F.4.i; A.224.F.4.i;
A.225.F.4.i; A.226.F.4.i; A.227.F.4.i; A.228.F.4.i; A.229.F.4.i; A.230.F.4.i;
A.231.F.4.i; A.232.F.4.i; A.233.F.4.i; A.234.F.4.i; A.235.F.4.i; A.236.F.4.i;
A.237.F.4.i; A.238.F.4.i; A.239.F.4.i; A.240.F.4.i; A.241.F.4.i; A.242.F.4.i;
A.243.F.4.i; A.244.F.4.i; A.245.F.4.i; A.246.F.4.i; A.247.F.4.i; A.248.F.4.i;
A.249.F.4.i; A.250.F.4.i; A.251.F.4.i; A.252.F.4.i; A.253.F.4.i; A.254.F.4.i;
A.255.F.4.i; A.256.F.4.i; A.257.F.4.i; A.258.F.4.i; A.259.F.4.i; A.260.F.4.i;
A.261.F.4.i; A.262.F.4.i; A.263.F.4.i; A.264.F.4.i; A.265.F.4.i; A.266.F.4.i;
A.267.F.4.i; A.268.F.4.i; A.269.F.4.i; A.270.F.4.i; A.271.F.4.i; A.272.F.4.i;
A.273.F.4.i; A.274.F.4.i; A.275.F.4.i; A.276.F.4.i; A.277.F.4.i; A.278.F.4.i;
A.279.F.4.i; A.280.F.4.i; A.281.F.4.i; A.282.F.4.i; A.283.F.4.i; A.284.F.4.i;
A.285.F.4.i; A.286.F.4.i; A.287.F.4.i; A.288.F.4.i; A.289.F.4.i; A.290.F.4.i;
A.291.F.4.i; A.292.F.4.i; A.293.F.4.i; A.294.F.4.i; A.295.F.4.i; A.296.F.4.i;
A.297.F.4.i; A.298.F.4.i; A.299.F.4.i; A.300.F.4.i; A.301.F.4.i; A.302.F.4.i;
A.303.F.4.i; A.304.F.4.i; A.305.F.4.i; A.306.F.4.i; A.307.F.4.i; A.308.F.4.i;
A.309.F.4.i; A.310.F.4.i; A.311.F.4.i; A.312.F.4.i; A.313.F.4.i; A.314.F.4.i;
A.315.F.4.i; A.316.F.4.i; A.317.F.4.i; A.318.F.4.i; A.319.F.4.i; A.320.F.4.i;
A.321.F.4.i; A.323.F.4.i; A.324.F.4.i; A.325.F.4.i; A.326.F.4.i; A.327.F.4.i;
A.328.F.4.i; A.329.F.4.i; A.330.F.4.i; A.331.F.4.i; A.332.F.4.i; A.333.F.4.i;
A.334.F.4.i; A.335.F.4.i; A.336.F.4.i; A.337.F.4.i; A.338.F.4.i; A.339.F.4.i;
A.340.F.4.i; A.341.F.4.i; A.342.F.4.i; A.343.F.4.i; A.344.F.4.i; A.345.F.4.i;
A.346.F.4.i; A.347.F.4.i; A.348.F.4.i; A.349.F.4.i; A.350.F.4.i; A.351.F.4.i;
A.352.F.4.i; A.353.F.4.i; A.354.F.4.i; A.355.F.4.i; A.356.F.4.i; A.357.F.4.i;
A.358.F.4.i; A.359.F.4.i; A.360.F.4.i; A.361.F.4.i; A.362.F.4.i; A.363.F.4.i;
A.364.F.4.i; A.365.F.4.i; A.366.F.4.i; A.367.F.4.i; A.368.F.4.i; A.369.F.4.i;
A.370.F.4.i; A.371.F.4.i; A.372.F.4.i; A.373.F.4.i; A.374.F.4.i; A.375.F.4.i;
A.376.F.4.i; A.377.F.4.i; A.378.F.4.i; A.379.F.4.i; A.380.F.4.i; A.381.F.4.i;
A.382.F.4.i; A.383.F.4.i; A.384.F.4.i; A.385.F.4.i; A.386.F.4.i; A.387.F.4.i;
A.388.F.4.i; A.389.F.4.i; A.390.F.4.i; A.391.F.4.i; A.392.F.4.i; A.393.F.4.i;
A.394.F.4.i; A.395.F.4.i; A.396.F.4.i; A.397.F.4.i; A.398.F.4.i; A.399.F.4.i;
A.400.F.4.i; A.401.F.4.i; A.402.F.4.i; A.403.F.4.i; A.404.F.4.i; A.405.F.4.i;
A.406.F.4.i; A.407.F.4.i; A.408.F.4.i; A.409.F.4.i; A.410.F.4.i; A.411.F.4.i;
A.412.F.4.i; A.413.F.4.i; A.414.F.4.i; A.415.F.4.i; A.416.F.4.i; A.417.F.4.i;
A.418.F.4.i; A.419.F.4.i; A.420.F.4.i; A.421.F.4.i; A.422.F.4.i; A.423.F.4.i;
A.424.F.4.i; A.425.F.4.i; A.426.F.4.i; A.427.F.4.i; A.428.F.4.i; A.429.F.4.i;
A.430.F.4.i; A.431.F.4.i; A.432.F.4.i; A.433.F.4.i; A.434.F.4.i; A.435.F.4.i;
A.436.F.4.i; A.437.F.4.i; A.438.F.4.i; A.439.F.4.i; A.440.F.4.i; A.441.F.4.i;
A.442.F.4.i; A.443.F.4.i; A.444.F.4.i; A.445.F.4.i; A.446.F.4.i; A.447.F.4.i;
A.448.F.4.i; A.449.F.4.i; A.450.F.4.i; A.451.F.4.i; A.452.F.4.i; A.453.F.4.i;
A.454.F.4.i; A.455.F.4.i; A.456.F.4.i; A.457.F.4.i; A.458.F.4.i; A.459.F.4.i;
A.460.F.4.i; A.461.F.4.i; A.462.F.4.i; A.463.F.4.i; A.464.F.4.i; A.465.F.4.i;
A.466.F.4.i; A.467.F.4.i; A.468.F.4.i; A.469.F.4.i; A.470.F.4.i; A.471.F.4.i;
A.472.F.4.i; A.473.F.4.i; A.474.F.4.i; A.475.F.4.i; A.476.F.4.i; A.477.F.4.i;
A.478.F.4.i; A.479.F.4.i; A.480.F.4.i; A.481.F.4.i; A.482.F.4.i; A.483.F.4.i;
A.484.F.4.i; A.485.F.4.i; A.486.F.4.i; A.487.F.4.i; A.488.F.4.i; A.489.F.4.i;
A.490.F.4.i; A.491.F.4.i; A.492.F.4.i; A.493.F.4.i; A.494.F.4.i; A.495.F.4.i;
A.496.F.4.i; A.497.F.4.i; A.498.F.4.i; A.499.F.4.i; A.500.F.4.i; A.501.F.4.i;
A.502.F.4.i; A.503.F.4.i; A.504.F.4.i; A.505.F.4.i; A.506.F.4.i; A.507.F.4.i;
A.508.F.4.i; A.509.F.4.i; A.510.F.4.i; A.511.F.4.i; A.512.F.4.i; A.512.F.4.i;
A.513.F.4.i; A.514.F.4.i; A.515.F.4.i; A.516.F.4.i; A.517.F.4.i; A.518.F.4.i;
A.519.F.4.i; A.520.F.4.i; A.521.F.4.i; A.522.F.4.i; A.523.F.4.i; A.524.F.4.i;
A.525.F.4.i; A.526.F.4.i; A.527.F.4.i; A.528.F.4.i; A.529.F.4.i; A.530.F.4.i;
A.531.F.4.i; A.532.F.4.i; A.533.F.4.i; A.534.F.4.i; A.535.F.4.i; A.536.F.4.i;
A.537.F.4.i; A.538.F.4.i; A.539.F.4.i; A.540.F.4.i; A.541.F.4.i; A.542.F.4.i;
A.543.F.4.i; A.544.F.4.i; A.545.F.4.i; A.546.F.4.i; A.547.F.4.i; A.548.F.4.i;
A.549.F.4.i; A.550.F.4.i; A.551.F.4.i; A.552.F.4.i; A.553.F.4.i; A.554.F.4.i;
A.555.F.4.i; A.556.F.4.i; A.557.F.4.i; A.558.F.4.i; A.559.F.4.i; A.560.F.4.i;
A.561.F.4.i; A.562.F.4.i; A.563.F.4.i; A.564.F.4.i; A.565.F.4.i; A.566.F.4.i;
A.567.F.4.i; A.568.F.4.i; A.569.F.4.i; A.570.F.4.i; A.571.F.4.i; A.572.F.4.i;
A.573.F.4.i; A.574.F.4.i; A.575.F.4.i; A.576.F.4.i; A.577.F.4.i; A.578.F.4.i;
A.579.F.4.i; A.580.F.4.i; A.581.F.4.i; A.582.F.4.i; A.583.F.4.i; A.584.F.4.i;
A.585.F.4.i; A.586.F.4.i; A.587.F.4.i; A.588.F.4.i; A.589.F.4.i; A.590.F.4.i;
A.591.F.4.i; A.592.F.4.i; A.593.F.4.i; A.594.F.4.i; A.595.F.4.i; A.596.F.4.i;
A.597.F.4.i; A.598.F.4.i; A.599.F.4.i; A.600.F.4.i; A.601.F.4.i; A.602.F.4.i;
A.603.F.4.i; A.604.F.4.i; A.605.F.4.i; A.606.F.4.i; A.607.F.4.i; A.608.F.4.i;
A.609.F.4.i; A.610.F.4.i; A.611.F.4.i; A.612.F.4.i; A.613.F.4.i; A.614.F.4.i;
A.615.F.4.i; A.616.F.4.i; A.617.F.4.i; A.618.F.4.i; A.619.F.4.i; A.620.F.4.i;
A.621.F.4.i; A.622.F.4.i; A.623.F.4.i; A.624.F.4.i; A.625.F.4.i; A.626.F.4.i;
A.627.F.4.i; A.628.F.4.i; A.629.F.4.i; A.630.F.4.i; A.631.F.4.i; A.632.F.4.i;
A.633.F.4.i; A.634.F.4.i; A.635.F.4.i; A.636.F.4.i; A.637.F.4.i; A.638.F.4.i;
A.639.F.4.i; A.640.F.4.i; A.641.F.4.i; A.642.F.4.i; A.643.F.4.i; A.644.F.4.i;
A.645.F.4.i; A.646.F.4.i; A.647.F.4.i; A.648.F.4.i; A.649.F.4.i; A.650.F.4.i;
A.651.F.4.i; A.652.F.4.i; A.653.F.4.i; A.654.F.4.i; A.655.F.4.i; A.656.F.4.i;
A.657.F.4.i; A.658.F.4.i; A.659.F.4.i; A.660.F.4.i; A.2.F.11.i; A.3.F.11.i; A.4.F.11.i;
A.5.F.11.i; A.6.F.11.i; A.7.F.11.i; A.9.F.11.i; A.10.F.11.i; A.15.F.11.i; A.100.F.11.i;
A.101.F.11.i; A.102.F.11.i; A.103.F.11.i; A.104.F.11.i; A.105.F.11.i; A.106.F.11.i;
A.107.F.11.i; A.108.F.11.i; A.109.F.11.i; A.110.F.11.i; A.111.F.11.i; A.112.F.11.i;
A.113.F.11.i; A.114.F.11.i; A.115.F.11.i; A.116.F.11.i; A.117.F.11.i; A.118.F.11.i;
A.119.F.11.i; A.120.F.11.i; A.121.F.11.i; A.122.F.11.i; A.123.F.11.i; A.124.F.11.i;
A.125.F.11.i; A.126.F.11.i; A.127.F.11.i; A.128.F.11.i; A.129.F.11.i; A.130.F.11.i;
A.131.F.11.i; A.132.F.11.i; A.133.F.11.i; A.134.F.11.i; A.135.F.11.i; A.136.F.11.i;
A.137.F.11.i; A.138.F.11.i; A.139.F.11.i; A.140.F.11.i; A.141.F.11.i; A.142.F.11.i;
A.143.F.11.i; A.144.F.11.i; A.145.F.11.i; A.146.F.11.i; A.147.F.11.i; A.148.F.11.i;
A.149.F.11.i; A.150.F.11.i; A.151.F.11.i; A.152.F.11.i; A.153.F.11.i; A.154.F.11.i;
A.155.F.11.i; A.156.F.11.i; A.157.F.11.i; A.158.F.11.i; A.159.F.11.i; A.160.F.11.i;
A.161.F.11.i; A.162.F.11.i; A.163.F.11.i; A.164.F.11.i; A.165.F.11.i; A.166.F.11.i;
A.167.F.11.i; A.168.F.11.i; A.169.F.11.i; A.170.F.11.i; A.171.F.11.i; A.172.F.11.i;
A.173.F.11.i; A.174.F.11.i; A.175.F.11.i; A.176.F.11.i; A.177.F.11.i; A.178.F.11.i;
A.179.F.11.i; A.180.F.11.i; A.181.F.11.i; A.182.F.11.i; A.183.F.11.i; A.184.F.11.i;
A.185.F.11.i; A.186.F.11.i; A.187.F.11.i; A.188.F.11.i; A.189.F.11.i; A.190.F.11.i;
A.191.F.11.i; A.192.F.11.i; A.193.F.11.i; A.194.F.11.i; A.195.F.11.i; A.196.F.11.i;
A.197.F.11.i; A.198.F.11.i; A.199.F.11.i; A.200.F.11.i; A.201.F.11.i; A.202.F.11.i;
A.203.F.11.i; A.204.F.11.i; A.205.F.11.i; A.206.F.11.i; A.207.F.11.i; A.208.F.11.i;
A.209.F.11.i; A.210.F.11.i; A.211.F.11.i; A.212.F.11.i; A.213.F.11.i; A.214.F.11.i;
A.215.F.11.i; A.216.F.11.i; A.217.F.11.i; A.218.F.11.i; A.219.F.11.i; A.220.F.11.i;
A.221.F.11.i; A.222.F.11.i; A.223.F.11.i; A.224.F.11.i; A.225.F.11.i; A.226.F.11.i;
A.227.F.11.i; A.228.F.11.i; A.229.F.11.i; A.230.F.11.i; A.231.F.11.i; A.232.F.11.i;
A.233.F.11.i; A.234.F.11.i; A.235.F.11.i; A.236.F.11.i; A.237.F.11.i; A.238.F.11.i;
A.239.F.11.i; A.240.F.11.i; A.241.F.11.i; A.242.F.11.i; A.243.F.11.i; A.244.F.11.i;
A.245.F.11.i; A.246.F.11.i; A.247.F.11.i; A.248.F.11.i; A.249.F.11.i; A.250.F.11.i;
A.251.F.11.i; A.252.F.11.i; A.253.F.11.i; A.254.F.11.i; A.255.F.11.i; A.256.F.11.i;
A.257.F.11.i; A.258.F.11.i; A.259.F.11.i; A.260.F.11.i; A.261.F.11.i; A.262.F.11.i;
A.263.F.11.i; A.264.F.11.i; A.265.F.11.i; A.266.F.11.i; A.267.F.11.i; A.268.F.11.i;
A.269.F.11.i; A.270.F.11.i; A.271.F.11.i; A.272.F.11.i; A.273.F.11.i; A.274.F.11.i;
A.275.F.11.i; A.276.F.11.i; A.277.F.11.i; A.278.F.11.i; A.279.F.11.i; A.280.F.11.i;
A.281.F.11.i; A.282.F.11.i; A.283.F.11.i; A.284.F.11.i; A.285.F.11.i; A.286.F.11.i;
A.287.F.11.i; A.288.F.11.i; A.289.F.11.i; A.290.F.11.i; A.291.F.11.i; A.292.F.11.i;
A.293.F.11.i; A.294.F.11.i; A.295.F.11.i; A.296.F.11.i; A.297.F.11.i; A.298.F.11.i;
A.299.F.11.i; A.300.F.11.i; A.301.F.11.i; A.302.F.11.i; A.303.F.11.i; A.304.F.11.i;
A.305.F.11.i; A.306.F.11.i; A.307.F.11.i; A.308.F.11.i; A.309.F.11.i; A.310.F.11.i;
A.311.F.11.i; A.312.F.11.i; A.313.F.11.i; A.314.F.11.i; A.315.F.11.i; A.316.F.11.i;
A.317.F.11.i; A.318.F.11.i; A.319.F.11.i; A.320.F.11.i; A.321.F.11.i; A.323.F.11.i;
A.324.F.11.i; A.325.F.11.i; A.326.F.11.i; A.327.F.11.i; A.328.F.11.i; A.329.F.11.i;
A.330.F.11.i; A.331.F.11.i; A.332.F.11.i; A.333.F.11.i; A.334.F.11.i; A.335.F.11.i;
A.336.F.11.i; A.337.F.11.i; A.338.F.11.i; A.339.F.11.i; A.340.F.11.i; A.341.F.11.i;
A.342.F.11.i; A.343.F.11.i; A.344.F.11.i; A.345.F.11.i; A.346.F.11.i; A.347.F.11.i;
A.348.F.11.i; A.349.F.11.i; A.350.F.11.i; A.351.F.11.i; A.352.F.11.i; A.353.F.11.i;
A.354.F.11.i; A.355.F.11.i; A.356.F.11.i; A.357.F.11.i; A.358.F.11.i; A.359.F.11.i;
A.360.F.11.i; A.361.F.11.i; A.362.F.11.i; A.363.F.11.i; A.364.F.11.i; A.365.F.11.i;
A.366.F.11.i; A.367.F.11.i; A.368.F.11.i; A.369.F.11.i; A.370.F.11.i; A.371.F.11.i;
A.372.F.11.i; A.373.F.11.i; A.374.F.11.i; A.375.F.11.i; A.376.F.11.i; A.377.F.11.i;
A.378.F.11.i; A.379.F.11.i; A.380.F.11.i; A.381.F.11.i; A.382.F.11.i; A.383.F.11.i;
A.384.F.11.i; A.385.F.11.i; A.386.F.11.i; A.387.F.11.i; A.388.F.11.i; A.389.F.11.i;
A.390.F.11.i; A.391.F.11.i; A.392.F.11.i; A.393.F.11.i; A.394.F.11.i; A.395.F.11.i;
A.396.F.11.i; A.397.F.11.i; A.398.F.11.i; A.399.F.11.i; A.400.F.11.i; A.401.F.11.i;
A.402.F.11.i; A.403.F.11.i; A.404.F.11.i; A.405.F.11.i; A.406.F.11.i; A.407.F.11.i;
A.408.F.11.i; A.409.F.11.i; A.410.F.11.i; A.411.F.11.i; A.412.F.11.i; A.413.F.11.i;
A.414.F.11.i; A.415.F.11.i; A.416.F.11.i; A.417.F.11.i; A.418.F.11.i; A.419.F.11.i;
A.420.F.11.i; A.421.F.11.i; A.422.F.11.i; A.423.F.11.i; A.424.F.11.i; A.425.F.11.i;
A.426.F.11.i; A.427.F.11.i; A.428.F.11.i; A.429.F.11.i; A.430.F.11.i; A.431.F.11.i;
A.432.F.11.i; A.433.F.11.i; A.434.F.11.i; A.435.F.11.i; A.436.F.11.i; A.437.F.11.i;
A.438.F.11.i; A.439.F.11.i; A.440.F.11.i; A.441.F.11.i; A.442.F.11.i; A.443.F.11.i;
A.444.F.11.i; A.445.F.11.i; A.446.F.11.i; A.447.F.11.i; A.448.F.11.i; A.449.F.11.i;
A.450.F.11.i; A.451.F.11.i; A.452.F.11.i; A.453.F.11.i; A.454.F.11.i; A.455.F.11.i;
A.456.F.11.i; A.457.F.11.i; A.458.F.11.i; A.459.F.11.i; A.460.F.11.i; A.461.F.11.i;
A.462.F.11.i; A.463.F.11.i; A.464.F.11.i; A.465.F.11.i; A.466.F.11.i; A.467.F.11.i;
A.468.F.11.i; A.469.F.11.i; A.470.F.11.i; A.471.F.11.i; A.472.F.11.i; A.473.F.11.i;
A.474.F.11.i; A.475.F.11.i; A.476.F.11.i; A.477.F.11.i; A.478.F.11.i; A.479.F.11.i;
A.480.F.11.i; A.481.F.11.i; A.482.F.11.i; A.483.F.11.i; A.484.F.11.i; A.485.F.11.i;
A.486.F.11.i; A.487.F.11.i; A.488.F.11.i; A.489.F.11.i; A.490.F.11.i; A.491.F.11.i;
A.492.F.11.i; A.493.F.11.i; A.494.F.11.i; A.495.F.11.i; A.496.F.11.i; A.497.F.11.i;
A.498.F.11.i; A.499.F.11.i; A.500.F.11.i; A.501.F.11.i; A.502.F.11.i; A.503.F.11.i;
A.504.F.11.i; A.505.F.11.i; A.506.F.11.i; A.507.F.11.i; A.508.F.11.i; A.509.F.11.i;
A.510.F.11.i; A.511.F.11.i; A.512.F.11.i; A.512.F.11.i; A.513.F.11.i; A.514.F.11.i;
A.515.F.11.i; A.516.F.11.i; A.517.F.11.i; A.518.F.11.i; A.519.F.11.i; A.520.F.11.i;
A.521.F.11.i; A.522.F.11.i; A.523.F.11.i; A.524.F.11.i; A.525.F.11.i; A.526.F.11.i;
A.527.F.11.i; A.528.F.11.i; A.529.F.11.i; A.530.F.11.i; A.531.F.11.i; A.532.F.11.i;
A.533.F.11.i; A.534.F.11.i; A.535.F.11.i; A.536.F.11.i; A.537.F.11.i; A.538.F.11.i;
A.539.F.11.i; A.540.F.11.i; A.541.F.11.i; A.542.F.11.i; A.543.F.11.i; A.544.F.11.i;
A.545.F.11.i; A.546.F.11.i; A.547.F.11.i; A.548.F.11.i; A.549.F.11.i; A.550.F.11.i;
A.551.F.11.i; A.552.F.11.i; A.553.F.11.i; A.554.F.11.i; A.555.F.11.i; A.556.F.11.i;
A.557.F.11.i; A.558.F.11.i; A.559.F.11.i; A.560.F.11.i; A.561.F.11.i; A.562.F.11.i;
A.563.F.11.i; A.564.F.11.i; A.565.F.11.i; A.566.F.11.i; A.567.F.11.i; A.568.F.11.i;
A.569.F.11.i; A.570.F.11.i; A.571.F.11.i; A.572.F.11.i; A.573.F.11.i; A.574.F.11.i;
A.575.F.11.i; A.576.F.11.i; A.577.F.11.i; A.578.F.11.i; A.579.F.11.i; A.580.F.11.i;
A.581.F.11.i; A.582.F.11.i; A.583.F.11.i; A.584.F.11.i; A.585.F.11.i; A.586.F.11.i;
A.587.F.11.i; A.588.F.11.i; A.589.F.11.i; A.590.F.11.i; A.591.F.11.i; A.592.F.11.i;
A.593.F.11.i; A.594.F.11.i; A.595.F.11.i; A.596.F.11.i; A.597.F.11.i; A.598.F.11.i;
A.599.F.11.i; A.600.F.11.i; A.601.F.11.i; A.602.F.11.i; A.603.F.11.i; A.604.F.11.i;
A.605.F.11.i; A.606.F.11.i; A.607.F.11.i; A.608.F.11.i; A.609.F.11.i; A.610.F.11.i;
A.611.F.11.i; A.612.F.11.i; A.613.F.11.i; A.614.F.11.i; A.615.F.11.i; A.616.F.11.i;
A.617.F.11.i; A.618.F.11.i; A.619.F.11.i; A.620.F.11.i; A.621.F.11.i; A.622.F.11.i;
A.623.F.11.i; A.624.F.11.i; A.625.F.11.i; A.626.F.11.i; A.627.F.11.i; A.628.F.11.i;
A.629.F.11.i; A.630.F.11.i; A.631.F.11.i; A.632.F.11.i; A.633.F.11.i; A.634.F.11.i;
A.635.F.11.i; A.636.F.11.i; A.637.F.11.i; A.638.F.11.i; A.639.F.11.i; A.640.F.11.i;
A.641.F.11.i; A.642.F.11.i; A.643.F.11.i; A.644.F.11.i; A.645.F.11.i; A.646.F.11.i;
A.647.F.11.i; A.648.F.11.i; A.649.F.11.i; A.650.F.11.i; A.651.F.11.i; A.652.F.11.i;
A.653.F.11.i; A.654.F.11.i; A.655.F.11.i; A.656.F.11.i; A.657.F.11.i; A.658.F.11.i;
A.659.F.11.i; A.660.F.11.i; A.2.a.44.i; A.3.a.44.i; A.4.a.44.i; A.5.a.44.i; A.9.a.44.i;
A.100.a.44.i; A.101.a.44.i; A.102.a.44.i; A.103.a.44.i; A.104.a.44.i; A.105.a.44.i;
A.106.a.44.i; A.107.a.44.i; A.108.a.44.i; A.109.a.44.i; A.110.a.44.i; A.111.a.44.i;
A.112.a.44.i; A.113.a.44.i; A.114.a.44.i; A.115.a.44.i; A.116.a.44.i; A.117.a.44.i;
A.118.a.44.i; A.119.a.44.i; A.120.a.44.i; A.121.a.44.i; A.122.a.44.i; A.123.a.44.i;
A.124.a.44.i; A.125.a.44.i; A.126.a.44.i; A.127.a.44.i; A.128.a.44.i; A.129.a.44.i;
A.130.a.44.i; A.131.a.44.i; A.132.a.44.i; A.133.a.44.i; A.134.a.44.i; A.135.a.44.i;
A.136.a.44.i; A.137.a.44.i; A.138.a.44.i; A.139.a.44.i; A.140.a.44.i; A.141.a.44.i;
A.142.a.44.i; A.143.a.44.i; A.144.a.44.i; A.145.a.44.i; A.146.a.44.i; A.147.a.44.i;
A.148.a.44.i; A.149.a.44.i; A.150.a.44.i; A.151.a.44.i; A.152.a.44.i; A.153.a.44.i;
A.154.a.44.i; A.155.a.44.i; A.156.a.44.i; A.157.a.44.i; A.158.a.44.i; A.159.a.44.i;
A.160.a.44.i; A.161.a.44.i; A.162.a.44.i; A.163.a.44.i; A.164.a.44.i; A.165.a.44.i;
A.166.a.44.i; A.167.a.44.i; A.168.a.44.i; A.169.a.44.i; A.170.a.44.i; A.171.a.44.i;
A.172.a.44.i; A.173.a.44.i; A.174.a.44.i; A.175.a.44.i; A.176.a.44.i; A.177.a.44.i;
A.178.a.44.i; A.179.a.44.i; A.180.a.44.i; A.181.a.44.i; A.182.a.44.i; A.183.a.44.i;
A.184.a.44.i; A.185.a.44.i; A.186.a.44.i; A.187.a.44.i; A.188.a.44.i; A.189.a.44.i;
A.190.a.44.i; A.191.a.44.i; A.192.a.44.i; A.193.a.44.i; A.194.a.44.i; A.195.a.44.i;
A.196.a.44.i; A.197.a.44.i; A.198.a.44.i; A.199.a.44.i; A.200.a.44.i; A.201.a.44.i;
A.202.a.44.i; A.203.a.44.i; A.204.a.44.i; A.205.a.44.i; A.206.a.44.i; A.207.a.44.i;
A.208.a.44.i; A.209.a.44.i; A.210.a.44.i; A.211.a.44.i; A.212.a.44.i; A.213.a.44.i;
A.214.a.44.i; A.215.a.44.i; A.216.a.44.i; A.217.a.44.i; A.218.a.44.i; A.219.a.44.i;
A.220.a.44.i; A.221.a.44.i; A.222.a.44.i; A.223.a.44.i; A.224.a.44.i; A.225.a.44.i;
A.226.a.44.i; A.227.a.44.i; A.228.a.44.i; A.229.a.44.i; A.230.a.44.i; A.231.a.44.i;
A.232.a.44.i; A.233.a.44.i; A.234.a.44.i; A.235.a.44.i; A.236.a.44.i; A.237.a.44.i;
A.238.a.44.i; A.239.a.44.i; A.240.a.44.i; A.241.a.44.i; A.242.a.44.i; A.243.a.44.i;
A.244.a.44.i; A.245.a.44.i; A.246.a.44.i; A.247.a.44.i; A.248.a.44.i; A.249.a.44.i;
A.250.a.44.i; A.251.a.44.i; A.252.a.44.i; A.253.a.44.i; A.254.a.44.i; A.255.a.44.i;
A.256.a.44.i; A.257.a.44.i; A.258.a.44.i; A.259.a.44.i; A.260.a.44.i; A.261.a.44.i;
A.262.a.44.i; A.263.a.44.i; A.264.a.44.i; A.265.a.44.i; A.266.a.44.i; A.267.a.44.i;
A.268.a.44.i; A.269.a.44.i; A.270.a.44.i; A.271.a.44.i; A.272.a.44.i; A.273.a.44.i;
A.274.a.44.i; A.275.a.44.i; A.276.a.44.i; A.277.a.44.i; A.278.a.44.i; A.279.a.44.i;
A.280.a.44.i; A.281.a.44.i; A.282.a.44.i; A.283.a.44.i; A.284.a.44.i; A.285.a.44.i;
A.286.a.44.i; A.287.a.44.i; A.288.a.44.i; A.289.a.44.i; A.290.a.44.i; A.291.a.44.i;
A.292.a.44.i; A.293.a.44.i; A.294.a.44.i; A.295.a.44.i; A.296.a.44.i; A.297.a.44.i;
A.298.a.44.i; A.299.a.44.i; A.300.a.44.i; A.301.a.44.i; A.302.a.44.i; A.303.a.44.i;
A.304.a.44.i; A.305.a.44.i; A.306.a.44.i; A.307.a.44.i; A.308.a.44.i; A.309.a.44.i;
A.310.a.44.i; A.311.a.44.i; A.312.a.44.i; A.313.a.44.i; A.314.a.44.i; A.315.a.44.i;
A.316.a.44.i; A.317.a.44.i; A.318.a.44.i; A.319.a.44.i; A.320.a.44.i; A.321.a.44.i;
A.322.a.44.i; A.323.a.44.i; A.324.a.44.i; A.325.a.44.i; A.326.a.44.i; A.327.a.44.i;
A.328.a.44.i; A.329.a.44.i; A.330.a.44.i; A.331.a.44.i; A.332.a.44.i; A.333.a.44.i;
A.334.a.44.i; A.335.a.44.i; A.336.a.44.i; A.337.a.44.i; A.338.a.44.i; A.339.a.44.i;
A.340.a.44.i; A.341.a.44.i; A.342.a.44.i; A.343.a.44.i; A.344.a.44.i; A.345.a.44.i;
A.346.a.44.i; A.347.a.44.i; A.348.a.44.i; A.349.a.44.i; A.350.a.44.i; A.351.a.44.i;
A.352.a.44.i; A.353.a.44.i; A.354.a.44.i; A.355.a.44.i; A.356.a.44.i; A.357.a.44.i;
A.358.a.44.i; B.2.a.44.i; B.3.a.44.i; B.4.a.44.i; B.5.a.44.i; B.9.a.44.i; B.100.a.44.i;
B.101.a.44.i; B.102.a.44.i; B.103.a.44.i; B.104.a.44.i; B.105.a.44.i; B.106.a.44.i;
B.107.a.44.i; B.108.a.44.i; B.109.a.44.i; B.110.a.44.i; B.111.a.44.i; B.112.a.44.i;
B.113.a.44.i; B.114.a.44.i; B.115.a.44.i; B.116.a.44.i; B.117.a.44.i; B.118.a.44.i;
B.119.a.44.i; B.120.a.44.i; B.121.a.44.i; B.122.a.44.i; B.123.a.44.i; B.124.a.44.i;
B.125.a.44.i; B.126.a.44.i; B.127.a.44.i; B.128.a.44.i; B.129.a.44.i; B.130.a.44.i;
B.131.a.44.i; B.132.a.44.i; B.133.a.44.i; B.134.a.44.i; B.135.a.44.i; B.136.a.44.i;
B.137.a.44.i; B.138.a.44.i; B.139.a.44.i; B.140.a.44.i; B.141.a.44.i; B.142.a.44.i;
B.143.a.44.i; B.144.a.44.i; B.145.a.44.i; B.146.a.44.i; B.147.a.44.i; B.148.a.44.i;
B.149.a.44.i; B.150.a.44.i; B.151.a.44.i; B.152.a.44.i; B.153.a.44.i; B.154.a.44.i;
B.155.a.44.i; B.156.a.44.i; B.157.a.44.i; B.158.a.44.i; B.159.a.44.i; B.160.a.44.i;
B.161.a.44.i; B.162.a.44.i; B.163.a.44.i; B.164.a.44.i; B.165.a.44.i; B.166.a.44.i;
B.167.a.44.i; B.168.a.44.i; B.169.a.44.i; B.170.a.44.i; B.171.a.44.i; B.172.a.44.i;
B.173.a.44.i; B.174.a.44.i; B.175.a.44.i; B.176.a.44.i; B.177.a.44.i; B.178.a.44.i;
B.179.a.44.i; B.180.a.44.i; B.181.a.44.i; B.182.a.44.i; B.183.a.44.i; B.184.a.44.i;
B.185.a.44.i; B.186.a.44.i; B.187.a.44.i; B.188.a.44.i; B.189.a.44.i; B.190.a.44.i;
B.191.a.44.i; B.192.a.44.i; B.193.a.44.i; B.194.a.44.i; B.195.a.44.i; B.196.a.44.i;
B.197.a.44.i; B.198.a.44.i; B.199.a.44.i; B.200.a.44.i; B.201.a.44.i; B.202.a.44.i;
B.203.a.44.i; B.204.a.44.i; B.205.a.44.i; B.206.a.44.i; B.207.a.44.i; B.208.a.44.i;
B.209.a.44.i; B.210.a.44.i; B.211.a.44.i; B.212.a.44.i; B.213.a.44.i; B.214.a.44.i;
B.215.a.44.i; B.216.a.44.i; B.217.a.44.i; B.218.a.44.i; B.219.a.44.i; B.220.a.44.i;
B.221.a.44.i; B.222.a.44.i; B.223.a.44.i; B.224.a.44.i; B.225.a.44.i; B.226.a.44.i;
B.227.a.44.i; B.228.a.44.i; B.229.a.44.i; B.230.a.44.i; B.231.a.44.i; B.232.a.44.i;
B.233.a.44.i; B.234.a.44.i; B.235.a.44.i; B.236.a.44.i; B.237.a.44.i; B.238.a.44.i;
B.239.a.44.i; B.240.a.44.i; B.241.a.44.i; B.242.a.44.i; B.243.a.44.i; B.244.a.44.i;
B.245.a.44.i; B.246.a.44.i; B.247.a.44.i; B.248.a.44.i; B.249.a.44.i; B.250.a.44.i;
B.251.a.44.i; B.252.a.44.i; B.253.a.44.i; B.254.a.44.i; B.255.a.44.i; B.256.a.44.i;
B.257.a.44.i; B.258.a.44.i; B.259.a.44.i; B.260.a.44.i; B.261.a.44.i; B.262.a.44.i;
B.263.a.44.i; B.264.a.44.i; B.265.a.44.i; B.266.a.44.i; B.267.a.44.i; B.268.a.44.i;
B.269.a.44.i; B.270.a.44.i; B.271.a.44.i; B.272.a.44.i; B.273.a.44.i; B.274.a.44.i;
B.275.a.44.i; B.276.a.44.i; B.277.a.44.i; B.278.a.44.i; B.279.a.44.i; B.280.a.44.i;
B.281.a.44.i; B.282.a.44.i; B.283.a.44.i; B.284.a.44.i; B.285.a.44.i; B.286.a.44.i;
B.287.a.44.i; B.288.a.44.i; B.289.a.44.i; B.290.a.44.i; B.291.a.44.i; B.292.a.44.i;
B.293.a.44.i; B.294.a.44.i; B.295.a.44.i; B.296.a.44.i; B.297.a.44.i; B.298.a.44.i;
B.299.a.44.i; B.300.a.44.i; B.301.a.44.i; B.302.a.44.i; B.303.a.44.i; B.304.a.44.i;
B.305.a.44.i; B.306.a.44.i; B.307.a.44.i; B.308.a.44.i; B.309.a.44.i; B.310.a.44.i;
B.311.a.44.i; B.312.a.44.i; B.313.a.44.i; B.314.a.44.i; B.315.a.44.i; B.316.a.44.i;
B.317.a.44.i; B.318.a.44.i; B.319.a.44.i; B.320.a.44.i; B.321.a.44.i; B.322.a.44.i;
B.323.a.44.i; B.324.a.44.i; B.325.a.44.i; B.326.a.44.i; B.327.a.44.i; B.328.a.44.i;
B.329.a.44.i; B.330.a.44.i; B.331.a.44.i; B.332.a.44.i; B.333.a.44.i; B.334.a.44.i;
B.335.a.44.i; B.336.a.44.i; B.337.a.44.i; B.338.a.44.i; B.339.a.44.i; B.340.a.44.i;
B.341.a.44.i; B.342.a.44.i; B.343.a.44.i; B.344.a.44.i; B.345.a.44.i; B.346.a.44.i;
B.347.a.44.i; B.348.a.44.i; B.349.a.44.i; B.350.a.44.i; B.351.a.44.i; B.352.a.44.i;
B.353.a.44.i; B.354.a.44.i; B.355.a.44.i; B.356.a.44.i; B.357.a.44.i; B.358.a.44.i;
E.2.a.44.i; E.3.a.44.i; E.4.a.44.i; E.5.a.44.i; E.9.a.44.i; E.100.a.44.i; E.101.a.44.i;
E.102.a.44.i; E.103.a.44.i; E.104.a.44.i; E.105.a.44.i; E.106.a.44.i; E.107.a.44.i;
E.108.a.44.i; E.109.a.44.i; E.110.a.44.i; E.111.a.44.i; E.112.a.44.i; E.113.a.44.i;
E.114.a.44.i; E.115.a.44.i; E.116.a.44.i; E.117.a.44.i; E.118.a.44.i; E.119.a.44.i;
E.120.a.44.i; E.121.a.44.i; E.122.a.44.i; E.123.a.44.i; E.124.a.44.i; E.125.a.44.i;
E.126.a.44.i; E.127.a.44.i; E.128.a.44.i; E.129.a.44.i; E.130.a.44.i; E.131.a.44.i;
E.132.a.44.i; E.133.a.44.i; E.134.a.44.i; E.135.a.44.i; E.136.a.44.i; E.137.a.44.i;
E.138.a.44.i; E.139.a.44.i; E.140.a.44.i; E.141.a.44.i; E.142.a.44.i; E.143.a.44.i;
E.144.a.44.i; E.145.a.44.i; E.146.a.44.i; E.147.a.44.i; E.148.a.44.i; E.149.a.44.i;
E.150.a.44.i; E.151.a.44.i; E.152.a.44.i; E.153.a.44.i; E.154.a.44.i; E.155.a.44.i;
E.156.a.44.i; E.157.a.44.i; E.158.a.44.i; E.159.a.44.i; E.160.a.44.i; E.161.a.44.i;
E.162.a.44.i; E.163.a.44.i; E.164.a.44.i; E.165.a.44.i; E.166.a.44.i; E.167.a.44.i;
E.168.a.44.i; E.169.a.44.i; E.170.a.44.i; E.171.a.44.i; E.172.a.44.i; E.173.a.44.i;
E.174.a.44.i; E.175.a.44.i; E.176.a.44.i; E.177.a.44.i; E.178.a.44.i; E.179.a.44.i;
E.180.a.44.i; E.181.a.44.i; E.182.a.44.i; E.183.a.44.i; E.184.a.44.i; E.185.a.44.i;
E.186.a.44.i; E.187.a.44.i; E.188.a.44.i; E.189.a.44.i; E.190.a.44.i; E.191.a.44.i;
E.192.a.44.i; E.193.a.44.i; E.194.a.44.i; E.195.a.44.i; E.196.a.44.i; E.197.a.44.i;
E.198.a.44.i; E.199.a.44.i; E.200.a.44.i; E.201.a.44.i; E.202.a.44.i; E.203.a.44.i;
E.204.a.44.i; E.205.a.44.i; E.206.a.44.i; E.207.a.44.i; E.208.a.44.i; E.209.a.44.i;
E.210.a.44.i; E.211.a.44.i; E.212.a.44.i; E.213.a.44.i; E.214.a.44.i; E.215.a.44.i;
E.216.a.44.i; E.217.a.44.i; E.218.a.44.i; E.219.a.44.i; E.220.a.44.i; E.221.a.44.i;
E.222.a.44.i; E.223.a.44.i; E.224.a.44.i; E.225.a.44.i; E.226.a.44.i; E.227.a.44.i;
E.228.a.44.i; E.229.a.44.i; E.230.a.44.i; E.231.a.44.i; E.232.a.44.i; E.233.a.44.i;
E.234.a.44.i; E.235.a.44.i; E.236.a.44.i; E.237.a.44.i; E.238.a.44.i; E.239.a.44.i;
E.240.a.44.i; E.241.a.44.i; E.242.a.44.i; E.243.a.44.i; E.244.a.44.i; E.245.a.44.i;
E.246.a.44.i; E.247.a.44.i; E.248.a.44.i; E.249.a.44.i; E.250.a.44.i; E.251.a.44.i;
E.252.a.44.i; E.253.a.44.i; E.254.a.44.i; E.255.a.44.i; E.256.a.44.i; E.257.a.44.i;
E.258.a.44.i; E.259.a.44.i; E.260.a.44.i; E.261.a.44.i; E.262.a.44.i; E.263.a.44.i;
E.264.a.44.i; E.265.a.44.i; E.266.a.44.i; E.267.a.44.i; E.268.a.44.i; E.269.a.44.i;
E.270.a.44.i; E.271.a.44.i; E.272.a.44.i; E.273.a.44.i; E.274.a.44.i; E.275.a.44.i;
E.276.a.44.i; E.277.a.44.i; E.278.a.44.i; E.279.a.44.i; E.280.a.44.i; E.281.a.44.i;
E.282.a.44.i; E.283.a.44.i; E.284.a.44.i; E.285.a.44.i; E.286.a.44.i; E.287.a.44.i;
E.288.a.44.i; E.289.a.44.i; E.290.a.44.i; E.291.a.44.i; E.292.a.44.i; E.293.a.44.i;
E.294.a.44.i; E.295.a.44.i; E.296.a.44.i; E.297.a.44.i; E.298.a.44.i; E.299.a.44.i;
E.300.a.44.i; E.301.a.44.i; E.302.a.44.i; E.303.a.44.i; E.304.a.44.i; E.305.a.44.i;
E.306.a.44.i; E.307.a.44.i; E.308.a.44.i; E.309.a.44.i; E.310.a.44.i; E.311.a.44.i;
E.312.a.44.i; E.313.a.44.i; E.314.a.44.i; E.315.a.44.i; E.316.a.44.i; E.317.a.44.i;
E.318.a.44.i; E.319.a.44.i; E.320.a.44.i; E.321.a.44.i; E.322.a.44.i; E.323.a.44.i;
E.324.a.44.i; E.325.a.44.i; E.326.a.44.i; E.327.a.44.i; E.328.a.44.i; E.329.a.44.i;
E.330.a.44.i; E.331.a.44.i; E.332.a.44.i; E.333.a.44.i; E.334.a.44.i; E.335.a.44.i;
E.336.a.44.i; E.337.a.44.i; E.338.a.44.i; E.339.a.44.i; E.340.a.44.i; E.341.a.44.i;
E.342.a.44.i; E.343.a.44.i; E.344.a.44.i; E.345.a.44.i; E.346.a.44.i; E.347.a.44.i;
E.348.a.44.i; E.349.a.44.i; E.350.a.44.i; E.351.a.44.i; E.352.a.44.i; E.353.a.44.i;
E.354.a.44.i; E.355.a.44.i; E.356.a.44.i; E.357.a.44.i; E.358.a.44.i; B.2.a.4.i; B.3.a.4.i;
B.4.a.4.i; B.5.a.4.i; B.9.a.4.i; B.100.a.4.i; B.101.a.4.i; B.102.a.4.i; B.103.a.4.i;
B.104.a.4.i; B.105.a.4.i; B.106.a.4.i; B.107.a.4.i; B.108.a.4.i; B.109.a.4.i; B.110.a.4.i;
B.111.a.4.i; B.112.a.4.i; B.113.a.4.i; B.114.a.4.i; B.115.a.4.i; B.116.a.4.i; B.117.a.4.i;
B.118.a.4.i; B.119.a.4.i; B.120.a.4.i; B.121.a.4.i; B.122.a.4.i; B.123.a.4.i; B.124.a.4.i;
B.125.a.4.i; B.126.a.4.i; B.127.a.4.i; B.128.a.4.i; B.129.a.4.i; B.130.a.4.i; B.131.a.4.i;
B.132.a.4.i; B.133.a.4.i; B.134.a.4.i; B.135.a.4.i; B.136.a.4.i; B.137.a.4.i; B.138.a.4.i;
B.139.a.4.i; B.140.a.4.i; B.141.a.4.i; B.142.a.4.i; B.143.a.4.i; B.144.a.4.i; B.145.a.4.i;
B.146.a.4.i; B.147.a.4.i; B.148.a.4.i; B.149.a.4.i; B.150.a.4.i; B.151.a.4.i; B.152.a.4.i;
B.153.a.4.i; B.154.a.4.i; B.155.a.4.i; B.156.a.4.i; B.157.a.4.i; B.158.a.4.i; B.159.a.4.i;
B.160.a.4.i; B.161.a.4.i; B.162.a.4.i; B.163.a.4.i; B.164.a.4.i; B.165.a.4.i; B.166.a.4.i;
B.167.a.4.i; B.168.a.4.i; B.169.a.4.i; B.170.a.4.i; B.171.a.4.i; B.172.a.4.i; B.173.a.4.i;
B.174.a.4.i; B.175.a.4.i; B.176.a.4.i; B.177.a.4.i; B.178.a.4.i; B.179.a.4.i; B.180.a.4.i;
B.181.a.4.i; B.182.a.4.i; B.183.a.4.i; B.184.a.4.i; B.185.a.4.i; B.186.a.4.i; B.187.a.4.i;
B.188.a.4.i; B.189.a.4.i; B.190.a.4.i; B.191.a.4.i; B.192.a.4.i; B.193.a.4.i; B.194.a.4.i;
B.195.a.4.i; B.196.a.4.i; B.197.a.4.i; B.198.a.4.i; B.199.a.4.i; B.200.a.4.i; B.201.a.4.i;
B.202.a.4.i; B.203.a.4.i; B.204.a.4.i; B.205.a.4.i; B.206.a.4.i; B.207.a.4.i; B.208.a.4.i;
B.209.a.4.i; B.210.a.4.i; B.211.a.4.i; B.212.a.4.i; B.213.a.4.i; B.214.a.4.i; B.215.a.4.i;
B.216.a.4.i; B.217.a.4.i; B.218.a.4.i; B.219.a.4.i; B.220.a.4.i; B.221.a.4.i; B.222.a.4.i;
B.223.a.4.i; B.224.a.4.i; B.225.a.4.i; B.226.a.4.i; B.227.a.4.i; B.228.a.4.i; B.229.a.4.i;
B.230.a.4.i; B.231.a.4.i; B.232.a.4.i; B.233.a.4.i; B.234.a.4.i; B.235.a.4.i; B.236.a.4.i;
B.237.a.4.i; B.238.a.4.i; B.239.a.4.i; B.240.a.4.i; B.241.a.4.i; B.242.a.4.i; B.243.a.4.i;
B.244.a.4.i; B.245.a.4.i; B.246.a.4.i; B.247.a.4.i; B.248.a.4.i; B.249.a.4.i; B.250.a.4.i;
B.251.a.4.i; B.252.a.4.i; B.253.a.4.i; B.254.a.4.i; B.255.a.4.i; B.256.a.4.i; B.257.a.4.i;
B.258.a.4.i; B.259.a.4.i; B.260.a.4.i; B.261.a.4.i; B.262.a.4.i; B.263.a.4.i; B.264.a.4.i;
B.265.a.4.i; B.266.a.4.i; B.267.a.4.i; B.268.a.4.i; B.269.a.4.i; B.270.a.4.i; B.271.a.4.i;
B.272.a.4.i; B.273.a.4.i; B.274.a.4.i; B.275.a.4.i; B.276.a.4.i; B.277.a.4.i; B.278.a.4.i;
B.279.a.4.i; B.280.a.4.i; B.281.a.4.i; B.282.a.4.i; B.283.a.4.i; B.284.a.4.i; B.285.a.4.i;
B.286.a.4.i; B.287.a.4.i; B.288.a.4.i; B.289.a.4.i; B.290.a.4.i; B.291.a.4.i; B.292.a.4.i;
B.293.a.4.i; B.294.a.4.i; B.295.a.4.i; B.296.a.4.i; B.297.a.4.i; B.298.a.4.i; B.299.a.4.i;
B.300.a.4.i; B.301.a.4.i; B.302.a.4.i; B.303.a.4.i; B.304.a.4.i; B.305.a.4.i; B.306.a.4.i;
B.307.a.4.i; B.308.a.4.i; B.309.a.4.i; B.310.a.4.i; B.311.a.4.i; B.312.a.4.i; B.313.a.4.i;
B.314.a.4.i; B.315.a.4.i; B.316.a.4.i; B.317.a.4.i; B.318.a.4.i; B.319.a.4.i; B.320.a.4.i;
B.321.a.4.i; B.322.a.4.i; B.323.a.4.i; B.324.a.4.i; B.325.a.4.i; B.326.a.4.i; B.327.a.4.i;
B.328.a.4.i; B.329.a.4.i; B.330.a.4.i; B.331.a.4.i; B.332.a.4.i; B.333.a.4.i; B.334.a.4.i;
B.335.a.4.i; B.336.a.4.i; B.337.a.4.i; B.338.a.4.i; B.339.a.4.i; B.340.a.4.i; B.341.a.4.i;
B.342.a.4.i; B.343.a.4.i; B.344.a.4.i; B.345.a.4.i; B.346.a.4.i; B.347.a.4.i; B.348.a.4.i;
B.349.a.4.i; B.350.a.4.i; B.351.a.4.i; B.352.a.4.i; B.353.a.4.i; B.354.a.4.i; B.355.a.4.i;
B.356.a.4.i; B.357.a.4.i; B.358.a.4.i; E.2.a.4.i; E.3.a.4.i; E.4.a.4.i; E.5.a.4.i; E.9.a.4.i;
E.100.a.4.i; E.101.a.4.i; E.102.a.4.i; E.103.a.4.i; E.104.a.4.i; E.105.a.4.i; E.106.a.4.i;
E.107.a.4.i; E.108.a.4.i; E.109.a.4.i; E.110.a.4.i; E.111.a.4.i; E.112.a.4.i; E.113.a.4.i;
E.114.a.4.i; E.115.a.4.i; E.116.a.4.i; E.117.a.4.i; E.118.a.4.i; E.119.a.4.i; E.120.a.4.i;
E.121.a.4.i; E.122.a.4.i; E.123.a.4.i; E.124.a.4.i; E.125.a.4.i; E.126.a.4.i; E.127.a.4.i;
E.128.a.4.i; E.129.a.4.i; E.130.a.4.i; E.131.a.4.i; E.132.a.4.i; E.133.a.4.i; E.134.a.4.i;
E.135.a.4.i; E.136.a.4.i; E.137.a.4.i; E.138.a.4.i; E.139.a.4.i; E.140.a.4.i; E.141.a.4.i;
E.142.a.4.i; E.143.a.4.i; E.144.a.4.i; E.145.a.4.i; E.146.a.4.i; E.147.a.4.i; E.148.a.4.i;
E.149.a.4.i; E.150.a.4.i; E.151.a.4.i; E.152.a.4.i; E.153.a.4.i; E.154.a.4.i; E.155.a.4.i;
E.156.a.4.i; E.157.a.4.i; E.158.a.4.i; E.159.a.4.i; E.160.a.4.i; E.161.a.4.i; E.162.a.4.i;
E.163.a.4.i; E.164.a.4.i; E.165.a.4.i; E.166.a.4.i; E.167.a.4.i; E.168.a.4.i; E.169.a.4.i;
E.170.a.4.i; E.171.a.4.i; E.172.a.4.i; E.173.a.4.i; E.174.a.4.i; E.175.a.4.i; E.176.a.4.i;
E.177.a.4.i; E.178.a.4.i; E.179.a.4.i; E.180.a.4.i; E.181.a.4.i; E.182.a.4.i; E.183.a.4.i;
E.184.a.4.i; E.185.a.4.i; E.186.a.4.i; E.187.a.4.i; E.188.a.4.i; E.189.a.4.i; E.190.a.4.i;
E.191.a.4.i; E.192.a.4.i; E.193.a.4.i; E.194.a.4.i; E.195.a.4.i; E.196.a.4.i; E.197.a.4.i;
E.198.a.4.i; E.199.a.4.i; E.200.a.4.i; E.201.a.4.i; E.202.a.4.i; E.203.a.4.i; E.204.a.4.i;
E.205.a.4.i; E.206.a.4.i; E.207.a.4.i; E.208.a.4.i; E.209.a.4.i; E.210.a.4.i; E.211.a.4.i;
E.212.a.4.i; E.213.a.4.i; E.214.a.4.i; E.215.a.4.i; E.216.a.4.i; E.217.a.4.i; E.218.a.4.i;
E.219.a.4.i; E.220.a.4.i; E.221.a.4.i; E.222.a.4.i; E.223.a.4.i; E.224.a.4.i; E.225.a.4.i;
E.226.a.4.i; E.227.a.4.i; E.228.a.4.i; E.229.a.4.i; E.230.a.4.i; E.231.a.4.i; E.232.a.4.i;
E.233.a.4.i; E.234.a.4.i; E.235.a.4.i; E.236.a.4.i; E.237.a.4.i; E.238.a.4.i; E.239.a.4.i;
E.240.a.4.i; E.241.a.4.i; E.242.a.4.i; E.243.a.4.i; E.244.a.4.i; E.245.a.4.i; E.246.a.4.i;
E.247.a.4.i; E.248.a.4.i; E.249.a.4.i; E.250.a.4.i; E.251.a.4.i; E.252.a.4.i; E.253.a.4.i;
E.254.a.4.i; E.255.a.4.i; E.256.a.4.i; E.257.a.4.i; E.258.a.4.i; E.259.a.4.i; E.260.a.4.i;
E.261.a.4.i; E.262.a.4.i; E.263.a.4.i; E.264.a.4.i; E.265.a.4.i; E.266.a.4.i; E.267.a.4.i;
E.268.a.4.i; E.269.a.4.i; E.270.a.4.i; E.271.a.4.i; E.272.a.4.i; E.273.a.4.i; E.274.a.4.i;
E.275.a.4.i; E.276.a.4.i; E.277.a.4.i; E.278.a.4.i; E.279.a.4.i; E.280.a.4.i; E.281.a.4.i;
E.282.a.4.i; E.283.a.4.i; E.284.a.4.i; E.285.a.4.i; E.286.a.4.i; E.287.a.4.i; E.288.a.4.i;
E.289.a.4.i; E.290.a.4.i; E.291.a.4.i; E.292.a.4.i; E.293.a.4.i; E.294.a.4.i; E.295.a.4.i;
E.296.a.4.i; E.297.a.4.i; E.298.a.4.i; E.299.a.4.i; E.300.a.4.i; E.301.a.4.i; E.302.a.4.i;
E.303.a.4.i; E.304.a.4.i; E.305.a.4.i; E.306.a.4.i; E.307.a.4.i; E.308.a.4.i; E.309.a.4.i;
E.310.a.4.i; E.311.a.4.i; E.312.a.4.i; E.313.a.4.i; E.314.a.4.i; E.315.a.4.i; E.316.a.4.i;
E.317.a.4.i; E.318.a.4.i; E.319.a.4.i; E.320.a.4.i; E.321.a.4.i; E.322.a.4.i; E.323.a.4.i;
E.324.a.4.i; E.325.a.4.i; E.326.a.4.i; E.327.a.4.i; E.328.a.4.i; E.329.a.4.i; E.330.a.4.i;
E.331.a.4.i; E.332.a.4.i; E.333.a.4.i; E.334.a.4.i; E.335.a.4.i; E.336.a.4.i; E.337.a.4.i;
E.338.a.4.i; E.339.a.4.i; E.340.a.4.i; E.341.a.4.i; E.342.a.4.i; E.343.a.4.i; E.344.a.4.i;
E.345.a.4.i; E.346.a.4.i; E.347.a.4.i; E.348.a.4.i; E.349.a.4.i; E.350.a.4.i; E.351.a.4.i;
E.352.a.4.i; E.353.a.4.i; E.354.a.4.i; E.355.a.4.i; E.356.a.4.i; E.357.a.4.i; E.358.a.4.i;
B.2.a.11.i; B.3.a.11.i; B.4.a.11.i; B.5.a.11.i; B.9.a.11.i; B.100.a.11.i; B.101.a.11.i;
B.102.a.11.i; B.103.a.11.i; B.104.a.11.i; B.105.a.11.i; B.106.a.11.i; B.107.a.11.i;
B.108.a.11.i; B.109.a.11.i; B.110.a.11.i; B.111.a.11.i; B.112.a.11.i; B.113.a.11.i;
B.114.a.11.i; B.115.a.11.i; B.116.a.11.i; B.117.a.11.i; B.118.a.11.i; B.119.a.11.i;
B.120.a.11.i; B.121.a.11.i; B.122.a.11.i; B.123.a.11.i; B.124.a.11.i; B.125.a.11.i;
B.126.a.11.i; B.127.a.11.i; B.128.a.11.i; B.129.a.11.i; B.130.a.11.i; B.131.a.11.i;
B.132.a.11.i; B.133.a.11.i; B.134.a.11.i; B.135.a.11.i; B.136.a.11.i; B.137.a.11.i;
B.138.a.11.i; B.139.a.11.i; B.140.a.11.i; B.141.a.11.i; B.142.a.11.i; B.143.a.11.i;
B.144.a.11.i; B.145.a.11.i; B.146.a.11.i; B.147.a.11.i; B.148.a.11.i; B.149.a.11.i;
B.150.a.11.i; B.151.a.11.i; B.152.a.11.i; B.153.a.11.i; B.154.a.11.i; B.155.a.11.i;
B.156.a.11.i; B.157.a.11.i; B.158.a.11.i; B.159.a.11.i; B.160.a.11.i; B.161.a.11.i;
B.162.a.11.i; B.163.a.11.i; B.164.a.11.i; B.165.a.11.i; B.166.a.11.i; B.167.a.11.i;
B.168.a.11.i; B.169.a.11.i; B.170.a.11.i; B.171.a.11.i; B.172.a.11.i; B.173.a.11.i;
B.174.a.11.i; B.175.a.11.i; B.176.a.11.i; B.177.a.11.i; B.178.a.11.i; B.179.a.11.i;
B.180.a.11.i; B.181.a.11.i; B.182.a.11.i; B.183.a.11.i; B.184.a.11.i; B.185.a.11.i;
B.186.a.11.i; B.187.a.11.i; B.188.a.11.i; B.189.a.11.i; B.190.a.11.i; B.191.a.11.i;
B.192.a.11.i; B.193.a.11.i; B.194.a.11.i; B.195.a.11.i; B.196.a.11.i; B.197.a.11.i;
B.198.a.11.i; B.199.a.11.i; B.200.a.11.i; B.201.a.11.i; B.202.a.11.i; B.203.a.11.i;
B.204.a.11.i; B.205.a.11.i; B.206.a.11.i; B.207.a.11.i; B.208.a.11.i; B.209.a.11.i;
B.210.a.11.i; B.211.a.11.i; B.212.a.11.i; B.213.a.11.i; B.214.a.11.i; B.215.a.11.i;
B.216.a.11.i; B.217.a.11.i; B.218.a.11.i; B.219.a.11.i; B.220.a.11.i; B.221.a.11.i;
B.222.a.11.i; B.223.a.11.i; B.224.a.11.i; B.225.a.11.i; B.226.a.11.i; B.227.a.11.i;
B.228.a.11.i; B.229.a.11.i; B.230.a.11.i; B.231.a.11.i; B.232.a.11.i; B.233.a.11.i;
B.234.a.11.i; B.235.a.11.i; B.236.a.11.i; B.237.a.11.i; B.238.a.11.i; B.239.a.11.i;
B.240.a.11.i; B.241.a.11.i; B.242.a.11.i; B.243.a.11.i; B.244.a.11.i; B.245.a.11.i;
B.246.a.11.i; B.247.a.11.i; B.248.a.11.i; B.249.a.11.i; B.250.a.11.i; B.251.a.11.i;
B.252.a.11.i; B.253.a.11.i; B.254.a.11.i; B.255.a.11.i; B.256.a.11.i; B.257.a.11.i;
B.258.a.11.i; B.259.a.11.i; B.260.a.11.i; B.261.a.11.i; B.262.a.11.i; B.263.a.11.i;
B.264.a.11.i; B.265.a.11.i; B.266.a.11.i; B.267.a.11.i; B.268.a.11.i; B.269.a.11.i;
B.270.a.11.i; B.271.a.11.i; B.272.a.11.i; B.273.a.11.i; B.274.a.11.i; B.275.a.11.i;
B.276.a.11.i; B.277.a.11.i; B.278.a.11.i; B.279.a.11.i; B.280.a.11.i; B.281.a.11.i;
B.282.a.11.i; B.283.a.11.i; B.284.a.11.i; B.285.a.11.i; B.286.a.11.i; B.287.a.11.i;
B.288.a.11.i; B.289.a.11.i; B.290.a.11.i; B.291.a.11.i; B.292.a.11.i; B.293.a.11.i;
B.294.a.11.i; B.295.a.11.i; B.296.a.11.i; B.297.a.11.i; B.298.a.11.i; B.299.a.11.i;
B.300.a.11.i; B.301.a.11.i; B.302.a.11.i; B.303.a.11.i; B.304.a.11.i; B.305.a.11.i;
B.306.a.11.i; B.307.a.11.i; B.308.a.11.i; B.309.a.11.i; B.310.a.11.i; B.311.a.11.i;
B.312.a.11.i; B.313.a.11.i; B.314.a.11.i; B.315.a.11.i; B.316.a.11.i; B.317.a.11.i;
B.318.a.11.i; B.319.a.11.i; B.320.a.11.i; B.321.a.11.i; B.322.a.11.i; B.323.a.11.i;
B.324.a.11.i; B.325.a.11.i; B.326.a.11.i; B.327.a.11.i; B.328.a.11.i; B.329.a.11.i;
B.330.a.11.i; B.331.a.11.i; B.332.a.11.i; B.333.a.11.i; B.334.a.11.i; B.335.a.11.i;
B.336.a.11.i; B.337.a.11.i; B.338.a.11.i; B.339.a.11.i; B.340.a.11.i; B.341.a.11.i;
B.342.a.11.i; B.343.a.11.i; B.344.a.11.i; B.345.a.11.i; B.346.a.11.i; B.347.a.11.i;
B.348.a.11.i; B.349.a.11.i; B.350.a.11.i; B.351.a.11.i; B.352.a.11.i; B.353.a.11.i;
B.354.a.11.i; B.355.a.11.i; B.356.a.11.i; B.357.a.11.i; B.358.a.11.i; E.2.a.11.i;
E.3.a.11.i; E.4.a.11.i; E.5.a.11.i; E.9.a.11.i; E.100.a.11.i; E.101.a.11.i; E.102.a.11.i;
E.103.a.11.i; E.104.a.11.i; E.105.a.11.i; E.106.a.11.i; E.107.a.11.i; E.108.a.11.i;
E.109.a.11.i; E.110.a.11.i; E.111.a.11.i; E.112.a.11.i; E.113.a.11.i; E.114.a.11.i;
E.115.a.11.i; E.116.a.11.i; E.117.a.11.i; E.118.a.11.i; E.119.a.11.i; E.120.a.11.i;
E.121.a.11.i; E.122.a.11.i; E.123.a.11.i; E.124.a.11.i; E.125.a.11.i; E.126.a.11.i;
E.127.a.11.i; E.128.a.11.i; E.129.a.11.i; E.130.a.11.i; E.131.a.11.i; E.132.a.11.i;
E.133.a.11.i; E.134.a.11.i; E.135.a.11.i; E.136.a.11.i; E.137.a.11.i; E.138.a.11.i;
E.139.a.11.i; E.140.a.11.i; E.141.a.11.i; E.142.a.11.i; E.143.a.11.i; E.144.a.11.i;
E.145.a.11.i; E.146.a.11.i; E.147.a.11.i; E.148.a.11.i; E.149.a.11.i; E.150.a.11.i;
E.151.a.11.i; E.152.a.11.i; E.153.a.11.i; E.154.a.11.i; E.155.a.11.i; E.156.a.11.i;
E.157.a.11.i; E.158.a.11.i; E.159.a.11.i; E.160.a.11.i; E.161.a.11.i; E.162.a.11.i;
E.163.a.11.i; E.164.a.11.i; E.165.a.11.i; E.166.a.11.i; E.167.a.11.i; E.168.a.11.i;
E.169.a.11.i; E.170.a.11.i; E.171.a.11.i; E.172.a.11.i; E.173.a.11.i; E.174.a.11.i;
E.175.a.11.i; E.176.a.11.i; E.177.a.11.i; E.178.a.11.i; E.179.a.11.i; E.180.a.11.i;
E.181.a.11.i; E.182.a.11.i; E.183.a.11.i; E.184.a.11.i; E.185.a.11.i; E.186.a.11.i;
E.187.a.11.i; E.188.a.11.i; E.189.a.11.i; E.190.a.11.i; E.191.a.11.i; E.192.a.11.i;
E.193.a.11.i; E.194.a.11.i; E.195.a.11.i; E.196.a.11.i; E.197.a.11.i; E.198.a.11.i;
E.199.a.11.i; E.200.a.11.i; E.201.a.11.i; E.202.a.11.i; E.203.a.11.i; E.204.a.11.i;
E.205.a.11.i; E.206.a.11.i; E.207.a.11.i; E.208.a.11.i; E.209.a.11.i; E.210.a.11.i;
E.211.a.11.i; E.212.a.11.i; E.213.a.11.i; E.214.a.11.i; E.215.a.11.i; E.216.a.11.i;
E.217.a.11.i; E.218.a.11.i; E.219.a.11.i; E.220.a.11.i; E.221.a.11.i; E.222.a.11.i;
E.223.a.11.i; E.224.a.11.i; E.225.a.11.i; E.226.a.11.i; E.227.a.11.i; E.228.a.11.i;
E.229.a.11.i; E.230.a.11.i; E.231.a.11.i; E.232.a.11.i; E.233.a.11.i; E.234.a.11.i;
E.235.a.11.i; E.236.a.11.i; E.237.a.11.i; E.238.a.11.i; E.239.a.11.i; E.240.a.11.i;
E.241.a.11.i; E.242.a.11.i; E.243.a.11.i; E.244.a.11.i; E.245.a.11.i; E.246.a.11.i;
E.247.a.11.i; E.248.a.11.i; E.249.a.11.i; E.250.a.11.i; E.251.a.11.i; E.252.a.11.i;
E.253.a.11.i; E.254.a.11.i; E.255.a.11.i; E.256.a.11.i; E.257.a.11.i; E.258.a.11.i;
E.259.a.11.i; E.260.a.11.i; E.261.a.11.i; E.262.a.11.i; E.263.a.11.i; E.264.a.11.i;
E.265.a.11.i; E.266.a.11.i; E.267.a.11.i; E.268.a.11.i; E.269.a.11.i; E.270.a.11.i;
E.271.a.11.i; E.272.a.11.i; E.273.a.11.i; E.274.a.11.i; E.275.a.11.i; E.276.a.11.i;
E.277.a.11.i; E.278.a.11.i; E.279.a.11.i; E.280.a.11.i; E.281.a.11.i; E.282.a.11.i;
E.283.a.11.i; E.284.a.11.i; E.285.a.11.i; E.286.a.11.i; E.287.a.11.i; E.288.a.11.i;
E.289.a.11.i; E.290.a.11.i; E.291.a.11.i; E.292.a.11.i; E.293.a.11.i; E.294.a.11.i;
E.295.a.11.i; E.296.a.11.i; E.297.a.11.i; E.298.a.11.i; E.299.a.11.i; E.300.a.11.i;
E.301.a.11.i; E.302.a.11.i; E.303.a.11.i; E.304.a.11.i; E.305.a.11.i; E.306.a.11.i;
E.307.a.11.i; E.308.a.11.i; E.309.a.11.i; E.310.a.11.i; E.311.a.11.i; E.312.a.11.i;
E.313.a.11.i; E.314.a.11.i; E.315.a.11.i; E.316.a.11.i; E.317.a.11.i; E.318.a.11.i;
E.319.a.11.i; E.320.a.11.i; E.321.a.11.i; E.322.a.11.i; E.323.a.11.i; E.324.a.11.i;
E.325.a.11.i; E.326.a.11.i; E.327.a.11.i; E.328.a.11.i; E.329.a.11.i; E.330.a.11.i;
E.331.a.11.i; E.332.a.11.i; E.333.a.11.i; E.334.a.11.i; E.335.a.11.i; E.336.a.11.i;
E.337.a.11.i; E.338.a.11.i; E.339.a.11.i; E.340.a.11.i; E.341.a.11.i; E.342.a.11.i;
E.343.a.11.i; E.344.a.11.i; E.345.a.11.i; E.346.a.11.i; E.347.a.11.i; E.348.a.11.i;
E.349.a.11.i; E.350.a.11.i; E.351.a.11.i; E.352.a.11.i; E.353.a.11.i; E.354.a.11.i;
E.355.a.11.i; E.356.a.11.i; E.357.a.11.i; E.358.a.11.i; A.661.a.4.i; A.662.a.4.i;
A.663.a.4.i; A.664.a.4.i; A.665.a.4.i; B.661.a.4.i; B.662.a.4.i; B.663.a.4.i; B.664.a.4.i;
B.665.a.4.i; C.661.a.4.i; C.662.a.4.i; C.663.a.4.i; C.664.a.4.i; C.665.a.4.i;
A.661.a.11.i; A.662.a.11.i; A.663.a.11.i; A.664.a.11.i; A.665.a.11.i; B.661.a.11.i;
B.662.a.11.i; B.663.a.11.i; B.664.a.11.i; B.665.a.11.i; C.661.a.11.i; C.662.a.11.i;
C.663.a.11.i; C.664.a.11.i; C.665.a.11.i; A.661.a.44.i; A.662.a.44.i; A.663.a.44.i;
A.664.a.44.i; A.665.a.44.i; B.661.a.44.i; B.662.a.44.i; B.663.a.44.i; B.664.a.44.i;
B.665.a.44.i; C.661.a.44.i; C.662.a.44.i; C.663.a.44.i; C.664.a.44.i; C.665.a.44.i;
A.666.a.4.i; A.666.a.11.i; A.666.a.44.i; A.666.b.4.i; A.666.b.11.i; A.666.b.44.i;
A.666.x.4.i; A.666.x.11.i; A.666.x.44.i; A.666.y.4.i; A.666.y.11.i; A.666.y.44.i;
A.666.z.4.i; A.666.z.11.i; A.666.z.44.i; A.666.A.4.i; A.666.A.11.i; A.666.A.44.i;
A.666.B.4.i; A.666.B.11.i; A.666.B.44.i; A.666.C.4.i; A.666.C.11.i; A.666.C.44.i;
A.666.D.4.i; A.666.D.11.i; A.666.D.44.i; A.666.E.4.i; A.666.E.11.i; A.666.E.44.i;
A.666.F.4.i; A.666.F.11.i; A.666.F.44.i; B.666.a.4.i; B.666.a.11.i; B.666.a.44.i;
B.666.b.4.i; B.666.b.11.i; B.666.b.44.i; B.666.x.4.i; B.666.x.11.i; B.666.x.44.i;
B.666.y.4.i; B.666.y.11.i; B.666.y.44.i; B.666.z.4.i; B.666.z.11.i; B.666.z.44.i;
B.666.B.4.i; B.666.B.11.i; B.666.B.44.i; B.666.B.4.i; B.666.B.11.i; B.666.B.44.i;
B.666.C.4.i; B.666.C.11.i; B.666.C.44.i; B.666.D.4.i; B.666.D.11.i; B.666.D.44.i;
B.666.E.4.i; B.666.E.11.i; B.666.E.44.i; B.666.F.4.i; B.666.F.11.i; B.666.F.44.i;
E.666.a.4.i; E.666.a.11.i; E.666.a.44.i; E.666.b.4.i; E.666.b.11.i; E.666.b.44.i;
E.666.x.4.i; E.666.x.11.i; E.666.x.44.i; E.666.y.4.i; E.666.y.11.i; E.666.y.44.i;
E.666.z.4.i; E.666.z.11.i; E.666.z.44.i; E.666.E.4.i; E.666.E.11.i; E.666.E.44.i;
E.666.B.4.i; E.666.B.11.i; E.666.B.44.i; E.666.C.4.i; E.666.C.11.i; E.666.C.44.i;
E.666.D.4.i; E.666.D.11.i; E.666.D.44.i; E.666.E.4.i; E.666.E.11.i; E.666.E.44.i;
E.666.F.4.i; E.666.F.11.i; E.666.F.44.i; A.2.a.46.i; A.3.a.46.i; A.4.a.46.i; A.5.a.46.i;
A.7.a.46.i; A.9.a.46.i; A.100.a.46.i; A.101.a.46.i; A.102.a.46.i; A.103.a.46.i;
A.104.a.46.i; A.105.a.46.i; A.106.a.46.i; A.107.a.46.i; A.108.a.46.i; A.109.a.46.i;
A.110.a.46.i; A.111.a.46.i; A.112.a.46.i; A.113.a.46.i; A.114.a.46.i; A.115.a.46.i;
A.116.a.46.i; A.117.a.46.i; A.118.a.46.i; A.119.a.46.i; A.120.a.46.i; A.121.a.46.i;
A.122.a.46.i; A.123.a.46.i; A.124.a.46.i; A.125.a.46.i; A.126.a.46.i; A.127.a.46.i;
A.128.a.46.i; A.129.a.46.i; A.130.a.46.i; A.131.a.46.i; A.132.a.46.i; A.133.a.46.i;
A.134.a.46.i; A.135.a.46.i; A.136.a.46.i; A.137.a.46.i; A.138.a.46.i; A.139.a.46.i;
A.140.a.46.i; A.141.a.46.i; A.2.a.47.i; A.3.a.47.i; A.4.a.47.i; A.5.a.47.i; A.7.a.47.i;
A.9.a.47.i; A.100.a.47.i; A.101.a.47.i; A.102.a.47.i; A.103.a.47.i; A.104.a.47.i;
A.105.a.47.i; A.106.a.47.i; A.107.a.47.i; A.108.a.47.i; A.109.a.47.i; A.110.a.47.i;
A.111.a.47.i; A.112.a.47.i; A.113.a.47.i; A.114.a.47.i; A.115.a.47.i; A.116.a.47.i;
A.117.a.47.i; A.118.a.47.i; A.119.a.47.i; A.120.a.47.i; A.121.a.47.i; A.122.a.47.i;
A.123.a.47.i; A.124.a.47.i; A.125.a.47.i; A.126.a.47.i; A.127.a.47.i; A.128.a.47.i;
A.129.a.47.i; A.130.a.47.i; A.131.a.47.i; A.132.a.47.i; A.133.a.47.i; A.134.a.47.i;
A.135.a.47.i; A.136.a.47.i; A.137.a.47.i; A.138.a.47.i; A.139.a.47.i; A.140.a.47.i;
A.141.a.47.i; A.2.a.48.i; A.3.a.48.i; A.4.a.48.i; A.5.a.48.i; A.7.a.48.i; A.9.a.48.i;
A.100.a.48.i; A.101.a.48.i; A.102.a.48.i; A.103.a.48.i; A.104.a.48.i; A.105.a.48.i;
A.106.a.48.i; A.107.a.48.i; A.108.a.48.i; A.109.a.48.i; A.110.a.48.i; A.111.a.48.i;
A.112.a.48.i; A.113.a.48.i; A.114.a.48.i; A.115.a.48.i; A.116.a.48.i; A.117.a.48.i;
A.118.a.48.i; A.119.a.48.i; A.120.a.48.i; A.121.a.48.i; A.122.a.48.i; A.123.a.48.i;
A.124.a.48.i; A.125.a.48.i; A.126.a.48.i; A.127.a.48.i; A.128.a.48.i; A.129.a.48.i;
A.130.a.48.i; A.131.a.48.i; A.132.a.48.i; A.133.a.48.i; A.134.a.48.i; A.135.a.48.i;
A.136.a.48.i; A.137.a.48.i; A.138.a.48.i; A.139.a.48.i; A.140.a.48.i; A.141.a.48.i;
A.2.a.49.i; A.3.a.49.i; A.4.a.49.i; A.5.a.49.i; A.7.a.49.i; A.9.a.49.i; A.100.a.49.i;
A.101.a.49.i; A.102.a.49.i; A.103.a.49.i; A.104.a.49.i; A.105.a.49.i; A.106.a.49.i;
A.107.a.49.i; A.108.a.49.i; A.109.a.49.i; A.110.a.49.i; A.111.a.49.i; A.112.a.49.i;
A.113.a.49.i; A.114.a.49.i; A.115.a.49.i; A.116.a.49.i; A.117.a.49.i; A.118.a.49.i;
A.119.a.49.i; A.120.a.49.i; A.121.a.49.i; A.122.a.49.i; A.123.a.49.i; A.124.a.49.i;
A.125.a.49.i; A.126.a.49.i; A.127.a.49.i; A.128.a.49.i; A.129.a.49.i; A.130.a.49.i;
A.131.a.49.i; A.132.a.49.i; A.133.a.49.i; A.134.a.49.i; A.135.a.49.i; A.136.a.49.i;
A.137.a.49.i; A.138.a.49.i; A.139.a.49.i; A.140.a.49.i; A.141.a.49.i; A.2.a.50.i;
A.3.a.50.i; A.4.a.50.i; A.5.a.50.i; A.7.a.50.i; A.9.a.50.i; A.100.a.50.i; A.101.a.50.i;
A.102.a.50.i; A.103.a.50.i; A.104.a.50.i; A.105.a.50.i; A.106.a.50.i; A.107.a.50.i;
A.108.a.50.i; A.109.a.50.i; A.110.a.50.i; A.111.a.50.i; A.112.a.50.i; A.113.a.50.i;
A.114.a.50.i; A.115.a.50.i; A.116.a.50.i; A.117.a.50.i; A.118.a.50.i; A.119.a.50.i;
A.120.a.50.i; A.121.a.50.i; A.122.a.50.i; A.123.a.50.i; A.124.a.50.i; A.125.a.50.i;
A.126.a.50.i; A.127.a.50.i; A.128.a.50.i; A.129.a.50.i; A.130.a.50.i; A.131.a.50.i;
A.132.a.50.i; A.133.a.50.i; A.134.a.50.i; A.135.a.50.i; A.136.a.50.i; A.137.a.50.i;
A.138.a.50.i; A.139.a.50.i; A.140.a.50.i; A.141.a.50.i; A.2.a.51.i; A.3.a.51.i;
A.4.a.51.i; A.5.a.51.i; A.7.a.51.i; A.9.a.51.i; A.100.a.51.i; A.101.a.51.i; A.102.a.51.i;
A.103.a.51.i; A.104.a.51.i; A.105.a.51.i; A.106.a.51.i; A.107.a.51.i; A.108.a.51.i;
A.109.a.51.i; A.110.a.51.i; A.111.a.51.i; A.112.a.51.i; A.113.a.51.i; A.114.a.51.i;
A.115.a.51.i; A.116.a.51.i; A.117.a.51.i; A.118.a.51.i; A.119.a.51.i; A.120.a.51.i;
A.121.a.51.i; A.122.a.51.i; A.123.a.51.i; A.124.a.51.i; A.125.a.51.i; A.126.a.51.i;
A.127.a.51.i; A.128.a.51.i; A.129.a.51.i; A.130.a.51.i; A.131.a.51.i; A.132.a.51.i;
A.133.a.51.i; A.134.a.51.i; A.135.a.51.i; A.136.a.51.i; A.137.a.51.i; A.138.a.51.i;
A.139.a.51.i; A.140.a.51.i; A.141.a.51.i; A.2.b.46.i; A.3.b.46.i; A.4.b.46.i; A.5.b.46.i;
A.7.b.46.i; A.9.b.46.i; A.100.b.46.i; A.101.b.46.i; A.102.b.46.i; A.103.b.46.i;
A.104.b.46.i; A.105.b.46.i; A.106.b.46.i; A.107.b.46.i; A.108.b.46.i; A.109.b.46.i;
A.110.b.46.i; A.111.b.46.i; A.112.b.46.i; A.113.b.46.i; A.114.b.46.i; A.115.b.46.i;
A.116.b.46.i; A.117.b.46.i; A.118.b.46.i; A.119.b.46.i; A.120.b.46.i; A.121.b.46.i;
A.122.b.46.i; A.123.b.46.i; A.124.b.46.i; A.125.b.46.i; A.126.b.46.i; A.127.b.46.i;
A.128.b.46.i; A.129.b.46.i; A.130.b.46.i; A.131.b.46.i; A.132.b.46.i; A.133.b.46.i;
A.134.b.46.i; A.135.b.46.i; A.136.b.46.i; A.137.b.46.i; A.138.b.46.i; A.139.b.46.i;
A.140.b.46.i; A.141.b.47.i; A.2.b.47.i; A.3.b.47.i; A.4.b.47.i; A.5.b.47.i; A.7.b.47.i;
A.9.b.47.i; A.100.b.47.i; A.101.b.47.i; A.102.b.47.i; A.103.b.47.i; A.104.b.47.i;
A.105.b.47.i; A.106.b.47.i; A.107.b.47.i; A.108.b.47.i; A.109.b.47.i; A.110.b.47.i;
A.111.b.47.i; A.112.b.47.i; A.113.b.47.i; A.114.b.47.i; A.115.b.47.i; A.116.b.47.i;
A.117.b.47.i; A.118.b.47.i; A.119.b.47.i; A.120.b.47.i; A.121.b.47.i; A.122.b.47.i;
A.123.b.47.i; A.124.b.47.i; A.125.b.47.i; A.126.b.47.i; A.127.b.47.i; A.128.b.47.i;
A.129.b.47.i; A.130.b.47.i; A.131.b.47.i; A.132.b.47.i; A.133.b.47.i; A.134.b.47.i;
A.135.b.47.i; A.136.b.47.i; A.137.b.47.i; A.138.b.47.i; A.139.b.47.i; A.140.b.47.i;
A.141.b.47.i; A.2.b.48.i; A.3.b.48.i; A.4.b.48.i; A.5.b.48.i; A.7.b.48.i; A.9.b.48.i;
A.100.b.48.i; A.101.b.48.i; A.102.b.48.i; A.103.b.48.i; A.104.b.48.i; A.105.b.48.i;
A.106.b.48.i; A.107.b.48.i; A.108.b.48.i; A.109.b.48.i; A.110.b.48.i; A.111.b.48.i;
A.112.b.48.i; A.113.b.48.i; A.114.b.48.i; A.115.b.48.i; A.116.b.48.i; A.117.b.48.i;
A.118.b.48.i; A.119.b.48.i; A.120.b.48.i; A.121.b.48.i; A.122.b.48.i; A.123.b.48.i;
A.124.b.48.i; A.125.b.48.i; A.126.b.48.i; A.127.b.48.i; A.128.b.48.i; A.129.b.48.i;
A.130.b.48.i; A.131.b.48.i; A.132.b.48.i; A.133.b.48.i; A.134.b.48.i; A.135.b.48.i;
A.136.b.48.i; A.137.b.48.i; A.138.b.48.i; A.139.b.48.i; A.140.b.48.i; A.141.b.48.i;
A.2.b.49.i; A.3.b.49.i; A.4.b.49.i; A.5.b.49.i; A.7.b.49.i; A.9.b.49.i; A.100.b.49.i;
A.101.b.49.i; A.102.b.49.i; A.103.b.49.i; A.104.b.49.i; A.105.b.49.i; A.106.b.49.i;
A.107.b.49.i; A.108.b.49.i; A.109.b.49.i; A.110.b.49.i; A.111.b.49.i; A.112.b.49.i;
A.113.b.49.i; A.114.b.49.i; A.115.b.49.i; A.116.b.49.i; A.117.b.49.i; A.118.b.49.i;
A.119.b.49.i; A.120.b.49.i; A.121.b.49.i; A.122.b.49.i; A.123.b.49.i; A.124.b.49.i;
A.125.b.49.i; A.126.b.49.i; A.127.b.49.i; A.128.b.49.i; A.129.b.49.i; A.130.b.49.i;
A.131.b.49.i; A.132.b.49.i; A.133.b.49.i; A.134.b.49.i; A.135.b.49.i; A.136.b.49.i;
A.137.b.49.i; A.138.b.49.i; A.139.b.49.i; A.140.b.49.i; A.141.b.49.i; A.2.b.50.i;
A.3.b.50.i; A.4.b.50.i; A.5.b.50.i; A.7.b.50.i; A.9.b.50.i; A.100.b.50.i; A.101.b.50.i;
A.102.b.50.i; A.103.b.50.i; A.104.b.50.i; A.105.b.50.i; A.106.b.50.i; A.107.b.50.i;
A.108.b.50.i; A.109.b.50.i; A.110.b.50.i; A.111.b.50.i; A.112.b.50.i; A.113.b.50.i;
A.114.b.50.i; A.115.b.50.i; A.116.b.50.i; A.117.b.50.i; A.118.b.50.i; A.119.b.50.i;
A.120.b.50.i; A.121.b.50.i; A.122.b.50.i; A.123.b.50.i; A.124.b.50.i; A.125.b.50.i;
A.126.b.50.i; A.127.b.50.i; A.128.b.50.i; A.129.b.50.i; A.130.b.50.i; A.131.b.50.i;
A.132.b.50.i; A.133.b.50.i; A.134.b.50.i; A.135.b.50.i; A.136.b.50.i; A.137.b.50.i;
A.138.b.50.i; A.139.b.50.i; A.140.b.50.i; A.141.b.50.i; A.2.b.51.i; A.3.b.51.i;
A.4.b.51.i; A.5.b.51.i; A.7.b.51.i; A.9.b.51.i; A.100.b.51.i; A.101.b.51.i;
A.102.b.51.i; A.103.b.51.i; A.104.b.51.i; A.105.b.51.i; A.106.b.51.i; A.107.b.51.i;
A.108.b.51.i; A.109.b.51.i; A.110.b.51.i; A.111.b.51.i; A.112.b.51.i; A.113.b.51.i;
A.114.b.51.i; A.115.b.51.i; A.116.b.51.i; A.117.b.51.i; A.118.b.51.i; A.119.b.51.i;
A.120.b.51.i; A.121.b.51.i; A.122.b.51.i; A.123.b.51.i; A.124.b.51.i; A.125.b.51.i;
A.126.b.51.i; A.127.b.51.i; A.128.b.51.i; A.129.b.51.i; A.130.b.51.i; A.131.b.51.i;
A.132.b.51.i; A.133.b.51.i; A.134.b.51.i; A.135.b.51.i; A.136.b.51.i; A.137.b.51.i;
A.138.b.51.i; A.139.b.51.i; A.140.b.51.i; A.141.b.51.i; A.2.x.46.i; A.3.x.46.i;
A.4.x.46.i; A.5.x.46.i; A.7.x.46.i; A.9.x.46.i; A.100.x.46.i; A.101.x.46.i; A.102.x.46.i;
A.103.x.46.i; A.104.x.46.i; A.105.x.46.i; A.106.x.46.i; A.107.x.46.i; A.108.x.46.i;
A.109.x.46.i; A.110.x.46.i; A.111.x.46.i; A.112.x.46.i; A.113.x.46.i; A.114.x.46.i;
A.115.x.46.i; A.116.x.46.i; A.117.x.46.i; A.118.x.46.i; A.119.x.46.i; A.120.x.46.i;
A.121.x.46.i; A.122.x.46.i; A.123.x.46.i; A.124.x.46.i; A.125.x.46.i; A.126.x.46.i;
A.127.x.46.i; A.128.x.46.i; A.129.x.46.i; A.130.x.46.i; A.131.x.46.i; A.132.x.46.i;
A.133.x.46.i; A.134.x.46.i; A.135.x.46.i; A.136.x.46.i; A.137.x.46.i; A.138.x.46.i;
A.139.x.46.i; A.140.x.46.i; A.141.x.46.i; A.2.x.47.i; A.3.x.47.i; A.4.x.47.i; A.5.x.47.i;
A.7.x.47.i; A.9.x.47.i; A.100.x.47.i; A.101.x.47.i; A.102.x.47.i; A.103.x.47.i;
A.104.x.47.i; A.105.x.47.i; A.106.x.47.i; A.107.x.47.i; A.108.x.47.i; A.109.x.47.i;
A.110.x.47.i; A.111.x.47.i; A.112.x.47.i; A.113.x.47.i; A.114.x.47.i; A.115.x.47.i;
A.116.x.47.i; A.117.x.47.i; A.118.x.47.i; A.119.x.47.i; A.120.x.47.i; A.121.x.47.i;
A.122.x.47.i; A.123.x.47.i; A.124.x.47.i; A.125.x.47.i; A.126.x.47.i; A.127.x.47.i;
A.128.x.47.i; A.129.x.47.i; A.130.x.47.i; A.131.x.47.i; A.132.x.47.i; A.133.x.47.i;
A.134.x.47.i; A.135.x.47.i; A.136.x.47.i; A.137.x.47.i; A.138.x.47.i; A.139.x.47.i;
A.140.x.47.i; A.141.x.47.i; A.2.x.48.i; A.3.x.48.i; A.4.x.48.i; A.5.x.48.i; A.7.x.48.i;
A.9.x.48.i; A.100.x.48.i; A.101.x.48.i; A.102.x.48.i; A.103.x.48.i; A.104.x.48.i;
A.105.x.48.i; A.106.x.48.i; A.107.x.48.i; A.108.x.48.i; A.109.x.48.i; A.110.x.48.i;
A.111.x.48.i; A.112.x.48.i; A.113.x.48.i; A.114.x.48.i; A.115.x.48.i; A.116.x.48.i;
A.117.x.48.i; A.118.x.48.i; A.119.x.48.i; A.120.x.48.i; A.121.x.48.i; A.122.x.48.i;
A.123.x.48.i; A.124.x.48.i; A.125.x.48.i; A.126.x.48.i; A.127.x.48.i; A.128.x.48.i;
A.129.x.48.i; A.130.x.48.i; A.131.x.48.i; A.132.x.48.i; A.133.x.48.i; A.134.x.48.i;
A.135.x.48.i; A.136.x.48.i; A.137.x.48.i; A.138.x.48.i; A.139.x.48.i; A.140.x.48.i;
A.141.x.48.i; A.2.x.49.i; A.3.x.49.i; A.4.x.49.i; A.5.x.49.i; A.7.x.49.i; A.9.x.49.i;
A.100.x.49.i; A.101.x.49.i; A.102.x.49.i; A.103.x.49.i; A.104.x.49.i; A.105.x.49.i;
A.106.x.49.i; A.107.x.49.i; A.108.x.49.i; A.109.x.49.i; A.110.x.49.i; A.111.x.49.i;
A.112.x.49.i; A.113.x.49.i; A.114.x.49.i; A.115.x.49.i; A.116.x.49.i; A.117.x.49.i;
A.118.x.49.i; A.119.x.49.i; A.120.x.49.i; A.121.x.49.i; A.122.x.49.i; A.123.x.49.i;
A.124.x.49.i; A.125.x.49.i; A.126.x.49.i; A.127.x.49.i; A.128.x.49.i; A.129.x.49.i;
A.130.x.49.i; A.131.x.49.i; A.132.x.49.i; A.133.x.49.i; A.134.x.49.i; A.135.x.49.i;
A.136.x.49.i; A.137.x.49.i; A.138.x.49.i; A.139.x.49.i; A.140.x.49.i; A.141.x.49.i;
A.2.x.50.i; A.3.x.50.i; A.4.x.50.i; A.5.x.50.i; A.7.x.50.i; A.9.x.50.i; A.100.x.50.i;
A.101.x.50.i; A.102.x.50.i; A.103.x.50.i; A.104.x.50.i; A.105.x.50.i; A.106.x.50.i;
A.107.x.50.i; A.108.x.50.i; A.109.x.50.i; A.110.x.50.i; A.111.x.50.i; A.112.x.50.i;
A.113.x.50.i; A.114.x.50.i; A.115.x.50.i; A.116.x.50.i; A.117.x.50.i; A.118.x.50.i;
A.119.x.50.i; A.120.x.50.i; A.121.x.50.i; A.122.x.50.i; A.123.x.50.i; A.124.x.50.i;
A.125.x.50.i; A.126.x.50.i; A.127.x.50.i; A.128.x.50.i; A.129.x.50.i; A.130.x.50.i;
A.131.x.50.i; A.132.x.50.i; A.133.x.50.i; A.134.x.50.i; A.135.x.50.i; A.136.x.50.i;
A.137.x.50.i; A.138.x.50.i; A.139.x.50.i; A.140.x.50.i; A.141.x.50.i; A.2.x.51.i;
A.3.x.51.i; A.4.x.51.i; A.5.x.51.i; A.7.x.51.i; A.9.x.51.i; A.100.x.51.i; A.101.x.51.i;
A.102.x.51.i; A.103.x.51.i; A.104.x.51.i; A.105.x.51.i; A.106.x.51.i; A.107.x.51.i;
A.108.x.51.i; A.109.x.51.i; A.110.x.51.i; A.111.x.51.i; A.112.x.51.i; A.113.x.51.i;
A.114.x.51.i; A.115.x.51.i; A.116.x.51.i; A.117.x.51.i; A.118.x.51.i; A.119.x.51.i;
A.120.x.51.i; A.121.x.51.i; A.122.x.51.i; A.123.x.51.i; A.124.x.51.i; A.125.x.51.i;
A.126.x.51.i; A.127.x.51.i; A.128.x.51.i; A.129.x.51.i; A.130.x.51.i; A.131.x.51.i;
A.132.x.51.i; A.133.x.51.i; A.134.x.51.i; A.135.x.51.i; A.136.x.51.i; A.137.x.51.i;
A.138.x.51.i; A.139.x.51.i; A.140.x.51.i; A.141.x.51.i; A.2.y.46.i; A.3.y.46.i;
A.4.y.46.i; A.5.y.46.i; A.7.y.46.i; A.9.y.46.i; A.100.y.46.i; A.101.y.46.i;
A.102.y.46.i; A.103.y.46.i; A.104.y.46.i; A.105.y.46.i; A.106.y.46.i; A.107.y.46.i;
A.108.y.46.i; A.109.y.46.i; A.110.y.46.i; A.111.y.46.i; A.112.y.46.i; A.113.y.46.i;
A.114.y.46.i; A.115.y.46.i; A.116.y.46.i; A.117.y.46.i; A.118.y.46.i; A.119.y.46.i;
A.120.y.46.i; A.121.y.46.i; A.122.y.46.i; A.123.y.46.i; A.124.y.46.i; A.125.y.46.i;
A.126.y.46.i; A.127.y.46.i; A.128.y.46.i; A.129.y.46.i; A.130.y.46.i; A.131.y.46.i;
A.132.y.46.i; A.133.y.46.i; A.134.y.46.i; A.135.y.46.i; A.136.y.46.i; A.137.y.46.i;
A.138.y.46.i; A.139.y.46.i; A.140.y.46.i; A.141.y.46.i; A.2.y.47.i; A.3.y.47.i;
A.4.y.47.i; A.5.y.47.i; A.7.y.47.i; A.9.y.47.i; A.100.y.47.i; A.101.y.47.i;
A.102.y.47.i; A.103.y.47.i; A.104.y.47.i; A.105.y.47.i; A.106.y.47.i; A.107.y.47.i;
A.108.y.47.i; A.109.y.47.i; A.110.y.47.i; A.111.y.47.i; A.112.y.47.i; A.113.y.47.i;
A.114.y.47.i; A.115.y.47.i; A.116.y.47.i; A.117.y.47.i; A.118.y.47.i; A.119.y.47.i;
A.120.y.47.i; A.121.y.47.i; A.122.y.47.i; A.123.y.47.i; A.124.y.47.i; A.125.y.47.i;
A.126.y.47.i; A.127.y.47.i; A.128.y.47.i; A.129.y.47.i; A.130.y.47.i; A.131.y.47.i;
A.132.y.47.i; A.133.y.47.i; A.134.y.47.i; A.135.y.47.i; A.136.y.47.i; A.137.y.47.i;
A.138.y.47.i; A.139.y.47.i; A.140.y.47.i; A.141.y.47.i; A.2.y.48.i; A.3.y.48.i;
A.4.y.48.i; A.5.y.48.i; A.7.y.48.i; A.9.y.48.i; A.100.y.48.i; A.101.y.48.i;
A.102.y.48.i; A.103.y.48.i; A.104.y.48.i; A.105.y.48.i; A.106.y.48.i; A.107.y.48.i;
A.108.y.48.i; A.109.y.48.i; A.110.y.48.i; A.111.y.48.i; A.112.y.48.i; A.113.y.48.i;
A.114.y.48.i; A.115.y.48.i; A.116.y.48.i; A.117.y.48.i; A.118.y.48.i; A.119.y.48.i;
A.120.y.48.i; A.121.y.48.i; A.122.y.48.i; A.123.y.48.i; A.124.y.48.i; A.125.y.48.i;
A.126.y.48.i; A.127.y.48.i; A.128.y.48.i; A.129.y.48.i; A.130.y.48.i; A.131.y.48.i;
A.132.y.48.i; A.133.y.48.i; A.134.y.48.i; A.135.y.48.i; A.136.y.48.i; A.137.y.48.i;
A.138.y.48.i; A.139.y.48.i; A.140.y.48.i; A.141.y.48.i; A.2.y.49.i; A.3.y.49.i;
A.4.y.49.i; A.5.y.49.i; A.7.y.49.i; A.9.y.49.i; A.100.y.49.i; A.101.y.49.i;
A.102.y.49.i; A.103.y.49.i; A.104.y.49.i; A.105.y.49.i; A.106.y.49.i; A.107.y.49.i;
A.108.y.49.i; A.109.y.49.i; A.110.y.49.i; A.111.y.49.i; A.112.y.49.i; A.113.y.49.i;
A.114.y.49.i; A.115.y.49.i; A.116.y.49.i; A.117.y.49.i; A.118.y.49.i; A.119.y.49.i;
A.120.y.49.i; A.121.y.49.i; A.122.y.49.i; A.123.y.49.i; A.124.y.49.i; A.125.y.49.i;
A.126.y.49.i; A.127.y.49.i; A.128.y.49.i; A.129.y.49.i; A.130.y.49.i; A.131.y.49.i;
A.132.y.49.i; A.133.y.49.i; A.134.y.49.i; A.135.y.49.i; A.136.y.49.i; A.137.y.49.i;
A.138.y.49.i; A.139.y.49.i; A.140.y.49.i; A.141.y.49.i; A.2.y.50.i; A.3.y.50.i;
A.4.y.50.i; A.5.y.50.i; A.7.y.50.i; A.9.y.50.i; A.100.y.50.i; A.101.y.50.i;
A.102.y.50.i; A.103.y.50.i; A.104.y.50.i; A.105.y.50.i; A.106.y.50.i; A.107.y.50.i;
A.108.y.50.i; A.109.y.50.i; A.110.y.50.i; A.111.y.50.i; A.112.y.50.i; A.113.y.50.i;
A.114.y.50.i; A.115.y.50.i; A.116.y.50.i; A.117.y.50.i; A.118.y.50.i; A.119.y.50.i;
A.120.y.50.i; A.121.y.50.i; A.122.y.50.i; A.123.y.50.i; A.124.y.50.i; A.125.y.50.i;
A.126.y.50.i; A.127.y.50.i; A.128.y.50.i; A.129.y.50.i; A.130.y.50.i; A.131.y.50.i;
A.132.y.50.i; A.133.y.50.i; A.134.y.50.i; A.135.y.50.i; A.136.y.50.i; A.137.y.50.i;
A.138.y.50.i; A.139.y.50.i; A.140.y.50.i; A.141.y.50.i; A.2.y.51.i; A.3.y.51.i;
A.4.y.51.i; A.5.y.51.i; A.7.y.51.i; A.9.y.51.i; A.100.y.51.i; A.101.y.51.i;
A.102.y.51.i; A.103.y.51.i; A.104.y.51.i; A.105.y.51.i; A.106.y.51.i; A.107.y.51.i;
A.108.y.51.i; A.109.y.51.i; A.110.y.51.i; A.111.y.51.i; A.112.y.51.i; A.113.y.51.i;
A.114.y.51.i; A.115.y.51.i; A.116.y.51.i; A.117.y.51.i; A.118.y.51.i; A.119.y.51.i;
A.120.y.51.i; A.121.y.51.i; A.122.y.51.i; A.123.y.51.i; A.124.y.51.i; A.125.y.51.i;
A.126.y.51.i; A.127.y.51.i; A.128.y.51.i; A.129.y.51.i; A.130.y.51.i; A.131.y.51.i;
A.132.y.51.i; A.133.y.51.i; A.134.y.51.i; A.135.y.51.i; A.136.y.51.i; A.137.y.51.i;
A.138.y.51.i; A.139.y.51.i; A.140.y.51.i; A.141.y.51.i; A.2.z.46.i; A.3.z.46.i;
A.4.z.46.i; A.5.z.46.i; A.7.z.46.i; A.9.z.46.i; A.100.z.46.i; A.101.z.46.i; A.102.z.46.i;
A.103.z.46.i; A.104.z.46.i; A.105.z.46.i; A.106.z.46.i; A.107.z.46.i; A.108.z.46.i;
A.109.z.46.i; A.110.z.46.i; A.111.z.46.i; A.112.z.46.i; A.113.z.46.i; A.114.z.46.i;
A.115.z.46.i; A.116.z.46.i; A.117.z.46.i; A.118.z.46.i; A.119.z.46.i; A.120.z.46.i;
A.121.z.46.i; A.122.z.46.i; A.123.z.46.i; A.124.z.46.i; A.125.z.46.i; A.126.z.46.i;
A.127.z.46.i; A.128.z.46.i; A.129.z.46.i; A.130.z.46.i; A.131.z.46.i; A.132.z.46.i;
A.133.z.46.i; A.134.z.46.i; A.135.z.46.i; A.136.z.46.i; A.137.z.46.i; A.138.z.46.i;
A.139.z.46.i; A.140.z.46.i; A.141.z.46.i; A.2.z.47.i; A.3.z.47.i; A.4.z.47.i; A.5.z.47.i;
A.7.z.47.i; A.9.z.47.i; A.100.z.47.i; A.101.z.47.i; A.102.z.47.i; A.103.z.47.i;
A.104.z.47.i; A.105.z.47.i; A.106.z.47.i; A.107.z.47.i; A.108.z.47.i; A.109.z.47.i;
A.110.z.47.i; A.111.z.47.i; A.112.z.47.i; A.113.z.47.i; A.114.z.47.i; A.115.z.47.i;
A.116.z.47.i; A.117.z.47.i; A.118.z.47.i; A.119.z.47.i; A.120.z.47.i; A.121.z.47.i;
A.122.z.47.i; A.123.z.47.i; A.124.z.47.i; A.125.z.47.i; A.126.z.47.i; A.127.z.47.i;
A.128.z.47.i; A.129.z.47.i; A.130.z.47.i; A.131.z.47.i; A.132.z.47.i; A.133.z.47.i;
A.134.z.47.i; A.135.z.47.i; A.136.z.47.i; A.137.z.47.i; A.138.z.47.i; A.139.z.47.i;
A.140.z.47.i; A.141.z.47.i; A.2.z.48.i; A.3.z.48.i; A.4.z.48.i; A.5.z.48.i; A.7.z.48.i;
A.9.z.48.i; A.100.z.48.i; A.101.z.48.i; A.102.z.48.i; A.103.z.48.i; A.104.z.48.i;
A.105.z.48.i; A.106.z.48.i; A.107.z.48.i; A.108.z.48.i; A.109.z.48.i; A.110.z.48.i;
A.111.z.48.i; A.112.z.48.i; A.113.z.48.i; A.114.z.48.i; A.115.z.48.i; A.116.z.48.i;
A.117.z.48.i; A.118.z.48.i; A.119.z.48.i; A.120.z.48.i; A.121.z.48.i; A.122.z.48.i;
A.123.z.48.i; A.124.z.48.i; A.125.z.48.i; A.126.z.48.i; A.127.z.48.i; A.128.z.48.i;
A.129.z.48.i; A.130.z.48.i; A.131.z.48.i; A.132.z.48.i; A.133.z.48.i; A.134.z.48.i;
A.135.z.48.i; A.136.z.48.i; A.137.z.48.i; A.138.z.48.i; A.139.z.48.i; A.140.z.48.i;
A.141.z.48.i; A.2.z.49.i; A.3.z.49.i; A.4.z.49.i; A.5.z.49.i; A.7.z.49.i; A.9.z.49.i;
A.100.z.49.i; A.101.z.49.i; A.102.z.49.i; A.103.z.49.i; A.104.z.49.i; A.105.z.49.i;
A.106.z.49.i; A.107.z.49.i; A.108.z.49.i; A.109.z.49.i; A.110.z.49.i; A.111.z.49.i;
A.112.z.49.i; A.113.z.49.i; A.114.z.49.i; A.115.z.49.i; A.116.z.49.i; A.117.z.49.i;
A.118.z.49.i; A.119.z.49.i; A.120.z.49.i; A.121.z.49.i; A.122.z.49.i; A.123.z.49.i;
A.124.z.49.i; A.125.z.49.i; A.126.z.49.i; A.127.z.49.i; A.128.z.49.i; A.129.z.49.i;
A.130.z.49.i; A.131.z.49.i; A.132.z.49.i; A.133.z.49.i; A.134.z.49.i; A.135.z.49.i;
A.136.z.49.i; A.137.z.49.i; A.138.z.49.i; A.139.z.49.i; A.140.z.49.i; A.141.z.49.i;
A.2.z.50.i; A.3.z.50.i; A.4.z.50.i; A.5.z.50.i; A.7.z.50.i; A.9.z.50.i; A.100.z.50.i;
A.101.z.50.i; A.102.z.50.i; A.103.z.50.i; A.104.z.50.i; A.105.z.50.i; A.106.z.50.i;
A.107.z.50.i; A.108.z.50.i; A.109.z.50.i; A.110.z.50.i; A.111.z.50.i; A.112.z.50.i;
A.113.z.50.i; A.114.z.50.i; A.115.z.50.i; A.116.z.50.i; A.117.z.50.i; A.118.z.50.i;
A.119.z.50.i; A.120.z.50.i; A.121.z.50.i; A.122.z.50.i; A.123.z.50.i; A.124.z.50.i;
A.125.z.50.i; A.126.z.50.i; A.127.z.50.i; A.128.z.50.i; A.129.z.50.i; A.130.z.50.i;
A.131.z.50.i; A.132.z.50.i; A.133.z.50.i; A.134.z.50.i; A.135.z.50.i; A.136.z.50.i;
A.137.z.50.i; A.138.z.50.i; A.139.z.50.i; A.140.z.50.i; A.141.z.50.i; A.2.z.51.i;
A.3.z.51.i; A.4.z.51.i; A.5.z.51.i; A.7.z.51.i; A.9.z.51.i; A.100.z.51.i; A.101.z.51.i;
A.102.z.51.i; A.103.z.51.i; A.104.z.51.i; A.105.z.51.i; A.106.z.51.i; A.107.z.51.i;
A.108.z.51.i; A.109.z.51.i; A.110.z.51.i; A.111.z.51.i; A.112.z.51.i; A.113.z.51.i;
A.114.z.51.i; A.115.z.51.i; A.116.z.51.i; A.117.z.51.i; A.118.z.51.i; A.119.z.51.i;
A.120.z.51.i; A.121.z.51.i; A.122.z.51.i; A.123.z.51.i; A.124.z.51.i; A.125.z.51.i;
A.126.z.51.i; A.127.z.51.i; A.128.z.51.i; A.129.z.51.i; A.130.z.51.i; A.131.z.51.i;
A.132.z.51.i; A.133.z.51.i; A.134.z.51.i; A.135.z.51.i; A.136.z.51.i; A.137.z.51.i;
A.138.z.51.i; A.139.z.51.i; A.140.z.51.i; A.141.z.51.i; A.2.A.46.i; A.3.A.46.i;
A.4.A.46.i; A.5.A.46.i; A.7.A.46.i; A.9.A.46.i; A.100.A.46.i; A.101.A.46.i;
A.102.A.46.i; A.103.A.46.i; A.104.A.46.i; A.105.A.46.i; A.106.A.46.i; A.107.A.46.i;
A.108.A.46.i; A.109.A.46.i; A.110.A.46.i; A.111.A.46.i; A.112.A.46.i; A.113.A.46.i;
A.114.A.46.i; A.115.A.46.i; A.116.A.46.i; A.117.A.46.i; A.118.A.46.i; A.119.A.46.i;
A.120.A.46.i; A.121.A.46.i; A.122.A.46.i; A.123.A.46.i; A.124.A.46.i; A.125.A.46.i;
A.126.A.46.i; A.127.A.46.i; A.128.A.46.i; A.129.A.46.i; A.130.A.46.i; A.131.A.46.i;
A.132.A.46.i; A.133.A.46.i; A.134.A.46.i; A.135.A.46.i; A.136.A.46.i; A.137.A.46.i;
A.138.A.46.i; A.139.A.46.i; A.140.A.46.i; A.141.A.46.i; A.2.A.47.i; A.3.A.47.i;
A.4.A.47.i; A.5.A.47.i; A.7.A.47.i; A.9.A.47.i; A.100.A.47.i; A.101.A.47.i;
A.102.A.47.i; A.103.A.47.i; A.104.A.47.i; A.105.A.47.i; A.106.A.47.i; A.107.A.47.i;
A.108.A.47.i; A.109.A.47.i; A.110.A.47.i; A.111.A.47.i; A.112.A.47.i; A.113.A.47.i;
A.114.A.47.i; A.115.A.47.i; A.116.A.47.i; A.117.A.47.i; A.118.A.47.i; A.119.A.47.i;
A.120.A.47.i; A.121.A.47.i; A.122.A.47.i; A.123.A.47.i; A.124.A.47.i; A.125.A.47.i;
A.126.A.47.i; A.127.A.47.i; A.128.A.47.i; A.129.A.47.i; A.130.A.47.i; A.131.A.47.i;
A.132.A.47.i; A.133.A.47.i; A.134.A.47.i; A.135.A.47.i; A.136.A.47.i; A.137.A.47.i;
A.138.A.47.i; A.139.A.47.i; A.140.A.47.i; A.141.A.47.i; A.2.A.48.i; A.3.A.48.i;
A.4.A.48.i; A.5.A.48.i; A.7.A.48.i; A.9.A.48.i; A.100.A.48.i; A.101.A.48.i;
A.102.A.48.i; A.103.A.48.i; A.104.A.48.i; A.105.A.48.i; A.106.A.48.i; A.107.A.48.i;
A.108.A.48.i; A.109.A.48.i; A.110.A.48.i; A.111.A.48.i; A.112.A.48.i; A.113.A.48.i;
A.114.A.48.i; A.115.A.48.i; A.116.A.48.i; A.117.A.48.i; A.118.A.48.i; A.119.A.48.i;
A.120.A.48.i; A.121.A.48.i; A.122.A.48.i; A.123.A.48.i; A.124.A.48.i; A.125.A.48.i;
A.126.A.48.i; A.127.A.48.i; A.128.A.48.i; A.129.A.48.i; A.130.A.48.i; A.131.A.48.i;
A.132.A.48.i; A.133.A.48.i; A.134.A.48.i; A.135.A.48.i; A.136.A.48.i; A.137.A.48.i;
A.138.A.48.i; A.139.A.48.i; A.140.A.48.i; A.141.A.48.i; A.2.A.49.i; A.3.A.49.i;
A.4.A.49.i; A.5.A.49.i; A.7.A.49.i; A.9.A.49.i; A.100.A.49.i; A.101.A.49.i;
A.102.A.49.i; A.103.A.49.i; A.104.A.49.i; A.105.A.49.i; A.106.A.49.i; A.107.A.49.i;
A.108.A.49.i; A.109.A.49.i; A.110.A.49.i; A.111.A.49.i; A.112.A.49.i; A.113.A.49.i;
A.114.A.49.i; A.115.A.49.i; A.116.A.49.i; A.117.A.49.i; A.118.A.49.i; A.119.A.49.i;
A.120.A.49.i; A.121.A.49.i; A.122.A.49.i; A.123.A.49.i; A.124.A.49.i; A.125.A.49.i;
A.126.A.49.i; A.127.A.49.i; A.128.A.49.i; A.129.A.49.i; A.130.A.49.i; A.131.A.49.i;
A.132.A.49.i; A.133.A.49.i; A.134.A.49.i; A.135.A.49.i; A.136.A.49.i; A.137.A.49.i;
A.138.A.49.i; A.139.A.49.i; A.140.A.49.i; A.141.A.49.i; A.2.A.50.i; A.3.A.50.i;
A.4.A.50.i; A.5.A.50.i; A.7.A.50.i; A.9.A.50.i; A.100.A.50.i; A.101.A.50.i;
A.102.A.50.i; A.103.A.50.i; A.104.A.50.i; A.105.A.50.i; A.106.A.50.i; A.107.A.50.i;
A.108.A.50.i; A.109.A.50.i; A.110.A.50.i; A.111.A.50.i; A.112.A.50.i; A.113.A.50.i;
A.114.A.50.i; A.115.A.50.i; A.116.A.50.i; A.117.A.50.i; A.118.A.50.i; A.119.A.50.i;
A.120.A.50.i; A.121.A.50.i; A.122.A.50.i; A.123.A.50.i; A.124.A.50.i; A.125.A.50.i;
A.126.A.50.i; A.127.A.50.i; A.128.A.50.i; A.129.A.50.i; A.130.A.50.i; A.131.A.50.i;
A.132.A.50.i; A.133.A.50.i; A.134.A.50.i; A.135.A.50.i; A.136.A.50.i; A.137.A.50.i;
A.138.A.50.i; A.139.A.50.i; A.140.A.50.i; A.141.A.50.i; A.2.A.51.i; A.3.A.51.i;
A.4.A.51.i; A.5.A.51.i; A.7.A.51.i; A.9.A.51.i; A.100.A.51.i; A.101.A.51.i;
A.102.A.51.i; A.103.A.51.i; A.104.A.51.i; A.105.A.51.i; A.106.A.51.i; A.107.A.51.i;
A.108.A.51.i; A.109.A.51.i; A.110.A.51.i; A.111.A.51.i; A.112.A.51.i; A.113.A.51.i;
A.114.A.51.i; A.115.A.51.i; A.116.A.51.i; A.117.A.51.i; A.118.A.51.i; A.119.A.51.i;
A.120.A.51.i; A.121.A.51.i; A.122.A.51.i; A.123.A.51.i; A.124.A.51.i; A.125.A.51.i;
A.126.A.51.i; A.127.A.51.i; A.128.A.51.i; A.129.A.51.i; A.130.A.51.i; A.131.A.51.i;
A.132.A.51.i; A.133.A.51.i; A.134.A.51.i; A.135.A.51.i; A.136.A.51.i; A.137.A.51.i;
A.138.A.51.i; A.139.A.51.i; A.140.A.51.i; A.141.A.51.i; A.2.B.46.i; A.3.B.46.i;
A.4.B.46.i; A.5.B.46.i; A.7.B.46.i; A.9.B.46.i; A.100.B.46.i; A.101.B.46.i;
A.102.B.46.i; A.103.B.46.i; A.104.B.46.i; A.105.B.46.i; A.106.B.46.i; A.107.B.46.i;
A.108.B.46.i; A.109.B.46.i; A.110.B.46.i; A.111.B.46.i; A.112.B.46.i; A.113.B.46.i;
A.114.B.46.i; A.115.B.46.i; A.116.B.46.i; A.117.B.46.i; A.118.B.46.i; A.119.B.46.i;
A.120.B.46.i; A.121.B.46.i; A.122.B.46.i; A.123.B.46.i; A.124.B.46.i; A.125.B.46.i;
A.126.B.46.i; A.127.B.46.i; A.128.B.46.i; A.129.B.46.i; A.130.B.46.i; A.131.B.46.i;
A.132.B.46.i; A.133.B.46.i; A.134.B.46.i; A.135.B.46.i; A.136.B.46.i; A.137.B.46.i;
A.138.B.46.i; A.139.B.46.i; A.140.B.46.i; A.141.B.46.i; A.2.B.47.i; A.3.B.47.i;
A.4.B.47.i; A.5.B.47.i; A.7.B.47.i; A.9.B.47.i; A.100.B.47.i; A.101.B.47.i;
A.102.B.47.i; A.103.B.47.i; A.104.B.47.i; A.105.B.47.i; A.106.B.47.i; A.107.B.47.i;
A.108.B.47.i; A.109.B.47.i; A.110.B.47.i; A.111.B.47.i; A.112.B.47.i; A.113.B.47.i;
A.114.B.47.i; A.115.B.47.i; A.116.B.47.i; A.117.B.47.i; A.118.B.47.i; A.119.B.47.i;
A.120.B.47.i; A.121.B.47.i; A.122.B.47.i; A.123.B.47.i; A.124.B.47.i; A.125.B.47.i;
A.126.B.47.i; A.127.B.47.i; A.128.B.47.i; A.129.B.47.i; A.130.B.47.i; A.131.B.47.i;
A.132.B.47.i; A.133.B.47.i; A.134.B.47.i; A.135.B.47.i; A.136.B.47.i; A.137.B.47.i;
A.138.B.47.i; A.139.B.47.i; A.140.B.47.i; A.141.B.47.i; A.2.B.48.i; A.3.B.48.i;
A.4.B.48.i; A.5.B.48.i; A.7.B.48.i; A.9.B.48.i; A.100.B.48.i; A.101.B.48.i;
A.102.B.48.i; A.103.B.48.i; A.104.B.48.i; A.105.B.48.i; A.106.B.48.i; A.107.B.48.i;
A.108.B.48.i; A.109.B.48.i; A.110.B.48.i; A.111.B.48.i; A.112.B.48.i; A.113.B.48.i;
A.114.B.48.i; A.115.B.48.i; A.116.B.48.i; A.117.B.48.i; A.118.B.48.i; A.119.B.48.i;
A.120.B.48.i; A.121.B.48.i; A.122.B.48.i; A.123.B.48.i; A.124.B.48.i; A.125.B.48.i;
A.126.B.48.i; A.127.B.48.i; A.128.B.48.i; A.129.B.48.i; A.130.B.48.i; A.131.B.48.i;
A.132.B.48.i; A.133.B.48.i; A.134.B.48.i; A.135.B.48.i; A.136.B.48.i; A.137.B.48.i;
A.138.B.48.i; A.139.B.48.i; A.140.B.48.i; A.141.B.48.i; A.2.B.49.i; A.3.B.49.i;
A.4.B.49.i; A.5.B.49.i; A.7.B.49.i; A.9.B.49.i; A.100.B.49.i; A.101.B.49.i;
A.102.B.49.i; A.103.B.49.i; A.104.B.49.i; A.105.B.49.i; A.106.B.49.i; A.107.B.49.i;
A.108.B.49.i; A.109.B.49.i; A.110.B.49.i; A.111.B.49.i; A.112.B.49.i; A.113.B.49.i;
A.114.B.49.i; A.115.B.49.i; A.116.B.49.i; A.117.B.49.i; A.118.B.49.i; A.119.B.49.i;
A.120.B.49.i; A.121.B.49.i; A.122.B.49.i; A.123.B.49.i; A.124.B.49.i; A.125.B.49.i;
A.126.B.49.i; A.127.B.49.i; A.128.B.49.i; A.129.B.49.i; A.130.B.49.i; A.131.B.49.i;
A.132.B.49.i; A.133.B.49.i; A.134.B.49.i; A.135.B.49.i; A.136.B.49.i; A.137.B.49.i;
A.138.B.49.i; A.139.B.49.i; A.140.B.49.i; A.141.B.49.i; A.2.B.50.i; A.3.B.50.i;
A.4.B.50.i; A.5.B.50.i; A.7.B.50.i; A.9.B.50.i; A.100.B.50.i; A.101.B.50.i;
A.102.B.50.i; A.103.B.50.i; A.104.B.50.i; A.105.B.50.i; A.106.B.50.i; A.107.B.50.i;
A.108.B.50.i; A.109.B.50.i; A.110.B.50.i; A.111.B.50.i; A.112.B.50.i; A.113.B.50.i;
A.114.B.50.i; A.115.B.50.i; A.116.B.50.i; A.117.B.50.i; A.118.B.50.i; A.119.B.50.i;
A.120.B.50.i; A.121.B.50.i; A.122.B.50.i; A.123.B.50.i; A.124.B.50.i; A.125.B.50.i;
A.126.B.50.i; A.127.B.50.i; A.128.B.50.i; A.129.B.50.i; A.130.B.50.i; A.131.B.50.i;
A.132.B.50.i; A.133.B.50.i; A.134.B.50.i; A.135.B.50.i; A.136.B.50.i; A.137.B.50.i;
A.138.B.50.i; A.139.B.50.i; A.140.B.50.i; A.141.B.50.i; A.2.B.51.i; A.3.B.51.i;
A.4.B.51.i; A.5.B.51.i; A.7.B.51.i; A.9.B.51.i; A.100.B.51.i; A.101.B.51.i;
A.102.B.51.i; A.103.B.51.i; A.104.B.51.i; A.105.B.51.i; A.106.B.51.i; A.107.B.51.i;
A.108.B.51.i; A.109.B.51.i; A.110.B.51.i; A.111.B.51.i; A.112.B.51.i; A.113.B.51.i;
A.114.B.51.i; A.115.B.51.i; A.116.B.51.i; A.117.B.51.i; A.118.B.51.i; A.119.B.51.i;
A.120.B.51.i; A.121.B.51.i; A.122.B.51.i; A.123.B.51.i; A.124.B.51.i; A.125.B.51.i;
A.126.B.51.i; A.127.B.51.i; A.128.B.51.i; A.129.B.51.i; A.130.B.51.i; A.131.B.51.i;
A.132.B.51.i; A.133.B.51.i; A.134.B.51.i; A.135.B.51.i; A.136.B.51.i; A.137.B.51.i;
A.138.B.51.i; A.139.B.51.i; A.140.B.51.i; A.141.B.51.i; A.2.C.46.i; A.3.C.46.i;
A.4.C.46.i; A.5.C.46.i; A.7.C.46.i; A.9.C.46.i; A.100.C.46.i; A.101.C.46.i;
A.102.C.46.i; A.103.C.46.i; A.104.C.46.i; A.105.C.46.i; A.106.C.46.i; A.107.C.46.i;
A.108.C.46.i; A.109.C.46.i; A.110.C.46.i; A.111.C.46.i; A.112.C.46.i; A.113.C.46.i;
A.114.C.46.i; A.115.C.46.i; A.116.C.46.i; A.117.C.46.i; A.118.C.46.i; A.119.C.46.i;
A.120.C.46.i; A.121.C.46.i; A.122.C.46.i; A.123.C.46.i; A.124.C.46.i; A.125.C.46.i;
A.126.C.46.i; A.127.C.46.i; A.128.C.46.i; A.129.C.46.i; A.130.C.46.i; A.131.C.46.i;
A.132.C.46.i; A.133.C.46.i; A.134.C.46.i; A.135.C.46.i; A.136.C.46.i; A.137.C.46.i;
A.138.C.46.i; A.139.C.46.i; A.140.C.46.i; A.141.C.46.i; A.2.C.47.i; A.3.C.47.i;
A.4.C.47.i; A.5.C.47.i; A.7.C.47.i; A.9.C.47.i; A.100.C.47.i; A.101.C.47.i;
A.102.C.47.i; A.103.C.47.i; A.104.C.47.i; A.105.C.47.i; A.106.C.47.i; A.107.C.47.i;
A.108.C.47.i; A.109.C.47.i; A.110.C.47.i; A.111.C.47.i; A.112.C.47.i; A.113.C.47.i;
A.114.C.47.i; A.115.C.47.i; A.116.C.47.i; A.117.C.47.i; A.118.C.47.i; A.119.C.47.i;
A.120.C.47.i; A.121.C.47.i; A.122.C.47.i; A.123.C.47.i; A.124.C.47.i; A.125.C.47.i;
A.126.C.47.i; A.127.C.47.i; A.128.C.47.i; A.129.C.47.i; A.130.C.47.i; A.131.C.47.i;
A.132.C.47.i; A.133.C.47.i; A.134.C.47.i; A.135.C.47.i; A.136.C.47.i; A.137.C.47.i;
A.138.C.47.i; A.139.C.47.i; A.140.C.47.i; A.141.C.47.i; A.2.C.48.i; A.3.C.48.i;
A.4.C.48.i; A.5.C.48.i; A.7.C.48.i; A.9.C.48.i; A.100.C.48.i; A.101.C.48.i;
A.102.C.48.i; A.103.C.48.i; A.104.C.48.i; A.105.C.48.i; A.106.C.48.i; A.107.C.48.i;
A.108.C.48.i; A.109.C.48.i; A.110.C.48.i; A.111.C.48.i; A.112.C.48.i; A.113.C.48.i;
A.114.C.48.i; A.115.C.48.i; A.116.C.48.i; A.117.C.48.i; A.118.C.48.i; A.119.C.48.i;
A.120.C.48.i; A.121.C.48.i; A.122.C.48.i; A.123.C.48.i; A.124.C.48.i; A.125.C.48.i;
A.126.C.48.i; A.127.C.48.i; A.128.C.48.i; A.129.C.48.i; A.130.C.48.i; A.131.C.48.i;
A.132.C.48.i; A.133.C.48.i; A.134.C.48.i; A.135.C.48.i; A.136.C.48.i; A.137.C.48.i;
A.138.C.48.i; A.139.C.48.i; A.140.C.48.i; A.141.C.48.i; A.2.C.49.i; A.3.C.49.i;
A.4.C.49.i; A.5.C.49.i; A.7.C.49.i; A.9.C.49.i; A.100.C.49.i; A.101.C.49.i;
A.102.C.49.i; A.103.C.49.i; A.104.C.49.i; A.105.C.49.i; A.106.C.49.i; A.107.C.49.i;
A.108.C.49.i; A.109.C.49.i; A.110.C.49.i; A.111.C.49.i; A.112.C.49.i; A.113.C.49.i;
A.114.C.49.i; A.115.C.49.i; A.116.C.49.i; A.117.C.49.i; A.118.C.49.i; A.119.C.49.i;
A.120.C.49.i; A.121.C.49.i; A.122.C.49.i; A.123.C.49.i; A.124.C.49.i; A.125.C.49.i;
A.126.C.49.i; A.127.C.49.i; A.128.C.49.i; A.129.C.49.i; A.130.C.49.i; A.131.C.49.i;
A.132.C.49.i; A.133.C.49.i; A.134.C.49.i; A.135.C.49.i; A.136.C.49.i; A.137.C.49.i;
A.138.C.49.i; A.139.C.49.i; A.140.C.49.i; A.141.C.49.i; A.2.C.50.i; A.3.C.50.i;
A.4.C.50.i; A.5.C.50.i; A.7.C.50.i; A.9.C.50.i; A.100.C.50.i; A.101.C.50.i;
A.102.C.50.i; A.103.C.50.i; A.104.C.50.i; A.105.C.50.i; A.106.C.50.i; A.107.C.50.i;
A.108.C.50.i; A.109.C.50.i; A.110.C.50.i; A.111.C.50.i; A.112.C.50.i; A.113.C.50.i;
A.114.C.50.i; A.115.C.50.i; A.116.C.50.i; A.117.C.50.i; A.118.C.50.i; A.119.C.50.i;
A.120.C.50.i; A.121.C.50.i; A.122.C.50.i; A.123.C.50.i; A.124.C.50.i; A.125.C.50.i;
A.126.C.50.i; A.127.C.50.i; A.128.C.50.i; A.129.C.50.i; A.130.C.50.i; A.131.C.50.i;
A.132.C.50.i; A.133.C.50.i; A.134.C.50.i; A.135.C.50.i; A.136.C.50.i; A.137.C.50.i;
A.138.C.50.i; A.139.C.50.i; A.140.C.50.i; A.141.C.50.i; A.2.C.51.i; A.3.C.51.i;
A.4.C.51.i; A.5.C.51.i; A.7.C.51.i; A.9.C.51.i; A.100.C.51.i; A.101.C.51.i;
A.102.C.51.i; A.103.C.51.i; A.104.C.51.i; A.105.C.51.i; A.106.C.51.i; A.107.C.51.i;
A.108.C.51.i; A.109.C.51.i; A.110.C.51.i; A.111.C.51.i; A.112.C.51.i; A.113.C.51.i;
A.114.C.51.i; A.115.C.51.i; A.116.C.51.i; A.117.C.51.i; A.118.C.51.i; A.119.C.51.i;
A.120.C.51.i; A.121.C.51.i; A.122.C.51.i; A.123.C.51.i; A.124.C.51.i; A.125.C.51.i;
A.126.C.51.i; A.127.C.51.i; A.128.C.51.i; A.129.C.51.i; A.130.C.51.i; A.131.C.51.i;
A.132.C.51.i; A.133.C.51.i; A.134.C.51.i; A.135.C.51.i; A.136.C.51.i; A.137.C.51.i;
A.138.C.51.i; A.139.C.51.i; A.140.C.51.i; A.141.C.51.i; A.2.D.46.i; A.3.D.46.i;
A.4.D.46.i; A.5.D.46.i; A.7.D.46.i; A.9.D.46.i; A.100.D.46.i; A.101.D.46.i;
A.102.D.46.i; A.103.D.46.i; A.104.D.46.i; A.105.D.46.i; A.106.D.46.i; A.107.D.46.i;
A.108.D.46.i; A.109.D.46.i; A.110.D.46.i; A.111.D.46.i; A.112.D.46.i; A.113.D.46.i;
A.114.D.46.i; A.115.D.46.i; A.116.D.46.i; A.117.D.46.i; A.118.D.46.i; A.119.D.46.i;
A.120.D.46.i; A.121.D.46.i; A.122.D.46.i; A.123.D.46.i; A.124.D.46.i; A.125.D.46.i;
A.126.D.46.i; A.127.D.46.i; A.128.D.46.i; A.129.D.46.i; A.130.D.46.i; A.131.D.46.i;
A.132.D.46.i; A.133.D.46.i; A.134.D.46.i; A.135.D.46.i; A.136.D.46.i; A.137.D.46.i;
A.138.D.46.i; A.139.D.46.i; A.140.D.46.i; A.141.D.46.i; A.2.D.47.i; A.3.D.47.i;
A.4.D.47.i; A.5.D.47.i; A.7.D.47.i; A.9.D.47.i; A.100.D.47.i; A.101.D.47.i;
A.102.D.47.i; A.103.D.47.i; A.104.D.47.i; A.105.D.47.i; A.106.D.47.i; A.107.D.47.i;
A.108.D.47.i; A.109.D.47.i; A.110.D.47.i; A.111.D.47.i; A.112.D.47.i; A.113.D.47.i;
A.114.D.47.i; A.115.D.47.i; A.116.D.47.i; A.117.D.47.i; A.118.D.47.i; A.119.D.47.i;
A.120.D.47.i; A.121.D.47.i; A.122.D.47.i; A.123.D.47.i; A.124.D.47.i; A.125.D.47.i;
A.126.D.47.i; A.127.D.47.i; A.128.D.47.i; A.129.D.47.i; A.130.D.47.i; A.131.D.47.i;
A.132.D.47.i; A.133.D.47.i; A.134.D.47.i; A.135.D.47.i; A.136.D.47.i; A.137.D.47.i;
A.138.D.47.i; A.139.D.47.i; A.140.D.47.i; A.141.D.47.i; A.2.D.48.i; A.3.D.48.i;
A.4.D.48.i; A.5.D.48.i; A.7.D.48.i; A.9.D.48.i; A.100.D.48.i; A.101.D.48.i;
A.102.D.48.i; A.103.D.48.i; A.104.D.48.i; A.105.D.48.i; A.106.D.48.i; A.107.D.48.i;
A.108.D.48.i; A.109.D.48.i; A.110.D.48.i; A.111.D.48.i; A.112.D.48.i; A.113.D.48.i;
A.114.D.48.i; A.115.D.48.i; A.116.D.48.i; A.117.D.48.i; A.118.D.48.i; A.119.D.48.i;
A.120.D.48.i; A.121.D.48.i; A.122.D.48.i; A.123.D.48.i; A.124.D.48.i; A.125.D.48.i;
A.126.D.48.i; A.127.D.48.i; A.128.D.48.i; A.129.D.48.i; A.130.D.48.i; A.131.D.48.i;
A.132.D.48.i; A.133.D.48.i; A.134.D.48.i; A.135.D.48.i; A.136.D.48.i; A.137.D.48.i;
A.138.D.48.i; A.139.D.48.i; A.140.D.48.i; A.141.D.48.i; A.2.D.49.i; A.3.D.49.i;
A.4.D.49.i; A.5.D.49.i; A.7.D.49.i; A.9.D.49.i; A.100.D.49.i; A.101.D.49.i;
A.102.D.49.i; A.103.D.49.i; A.104.D.49.i; A.105.D.49.i; A.106.D.49.i; A.107.D.49.i;
A.108.D.49.i; A.109.D.49.i; A.110.D.49.i; A.111.D.49.i; A.112.D.49.i; A.113.D.49.i;
A.114.D.49.i; A.115.D.49.i; A.116.D.49.i; A.117.D.49.i; A.118.D.49.i; A.119.D.49.i;
A.120.D.49.i; A.121.D.49.i; A.122.D.49.i; A.123.D.49.i; A.124.D.49.i; A.125.D.49.i;
A.126.D.49.i; A.127.D.49.i; A.128.D.49.i; A.129.D.49.i; A.130.D.49.i; A.131.D.49.i;
A.132.D.49.i; A.133.D.49.i; A.134.D.49.i; A.135.D.49.i; A.136.D.49.i; A.137.D.49.i;
A.138.D.49.i; A.139.D.49.i; A.140.D.49.i; A.141.D.49.i; A.2.D.50.i; A.3.D.50.i;
A.4.D.50.i; A.5.D.50.i; A.7.D.50.i; A.9.D.50.i; A.100.D.50.i; A.101.D.50.i;
A.102.D.50.i; A.103.D.50.i; A.104.D.50.i; A.105.D.50.i; A.106.D.50.i; A.107.D.50.i;
A.108.D.50.i; A.109.D.50.i; A.110.D.50.i; A.111.D.50.i; A.112.D.50.i; A.113.D.50.i;
A.114.D.50.i; A.115.D.50.i; A.116.D.50.i; A.117.D.50.i; A.118.D.50.i; A.119.D.50.i;
A.120.D.50.i; A.121.D.50.i; A.122.D.50.i; A.123.D.50.i; A.124.D.50.i; A.125.D.50.i;
A.126.D.50.i; A.127.D.50.i; A.128.D.50.i; A.129.D.50.i; A.130.D.50.i; A.131.D.50.i;
A.132.D.50.i; A.133.D.50.i; A.134.D.50.i; A.135.D.50.i; A.136.D.50.i; A.137.D.50.i;
A.138.D.50.i; A.139.D.50.i; A.140.D.50.i; A.141.D.50.i; A.2.D.51.i; A.3.D.51.i;
A.4.D.51.i; A.5.D.51.i; A.7.D.51.i; A.9.D.51.i; A.100.D.51.i; A.101.D.51.i;
A.102.D.51.i; A.103.D.51.i; A.104.D.51.i; A.105.D.51.i; A.106.D.51.i; A.107.D.51.i;
A.108.D.51.i; A.109.D.51.i; A.110.D.51.i; A.111.D.51.i; A.112.D.51.i; A.113.D.51.i;
A.114.D.51.i; A.115.D.51.i; A.116.D.51.i; A.117.D.51.i; A.118.D.51.i; A.119.D.51.i;
A.120.D.51.i; A.121.D.51.i; A.122.D.51.i; A.123.D.51.i; A.124.D.51.i; A.125.D.51.i;
A.126.D.51.i; A.127.D.51.i; A.128.D.51.i; A.129.D.51.i; A.130.D.51.i; A.131.D.51.i;
A.132.D.51.i; A.133.D.51.i; A.134.D.51.i; A.135.D.51.i; A.136.D.51.i; A.137.D.51.i;
A.138.D.51.i; A.139.D.51.i; A.140.D.51.i; A.141.D.51.i; A.2.E.46.i; A.3.E.46.i;
A.4.E.46.i; A.5.E.46.i; A.7.E.46.i; A.9.E.46.i; A.100.E.46.i; A.101.E.46.i;
A.102.E.46.i; A.103.E.46.i; A.104.E.46.i; A.105.E.46.i; A.106.E.46.i; A.107.E.46.i;
A.108.E.46.i; A.109.E.46.i; A.110.E.46.i; A.111.E.46.i; A.112.E.46.i; A.113.E.46.i;
A.114.E.46.i; A.115.E.46.i; A.116.E.46.i; A.117.E.46.i; A.118.E.46.i; A.119.E.46.i;
A.120.E.46.i; A.121.E.46.i; A.122.E.46.i; A.123.E.46.i; A.124.E.46.i; A.125.E.46.i;
A.126.E.46.i; A.127.E.46.i; A.128.E.46.i; A.129.E.46.i; A.130.E.46.i; A.131.E.46.i;
A.132.E.46.i; A.133.E.46.i; A.134.E.46.i; A.135.E.46.i; A.136.E.46.i; A.137.E.46.i;
A.138.E.46.i; A.139.E.46.i; A.140.E.46.i; A.141.E.46.i; A.2.E.47.i; A.3.E.47.i;
A.4.E.47.i; A.5.E.47.i; A.7.E.47.i; A.9.E.47.i; A.100.E.47.i; A.101.E.47.i;
A.102.E.47.i; A.103.E.47.i; A.104.E.47.i; A.105.E.47.i; A.106.E.47.i; A.107.E.47.i;
A.108.E.47.i; A.109.E.47.i; A.110.E.47.i; A.111.E.47.i; A.112.E.47.i; A.113.E.47.i;
A.114.E.47.i; A.115.E.47.i; A.116.E.47.i; A.117.E.47.i; A.118.E.47.i; A.119.E.47.i;
A.120.E.47.i; A.121.E.47.i; A.122.E.47.i; A.123.E.47.i; A.124.E.47.i; A.125.E.47.i;
A.126.E.47.i; A.127.E.47.i; A.128.E.47.i; A.129.E.47.i; A.130.E.47.i; A.131.E.47.i;
A.132.E.47.i; A.133.E.47.i; A.134.E.47.i; A.135.E.47.i; A.136.E.47.i; A.137.E.47.i;
A.138.E.47.i; A.139.E.47.i; A.140.E.47.i; A.141.E.47.i; A.2.E.48.i; A.3.E.48.i;
A.4.E.48.i; A.5.E.48.i; A.7.E.48.i; A.9.E.48.i; A.100.E.48.i; A.101.E.48.i;
A.102.E.48.i; A.103.E.48.i; A.104.E.48.i; A.105.E.48.i; A.106.E.48.i; A.107.E.48.i;
A.108.E.48.i; A.109.E.48.i; A.110.E.48.i; A.111.E.48.i; A.112.E.48.i; A.113.E.48.i;
A.114.E.48.i; A.115.E.48.i; A.116.E.48.i; A.117.E.48.i; A.118.E.48.i; A.119.E.48.i;
A.120.E.48.i; A.121.E.48.i; A.122.E.48.i; A.123.E.48.i; A.124.E.48.i; A.125.E.48.i;
A.126.E.48.i; A.127.E.48.i; A.128.E.48.i; A.129.E.48.i; A.130.E.48.i; A.131.E.48.i;
A.132.E.48.i; A.133.E.48.i; A.134.E.48.i; A.135.E.48.i; A.136.E.48.i; A.137.E.48.i;
A.138.E.48.i; A.139.E.48.i; A.140.E.48.i; A.141.E.48.i; A.2.E.49.i; A.3.E.49.i;
A.4.E.49.i; A.5.E.49.i; A.7.E.49.i; A.9.E.49.i; A.100.E.49.i; A.101.E.49.i;
A.102.E.49.i; A.103.E.49.i; A.104.E.49.i; A.105.E.49.i; A.106.E.49.i; A.107.E.49.i;
A.108.E.49.i; A.109.E.49.i; A.110.E.49.i; A.111.E.49.i; A.112.E.49.i; A.113.E.49.i;
A.114.E.49.i; A.115.E.49.i; A.116.E.49.i; A.117.E.49.i; A.118.E.49.i; A.119.E.49.i;
A.120.E.49.i; A.121.E.49.i; A.122.E.49.i; A.123.E.49.i; A.124.E.49.i; A.125.E.49.i;
A.126.E.49.i; A.127.E.49.i; A.128.E.49.i; A.129.E.49.i; A.130.E.49.i; A.131.E.49.i;
A.132.E.49.i; A.133.E.49.i; A.134.E.49.i; A.135.E.49.i; A.136.E.49.i; A.137.E.49.i;
A.138.E.49.i; A.139.E.49.i; A.140.E.49.i; A.141.E.49.i; A.2.E.50.i; A.3.E.50.i;
A.4.E.50.i; A.5.E.50.i; A.7.E.50.i; A.9.E.50.i; A.100.E.50.i; A.101.E.50.i;
A.102.E.50.i; A.103.E.50.i; A.104.E.50.i; A.105.E.50.i; A.106.E.50.i; A.107.E.50.i;
A.108.E.50.i; A.109.E.50.i; A.110.E.50.i; A.111.E.50.i; A.112.E.50.i; A.113.E.50.i;
A.114.E.50.i; A.115.E.50.i; A.116.E.50.i; A.117.E.50.i; A.118.E.50.i; A.119.E.50.i;
A.120.E.50.i; A.121.E.50.i; A.122.E.50.i; A.123.E.50.i; A.124.E.50.i; A.125.E.50.i;
A.126.E.50.i; A.127.E.50.i; A.128.E.50.i; A.129.E.50.i; A.130.E.50.i; A.131.E.50.i;
A.132.E.50.i; A.133.E.50.i; A.134.E.50.i; A.135.E.50.i; A.136.E.50.i; A.137.E.50.i;
A.138.E.50.i; A.139.E.50.i; A.140.E.50.i; A.141.E.50.i; A.2.E.51.i; A.3.E.51.i;
A.4.E.51.i; A.5.E.51.i; A.7.E.51.i; A.9.E.51.i; A.100.E.51.i; A.101.E.51.i;
A.102.E.51.i; A.103.E.51.i; A.104.E.51.i; A.105.E.51.i; A.106.E.51.i; A.107.E.51.i;
A.108.E.51.i; A.109.E.51.i; A.110.E.51.i; A.111.E.51.i; A.112.E.51.i; A.113.E.51.i;
A.114.E.51.i; A.115.E.51.i; A.116.E.51.i; A.117.E.51.i; A.118.E.51.i; A.119.E.51.i;
A.120.E.51.i; A.121.E.51.i; A.122.E.51.i; A.123.E.51.i; A.124.E.51.i; A.125.E.51.i;
A.126.E.51.i; A.127.E.51.i; A.128.E.51.i; A.129.E.51.i; A.130.E.51.i; A.131.E.51.i;
A.132.E.51.i; A.133.E.51.i; A.134.E.51.i; A.135.E.51.i; A.136.E.51.i; A.137.E.51.i;
A.138.E.51.i; A.139.E.51.i; A.140.E.51.i; A.141.E.51.i; A.2.F.46.i; A.3.F.46.i;
A.4.F.46.i; A.5.F.46.i; A.7.F.46.i; A.9.F.46.i; A.100.F.46.i; A.101.F.46.i;
A.102.F.46.i; A.103.F.46.i; A.104.F.46.i; A.105.F.46.i; A.106.F.46.i; A.107.F.46.i;
A.108.F.46.i; A.109.F.46.i; A.110.F.46.i; A.111.F.46.i; A.112.F.46.i; A.113.F.46.i;
A.114.F.46.i; A.115.F.46.i; A.116.F.46.i; A.117.F.46.i; A.118.F.46.i; A.119.F.46.i;
A.120.F.46.i; A.121.F.46.i; A.122.F.46.i; A.123.F.46.i; A.124.F.46.i; A.125.F.46.i;
A.126.F.46.i; A.127.F.46.i; A.128.F.46.i; A.129.F.46.i; A.130.F.46.i; A.131.F.46.i;
A.132.F.46.i; A.133.F.46.i; A.134.F.46.i; A.135.F.46.i; A.136.F.46.i; A.137.F.46.i;
A.138.F.46.i; A.139.F.46.i; A.140.F.46.i; A.141.F.46.i; A.2.F.47.i; A.3.F.47.i;
A.4.F.47.i; A.5.F.47.i; A.7.F.47.i; A.9.F.47.i; A.100.F.47.i; A.101.F.47.i;
A.102.F.47.i; A.103.F.47.i; A.104.F.47.i; A.105.F.47.i; A.106.F.47.i; A.107.F.47.i;
A.108.F.47.i; A.109.F.47.i; A.110.F.47.i; A.111.F.47.i; A.112.F.47.i; A.113.F.47.i;
A.114.F.47.i; A.115.F.47.i; A.116.F.47.i; A.117.F.47.i; A.118.F.47.i; A.119.F.47.i;
A.120.F.47.i; A.121.F.47.i; A.122.F.47.i; A.123.F.47.i; A.124.F.47.i; A.125.F.47.i;
A.126.F.47.i; A.127.F.47.i; A.128.F.47.i; A.129.F.47.i; A.130.F.47.i; A.131.F.47.i;
A.132.F.47.i; A.133.F.47.i; A.134.F.47.i; A.135.F.47.i; A.136.F.47.i; A.137.F.47.i;
A.138.F.47.i; A.139.F.47.i; A.140.F.47.i; A.141.F.47.i; A.2.F.48.i; A.3.F.48.i;
A.4.F.48.i; A.5.F.48.i; A.7.F.48.i; A.9.F.48.i; A.100.F.48.i; A.101.F.48.i;
A.102.F.48.i; A.103.F.48.i; A.104.F.48.i; A.105.F.48.i; A.106.F.48.i; A.107.F.48.i;
A.108.F.48.i; A.109.F.48.i; A.110.F.48.i; A.111.F.48.i; A.112.F.48.i; A.113.F.48.i;
A.114.F.48.i; A.115.F.48.i; A.116.F.48.i; A.117.F.48.i; A.118.F.48.i; A.119.F.48.i;
A.120.F.48.i; A.121.F.48.i; A.122.F.48.i; A.123.F.48.i; A.124.F.48.i; A.125.F.48.i;
A.126.F.48.i; A.127.F.48.i; A.128.F.48.i; A.129.F.48.i; A.130.F.48.i; A.131.F.48.i;
A.132.F.48.i; A.133.F.48.i; A.134.F.48.i; A.135.F.48.i; A.136.F.48.i; A.137.F.48.i;
A.138.F.48.i; A.139.F.48.i; A.140.F.48.i; A.141.F.48.i; A.2.F.49.i; A.3.F.49.i;
A.4.F.49.i; A.5.F.49.i; A.7.F.49.i; A.9.F.49.i; A.100.F.49.i; A.101.F.49.i;
A.102.F.49.i; A.103.F.49.i; A.104.F.49.i; A.105.F.49.i; A.106.F.49.i; A.107.F.49.i;
A.108.F.49.i; A.109.F.49.i; A.110.F.49.i; A.111.F.49.i; A.112.F.49.i; A.113.F.49.i;
A.114.F.49.i; A.115.F.49.i; A.116.F.49.i; A.117.F.49.i; A.118.F.49.i; A.119.F.49.i;
A.120.F.49.i; A.121.F.49.i; A.122.F.49.i; A.123.F.49.i; A.124.F.49.i; A.125.F.49.i;
A.126.F.49.i; A.127.F.49.i; A.128.F.49.i; A.129.F.49.i; A.130.F.49.i; A.131.F.49.i;
A.132.F.49.i; A.133.F.49.i; A.134.F.49.i; A.135.F.49.i; A.136.F.49.i; A.137.F.49.i;
A.138.F.49.i; A.139.F.49.i; A.140.F.49.i; A.141.F.49.i; A.2.F.50.i; A.3.F.50.i;
A.4.F.50.i; A.5.F.50.i; A.7.F.50.i; A.9.F.50.i; A.100.F.50.i; A.101.F.50.i;
A.102.F.50.i; A.103.F.50.i; A.104.F.50.i; A.105.F.50.i; A.106.F.50.i; A.107.F.50.i;
A.108.F.50.i; A.109.F.50.i; A.110.F.50.i; A.111.F.50.i; A.112.F.50.i; A.113.F.50.i;
A.114.F.50.i; A.115.F.50.i; A.116.F.50.i; A.117.F.50.i; A.118.F.50.i; A.119.F.50.i;
A.120.F.50.i; A.121.F.50.i; A.122.F.50.i; A.123.F.50.i; A.124.F.50.i; A.125.F.50.i;
A.126.F.50.i; A.127.F.50.i; A.128.F.50.i; A.129.F.50.i; A.130.F.50.i; A.131.F.50.i;
A.132.F.50.i; A.133.F.50.i; A.134.F.50.i; A.135.F.50.i; A.136.F.50.i; A.137.F.50.i;
A.138.F.50.i; A.139.F.50.i; A.140.F.50.i; A.141.F.50.i; A.2.F.51.i; A.3.F.51.i;
A.4.F.51.i; A.5.F.51.i; A.7.F.51.i; A.9.F.51.i; A.100.F.51.i; A.101.F.51.i;
A.102.F.51.i; A.103.F.51.i; A.104.F.51.i; A.105.F.51.i; A.106.F.51.i; A.107.F.51.i;
A.108.F.51.i; A.109.F.51.i; A.110.F.51.i; A.111.F.51.i; A.112.F.51.i; A.113.F.51.i;
A.114.F.51.i; A.115.F.51.i; A.116.F.51.i; A.117.F.51.i; A.118.F.51.i; A.119.F.51.i;
A.120.F.51.i; A.121.F.51.i; A.122.F.51.i; A.123.F.51.i; A.124.F.51.i; A.125.F.51.i;
A.126.F.51.i; A.127.F.51.i; A.128.F.51.i; A.129.F.51.i; A.130.F.51.i; A.131.F.51.i;
A.132.F.51.i; A.133.F.51.i; A.134.F.51.i; A.135.F.51.i; A.136.F.51.i; A.137.F.51.i;
A.138.F.51.i; A.139.F.51.i; A.140.F.51.i; A.141.F.51.i;

Salts and Hydrates

The compositions of this invention optionally comprise salts of the compounds herein, especially pharmaceutically acceptable non-toxic salts containing, for example, Na⁺, Li⁺, K⁺, Ca⁺⁺ and Mg⁺⁺. Such salts may include those derived by combination of appropriate cations such as alkali and alkaline earth metal ions or ammonium and quaternary amino ions with an acid anion moiety, typically the W₁ group carboxylic acid. Monovalent salts are preferred if a water soluble salt is desired.

Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound.

In addition, salts may be formed from acid addition of certain organic and inorganic acids, e.g., HCl, HBr, H₂SO₄, H₃PO₄, or organic sulfonic acids, to basic centers, typically amines of group G₁, or to acidic groups such as E₁. Finally, it is to be understood that the compositions herein comprise compounds of the invention in their un-ionized, as well as zwitterionic form, and combinations with stoiochimetric amounts of water as in hydrates.

Also included within the scope of this invention are the salts of the parental compounds with one or more amino acids. Any of the amino acids described above are suitable, especially the naturally-occurring amino acids found as protein components, although the amino acid typically is one bearing a side chain with a basic or acidic group, e.g., lysine, arginine or glutamic acid, or a neutral group such as glycine, serine, threonine, alanine, isoleucine, or leucine.

### Methods of Inhibition of Neuraminidase

Another aspect of the invention relates to methods of inhibiting the activity of neuraminidase comprising the step of treating a sample suspected of containing neuraminidase with a compound of the invention.

Compositions of the invention act as inhibitors of neuraminidase, as intermediates for such inhibitors or have other utilities as described below. The inhibitors will bind to locations on the surface or in a cavity of neuraminidase having a geometry unique to neuraminidase. Compositions binding neuraminidase may bind with varying degrees of reversibility. Those compounds binding substantially irreversibly are ideal candidates for use in this method of the invention. In a typical embodiment the compositions bind neuraminidase with a binding coefficient of less than 10⁻⁴M, more typically less than 10⁻⁶M, still more typically 10⁻⁸M. Once labeled, the substantially irreversibly binding compositions are useful as probes for the detection of neuraminidase. Accordingly, the invention relates to methods of detecting neuraminidase in a sample suspected of containing neuraminidase comprising the steps of: treating a sample suspected of containing neuraminidase with a composition comprising a compound of the invention bound to a label; and observing the effect of the sample on the activity of the label. Suitable labels are well known in the diagnostics field and include stable free radicals, fluorophores, radioisotopes, enzymes, chemiluminescent groups and chromogens. The compounds herein are labeled in conventional fashion using functional groups such as hydroxyl or amino.

Within the context of the invention samples suspected of containing neuraminidase include natural or man-made materials such as living organisms; tissue or cell cultures; biological samples such as biological material samples (blood, serum, urine, cerebrospinal fluid, tears, sputum, saliva, tissue samples, and the like); laboratory samples; food, water, or air samples; bioproduct samples such as extracts of cells, particularly recombinant cells synthesizing a desired glycoprotein; and the like. Typically the sample will be suspected of containing an organism which produces neuraminidase, frequently a pathogenic organism such as a virus. Samples can be contained in any medium including water and organic solvent/water mixtures. Samples include living organisms such as humans, and man made materials such as cell cultures.

The treating step of the invention comprises adding the composition of the invention to the sample or it comprises adding a precursor of the composition to the sample. The addition step comprises any method of administration as described above.

If desired, the activity of neuraminidase after application of the composition can be observed by any method including direct and indirect methods of detecting neuraminidase activity. Quantitative, qualitative, and semiquantitative methods of determining neuraminidase activity are all contemplated. Typically one of the screening methods described above are applied, however, any other method such as observation of the physiological properties of a living organism are also applicable.

Organisms that contain neuraminidase include bacteria (Vibrio cholerae, Clostridium perfringens, Streptococcus pneumoniae, and Arthrobacter sialophilus) and viruses (especially orthomyxoviruses or paramyxoviruses such as influenza virus A and B, parainfluenza virus, mumps virus, Newcastle disease virus, fowl plague virus, and sendai virus). Inhibition of neuraminidase activity obtained from or found within any of these organisms is within the objects of this invention. The virology of influenza viruses is described in “Fundamental Virology” (Raven Press, New York, 1986), Chapter 24. The compounds of this invention are useful in the treatment or prophylaxis of such infections in animals, e.g. duck, rodents, or swine, or in man.

However, in screening compounds capable of inhibiting influenza viruses it should be kept in mind that the results of enzyme assays may not correlate with cell culture assays, as shown Table 1 of Chandler et al., supra. Thus, a plaque reduction assay should be the primary screening tool.

### Screens for Neuraminidase Inhibitors

Compositions of the invention are screened for inhibitory activity against neuraminidase by any of the conventional techniques for evaluating enzyme activity. Within the context of the invention, typically compositions are first screened for inhibition of neuraminidase in vitro and compositions showing inhibitory activity are then screened for activity in vivo. Compositions having in vitro K₁ (inhibitory constants) of less then about 5×10⁻⁶ M, typically less than about 1×10⁻⁷ M and preferably less than about 5×10⁻⁸ M are preferred for in vivo use.

Useful in vitro screens have been described in detail and will not be elaborated here. However, von Itzstein, M. et al.; “Nature”, 363(6428):418-423 (1993), in particular page 420, column 2, full paragraph 3, to page 421, column 2, first partial paragraph, describes a suitable in vitro assay of Potier, M.; et al.; “Analyt. Biochem.”, 94:287-296 (1979), as modified by Chong, A. K. J.; et al.; “Biochem. Biophys. Acta”, 1077:65-71 (1991); and Colman, P. M.; et al.; International Publication No. WO 92/06691 (Int. App. No. PCT/AU90/00501, publication date Apr. 30, 1992) page 34, line 13, to page 35, line 16, describes another useful in vitro screen.

In vivo screens have also been described in detail, see von Itzstein, M. et al.; op. cit., in particular page 421, column 2, first full paragraph, to page 423, column 2, first partial paragraph, and Colman, P. M.; et al.; op. cit. page 36, lines 1-38, describe suitable in vivo screens.

### Pharmaceutical Formulations and Routes of Administration

The compounds of this invention are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the “Handbook of Pharmaceutical Excipients” (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

One or more compounds of the invention (herein referred to as the active ingredients) are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this invention is that they are orally bioavailable and can be dosed orally; it is not necessary to administer them by intrapulmonary or intranasal routes. Surprisingly, (in view of, inter alia, Bamford, M. J., “J. Enzyme Inhibition” 10:1-6 (1995), and especially p. 15, first full paragraph), the anti-influenza compounds of WO 91/16320, WO 92/06691 and U.S. Pat. No. 5,360,817 are successfully administered by the oral or intraperitoneal routes. See Example 161 infra.

While it is possible for the active ingredients to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the invention suitable for oral administration are prepared as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom. In one embodiment acid hydrolysis of the medicament is obviated by use of an enteric coating.

For infections of the eye or other external tissues e.g. mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of influenza A or B infections as described below.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention are used to provide controlled release pharmaceutical formulations containing as active ingredient one or more compounds of the invention (“controlled release formulations”) in which the release of the active ingredient are controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active influenza infection, the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, for inhalation the daily candidate dose for an adult human of approximately 70 kg body weight will range from 1 mg to 1000 mg, preferably between 5 mg and 500 mg, and may take the form of single or multiple doses.

Typical doses include 5, 10, 15, 20, 25, 30, 35, 40, 45, 50, 55, 60, 65, 70, 75, 80, 85, 90, 95, 100, 110, 115, 120, 125, 130, 135, 140, 145, 150, 157, 200, 225, 250, 275, 300, 325, 350, 375, 400, 425, 450, 475, 500, 550, 600, 650, 700, 750, 800, 850, 900, 950, and 1000 mg of GS 4104, phosphate salt, once or twice a day; more typically, 20, 25, 30, 35, 40, 45, 50, 55, 60, 65, 70, 75, 80, 85, 90, 95, 100, 110, 115, 120, 125, 130, 135, 140, 145, 150, 157, 200 mg of GS 4104, phosphate salt, once or twice a day; more typically still 20, 50, 75, 100, 150 and 200 mg of GS 4104, phosphate salt, once or twice a day; more typically yet 75 or 150 mg of GS 4104, phosphate salt, once or twice a day.

Active ingredients of the invention are also used in combination with other active ingredients. Such combinations are selected based on the condition to be treated, cross-reactivities of ingredients and pharmaco-properties of the combination. For example, when treating viral infections of the respiratory system, in particular influenza infection, the compositions of the invention are combined with antivirals (such as amantidine, rimantadine and ribavirin), mucolytics, expectorants, bronchialdilators, antibiotics, antipyretics, or analgesics. Ordinarily, antibiotics, antipyretics, and analgesics are administered together with the compounds of this invention.

### Metabolites of the Compounds of the Invention

Also falling within the scope of this invention are the in vivo metabolic products of the compounds described herein, to the extent such products are novel and unobvious over the prior art. Such products may result for example from the oxidation, reduction, hydrolysis, amidation, esterification and the like of the administered compound, primarily due to enzymatic processes. Accordingly, the invention includes novel and unobvious compounds produced by a process comprising contacting a compound of this invention with a mammal for a period of time sufficient to yield a metabolic product thereof. Such products typically are identified by preparing a radiolabelled (e.g. C¹⁴ or H³) compound of the invention, administering it parenterally in a detectable dose (e.g. greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g. by MS or NMR analysis. In general, analysis of metabolites is done in the same way as conventional drug metabolism studies well-known to those skilled in the art. The conversion products, so long as they are not otherwise found in vivo, are useful in diagnostic assays for therapeutic dosing of the compounds of the invention even if they possess no neuraminidase inhibitory activity of their own.

### Additional Uses for the Compounds of This Invention

The compounds of this invention, or the biologically active substances produced from these compounds by hydrolysis or metabolism in vivo, are used as immunogens or for conjugation to proteins, whereby they serve as components of immunogenic compositions to prepare antibodies capable of binding specifically to the protein, to the compounds or to their metabolic products which retain immunologically recognized epitopes (sites of antibody binding). The immunogenic compositions therefore are useful as intermediates in the preparation of antibodies for use in diagnostic, quality control, or the like, methods or in assays for the compounds or their novel metabolic products. The compounds are useful for raising antibodies against otherwise non-immunogenic polypeptides, in that the compounds serve as haptenic sites stimulating an immune response that cross-reacts with the unmodified conjugated protein.

The hydrolysis products of interest include products of the hydrolysis of the protected acidic and basic groups discussed above. As noted above, the acidic or basic amides comprising immunogenic polypeptides such as albumin or keyhole limpet hemocyanin generally are useful as immunogens. The metabolic products described above may retain a substantial degree of immunological cross reactivity with the compounds of the invention. Thus, the antibodies of this invention will be capable of binding to the unprotected compounds of the invention without binding to the protected compounds; alternatively the metabolic products, will be capable of binding to the protected compounds and/or the metabolitic products without binding to the protected compounds of the invention, or will be capable of binding specifically to any one or all three. The antibodies desirably will not substantially cross-react with naturally-occurring materials. Substantial cross-reactivity is reactivity under specific assay conditions for specific analytes sufficient to interfere with the assay results.

The immunogens of this invention contain the compound of this invention presenting the desired epitope in association with an immunogenic substance. Within the context of the invention such association means covalent bonding to form an immunogenic conjugate (when applicable) or a mixture of non-covalently bonded materials, or a combination of the above. Immunogenic substances include adjuvants such as Freund's adjuvant, immunogenic proteins such as viral, bacterial, yeast, plant and animal polypeptides, in particular keyhole limpet hemocyanin, serum albumin, bovine thyroglobulin or soybean trypsin inhibitor, and immunogenic polysaccharides. Typically, the compound having the structure of the desired epitope is covalently conjugated to an immunogenic polypeptide or polysaccharide by the use of a polyfunctional (ordinarily bifunctional) cross-linking agent. Methods for the manufacture of hapten immunogens are conventional per se, and any of the methods used heretofore for conjugating haptens to immunogenic polypeptides or the like are suitably employed here as well, taking into account the functional groups on the precursors or hydrolytic products which are available for cross-linking and the likelihood of producing antibodies specific to the epitope in question as opposed to the immunogenic substance.

Typically the polypeptide is conjugated to a site on the compound of the invention distant from the epitope to be recognized.

The conjugates are prepared in conventional fashion. For example, the cross-linking agents N-hydroxysuccinimide, succinic anhydride or alkN═C═Nalk are useful in preparing the conjugates of this invention. The conjugates comprise a compound of the invention attached by a bond or a linking group of 1-100, typically, 1-25, more typically 1-10 carbon atoms to the immunogenic substance. The conjugates are separated from starting materials and by products using chromatography or the like, and then are sterile filtered and vialed for storage.

The compounds of this invention are cross-linked for example through any one or more of the following groups: a hydroxyl group of U₁; a carboxyl group of E₁; a carbon atom of U₁, E₁, G₁, or T₁, in substitution of H; and an amine group of G₁. Included within such compounds are amides of polypeptides where the polypeptide serves as an above-described R_{6c} or R_{6b} groups.

Animals are typically immunized against the immunogenic conjugates or derivatives and antisera or monoclonal antibodies prepared in conventional fashion.

The compounds of the invention are useful for maintaining the structural integrity of glycoproteins in recombinant cell culture, i.e., they are added to fermentations in which glycoproteins are being produced for recovery so as to inhibit neuraminidase-catalyzed cleavage of the desired glycoproteins. This is of particular value in the recombinant synthesis of proteins in heterologous host cells that may disadvantageously degrade the carbohydrate portion of the protein being synthesized.

The compounds of the invention are polyfunctional. As such they represent a unique class of monomers for the synthesis of polymers. By way of example and not limitation, the polymers prepared from the compounds of this invention include polyamides and polyesters.

The present compounds are used as monomers to provide access to polymers having unique pendent functionalities. The compounds of this invention are useful in homopolymers, or as comonomers with monomers which do not fall within the scope of the invention. Homopolymers of the compounds of this invention will have utility as cation exchange agents (polyesters or polyamides) in the preparation of molecular sieves (polyamides), textiles, fibers, films, formed articles and the like where the acid functionality E₁ is esterified to a hydroxyl group in U₁, for example, whereby the pendant basic group G₁ is capable of binding acidic functionalities such as are found in polypeptides whose purification is desired. Polyamides are prepared by cross-linking E₁ and G₁, with U₁ and the adjacent portion of the ring remaining free to function as a hydrophilic or hydrophobic affinity group, depending up the selection of the U₁ group. The preparation of these polymers from the compounds of the invention is conventional per se.

The compounds of the invention are also useful as a unique class of polyfunctional surfactants. Particularly when U₁ does not contain a hydrophilic substituent and is, for example, alkyl or alkoxy, the compounds have the properties of bi-functional surfactants. As such they have useful surfactant, surface coating, emulsion modifying, rheology modifying and surface wetting properties.

As polyfunctional compounds with defined geometry and carrying simultaneously polar and non-polar moieties, the compounds of the invention are useful as a unique class of phase transfer agents. By way of example and not limitation, the compounds of the invention are useful in phase transfer catalysis and liquid/liquid ion extraction (LIX).

The compounds of the invention optionally contain asymmetric carbon atoms in groups U₁, E₁, G₁, and T₁. As such, they are a unique class of chiral auxiliaries for use in the synthesis or resolution of other optically active materials. For example, a racemic mixture of carboxylic acids can be resolved into its component enantiomers by: 1) forming a mixture of diastereomeric esters or amides with a compound of the invention wherein U₁ is an asymmetric hydroxyalkane or amino alkane group; 2) separating the diastereomers; and 3) hydrolyzing the ester structure. Racemic alcohols are separated by ester formation with an acid group of E₁. Further, such a method can be used to resolve the compounds of the invention themselves if optically active acids or alcohols are used instead of racemic starting materials.

The compounds of this invention are useful as linkers or spacers in preparing affinity absorption matrices, immobilized enzymes for process control, or immunoassay reagents. The compounds herein contain a multiplicity of functional groups that are suitable as sites for cross-linking desired substances. For example, it is conventional to link affinity reagents such as hormones, peptides, antibodies, drugs, and the like to insoluble substrates. These insolublized reagents are employed in known fashion to absorb binding partners for the affinity reagents from manufactured preparations, diagnostic samples and other impure mixtures. Similarly, immobilized enzymes are used to perform catalytic conversions with facile recovery of enzyme. Bifunctional compounds are commonly used to link analytes to detectable groups in preparing diagnostic reagents.

Many functional groups in the compounds of this invention are suitable for use in cross-linking. For example, the carboxylic or phosphonic acid of group E₁ is used to form esters with alcohols or amides with amines of the reagent to be cross-linked. The G₁ sites substituted with OH, NHR₁, SH, azido (which is reduced to amino if desired before cross-linking), CN, NO₂, amino, guanidino, halo and the like are suitable sites. Suitable protection of reactive groups will be used where necessary while assembling the cross-linked reagent to prevent polymerization of the bifunctional compound of this invention. In general, the compounds here are used by linking them through carboxylic or phosphonic acid to the hydroxyl or amino groups of the first linked partner, then covalently bonded to the other binding partner through a T₁ or G₁ group. For example a first binding partner such as a steroid hormone is esterified to the carboxylic acid of a compound of this invention and then this conjugate is cross-linked through a G₁ hydroxyl to cyanogen bromide activated Sepaharose, whereby immobilized steroid is obtained. Other chemistries for conjugation are well known. See for example Maggio, “Enzyme-Immunoassay” (CRC, 1988, pp 71-135) and references cited therein.

As noted above, the therapeutically useful compounds of this invention in which the W₁, or G₁ carboxyl, hydroxyl or amino groups are protected are useful as oral or sustained release forms. In these uses the protecting group is removed in vivo, e.g., hydrolyzed or oxidized, so as to yield the free carboxyl, amino or hydroxyl. Suitable esters or amides for this utility are selected based on the substrate specificity of esterases and/or carboxypeptidases expected to be found within cells where precursor hydrolysis is desired. To the extent that the specificity of these enzymes is unknown, one will screen a plurality of the compounds of this invention until the desired substrate specificity is found. This will be apparent from the appearance of free compound or of antiviral activity. One generally selects amides or esters of the invention compound that are (i) not hydrolyzed or hydrolyzed comparatively slowly in the upper gut, (ii) gut and cell permeable and (iii) hydrolyzed in the cell cytoplasm and/or systemic circulation. Screening assays preferably use cells from particular tissues that are susceptible to influenza infection, e.g. the mucous membranes of the bronchopulmonary tract. Assays known in the art are suitable for determining in vivo bioavailability including intestinal lumen stability, cell permeation, liver homogenate stability and plasma stability assays. However, even if the ester, amide or other protected derivatives are not converted in vivo to the free carboxyl, amino or hydroxyl groups, they remain useful as chemical intermediates.

### Exemplary Methods of Making the Compounds of the Invention

The invention also relates to methods of making the compositions of the invention. The compositions are prepared by any of the applicable techniques of organic synthesis. Many such techniques are well known in the art. However, many of the known techniques are elaborated in “Compendium of Organic Synthetic Methods” (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, jr., 1980; Vol. 5, Leroy G. Wade, Jr., 1984; and Vol. 6, Michael B. Smith; as well as March, J., “Advanced Organic Chemistry, Third Edition”, (John Wiley & Sons, New York, 1985), “Comprehensive Organic Synthesis. Selectivity, Strategy & Efficiency in Modern Organic Chemistry. In 9 Volumes”, Barry M. Trost, Editor-in-Chief (Pergamon Press, New York, 1993 printing).

A number of exemplary methods for the preparation of the compositions of the invention are provided below. These methods are intended to illustrate the nature of such preparations are not intended to limit the scope of applicable methods.

Generally, the reaction conditions such as temperature, reaction time, solvents, workup procedures, and the like, will be those common in the art for the particular reaction to be performed. The cited reference material, together with material cited therein, contains detailed descriptions of such conditions. Typically the temperatures will be −100° C. to 200° C., solvents will be aprotic or protic, and reaction times will be 10 seconds to 10 days. Workup typically consists of quenching any unreacted reagents followed by partition between a water/organic layer system (extraction) and separating the layer containing the product.

Oxidation and reduction reactions are typically carried out at temperatures near room temperature (about 20° C.), although for metal hydride reductions frequently the temperature is reduced to 0° C. to −100° C., solvents are typically aprotic for reductions and may be either protic or aprotic for oxidations. Reaction times are adjusted to achieve desired conversions.

Condensation reactions are typically carried out at temperatures near room temperature, although for non-equilibrating, kinetically controlled condensations reduced temperatures (0° C. to −100° C.) are also common. Solvents can be either protic (common in equilibrating reactions) or aprotic (common in kinetically controlled reactions).

Standard synthetic techniques such as azeotropic removal of reaction by-products and use of anhydrous reaction conditions (e.g. inert gas environments) are common in the art and will be applied when applicable.

One exemplary method of preparing the compounds of the invention is shown in Scheme 1 below. A detailed description of the methods is found in the Experimental section below.

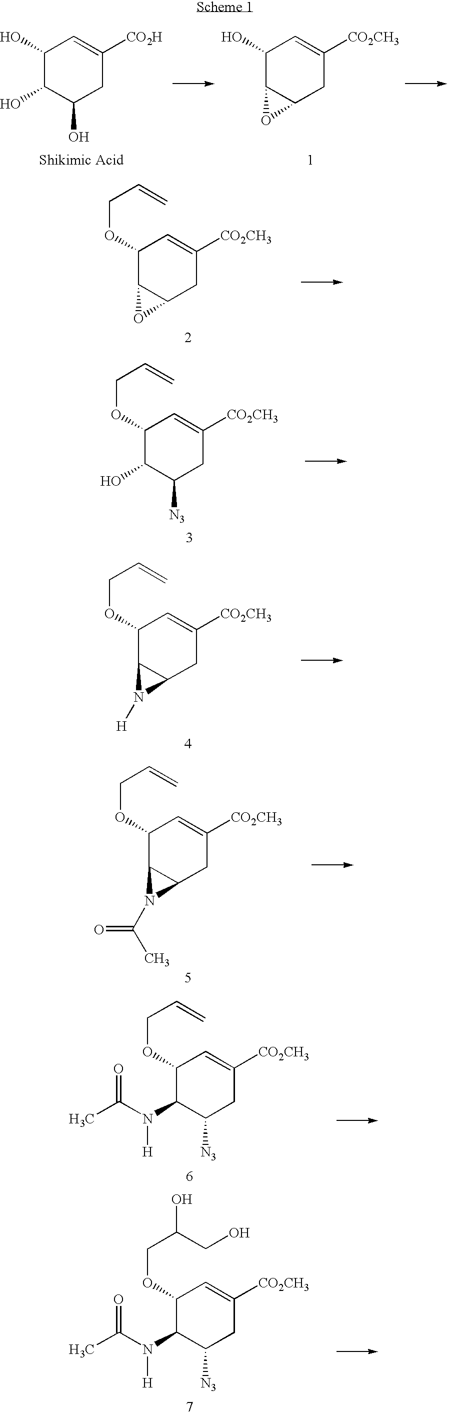

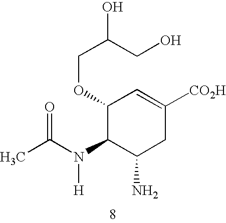

Modifications of Scheme 1 to form additional embodiments is shown in Schemes 2-4.

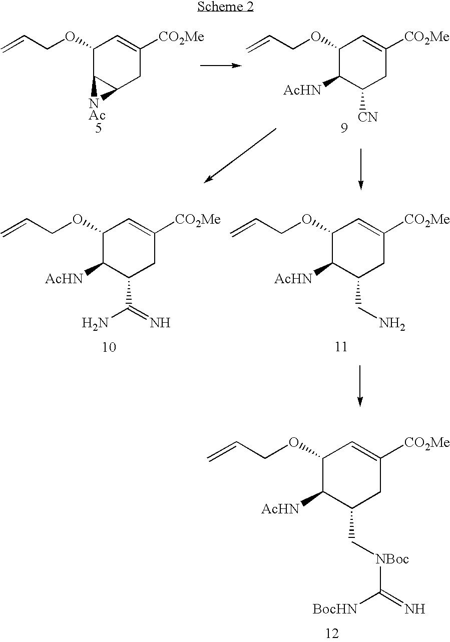

Scheme 2

Aziridine 5 is converted to the amino nitrile 9 by Yb(CN)₃ catalyzed addition of TMSCN according to the procedure of Utimoto and co-workers, “Tetrahedron Lett.”, 31:6379 (1990).

Conversion of nitrile 9 to the corresponding amidine 10 is accomplished using a standard three step sequence: i) H₂S; ii) CH₃I; iii) NH₄OAc. A typical conversion is found in “J. Med. Chem.”, 36:1811 (1993). Nitrile 9 is converted to the amino methyl compound 11 by reduction using any of the available methods found in “Modern Synthetic Reactions” 2nd ed. H. O. House, Benjamin/Cummings Publishing Co., 1972.

Amino methyl compound 11 is converted to the bis-Boc protected guanidino compound 12 by treating 11 with N,N′-bis-Boc-1H-pyrazole-1-carboxamidine according to the method found in “Tetrahedron Lett.”, 36:299 (1995).

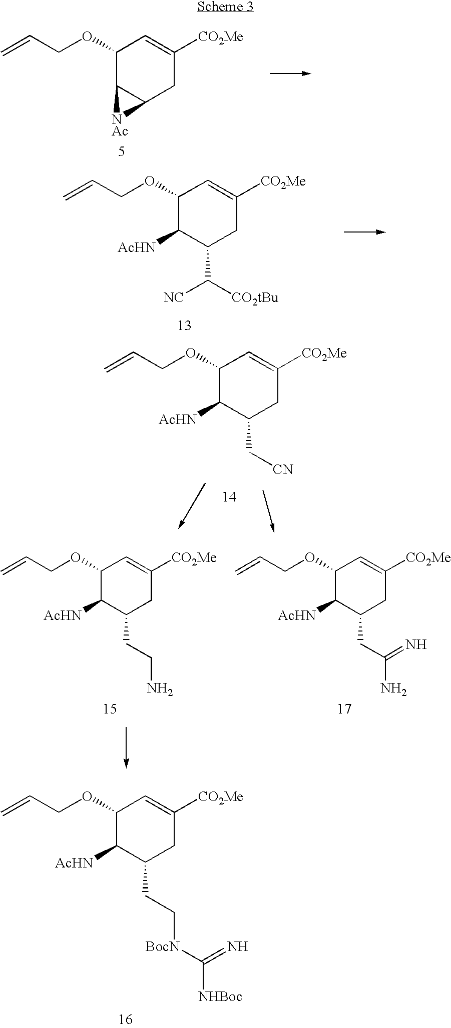

Scheme 3

The aziridine 5 is opened with α-cyano acetic acid t-butyl ester to give 13. Aziridine openings of this type are found in “Tetrahedron Lett.”, 23:5021 (1982). Selective hydrolysis of the t-butyl ester moiety under acidic condtions followed by decarboxylation gives nitrile 14.

Reduction of 14 to the amino ethyl derivative 15 is accomplished in the same fashion as the conversion of 9 to 11. The amine 15 is then converted into the guanidino derivative 16 with N,N′-bis-Boc-1H-pyrazole-1-carboxamidine according to the method found in “Tetrahedron Lett.”, 36:299 (1995).

The nitrile 14 is converted to the corresponding amidine 17 using the same sequence described above for the conversion of 9 to 10.

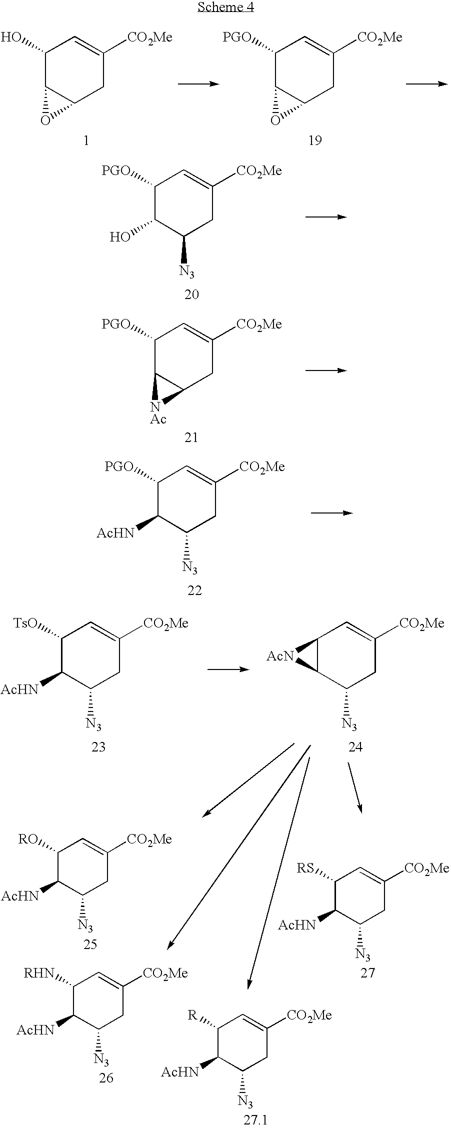

Scheme 4

The epoxy alcohol 1 is protected (PG=protecting group), for example with MOMCl. Typical conditions are found in “Protective Groups in Organic Synthesis” 2nd ed., T. W. Greene and P. G. M. Wuts, John Wiley & Sons, New York, N.Y., 1991.

The epoxide 19 is opened with NaN₃/NH₄Cl to the amino alcohol 20 according to the procedure of Sharpless and co-workers, “J. Org. Chem.”, 50:1557 (1985).

Reduction of 20 to the N-acetyl aziridine 21 is accomplished in a three step sequence: 1) MsCl/triethyl amine; 2) H₂/Pd; 3) AcCl/pyridine. Such transformations can be found in “Angew. Chem. Int. Ed. Engl.”, 33:599 (1994).

Aziridine 21 is converted to the azido amide 22 by opening with NaN₃/NH₄Cl in DMF at 65° C. as described in “J. Chem. Soc. Perkin Trans I”, 801 (1976).

Removal of the MOM protecting group of 22 is accomplished using the methods described in “Protective Groups in Organic Synthesis” 2nd ed., T. W. Greene and P. G. M. Wuts, John Wiley & Sons, New York, N.Y., 1991. The resulting alcohol is converted directly to aziridine 24 with TsCl in pyridine. Such transformations are found in “Angew. Chem. Int. Ed. Engl.”, 33:599 (1994).

Aziridine 24 is then reacted with ROH, RNH₂, RSH or an organometallic (metal-R) to give the corresponding ring opened derivatives 25, 26, 27 and 27.1 respectively. Aziridine openings of this type are found in “Tetrahedron Lett.”, 23:5021 (1982) and “Angew. Chem. Int. Ed. Engl.”, 33:599 (1994).

Scheme 5

Another class of compounds of the invention are prepared by the method of Schemes 5a and 5b. Quinic acid is converted to 28 by the method of Shing, T. K. M.; et al.; “Tetrahedron”, 47(26):4571 (1991). Mesylation with MsCl in TEA/CH₂Cl₂ will give 29 which is reacted with NaN₃ in DMF to give 30. Reaction of 30 with TFA in CH₂Cl₂ will give 31 which is mesylated with MsCl in TEA/CH₂Cl₂ to give 32. Reaction with triphenylphosphine in water will give 33 which is converted to 35 by sequential application of: 1) CH₃C(O)Cl in pyridine, 2) NaN₃ in DMF, and 3) NaH in THF. Alkylation of 35 with a wide variety of nucleophiles common in the art will provide a number of compounds such as 36. Methods for elaboration of the compounds such as 36 to other embodiments of the invention will be similar to those described above.

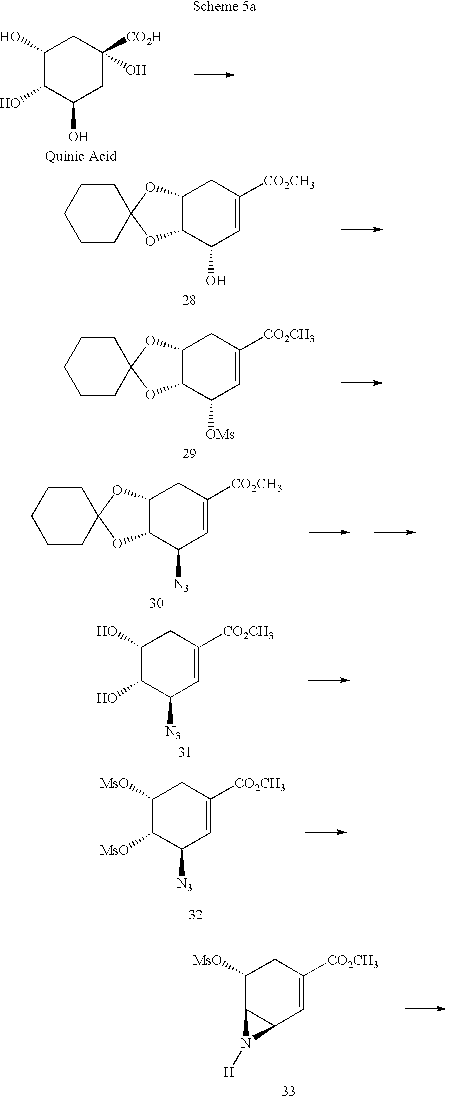

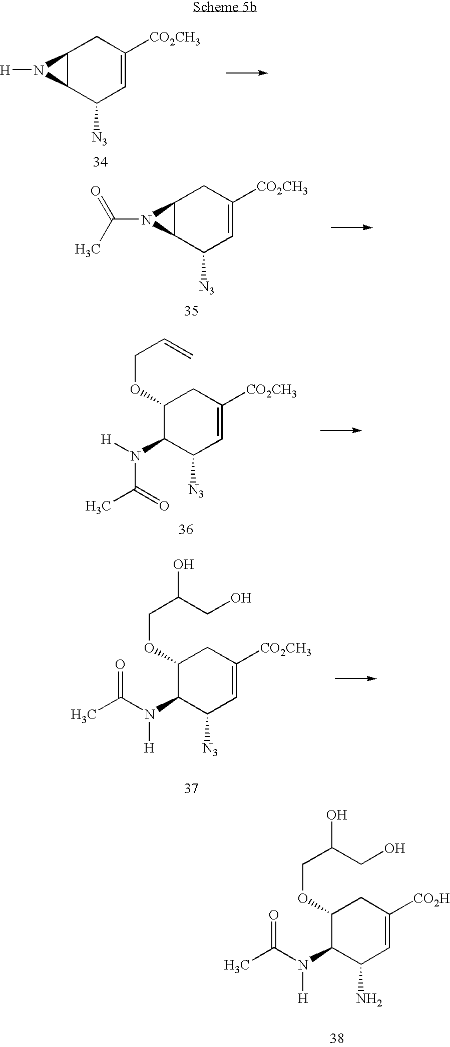

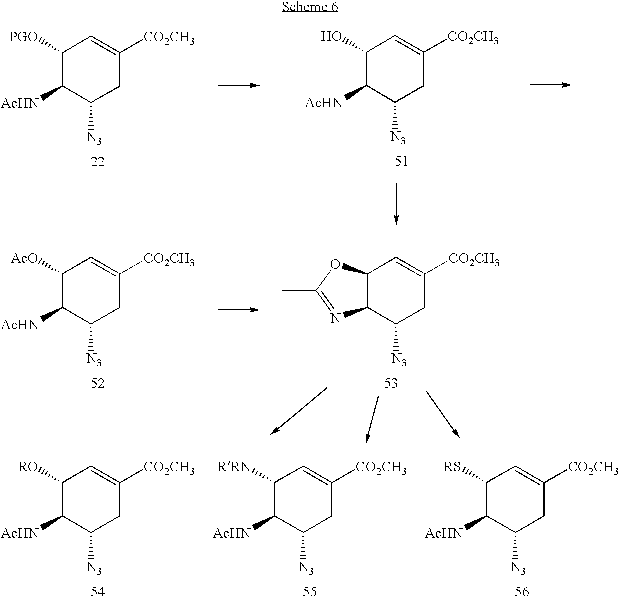

Scheme 6

Another class of compounds of the invention are prepared by the method of Scheme 6. Protected alcohol 22 (PG=methoxymethyl ether) is deprotected under standard conditions described in “Protective Groups in Organic Synthesis” 2nd ed., T. W. Greene and P. G. M. Wuts, John Wiley & Sons, New York, N.Y., 1991. Alcohol 51 is converted to acetate 52 with acetic anhydride and pyridine under standard conditions. Acetate 52 is treated with TMSOTf or BF3.OEt to afford oxazoline 53. Such transformations are described in “Liebigs Ann. Chem.”, 129 (1991) and “Carbohydrate Research”, 181 (1993), respectively. Alternatively, alcohol 51 is transformed to oxazoline 53 by conversion to the corresponding mesylate or tosylate 23 and subsequently cyclized to the oxazoline under standard conditions, as described in “J. Org. Chem.”, 50:1126 (1985) and “J. Chem. Soc.”, 1385 (1970). Oxazoline 53 is reacted with ROH, RR′NH, or RSH (wherein R and R′ are selected to be consistent with the definition of W₆ above) provide the corresponding ring opened derivatives 54, 55, and 56 respectively. Such transformations are described in “J. Org. Chem.”, 49:4889 (1984) and “Chem. Rev.”, 71:483 (1971).

Schemes 7-63

Other exemplary methods of preparing the compounds of the invention are shown in Schemes 7-63 below. A detailed description of the methods is found in the Experimental section below.

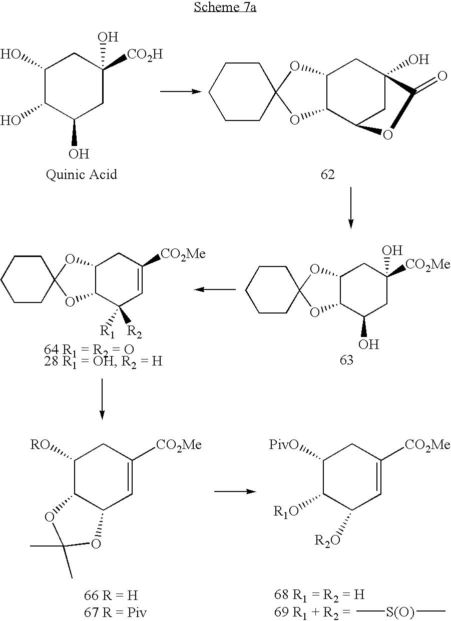

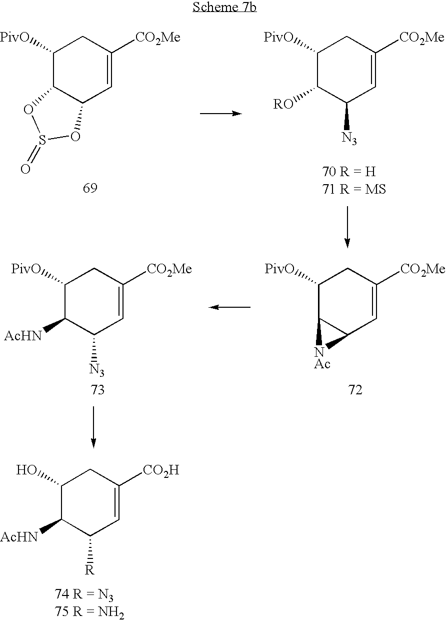

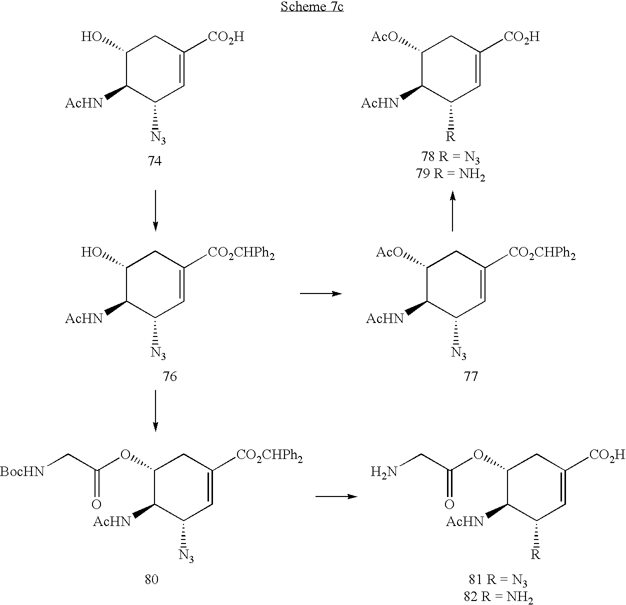

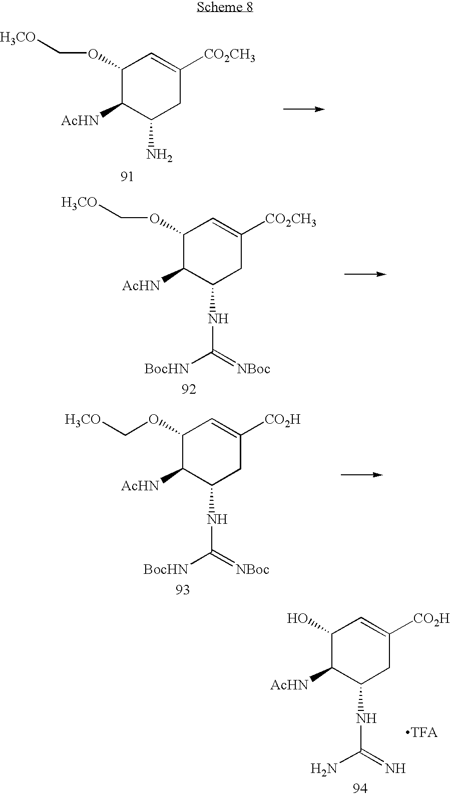

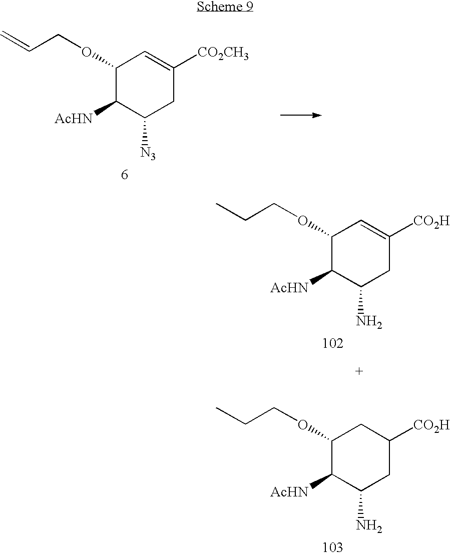

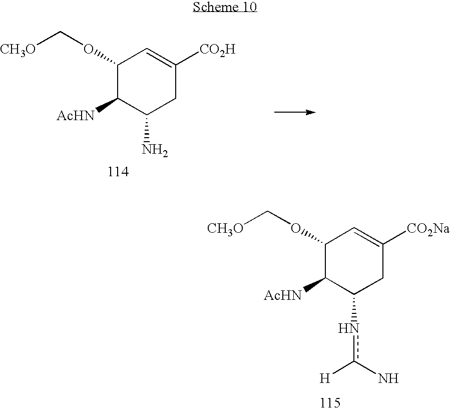

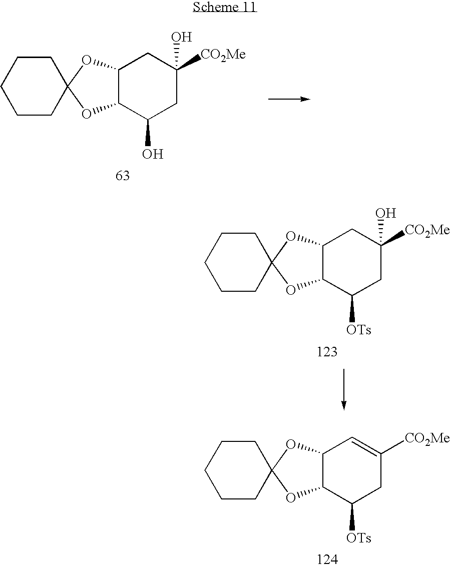

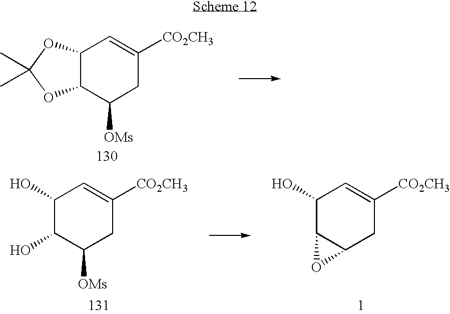

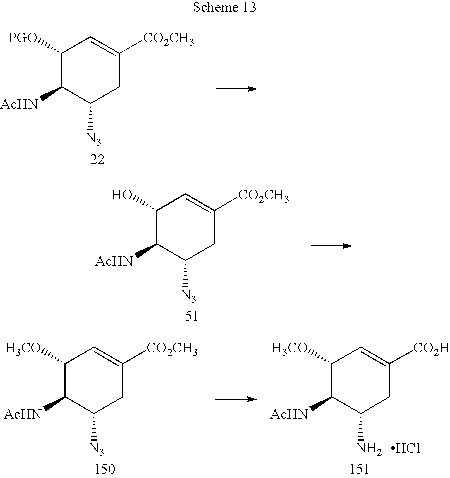

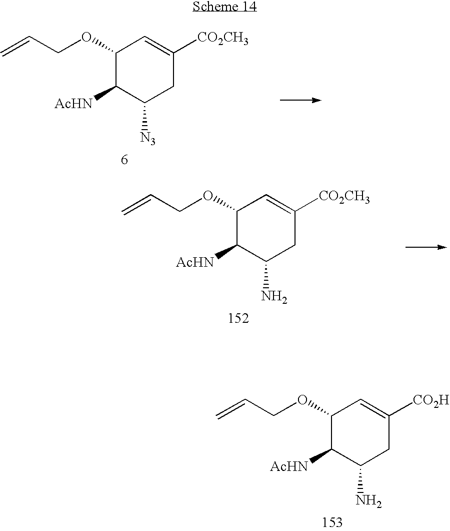

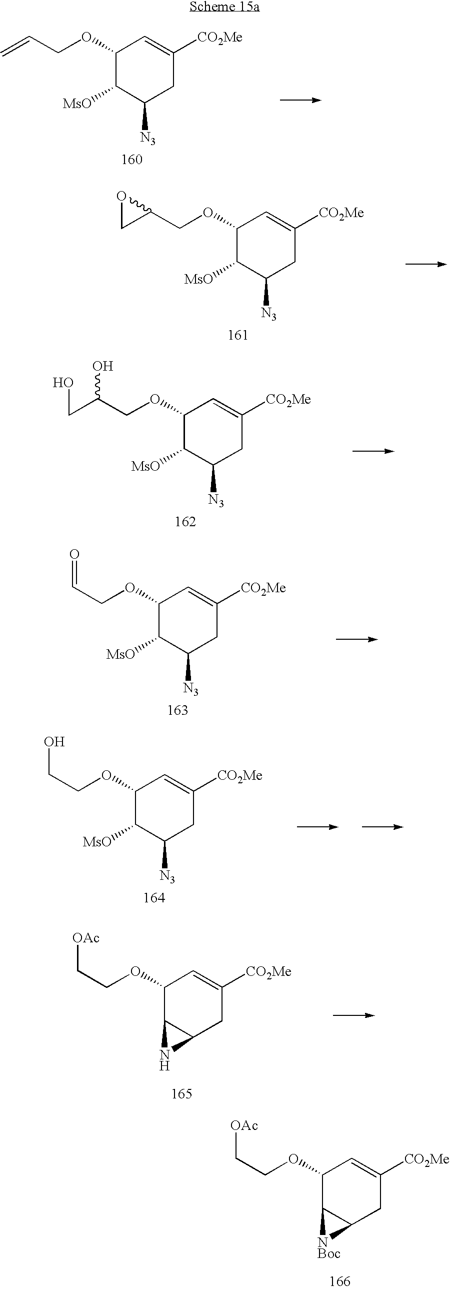

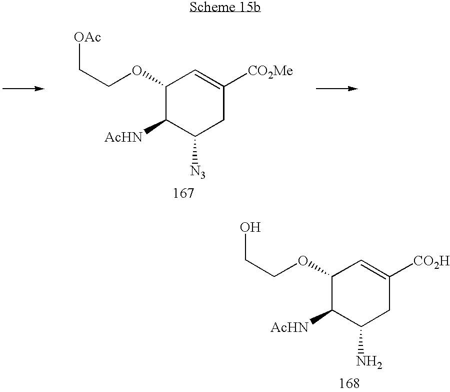

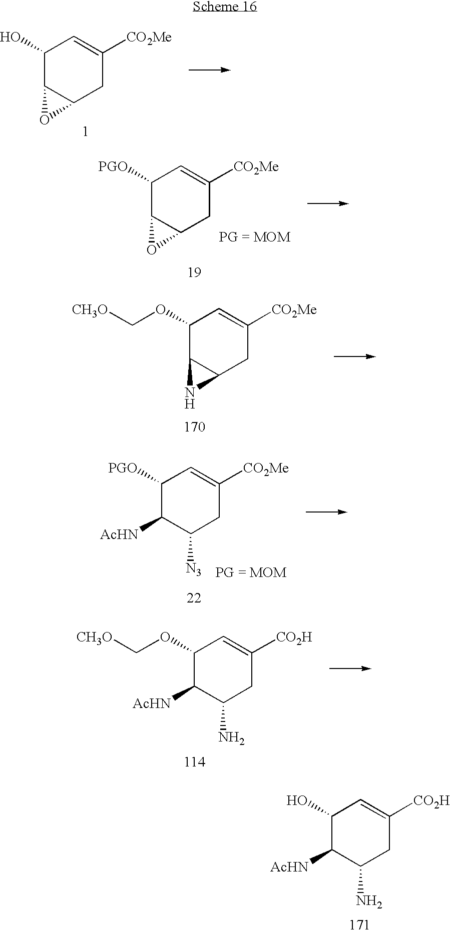

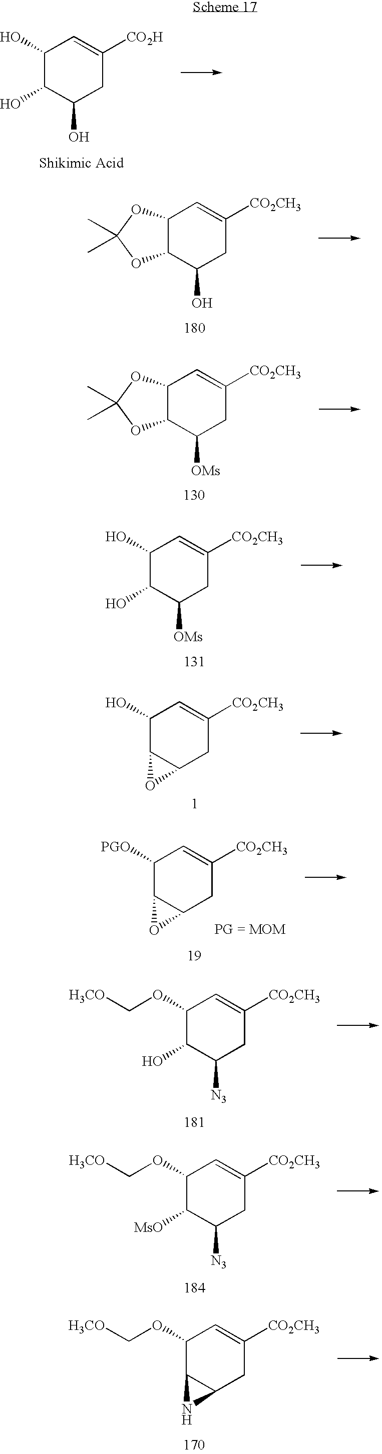

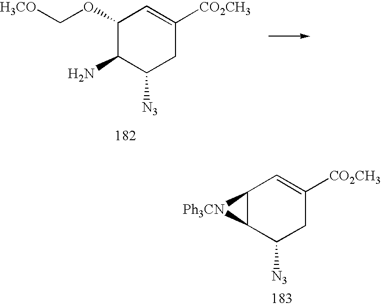

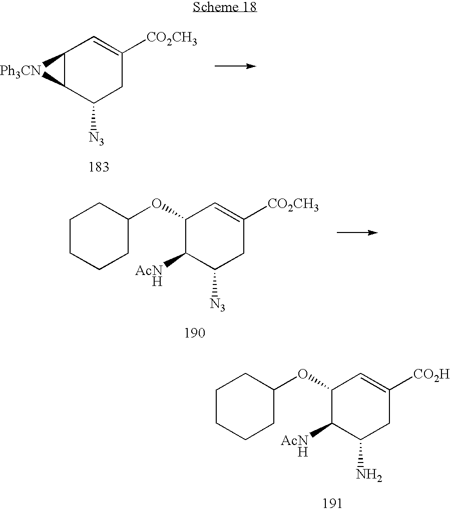

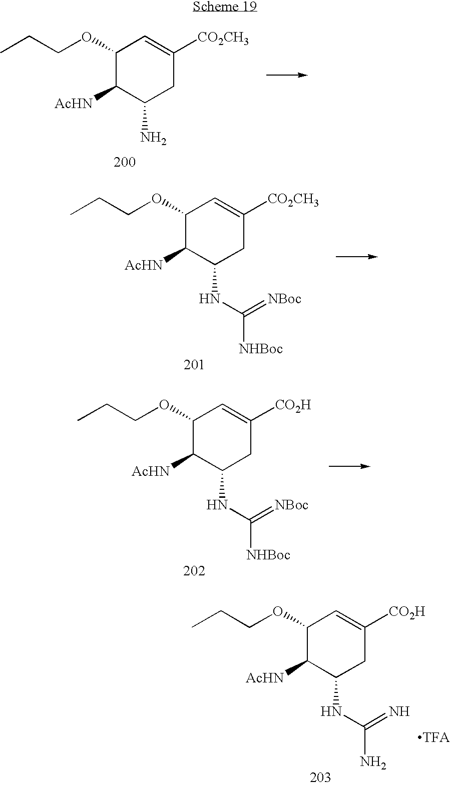

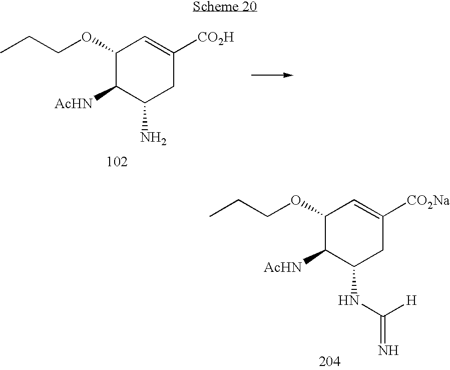

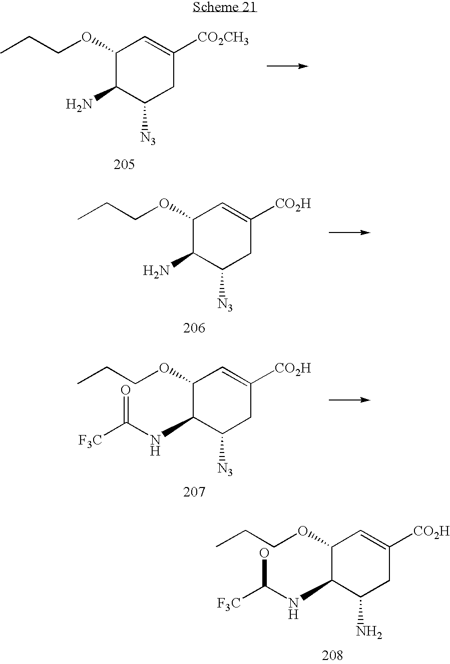

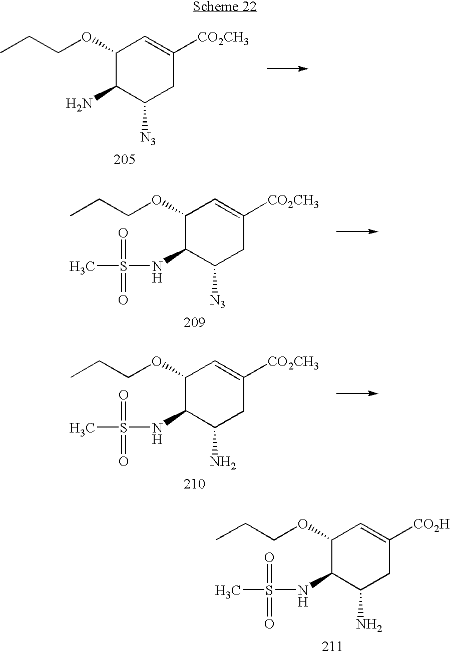

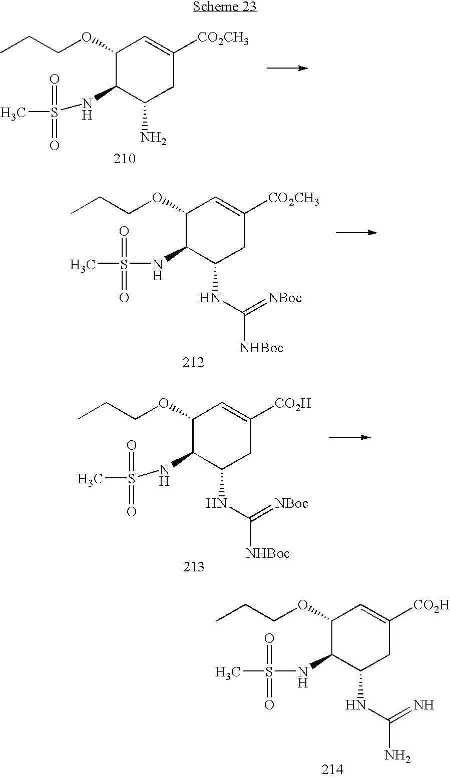

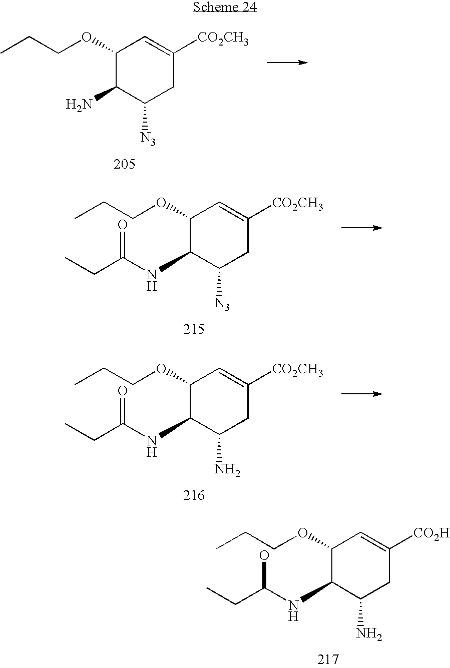

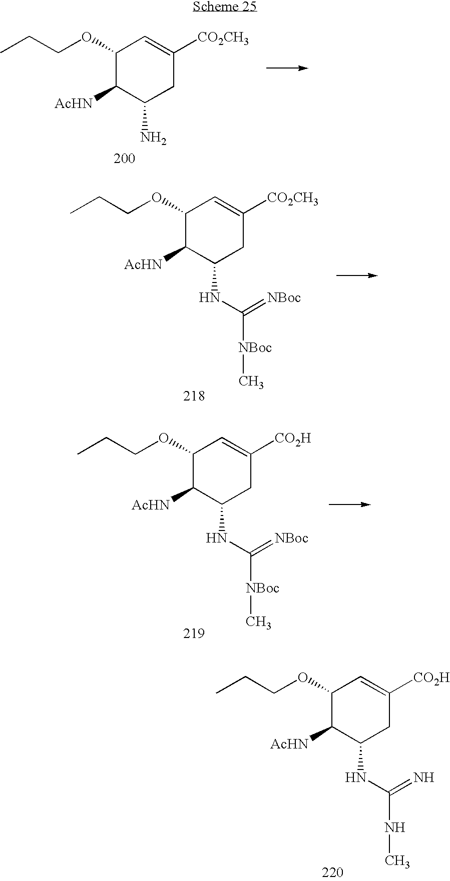

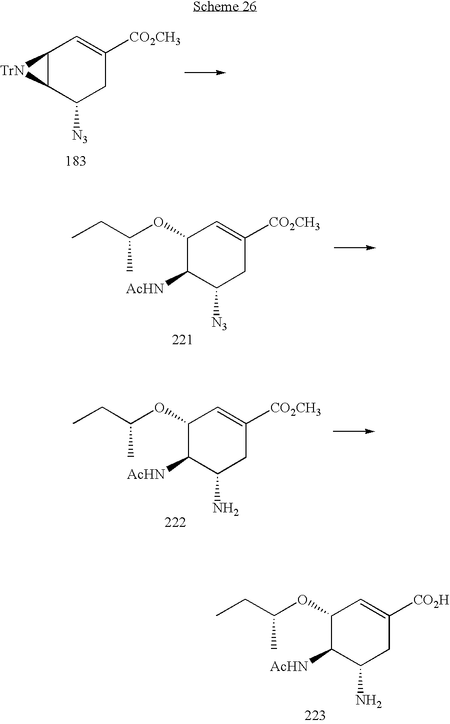

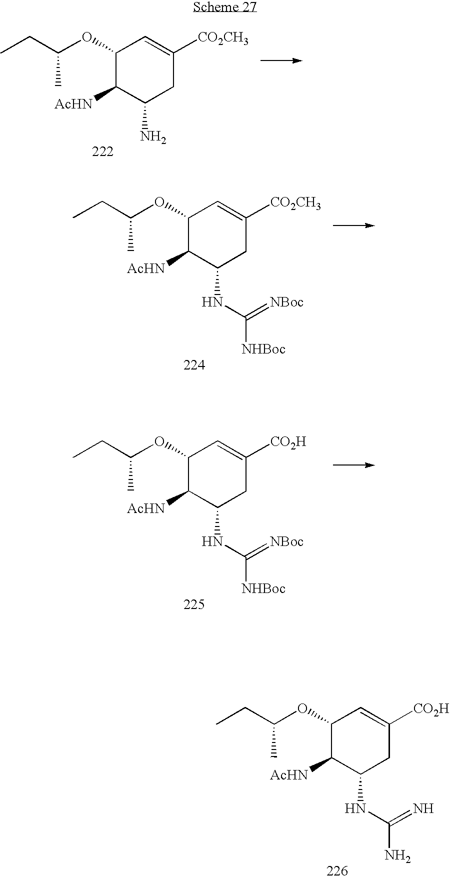

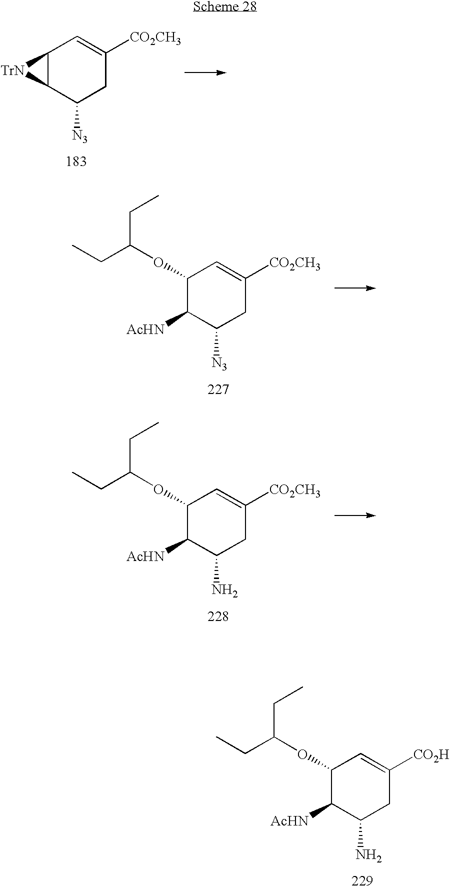

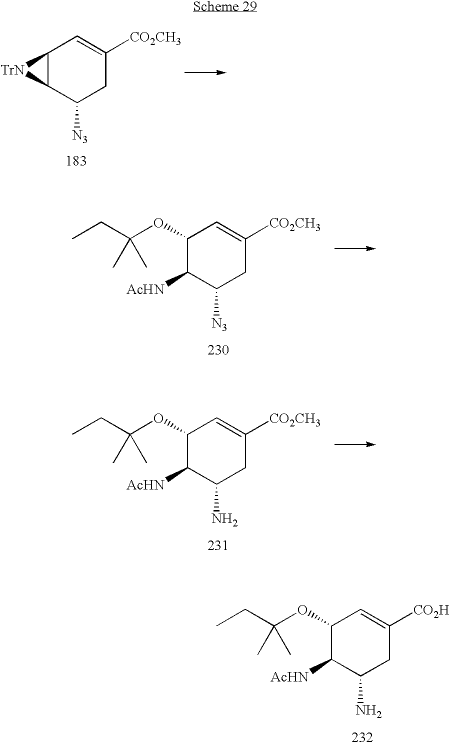

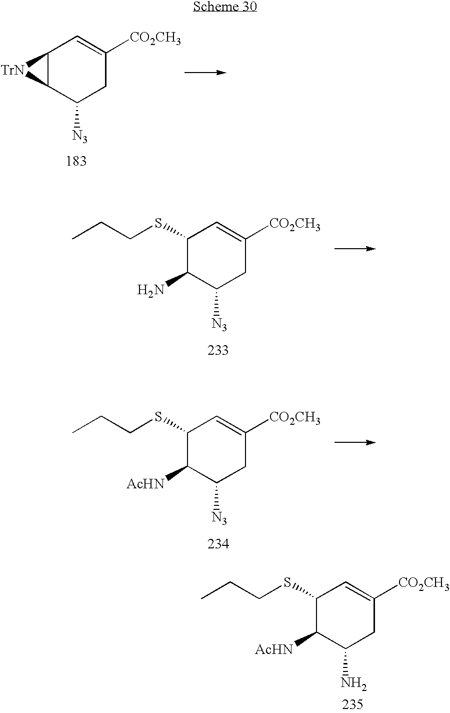

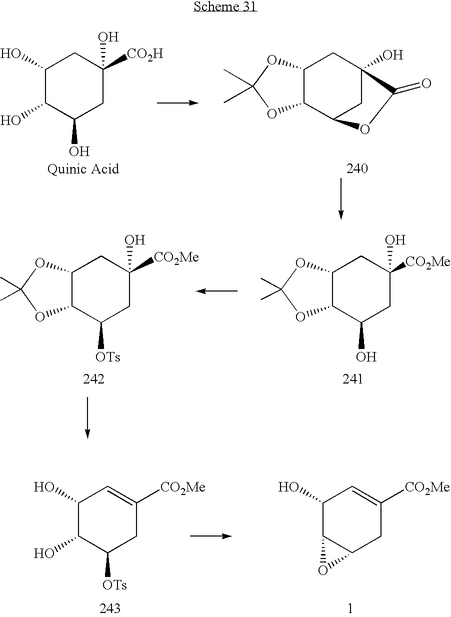

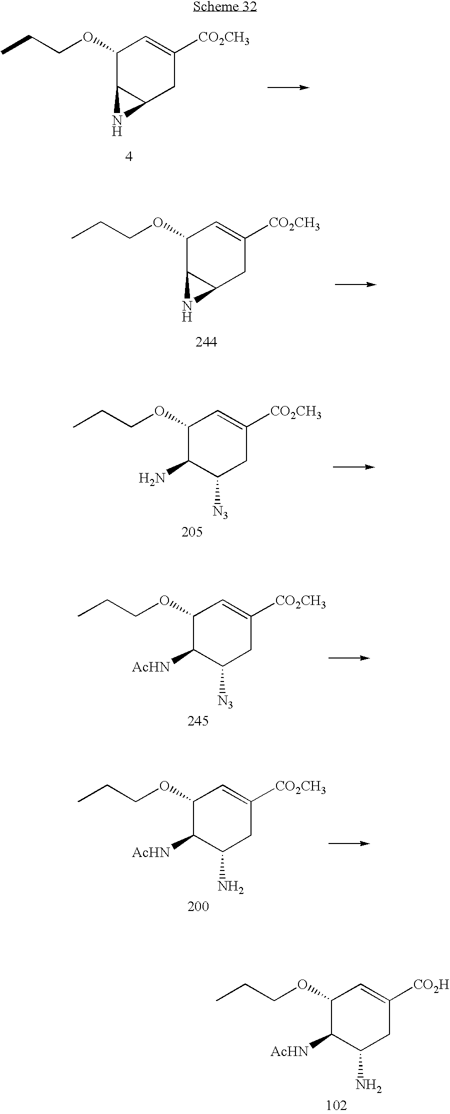

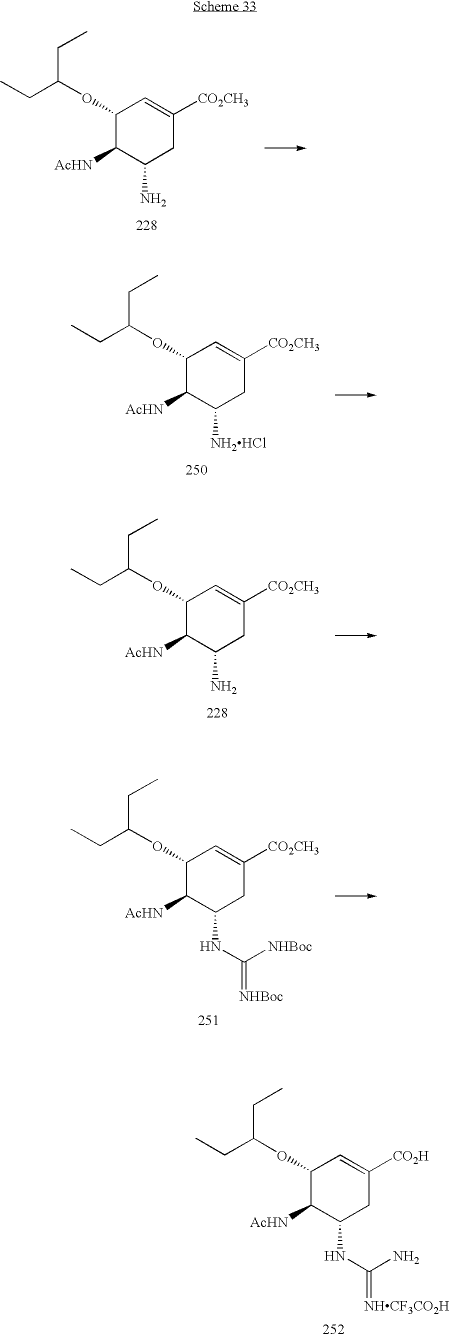

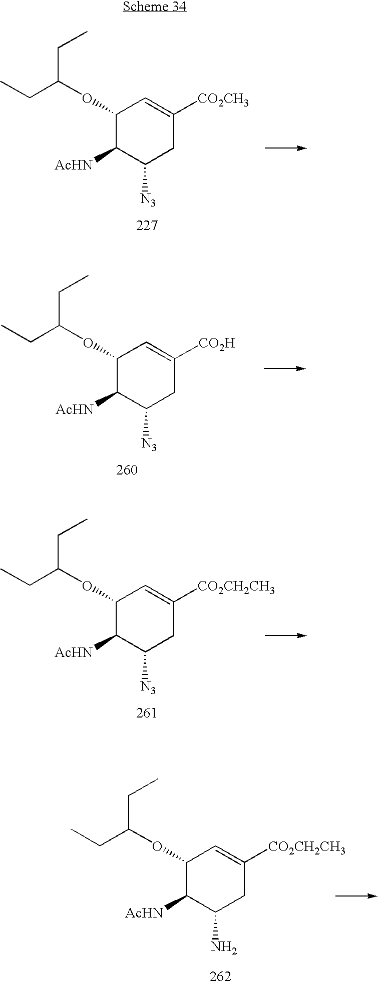

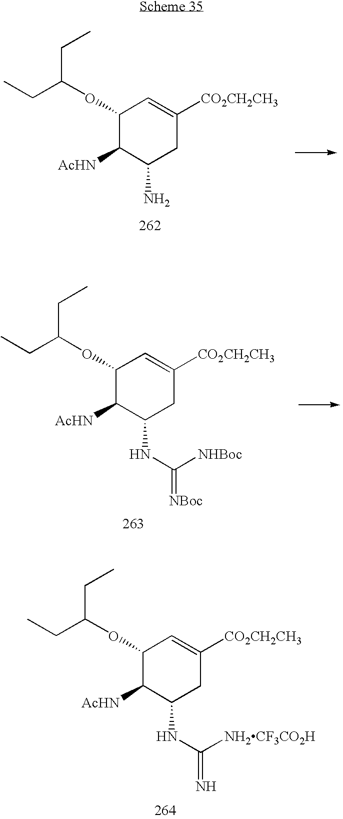

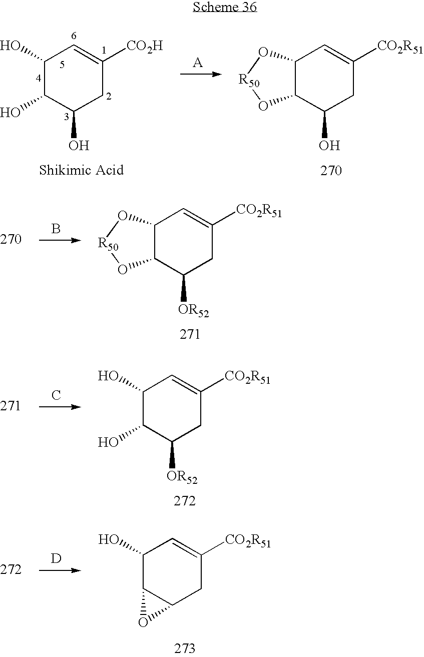

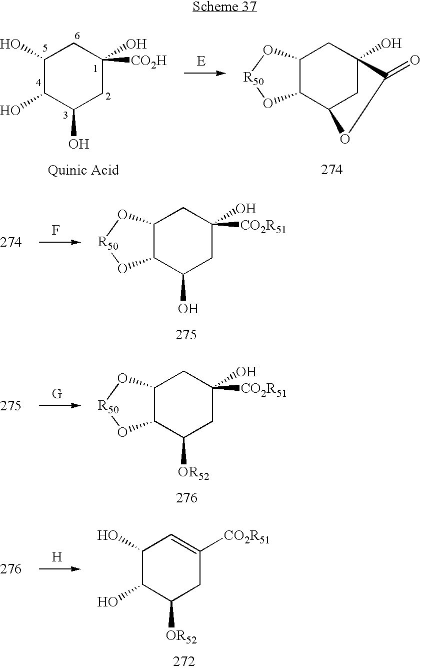

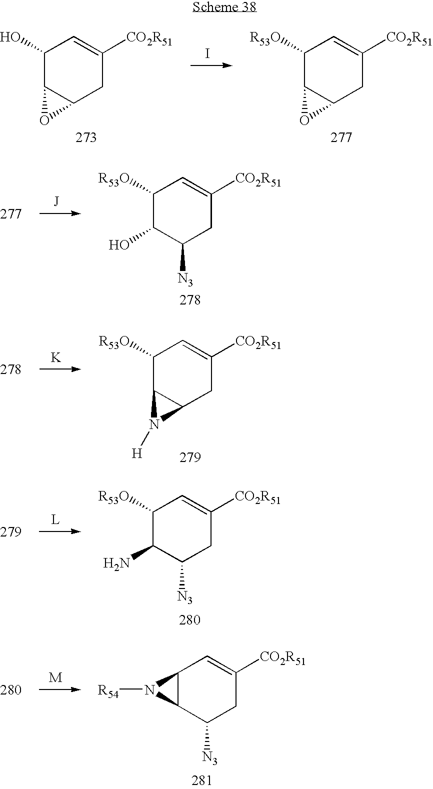

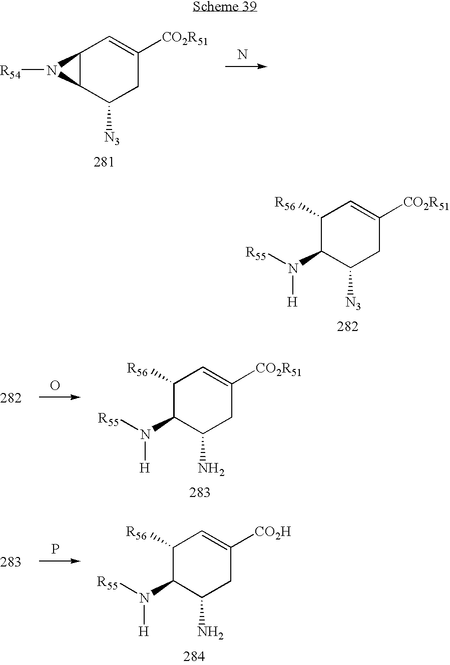

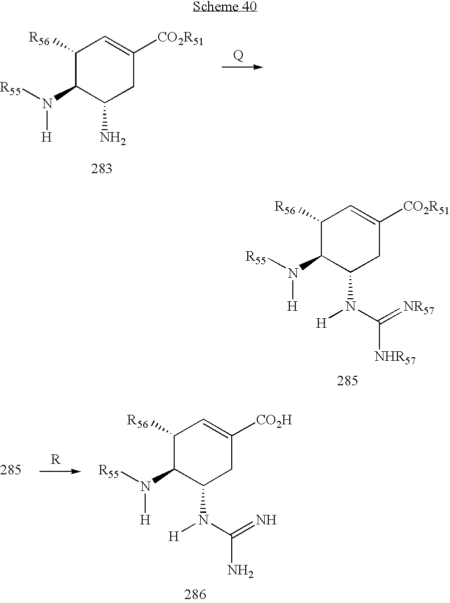

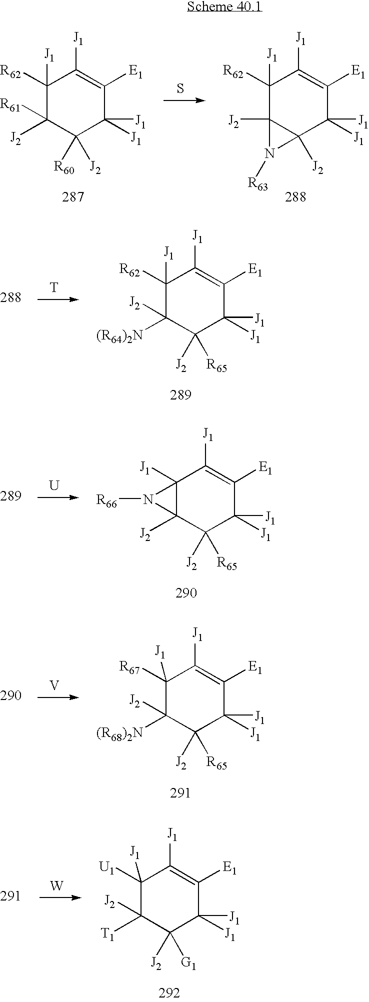

Additional embodiments of methods of making and using compositions of the invention are depicted in Schemes 36-40.1. One aspect of the invention is directed to methods of making compounds of the invention comprising processes A, B, C, D, E, F, G, H, I, J, K, L, M, N, O, P, Q, R, S, T, U, V or W of Schemes 36-40.1, alone or in combination with each other. Table 27 describes exemplary method embodiments of processes A-W. Each embodiment is an individual method using the unit processes A-W alone or in combination. Each method embodiment of Table 27 is separated by a “;”. If the embodiment is a single letter than it corresponds to one of the processes A-W. If it is more than one letter than it corresponds to each of the processes performed sequentially in the order indicated.

Other aspects of the invention are directed to methods of using shikimic acid to prepare compound 270 shown as A in Scheme 36, methods of using compound 270 to prepare compound 271 shown as B in Scheme 36, methods of using compound 271 to prepare compound 272 shown as C in Scheme 36, methods of using compound 272 to prepare compound 273 shown as D in Scheme 36, methods of using quinic acid to prepare compound 274 shown as E in Scheme 37, methods of using compound 274 to prepare compound 275 shown as F in Scheme 37, methods of using compound 275 to prepare compound 276 shown as G in Scheme 37, methods of using compound 276 to prepare compound 272 shown as H in Scheme 37, methods of using compound 273 to prepare compound 277 shown as I in Scheme 38, methods of using compound 277 to prepare compound 278 shown as J in Scheme 38, methods of using compound 278 to prepare compound 279 shown as K in Scheme 38, methods of using compound 279 to prepare compound 280 shown as L in Scheme 38, methods of using compound 280 to prepare compound 281 shown as M in Scheme 38, methods of using compound 281 to prepare compound 282 shown as N in Scheme 39, methods of using compound 282 to prepare compound 283 shown as O in Scheme 39, methods of using compound 283 to prepare compound 284 shown as P in Scheme 39, methods of using compound 283 to prepare compound 285 shown as O in Scheme 40, methods of using compound 285 to prepare compound 286 shown as R in Scheme 40, methods of using compound 287 to prepare compound 288 shown as S in Scheme 40.1, methods of using compound 288 to prepare compound 289 shown as T in Scheme 40.1, methods of using compound 289 to prepare compound 290 shown as U in Scheme 40.1, methods of using compound 290 to prepare compound 291 shown as V in Scheme 40.1, and methods of using compound 291 to prepare compound 292 shown as W in Scheme 40.1.

General aspects of these exemplary methods are described below and in the Examples. Each of the products of the following processes is optionally separated, isolated, and/or purified prior to its use in subsecquent processes.

The terms “treated”, “treating”, “treatment”, and the like, mean contacting, mixing, reacting, allowing to react, bringing into contact, and other terms common in the art for indicating that one or more chemical entities is treated in such a manner as to convert it to one or more other chemical entities. This means that “treating compound one with compound two” is synonymous with “allowing compound one to react with compound two”, “contacting compound one with compound two”, “reacting compound one with compound two”, and other expressions common in the art of organic synthesis for reasonably indicating that compound one was “treated”, “reacted”, “allowed to react”, etc., with compound two.

“Treating” indicates the reasonable and usual manner in which organic chemicals are allowed to react. Normal concentrations (0.01M to 10M, typically 0.1M to 1M), temperatures (−100° C. to 250° C., typically −78° C. to 150° C.,more typically −78° C. to 100° C., still more typically 0° C. to 100° C.), reaction vessels (typically glass, plastic, metal), solvents, pressures, atmospheres (typically air for oxygen and water insensitive reactions or nitrogen or argon for oxygen or water sensitive), etc., are intended unless otherwise indicated. The knowledge of similar reactions known in the art of organic synthesis are used in selecting the conditions and apparatus for “treating” in a given process. In particular, one of ordinary skill in the art of organic sysnthesis selects conditions and apparatus reasonably expected to successfully carry out the chemical reactions of the described processes based on the knowledge in the art.

Process A, Scheme 36

Shikimic acid is used to prepare compound 270 by the following process.

The cis-4,5-diol function of shikimic acid is differentiated from the carboxylic acid at carbon 1 by selective protection of these two functionalities. Typically the cis-4,5-diol function is protected as a cyclic ketal and the carboxylic acid function is protected as an ester.

R₅₀ is an acid labile 1,2-diol protecting group such as those described in the above cited work of Greene, typically a cyclic ketal or acetal, more typically, a ketal of cyclohexanone or acetone. R₅₁ is an acid stable carboxylic acid protecting group such as those described in the above cited work of Greene, typically a linear, branched or cyclic alkyl, alkenyl, or alkynyl of 1 to 12 carbon atoms such as those shown as groups 2-7, 9-10, 15, or 100-660 of Table 2, more typically a linear or branched alkyl of 1 to 8 carbon atoms such as those shown as groups 2-5, 9, or 100-358 of Table 2, still more typically a linear or branched alkyl of 1 to 6 carbon atoms such as those shown as groups 2-5, 9, or 100-141 of Table 2, more typically yet, R₅₁ is methyl, ethyl, n-propyl, i-propyl, n-butyl, sec-butyl, i-butyl, or t-butyl.

Shikimic acid is reacted to protect the carboxylic acid with group R₅₁ and the cis-4,5-diol with group R₅₀. Typically shikimic acid is treated with an alcohol, such as methanol, ethanol, n-propanol, or i-propanol, and an acid catalyst, such as a mineral acid or a sulfonic acid such as methane, benzene or toluene sulfonic acid, followed by a dialkyl ketal or acetal of a ketone or aldehyde, such as 2,2-dimethoxy-propane, or 1,1-dimethoxy-cyclohexane, in the presence of the corresponding ketone or aldehyde, such as acetone or cyclohexanone. Optionally, the product of the alcohol and acid catalyst treatment is separated, isolated and/or purified prior to treatment with dialkyl ketal or acetal. Alternatively shikimic acid is treated with CH₂N₂.

Typically, the process comprises treating shikimic acid with an alkanol and a sulfonic acid followed by treating with a geminal-dialkoxyalkane or geminal dialkoxycycloalkane and an alkanone or cycloalkanone to form compound 270. More typically, the process comprises treating shikimic acid with an alkanol and a sulfonic acid; evaporating excess alkanol to form a residue; treating the residue with a geminal-dialkoxyalkane or geminal-dialkoxycycloalkane and an alkanone or cycloalkanone to form compound 270. Still more typically, the process comprises treating shikimic acid with methanol and para-toluenesulfonic acid; evaporating excess methanol to form a residue; treating the residue with 2,2-dimethoxypropane and acetone to form compound 270.

An exemplary embodiment of this process is given as Example 55 below.

Process B, Scheme 36

Compound 270 is used to prepare compound 271 by the following process.

The hydroxy group at position 3 is activated, typically, activated toward displacement reactions, more typically, activated toward epoxide ring forming displacement with an alcohol at position 4.

R₅₂ is an alcohol activating group, typically, an activating group toward displacement reactions, more typically, an activating group toward epoxide ring forming displacement with an alcohol at position 4. Such groups include Docket No. 234.PC2 those typical in the art such as sulfonic acid esters, more typically, methane, benzene or toluene sulfonic acid esters. In one embodiment, R₅₂, taken together with O (i.e. —OR₅₂), is a leaving group such as those common in the art.

Typically the process comprises treating compound 270 with an acid halide to form compound 271. More typically, the process comprises treating compound 270 with a sulfonic acid halide in a suitable solvent to form compound 271. Still more typically, the process comprises treating compound 270 with a sulfonic acid halide in a suitable solvent such as an amine, optionally, in the presence of a cosolvent, such as a haloalkane, to form compound 271. More typically yet, the process comprises treating compound 270 with methane sulfonyl chloride in triethylamine/dichloromethane to form compound 271.

An exemplary embodiment of this process is given as Example 56 below.

Process C, Scheme 36

Compound 271 is used to prepare compound 272 by the following process.

The acid labile protecting group (R₅₀) for the hydroxy groups at positions 4 and 5 is removed. Typically, R₅₀ is removed without substaintially removing base labile carboxylic acid protecting groups (e.g. R₅₁) or hydroxy activating groups (e.g. R₅₂). Still more typically, R₅₀ is cleaved under acidic conditions.

Typically the process comprises treating compound 271 with a protic solvent, more typically, in the presence of an acid catalyst as described above. Still more typically, the process comprises treating compound 271 with an alkanol as described above and an acid catalyst as described above. More typically yet, the process comprises treating compound 271 with methanol and para-toluene sulfonic acid to produce compound 272.

An exemplary embodiment of this process is given as Example 57 below.

Process D Scheme 36

Compound 272 is used to prepare compound 273 by the following process.

The activated hydroxy group at position 3 of compound 272 is displaced by the hydroxy at position 4 of compound 272 to produce epoxide compound 273. Typically the displacement is catalyzed by a suitable base, more typically, an amine base such as DBU or DBN.

Typically the process comprises treating compound 272 with a basic catalyst, optionally in the presecnce of a suitable solvent. Still more typically, the process comprises treating compound 272 with an amine base in a polar, non-protic solvent such as diethyl ether or THF. More typically yet, the process comprises treating compound 272 with DBU in THF to produce compound 273.

An exemplary embodiment of this process is given as Example 58 below.

Process E, Scheme 37

Quinic acid is used to prepare compound 274 by the following process.

The cis-4,5-diol function of quinic acid is differentiated from the carboxylic acid at carbon 1 by selective protection of these two functionalities. Typically the cis-4,5-diol function is protected as a cyclic ketal and the carboxylic acid function is protected as a lactone with the hydroxy group at position 3.

R₅₀ is as described above.

Typically, the process comprises treating quinic acid with a geminal-dialkoxyalkane or geminal dialkoxycycloalkane, as described above, and an alkanone or cycloalkanone, as described above, optionally, in the presence of an acid catalyst, as described above, to form compound 274. More typically, the process comprises treating quinic acid with a geminal-dialkoxyalkane or geminal-dialkoxycycloalkane, an alkanone or cycloalkanone, and an acid catalyst to form compound 270. Still more typically, the process comprises treating quinic acid with 2,2-dimethoxypropane, acetone, and para-toluenesulfonic acid to form compound 274.

An exemplary embodiment of this process is given as Example 101 below.

Process F, Scheme 37

Compound 274 is used to prepare compound 275 by the following process.

The lactone is opened to form compound 275. Typically, the lactone is opened to produce a protected carboxylic acid at position 1 and a free hydroxy at position 3. More typically, the lactone is opened under basic conditions to produce an R₅₁ protected carboxylic acid at position 1 and a free hydroxy group at position 3.

R₅₁ is as described above.

Typically compound 274 is treated with a suitable base in a suitable protic solvent. More typically compound 275 is treated with a metal alkoxide base, such as sodium, potassium or lithium alkoxide, in an alkanol, as described above. Still more typically, compound 274 is treated with NaOMe in MeOH to produce compound 275.

An exemplary embodiment of this process is given as Example 102 below.

Process G. Scheme 37

Compound 275 is used to prepare compound 276 by the following process.

The hydroxy group at position 3 is activated, typically, activated toward displacement reactions, more typically, activated toward epoxide ring forming displacement with an alcohol at position 4.

R₅₂ is an alcohol activating group, typically, an activating group toward displacement reactions, more typically, an activating group toward epoxide ring forming displacement with an alcohol at position 4. Such groups include those typical in the art such as sulfonic acid esters, more typically, methane, benzene or toluene sulfonic acid esters. In one embodiment, R₅₂, taken together with O (i.e. —OR₅₂), is a leaving group such as those common in the art.

Typically the process comprises treating compound 275 with an acid halide to form compound 276. More typically, the process comprises treating compound 275 with a sulfonic acid halide in a suitable solvent to form compound 276. Still more typically, the process comprises treating compound 275 with a sulfonic acid halide in a suitable solvent such as an amine, optionally, in the presence of a cosolvent, such as a haloalkane, to form compound 276. More typically yet, the process comprises treating compound 275 with p-toluene sulfonyl chloride in pyridine dichloromethane to form compound 276.

An exemplary embodiment of this process is given as Example 103 below.

Process H, Scheme 37

Compound 276 is used to prepare compound 272 by the following process.

The hydroxy group at position 1 is eliminated and the cis-4,5-diol protecting group is removed. The hydroxy group at position 1 is eliminated to form an olefinic bond between positions 1 and 6 and the cis-4,5-diol protecting group is removed to regenerate the cis-4,5-diol.

Typically the process comprises treating compound 276 with a suitable dehydrating agent, such as a mineral acid (HCl, H₂SO₄) or SO₂Cl₂. More typically, compound 276 is treated with SO₂Cl₂, followed by an alkanol, optionally in the presence of an acid catalyst. Still more typically, compound 276 is treated with SO₂Cl₂ in a suitable polar, aprotic solvent, such as an amine to form an olefin; the olefin is treated with an alkanol, as described above, and an acid catalyst, as described above, to form compound 272. More typically yet, compound 276 is treated with SO₂Cl₂ in pyridine/CH₂Cl₂ at a temperature between −100° C. and 0° C., typically −100° C. and -10° C., more typically −78° C., to form an olefin; the olefin is treated with methanol and para-toluene sulfonic acid to form compound 272.

An exemplary embodiment of this process is given as Example 104 below.

Process I, Scheme 38

Compound 273 is used to prepare compound 277 by the following process.

The hydroxy group at position 5 is protected. Typically the protecting group is an acid labile hydroxy protecting. More typically, the protecting group resists transfer to adjacent hydroxy groups.

R₅₃ is an acid labile hydroxy protecting group such as those described in the above cited work of Greene. More typically, R₅₃ is an acid cleavable ether, still more typically, R₅₃ is methoxymethyl (MOM, CH₃—O—CH₂—).

Typically the process comprises treating compound 273 with a hydroxy protecting group reagent as described in Greene. More typically the process comprises treating compound 273 with a substituted or unsubstituted haloalkane or alkene, such as methoxymethyl chloride (MOM chloride, CH₃—O—CH₂—Cl), in a suitable solvent, such as a polar, aprotic solvent. Still more typically, the process comprises treating compound 273 with MOM chloride in an amine solvent. More typically yet, the process comprises treating compound 273 with MOM chloride in diisoproply ethyl amine.

An exemplary embodiment of this process is given as Example 59 below.

Process I, Scheme 38

Compound 277 is used to prepare compound 278 by the following process.

The epoxide at positions 3 and 4 is opened to form an azide. More typically, the epoxide at positions 3 and 4 is opened to form a 3-azido-4-hydroxy compound 278.

Typically the process comprises treating compound 277 with an azide salt in a suitable solvent. More typically, the process comprises treating compound 277 with sodium azide and a mild base, such as an ammonium halide, in a polar, protic solvent, such as an alkanol or water. Still more typically, the process comprises treating compound 277 with sodium azide and ammonium chloride in water/methanol solution to produce compound 278.

An exemplary embodiment of this process is given as Example 60 below.

Process K, Scheme 38

Compound 278 is used to prepare compound 279 by the following process.

The hydroxy group at position 4 of compound 278 is displaced by the 3-azido group to form the aziridine compound 279.

Typically the process comprises treating compound 278 with a hydroxy activating group as described above, an organophosphine and a base. More typically the process comprises treating compound 278 with a sulfonic acid halide, such as those described above, to form an activated hydroxy compound, treating the activated hydroxy compound with trialkyl or tri arylphosphine, such as triphenylphosphine, to form a phosphonium salt, and treating the phosphonium salt with a base, such as an amine, to form compound 279. Still more typically, the process comprises treating compound 278 with mesyl chloride, to form an activated hydroxy compound, treating the activated hydroxy compound with triphenylphosphine, to form a phosphonium salt, and treating the phosphonium salt with triethylamine and H₂O, to form compound 279.

An exemplary embodiment of this process is given as Examples 61 and 62 below.

Process L, Scheme 38

Compound 279 is used to prepare compound 280 by the following process.

The aziridine compound 279 is opened with azide to form azido amine 280.

Typically the process comprises treating compound 279 with with an azide salt in a suitable solvent. More typically, the process comprises treating compound 279 with sodium azide and a mild base, such as an ammonium halide, in a polar, aprotic solvent, such as an ether, amine, or amide. Still more typically, the process comprises treating compound 279 with sodium azide and ammonium chloride in DMF solution to produce compound 280.

An exemplary embodiment of this process is given as Example 63 below.

Process M, Scheme 38

Compound 280 is used to prepare compound 281 by the following process.

The protected hydroxy group at position 5 is displaced by the amine at position 4 to form aziridine 281. Typically the aziridine 281 is substituted with an acid labile group, more typically an aziridine activating group.

R₅₄ is an acid labile group, typically an acid labile amine protecting group such as those described in the above cited work of Greene. More typically, R₅₄ is an aziridine activating group, still more typically, a group capable of activating an aziridine toward acid catalyzed ring opening. Typical R₅₄ groups include by way of example and not limitation, a linear or branched 1-oxo-alk-1-yl group of 1 to 12 carbons wherein the alkyl portion is a 1 to 11 carbon linear or branched chain alkyl group (such as CH₃(CH₂)_{z}C(O)—, z is an integer from 0 to 10, i.e. acetyl CH₃C(O)—, etc.), substituted methyl (e.g. triphenylmethyl, Ph₃C—, trityl, Tr), or a carbamate such as BOC or Cbz or a sulfonate (e.g. alkyl sulphonates such as methyl sulphonate). More typical R₅₄ groups include triphenylmethyl and 1-oxo-alk-1-yl groups having 1 to 8, still more typically, 1, 2, 3, 4, 5, or 6, more typically yet, 2 or 3 carbon atoms.

Typically the process comprises treating compound 280 with a deprotecting agent to remove group R₅₃, an R₅₄ producing reagent such as those described in Greene (R₅₄-halide, such as acetylchloride, or Tr-Cl, or R₅₄—O—R₅₄, such as acetic anhydride), and a hydroxy activating group such as those described in process B, Scheme 36. More typically the process comprises treating compound 280 with a polar, protic solvent, optionally in the presence of an acid catalyst as described above, to form a first intermediate; treating the first intermediate with Tr-Cl in a polar, aprotic solvent, such as an amine, to form a second intermediate; and treating the second intermediate with a sulfonic acid halide, such as mesyl chloride or para toluene sulfonyl chloride, in a polar aprotic solvent, such as an amine, to produce compound 281. Still more typically, the process comprises treating compound 280 with methanol and HCl, to form a first intermediate; treating the first intermediate with Tr-Cl and triethylamine, to form a second intermediate; and treating the second intermediate with mesyl chloride and triethylamine, to produce compound 281.

An exemplary embodiment of this process is given as Example 64 below.

Process N, Scheme 39

Compound 281 is used to prepare compound 282 by the following process.

Aziridine 281 is opened and the resulting amine is substituted with an R₅₅ group to form compound 282. Typically, aziridine 281 is opened by acid catalyzed ring opening and the resulting amine is acylated.

R₅₅ is W₃ as defined above. Typically R₅₅ is —C(O)R₅. More typically, R₅₅ is —C(O)R₁. Still more typically, R₅₅ is —C(O)CH₃.

R₅₆ is U₁ as described above. Typically R₅₆ is W₆—O—, W₆—S—, or W₆—N(H)—. More typically, R₅₆ is R₅—O—, R₅—S—, or R₅—N(H)—, still more tyically, R₅₆ is R₅—O—, still more typically yet, R₅₆ is R₁—O—.

Typically the process comprises treating compound 281 with an acid catalyst and a compound of the formula W₆—X₁—H, wherein X₁ is as defined above to form an amine intermediate; and treating the amine intermediate with a compound of the formula W₃—X₁—W₃, W₃—X₁₀, wherein X₁₀ is a leaving group, to form compound 282. The acid catalyst is typically a Lewis acid catalyst common in the art, such as BF₃.Et₂O, TiCl₃, TMSOTf, SmI₂(THF)₂, LiClO₄, Mg(ClO₄)₂, Ln(OTf)₃ (where Ln=Yb, Gd, Nd), Ti(Oi—Pr)₄, AlCl₃, AlBr₃, BeCl₂, CdCl₂, ZnCl₂, BF₃, BCl₃, BBr₃, GaCl₃, GaBr₃, TiCl₄, TiBr₄, ZrCl₄, SnCl₄, SnBr₄, SbCl₅, SbCl₃, BiCl₃, FeCl₃, UCl₄, ScCl₃, YCl₃, LaCl₃, CeCl₃, PrCl₃, NdCl₃, SmCl₃, EuCl₃, GdCl₃, TbCl₃, LuCl₃, DyCl₃, HoCl₃, ErCl₃, TmCl₃, YbCl₃, ZnI₂, Al(OPrⁱ)₃, Al(acac)₃, ZnBr₂, for SnCl₄. X₁ is typically —O—, —S—, or —N(H)—. X₁₀ is typically a halide such as Cl, Br, or I. More typically, the process comprises treating compound 281 with a compound of the formula R₅—OH, R₅—SH, or R₅—NH₂, and BF₃.Et₂O to form an intermediate; and treating the intermediate with an alkanoic acid anhydride to form compound 282. Still more typically, the process comprises treating compound 281 with a compound of the formula R₅—OH and BF₃.Et₂O to form an intermediate; and treating the intermediate with a substituted or unsubstituted acetic anhydride to form compound 282. Exemplary compounds of the formula R₅—OH include those described by Table 2, groups 2-7, 9-10, 15, and 100-660 wherein Q₁ is —OH. Further exemplary compounds of the formula R₅—OH include those shown in Table 25 below (together with their Chemical Abstracts Service Registry Numbers) and those shown in Table 26 below (together with their Chemical Abstracts Service Registry Numbers, and Aldrich Chemical Company Product Numbers). More typical exemplary compounds of the formula R₅—OH are those described by Table 2, groups 2-5, 9, and 100-141 wherein Q₁ is —OH.

In another embodiment of Process N, Scheme 39, R₅₅ is H.

Typically this process embodiment comprises treating compound 281 with an acid catalyst and a compound of the formula R₅₆—X₁—H, wherein X₁ is as defined above to form an amine intermediate to form compound 282. The acid catalyst and X₁ are as described above. More typically, the process comprises treating compound 281 with a compound of the formula R₅—OH, R₅—SH, or R₅—NH₂, and BF₃.Et₂O to form compound 282. Still more typically, the process comprises treating compound 281 with a compound of the formula R₅—OH and BF₃.Et₂O to form compound 282. Exemplary compounds of the formula R₅—OH are described above.

Exemplary embodiments of this process are given as Examples 65, 86, 92, and 95 below.

Process O, Scheme 39

Compound 282 is used to prepare compound 283 by the following process.

The azide of compound 282 is reduced to form amino compound 283.

Typically the process comprises treating compound 282 with a reducing agent to form compound 283. More typically the process comprises treating compound 282 with hydrogen gas and a catalyst (such as platinum on carbon or Lindlar's catalyst), or reducing reagents (such as a trialkyl or triaryl phosphine as described above). More typically still, the process comprises treating compound 282 with triphenylphosphine in water/THF to form compound 283.

Exemplary embodiments of this process are given as Examples 87, 93, and 96 below.

Process P, Scheme 39

Compound 283 is used to prepare compound 284 by the following process.

The carboxylic acid protecting group is removed.

Typically the process comprises treating compound 283 with a base. More typically, the process comprises treating compound 283 with a metal hydroxide in a suitable solvent such as an aprotic, polar solvent. More typically still, the process comprises treating compound 283 with aqueous potassium hydroxide in THF to produce compound 284.

Exemplary embodiments of this process are given as Examples 88, 94, and 97 below.

Process O, Scheme 40

Compound 283 is used to prepare compound 285 by the following process.

The amine is converted to a protected guanidine.

R₅₇ is a guanidine protecting group common in the art, such as BOC or Me.

Typically the process comprises treating compound 283 with a guanidylating reagent such as those common in the art. Exemplary reagents include Bis-BOC Thio-Urea aminoiminomethanesulfonic acid (Kim; et al.; “Tet. Lett.” 29(26):3183-3186 (1988) and 1-guanylpyrazoles (Bernatowicz; et al.; “Tet. Lett.” 34(21):3389-3392 (1993). More typically, the process comprises treating compound 283 with Bis-BOC Thio-Urea acid. Still more typically, the process comprises treating compound 283 with Bis-BOC Thio-Urea acid and HgCl₂ to form compound 285.

An exemplary embodiment of this process is given as Example 67 below.

Process R, Scheme 40

Compound 285 is used to prepare compound 286 by the following process.

The carboxylic acid and guanidine protecting groups are removed.

Typically the process comprises treating compound 285 with a base; followed by treating with an acid, as described above. More typically the process comprises treating compound 285 with a metal hydroxide base, described above, to form an intermediate; and treating the intermediate with acid to form compound 286. Still more typically the process comprises treating compound 285 with aqueous potassium hydroxide and THF, to form an intermediate; and treating the intermediate with TFA to form compound 286.

Process S, Scheme 40.1

Compound 287 is used to prepare compound 288 by the following process.

E₁, J₁ and J₂ of compounds 287 and 288 are as described above. Typically, E₁ is —CO₂R₅₁ as described above. Typically, J₁ is H, F, or methyl, more typically, H. Typically, J₂ is H or a linear or branched alkyl of 1 to 6 carbon atoms, more typically, H, methyl, ethyl, n-propyl, or i-propyl, still more typically, H.

R₆₀ and R₆₁ are groups capable of reacting to form the R₆₃ (defined below) substituted aziridine ring of compound 288. Typically, one of R₆₀ or R₆₁ is a primary or secondary amine, or a group capable of being converted to a primary or secondary amine. Such groups for R₆₀ and R₆₁ include by way of example and not limitation, —NH₂, —N(H)(R_{6b}), —N(R_{6b})₂, —N(H)(R₁), —N(R₁)(R_{6b}), and —N₃. The other of R₆₀ and R₆₁ is typically a group capable of being displaced by a primary or secondary amine to form an aziridine. Such groups include by way of example and not limitation, —OH, —OR₆ₐ, Br, Cl, and I. Typically, R₆₀ and R₆₁ are in a trans configuration. More typically, R₆₀ is a primary or secondary amine, or a group capable of being converted to a primary or secondary amine and R₆₁ is a group capable of being displaced by a primary or secondary amine to form an aziridine. Still more typically, R₆₀ is β-azido or β-NH₂, and R₆₁ is α-OH, α-OMesyl, or α-OTosyl.

R₆₂ is described below in Process U, Scheme 40.1.

The process comprises treating compound 287 to form compound 288. This is typically accomplished by treating compound 287 to displace R₆₁ by R₆₀. More typically, compound 287 is treated to activate R₆₁ toward displacement by R₆₀. Still more typically, compound 287 is treated to activate R₆₁ toward displacement by R₆₀, and R₆₀ is activated toward displacement of R₆₁. If both R₆₀ and R₆₁ are activated, the activations can be performed simultaneously or sequentially. If the activations are performed sequentially, they can be performed in any order, typically the activation of R₆₁ precedes the activation of R₆₀.

Activation of R₆₁ toward displacement by R₆₀ is typically accomplished by treating compound 287 with a hydroxy activating reagent such as mesyl or tosyl chloride. Activation of R₆₀ toward displacement of R₆₁ is typically accomplished by treating compound 287 to form a primary or secondary amine and treating the amine with a base. By way of example and not limitation, compound 287 is treated with a reducing agent capable of reducing an azide to an amine and a base.

In one embodiment of this process, compound 287 is treated with an R₆₁ activating reagent, and an R₆₀ activating reagent to produce compound 288. In another embodiment, compound 287 is treated in a suitable solvent with an R₆₁ activating reagent, and an R₆₀ activating reagent to produce compound 288. In another embodiment, compound 287 is treated with an R₆₁ activating reagent, an R₆₀ activating reagent, and a base to produce compound 288. In another embodiment, compound 287 is treated in a suitable solvent with an R₆₁ activating reagent, an R₆₀ activating reagent, and a base to produce compound 288. In another embodiment, compound 287 wherein R₆₀ is an azide is treated with an R₆₁ activating reagent, and an azide reducing reagent to produce compound 288. In another embodiment, compound 287 wherein R₆₀ is an azide is treated in a suitable solvent with an R₆₁ activating reagent, and an azide reducing reagent to produce compound 288. In another embodiment, compound 287 wherein R₆₀ is an azide is treated with an R₆₁ activating reagent, an azide reducing reagent, and a base to produce compound 288. In another embodiment, compound 287 wherein R₆₀ is an azide is treated in a suitable solvent with an R₆₁ activating reagent, an azide reducing reagent, and a base to produce compound 288. In another embodiment, compound 287 wherein R₆₀ is an azide and R₆₁ is a hydroxy, is treated with a hydroxy activating reagent, and an azide reducing reagent to produce compound 288. In another embodiment, compound 287 wherein R₆₀ is an azide and R₆₁ is a hydroxy, is treated in a suitable solvent with an hydroxy activating reagent, and an azide reducing reagent to produce compound 288. In another embodiment, compound 287 wherein R₆₀ is an azide and R₆₁ is a hydroxy, is treated with a hydroxy activating reagent, an azide reducing reagent, and a base to produce compound 288. In another embodiment, compound 287 wherein R₆₀ is an azide and R₆₁ is a hydroxy, is treated in a suitable solvent with a hydroxy activating reagent, an azide reducing reagent, and a base to produce compound 288.

An exemplary embodiments of this process are given as Process K, Scheme 38, above.

Process T, Scheme 40.1

Compound 288 is used to prepare compound 289 by the following process.

R₆₄ is typically H, R_{6b} or a group capable of being converted to H or R_{6b}. More typically, R₆₄ is H. R₆₅ is typically G₁ or a group capable of being converted to G₁. More typically, R₆₅ is —N₃, —CN, or —(CR₁R₁)ₘ₁W₂. More typically R₆₅ is —N₃, —NH₂, —N(H)(R_{6b}), —N(R_{6b})₂, —CH₂N₃, or —CH₂CN.

Typically, compound 288 is treated to form amine 289. More typically, compound 288 is treated with a nucleophile, typically a nitrogen nucleophile such as R₆₅, a cationic salt of R₆₅, or a protonated analog of R₆₅, such as by way of example and not limitation, NH₃, an azide salt (such as NaN₃, KN₃, or the like), HCN, a cyanide salt (such as NaCN, KCN, or the like), or a salt of a cyanoalkyl (e.g. (CH₂CN)⁻) (such as NaCH₂CN, KCH₂CN, or the like). Still more typically, compound 288 is treated with an azide salt. Optionally a base, typically a mild base such as an ammonium halide and a solvent, typically a polar, aprotic solvent, such as an ether, amine, or amide are used.

In one embodiment, compound 288 is treated with a nucleophile. In another embodiment, compound 288 is treated with a nucleophile in a suitable solvent to produce compound 289. In another embodiment, compound 288 is treated with a nucleophile and a base to produce compound 289. In another embodiment, compound 288 is treated with a nucleophile and a base in a suitable solvent to produce compound 289. In another embodiment, compound 288 is treated with a nitrogen nucleophile to produce compound 289. In another embodiment, compound 288 is treated with a nitrogen nucleophile in a suitable solvent to produce compound 289. In another embodiment, compound 288 is treated with a nitrogen nucleophile and a base to produce compound 289. In another embodiment, compound 288 is treated with a nitrogen nucleophile and a base in a suitable solvent to produce compound 289. In another embodiment, compound 288 is treated with an azide salt to produce compound 289. In another embodiment, compound 288 is treated with an azide salt in a suitable solvent to produce compound 289. In another embodiment, compound 288 is treated with an azide salt and a base to produce compound 289. In another embodiment, compound 288 is treated with an azide salt and a base in a suitable solvent to produce compound 289.

An exemplary embodiment of this process is given as Process L, Scheme 38, above.

Process U, Scheme 40.1

Compound 289 is used to prepare compound 290 by the following process.

R₆₂ is a group capable of reacting with an amine to form the R₆₆ (defined below) substituted aziridine ring of compound 290. Typically, R₆₂ is a group capable of being displaced by a primary or secondary amine to form an aziridine. Such groups include by way of example and not limitation, —OR₅₃, —OH, —OR₆ₐ, Br, Cl, and I. Typically, R₆₂ is in a trans configuration relative to the nitrogen in position 4. More typically, R₆₂ is —OR₅₃.

R₆₄ is H or R_{6b}, typically an acid labile protecting group such as R₅₄.

R₆₆ is H, R_{6b} or R₅₄.

The process comprises treating compound 289 to form compound 290. This is typically accomplished by treating compound 289 to displace R₆₂ by the amine at position 4. More typically, compound 289 is treated to activate the amine at position 4 toward displacement of R₆₂. Still more typically, compound 289 is treated to activate the amine at position 4 toward displacement of R₆₂, and R₆₂ is activated toward displacement by the amine at position 4. If both R₆₂ and the amine at position 4 are activated, the activations can be performed simultaneously or sequentially. If the activations are performed sequentially, they can be performed in any order, typically the activation of R₆₂ precedes the activation of the amine at position 4.

Activation of R₆₂ toward displacement by the amine at position 4 is typically accomplished by treating compound 289 with a hydroxy activating agent such as those described in process B, Scheme 36. Optionally, R₆₂ is deprotected prior to activation. Activation of the amine at position 4 toward R₆₂ displacement is typically accomplished by treating compound 289 to form a primary or secondary amine and treating the amine with an acid catalyst such as those described in Process N, Scheme 39, above.

Typically when R₆₂ is —OR₅₃ and R₆₆ is R₅₆, the process comprises treating compound 289 with a deprotecting agent to remove group R₅₃, an R₅₄ producing reagent such as those described in Greene (R₅₄-halide, such as acetylchloride, or Tr-Cl, or R₅₄—O—R₅₄, such as acetic anhydride), and a hydroxy activating group such as those described in Process B, Scheme 36. More typically the process comprises treating compound 289 with a polar, protic solvent, optionally in the presence of an acid catalyst as described above, to form a first intermediate; treating the first intermediate with Tr-Cl in a polar, aprotic solvent, such as an amine, to form a second intermediate; and treating the second intermediate with a sulfonic acid halide, such as mesyl chloride or para toluene sulfonyl chloride, in a polar aprotic solvent, such as an amine, to produce compound 290. Still more typically, the process comprises treating compound 289 with methanol and HCl, to form a first intermediate; treating the first intermediate with Tr-Cl and triethylamine, to form a second intermediate; and treating the second intermediate with mesyl chloride and triethylamine, to produce compound 290.

In one embodiment compound 289 is treated with an acid catalyst to produce compound 290. In another embodiment compound 289 is treated with an acid catalyst in a suitable solvent to produce compound 290. In another embodiment compound 289 is treated with a hydroxy activating reagent and an acid catalyst to produce compound 290. In another embodiment compound 289 is treated with a hydroxy activating reagent and an acid catalyst in a suitable solvent to produce compound 290. In another embodiment compound 289 is treated with a hydroxy deprotecting reagent, a hydroxy activating reagent and an acid catalyst to produce compound 290. In another embodiment compound 289 is treated with a hydroxy activating reagent and an acid catalyst in a suitable solvent to produce compound 290.

An exemplary embodiment of this process is given as Process M, Scheme 38, above.

Process V, Scheme 40.1

Compound 290 is used to prepare compound 291 by the following process.

Aziridine 290 is treated to form compound 291. Typically, aziridine 290 is opened by acid catalyzed ring opening and the resulting amine is acylated.

R₆₈ is independently H, R_{6b}, R₁ or R₅₅ as defined above. Typically R₅₅ is —C(O)R₅. Typically one R₆₈ is H or R_{6b} and the other is W₃.

R₆₇ is U₁ as described above. Typically R₆₇ is W₆—O—, W₆—S—, or W₆—N(H)—. More typically, R₆₇ is R₅—O—, R₅—S—, or R₅—N(H)—.

Typically the process comprises treating compound 290 with an acid catalyst and a compound of the formula W₆—X₁—H, wherein X₁ is as defined above to form an amine intermediate; and treating the amine intermediate with a compound of the formula W₃—X₁—W₃, or W₃—X₁₀, wherein X₁₀ is a leaving group, to form compound 291. The treatment with a compound of the formula W₆—X1—H and an acid catalyst may be prior to or simultaneous with the treatment with a compound of the formula W₃—X₁—W₃, or W₃—X₁₀. The acid catalyst is typically one of those described in Process N, Scheme 39, above. More typically, the process comprises treating compound 290 with a compound of the formula R₅—OH, R₅—SH, or R₅—NH₂ and an acid catalyst; and treating the intermediate with an alkanoic acid anhydride to form compound 291.

One embodiment comprises treating compound 290 with a compound of the formula W₆—X₁—H and an acid catalyst to produce compound 291. Another embodiment comprises treating compound 290 with a compound of the formula W₆—X₁—H and an acid catalyst in a suitable solvent to produce compound 291. Another embodiment comprises treating compound 290 with a compound of the formula W₆—X₁—H, an acid catalyst and a compound of the formula W₃—X₁—W₃ or W₃—X₁₀ to produce compound 291. Another embodiment comprises treating compound 290 with a compound of the formula W₆—X₁—H, an acid catalyst and a compound of the formula W₃—X₁—W₃ or W₃—X₁₀ in a suitable solvent to produce compound 291.

Exemplary embodiments of this process are given as Process N, Scheme 39, above.

Process W, Scheme 40.1

Compound 291 is used to prepare compound 292 by the following process.

Compound 291 is treated to form compound 292. Typically R₆₅ is converted to form G₁. U₁ is an embodiment of R₆₇ and T₁ is an embodiment of —N(R₆₈)₂ prepared in Process V, Scheme 40.1, above.

In one embodiment, R₆₅ is deprotected, alkylated, guanidinylated, oxidized or reduced to form G₁. Any number of such treatments can be performed in any order or simultaneously. By way of example and not limitation, when R₆₅ is azido, embodiments of this process include Processes O, OQ, OQR, and OP. Typical alkylating agents are those common in the art including, by way of example and not limitation, an alkyl halide such as methyl iodide, methyl bromide, ethyl iodide, ethyl bromide, n-propyl iodide, n-propyl bromide, i-propyl iodide, i-propyl bromide; and an olefin oxide such as ethylene oxide or propylene oxide. A base catalyst as described herein maybe optionally employed in the alkylation step.

One embodiment comprises treating compound 291 wherein R₆₅ is azido with a reducing agent to produce compound 292. Another embodiment comprises treating compound 291 wherein R₆₅ is azido with a reducing agent to produce compound 292 in a suitable solvent. Another embodiment comprises treating compound 291 wherein R₆₅ is amino with an alkylating agent to produce compound 292. Another embodiment comprises treating compound 291 wherein R₆₅ is amino with an alkylating agent to produce compound 292 in a suitable solvent. Another embodiment comprises treating compound 291 wherein R₆₅ is azido with a reducing agent and an alkylating agent to produce compound 292. Another embodiment comprises treating compound 291 wherein R₆₅ is azido with a reducing agent and an alkylating agent to produce compound 292 in a suitable solvent. Another embodiment comprises treating compound 291 wherein R₆₅ is amino with an alkylating agent and a base catalyst to produce compound 292. Another embodiment comprises treating compound 291 wherein R₆₅ is amino with an alkylating agent and a base catalyst to produce compound 292 in a suitable solvent. Another embodiment comprises treating compound 291 wherein R₆₅ is azido with a reducing agent, an alkylating agent and a base catalyst to produce compound 292. Another embodiment comprises treating compound 291 wherein R₆₅ is azido with a reducing agent, an alkylating agent and a base catalyst to produce compound 292 in a suitable solvent.

Exemplary embodiments of this process are given as Process O, Scheme 39, above.

Exemplary embodiments of this process are given as Examples 68 and 69 below.

TABLE 25
Exemplary Compounds of Formula R₅—OH (CAS No.)

C4 Fluoro Alcohols
(R*,R*)-(±)-3-fluoro-2-Butanol (139755-61-6)
1-fluoro-2-Butanol (124536-12-5)
(R)-3-fluoro-1-Butanol (120406-57-7)
3-fluoro-1-Butanol (19808-95-8)
4-fluoro-2-Butanol (18804-31-4)
(R*,S*)-3-fluoro-2-Butanol (6228-94-0)
(R*,R*)-3-fluoro-2-Butanol (6133-82-0)
2-fluoro-1-Butanol (4459-24-9)
2-fluoro-2-methyl-1-Propanol (3109-99-7)
3-fluoro-2-Butanol (1813-13-4)
4-fluoro-1-Butanol (372-93-0)
1-fluoro-2-methyl-2-Propanol (353-80-0)
C5 Fluoro Alcohols
2-fluoro-1-Pentanol (123650-81-7)
(R)-2-fluoro-3-methyl-1-Butanol (113943-11-6)
(S)-2-fluoro-3-methyl-1-Butanol (113942-98-6)
4-fluoro-3-methyl-1-Butanol (104715-25-5)
1-fluoro-3-Pentanol (30390-84-2)
4-fluoro-2-Pentanol (19808-94-7)
5-fluoro-2-Pentanol (18804-35-8)
3-fluoro-2-methyl-2-Butanol (7284-96-0)
2-fluoro-2-methyl-1-Butanol (4456-02-4)
3-fluoro-3-methyl-2-Butanol (1998-77-2)
5-fluoro-1-Pentanol (592-80-3)
C6 Fluoro Alcohols
(R-(R*,S*))-2-fluoro-3-methyl-1-Pentanol (168749-88-0)
1-fluoro-2,3-dimethyl-2-Butanol (161082-90-2)
2-fluoro-2,3-dimethyl-1-Butanol (161082-89-9)
(R)-2-fluoro-4-methyl-1-Pentanol (157988-30-2)
(S-(R*,R*))-2-fluoro-3-methyl-1-Pentanol (151717-18-9)
(R*,S*)-2-fluoro-3-methyl-1-Pentanol (151657-14-6)
(S)-2-fluoro-3,3-dimethyl-1-Butanol (141022-94-8)
(M)-2-fluoro-2-methyl-1-Pentanol (137505-57-8)
(S)-2-fluoro-1-Hexanol (127608-47-3)
3-fluoro-3-methyl-1-Pentanol (112754-22-0)
3-fluoro-2-methyl-2-Pentanol (69429-54-5)
2-fluoro-2-methyl-3-Pentanol (69429-53-4)
1-fluoro-3-Hexanol (30390-85-3)
5-fluoro-2-methyl-2-Pentanol (21871-78-3)
5-fluoro-3-Hexanol (19808-92-5)
4-fluoro-3-methyl-2-Pentanol (19808-90-3)
4-fluoro-4-methyl-2-Pentanol (19031-69-7)
1-fluoro-3,3-dimethyl-2-Butanol (4604-66-4)
2-fluoro-2-methyl-1-Pentanol (4456-03-5)
2-fluoro-4-methyl-1-Pentanol (4455-95-2)
2-fluoro-1-Hexanol (1786-48-7)
3-fluoro-2,3-dimethyl-2-Butanol (661-63-2)
6-fluoro-1-Hexanol (373-32-0)
C7 Fluoro Alcohols
5-fluoro-5-methyl-1-Hexanol (168268-63-1)
(R)-1-fluoro-2-methyl-2-Hexanol (153683-63-7)
(S)-3-fluoro-1-Heptanol (141716-56-5)
(S)-2-fluoro-2-methyl-1-Hexanol (132354-09-7)
(R)-3-fluoro-1-Heptanol (120406-54-4)
(S)-2-fluoro-1-Heptanol (110500-31-7)
1-fluoro-3-Heptanol (30390-86-4)
7-fluoro-2-Heptanol (18804-38-1)
2-ethyl-2-(fluoromethyl)-1-Butanol (14800-35-2)
2-(fluoromethyl)-2-methyl-1-Pentanol (13674-80-1)
2-fluoro-5-methyl-1-Hexanol (4455-97-4)
2-fluoro-1-Heptanol (1786-49-8)
7-fluoro-1-Heptanol (408-16-2)
C8 Fluoro Alcohols
(M)-2-fluoro-2-methyl-1-Heptanol (137505-55-6)
6-fluoro-6-methyl-1-Heptanol (135124-57-1)
1-fluoro-2-Octanol (127296-11-1)
(R)-2-fluoro-1-Octanol (118205-91-7)
(±)-2-fluoro-2-methyl-1-Heptanol (117169-40-1)
(S)-2-fluoro-1-Octanol (110500-32-8)
(S)-1-fluoro-2-Octanol (110270-44-5)
(R)-1-fluoro-2-Octanol (110270-42-3)
(±)-1-fluoro-2-Octanol (110229-70-4)
2-fluoro-4-methyl-3-Heptanol (87777-41-1)
2-fluoro-6-methyl-1-Heptanol (4455-99-6)
2-fluoro-1-Octanol (4455-93-0)
8-fluoro-1-Octanol (408-27-5)
C9 Fluoro Alcohols
6-fluoro-2,6-dimethyl-2-Heptanol (160981-64-6)
(S)-3-fluoro-1-Nonanol (160706-24-1)
(R-(R*,R*))-3-fluoro-2-Nonanol (137909-46-7)
(R-(R*,S*))-3-fluoro-2-Nonanol (137909-45-6)
3-fluoro-2-Nonanol (137639-20-4)
(S-(R*,R*))-3-fluoro-2-Nonanol (137639-19-1)
(S-(R*,S*))-3-fluoro-2-Nonanol (137639-18-0)
(±)-3-fluoro-1-Nonanol (134056-76-1)
2-fluoro-1-Nonanol (123650-79-3)
2-fluoro-2-methyl-1-Octanol (120400-89-7)
(R)-2-fluoro-1-Nonanol (118243-18-8)
(S)-1-fluoro-2-Nonanol (111423-41-7)
(S)-2-fluoro-1-Nonanol (110500-33-9)
1-fluoro-3-Nonanol (30390-87-5)
2-fluoro-2,6-dimethyl-3-Heptanol (684-74-2)
9-fluoro-1-Nonanol (463-24-1)
C10 Fluoro Alcohols
4-fluoro-1-Decanol (167686-45-5)
(P)-10-fluoro-3-Decanol (145438-91-1)
(R-(R*,R*))-3-fluoro-5-methyl-1-Nonanol (144088-79-9)
(P)-10-fluoro-2-Decanol (139750-57-5)
1-fluoro-2-Decanol (130876-22-1)
(S)-2-fluoro-1-Decanol (127608-48-4)
(R)-1-fluoro-2-Decanol (119105-16-7)
(S)-1-fluoro-2-Decanol (119105-15-6)
2-fluoro-1-Decanol (110500-35-1)
1-fluoro-5-Decanol (106533-31-7)
4-fluoro-2,2,5,5-tetramethyl-3-Hexanol (24212-87-1)
10-fluoro-1-Decanol (334-64-5)
C11 Fluoro Alcohols
10-fluoro-2-methyl-1-Decanol (139750-53-1)
2-fluoro-1-Undecanol (110500-34-0)
8-fluoro-5,8-dimethyl-5-Nonanol (110318-90-6)
11-fluoro-2-Undecanol (101803-63-8)
11-fluoro-1-Undecanol (463-36-5)
C12 Fluoro Alcohols
11-fluoro-2-methyl-1-Undecanol (139750-52-0)
1-fluoro-2-Dodecanol (132547-33-2)
(R*,S*)-7-fluoro-6-Dodecanol (130888-52-7)
(R*,R*)-7-fluoro-6-Dodecanol (130876-18-5)
(S)-2-fluoro-1-Dodecanol (127608-49-5)
12-fluoro-2-pentyl-Heptanol (120400-91-1)
(R*,S*)-(±)-7-fluoro-6-Dodecanol (119174-39-9)
(R*,R*)-(±)-7-fluoro-6-Dodecanol (119174-38-8)
2-fluoro-1-Dodecanol (110500-36-2)
11-fluoro-2-methyl-2-Undecanol (101803-67-2)
1-fluoro-1-Dodecanol (100278-87-3)
12-fluoro-1-Dodecanol (353-31-1)
C4 Nitro Alcohols
(R)-4-nitro-2-Butanol (129520-34-9)
(S)-4-nitro-2-Butanol (120293-74-5)
4-nitro-1-Butanol radical ion(1-) (83051-13-2)
(R*,S*)-3-nitro-2-Butanol (82978-02-7)
(R*,R*)-3-nitro-2-Butanol (82978-01-6)
4-nitro-1-Butanol (75694-90-5)
(±)-4-nitro-2-Butanol (72959-86-5)
4-nitro-2-Butanol (55265-82-2),
1-aci-nitro-2-Butanol (22916-75-2)
3-aci-nitro2-Butanol (22916-74-1)
2-methyl-3-nitro-1-Propanol (21527-52-6)
3-nitro-2-Butanol (6270-16-2)
2-methyl-1-nitro-2-Propanol (5447-98-3)
2-aci-nitro-1-Butanol (4167-97-9)
1-nitro-2-Butanol (3156-74-9)
2-nitro-1-Butanol (609-31-4)
2-methyl-2-nitro-1-Propanol (76-39-1)
C5 Nitro Alcohols
(R)-3-methyl-3-nitro-2-Butanol (154278-27-0)
3-methyl-1-nitro-1-Butanol (153977-20-9)
(±)-1-nitro-3-Pentanol (144179-64-6)
(S)-1-nitro-3-Pentanol (144139-35-5)
(R)-1-nitro-3-Pentanol (144139-34-4)
(R)-3-methyl-1-nitro-2-Butanol (141434-98-2)
(±)-3-methyl-1-nitro-2-Butanol (141377-55-1)
(R*,R*)-3-nitro-2-Pentanol (138751-72-1)
(R*,S*)-3-nitro-2-Pentanol (138751-71-0)
(R*,R*)-2-nitro-3-Pentanol (138668-26-5)
(R*,S*)-2-nitro-3-Pentanol (138668-19-6)
3-nitro-1-Pentanol (135462-98-5)
(R)-5-nitro-2-Pentanol (129520-35-0)
(S)-5-nitro-2-Pentanol (120293-75-6)
4-nitro-1-Pentanol (116435-64-4)
(±)-3-methyl-3-nitro-2-Butanol (114613-30-8)
(S)-3-methyl-3-nitro-2-Butanol (109849-50-5)
3-methyl-4-nitro-2-Butanol (96597-30-7)
(±)-5-nitro-2-Pentanol (78174-81-9)
2-methyl-2-nitro-1-Butanol (77392-55-3)
3-methyl-2-nitro-1-Butanol (77392-54-2)
3-methyl-4-nitro-1-Butanol (75694-89-2)
2-methyl-4-nitro-2-Butanol (72183-50-7)
3-methyl-3-nitro-1-Butanol (65102-50-3)
5-nitro-2-Pentanol (54045-33-9)
2-methyl-3-aci-nitro-2-Butanol (22916-79-6)
2-methyl-1-aci-nitro-2-Butanol (22916-78-5)
2-methyl-3-nitro-2-Butanol (22916-77-4)
2-methyl-1-nitro-2-Butanol (22916-76-3)
5-nitro-1-Pentanol (21823-27-8)
2-methyl-3-nitro-1-Butanol (21527-53-7)
2-nitro-3-Pentanol (20575-40-0)
3-methyl-3-nitro-2-Butanol (20575-38-6)
3-nitro-2-Pentanol (5447-99-4)
2-nitro-1-Pentanol (2899-90-3)
3-methyl-1-nitro-2-Butanol (2224-38-6)
1-nitro-2-Pentanol (2224-37-5)
C6 Nitro Alcohols
(−)-4-methyl-1-nitro-2-Pentanol (158072-33-4)
3-(nitromethyl)-3-Pentanol (156544-56-8)
(R*,R*)-3-methyl-2-nitro-3-Pentanol (148319-17-9)
(R*,S*)-3-methyl-2-nitro-3-Pentanol (148319-16-8)
6-nitro-2-Hexanol (146353-95-9)
(±)-6-nitro-3-Hexanol (144179-63-5)
(S)-6-nitro-3-Hexanol (144139-33-3)
(R)-6-nitro-3-Hexanol (144139-32-2)
3-nitro-2-Hexanol (127143-52-6)
5-nitro-2-Hexanol (110364-37-9)
4-methyl-1-nitro-2-Pentanol (102014-44-8)
(R*,S*)-2-methyl-4-nitro-3-Pentanol (82945-29-7)
(R*,R*)-2-methyl-4-nitro-3-Pentanol (82945-20-8)
2-methyl-5-nitro-2-Pentanol (79928-61-3)
2,3-dimethyl-1-nitro-2-Butanol (68454-59-1)
2-methyl-3-nitro-2-Pentanol (59906-62-6)
3,3-dimethyl-1-nitro-2-Butanol (58054-88-9)
2,3-dimethyl-3-nitro-2-Butanol (51483-61-5)
2-methyl-1-nitro-2-Pentanol (49746-26-1)
3,3-dimethyl-2-nitro-1-Butanol (37477-66-0)
6-nitro-1-Hexanol (31968-54-4)
2-methyl-3-nitro-1-Pentanol (21527-55-9)
2,3-dimethyl-3-nitro-1-Butanol (21527-54-8)
2-methyl-4-nitro-3-Pentanol (20570-70-1)
2-methyl-2-nitro-3-Pentanol (20570-67-6)
2-nitro-3-Hexanol (5448-00-0)
4-nitro-3-Hexanol (5342-71-2)
4-methyl-4-nitro-1-Pentanol (5215-92-9)
1-nitro-2-Hexanol (2224-40-0)
C7 Nitro Alcohols
1-nitro-4-Heptanol (167696-66-4)
(R)-1-nitro-2-Heptanol (146608-19-7)
7-nitro-1-Heptanol (133088-94-5)
(R*,S*)-3-nitro-2-Heptanol (127143-73-1)
(R*,R*)-3-nitro-2-Heptanol (127143-72-0)
(R*,S*)-2-nitro-3-Heptanol (127143-71-9)
(R*,R*)-2-nitro-3-Heptanol (127143-70-8)
(R*,S*)-2-methyl-5-nitro-3-Hexanol (103077-95-8)
(R*,R*)2-methyl-5-nitro-3-Hexanol (103077-87-8)
3-ethyl-4-nitro-1-Pentanol (92454-38-1)
3-ethyl-2-nitro-3-Pentanol (77922-54-4)
2-nitro-3-Heptanol (61097-77-6)
2-methyl-1-nitro-3-Hexanol (35469-17-1)
2-methyl-4-nitro-3-Hexanol (20570-71-2)
2-methyl-2-nitro-3-Hexanol (20570-69-8)
5-methyl-5-nitro-2-Hexanol (7251-87-8)
1-nitro-2-Heptanol (6302-74-5)
3-nitro-4-Heptanol (5462-04-4)
4-nitro-3-Heptanol (5342-70-1)
C8 Nitro Alcohols
(±)-1-nitro-3-Octanol (141956-93-6)
1-nitro-4-Octanol (167642-45-7)
(S)-1-nitro-4-Octanol (167642-18-4)
6-methyl-6-nitro-2-Heptanol (142991-77-3)
(R*,S*)-2-nitro-3-Octanol (135764-74-8)
(R*,R*)-2-nitro-3-Octanol (135764-73-7)
5-nitro-4-Octanol (132272-46-9)
(R*,R*)-3-nitro-4-Octanol (130711-79-4)
(R*,S*)-3-nitro-4-Octanol (130711-78-3)
4-ethyl-2-nitro-3-Hexanol (126939-74-0)
2-nitro-3-Octanol (126939-73-9)
1-nitro-3-Octanol (126495-48-5)
(R*,R*)-(±)-3-nitro-4-Octanol (118869-22-0)
(R*-S*)-(±)-3-nitro-4-Octanol (118869-21-9)
3-nitro-2-Octanol (127143-53-7)
(R*,S*)-2-methyl-5-nitro-3-Heptanol-(103078-03-1)
(R*,R*)-2-methyl-5-nitro-3-Heptanol-(103077-90-3)
8-nitro-1-Octanol (101972-90-1)
(±)-2-nitro-1-Octanol (96039-95-1)
3,4-dimethyl-1-nitro-2-Hexanol (64592-02-5)
3-(nitromethyl)-4-Heptanol (35469-20-6)
2,5-dimethyl-1-nitro-3-Hexanol (35469-19-3)
2-methyl-1-nitro-3-Heptanol (35469-18-2)
2,4,4-trimethyl-1-nitro-2-Pentanol (35223-67-7)
2,5-dimethyl-4-nitro-3-Hexanol (22482-65-1)
2-nitro-1-Octanol (2882-67-9)
1-nitro-2-Octanol (2224-39-7)
C9 Nitro Alcohols
4-nitro-3-Nonanol (160487-89-8)
(R*,R*)-3-ethyl-2-nitro-3-Heptanol (148319-18-0)
2,6-dimethyl-6-nitro-2-Heptanol (117030-50-9)
(R*,S*)-2-nitro-4-Nonanol (103077-93-6)
(R*,R*)-2-nitro-4-Nonanol (103077-85-6)
2-nitro-3-Nonanol (99706-65-7)
9-nitro-1-Nonanol (81541-84-6)
2-methyl-1-nitro-3-Octanol (53711-06-1)
4-nitro-5-Nonanol (34566-13-7)
2-methyl-3-(nitromethyl)-3-Heptenol (5582-88-7)
1-nitro-2-Nonanol (4013-87-0)
C10 Nitro Alcohols
2-nitro-4-Decanol (141956-94-7)
(R*,S*)-3-nitro-4-Decanol (135764-76-0)
(R*,R*)-3-nitro-4-Decanol (135764-75-9)
5,5-dimethyl-4-(2-nitroethyl)-1-Hexanol (133088-96-7)
(R*,R*)-(±)-3-nitro-4-Decanol (118869-20-8)
(R*,S*)-(±)-3-nitro-4-Decanol (118869-19-5)
5-nitro-2-Decanol (112882-29-8)
3-nitro-4-Decanol (93297-82-6)
4,6,6-trimethyl-1-nitro-2-Heptanol (85996-72-1)
2-methyl-2-nitro-3-Nonanol (80379-17-5)
1-nitro-2-Decanol (65299-35-6)
2,2,4,4-tetramethyl-3-(nitromethyl)-3-Pentanol (58293-26-8)
C11 Nitro Alcohols
11-nitro-5-Undecanol (167696-69-7)
(R*,R*)-2-nitro-3-Undecanol (144434-56-0)
(R*,S*)-2-nitro-3-Undecanol (144434-55-9)
2-nitro-3-Undecanol (143464-92-0)
2,2-dimethyl-4-nitro-3-Nonanol (126939-76-2)
4,8-dimethyl-2-nitro-1-Nonanol (118304-30-6)
11-nitro-1-Undecanol (81541-83-5)
C12 Nitro Alcohols
2-methyl-2-nitro-3-Undecanol (126939-75-1)
2-nitro-1-Dodecanol (62322-32-1)
1-nitro-2-Dodecanol (62322-31-0)
2-nitro-3-Dodecanol (82981-40-6)
12-nitro-1-Dodecanol (81541-78-8)

TABLE 26
Exemplary Compounds of Formula R₅—OH (CAS No./Aldrich No.)

| | | |
|---|---|---|
| 3-BROMO-1-PROPANOL | 627189 | 167169 |
| 1,3-DICHLORO-2-PROPANOL | 96231 | 184489 |
| 3-CHLORO-2,2-DIMETHYL-1-PROPANOL | 13401564 | 189316 |
| 2,2-BIS(CHLOROMETHYL)-1-PROPANOL | 5355544 | 207691 |
| 1,3-DIFLUORO-2-PROPANOL | 453134 | 176923 |
| 2-(METHYLTHIO)ETHANOL | 5271385 | 226424 |
| 2-(DIBUTYLAMINO)ETHANOL | 102818 | 168491 |
| 2-(DIISOPROPYLAMINO)ETHANOL | 96800 | 168726 |
| 3-METHYL-3-BUTEN-1-OL | 763326 | 129402 |
| 2-METHYL-3-BUTEN-2-OL | 115184 | 136816 |
| 3-METHYL-2-BUTEN-1-OL | 556821 | 162353 |
| 4-HEXEN-1-OL | 928927 | 237604 |
| 5-HEXEN-1-OL | 821410 | 230324 |
| CIS-2-HEXEN-1-OL | 928949 | 224707 |
| TRANS-3-HEXEN-1-OL | 928972 | 224715 |
| TRANS-2-HEXEN-1-OL | 928950 | 132667 |
| (+/−)-6-METHYL-5-HEPTEN-2-OL | 4630062 | 195871 |
| DIHYDROMYRCENOL | 18479588 | 196428 |
| TRANS,TRANS-2,4-HEXADIEN-1-OL | 17102646 | 183059 |
| 2,4-DIMETHYL-2,6-HEPTADIEN-1-OL | 80192569 | 238767 |
| GERANIOL | 106241 | 163333 |
| 3-BUTYN-1-OL | 927742 | 130850 |
| 3-PENTYN-1-OL | 10229104 | 208698 |
| ISETHIONIC ACID, SODIUM SALT | 1562001 | 220078 |
| (4-(2-HYDROXYETHYL)-1-PIPERAZINE- | 16052065 | 163740 |
| PROPANESULFONIC ACID) | | |
| HEPES, SODIUM SALT | 75277393 | 233889 |
| 1-METHYLCYCLOPROPANEMETHANOL | 2746147 | 236594 |
| 2-METHYLCYCLOPROPANEMETHANOL | 6077721 | 233811 |
| (+/−)-CHRYSANTHEMYL ALCOHOL | 18383590 | 194654 |
| CYCLOBUTANEMETHANOL | 4415821 | 187917 |
| 3-CYCLOPENTYL-1-PROPANOL | 767055 | 187275 |
| 1-ETHYNYLCYCLOPENTANOL | 17356193 | 130869 |
| 3-METHYLCYCLOHEXANOL | 591231 | 139734 |
| 3,3,5,5-TETRAMETHYLCYCLOHEXANOL | 2650400 | 190624 |
| 4-CYCLOHEXYL-1-BUTANOL | 4441570 | 197408 |
| DIHYDROCARVEOL | 619012 | 218421 |
| (1S,2R,5S)-(+)-MENTHOL | 15356704 | 224464 |
| (1S,2S,5R)-(+)-NEOMENTHOL | 2216526 | 235180 |
| (1S,2R,5R)-(+)-ISOMENTHOL | 23283978 | 242195 |
| (+/−)-3-CYCLOHEXENE-1-METHANOL | 72581329 | 162167 |
| (+)-P-MENTH-1-EN-9-OL | 13835308 | 183741 |
| (S)-(−)-PERILLYL ALCOHOL | 536594 | 218391 |
| TERPINEN-4-OL | 562743 | 218383 |
| ALPHA-TERPINEOL | 98555 | 218375 |
| (+/−)-TRANS-P-MENTH-6-ENE-2,8-DIOL | 32226543 | 247774 |
| CYCLOHEPTANEMETHANOL | 4448753 | 138657 |
| TETRAHYDROFURFURYL ALCOHOL | 97994 | 185396 |
| (S)-(+)-2-PYRROLIDINEMETHANOL | 23356969 | 186511 |
| 1-METHYL-2-PYRROLIDINEETHANOL | 67004642 | 139513 |
| 1-ETHYL-4-HYDROXYPIPERIDINE | 3518830 | 224634 |
| 3-HYDROXYPIPERIDINE HYDROCHLORIDE | 64051792 | 174416 |
| (+/−)-2-PIPERIDINEMETHANOL | 3433372 | 155225 |
| 3-PIPERIDINEMETHANOL | 4606659 | 155233 |
| 1-METHYL-2-PIPERIDINEMETHANOL | 20845345 | 155241 |
| 1-METHYL-3-PIPERIDINEMETHANOL | 7583531 | 146145 |
| 2-PIPERIDINEETHANOL | 1484840 | 131520 |
| 4-HYDROXYPIPERIDINE | 5382161 | 128775 |
| 4-METHYL-1-PIPERAZINEPROPANOL | 5317339 | 238716 |
| EXO-NORBORNEOL | 497370 | 179590 |
| ENDO-NORBORNEOL | 497369 | 186457 |
| 5-NORBORNENE-2-METHANOL | 95125 | 248533 |
| (+/−)-3-METHYL-2-NORBORNANEMETHANOL | 6968758 | 130575 |
| ((1S)-ENDO)-(−)-BORNEOL | 464459 | 139114 |
| (1R)-ENDO-(+)-FENCHYL ALCOHOL | 2217029 | 196444 |
| 9-ETHYLBICYCLO(3.3.1)NONAN-9-OL | 21951333 | 193895 |
| (+/−)-ISOPINOCAMPHEOL | 51152115 | 183229 |
| (S)-CIS-VERBENOL | 18881044 | 247065 |
| (1R,2R,3R,5S)-(−)-ISOPINOCAMPHEOL | 25465650 | 221902 |
| (1R)-(−)-MYRTENOL | 515004 | 188417 |
| 1-ADAMANTANOL | 768956 | 130346 |
| 3,5-DIMETHYL-1-ADAMANTANOL | 707379 | 231290 |
| 2-ADAMANTANOL | 700572 | 153826 |
| 1-ADAMANTANEMETHANOL | 770718 | 184209 |
| 1-ADAMANTANEETHANOL | 6240115 | 188115 |
| 3-FURANMETHANOL | 4412913 | 196398 |
| FURFURYL ALCOHOL | 98000 | 185930 |
| 2-(3-THIENYL)ETHANOL | 13781674 | 228796 |
| 4-METHYL-5-IMIDAZOLEMETHANOL | 38585625 | 227420 |
| HYDROCHLORIDE | | |
| METRONIDAZOLE | 443481 | 226742 |
| 4-(HYDROXYMETHYL)IMIDAZOLE | 32673419 | 219908 |
| HYDROCHLORIDE | | |
| 4-METHYL-5-THIAZOLEETHANOL | 137008 | 190675 |
| 2-(2-HYDROXYETHYL)PYRIDINE | 103742 | 128643 |
| 2-HYDROXY-6-METHYLPYRIDINE | 3279763 | 128740 |
| 4-PYRIDYLCARBINOL | 586958 | 151629 |
| 3-PYRIDYLCARBINOL N-OXIDE | 6968725 | 184446 |
| 1-BENZYL-4-HYDROXYPIPERIDINE | 4727724 | 152986 |
| 1-(4-CHLOROPHENYL)-1- | 80866791 | 188697 |
| CYCLOPENTANEMETHANOL | | |
| (4S,5S)-(−)-2-METHYL-5-PHENYL-2- | 53732415 | 187666 |
| OXAZOLINE-4-METHANOL | | |
| 6-(4-CHLOROPHENYL)-4,5-DIHYDRO-2-(2- | 38958826 | 243728 |
| HYDROXYBUTYL)-3(2H)-PYRIDAZINONE | | |
| N-(2-HYDROXYETHYL)PHTHALIMIDE | 3891074 | 138339 |
| 2-NAPHTHALENEETHANOL | 1485070 | 188107 |
| 1-NAPHTHALENEETHANOL | 773999 | 183458 |
| 2-ISOPROPYLPHENOL | 88697 | 129526 |
| 4-CHLORO-ALPHA,ALPHA- | 5468973 | 130559 |
| DIMETHYLPHENETHYL ALCOHOL | | |
| 4-FLUORO-ALPHA-METHYLBENZYL | 403418 | 132705 |
| ALCOHOL | | |
| 3-PHENYL-1-PROPANOL | 122974 | 140856 |
| 3-(4-METHOXYPHENYL)-1-PROPANOL | 5406188 | 142328 |
| 4-FLUOROPHENETHYL ALCOHOL | 7589277 | 154172 |
| 4-METHOXYPHENETHYL ALCOHOL | 702238 | 154180 |
| TRANS-2-METHYL-3-PHENYL-2-PROPEN-1-OL | 1504558 | 155888 |
| 2-ANILINOETHANOL | 122985 | 156876 |
| 3-FLUOROBENZYL ALCOHOL | 456473 | 162507 |
| 2-FLUOROBENZYL ALCOHOL | 446515 | 162515 |
| 2-METHYL-1-PHENYL-2-PROPANOL | 100867 | 170275 |
| ALPHA-(CHLOROMETHYL)-2,4- | 13692143 | 178403 |
| DICHLOROBENZYL ALCOHOL | | |
| 2-PHENYL-1-PROPANOL | 1123859 | 179817 |
| 4-CHLOROPHENETHYL ALCOHOL | 1875883 | 183423 |
| 4-BROMOPHENETHYL ALCOHOL | 4654391 | 183431 |
| 4-NITROPHENETHYL ALCOHOL | 100276 | 183466 |
| 2-NITROPHENETHYL ALCOHOL | 15121843 | 183474 |
| BETA-ETHYLPHENETHYL ALCOHOL | 2035941 | 183482 |
| 4-PHENYL-1-BUTANOL | 3360416 | 184756 |
| 2-METHOXYPHENETHYL ALCOHOL | 7417187 | 187925 |
| 3-METHOXYPHENETHYL ALCOHOL | 5020417 | 187933 |
| 3-PHENYL-1-BUTANOL | 2722363 | 187976 |
| 2-METHYLPHENETHYL ALCOHOL | 19819988 | 188123 |
| 3-METHYLPHENETHYL ALCOHOL | 1875894 | 188131 |
| 4-METHYLPHENETHYL ALCOHOL | 699025 | 188158 |
| 5-PHENYL-1-PENTANOL | 10521912 | 188220 |
| 4-(4-METHOXYPHENYL)-1-BUTANOL | 22135508 | 188239 |
| 4-(4-NITROPHENYL)-1-BUTANOL | 79524202 | 188751 |
| 3,3-DIPHENYL-1-PROPANOL | 20017678 | 188972 |
| 1-PHENYL-2-PROPANOL | 14898874 | 189235 |
| (+/−)-ALPHA-ETHYLPHENETHYL ALCOHOL | 701702 | 190136 |
| 1,1-DIPHENYL-2-PROPANOL | 29338496 | 190756 |
| 3-CHLOROPHENETHYL ALCOHOL | 5182445 | 193518 |
| 2-CHLOROPHENETHYL ALCOHOL | 19819955 | 193844 |
| (+/−)-1-PHENYL-2-PENTANOL | 705737 | 195286 |
| 2,2-DIPHENYLETHANOL | 1883325 | 196568 |
| 4-ETHOXY-3-METHOXYPHENETHYL | 77891293 | 197599 |
| ALCOHOL | | |
| 3,4-DIMETHOXYPHENETHYL ALCOHOL | 7417212 | 197653 |
| 3-(3,4-DIMETHOXYPHENYL)-1-PROPANOL | 3929473 | 197688 |
| 2-(4-BROMOPHENOXY)ETHANOL | 34743889 | 198765 |
| 2-FLUOROPHENETHYL ALCOHOL | 50919067 | 228788 |
| 3-(TRIFLUOROMETHYL)PHENETHYL | 455016 | 230359 |
| ALCOHOL | | |
| 2-(PHENYLTHIO)ETHANOL | 699127 | 232777 |
| 1-(2-METHOXYPHENYL)-2-PROPANOL | 15541261 | 233773 |

TABLE 27
Exemplary Method Embodiments of Processes A-R

A; B; C; D; I; J; K; L; M; N; O; P; Q; R; E; F; G; H;
AB; BC; CD; DI; IJ; JK; KL; LM; MN; NO; OP;
OQ; QR; EF; FG; GH; HI; ABC; BCD; CDI; DIJ;
IJK; JKL; KLM; LMN; MNO; NOP; NOQ; OQR; EFG;
FGH; GHI; HIJ; ABDC; BCDI; CDIJ; DIJK; IJKL;
JKLM; KLMN; LMNO; MNOP; MNOQ; NOQR; EFHG;
FGHI; GHIJ; HIJK; ABCDI; BCDIJ; CDIJK; DIJKL; IJKLM;
JKLMN; KLMNO; LMNOP; LMNOQ; MNOQR; EFGHI;
FGHIJ; GHIJK; HIJKL; ABCDIJ; BCDIJK; CDIJKL; DIJKLM;
IJKLMN; JKLMNO; KLMNOP; KLMNOQ; LMNOQR;
EFGHIJ; FGHIJK; GHIJKL; HIJKLM; ABCDIJK; BCDIJKL;
CDIJKLM; DIJKLMN; IJKLMNO; JKLMNOP; JKLMNOQ;
KLMNOQR; EFGHIJK; FGHIJKL; GHIJKLM; HIJKLMN;
ABCDIJKL; BCDIJKLM; CDIJKLMN; DIJKLMNO; IJKLMNOP;
IJKLMNOQ; JKLMNOQR; EFGHIJKL; FGHIJKLM;
GHIJKLMN; HIJKLMNO; ABCDIJKLM; BCDIJKLMN;
CDIJKLMNO; DIJKLMNOP; DIJKLMNOQ; IJKLMNOQR;
EFGHIJKLM; FGHIJKLMN; GHIJKLMNO; HIJKLMNOP;
HIJKLMNOQ; ABCDIJKLMN; BCDIJKLMNO; CDIJKLMNOP;
CDIJKLMNOQ; DIJKLMNOQR; EFGHIJKLMN;
FGHIJKLMNO; GHIJKLMNOP; GHIJKLMNOQ; HIJKLMNOQR;
ABCDIJKLMNO; BCDIJKLMNOP; BCDIJKLMNOQ;
CDIJKLMNOQR; EFGHIJKLMNO; FGHIJKLMNOP;
FGHIJKLMNOQ; GHIJKLMNOQR; ABCDIJKLMNOP;
ABCDIJKLMNOQ; BCDIJKLMNOQR; EFGHIJKLMNOP;
EFGHIJKLMNOQ; FGHIJKLMNOQR; ABCDIJKLMNOQR;
EFGHIJKLMNOQR; S; T; U; V; W; ST; TU; UV; VW; STU; TUV;
UVW; STUV; TUVW; STUVW.

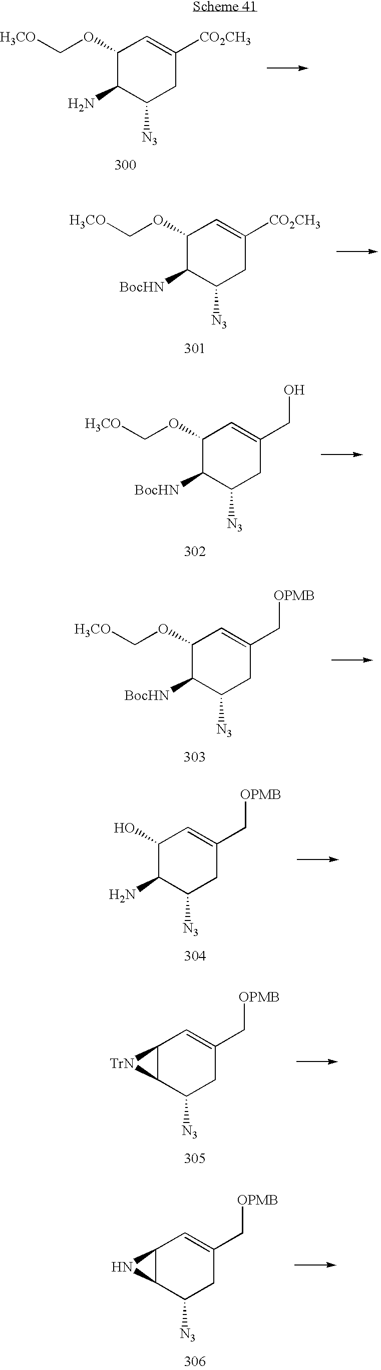

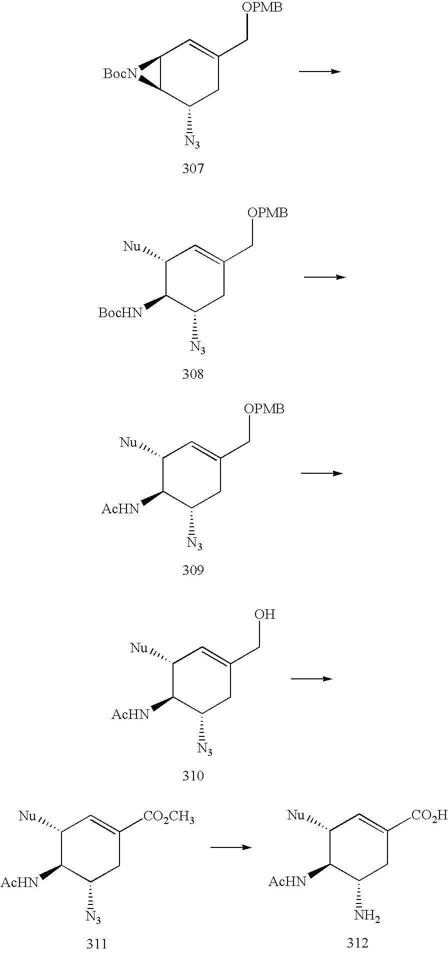

Scheme 41

The amine 300 (an intermediate in Example 52, optionally purified prior to use) is treated with Boc anhydride to give the mono Boc protected amine 301. Such a transformation is found in Greene, T. W. “Protective Groups in Organic Synthesis” 2nd Ed. cohn Wiley & Sons, New York, 1991) pages 327-328.

Methyl ester 301 is reduced to the corresponding primary allylic alcohol 302 with DIBAL at low temperature. Such a conversion is described by Garner, P. and Park, J. M., “J. Org. Chem.”, 52:2361 (1987).

The primary alcohol 302 is protected as its p-methoxy benzyl ether derivative 303 by treatment with 4-methoxybenzyl chloride under basic conditions. Such a conversion is described in Horita, K. et. al., “Tetrahedron”, 42:3021 (1986).

The MOM and Boc protecting groups of 303 are removed by treatment with TFA/CH₂Cl₂ to give the amino alcohol 304. Such transformations are found in Greene, T. W. “Protective Groups in Organic Synthesis”, 2nd. Ed. (John Wiley & Sons, New York, 1991).

Conversion of 304 into the corresponding trityl protected aziridine 305 is accomplished in a one pot reaction two step sequence: 1) TrCl/TEA, 2) MsCl/TEA. Such a transformation has been previously described.

Aziridine 305 is then converted the corresponding Boc protected derivative 307 by first removal of the trityl group with HCl/acetone to give 306. Such a transformation is described in Hanson, R. W. and Law, H. D. “J. Chem. Soc.”, 7285 (1965). Aziridine 306 is then converted into the corresponding Boc derivative 307 by treatment with Boc anhydride. Such a conversion is described in Fitremann, J., et. al. “Tetrahedron Lett.”, 35:1201 (1994).

The allylic aziridine 307 is opened selectively at the allylic position with a higher order organocuprate in the presence of BF₃.Et₂O at low temperature to give the opened adduct 308. Such an opening is described in Hudlicky, T., et. al. “Synlett.” 1125 (1995).

The Boc protected amine 308 is converted into the N-acetyl derivative 309 in a two step sequence: 1) TFA/CH₂Cl₂; 2) Ac₂O/pyridine. Such transformations can be found in Greene, T. W., “Protective Groups in Organic Synthesis”, 2nd. Ed. (John Wiley & Sons, New York, 1991) pages 327-328 and pages 351-352.

Benzyl ether 309 is deprotected with DDQ at room temperature to give the primary allylic alcohol 310. Such a transformation is found in Horita, K., et. al. “Tetrahedron” 42:3021 (1986).

Alcohol 310 is oxidized and converted in a one pot reaction into the methyl ester 311 via a Corey oxidation using MnO2/AcOH/MeOH/NaCN. Such a transformation can be found in Corey, E. J., et. al. “J. Am. Chem. Soc.”, 90:5616 (1968).

Azido ester 311 is converted into amino acid 312 in a two step sequence 1) Ph₃P/H₂O/THF; 2) KOH/THF. Such a conversion has been described previously.

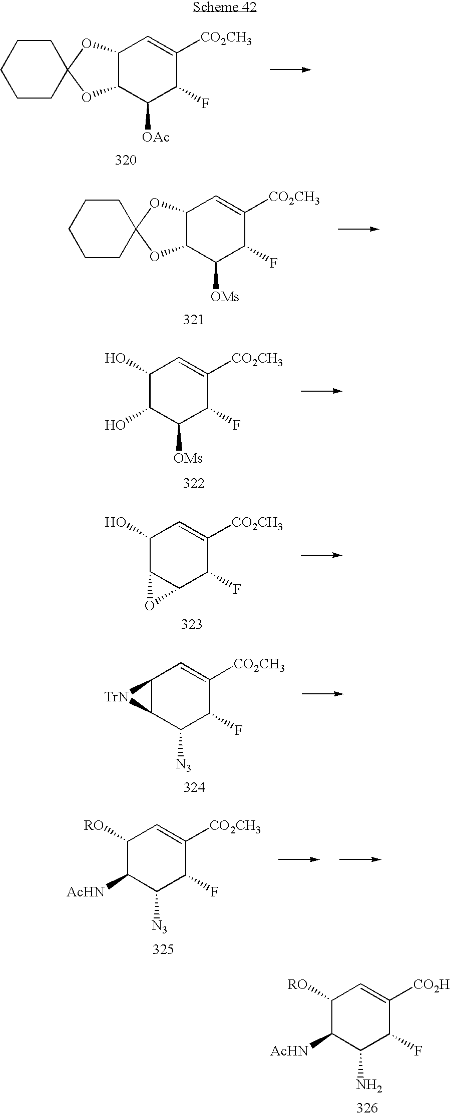

Scheme 42

The known fluoro acetate 320 (Sutherland, J. K., et.al. “J. Chem. Soc. Chem. Commun.” 464 (1993) is deprotected to the free alcohol and then converted into the corresponding mesylate 321 in two steps: 1) NaOMe; 2) MsCl/TEA. Such transformations are described in Greene, T. W., “Protective Groups in Organic Synthesis”, 2nd. Ed. (John Wiley & Sons, New York, 1991).

Deprotection of 321 under acidic conditions gives diol 322 which is cyclized to the epoxy alcohol 323 under basic conditions. Such a conversion has been previously described.

Conversion of 323 to the N-trityl protected aziridine 324 is accomplished with the following sequence: 1) MOMCl/TEA; 2) NaN₃/NH₄Cl; 3) MsCl/TEA; 4) PPh₃/TEA/H₂O; 5) NaN₃/NH₄Cl; 6) HCl/MeOH; 7) i)TrCl, ii) MsCl/TEA. Such a sequence has been previously described.

The aziridine 324 is then opened with the appropriate alcohol under Lewis acid conditions and then treated with Ac₂O/pyridine to give the acetylated product 325. Such a transformation has been previously described.

The ester 325 is converted to the corresponding amino acid 326 in a two step sequence: 1) PPh₃/H₂O/THF; 2) KOH/THF. Such a transformation has been previously described.

U.S. Pat. No. 5,214,165, and in particular, the “Descriptions and Examples” at column 9, line 61 to column 18, line 26, describes the preparation of 6α and 6β fluoro Shikimic acid (numbering is as described therein). These fluoro compounds are suitable starting materials for methods of making compounds of the invention that use Shikimic acid.

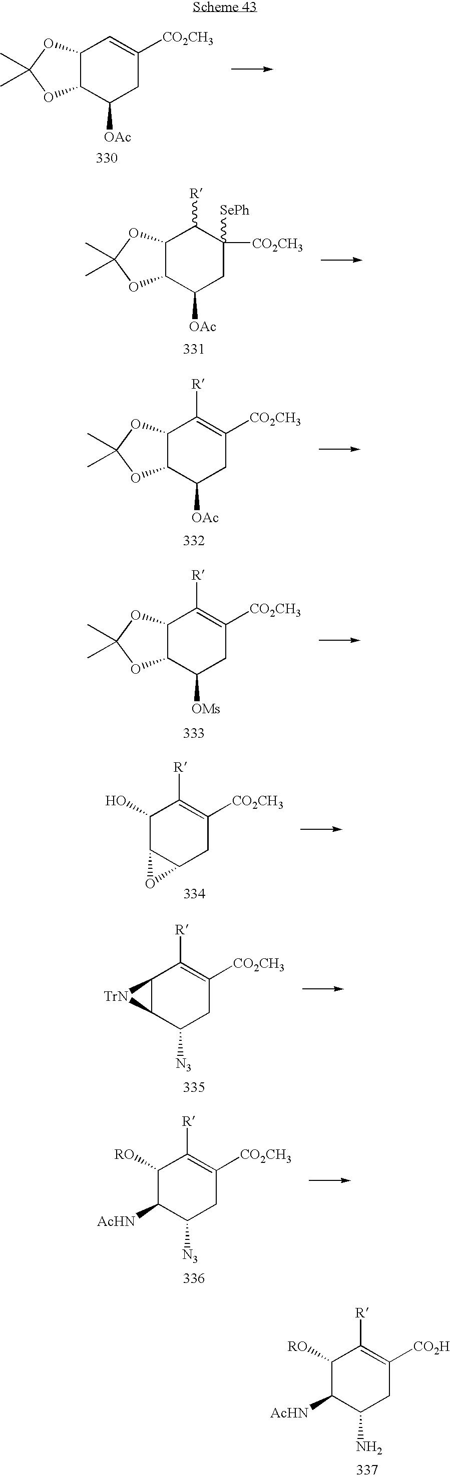

Scheme 43

Unsaturated ester 330 (obtainable by standard actetylation methods from the acetonide alcohol described in Campbell, M. M., et. al., “Synthesis”, 179 (1993)) is reacted with the appropriate organocuprate where R′ is the ligand to be transferred from the organocuprate (R′ is J₁ₐ). The resultant intermediate is then trapped with PhSeCl to give 331 which is then treated with 30% H₂O₂ to give the α:β-unsaturated ester 332. Such a transformation can be found in Hayashi, Y., et. al, “J. Org. Chem.” 47:3428 (1982).

Acetate 332 is then converted into the corresponding mesylate 333 in a two step sequence: 1) NaOMe/MeOH; 2) MsCl/TEA. Such a transformation has been previously described and can also be found in Greene, T. W., “Protective Groups in Organic Synthesis”, 2nd. Ed. (John Wiley & Sons, New York, 1991).

The acetonide 333 is then converted into the epoxy alcohol 334 in a two step sequence: 1) p-TsOH/MeOH/.; 2) DBU/THF. Such a transformation has been previously described.

Conversion of epoxide 334 into N-trityl aziridine 335 is accomplished by the following sequence: 1) MOMCl/TEA; 2) NaN₃/NH₄Cl; 3) MsCl/TEA; 4) PPh₃/TEA/H₂O; 5) NaN₃/NH₄Cl; 6) HCl/MeOH; 7) i)TrCl, ii) MsCl/TEA. Such a sequence has been previously described.

The aziridine 335 is then opened with the appropriate alcohol under Lewis acid conditions and then treated with Ac₂O/pyridine to give the acetylated product 336. Such a transformation has been previously described.

The azido ester 336 is converted to the corresponding amino acid 337 in a two step sequence: 1) PPh₃/H₂O/THF; 2) KOH/THF. Such a transformation has been previously described.

Schemes 44 and 45 are referred to in the examples.

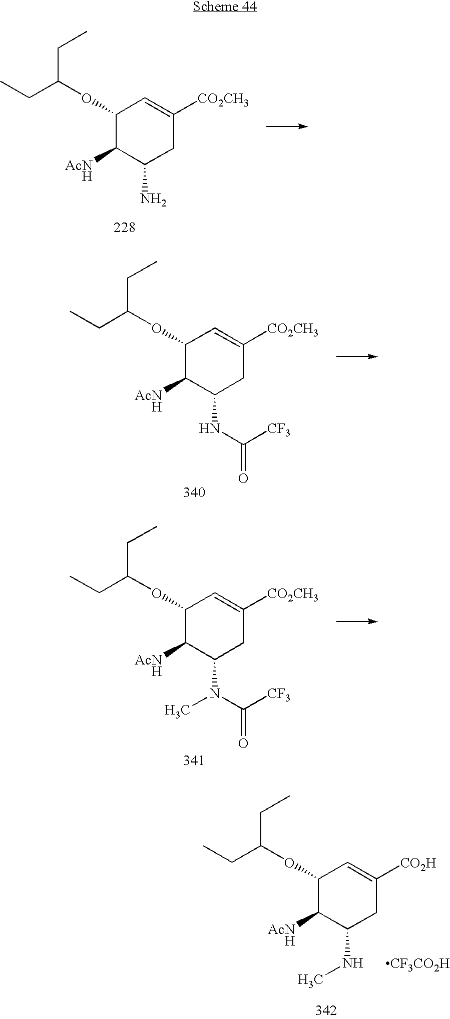

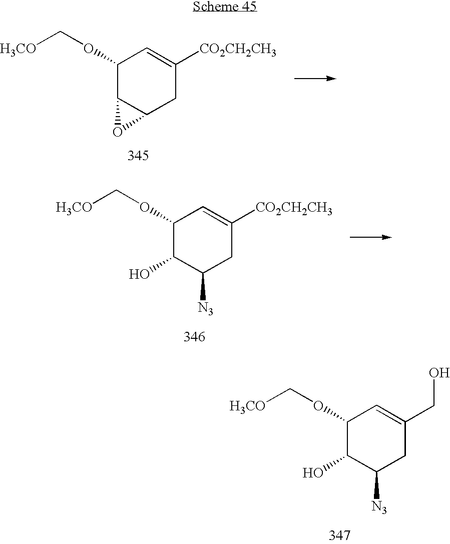

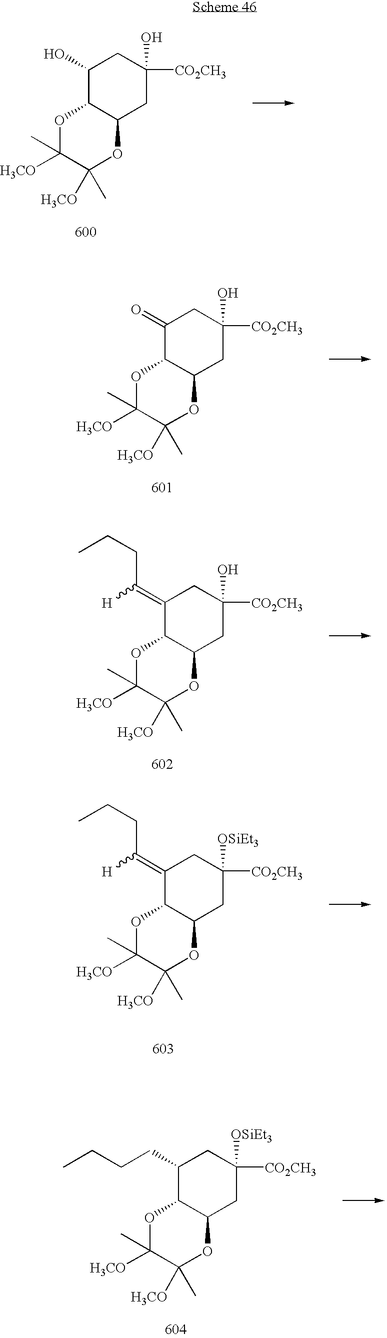

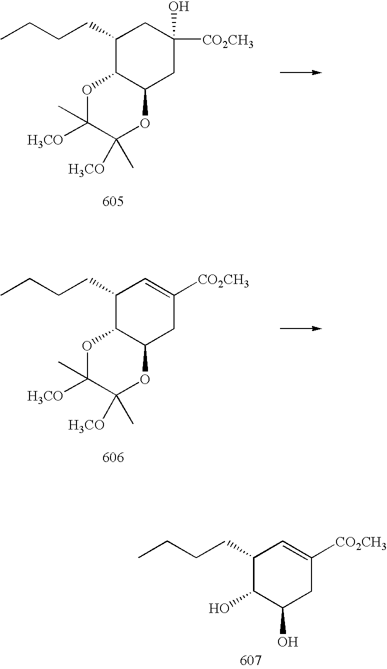

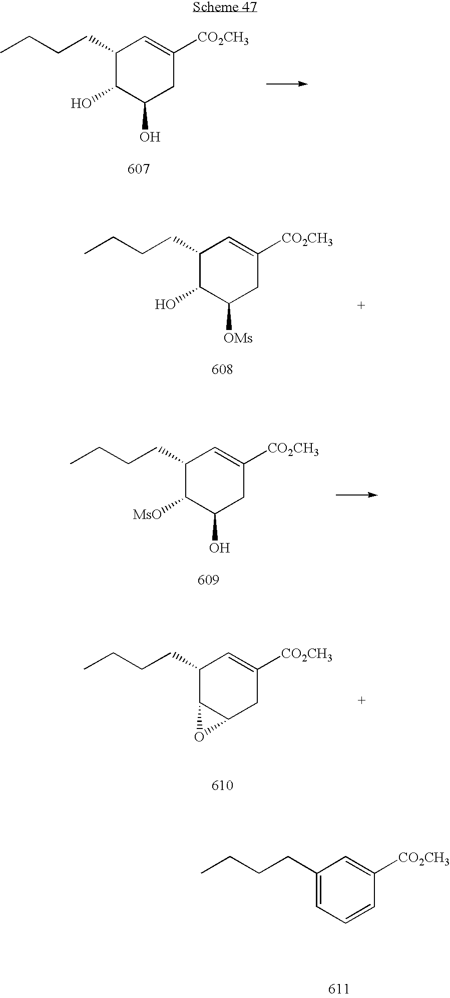

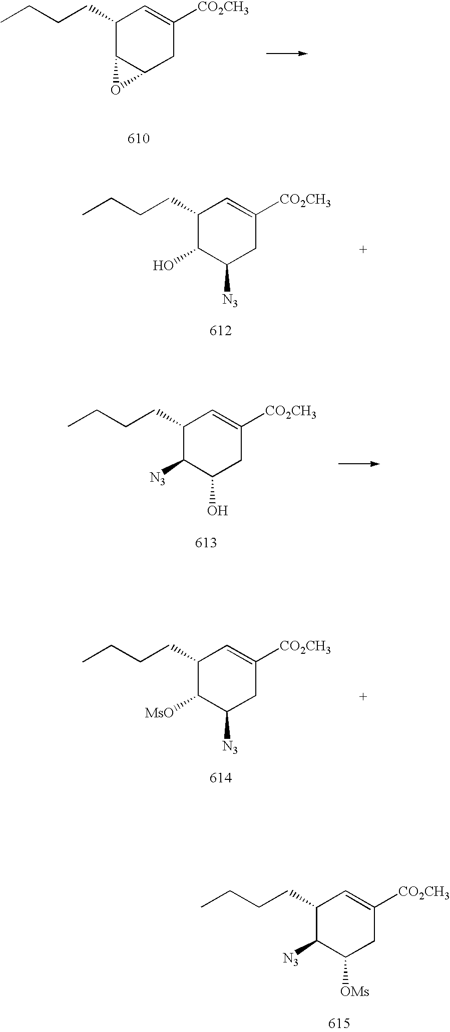

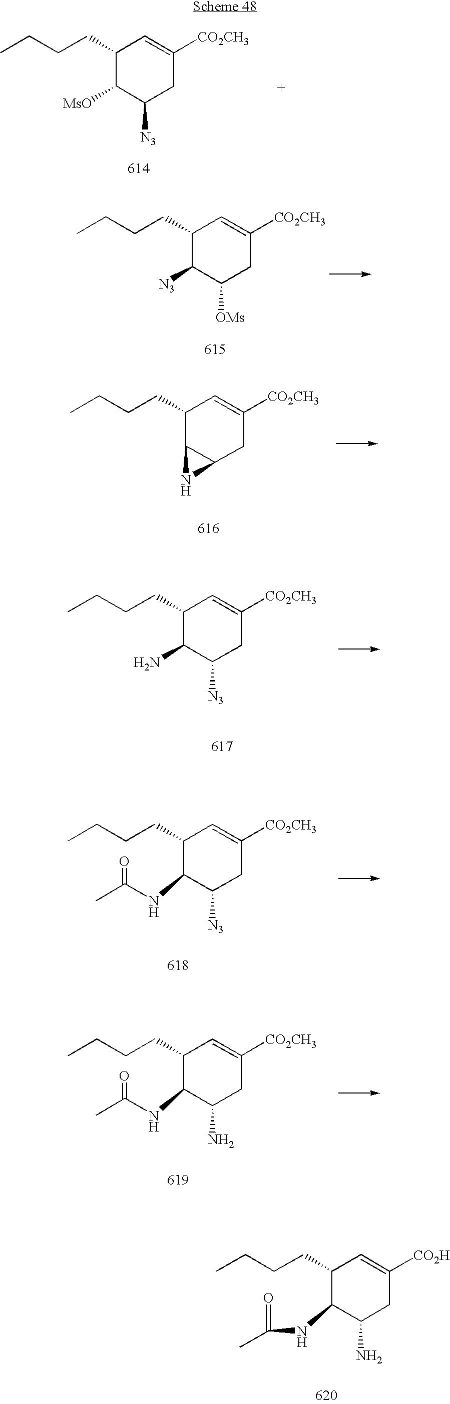

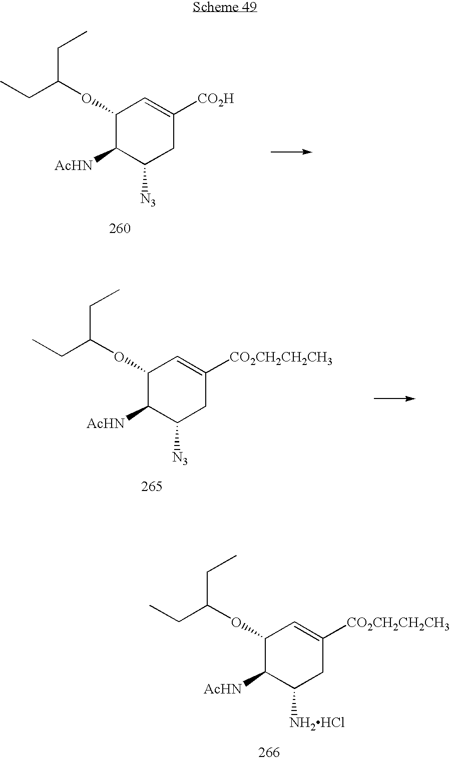

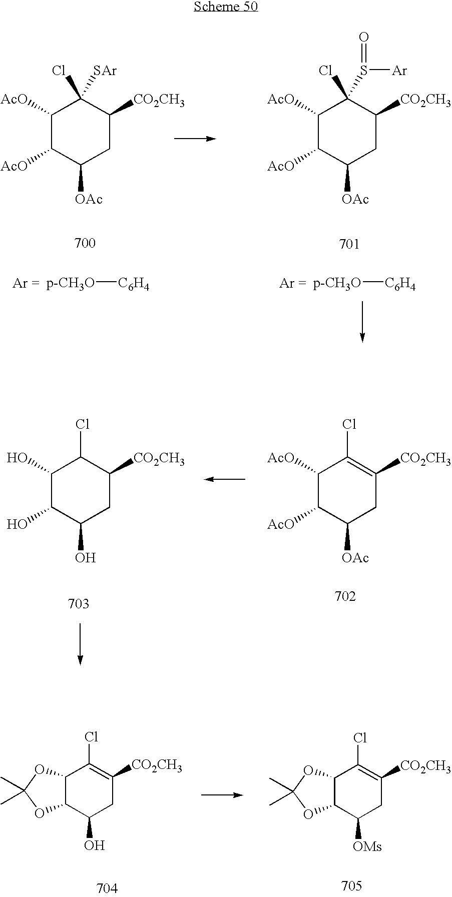

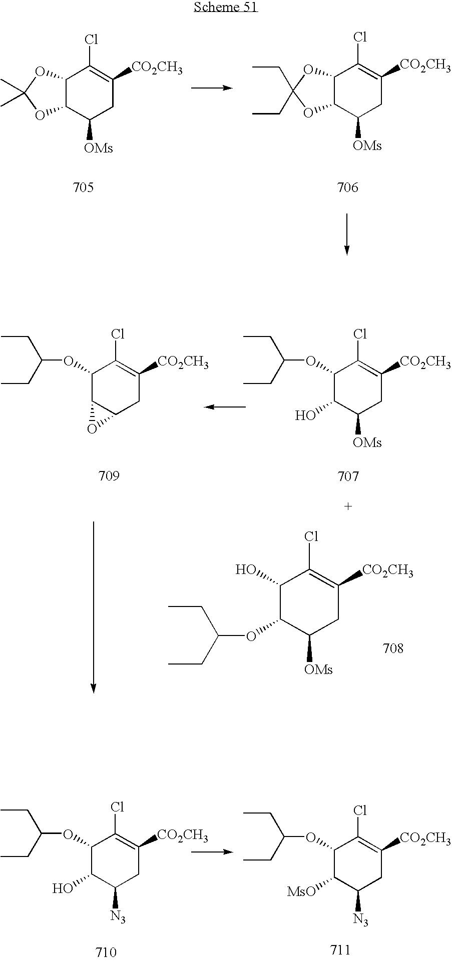

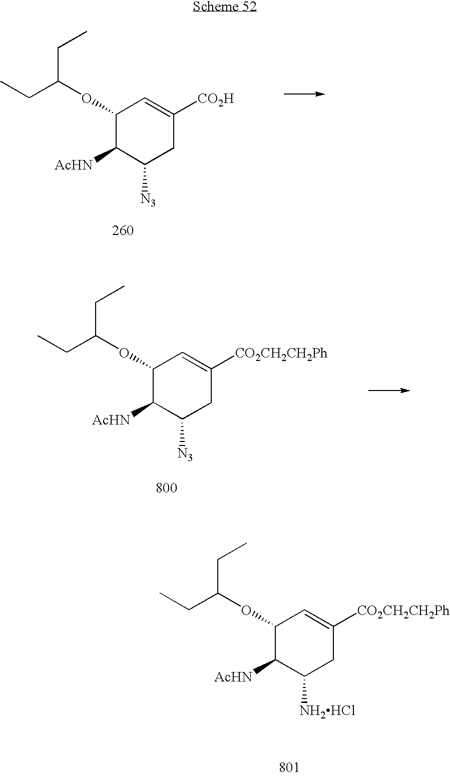

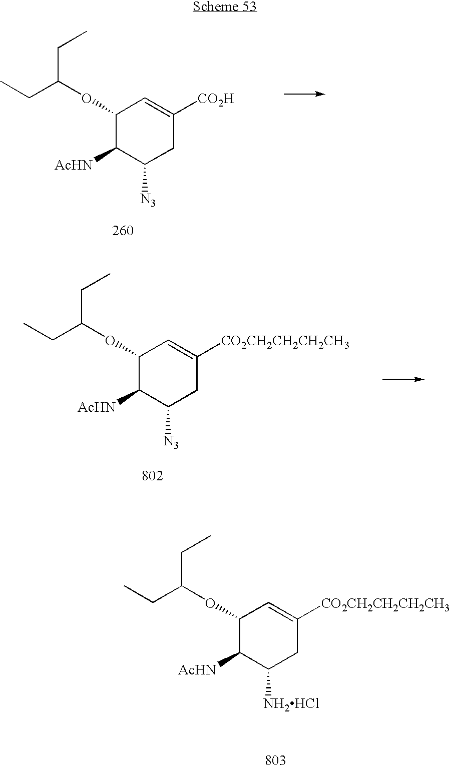

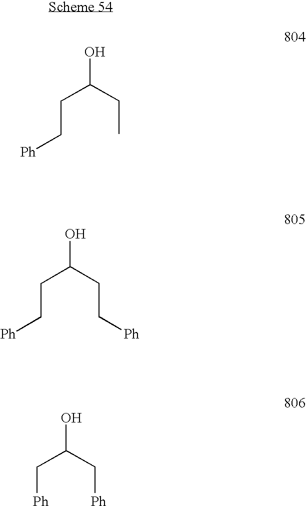

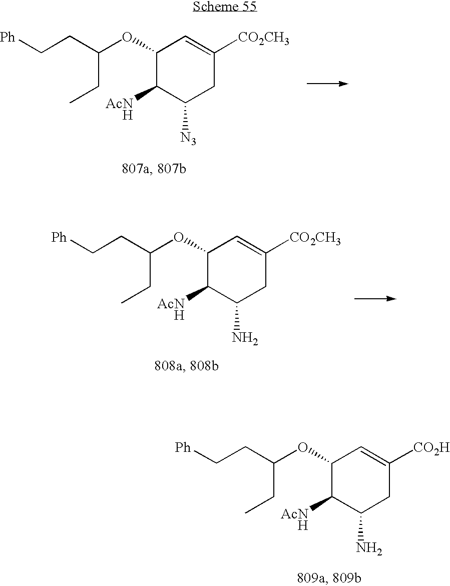

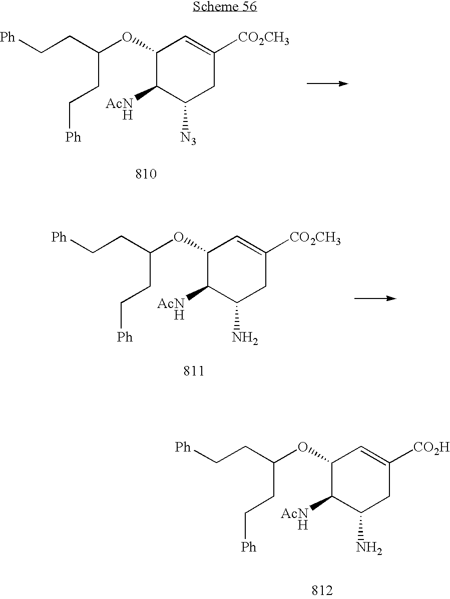

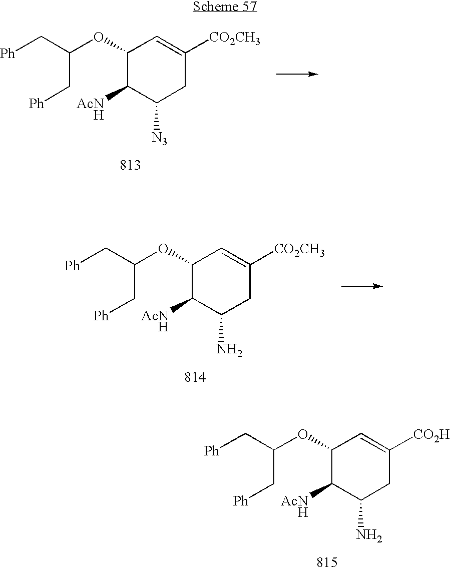

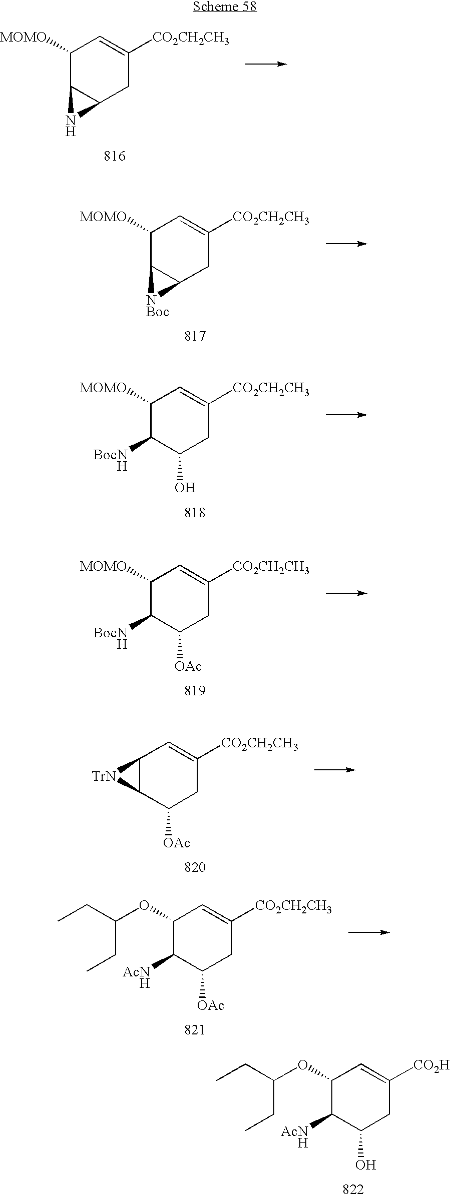

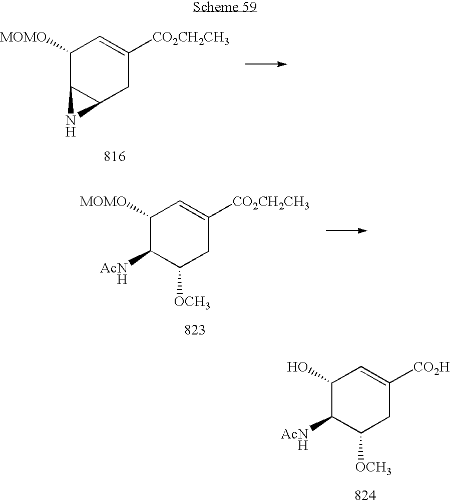

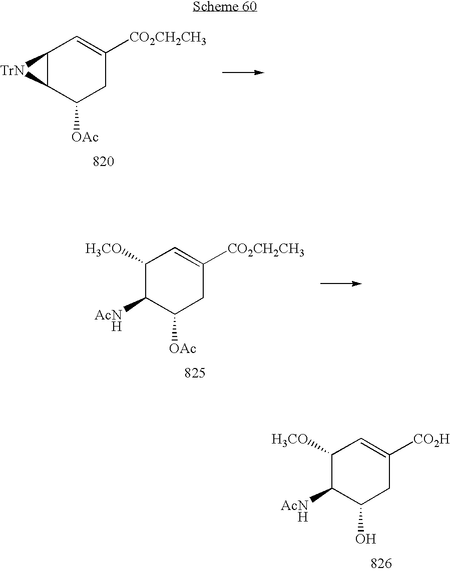

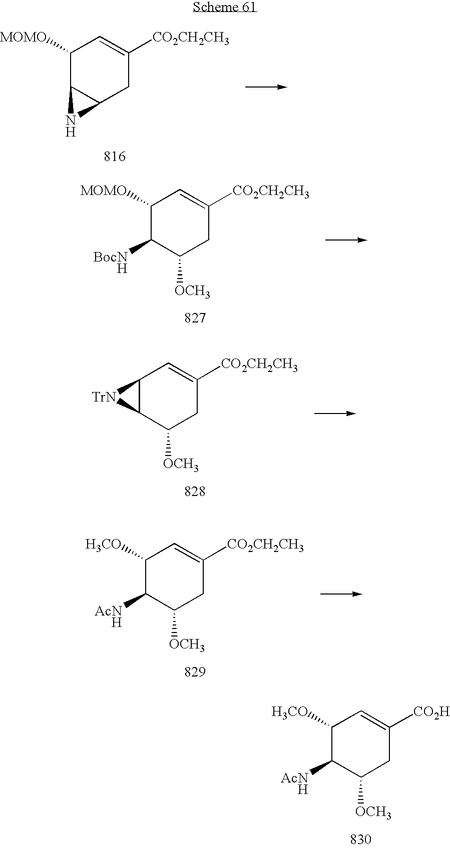

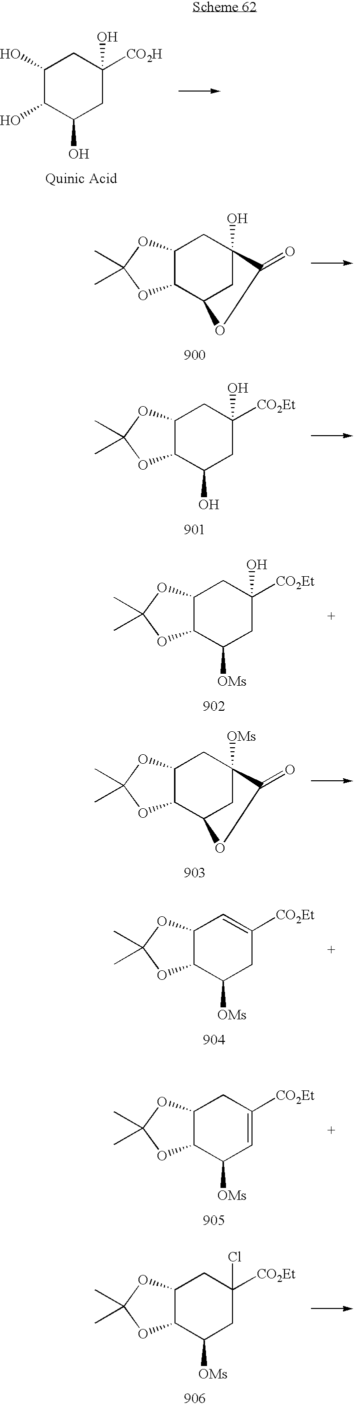

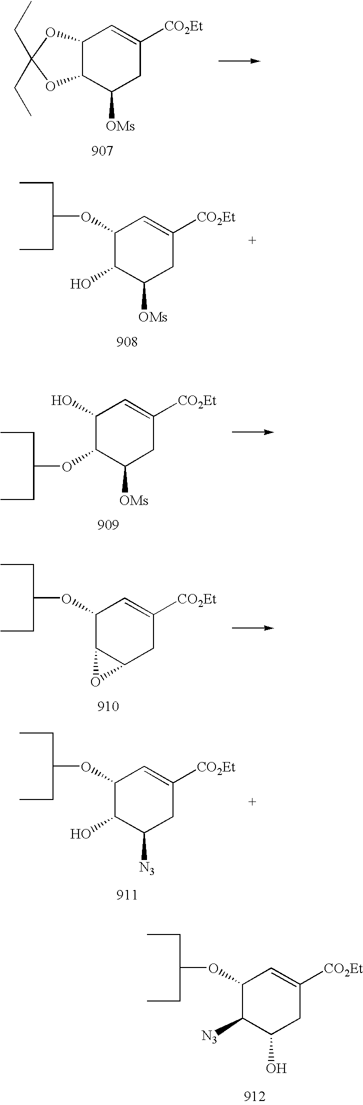

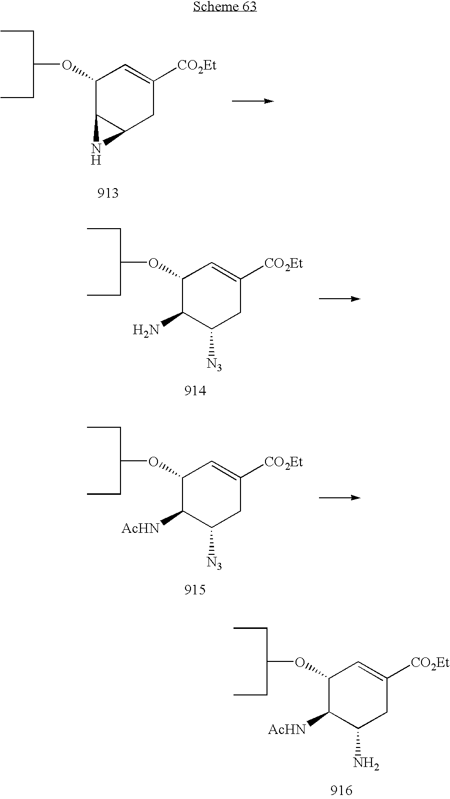

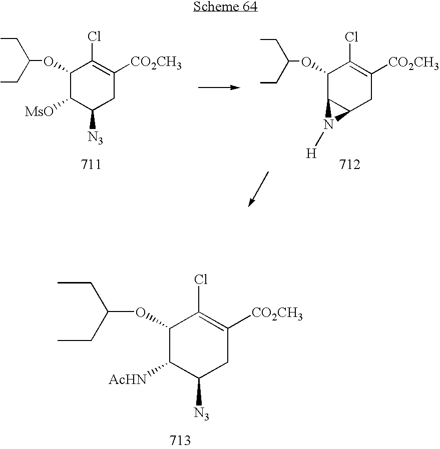

Modification of the exemplary starting materials to form different E₁ groups has been described in detail and will not be elaborated here. See Fleet, G. W. J. et al.; “J. Chem. Soc. Perkin Trans. I”, 905-908 (1984), Fleet, G. W. J. et al.; “J. Chem. Soc., Chem. Commun.”, 849-850 (1983), Yee, Ying K. et al.; “J. Med. Chem.”, 33:2437-2451 (1990); Olson, R. E. et al.; “Bioorganic & Medicinal Chemistry Letters”, 4(18):2229-2234 (1994); Santella, J. B. III et al.; “Bioorganic & Medicinal Chemistry Letters”, 4(18):2235-2240 (1994); Judd, D. B. et al.; “J. Med. Chem.”, 37:3108-3120 (1994) and Lombaert, S. De et al.; “Bioorganic & Medicinal Chemistry Letters”, 5(2):151-154 (1994).

The E₁ sulfur analogs of the carboxylic acid compounds of the invention are prepared by any of the standard techniques. By way of example and not limitation, the carboxylic acids are reduced to the alcohols by standard methods. The alcohols are converted to halides or sulfonic acid esters by standard methods and the resulting compounds are reacted with NaSH to produce the sulfide product. Such reactions are described in Patai, “The Chemistry of the Thiol Group” (John Wiley, New York, 1974), pt. 2, and in particular pages 721-735.

Modifications of each of the above schemes leads to various analogs of the specific exemplary materials produced above. The above cited citations describing suitable methods of organic synthesis are applicable to such modifications.

In each of the above exemplary schemes it may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example, size exclusion or ion exchange chromatography, high, medium, or low pressure liquid chromatography, small scale and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example, boiling point, and molecular weight in distillation and sublimation, presence or absence of polar functional groups in chromatography, stability of materials in acidic and basic media in multiphase extraction, and the like. One skilled in the art will apply techniques most likely to achieve the desired separation.

All literature and patent citations above are hereby expressly incorporated by reference at the locations of their citation. Specifically cited sections or pages of the above cited works are incorporated by reference with specificity. The invention has been described in detail sufficient to allow one of ordinary skill in the art to make and use the subject matter of the following claims. It is apparent that certain modifications of the methods and compositions of the following claims can be made within the scope and spirit of the invention.

Enteric Protection

Another embodiment of the present invention is directed toward enteric protected forms of the compounds of the invention. As used herein the term “enteric protection” means protecting a compound of the invention in order to avoid exposing a portion of the gastrointestinal tract, typically the upper gastrointestinal tract, in particular the stomach and esophagus, to the compound of this invention. In this way gastric mucosal tissue is protected against rates of exposure to a compound of the invention which produce adverse effects such as nausea; and, alternatively, a compound of the invention is protected from conditions present in one or more portions of the gastrointestinal tract, typically the upper gastrointestinal tract.

By way of example and not limitation, such enterically protected forms include enteric coated vehicles, such as enteric coated tablets, enteric coated granules, enteric coated beads, enteric coated particles, enteric coated microparticles, and enteric coated capsules. In preferred embodiments, a compound of the invention is placed in a suitable vehicle such as a tablet, granule or capsule, and the vehicle is coated with a pharmaceutically acceptable enteric coating. In alternative preferred embodiments, a compound of the invention is prepared as enterically protected granules, particles, microparticles, spheres, microspheres, or colloids, and the enteric protected granules, particles, microparticles, spheres, microspheres, or colloids, are prepared as pharmaceutically acceptable dosage forms such as tablets, granules, capsules, or suspensions.

One aspect of the invention is directed to enteric-coated dosage forms of the compounds of the invention to effect delivery to the intestine of a human or other mammal, preferably to the small intestine, of a pharmaceutical composition comprised of a therapeutically effective amount of about 0.1-1000 mg of an active ingredient and optional pharmaceutically acceptable excipients.

The term “vehicle” as used herein includes pharmaceutically acceptable dose vehicles. Many vehicles are well known in the art cited herein such as tablet, coated tablet, capsule, hard capsule, soft gelatin capsule, particle, microparticle, sphere, microsphere, colloid, microencapsulationed, sustained release, semisolid, suppository or granule vehicles.

The term “pharmaceutically-acceptable excipients” as used herein includes any physiologically inert, pharmacologically inactive material known to one skilled in the art, which is compatible with the physical and chemical characteristics of the particular compound of the invention selected for use. These excipients are described elsewhere herein. The excipients may, but need not, provide enteric protection.

The term “unit dose” is used herein in the conventional sense to mean a single application or administration of the compound of this invention to the subject being treated in an amount as stated below. It should be understood that a therapeutic or prophylactic dosage can be given in one unit dose, or alternatively, in multiples of two or more of such dose units with the total adding up to the desired amount of compound for a given time period.

In general, the oral unit dosage form compositions of this invention, preferably employ from about 1 to about 1000 milligrams (mg), typically, about 10 to 500 mg, more typically from about 50 to about 300 mg, more typically yet, 75 mg of the compound for each unit dose. The actual amount will vary depending upon the active compound selected.

In typical embodiments, an enteric protectant is applied to the vehicle containing the compound, or to the compound without vehicle, the protectant prevents nausea inducing exposure, contact or rates of exposure of the mouth, esophagus or stomach with the compound, but which releases the compound for absorption when the dosage form passes into the proximal portion of the lower gastrointestinal tract, or in some embodiments, substantially only in the colon.

The relative proportions of the protectant and compound of the invention are varied to achieve optimum absorption depending on the compound selected. The minimum or maximum amount of enteric protectant by weight percent is not critical. Typically, enteric protected embodiments contain less than about 50% enteric coating by weight. More typically about 1% to about 25%, still more typically, about 1% to about 15%, more typically yet, about 1% to about 10% (all by weight).

A number of monographs describe enteric protection and related technology which are useful in preparing the enterically protected compositions of the invention. Such monographs include: “Theory and Practice of Industrial Pharmacy,” 3rd ed. Lea & Febiger, Philadelphia, 1986 (ISBN 0-8121-0977-5); Lehmann, K.; “Practical Course in Laquer Coating,”, Eudragit, 1989; Lieberman; Lachman, L.; Schwartz, “Pharmaceutical Dosage Forms: Tablets”, 1990, Dekker (ISBN: 0-8247-8289-5); Lee, Ping I. Editor Good, William R. Editor, “Controlled-Release Technology: Pharmaceutical Applications”, ACS Symposium Ser. Vol. 348 (ISBN: 0-608-03871-7); Wilson, Billie E.; Shannon, Margret T., “Dosage Calculation: A Simplified Approach”, 1996, Appleton & Lange (ISBN: 0-8385-9297-X); Lieberman, Herbert A. Editor Rieger, Martin M., “Pharmaceutical Dosage Forms—Disperse Systems”, 1996, Dekker (ISBN: 0-8247-9387-0); “Basic Tests for Pharmaceutical Dosage Forms”, 1995, World Health (ISBN: 92-4-154418-X); Karsa, D. R., Editor; Stephenson, R. A., Editor, “Excipients & Delivery Systems for Pharmaceutical Formulations: Proceedings of the “Formulate '94” British Association for Chemical Specialties Symposium”, 1995, CRC Pr (ISBN: 0-85404-715-8); Ansel, Howard C.; Popovich, Nicholas G.; Allen, Lloyd V., “Pharmaceutical Dosage Forms & Drug Delivery Systems, 6th ed.”, 1994, Williams & Wilkins (ISBN: 0-683-01930-9); “The Sourcebook for Innovative Drug Delivery: Manufacturers of Devices & Pharmaceuticals, Suppliers of Products & Services, Sources of Information”, 1987, Canon Comns (ISBN: 0-9618649-0-7); Chiellini, E., Editor; Giusti, G., Editor; Migliaresi, C., Editor; Nicolais, L., Editor, “Polymers in Medicine II: Biomedical & Pharmaceutical Applications”, 1986, Plenum (ISBN: 0-306-42390-1); “Pharmaceutical Aerosol: A Drug Delivery System in Transition”, 1994, Technomic (ISBN: 0-87762-971-4); Avis; Lieberman, L.; Lachman, “Pharmaceutical Dosage Forms: Parenteral Medication, 2nd Expanded; Revised ed.”, 1992, Dekker (ISBN: 0-8247-9020-0); Laffer, U., Editor; Bachmann, I., Editor; Metzger, U., Editor, “Implantable Drug Delivery Systems”, 1991, S Karger (ISBN: 3-8055-5434-6); Borchardt, Ronald T., Editor; Repta, Arnold J., Editor; Stella, Valentino J., Editor, “Directed Drug Delivery: A Multidisciplinary Approach”, 1985, Humana (ISBN: 0-89603-089-X); Anderson, James M., Editor, “Advances in Drug Delivery Systems 5: Proceedings of the Fifth International Symposium on Recent Advances in Drug Delivery Systems, Salt Lake City, Utah., U. S. A., February 25-28, 1991”, Elsevier (ISBN: 0-444-88664-8); Turco, Salvatore J.; King, Robert E., “Sterile Dosage Forms: Their Preparation & Clinical Application”, 1987, Williams & Wilkins (ISBN: 0-8121-1067-6); Tomlinson, E., Editor; Davis, S. S., Editor, “Site-Specific Drug Delivery: Cell Biology, Medical & Pharmaceutical Aspects”, 1986, Wiley (ISBN: 0-471-91236-0); Hess, H., Editor, “Pharmaceutical Dosage Forms & Their Use”, 1986, Hogrefe & Huber Pubs (ISBN: 3-456-81422-4); Avis; Lieberman; Lachman, “Pharmaceutical Dosage Forms, Vol. 2”, 1986, Dekker (ISBN: 0-8247-7085-4); Carstensen, Jens T., “Pharmaceutics of Solids & Solid Dosage Forms”, 1977, Wiley (ISBN: 0-471-13726-X); Robinson, Joseph R., Editor, “Ophthalmic Drug Delivery Systems”, 1980, Am Pharm Assn (ISBN: 0-917330-32-3); Ansel, Howard C., “Introduction to Pharmaceutical Dosage Forms, 4th ed.”, 1985, Williams & Wilkins (ISBN: 0-8121-0956-2); “High Tech Drug Delivery Systems”, 1984, Intl Res Dev (ISBN: 0-88694-622-0); Swarbrick, James, “Current Concepts in Pharmaceutical Sciences: Dosage Form Design & Bioavailability”, 1985, Lea & Febiger (ISBN: 0-318-79917-0); Sprowls, Joseph B., Editor, “Prescription Pharmacy: Dosage Formulation & Pharmaceutical Adjuncts, 2nd ed.”, 1970, Lippincott (ISBN: 0-397-52050-6); and Polderman, J., Editor, “Formulation & Preparation of Dosage Forms: Proceedings of the 37th International Congress of Pharmaceutical Sciences of F.I.P., The Hague, Netherlands, September, 1977”, Elsevier (ISBN: 0-444-80033-6).

Specific Embodiments:

In another embodiment, the inventive composition is in the form of an enteric coated tablet dosage form. In this embodiment, the formulation is formed into a hard tablet by conventional means and the tablet is coated with the enteric coating in accordance with conventional techniques.

In a preferred embodiment, the inventive compound is in the form of an enteric coated powder dosage form. In this embodiment, the formulation is filled into a hard or soft-shell capsule or their equivalent and the capsule is coated with the enteric coating in accordance with conventional techniques.

In one embodiment the inventive composition is in the form of a liquid suspension of enteric coated particles of a compound of the invention. In this embodiment, a suspension of the inhibitor in a liquid is filled into a hard or soft-shell capsule or their equivalent and the capsule is coated with the enteric coating in accordance with conventional techniques.

As alternatives to the foregoing embodiments the capsule or other dosage container is itself constructed of an enteric protection reagent or component, or otherwise is integral to the container.

In another embodiment enteric protectants are used to administer a compound of the invention to the colon. The delivery system is a tablet comprised of three layers: 1) a core containing the active compound of the invention; 2) a non-swelling, erodible polymer layer surrounding the core (with the combination of core and erodible polymer layer being referred to as the “dual matrix tablet”); and 3) an enteric coating applied to the dual matrix tablet. The composition and function of the components of such a colon targeted delivery system are further described in U.S. Pat. No. 5,482,718, which is incorporated herein by reference in its entirety at this location, in particular column 2, line 29, to column 4, line 12, are incorporated herein with specificity.

Another embodiment of the invention is directed toward enteric protected emulsion, suspension, tablet, coated tablet, hard capsule, soft gelatin capsule, microencapsulation, sustained release, liquid, semisolid, suppositorie, and aerosol dosage forms of the compounds of the invention. “Theory and Practice of Industrial Pharmacy,” 3rd ed. Lea & Febiger, Philadelphia, 1986 (ISBN 0-8121-0977-5), describes each of these standard dosage forms in detail at the following locations: emulsion and suspension dosage forms (pp. 100-122), tablets (pp. 293-345), coated tablet (pp. 346-373), hard capsules (pp. 374-397), soft gelatin capsules (pp. 398-411), microencapsulation (pp. 412-430), sustained release dosage forms (pp. 430-456), liquids (pp. 457-478), pharmaceutical suspensions (pp. 479-501), emulsions (pp. 502-533), semisolids (pp. 534-563), suppositories (pp. 564-587), and pharmaceutical aerosols (pp. 589-618).

Alternative embodiments include enteric protected sustained release, controlled release, particulate, microencapsulated, multiparticulate, microparticulate, colloidal, nasal, inhalation, oral mucosal, colonic, dermal, transdermal, ocular, topical, and veterinary dosage forms of the compounds of the invention. Each of these dosage form technologies is described in detail in “Drugs and the Pharmaceutical Sciences”, Edited by James Swarbrick, Marcel Dekker, New York.

Materials:

Conventional enteric protectant polymers or mixtures of polymers for use herein include insoluble at a pH below about 5.5, i.e., that which is generally found in the stomach, but are soluble at pH about 5.5 or above, i.e., that present in the small intestine and the large intestine. The effectiveness of particular enteric protectant materials can be measured using known USP procedures.

Exemplary enteric protectant polymers employable in this embodiment include cellulose acetate phthalate, methyl acrylate-methacrylic acid copolymers, cellulose acetate succinate, hydroxypropylmethylcellulose phthalate, polyvinyl acetate phthalate, and methyl methacrylate-methacrylic acid copolymers. Another example is an anionic carboxylic copolymers based on methacrylic acid and methacrylate, commercially available as Eudragit(r). Typical examples include cellulose acetate phthalate (“CAP”), cellulose acetate trimellitate, hydroxypropyl methylcellulose phthalate (“HPMCP”), hydroxypropyl methylcellulose phthalate succinate, polyvinyl acetate phthalate (“PVAP”), methacrylic acid, and methacrylic acid esters. More typically the protectant is selected from, PVAP and/or HPMCP, particularly PVAP. PVAP is known under the trademark Sureteric(r), manufactured by Colorcon, Inc.

The enteric protectant materials may be applied to the vehicle with or without conventional plasticizers, such as acetylated mono glycerides, propylene glycol, glycerol, glyceryl triacetate, polyethylene glycol, triethyl citrate, tributyl citrate, diethyl phthalate, or dibutyl phthalate using methods known to those skilled in the art.

Exemplary Embodiments of Enteric Protection:

### Embodiment 1: Enteric Protected GS 4104 Capsules

In this exemplary embodiment, GS 4104 (compound 262, Example 116, phosphate salt form, 131.4 mg/capsule, 100 mg free base equivalent)) is mixed with Croscarmellose Sodium (2.6 mg/capsule) in a size 4 white opaque hard gelatin capsule shells (capsule composition: gelatin NF, titanium dioxide USP) and the capsule is enterically coated.

The following enteric coating formulations are applied to the capsule by procedures known to those in the art.

| Ingredients | % w/w |
|---|---|
| Preparation A: | |
| Hydroxypropyl methylcellulose | 5.0 |
| phthalate (“HPMCP”) | |
| Triacetin | 0.5 |
| Alcohol USP | 7.9 |
| Water | 15.5 |
| Preparation B: | |
| HPMCP | 10.0 |
| Titanium dioxide | 0.2 |
| Dimethyl polysiloxane | 0.05 |
| Triethyl citrate | 1.0 |
| Alcohol USP | 72.75 |
| Water | 16.00 |
| Preparation C: | |
| Cellulose acetate phthalate (“CAP”) | 8.5 |
| Diethyl phthalate | 1.5 |
| Titanium dioxide | 0.2 |
| Acetone | 44.9 |
| Denatured alcohol | 44.9 |
| Preparation D: | |
| Polyvinyl acetate phthalate (”PVAP”) | 5.0 |
| Acetylated glycerides | 0.8 |
| Methylene chloride | 47.1 |
| Denatured alcohol | 47.1 |
| Preparation E: | |
| Methacrylic acid or methacrylic | 8.0 |
| acid ester (Eudragit (r) S or L, manufactured by | |
| Rohm Pharma, GMBH, Wetterstadt, | |
| West Germany) | |
| Acetone | 46.0 |
| Anhydrous alcohol | 46.0 |
| Plasticizer | q.s. |

Typically the enteric polymer (with or without plasticizer) is dissolved in the solvents described under each formulation to form a suspension/solution. Optionally, an opacifer such as titanium dioxide is added. The vehicle is sprayed with the coating suspension/solution in a suitable vessel under conditions such that an enterically-protected coating is laid down on the vehicle without dissolving or disrupting the vehicle. Approximately 1-50%, typically 1-15%, more typically, 5-10% by weight of the finished coated vehicle of the enteric polymer coating will be useful for adequate enteric protection.

### Embodiment 2: Enteric Protected Tablet

In another exemplary embodiment a core tablet is encased within an enteric coating. Optionally, a subcoating is used.

Core Tablets:

Core tablets of the present invention may be formed by combining (a) the active ingredient with pharmaceutically-acceptable excipients in a mixture including for example: a diluent, a binder, a disintegrant, and optionally one or more ingredients selected from a group consisting of: compression aids, flavors, flavor enhancers, sweeteners, dyes, pigments, buffer systems, and preservatives; (b) lubricating the mixture with a lubricant; and (c) compressing the resultant lubricated mixture into a desired tablet form using various tableting techniques available to those skilled in the art. The term “tablet” as used herein is intended to encompass compressed or formed pharmaceutical dosage formulations of all shapes and sizes.

Typical diluents employable in this embodiment include lactose or microcrystalline cellulose.

Typical binders employable in this embodiment include, but are not limited to, povidone. Povidone is available under the trade name “Avicel” from ISP Corporation.

The disintegrant may be one of several modified starches, or modified cellulose polymers. Typically, croscarmellose sodium is used. Croscarmellose sodium NF Type A is commercially available under the trade name “Ac-di-sol”.

Typical lubricants include magnesium stearate, stearic acid, hydrogenated vegetable oil or talc.

Flavoring agents include those described in Remington's Pharmaceutical Sciences, 18th Edition, Mack Publishing Company, 1990, pp. 1288-1300.

Typical sweeteners include saccharin, Aspartame, or edible mono- or disaccharides such as glucose or sucrose.

Dyes and pigments include those described in the Handbook of Pharmaceutical Excipients, pp. 81-90, 1986 by the American Pharmaceutical Association & the Pharmaceutical Society of Great Britain.

Typical preservatives include methyl paraben, propyl paraben, cetylpyridinium chloride, and the salts thereof, sorbic acid and the salts thereof, thimerosal, or benzalkonium chloride.

Enteric Coating:

Eudragit L-30-D(r), a methacrylic acid copolymer, manufactured by Rohm Pharma GmbH, Weiterstadt, West Germany, is a suitable enteric polymer. Eudragit L-30-D(r) has a ratio of free carboxyl groups to ester groups of approximately 1:1 and is freely soluble at pH 5.5 and above. In general, the greater the percentage of Eudragit L-30-D(r) contained in the enteric coating, the more proximal the release of active in the lower gastrointestinal tract. The location in the lower gastrointestinal tract at which the coating releases the compound can be manipulated by one skilled in the art through control of the composition and thickness of the applied enteric coating.

Typically a plasticizer, such as those set forth above, is included.

Other additives such as talc or silica may be used as detackifiers to improve the coating process.

Sub coating:

Optionally a stability enhancing subcoat on the core tablet is used to minimize interaction between the compound of this invention and the enteric coating. This also permits utilization of a single 10-300 micron thick enteric film without affecting product stability. This subcoat inhibits migration of active ingredient from the core tablet into the enteric coating, thus improving shelf life and product stability, but the subcoat rapidly dissolves in intestinal fluid once the exterior enteric coating has been breached.

Typical subcoating polymers employable in this embodiment include hydroxypropyl methylcellulose, hydroxypropyl cellulose, hydroxypropyl ethylcellulose, or polyvinylpyrrolidone.

### EXAMPLES

### General

The following Examples refer to the Schemes.

Some Examples have been performed multiple times. In repeated Examples, reaction conditions such as time, temperature, concentration and the like, and yields were within normal experimental ranges. In repeated Examples where significant modifications were made, these have been noted where the results varied significantly from those described. In Examples where different starting materials were used, these are noted. When the repeated Examples refer to a “corresponding” analog of a compound, such as a “corresponding ethyl ester”, this intends that an otherwise present group, in this case typically a methyl ester, is taken to be the same group modified as indicated. For example, the “corresponding ethyl ester of compound 1” is

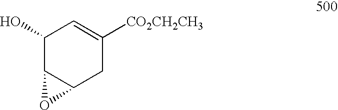

### Example 1

Epoxy alcohol 1: Prepared from shikimic acid by the procedure of McGowan and Berchtold, “J. Org. Chem.”, 46:2381 (1981).

### Example 2

Epoxy allyl ether 2: To a solution of epoxy alcohol 1 (2.37 g, 14.08 mmol) in dry benzene (50 mL) was added thallium(I)ethoxide (1.01 mL) in one portion. After 2 hr the reaction was concentrated in vacuo and the residue dissolved in acetonitrile. Allyl iodide (3.0 mL) was added and the mixture was stirred in the dark for 16 h. The solids were filtered thru a celite pad and washed with chloroform. Concentration in vacuo followed by flash chromatography (40% EtOAc in hexane) gave 1.24 g (42%) of 2 as a pale viscous oil. ¹H NMR (300 MHz, CDCl₃): δ 6.75 (1H, m); 6.10-5.90 (1H, m, —CH═, allyl); 5.40-5.15 (2H, m, ═CH₂, allyl); 4.47-4.43 (1H, m); 4.30-4.15 (2H, m, —CH₂—, allyl); 3.73 (3H, s); 3.55-3.50 (1H, m); 3.45-3.40 (1H, m); 3.15-3.00 (1H, dm, J=19.5 Hz), 2.50-2.35 (1H, dm, J=2.7, 19.5 Hz).

### Example 3

Azido alcohol 3: Epoxide 2 (1.17 g, 5.57 mmol), sodium azide (1.82 g) and ammonium chloride (658 mg) were refluxed in MeOH/H₂O (8:1) (35 mL) for 18 h. The reaction was then concentrated in vacuo and the residue partitioned between ethyl ether and water. The organic layer was washed with brine and dried. Concentration in vacuo gave 3 as a pale oil 1.3 g (92%) which was used without further purification. ¹H NMR (300 MHz, CDCl₃): δ 6.95-6.85 (1H, m); 6.00-5.85 (1H, m, —CH═, allyl); 5.35-5.25 (2H, m, ═CH₂, allyl); 4.25-4.10 (2H, m, —CH₂—, allyl); 4.12 (1H, bt, J=4.2 Hz); 3.95-3.75 (2H, m); 3.77 (3H, s); 2.85 (1H, dd, J=5.3, 18.3 Hz); 2.71 (1H, bs); 2.26 (1H, dd, J=7.2, 18.3 Hz).

### Example 4

Aziridine 4: To a solution of alcohol 3 (637 mg, 2.52 mmol) in CH₂Cl₂ (20 mL) cooled to 0° C. was added DMAP (few crystals) and triethyl amine (442 μL). MsCl (287 μL) was then added and the reaction stirred for 2 h at 0° C. Volatiles were removed and the residue partitioned between ethyl ether and water. The organic layer was washed with saturated bicarbonate, brine and then dried. Concentration in vacuo gave 881 mg of crude mesylate. ¹H NMR (300 MHz, CDCl₃): δ 6.87-6.84 (1H, s); 6.00-5.85 (1H, m, —CH═, allyl); 5.40-5.25 (2H, m, ═CH₂, allyl); 4.72 (1H, dd, J=3.9, 8.5 Hz); 4.32 (1H, bt, J=3.9 Hz); 4.30-4.15 (2H, m, —CH₂—, allyl); 3.77 (3H, s); 3.14 (3H, s); 2.95 (1H, dd, J=5.7, 18.6 Hz); 2.38 (1H, dd, J=6.7, 18.6 Hz).

The crude mesylate was dissolved in dry THF (20 mL) and treated with Ph₃P (727 mg). After stirring for 3 h at room temperature, water (15 mL) and solid NaHCO₃ (1.35 g) was added and the mixture stirred overnight at room temperature. The reaction was then concentrated in vacuo and the residue partitioned between EtOAc, saturated bicarbonate and brine. The organic layer was separated and dried over MgSO₄. Concentration in vacuo and flash chromatography of the residue gave the aziridine 4 170 mg (33%) as a pale yellow oil. ¹H NMR (300 MHz, CDCl₃): δ 6.82-6.80 (1H, m); 6.04-5.85 (1H, m, —CH═, allyl); 5.35-5.20 (2H, m, ═CH₂, allyl); 4.39 (1H, bd, J=2.4 Hz); 4.20-4.05 (2H, m, —CH₂-allyl); 3.73 (3H, s); 2.90-2.80 (1H, bd, J=18.9 Hz); 2.65-2.40 (2H, m).

### Example 5

N-acetyl aziridine 5: Aziridine 4 (170 mg, 0.814 mmol) was dissolved in CH₂Cl₂ (2 mL) and pyridine (4 mL) and cooled to 0° C. Acetyl chloride (87 μL) was then added and the reaction stirred at 0° C. for 1 h. Volatiles were removed in vacuo and the residue partitioned between ethyl ether, saturated bicarbonate and brine. The organic layer was separated and dried over MgSO₄. Concentration gave crude 5 196 mg (96%) which was used without further purification. ¹H NMR (300 MHz, CDCl₃): δ 6.88-6.86 (1H, m); 6.00-5.85 (1H, m, —CH═, allyl); 5.40-5.20 (2H, m, ═CH₂, allyl); 4.45-4.40 (1H, m); 4.16 (2H, d, J=6.0 Hz, —CH₂—, allyl); 3.76 (3H, s); 3.00-2.95 (2H, m); 2.65 (1H, bd, J=18.5 Hz); 2.14 (3H, s).

### Example 6

Azido allyl ether 6: Aziridine 5 (219 mg, 0.873 mmol), sodium azide (426 mg) and ammonium chloride (444 mg) in dry DMF (7 mL) was heated at 65° C. under argon overnight. The reaction was poured into saturated bicarbonate/brine and extracted with ethyl ether several times. The combined ether layers were washed with brine and dried. Concentration followed by flash chromatography (EtOAc only) gave the azido amine 77 mg (35%) which was dissolved in CH₂Cl₂ (1 mL) and pyridine (1 mL) and cooled to 0° C. Acetyl chloride (38 μL) was added and after 45 min solid NaHCO₃ was added and the volatiles removed under vacuum. The residue was partitioned between EtOAc and brine. The organic layer was dried over MgSO₄ and concentrated in vacuo. Flash chromatography (EtOAc only) gave 6 90 mg (99%). ¹H NMR (500 MHz, CDCl₃): δ 6.86 (1H, bt, J=2.2 Hz); 5.95-5.82 (1H, m, CH═, allyl); 5.68 (1H, bd, J=7.3 Hz); 5.35-5.20 (2H, m, ═CH₂, allyl); 4.58-4.52 (1H, m); 4.22-4.10 (2H, m); 4.04 (1H, dd, J=5.9, 12.5 Hz); 3.77 (3H, s); 3.54-3.52 (1H, m); 2.89 (1H, dd, J=5.9, 17.6 Hz); 2.32-2.22 (1H, m); 2.06 (3H, s).

### Example 7

Azido diol 7: To a solution of olefin 6 (90 mg, 0.306 mmol) in acetone (3 mL) and water (258 μL) was added N-methyl morpholine-N-oxide (39 mg) and OsO₄ (73 μL of a 2.5 % w/w in t-butanol). The reaction was then stirred at room temperature for 3 days. Solid sodium hydrosulfite was added and after stirring for 20 min the reaction was filtered thru a celite pad and washed with copious amounts of acetone. Concentration in vacuo followed by flash chromatography (10% MeOH in CH₂Cl₂) gave the diol 7 50 mg (50%). ¹H NMR (300 MHz, CD₃CN): δ 6.80-6.70 (1H, m); 4.20-4.15 (1H, bm); 3.95-3.80 (1H, m); 3.80-3.25 (6H, m); 3.70 (3H, s); 3.10 (1H, bs); 2.85 (1H, bs); 2.85-2.75 (1H, m); 2.30-2.15 (1H, m); 2.16 (1H, bs); 1.92 (3H, s).

### Example 8

Amino acid diol 8: A solution of the diol 7 (23 mg, 0.07 mmol) in THF (1 mL) was treated with aq. KOH (223 μL, of 0.40 M solution) at room temperature. After stirring for 1.5 h the reaction was acidified to pH=4 with Amberlite IR-120 (plus) ion exchange resin. The resin was filtered and washed with MeOH. Concentration in vacuo gave the crude carboxylic acid which was dissolved in ethanol (1.5 mL). To this solution was added Lindlar's catalyst (20 mg) and the reaction stirred over a hydrogen atmosphere (1 atm via a balloon) for 20 h. The reaction mixture was filtered thru a celite pad and washed with hot ethanol and water. The ethanol was removed under vacuum and the resulting aqueous layer lyophilized to give a mixture of the desired amino acid 8 and the starting azide 7 as a white powder. Compound 8: ¹H NMR (500 MHz, D₂O): δ 6.5 (1H, s); 4.24-4.30 (2H, m); 4.25-4.18 (1H, m); 3.90-3.55 (5H, complex m); 2.96-2.90 (1H, m); 2.58-2.50 (1H, complex m); 2.12 (3H, s).

### Example 9

Compound 62: A suspension of Quinic acid (60 g), cyclohexanone (160 mL) and toluenesulfonic acid (600 mg) in benzene (450 mL) was refluxed with Dean-Stark for 14 hrs. The reaction mixture was cooled to room temperature and poured into saturated NaHCO₃ solution (150 mL). The aqueous layer was extracted with CH₂Cl₂ (3×). The combined organic layers were washed with water (2×), brine (1×), and dried over Na₂SO₄. Concentration gave a whited solid, which was recrystallized from ether (75 g, 95%): ¹H NMR (CDCl₃) δ 4.73 (dd, J=6.1, 2.5 Hz, 1H), 4.47 (ddd, J=7.0, 7.0, 3.0 Hz, 1H), 4.30 (ddd, J=5.4, 2.6, 1.4 Hz, 1H), 2.96 (s, 1H), 2.66 (d, J=11.7 Hz, 1H), 2.40-2.15 (m, 3H), 1.72-1.40 (m, 10H).

### Example 10

Compound 63: To a solution of lactone 62 (12.7 g, 50 mmol) in methanol (300 mL) was added sodium methoxide (2.7 g, 50 mmol) in one portion. The mixture was stirred at room temperature for 3 hrs, and quenched with acetic acid (3 mL) and stirred for 10 min. The mixture was poured into saturated NH₄Cl solution (300 mL), and extracted with CH₂Cl₂ (3×). The combined organic phase was washed with brine (1×), and dried over MgSO₄. Purification by flash column chromatography (Hexane/EtOAc=1/1 to 1/2) gave diol (11.5 g, 80%) and starting material (1.2 g, 10%): ¹H NMR (CDCl₃) δ 4.47 (ddd, J=7.4, 5.8, 3.5 Hz, 1H), 4.11 (m, 1H), 3.98 (m, 1H), 3.81 (s, 3H), 3.45 (s, 1H), 2.47 (d, J=3.3 Hz, 1H), 2.27 (m, 2H), 2.10 (dd, J=11.8,4.3 Hz, 1H), 1.92-1.26 (m, 10H).

### Example 11

Compound 64: To a mixture of diol 63 (1.100 g, 3.9 mmol), molecule sieves (3 A, 2.2 g) and pyridine (1.1 g) in CH₂Cl₂ (15 mL) was added PCC (3.3 g, 15.6 mmol) in one portion. The mixture was stirred at room temperature for 26 hrs, and diluted with ether (30 mL). The suspension was filtered through a pad of celite, and washed with ether (2×20 mL). The combined ether was washed with brine (2×), and dried over MgSO₄. Concentration and purification was by flash column chromatography (Hexane/EtOAc=3/1) gave the ketone (0.690 g, 67%): ¹H NMR (CDCl₃) δ 6.84 (d, J=2.8 Hz, 1H), 4.69 (ddd, J=6.4, 4.9, 1.6 Hz, 1H), 4.30 (d, J=5.0 Hz, 1H), 3.86 (s, 3H), 3.45 (d, J=22.3 Hz, 1H), 2.86 (m, 1H), 1.69-1.34 (m, 10H).

### Example 12

Compound 28: To a solution of ketone 64 (0.630 g, 2.4 mmol) in MeOH (12 mL) at 0° C. was added NaBH₄ in 30 min. The mixture was stirred for additional 1.5 hrs at 0° C., and quenched with 15 mL of saturated NH₄Cl solution. The solution was extracted with CH₂Cl₂ (3×), and the combined organic extract was dried over MgSO₄. Purification by flash column chromatography (Hexane/EtOAc=2/1) gave the alcohol (0.614 g, 97%): ¹H NMR (CDCl₃) δ 6.94 (d, J=0.5 Hz, 1H), 4.64 (ddd, J=9.8, 6.7, 3.2 Hz, 1H), 4.55 (dd, J=7.1, 4.2 Hz, 1H), 4.06 (m, 1H), 3.77 (s, 3H), 3.04 (dd, J=16.5, 2.1 Hz, 1H), 2.73 (d, J=10.2 Hz, 1H), 1.94 (m, 1H), 1.65-1.29 (m, 10H).

### Example 13

Compound 66: Alcohol 28 (2.93 g, 10.9 mmol) and toluenesulfonic acid (1.5 g) were dissolved in acetone (75 mL), and the mixture was stirred at room temperature for 15 hrs. The reaction was quenched with water (30 mL), and basified with concentrated NH₃—H₂O until PH=9. Acetone was removed under reduced pressure, and the water phase was extracted with CH₂Cl₂ (3×). The combined organic extracts were washed with brine (1×), and dried over Na₂SO₄. Concentration gave the desired product: ¹H NMR (CDCl₃) δ 7.01 (m, 1H), 4.73 (m, 1H), 4.42 (m, 1H), 3.97 (m, 1H), 3.76 (s, 3H), 2.71-2.27 (m, 2H), 2.02 (s, 3H), 1.98 (s, 3H).

### Example 14

Compound 67: To a solution of alcohol 66 (10.9 mmol) in CH₂Cl₂ (60 mL) at 0° C. was added pyridine (4.4 mL, 54.5 mmol), followed by addition of trimethylacetyl chloride (2.7 mL, 21.8 mmol). The mixture was warmed to room temperature and stirred for 14 hrs. The mixture was diluted with CH₂Cl₂, and washed with water (2×), brine (1×), and dried over MgSO₄. Purification by flash column chromatography (Hexane/EtOAc=9/1) gave the diester (2.320 g, 68%): ¹H NMR (CDCl₃) δ 6.72 (m, 1H), 5.04 (m, 1H), 4.76 (m, 1H), 4.40 (m, 1H), 3.77 (s, 3H), 2.72-2.49 (m, 2H), 1.37 (s, 3H), 1.35 (s, 3H), 1.23 (s, 9H).

### Example 15

Compound 68: Diester 67 (2.32 g, 2.3 mmol) was dissolved in acetone/H₂O (1/1, 100 mL) and heated at 55° C. for 16 hrs. Solvents were removed, water (2×50 mL) was added and evaporated. Concentration with toluene (2×50 mL) gave diol, which was used without further purification: ¹H NMR (CDCl₃) δ 6.83 (m, 1H), 5.06 (m, 1H), 4.42 (m, 1H), 4.09 (m, 1H), 3.77 (s, 3H), 2.68-2.41 (m, 2H), 1.22 (s, 9H).

### Example 16

Compound 69: To a solution of diol 68 (0.410 g, 1.5 mmol) in THF (8 mL) at 0° C. was added triethylamine (0.83 mL, 6.0 mmol), followed by slow addition of thionyl chloride (0.33 mL, 4.5 mmol). The mixture was warmed to room temperature and stirred for 3 hrs. The mixture was diluted with CHCl₃, and washed with water (3×), brine (1×), and dried over MgSO₄. Purification by flash column chromatography (Hexanes/EtOAc=5/1) gave a exo/endo mixture (0.430 g, 90%): ¹H NMR (CDCl₃) δ 6.89-6.85 (m, 1H), 5.48-4.84 (m, 3H), 3.80, 3.78 (s, 3H), 2.90-2.60 (m, 2H), 1.25, 1.19 (s, 9H).

### Example 17

Compound 70: The mixture of sulfone 69 (0.400 g, 1.3 mmol) and sodium azide (0.410 g, 6.29 mmol) in DMF (10 mL) was stirred for 20 hrs. The reaction mixture was then diluted with ethyl acetate, washed with saturated NH₄Cl solution, water, brine, and dried over MgSO₄. Concentration gave the azide (0.338 g, 90%): ¹H NMR (CDCl₃) δ 6.78 (m, 1H), 5.32 (m, 1H), 4.20 (m, 1H), 3.89 (m, 1H), 3.78 (s, 3H), 3.00-2.60 (m, 2H), 1.21 (s, 9H).

### Example 18

Compound 71: To a solution of alcohol 70 (0.338 g, 1.1 mmol) in CH₂Cl₂ (11 mL) at 0° C. was added triethylamine (0.4 mL, 2.9 mmol), followed by slow addition of methylsulfonic chloride (0.18 mL, 2.3 mmol). The mixture was stirred at 0° C. for 30 min., and diluted with CH₂Cl₂. The organic layer was washed with water (2×), brine, and dried over MgSO₄. Purification by flash column chromatography (Hexane/EtOAc=3/1) gave the desired compound (0.380 g, 82%): ¹H NMR (CDCl₃) δ 6.82 (m, 1H), 5.44 (m, 1H), 4.76 (dd, J=7.3, 1.4 Hz, 1H), 4.48 (m, 1H), 3.80 (s, 3H), 3.11 (s, 3H), 2.82-2.61 (m, 2H), 1.21 (s, 9H).

### Example 19

Compound 72: The mixture of azide 71 (0.380 g, 0.94 mmol) and triphenylphosphine (0.271 g, 1.04 mmol) in THF (19 mL) was stirred for 2 hrs. The reaction was quenched with water (1.9 mL) and triethylamine (0.39 mL, 2.82 mmol), and the mixture was stirred for 14 hrs. Solvents were removed under reduced pressure, and the mixture was used for next step. To a solution of above mixture in CH₂Cl₂ (20 mL) at 0° C. was added pyridine (0.68 mL, 8.4 mmol), followed by slow addition of acetyl chloride (0.30 mL, 4.2 mmol). The mixture was stirred at 0° C. for 5 min., and diluted with ethyl acetate. The mixture was washed with water (2×), brine (1×), dried over MgSO₄. Purification by flash column chromatography (Hexanes/EtOAc=3/1) gave the aziridine (0.205 g, 83%): ¹H NMR (CDCl₃) δ 7.19 (m, 1H), 5.58 (m, 1H), 3.77 (s, 3H), 3.14 (m, 2H), 2.85 (dd, J=7.0, 1.6 Hz, 1H), 2.34 (m, 1H), 2.16 (s, 3H), 1.14 (s, 9H).

### Example 20

Compound 73: The mixture of aziridine 72 (0.200 g, 0.68 mmol), sodium azide (0.221 g, 3.4 mmol), and ammonium chloride (0.146 g, 2.7 mmol) in DMF (10 mL) was stirred at room temperature for 14 hrs. Then the mixture was diluted with ethyl acetate, and washed with water (5×), brine (1×), and dried over MgSO₄. Purification by flash column chromatography (hexanes/EtOAc=2/1) gave desired product and deacetyl amine (0.139 g). The mixture was dissolved in acetic anhydride (2 mL), and stirred for 2 hrs. Excess anhydride was removed under reduced pressure, and give the desired product (149 mg): ¹H NMR (CDCl₃) δ 6.76 (m, 1H), 5.53 (d, J=8.5 Hz, 1H), 5.05 (m, 1H), 4.31 (m, 1H), 4.08 (m, 1H), 3.79 (s, 3H), 2.91 (m, 1H), 2.51 (m, 1H), 1.99 (s, 3H), 1.20 (s, 9H).

### Example 21

Compound 74: A solution of potassium hydroxide in MeOH/H₂O (0.5 M, 4.4 mL, 2.2 mmol) was added to ester 73 (149 mg, 0.44 mmol) and the mixture was stirred at room temperature for 3 hrs. The mixture was cooled to 0° C., and acidified with Amberlite (acidic) to PH=3-4. The mixture was filtered, and washed with MeOH. Concentration gave the carboxylic acid as a white solid (73 mg, 69%): ¹H NMR (CD₃OD) δ 6.62 (m, 1H), 4.15 (m, 1H), 3.95-3.72 (m, 2H), 2.84 (dd, J=6.7, 1.4 Hz, 1H), 2.23 (m, 1H), 1.99 (s, 3H).

### Example 22

Compound 75: The mixture of azide 74 (8 mg) and Pd—C (Lindlar) (15 mg) in ethanol (2 mL) was stirred under hydrogen for 16 hrs. The mixture was filtered through celite, washed with hot MeOH—H₂O (1/1). Concentration gave a solid. The solid was dissolved in water, and passed through a short C-8 column, and washed with water. Concentration gave a white solid (6 mg): ¹H NMR (D₂O) δ 6.28 (m, 1H), 4.06-3.85 (m, 3H), 2.83 (dd, J=17.7, 5.4 Hz, 1H), 2.35 (m, 1H), 2.06 (s, 3H).

### Example 23

Compound 76: Carboxylic acid 74 (68 mg, 0.28 mmol) and diphenyldiazomethane (61 mg, 0.31 mmol) were dissolved in ethanol (12 mL), and stirred for 16 hrs. The reaction was quenched with acetic acid (0.5 mL), and the mixture was stirred for 10 min. Solvents were removed under reduced pressure. Purification by flash column chromatography (EtOAc) gave the ester (56 mg, 50%): ¹H NMR (CD₃OD) δ 7.36-7.23 (m, 10H), 6.88 (s, 1H), 6.76 (s, 1H), 4.21 (m, 1H), 3.93-3.79 (m, 2H), 2.89 (dd, J=17.7, 5.0 Hz, 1H), 2.34 (m, 1H), 2.00 (s, 3H).

### Example 24

Compound 77: To a solution of alcohol 76 (20 mg, 0.05 mmol) in CH₂Cl₂ (1 mL) was added pyridine (40 μL, 0.5 mmol), followed by addition of acetic anhydride (24 μL, 0.25 mmol). The mixture was stirred for 24 hrs, and solvents and reagents were removed under reduced pressure. Purification by flash column chromatography (Hexane/EtOAc=1/2) gave the diester (20 mg, 91%): ¹H NMR (CDCl₃) δ 7.40-7.27 (m, 10H), 6.95 (s, 1H), 6.87 (m, 1H), 5.60 (m, 1H), 5.12 (ddd, J=16.4, 10.2, 5.9 Hz, 1H), 4.28 (dd, J=20.0, 9.4 Hz, 1H), 4.15 (m, 1H), 2.93 (dd, J=17.8, 5.2 Hz, 1H), 2.57 (m, 1H), 2.09 (s, 3H), 2.01 (s, 3H).

### Example 25

Compound 78: The mixture of diester 77 (20 mg, 0.045 mmol), anisole (50 μL, 0.45 mmol), and TFA (1 mL) in CH₂Cl₂ (1 mL) was stirred for 20 min. Solvents and reagents were removed under reduced pressure. Purification by flash column chromatography (EtOAc to EtOAc/AcOH=100/1) gave the carboxylic acid (6 mg): ¹H NMR (CDCl₃) δ 6.85 (m, 1H), 5.54 (m, 1H), 5.12 (m, 1H), 4.31-4.03 (m, 2H), 2.89 (m, 1H), 2.60-2.41 (m, 1H), 2.11 (s, 3H), 2.03 (s, 3H).

### Example 26

Compound 79: The mixture of azide 78 (6 mg, 0.02 mmol) and Pd—C (Lindlar) (15 mg) in EtOH/H₂O (2.2 mL, 10/1) was stirred under hydrogen for 3 hrs. The mixture was filtered through a pad of celite, washed with hot MeOH/H₂O (1/1). Evaporation gave a white solid. The solid was dissolved in water, and passed through a C-8 column. Evaporation of water gave a white powder (3 mg): ¹H NMR (D₂O) δ 6.32 (m, 1H), 5.06 (m, 1H), 4.06 (t, J=10.4 Hz, 1H), 3.84 (m, 1H), 2.83 (m, 1H), 2.42 (m, 1H), 2.06 (s, 3H), 2.00 (s, 3H).

### Example 27

Compound 80: To a solution of alcohol 76 (35 mg, 0.086 mmol), Boc-glycine (30 mg, 0.172 mmol), and catalytic amount DMAP in CH₂Cl₂ (1 mL) was added DCC (35 mg, 0.172 mmol). The mixture was stirred for 30 min, and filtered and washed with CHCl₃. The CHCl₃ solution was washed with water (2×). Concentration gave a white solid. Purification by flash column chromatography (Hexane/EtOAc=1/2) gave product (30 mg): ¹H NMR (CDCl₃) δ 7.39-7.26 (m, 10H), 6.95 (s, 1H), 6.86 (m, 1H), 5.77 (m, 1H), 5.27 (m, 1H), 4.99 (m, 1H), 4.18-4.01 (m, 2H), 3.94-3.84 (m, 2H), 2.96 (dd, J=7.8, 5.9 Hz, 1H), 2.57 (m, 1H), 2.02 (s, 3H), 1.45 (s, 9H).

### Example 28

Compound 81: The mixture of diester 80 (30 mg, 0.05 mmol), anisole (150 μL), and TFA (1 mL) in CH₂Cl₂ (1 mL) was stirred for 3 hrs. Solvents and reagents were evaporated. The mixture was dissolved in water, and washed with CHCl₃ (3×). Water phase was evaporated to gave a white solid (15 mg): ¹H NMR (CD₃OD) δ 6.73 (m, 1H), 5.25-5.15 (m, 1H), 4.35 (m, 1H), 4.17 (m, 1H), 3.82 (m, 2H), 2.93 (dd, J=17.7, 5.6 Hz, 1H), 2.42 (m, 1H), 1.97 (s, 3H).

### Example 29

Compound 82: The mixture of azide 81 (15 mg, 0.05 mmol) and Pd—C (Lindlar) (30 mg) in EtOH/H₂O (4 mL, 1/1) was stirred under hydrogen for 3 hrs. The mixture was filtered through a pad of celite, and washed with hot MeOH/H₂O (1/1). Concentration gave a glass-like solid. The solid was dissolved in water, and passed through C-8 column. Evaporation of water gave the amino acid: ¹H NMR (D₂O) δ 6.68 (m, 1H), 5.28 (m, 1H), 4.29 (m, 1H), 4.08-3.79 (m, 3H), 2.85 (m, 1H), 2.41 (m, 1H), 2.04 (s, 3H).

### Example 30

bis-Boc guanidinyl methyl ester 92: Treated according to the procedure of Kim and Qian, “Tetrahedron Lett.”, 34:7677 (1993). To a solution of amine 91 (42 mg, 0.154 mmol), bis-Boc thiourea (43 mg, 0.155 mmol) and triethylamine (72 μL) in dry DMF (310 μL) cooled to 0° C. was added mercury chloride (46 mg, 0.170 mmol) in one portion. After 30 min the reaction was warmed to room temperature and stirred for an additional 2.5 h. The reaction mixture was then filtered through a celite pad, concentrated and purified by flash column chromatography (100% ethyl acetate) to give 70 mg (89%) of 92 as a colorless foam. ¹H NMR (CDCl₃, 300 MHz): δ 11.37 (s, 1H); 8.60 (d, 1H, J=7.8 Hz); 6.83 (t, 1H, J=2.1 Hz); 6.63 (d, 1H, J=8.4 Hz); 4.76 (d, 1H, J=7.0 Hz); 4.71 (d, 1H, J=7.0 Hz); 4.45-4.10 (complex m, 2H); 3.76 (s, 3H); 3.39 (s, 3H); 2.84 (dd, 1H, J=5.4, 17.4 Hz); 2.45-2.30 (m, 1H); 1.92 (s, 3H); 1.49 (s, 18H).

### Example 31

bis-Boc guanidinyl carboxylic acid 93: To a solution of ester 92 (70 mg, 0.136 mmol) in THF (3 mL) cooled to 0° C. was added aq. KOH (350 μL of a 0.476 M solution). The reaction was then warmed to room temperature and stirred for 2 h. The reaction was then acidified to pH=4.5 with Amberlite IR-120 (plus) acidic resin. The resin was then filtered and washed with ethanol and H₂O. Concentration in vacuo gave 66 mg (97%) of carboxylic acid 93 as a white solid. ¹H NMR (CDCl₃, 300 MHz): δ 11.40 (br s, 1H); 8.67 (d, 1H, J=7.8 Hz); 6.89 (s, 1H); 6.69 (br d, 1H, J=8.4 Hz); 4.77 (d, 1H, J=7.2 Hz); 4.70 (d, 1H, J=7.2 Hz); 4.40-4.15 (m, 2H); 3.39 (s, 3H); 2.84 (dd, 1H, J=4.8, 17.1 Hz); 2.45-2.30 (m, 1H); 1.95 (s, 3H); 1.49 (s, 9H); 1.48 (s, 9H).

### Example 32

Guanidine carboxylic acid TFA salt 94: To a solution of bis-Boc guanidinyl carboxylic acid 93 (23 mg, 0.046 mmol) in CH₂Cl₂ (1 mL) cooled to 0° C. was added neat trifluoroacetic acid (500 μL). After 30 min the reaction was warmed to room temperature and stirred for an additional 1.25 h. Volatiles were removed under vacuum and the residue co-evaporated with several portions of H₂O to give a pale orange solid. The residue was purified by reverse phase C₁₈ chromatography using H₂O as an eluent. Fractions containing the desired product were pooled and lyophilized to give 15 mg of 93 as a white powder. ¹H NMR (D₂O, 500 MHz): δ 6.82 (t, 1H, J=2.0 Hz); 4.51-4.47 (m, 1H); 3.93 (dd, 1H, J=9.0, 11.2 Hz); 3.87-3.80 (apparent ddd, 1H); 2.88 (m, 1H); 2.48-2.45 (complex m); 2.07 (s, 3H). ¹³C NMR (D₂O): δ 176.1; 170.0; 157.1; 139.2; 129.5; 69.4; 56.2; 50.9; 30.3; 22.2.

### Example 33

Synthesis of 102: A solution of azido allyl ether 6 (24 mg, 0.082 mmol) in ethanol (1 mL) was treated with hydrogen gas (1 atm) over Lindlar's catalyst (30 mg) for 1.5 h. The reaction mixture was filtered through a celite pad and washed with hot ethanol. Concentration in vacuo gave a pale solid which was dissolved in THF (1.5 mL) and treated with aqueous KOH (246 μL of a 0.50 M solution). After stirring at ambient temperature for 2 h the reaction was acidified to pH=4.0 with Amberlite IR-120 (plus) acidic resin, filtered and washed with ethanol and H₂O. Concentration in vacuo gave an orange solid which was purified by a C₁₈ column chromatography eluting with H₂O. Fractions containing the product were pooled and lyophilized to give a 2 to 1 mixture of 102 and the fully saturated compound 103 as a white powder. ¹H NMR data for compound 102: ¹H NMR (D₂O, 500 MHz): δ: 7.85 (s, 1H); 4.29 (br d, 1H, J=9.2 Hz); 4.16 (dd, 1H, J=11.6, 11.6 Hz); 3.78-3.72 (m, 2H); 3.62 (apparent ddd, 1H); 2.95 (apparent dd, 1H); 2.58-2.52 (m, 1H); 2.11 (s, 3H); 1.58 (q, 2H, J=7.3 Hz); 0.91 (t, 3H, J=7.3 Hz).

### Example 34

Synthesis of 115: A solution of amino acid 114 (10.7 mg, 0.038 mmol) in water (1.3 mL) cooled to 0° C. was adjusted to pH=9.0 with 1.0 M NaOH. Benzyl formimidate hydrochloride (26 mg, 0.153 mmol) was then added in one portion and the reaction stirred between 0-5° C. for 3 h while maintaining the pH between 8.5-9.0 with 1.0 M NaOH. The reaction was then concentrated in vacuo and the residue applied to a C₁₈ column and eluted with water. Fractions containing the product were pooled and lyophilized to give the formamidine carboxylic acid 115 (10 mg) as a white powder. ¹H NMR (D₂O, 300 MHz, mixture isomers): δ 7.83 (s, 1H); [6.46(s) & 6.43 (s); 1H total]; 4.83 (d, 1H, J=7.3 Hz); 4.73 (d, 1H, J=7.3 Hz); 4.50-4.35 (m, 1H); 4.10-4.05 (m, 1H); [4.03-3.95 (m) & 3.80-3.65 (m), 1H total]; 3.39 (s, 3H); 2.90-2.75 (m, 1H); 2.55-2.30 (m, 1H); [2.03 (s) & 2.01 (s), 3H total].

### Example 35

Compound 123: To a solution of alcohol 63 (5.842 g, 20.5 mmol) and DMAP (200 mg) in pyridine (40 mL) was added tosyl chloride (4.3 g, 22.6 mmol). The mixture was stirred at room temperature for 40 hrs, and pyridine was removed under reduced pressure. The reaction was quenched with water, and extracted with EtOAc (3×). The combined organic extracts were washed with water, brine, and dried over MgSO₄. Purification by flash column chromatography (Hexanes/EtOAc=2/1) gave the tosylate (8.04 g, 89%): ¹H NMR (CDCl₃) δ 7.84 (d, J=8.3 Hz, 2H), 7.33 (d, J=8.1 Hz, 2H), 4.78 (m, 1H), 4.43 (m, 1H), 4.06 (m, 1H), 3.79 (s, 3H), 2.44 (s, 3H), 2.43-1.92 (m, 4H), 1.61-1.22 (m, 10H).

### Example 36

Compound 124: To a solution of alcohol 123 (440 mg, 1.0 mmol) in pyridine (3 mL) was added POCl₃ (100 μL, 1.1 mmol). The mixture was stirred at room temperature for 12 hrs, and quenched with saturated NH₄Cl solution. The water phase was extracted with ether (3×). The combined ether layers were washed with water (2×), 2 N HCl solution (2×), brine, and dried over MgSO₄. Purification by flash column chromatography (Hexane/EtOAc=2/1) gave a mixture of the desired product 124 and some inpurity (350 mg, 83%, 2/1).

### Example 37

Compound 1: To a solution of the known acetonide of methyl shikimate (877 mg, 3.85 mmol, “Tetrahedron Lett.”, 26:21 (1985)) in dichloromethane (15 mL) at −10° C. was added methanesulfonyl chloride (330 μL, 4.23 mmol) followed by the dropwise addition of triethylamine (640 μL, 4.62 mmol). The solution was stirred at −10° C. for 1 h then at 0° C. for 2 h, at which time methanesulfonyl chloride (30 μL), triethylamine (64 μL) was added. After 1 h cold water was added, the organic phase was separated, washed with water, dried (MgSO₄), and evaporated. The crude product was chromatographed on silica gel (1/1-hexane/ethyl acetate) to provide mesylate 130 (1.1 g, 93%) as an oil. Mesylate 130 (990 mg, 3.2 mmol) was dissolved in tetrahydrofuran (5 mL) and was treated with 1M HCl (5 mL). The solution was stirred at room temperature for 19 h, diluted with water (5 mL) and stirred an additional 7 h. Evaporation of the organic solvent precipitated an oily residue which was extracted into ethyl acetate. The combined organic extracts were washed with brine, dried (MgSO₄), and evaporated. Addition of CH₂Cl₂ to the crude residue precipitated a white solid which was filtered and washed with CH₂Cl₂ to afford diol 131 (323 mg, 38%). To a partial suspension of diol 131 (260 mg, 0.98 mmol) in THF (5 mL) at 0° C. was added DBU (154 μL, 1.03 mmol). The solution was stirred at 0° C. for 3 h and then was warmed to room temperature stirring for 5 h. The solvent was evaporated and the crude residue was partitioned between ethyl acetate (40 mL) and 5% citric acid (20 mL). The organic phase was washed with brine. Aqueous phases were back extracted with ethyl acetate (15 mL) and the combined organic extracts were dried (MgSO₄) and evaporated to afford the epoxide (117 mg, 70%) as a white solid which gave an ¹H NMR spectrum consistent with structure 1 prepared by literature method.

### Example 38

Alcohol 51: To a solution of protected alcohol (PG=methoxymethyl) (342 mg, 1.15 mmol) in CH₂Cl₂ (10 mL) at 0° C. was added trifluoroacetic acid (8 mL). After 5 min at 0° C., the solution was stirred 1 h at room temperature and was evaporated. The crude product was purified on silica gel (ethyl acetate) to afford alcohol 51 (237 mg, 82%) as an oil: ¹H NMR (300 MHz, CDCl₃) δ 2.11 (s, 3H), 2.45 (m, 1H), 2.97 (dd, 1H, J=3.8, 18.8), 3.66 (m, 2H), 3.78 (s, 3H), 4.40 (br s, 1H), 5.22 (br s, 1H), 6.19 (br s, 1H), 6.82 (m, 1H).

### Example 39

Methyl ether 150: To a solution of alcohol 51 (46 mg, 0.18 mmol) and methyl iodide (56 μL, 0.90 mmol) in THF (0.7 mL) at 0° C. was added NaH as a 60% mineral oil dispersion (8 mg, 0.20 mmol). The solution was stirred at 0° C. for 2.5 h, and a second portion of NaH (2 mg) was added. After an additional 1 h at 0° C. and 4 h at room temperature the solution was cooled to 0° C. and 5% citric acid (0.5 mL) was added. The mixture was extracted with ethyl acetate (4×2 mL) and the combined organic extracts were dried (MgSO₄), and evaporated. Purification of the crude residue on silica gel (ethyl acetate) gave methyl ether 150 (12 mg, 25%) as a solid: ¹H NMR (300 MHz, CDCl₃) δ 2.07 (s, 3H), 2.23-2.34 (m, 1H), 2.89 (app ddd, 1H), 3.43 (s, 3H), 3.58 (m, 1H), 3.78 (s, 3H), 4.13 (m, 1H), 4.40 (m, 1H), 5.73 (d, 1H, J=7.6), 6.89 (m, 1H).

### Example 40

Amino acid 151: To a solution of methyl ether 150 (12 mg, 0.45 mmol) in THF(1 mL)/water (100 μL) was added polymer support Ph₃P (75 mg, 3 mmol P/g resin). The mixture was stirred at room temperature for 19 h. The resin was filtered, washed several times with THF and the combined filtrate and washings were evaporated to provide 8 mg of a crude residue. The residue was dissolved in THF (0.5 mL), and 0.5 M KOH (132 μL)/water (250 μL) was added. The solution was stirred at room temperature for 1.25 h and the pH was adjusted to 3-4 with IR120 ion exchange resin. The resin was filtered and was stirred with 1M HCl. After filtration, the resin was subjected to the same treatment with 1M HCl until the acidic washes no longer tested positive for amine with ninhydrin. The combined resin washings were evaporated and the residue was purified on C-18 reverse phase silica eluting with water to afford after lyophilization, amino acid 151 (1.8 mg, 15%) as a white solid: ¹H NMR (300 MHz, D₂O) δ 2.09 (s, 3H), 2.48-2.59 (app qt, 1H), 2.94 (dd, 1H, J=5.7, 17.4), 3.61 (m, 1H), 4.14-4.26 (m, 2H), 6.86 (br s, 1H).

### Example 41

Amino acid allyl ether 153: To a solution of azide 6 (16 mg, 0.054 mmol) in THF (0.50 mL) and H₂O (35 μL) was added polystyrene supported PPh₃ (50 mg). The reaction was stirred at ambient temperature for 24 h, filtered through a sintered glass funnel and washed with hot methanol. Concentration in vacuo gave the crude amino ester which was dissolved in THF (1.0 mL) and treated with aqueous KOH (220 μL of a 0.5 M solution). After stirring at ambient temperature for 2 h Amberlite IR-120 (plus) acidic resin was added until the solution attained pH=4.5. The resin was filtered and washed with ethanol and H₂O. Concentration in vacuo gave a pale orange solid which was purified by reverse phase C₁₈ chromatography using H₂O as an eluent. Fractions containing the desired product were pooled and lyophilized to give the amino acid as a white powder. ¹H NMR (D₂O, 300 MHz): δ 6.51 (br t, 1H); 6.05-5.80 (m, 1H, —CH═, allyl); 5.36-5.24 (m, 2H, ═CH₂, allyl); 4.35-4.25 (m, 1H); 4.25-4.05 (m, 2H, —CH₂—, allyl); 4.02-3.95 (m, 1H); 3.81-3.70 (m, 1H); 2.86-2.77 (apparent dd, 1H); 2.35-2.24 (complex m, 1H); 2.09 (s, 3H).

### Example 42

Epoxide 161: MCPBA (690 mg) was added to a solution of olefin 160 (532 mg, 1.61 mmol, prepared by Example 14, crude mesylate was filtered through silica gel using 30% EtOAc/Hexanes prior to use) in dichloromethane (15 mL) cooled to 0° C. The mixture was warmed to room temperature and stirred overnight. The bulk of the solvent was removed under vacuum and the mixture diluted with ethyl acetate. The organic layer was washed with aqueous sodium bisulfite, saturated sodium bicarbonate, brine and dried over MgSO₄. Concentration in vacuo followed by flash column chromatography of the residue (30% hexanes in ethyl acetate) gave 437 mg (78%) of 161 as a pale oil. ¹H NMR (CDCl₃, 300 MHz): [1:1 mixture of diastereomers] δ [4.75 (dd, J=3.9, 8.2 Hz) & 4.71 (dd, J=3.9, 8.4 Hz), 1H total]; 4.37 (m, 1H); 4.25-4.00 (m, 2H); 3.78 (s, 3H); [3.68 (dd, J=5.7, 11.7 Hz) & 3.51 (dd, J=6.6, 11.7 Hz), 1H total]; [3.17 (s) & 3.16 (s), 3H total]; [2.99 (m) & 2.93 (m), 1H total]; [2.83 (t, J=4.1 Hz) & 2.82 (t, J=4.5 Hz), 1H total]; 2.70-2.60 (m, 1H); 2.45-2.30 (m, 1H).

### Example 43

Diol 162: The epoxide 161 (437 mg, 1.23 mmol) was gently reluxed for 1 h in THF (20 mL) and H₂O (10 mL) containing 5 drops of 70% HClO₄. Solid NaHCO₃ was added and the mixture concentrated in vacuo. The residue was dissolved in EtOAc, washed with brine and dried. Concentration in vacuo gave the crude diol 162 as a pale oil in quantitative yield. Used without any purification for the next reaction.

### Example 44

Aldehyde 163: Oxidation of diol 162 was carried out according to the procedure of Vo-Quang and co-workers, “Synthesis”, 68 (1988). To a slurry of silica gel (4.3 g) in dichloromethane (30 mL) was added a solution of NaIO₄ (4.4 mL of a 0.65 M aqueous solution). To this slurry was added a solution of the crude diol 162 (520 mg) in EtOAc (5 mL) and dichloromethane (15 mL). After 1 h the solids were filtered and washed with 20% hexanes/EtOAc. Concentration gave an oily residue which was dissolved in EtOAc and dried over MgSO₄. Concentration in vacuo gave the aldehyde 163 as a pale oil which was used immediately for the next reaction. ¹H NMR (CDCl₃, 300 MHz): δ 9.69 (s, 1H); 6.98 (m, 1H); 4.72 (dd, 1H, J=3.7, 9.1 Hz); 4.53 (d, 1H, J=18.3 Hz); 4.45 (d, 1H, J=18.3 Hz); 4.31 (m, 1H); 4.26-4.18 (m, 1H); 3.79 (s, 3H); 3.19 (s, 3H); 3.05 (dd, 1H, J=5.7, 18.6 Hz); 2.20-2.45 (m, 1H).

### Example 45

Alcohol 164: The crude aldehyde 163 was treated with NaCNBH₃ according to the procedure of Borch and co-workers, “J. Amer. Chem. Soc.”, 93:2897 (1971) to give 269 mg (65%) of the alcohol 164 after flash chromatography (40% hexanes in ethyl acetate). ¹H NMR (CDCl₃, 300 MHz): δ 6.91 (m, 1H); 4.75 (dd, 1H, J=3.9, 8.7 Hz); 4.34 (br t, 1H, J=4.1 Hz); 4.25-4.15 (m, 1H); 3.85-3.70 (m, 4H); 3.77 (s, 3H); 3.16 (s, 3H); 2.95 (dd, 1H, J=5.7, 18.6 Hz); 2.37 (dd, 1H, J=7.1, 18.6 Hz); 2.26 (br s, 1H).

### Example 46

Aziridine 165: The alcohol 164 (208 mg, 0.62 mmol) was acetylated in the usual manner (AcCl, pyridine, dichloromethane, cat. DMAP) to give the acetate (241 mg, 100%). The crude acetate (202 mg, 0.54 mmol) was treated at room temperature with Ph₃P (155 mg) in THF (12 mL) for 2 h. H₂O (1.1 mL) and triethylamine (224 μL) were then added and the solution stirred overnight. The reaction mixture was concentrated and the residue partitioned between ethyl acetate and saturated bicarbonate/brine. The organic layer was dried, concentrated in vacuo and purified by flash chromatography (10% MeOH in EtOAc) to give 125 mg (90%) of aziridine 165 as a white solid. ¹H NMR (CDCl₃, 300 MHz): δ 6.80 (m, 1H); 4.44 (br s, 1H); 4.23 (t, 2H, J=4.8 Hz); 3.82-3.65 (m, 2H); 3.74 (s, 3H); 2.85 (br d, 1H, J=19.2 Hz); 2.65-2.40 (m, 3H); 2.09 (s, 3H); 1.25 (br s, 1H).

### Example 47

N-Boc aziridine 166: Boc anhydride (113 mg, 0.52 mmol) was added to a solution of aziridine 165 (125 mg, 0.49 mmol), triethylamine (70 μL), DMAP (cat. amount) in dichloromethane (7 mL). After 1 h the reaction was concentrated and the residue subjected to flash chromatography (40% EtoAc in hexanes) to give 154 mg (88%) of the N Boc aziridine 166 as a pale oil. ¹H NMR (CDCl₃, 300 MHz): δ 6.82 (m, 1H); 4.47 (br m, 1H); 4.23 (t, 2H, J=4.7 Hz); 3.81 (t, 2H, J=4.7 Hz); 3.75 (s, 3H); 3.00 (br d, 1H, J=18.0 Hz); 2.90-2.85 (m, 2H); 2.65-2.55 (m, 1H); 2.10 (s, 3H); 1.44 (s, 9H).

### Example 48

Azido ester 167: Aziridine 166 (154 mg, 0.43 mmol), sodium azide (216 mg), and ammonium chloride (223 mg) was heated at 100° C. in DMF (5 mL) for 18 h. The cooled reaction mixture was partitioned between ethyl ether and brine. The ether layer was washed with H₂O, brine and dried over MgSO₄. Concentration gave a crude residue which was treated with 40% TFA in dichloromethane at room temperature. After 2 h the reaction was concentrated in vacuo to give a pale oil which was passed through a short column of silica gel eluting with EtOAc. The product was then acylated in the usual manner (AcCl, pyridine, dichloromethane, cat. DMAP) to give the azido ester 167 as a pale yellow oil 16 mg (11% for 3 steps) after flash chromatography (5% MeOH in chloroform). ¹H NMR (CDCl₃, 300 MHz): δ 6.85 (m, 1H); 5.80 (br d, 1H, J=7.8 Hz); 4.55 (m, 1H); 4.25-4.10 (m, 3H); 3.90-3.85 (m, 2H); 3.78 (s, 3H); 3.55 (m, 1H); 2.90 (dd, 1H, J=5.4, 17.0 Hz); 2.45-2.25 (m, 1H); 2.10 (s, 3H); 2.05 (s, 3H).

### Example 49

Amino acid 168: To a solution of ester 167 (16 mg, 0.047 mmol) in THF (1 mL) cooled to 0° C. was added aq. KOH (208 μl of a 0.476 M solution). The reaction was then warmed to room temperature and stirred for 2 h. The reaction was then acidified to pH=4.0 with Amberlite IR-120 (plus) acidic resin. The resin was then filtered and washed with ethanol and H₂O. Concentration in vacuo gave a 14 mg (100%) of the azido carboxylic acid as a white solid. The azido acid was dissolved in ethanol (2 mL) and treated with hydrogen gas (1 atm) over Lindlar's catalyst (15 mg) for 16 h according to the procedure of Corey and co-workers, “Synthesis”, 590 (1975). The reaction mixture was filtered through a celite pad and washed with hot ethanol and H₂O. Concentration in vacuo gave a pale orange solid which was purified by a C₁₈ column chromatography eluting with H₂O. The fractions containing the product were pooled and lyophilzed to give 9.8 mg of 168 as a white powder. ¹H NMR (D₂O, 500 MHz): δ: 6.53 (br s, 1H); 4.28 (br m, 1H); 4.08 (dd, 1H, J=11.0, 11.0 Hz); 3.80-3.65 (complex m, 4H); 3.44 (m, 1H); 2.84 (apparent dd, 1H); 2.46-2.39 (complex m, 1H); 2.08 (s, 3H).

### Example 50

Epoxy MOM ether 19 (PG=methoxymethyl): Prepared in 74% from epoxy alcohol 1 according to the procedure of Mordini and co-workers, “J. Org. Chem.”, 59:4784 (1994). ¹H NMR (CDCl₃, 300 MHz): δ 6.73 (m, 1H); 4.87 (s, 2H); 4.59 (t, 1H, J=2.4 Hz); 3.76 (s, 3H); 3.57 (m, 1H); 3.50-3.40 (m, 1H); 3.48 (s, 3H); 3.10(d, J=19.5 Hz); 2.45 (m, 1H).

### Example 51

Aziridine 170: Prepared in 77% overall from epoxide 19 (PG=methoxymethyl) according to the general protocol described in Examples 3 and 4: ¹H NMR (CDCl₃, 300 MHz): δ 6.85 (m, 1H); 4.78 (s, 2H); 4.54 (m, 1H); 3.73 (s, 3H); 3.41 (s, 3H); 2.87 (d, 1H, J=18.9 Hz); 2.70-2.45 (m, 3H).

### Example 52

Azido ester 22 (PG=methoxymethyl): The aziridine 170 (329 mg, 1.54 mmol), NaN₃ (446 mg) and NH₄Cl (151 mg) was heated at 65° C. in DMF (20 mL) for 18 h. The cooled reaction mixture was partitioned between ethyl ether and brine. The ether layer was washed with H₂O, brine and dried over MgSO₄. Concentration in vacuo gave the crude azido amine as a pale oil which was taken up in CH₂Cl₂ (15 mL) and treated with pyridine (4 mL) and AcCl (150 μL). Aqueous work up followed by flash chromatography of the residue gave 350 mg (76%) of azido ester 22 (PG=methoxymethyl) as a pale oil. ¹H NMR (CDCl₃, 300 MHz): δ 6.78 (s, 1H); 6.39 (br d, 1H, J=7.8 Hz); 4.72 (d, 1H, J=6.9 Hz); 4.66 (d, 1H, J=6.9 Hz); 4.53 (br d, 1H, J=8.4 Hz); 4.00-3.90 (m, 1H); 3.80-3.65 (m, 1H); 3.75 (s, 3H); 3.37 (s, 3H); 2.85 (dd, 1H, J=5.4, 17.7 Hz); 2.35-2.20 (m, 1H); 2.04 (s, 3H).

### Example 53

Amino acid 114: The azide 22 (PG=methoxymethyl) (39 mg, 0.131 mmol) was treated with hydrogen gas at 1 atmosphere over Lindlar's catalyst (39 mg) in ethanol for 2.5 h according to the procedure of Corey and co-workers, “Synthesis”, 590 (1975). The reaction mixture was filtered through a celite pad, washed with hot ethanol and concentrated to give the crude amine 33 mg (92%) as a pale foam. The amine in THF (1 mL) was treated with aq. KOH (380 μL of a 0.476 M solution). After 1 h the reaction was acidified to pH=4.0 with Amberlite IR-120 (plus) acidic resin. The resin was then filtered, washed with H₂O and concentrated to give a pale solid which was purified by a C₁₈ column chromatography eluting with H₂O. The fractions containing the product were pooled and lyophilzed to give 20 mg of 114 as a white powder. ¹H NMR (D₂O, 300 MHz): δ 6.65 (s, 1H); 4.87 (d, 1H, J=7.5 Hz); 4.76 (d, 1H, J=7.5 Hz); 4.47 (br d, 1H, J=8.7 Hz); 4.16 (dd, 1H, J=11.4, 11.4 Hz); 3.70-3.55 (m, 1H); 3.43 (s, 3H); 2.95 (dd, 1H, J=5.7, 17.4 Hz); 2.60-2.45 (m, 1H); 2.11 (s, 3H).

### Example 54

Amino acid 171: To solid amino acid 114 (4 mg, 0.015 mmol) was added 40% TFA in CH₂Cl₂ (1 mL, cooled to 0° C. prior to addition). After stirring at room temperature for 1.5 h the reaction mixture was concentrated to give a white foam. Co-evaporation from H₂O several times followed by lyophilization gave a white solid, 5.5 mg of 117 as the TFA salt. ¹H NMR (D₂O, 300 MHz): δ 6.85 (m, 1H); 4.45 (m, 1H); 4.05 (dd, 1H, J=11.4, 11.4 Hz); 3.65-3.55 (m, 1H); 3.00-2.90 (m, 1H); 2.60-2.45 (m, 1H); 2.09 (s, 3H).

### Example 55

Acetonide 180: To a suspension of shikimic acid (25 g, 144 mmol, Aldrich) in methanol (300 mL) was added p-toluenesulfonic acid (274 mg, 1.44 mmol, 1 mol %) and the mixture was heated to reflux for 2 h. After adding more p-toluenesulfonic acid (1 mol %) the reaction was refluxed for 26 h and was evaporated. The crude methyl ester (28.17 g) was suspended in acetone (300 mL) and was treated with dimethoxypropane (35 mL, 288 mmol) and was stirred at room temperature for 6 h and then was evaporated. The crude product was dissolved in ethyl acetate (400 mL) and was washed with saturated NaHCO₃ (3×125 mL) and saturated NaCl. The organic phase was dried (MgSO₄), filtered, and evaporated to afford crude acetonide 180 (˜29.4 g) which was used directly: ¹H NMR (CDCl₃) δ 6.91 (t, 1H, J=1.1), 4.74 (t, 1H, J=4.8), 4.11 (t, 1H, J=6.9), 3.90 (m, 1H), 2.79 (dd, 1H, J=4.5, 17.4), 2.25 (m, 2H), 1.44 (s, 3H), 1.40 (s, 3H).

### Example 56

Mesylate 130: To a solution of acetonide 180 (29.4 g, 141 mmol) in CH₂Cl₂, (250 mL) at 0° C. was added triethylamine (29.5 mL, 212 mmol) followed by the addition of methanesulfonyl chloride (13.6 mL, 176 mmol) over a period of 10 min. The reaction was stirred at 0° C. for 1 h and ice cold water (250 mL) was added. After transfer to a separatory funnel, the organic phase was washed with water, 5% citric acid (300 mL), saturated NaHCO₃ (300 mL) and was dried (MgSO₄), filtered, and evaporated. The crude product was filtered through a short plug of silica gel on a fritted glass funnel eluting with ethyl acetate. The filtrate was evaporated to afford mesylate 130 (39.5 g, 91%) as a viscous oil which was used directly in the next step: ¹H NMR (CDCl₃) δ 6.96 (m, 1H), 4.80 (m, 2H), 4.28 (dd, 1H, J=6.6, 7.5), 3.79 (s, 3H), 3.12 (s, 3H), 3.01 (dd, 1H, J=5, 17.7), 2.56-2.46 (m, 1H).

### Example 57

Diol 131: To a solution of mesylate 130 (35.85 g, 117 mmol) in methanol (500 mL) was added p-toluenesulfonic acid (1.11 g, 5.85 mmol, 5 mol %) and the solution was refluxed for 1.5 h and was evaporated. The residue was redissolved in methanol (500 mL) and was refluxed an additional 4 h. The solvent was evaporated and the crude oil was triturated with diethyl ether (250 mL). After completing the crystallization overnight at 0° C., the solid was filtered and was washed with cold diethyl ether, and dried to afford diol 131 (24.76 g) as a white solid. Evaporation of the filtrate and crystallization of the residue from methanol/diethyl ether gave an additional 1.55 g. Obtained 26.3 g (85%) of diol 131: ¹H NMR (CD₃OD) δ 6.83 (m, 1H), 4.86 (m, 1H), 4.37 (t, 1H, J=4.2), 3.87 (dd, 1H, J=4.2, 8.4), 3.75 (s, 3H), 3.13 (s, 3H), 2.98-2.90 (m, 1H), 2.53-2.43 (m, 1H).

### Example 58

Epoxy alcohol 1: A suspension of diol 131 (20.78 g, 78 mmol) in tetrahydrofuran (400 mL) at 0° C. was treated with 1, 8-diazabicyclo[5.4.0]undec-7-ene (11.7 mL, 78 mmol) and was stirred at room temperature for 9 h at which time the reaction was complete. The reaction was evaporated and the crude residue was dissolved in CH₂Cl₂ (200 mL) and was washed with saturated NaCl (300 mL). The aqueous phase was extracted with CH₂Cl₂ (2×200 mL). The combined organic extracts were dried (MgSO₄), filtered, and evaporated. The crude product was purified on silica gel (ethyl acetate) to afford epoxy alcohol 1 (12 g, 90%) as a white solid whose ¹H NMR spectrum was consistent with that reported in the literature: McGowan, D. A.; Berchtold, G. A., “J. Org. Chem.”, 46:2381 (1981).

### Example 59

Methoxymethyl ether 19 (PG=methoxymethyl): To a solution of epoxy alcohol 1 (4 g, 23.5 mmol) in CH₂Cl₂ (100 mL) was added N,N′-diisopropylethylamine (12.3 mL, 70.5 mmol) followed by chloromethyl methyl ether (3.6 mL, 47 mmol, distilled from tech. grade). The solution was refluxed for 3.5 h and the solvent was evaporated. The residue was partitioned between ethyl acetate (200 mL) and water (200 mL). The aqueous phase was extracted with ethyl acetate (100 mL). The combined organic extracts were washed with saturated NaCl (100 mL), dried (MgSO₄), filtered, and evaporated to afford 4.9 g of a solid residue which was of suitable purity to use directly in the next step: mp 62-65° (crude); mp 64-66° C. (diethyl ether/hexane); ¹H NMR (CDCl₃) δ 6.73 (m, 1H), 4.87 (s, 2H), 4.59 (m, 1H), 3.75 (s, 3H), 3.57 (m, 1H), 3.48 (m overlapping s, 4H), 3.07 (dd, 1H, J=1.2, 19.8), 2.47 (dq, 1H, J=2.7, 19.5). Ethyl Ester Analog of Compound 19: To a solution of the corresponding ethyl ester of compound 1 (12.0 g, 0.065 mol) in CH₂Cl₂ (277 mL) at room temperature was added diisopropylethyl amine (34.0 mL, 0.13 mol) followed by chloromethyl methyl ether (10.0 mL, 0.19 mol). The reaction mixture was then gently refluxed for 2 h, cooled, concentrated in vacuo, and partitioned between EtOAc and water. The organic layer was separated and washed successively with dil. HCl, saturated bicarb, brine and dried over MgSO₄. Concentration in vacuo followed by flash chromatography on silica gel (50% hexanes in EtOAc) gave 13.3 g (90%) of the corresponding ethyl ester of compound 19 as a colorless liquid. ¹H NMR(300 MHz, CDCl₃) 86.73-6.71 (m, 1H); 4.87 (s, 2H); 4.61-4.57 (m, 1H); 4.21 (q, 2H, J=7.2 Hz); 3.60-3.55 (m, 1H); 3.50-3.45 (m, 1H); 3.48 (s, 3H); 3.12-3.05 (m, 1H); 2.52-2.42 (m, 1H); 1.29 (t, 3H, J=7.2 Hz).

### Example 60

Alcohol 181: To a solution of methoxymethyl ether 19 (PG=methoxymethyl) (4.9 g, 22.9 mmol) in 8/1-MeOH/H₂O (175 mL, v/v) was added sodium azide (7.44 g, 114.5 mmol) and ammonium chloride (2.69 g, 50.4 mmol) and the mixture was refluxed for 15 h. The reaction was diluted with water (75 mL) to dissolve precipitated salts and the solution was concentrated to remove methanol. The resulting aqueous phase containing a precipitated oily residue was diluted to a volume of 200 mL with water and was extracted with ethyl acetate (3×100 mL). The combined organic extracts were washed with saturated NaCl (100 mL), dried (MgSO₄), filtered and evaporated. The crude was purified on silica gel (1/1-hexane/ethyl acetate) to afford alcohol 181 (5.09 g, 86%) as a pale yellow oil. Subsequent preparations of alcohol 181 provided material which was of sufficient purity to use in the next step without further purification: ¹H NMR (CDCl₃) δ 6.86 (m, 1H), 4.79 (s, 2H), 4.31 (br t, 1H, J=4.2), 3.90-3.75, 3.77 (m overlapping s, 5H), 3.43 (s, 3H), 2.92 (d, 1H, J=6.6), 2.87 (dd, 1H, J=5.4, 18.6), 2.21-2.30 (m, 1H).

### Example 61

Mesylate 184: To a solution of alcohol 181 (6.47 g, 25.2 mmol) in CH₂Cl₂ (100 mL) at 0° C. was added first triethyl amine (4.4 mL, 31.5 mmol) then methanesulfonyl chloride (2.14 mL, 27.7 mmol). The reaction was stirred at 0° C. for 45 min then was warmed to room temperature stirring for 15 min. The reaction was evaporated and the residue was partitioned between ethyl acetate (200 mL) and water (100 mL). The organic phase was washed with water (100 mL), saturated NaHCO₃ (100 mL), saturated NaCl (100 mL). The water washes were extracted with a single portion of ethyl acetate which was washed with the same NaHCO₃/NaCl solutions. The combined organic extracts were dried (MgSO₄), filtered, and evaporated. The crude product was of suitable purity to be used directly in the next step: ¹H NMR (CDCl₃) δ 6.85 (m, 1H), 4.82 (d, 1H, J=6.9), 4.73 (d, 1H, J=6.9), 4.67 (dd, 1H, J=3.9, 9.0), 4.53 (br t, 1H, J=4.2), 3.78 (s, 3H), 3.41 (s, 3H), 3.15 (s, 3H), 2.98 (dd, 1H, J=6.0, 18.6), 2.37 (m, 1H); ¹³C NMR (CDCl₃) δ 165.6, 134.3, 129.6, 96.5, 78.4, 69.6, 55.8, 55.7, 52.1, 38.2, 29.1.

### Example 62

Aziridine 170: To a solution of mesylate 184 (8.56 g, 25 mmol) in THF (150 mL) at 0° C. was added Ph₃P (8.2 g, 31 mmol), initially adding a third of the amount while cooling and then after removing the ice bath adding the remainder of the Ph₃P over a period of 10-15 min. After complete addition of the Ph₃P the reactionwas stirred at room temperature for 3 h with the formation of a white precipitate. To this suspension was added triethyl amine (5.2 mL, 37.5 mmol) and water (10 mL) and the mixture was stirred at room temperature for 12 h. The reaction was concentrated to remove THF and the residue was partitioned between CH₂Cl₂ (200 mL) and saturated NaCl (200 mL). The aqueous phase was extracted with several portions of CH₂Cl₂ and the combined organic extracts were dried (Na₂SO₄), filtered, and evaporated to afford a crude product which was purified on silica gel (10% MeOH/EtOAc) to afford aziridine 170 (4.18 g, 78%) as an oil which typically contained trace amounts of triphenylphosphine oxide impurity: ¹H NMR (CDCl₃) δ 6.81 (m, 1H), 4.78 (s, 2H), 4.54 (m, 1H), 3.73 (s, 3H), 3.41 (s, 3H), 2.87 (app dd, 1H), 2.64 (br s, 1H), 2.56-2.47 (m, 2H), NH signal was not apparent; ¹³C NMR (CDCl₃) δ 166.9, 132.5, 128.0, 95.9, 69.5, 55.2, 51.6, 31.1, 27.7, 24.1.

### Example 63

Amine 182: To a solution of aziridine 170 (3.2 g, 15 mmol) in DMF (30 mL) was applied a vacuum on a rotary evaporator (40° C.) for several minutes to degas the solution. To the solution was added sodium azide (4.9 g, 75 mmol) and ammonium chloride (1.6 g, 30 mmol) and the mixture was heated at 65-70° C. for 21 h. The reaction mixture was cooled to room temperature, diluted with ethyl acetate (˜100 mL) and was filtered. The filtrate was evaporated and the residue was partitioned between diethyl ether (100 mL) and saturated NaCl (100 mL). The organic phase was washed again with saturated NaCl (100 mL), dried (MgSO₄), filtered, and was evaporated. Additional crude product was obtained from the aqueous washings by extraction with ethyl acetate and treated in the same manner as described above. The crude product was purified on silica gel (5%MeOH/CH₂Cl₂) to afford amine 182 (2.95 g) as an oil which contained a small amount of triphenylphosphine oxide impurity from the previous step: ¹H NMR (CDCl₃) δ 6.82 (t, 1H, J=2.3), 4.81 (d, 1H, J=7.2), 4.77 (d, 1H, J=6.9), 4.09-4.04 (m, 1H), 3.76 (s, 3H), 3.47 and 3.44 (m overlapping s, 4H), 2.94-2.86 (m, 2H), 2.36-2.24 (m, 1H); ¹³C NMR (CDCl₃) δ 165.9, 137.3, 128.2, 96.5, 79.3, 61.5, 55.7, 55.6, 51.9, 29.5.

### Example 64

N-Trityl aziridine 183: Amine 182 (2.59 g, 10.2 mmol) was dissolved in 5% HCl/MeOH (30 mL) and the solution was stirred for 3 h at room temperature. Additional 5% HCl/MeOH (10 mL) was added stirring 1 h and the solvent was evaporated to afford 2.52 g of the HCl salt as a tan solid after high vacuum. To a suspension of the HCl salt in CH₂Cl₂ (50 mL) at 0° C. was added triethylamine (3.55 mL, 25.5 mmol) followed by the addition of solid trityl chloride (5.55 g, 12.8 mmol) in one portion. The mixture was stirred at 0° C. for 1 h and then was warmed to room temperature stirring for 2 h. The reaction was cooled to 0° C., triethylamine (3.6 mL, 25.5 mmol) was added and methane sulfonyl chloride (0.97 mL, 12.5 mmol) was added, stirring the resulting mixture for 1 h at 0° C. and for 22 h at room temperature. The reaction was evaporated and the residue was partitioned between diethyl ether (200 mL) and water (200 mL). The organic phase was washed with water (200 mL) and the combined aqueous phases were extracted with diethyl ether (200 mL). The combined organic extracts were washed with water (100 mL), saturated NaCl (200 mL) and were dried (Na₂SO₄), filtered, and evaporated. The crude product was purified on silica gel (1/1-hexane/CH₂Cl₂) to afford N-trityl aziridine 183 (3.84 g, 86%) as a white foam: ¹H NMR (CDCl₃) δ 7.4-7.23 (m, 16H), 4.32 (m, 1H), 3.81 (s, 3H), 3.06 (dt, 1H, J=1.8, 17.1), 2.94-2.86 (m, 1H), 2.12 (m, 1H), 1.85 (t, 1H, J=5.0).

### Example 65

Compound 190: A solution of N-trityl aziridine 183 (100 mg, 0.23 mmol), cyclohexanol (2 mL) and boron trifluoride etherate (42 μL, 0.35 mmol) was heated at 70° C. for 1.25 h and was evaporated. The residue was dissolved in pyridine (2 mL) and was treated with acetic anhydride (110 μL, 1.15 mmol) and catalytic DMAP. After stirring for 3 h at room temperature the reaction was evaporated. The residue was partitioned between ethyl acetate and 5% citric acid. The aqueous phase was extracted with ethyl acetate and the combined organic extracts were washed with saturated NaHCO₃, and saturated NaCl. The organic phase was dried (MgSO₄), filtered, and evaporated. The crude product was purified on silica gel (1/1-hexane/ethyl acetate) to afford compound 190 (53 mg, 69%) as a solid: mp 105-107° C. (ethyl acetate/hexane); ¹H NMR (CDCl₃) δ 6.78 (m, 1H), 6.11 (d, 1H, J=7.4), 4.61 (m, 1H), 4.32-4.23 (m, 1H), 3.76 (s, 3H), 3.44-3.28 (m, 2H), 2.85 (dd, 1H, J=5.7, 17.6), 2.28-2.17 (m, 1H), 2.04 (s, 3H), 1.88-1.19 (m, 10H).

### Example 66

Compound 191: To a solution of compound 190 (49 mg, 0.15 mmol) in THF was added triphenylphosphine (57 mg, 0.22 mmol) and water (270 μL) and the solution was heated at 50° C. for 10 h. The reaction was evaporated and the residue was dissolved in ethyl acetate, dried (Na₂SO₄), filtered and evaporated. The crude product was purified on silica gel (1/1-methanol/ethyl acetate) to afford the amine (46 mg) as a pale yellow solid. The a solution of the amine in THF (1.5 mL) was added 1.039N KOH solution (217 μL) and water (200 μL). The mixture was stirred at room temperature for 1 h and was then cooled to 0° C. and acidified to pH 6-6.5 with IR 120 ion exchange resin. The resin was filtered, washed with methanol and the filtrate was evaporated. The solid residue was dissolved in water and was passed through a column (4×1 cm) of C-18 reverse phase silica gel eluting with water and then 2.5% acetonitrile/water. Product fractions were combined and evaporated and the residue was dissolved in water and lyophilized to afford amino acid 191 (28 mg) as a white solid: ¹H NMR (D₂O) δ 6.47 (br s, 1H), 4.80 (br d, 1H), 4.00 (dd, 1H, J=8.9, 11.6), 3.59-3.50 (m, 2H), 2.87 (dd, 1H, J=5.5, 17.2), 2.06 (s, 3H), 1.90-1.15 (series of m, 10H); Anal. Calcd for C₁₅H₂₄N₂O₄.H₂O: C, 57.31; H, 8.34; N, 8.91. Found: C, 57.38; H, 8.09; N, 8.77.

### Example 67

bis-Boc guanidino ester 201: Treated according to the procedure of Kim and Qian, “Tetrahedron Lett.”, 34:7677 (1993). To a solution of amine 200 (529 mg, 1.97 mmol, prepared by the method of Example 109, bis-Boc thiourea (561 mg, 2.02 mmol) and Et₃N (930 μL) in dry DMF (5.0 mL) cooled to 0° C. was added HgCl₂ (593 mg, 2.18 mmol) in one portion. The heterogeneous reaction mixture was stirred for 45 min at 0° C. and then at room temperature for 15 min, after which the reaction was diluted with EtOAc and filtered through a pad of celite. Concentration in vacuo followed by flash chromatography of the residue on silica gel (10% hexanes in ethyl acetate) gave 904 mg (90%) of 201 as a pale oil. ¹H NMR (CDCl₃, 300 MHz): δ 11.39 (s, 1H); 8.63 (d, 1H, J=7.8 Hz); 6.89 (t, 1H, J=2.4 Hz); 6.46 (d, 1H, J=8.7 Hz); 4.43-4.32 (m, 1H); 4.27-4.17 (m, 1H); 4.13-4.06 (m, 1H); 3.77 (s, 3H); 3.67-3.59 (m, 1H); 2.83 (dd, 1H, J=5.1, 17.7 Hz); 2.45-2.33 (m, 1H); 1.95 (s, 3H); 1.65-1.50 (m, 2H); 1.45 (s, 18H); 0.90 (t, 3H, J=7.5 Hz).

### Example 68

Carboxylic acid 202: To a solution of methyl ester 201 (904 mg, 1.77 mmol) in THF (10 mL) was added aqueous KOH (3.45 mL of a 1.039 N solution). The reaction mixture was stirred at room temperature for 17 h, cooled to 0° C. and acidified to pH 4.0 with Amberlite IR-120 (H⁺) acidic resin. The resin was filtered and washed with water and methanol. Concentration in vacuo gave the free acid as a pale foam which was used without further purification in the next reaction.

### Example 69

Guanidine carboxylic acid 203: To a solution of bis-Boc guanidnyl acid 202 (crude from previous reaction) in CH₂Cl₂ (40 mL) cooled to 0° C. was added neat trifluoroacetic acid (25 mL). The reaction mixture was stirred at 0° C. for 1 h and then at room temperature for 2 h. Concentration in vacuo gave a pale orange solid which was purified by C₁₈ reverse phase chromatography eluting with water. Fractions containing the desired product were pooled and lyophilized to give 495 mg (68%, 2 steps) of the guanidine carboxylic acid 203 as the trifluoroacetic acid salt. ¹H NMR (D₂O, 300 MHz): δ 6.66 (s, 1H); 4.29 (bd, 1H, J=9.0 Hz); 4.01 (dd, 1H, J=10.8, 10.8 Hz); 3.87-3.79 (m, 1H); 3.76-3.67 (m, 1H); 3.60-3.50 (m, 1H); 2.83 (dd, 1H, J=5.1, 17.4 Hz); 2.47-2.36 (m, 1H); 2.06 (s, 3H); 1.65-1.50 (m, 2H); 0.90 (t, 3H, J=7.2 Hz). Anal. Calcd for C₁₅H₂₃O₆N₄F₃: C, 43.69; H, 5.62; N, 13.59. Found: C, 43.29; H, 5.90; N, 13.78.

### Example 70

Formamidine carboxylic acid 204: A solution of amino acid 102 (25 mg, 0.10 mmol, prepared by the method of Example 110) in water (500 μL) at 0-5° C. was adjusted to pH 8.5 with 1.0 N NaOH. Benzyl formimidate hydrochloride (45 mg, 0.26 mmol) was added in one portion and the reaction mixture was stirred for 3 h at this temperature while maintaining the pH at 8.5-9.0 with 1.0 N NaOH. The reaction was then concentrated in vacuo and purified by C₁₈ reverse phase chromatography eluting with water. Fractions containing the desired product were pooled and lyophilized to give 4.0 mg (13%) of the formamidine carboxylic acid 204. ¹H NMR (D₂O, 300 MHz): δ 7.85 (s, 1H); 6.53 (bd, 1H, J=7.8 Hz); 4.32-4.25 (bm, 1H); 4.10-3.97 (m, 1H); 3.76-3.67 (m, 2H); 3.57-3.49 (m, 1H); 2.86-2.81 (m, 1H); 2.55-2.40 (m, 1H); 2.04 (s, 3H); 1.66-1.50 (m, 2H); 0.90 (t, 3H, J=7.4 Hz).

### Example 71

Amino acid 206: To a solution of amino methyl ester 205 (84 mg, 0.331 mmol, prepared by Example 107) in THF (1.0 mL) was added aqueous KOH (481 μL of a 1.039 N solution). The reaction mixture was stirred at room temperature for 2.5 h and acidified to pH 6.5 with Amberlite IR-120 (H⁺) acidic resin. The resin was filtered and washed with water and methanol. Concentration in vacuo gave the amino acid as a white solid which was purified by C₁₈ reverse phase chromatography eluting with water. Fractions containing the desired product were pooled and lyophilized to give 59 mg (74%) of the amino acid 206. ¹H NMR (CD₃OD, 300 MHz): δ 6.60 (bd, 1H, J=1.8 Hz); 4.01-3.95 (m, 1H); 3.71-3.60 (m, 2H); 3.50-3.42 (m, 1H); 3.05-2.85 (m, 2H); 2.39-2.28 (m, 1H); 1.70-1.55 (m, 2H); 0.95 (t, 3H, J=7.5 Hz).

### Example 72

Trifluoroacetamide 207: To a degassed solution of amino acid 206 (59 mg, 0.246 mmol) in dry methanol (1.0 mL) under argon was added Et₃N (35 μL) followed by methyl trifluoroacetate (35 μL). The reaction was stirred for one week at room temperature and concentrated. Analysis by ¹H NMR showed that reaction was 40% complete. The crude reaction product was redissolved in dry methanol (1.0 mL), methyl trifluoroacetate (1.0 mL) and Et₃N (0.5 mL) and stirred at room temperature for 5 days. The reaction was then concentrated in vacuo and dissolved in 50% aqueous THF (2.0 mL), acidified to pH 4 with Amberlite IR-120 (H⁺) acidic resin and filtered. Concentration gave the crude trifluoroacetamide carboxylic acid which was used without further purification for the next reaction.

### Example 73

Amino acid 208: A solution of azide 207 (crude from previous reaction) in THF (2.0 mL) and water (160 μL) was treated with polymer supported triphenyl phosphine (225 mg) at room temperature. After stirring for 20 h the polymer was filtered and washed with methanol. Concentration in vacuo gave a pale solid which was purified by C₁₈ reverse phase chromatography eluting with water. Fractions containing the desired product were pooled and lyophilized to give 6.5 mg (9%) of the trifluoroacetamide amino acid 208. ¹H NMR (D₂O, 300 MHz): δ 6.59 (bs, 1H); 4.40-4.30 (m, 1H); 4.26 (t, 1H, J=10.1 Hz); 3.80-3.66 (m, 2H); 3.56-3.47 (m, 1H); 2.96 (bdd, 1H, J=5.4, 17.7 Hz); 2.58-2.45 (m, 1H); 1.62-1.50 (m, 2H); 0.89 (t, 3H, J=7.5 Hz).

### Example 74

Methylsulfonamide methyl ester 209: Methanesulfonyl chloride (19 μL) was added to a solution of amine 205 (58 mg, 0.23 mmol, prepared by Example 107), Et₃N (97 μL) and a catalytic amount of DMAP (few crystals) in CH₂Cl₂ (1.0 mL) at 0° C. After 30 min the reaction mixture was warmed to room temperature and stirred for an additional 1 h. Concentration in vacuo followed by flash chromatography of the residue on silica gel (50% hexanes in ethyl acetate) gave 61 mg (79%) of the sulfonamide 209. ¹H NMR (CDCl₃, 300 MHz): δ 6.87 (t, 1H, J=2.3 Hz); 5.08 (d, 1H, J=7.5 Hz); 4.03-3.90 (m, 1H); 3.78 (s, 3H); 3.75-3.45 (m, 4H); 3.14 (s, 3H); 2.95 (dd, 1H, J=5.2, 17.3 Hz); 2.42-2.30 (m, 1H); 1.75-1.55 (m, 2H); 0.95 (t, 3H, J=7.5Hz).

### Example 75

Amino ester 210: A solution of azide 209 (61 mg, 0.183 mmol) in THF (2.0 mL) and water (118 μL) was treated with polymer supported triphenyl phosphine (170 mg) at room temperature. After stirring for 17.5 h the polymer was filtered and washed with methanol. Concentration in vacuo followed by flash chromatography of the residue through a short silica gel column (100% methanol) gave 45 mg (80%) of the amino ester 210 as a pale foam. ¹H NMR (CDCl₃, 300 MHz): δ 6.85 (s, 1H); 3.94 (bd, 1H, J=7.8 Hz); 3.77 (s, 3H); 3.74-3.60 (m, 2H); 3.55-3.45 (m, 1H); 3.25-3.15 (m, 1H); 3.11 (s, 3H); 2.94-2.85 (m, 1H); 2.85 (bs, 2H); 2.22-2.10 (m, 1H); 1.70-1.56 (m, 2H); 0.94 (t, 3H, J=7.5 Hz).

### Example 76

Amino acid 211: A solution of methyl ester 210 (21 mg, 0.069 mmol) in THF (200 μL) was treated with aqueous KOH (135 μL of a 1.039 N solution). The reaction mixture was stirred at room temperature for 40 min and neutralized to pH 7.0 with Amberlite IR-120 (H⁺) acidic resin. The resin was filtered and washed with water and methanol. Concentration in vacuo gave the amino acid as a pale solid which was purified by C₁₈ reverse phase chromatography eluting with water. Fractions containing the desired product were pooled and lyophilized to give 3.5 mg (17%) of the amino acid 211. ¹H NMR (D₂O, 300 MHz): δ 6.60 (d, 1H, J=1.8 Hz); 4.30-4.20 (m, 1H); 3.84-3.75 (m, 1H); 3.68-3.58 (m, 1H); 3.60-3.40 (m, 2H); 3.20 (s, 3H); 2.96-2.88 (m, 1H); 2.55-2.45 (m, 1H); 1.72-1.59 (m, 2H); 0.93 (t, 3H, J=7.4 Hz).

### Example 77

Bis-Boc guanidino ester 212: Treated according to the procedure of Kim and Qian, “Tetrahedron Lett.” 34:7677 (1993). To a solution of amine 210 (31 mg, 0.101 mmol), bis-Boc thiourea (28.5 mg, 0.103 mmol) and Et₃N (47 μL) in dry DMF (203 μL) cooled to 0° C. was added HgCl₂ (30 mg, 0.11 mmol) in one portion. The heterogeneous reaction mixture was stirred for 30 min at 0° C. and then at room temperature for 30 min, after which the reaction was diluted with EtOAc and filtered through a pad of celite. Concentration in vacuo followed by flash chromatography of the residue on silica gel (40% hexanes in ethyl acetate) gave 49 mg (89%) of 212 as a pale oil. ¹H NMR (CDCl₃, 300 MHz): δ 11.47 (s, 1H); 8.66 (d, 1H, J=8.4 Hz); 6.87 (s, 1H); 6.01 (bs, 1H); 4.50-4.35 (m, 1H); 4.04 (bd, 1H, J=8.4 Hz); 3.76 (s, 3H); 3.70-3.60 (m, 1H); 3.53-3.45 (m, 2H); 3.02 (s, 3H); 2.85 (dd, 1H, J=5.3, 17.3 Hz); 2.42-2.30 (m, 1H); 1.66-1.55 (m, 2H); 1.49 (s, 9H); 1.48 (s, 9H); 0.93 (t, 3H, J=7.3 Hz).

### Example 78

Carboxylic acid 213: To a solution of methyl ester 212 (49 mg, 0.090 mmol) in THF (1.0 mL) was added aqueous KOH (260 μL of a 1.039 N solution). The reaction mixture was stirred at room temperature for 16 h, cooled to 0° C. and acidified to pH 4.0 with Amberlite IR-120 (H⁺) acidic resin. The resin was filtered and washed with water and methanol. Concentration in vacuo gave the free acid as a pale foam which was used without further purification in the next reaction.

### Example 79

Guanidine carboxylic acid 214: To a solution of bis-Boc guanidnyl acid 213 (crude from previous reaction) in CH₂Cl₂ (2.0 mL) cooled to 0° C. was added neat trifluoroacetic acid (2.0 mL). The reaction mixture was stirred at 0° C. for 1 h and then at room temperature for 1 h. Concentration in vacuo gave a pale orange solid which was purified by C₁₈ reverse phase chromatography eluting with water. Fractions containing the desired product were pooled and lyophilized to give 10 mg (25%, 2 steps) of the guanidine carboxylic acid 214. ¹H NMR (D₂O, 300 MHz): δ 6.60 (bs, 1H); 4.22 (bd, 1H, J=9.0 Hz); 3.82-3.66 (m, 2H); 3.65-3.54 (m, 1H); 3.43 (bt, 1H, J=9.9 Hz); 3.15 (s, 3H); 2.82 (dd, 1H); J=5.0, 17.5 Hz); 2.48-2.30 (m, 1H); 1.71-1.58 (m, 2H); 0.93 (t, 3H, J=7.3 Hz).

### Example 80

Propionamide methyl ester 215: Propionyl chloride (96 μL, 1.1 mmol) was added to a solution of amine 205 (178 mg, 0.70 mmol, prepared by Example 107) and pyridine (1.5 mL) in CH₂Cl₂ (2.0 mL) cooled to 0° C. After 30 min at 0° C. the reaction was concentrated and partitioned between ethyl acetate and brine. The organic layer was separated and washed sequentially with saturated sodium bicarbonate, brine and dried over MgSO₄. Concentration in vacuo followed by flash chromatography of the residue on silica gel (40% hexanes in ethyl acetate) gave 186 mg (86%) of the propionamide methyl ester 215 as a pale yellow solid. ¹H NMR (CDCl₃, 300 MHz): δ 6.86 (t, 1H, J=2.3 Hz); 5.72 (bd, 1H, J=7.8 Hz); 4.52-4.49 (m, 1H); 4.25-4.15 (m, 1H); 3.77 (s, 3H); 3.65-3.37 (complex m, 3H); 2.87 (dd, 1H, J=5.7, 17.7 Hz); 2.28 (q, 2H, J=7.5 Hz); 2.25-2.20 (m, 1H); 1.65-1.50 (m, 2H); 1.19 (t, 3H, J=7.5 Hz); 0.92 (t, 3H); J=7.5 Hz).

### Example 81

Amino methyl ester 216: A solution of azide 215 (186 mg, 0.60 mmol) in THF (5.0 mL) and water (400 μL) was treated with polymer supported triphenyl phosphine (560 mg) at room temperature. After stirring for 21 h the polymer was filtered and washed with methanol. Concentration in vacuo gave the crude amino ester 216 which was used without any further purification for the next step.

### Example 82

Amino acid 217: A solution of methyl ester 216 (crude from previous reaction) in THF (500 μL) was treated with aqueous KOH (866 μL of a 1.039 N solution). The reaction mixture was stirred at room temperature for 3 h and neutralized to pH 7.0 with Amberlite IR-120 (H⁺) acidic resin. The resin was filtered and washed with water and methanol. Concentration in vacuo gave the amino acid as a pale solid which was purified by C₁₈ reverse phase chromatography eluting with water. Fractions containing the desired product were pooled and lyophilized to give 49 mg (31% 2 steps) of the amino acid 217. ¹H NMR (D₂O, 300 MHz): δ 6.54 (s, 1H); 4.25 (bd, 1H, J=8.7 Hz); 4.13 (dd, 1H, J=9.0, 11.3 Hz); 3.74-3.60 (m, 1H); 3.61-3.40 (m, 2H); 2.85 (dd, 1H, J=5.9, 17.1 Hz); 2.55-2.40 (m, 1H); 2.35 (q, 2H, J=7.5 Hz); 1.65-1.45 (m, 2H); 1.13 (t, 3H); J=7.5 Hz); 0.88 (t, 3H, J=7.5 Hz).

### Example 83

(mono methyl) bis-Boc guanidino ester 218: To a solution of amine 200 (51 mg, 0.19 mmol) and mono methyl bis-Boc thiourea (36 mg, 0.19 mmol) in dry DMF (1.0 mL), was added 1-(3-Dimethylaminopropyl)-3-ethylcarbodiimide hydrochloride (38 mg) and Et₃N (56 μL) at room temperature. After 1.5 h at room temperature HgCl₂ (75 mg, excess) was added in one portion. The heterogeneous reaction mixture was stirred for 45 min, diluted with ethyl acetate and filtered through a pad of celite. The filtrate was diluted with additional ethyl acetate and washed with dilute HCl, saturated sodium bicarbonate, brine and dried over MgSO₄. Concentration in vacuo followed by flash chromatography of the residue on silica gel (10% methanol in ethyl acetate) gave 13 mg (16%) of the (mono methyl) bis-Boc guanidino ester 218 as a colorless foam. ¹H NMR (CDCl₃, 300 MHz): δ 6.84 (s, 1H); 6.20 (bd, 1H, J=5.1 Hz); 5.45 (bs, 1H); 4.25-4.40 (bm, 1H); 4.20-4.05 (bm, 2H); 3.76 (s, 3H); 3.60-3.50 (m, 1H); 3.43-3.30 (m, 1H); 2.90 (dd, 1H, J=5.4, 17.7 Hz); 2.77 (d, 3H, J=4.8 Hz); 2.35-2.25 (m, 1H); 1.96 (s, 3H); 1.60-1.50 (m, 2H); 1.47 (s, 9H); 0.91 (t, 3H, J=7.2 Hz).

### Example 84

(mono methyl) bis-Boc guanidino acid 219: To a solution of methyl ester 218 (13 mg, 0.031 mmol) in THF (500 μL) was added aqueous KOH (60 μL of a 1.039 N solution). The reaction mixture was stirred at room temperature for 1 h and then gently refluxed for 1 h. The reaction was cooled to 0° C. and acidified to pH 6.0 with Amberlite IR-120 (H⁺) acidic resin. The resin was filtered and washed with water and methanol. Concentration in vacuo gave the free acid 219 which was used without further purification in the next reaction.

### Example 85

(mono methyl) guanidino amino acid 220: To a solution of (mono methyl) bis-Boc guanidnyl acid 219 (crude from previous reaction) in CH₂Cl₂ (1.0 mL) cooled to 0° C. was added neat trifluoroacetic acid (1.0 mL). The reaction mixture was stirred at 0° C. for 1 h and then at room temperature for 1 h. Concentration in vacuo gave a pale solid which was purified by C₁₈ reverse phase chromatography eluting with water. Fractions containing the desired product were pooled and lyophilized to give 4.4 mg (33%, 2 steps) of the guanidine carboxylic acid 220. ¹H NMR (D₂O, 300 MHz): δ 6.52 (bs, 1H); 4.27 (bd, 1H, J=8.4 Hz); 4.01 (dd, 1H, J=9.2, 10.3 Hz); 3.86-3.75 (m, 1H); 3.75-3.67 (m, 1H); 3.60-3.49 (m, 1H); 2.85 (s, 3H); 2.80 (dd, 1H, J=5.1, 17.7 Hz); 2.47-2.37 (m, 1H); 2.04 (s, 3H); 1.64-1.50 (m, 2H); 0.90 (t, 3H, J=7.2 Hz).

### Example 86

(R)-methyl propyl ester 221: BF₃.Et₂O (63 μL, 0.51 mmol) was added to a solution of N-trityl aziridine 183 (150 mg, 0.341 mmol) in (R)-(−)-2-butanol (1.2 mL) under argon with stirring at room temperature. The pale solution was heated at 70° C. for 2 h and then concentrated in vacuo to give a brown residue which was dissolved in dry pyridine (2.0 mL) and treated with acetic anhydride (225 μL) and a catalytic amount of DMAP (few crystals) at 0° C. The reaction was allowed to warm to room temperature and stirred for 2 h, concentrated in vacuo and partitioned between ethyl acetate and brine. The organic layer was separated and washed sequentially with dilute HCl, saturated sodium bicarbonate, brine and dried over MgSO₄. Concentration in vacuo followed by flash chromatography of the residue on silica gel (50% hexanes in ethyl acetate) gave 75 mg (72%) of the (R)-methyl propyl ester 221 as a pale solid. ¹H NMR (CDCl₃, 300 MHz): δ 6.79 (t, 1H, J=2.2 Hz); 6.14 (d, 1H, J=7.3 Hz); 4.55 (bd, 1H, J=8.7 Hz); 4.33-4.23 (m, 1H); 3.77 (s, 3H); 3.56-3.45 (m, 1H); 3.40-3.27 (m, 1H); 2.85 (dd, 1H, J=5.5, 17.5 Hz); 2.30-2.15 (m, 1H); 2.04 (s, 3H); 1.5901.40 (m, 2H); 1.10 (d, 3H, J=6.0 Hz); 0.91 (t, 3H, J=7.4 Hz).

### Example 87

(R)-methyl propyl amino ester 222: Ph₃P (95 mg, 0.36 mmol) was added in one portion to a solution of azide 221 (75 mg, 0.24 mmol) and water (432 μL) in THF (3.0 mL). The pale yellow solution was then heated at 50° C. for 10 h, cooled and concentrated in vacuo to give a pale solid. Purification by flash chromatography on silica gel (50% methanol in ethyl acetate) gave 66 mg (97%) of the amino ester 222 as a pale solid.

### Example 88

Amino acid 223: A solution of methyl ester 222 (34 mg, 0.12 mmol) in THF (1.0 mL) was treated with aqueous KOH (175 μL of a 1.039 N solution). The reaction mixture was stirred at room temperature for 3 h and acidified to pH 6.0 with Amberlite IR-120 (H⁺) acidic resin. The resin was filtered and washed with water and methanol. Concentration in vacuo gave the amino acid as a pale solid which was purified by C₁₈ reverse phase chromatography eluting with water. Fractions containing the desired product were pooled and lyophilized to give 11.5 mg (36%) of the amino acid 223. ¹H NMR (D₂O, 300 MHz): δ 6.52 (bs, 1H); 4.28 (bd, 1H, J=8.7 Hz); 4.04 (dd, 1H, J=8.8, 11.5 Hz); 3.74-3.65 (m, 1H); 3.50-3.60 (m, 1H); 2.90 (dd, 1H, J=5.5, 17.2 Hz); 2.50-2.40 (m, 1H0; 2.10 (s, 3H); 1.60-1.45 (m, 2H); 1.14 (d, 3H, J=6.2 Hz); 0.91 (t, 3H); J=7.4 Hz).

### Example 89

bis-Boc guanidino ester 224: Treated according to the procedure of Kim and Qian, “Tetrahedron Lett.”, 34:7677 (1993). To a solution of amine 222 (32 mg, 0.113 mmol), bis-Boc thiourea (32 mg, 0.115 mmol) and Et₃N (53 μL) in dry DMF (350 μL) cooled to 0° C. was added HgCl₂ (34 mg, 0.125 mmol) in one portion. The heterogeneous reaction mixture was stirred for 45 min at 0° C. and then at room temperature for 1 h, after which the reaction was diluted with EtOAc and filtered through a pad of celite. Concentration in vacuo followed by flash chromatography of the residue on silica gel (20% hexanes in ethyl acetate) gave 57 mg (96%) of 224 as a colorless foam. ¹H NMR (CDCl₃, 300 MHz): δ 11.40 (s, 1H); 8.65 (d, 1H, J=7.8 Hz); 6.82 (s, 1H); 6.36 (d, 1H, J=8.7 Hz); 4.46-4.34 (m, 1H); 4.20-4.10 (m, 1H); 4.10-3.95 (m, 1H); 3.76 (s, 3H); 2.79 (dd, 1H, J=5.4, 17.7 Hz); 2.47-2.35 (m, 1H); 1.93 (s, 3H); 1.60-1.45 (m, 2H); 1.49 (s, 18H); 1.13 (d, 3H, J=6.0 Hz); 0.91 (t, 3H, J=7.5 Hz).

### Example 90

Carboxylic acid 225: To a solution of methyl ester 224 (57 mg, 0.11 mmol) in THF (1.5 mL) was added aqueous KOH (212 μL of a 1.039 N solution). The reaction mixture was stirred at room temperature for 16 h, cooled to 0° C. and acidified to pH 4.0 with Amberlite IR-120 (H⁺) acidic resin. The resin was filtered and washed with water and methanol. Concentration in vacuo gave the free acid as a pale foam which was used without further purification in the next reaction.

### Example 91

Guanidine carboxylic acid 226: To a solution of bis-Boc guanidnyl acid 225 (crude from previous reaction) in CH₂Cl₂ (4.0 mL) cooled to 0° C. was added neat trifluoroacetic acid (4.0 mL). The reaction mixture was stirred at 0° C. for 1 h and then at room temperature for 2 h. Concentration in vacuo gave a pale orange solid which was purified by C₁₈ reverse phase chromatography eluting with water. Fractions containing the desired product were pooled and lyophilized to give 18.4 mg (40%, 2 steps) of the guanidine carboxylic acid 226. ¹H NMR (D₂O, 300 MHz): δ 6.47 (s, 1H); 4.28 (bd, 1H, J=8.4 Hz); 3.93-3.74 (m, 2H); 3.72-3.63 (m, 1H); 2.78 (dd, 1H, J=4.8, 17.4 Hz); 2.43-2.32 (m, 1H); 1.58-1.45 (m, 2H); 1.13 (d, 3H, J=6.0 Hz); 0.90 (t, 3H, J=7.4 Hz).

### Example 92

(Diethyl) methyl ether ester 227: BF₃.Et₂O (6.27 mL, 51 mmol) was added to a solution of N-trityl aziridine 183 (15 g, 34 mmol) in 3-pentanol (230 mL) under argon with stirring at room temperature. The pale solution was heated at 70-75° C. for 1.75 h and then concentrated in vacuo to give a brown residue which was dissolved in dry pyridine (2.0 mL) and treated with acetic anhydride (16 mL, 170 mmol) and a catalytic amount of DMAP 200 mg. The reaction was stirred at room temperature for 18 h, concentrated in vacuo and partitioned between ethyl acetate and 1M HCl. The organic layer was separated and washed sequentially with saturated sodium bicarbonate, brine and dried over MgSO₄. Concentration in vacuo followed by flash chromatography of the residue on silica gel (50% hexanes in ethyl acetate) gave 7.66 g of the (Diethyl) methyl ether ester which was recrystallized from ethylacetate/hexane to afford 227 (7.25 g, 66%) as colorless needles: ¹H NMR (CDCl₃, 300 MHz): δ 6.79 (t, 1H, J=2.1 Hz); 5.92 (d, 1H, J=7.5 Hz); 4.58 (bd, 1H, J=8.7 Hz); 4.35-4.25 (m, 1H); 3.77 (s, 3H); 3.36-3.25 (m, 2H); 2.85 (dd, 1H), J=5.7, 17.4 Hz); 2.29-2.18 (m, 1H); 2.04 (s, 3H); 1.60-1.45 (m, 4H); 0.91 (t, 3H, J=3.7 Hz); 0.90 (t, 3H, J=7.3 Hz).

### Example 93

(Diethyl) methyl ether amino ester 228: Ph₃P (1.21 g, 4.6 mmol) was added in one portion to a solution of azide 227 (1 g, 3.1 mmol) and water (5.6 mL) in THF (30 mL). The pale yellow solution was then heated at 50° C. for 10 h, cooled and concentrated in vacuo. The aqueous oily residue was partitioned between EtOAc and saturated NaCl. The organic phase was dried (MgSO₄), filtered, and evaporated. Purification by flash chromatography on silica gel (50% methanol in ethyl acetate) gave 830 mg (90%) of the amino ester 228 as a pale white solid. ¹H NMR (CDCl₃, 300 MHz): δ 6.78 (t, 1H, J=2.1 Hz); 5.68 (bd, 1H, J=7.8 Hz); 4.21-4.18 (m, 1H); 3.75 (s, 3H); 3.54-3.45 (m, 1H); 3.37-3.15 (m, 2H); 2.74 (dd, 1H, J=5.1, 17.7 Hz); 2.20-2.07 (m, 1H); 2.03 (s, 3H); 1.69 (bs, 2H, —NH₂); 1.57-1.44 (m, 4H); 0.90 (t, 3H, J=7.5 Hz); 0.89 (t, 3H, J=7.5 Hz).

### Example 94

Amino acid 229: A solution of methyl ester 228 (830 mg, 2.8 mmol) in THF (15 mL) was treated with aqueous KOH (4 mL of a 1.039 N solution). The reaction mixture was stirred at room temperature for 40 min and acidified to pH 5.5-6.0 with Dowex 50WX8 acidic resin. The resin was filtered and washed with water and methanol. Concentration in vacuo gave the amino acid as a pale solid which was purified by C₁₈ reverse phase chromatography eluting with water and then with 5% CH₃CN/water. Fractions containing the desired product were pooled and lyophilized to give 600 mg (75%) of the amino acid 229. ¹H NMR (D₂O, 300 MHz): δ 6.50 (t, 1H, J=2.1 Hz); 4.30-4.26 (m, 1H); 4.03 (dd, 1H, J=9.0, 11.7 Hz); 3.58-3.48 (m, 2H); 2.88 (dd, 1H, J=5.4, 16.8 Hz); 2.53-2.41 (m, 1H); 1.62-1.40 (m, 4H); 0.90 (t, 3H, J=7.5 Hz); 0.85 (t, 3H, J=7.5 Hz).

### Example 95

t-amyl ether ester 230: BF₃.Et₂O (43 μL, 0.35 mmol) was added to a solution of N-trityl aziridine 183 (104 mg, 0.24 mmol) in t-amyl alcohol (2.5 mL) under argon with stirring at room temperature. The pale solution was heated at 75° C. for 3 h and then concentrated in vacuo to give a brown residue which was dissolved in dry pyridine (2.0 mL) and treated with acetic anhydride (250 μL) and a catalytic amount of DMAP (few crystals). The reaction was stirred at room temperature for 1.5 h, concentrated in vacuo and partitioned between ethyl acetate and brine. The organic layer was separated and washed sequentially with dilute HCl, saturated sodium bicarbonate, brine and dried over MgSO₄. Concentration in vacuo followed by flash chromatography of the residue on silica gel (50% hexanes in ethyl acetate) gave 27 mg (35%) of the t-amyl ether ester 230 as a pale orange oil. ¹H NMR (CDCl₃, 300 MHz): δ 6.72 (t, 1H, J=2.1 Hz); 5.83 (d, 1H, J=7.2 Hz); 4.71 (bd, 1H, J=8.1 Hz); 4.45-4.35 (m, 1H); 3.75 (s, 3H); 3.27-3.17 (m, 1H); 2.84 (dd, 1H, J=5.7, 17.4 Hz); 2.27-2.15 (m, 1H); 2.05 (s, 3H); 1.57-1.47 (m, 2H); 1.19 (s, 3H); 1.15 (s, 3H); 0.90 (t, 3H, J=7.5 Hz).

### Example 96

t-amyl ether amino ester 231: Ph₃P (35 mg, 0.133 mmol) was added in one portion to a solution of azide 230 (27 mg, 0.083 mmol) and water (160 μL) in THF (1.5 mL). The pale orange solution was then heated at 50° C. for 10 h, cooled and concentrated in vacuo to give a pale solid. Purification by flash chromatography on silica gel (50% methanol in ethyl acetate) gave 20 mg (82%) of the amino ester 231 as a pale oil.

### Example 97

Amino acid 232: A solution of methyl ester 231 (20 mg, 0.068 mmol) in THF (1.0 mL) was treated with aqueous KOH (131 μL of a 1.039 N solution). The reaction mixture was stirred at room temperature for 2.5 h and acidified to pH 5.0 with Amberlite IR-120 (H⁺) acidic resin. The resin was filtered and washed with water and methanol. Concentration in vacuo gave the amino acid as a pale solid which was purified by C₁₈ reverse phase chromatography eluting with water. Fractions containing the desired product were pooled and lyophilized to give 8.6 mg (45%) of the amino acid 232. ¹H NMR (D₂O, 300 MHz): δ 6.47 (bs, 1H); 4.42 (bd, 1H, J=8.1 Hz); 3.97 (dd, 1H, J=8.4, 11.4 Hz); 3.65-3.54 (m, 1H); 2.88 (dd, 1H, J=5.5, 17.3 Hz); 2.51-2.39 (m, 1H); 2.08 (s, 3H); 1.61-1.46 (m, 2H); 1.23 (s, 3H); 1.18 (s, 3H), 0.86 (t, 3H, J=7.5 Hz).

### Example 98

n-Propyl thio ether ester 233: BF₃.Et₂O (130 μL, 1.06 mmol) was added to a solution of N-trityl aziridine 183 (300 mg, 0.68 mmol) in 1-propanethiol (8.0 mL) under argon with stirring at room temperature. The pale solution was then heated at 65° C. for 45 min, concentrated and partitioned between ethyl acetate and brine. The organic layer was separated and washed with saturated sodium bicarbonate, brine and dried over MgSO₄. Concentration in vacuo followed by flash chromatography of the residue on silica gel (30% hexanes in ethyl acetate) gave 134 mg (73%) of the n-propyl thio ether ester 233 as a pale oil. ¹H NMR (CDCl₃, 300 MHz): δ 6.87 (t, 1H, J=2.4 Hz); 3.77 (s, 3H); 3.48-3.38 (m, 1H); 3.22-3.18 (m, 1H), 2.93 (dd, 1H, J=5.4, 17.4 Hz); 2.80 (t, 1H, J=9.9 Hz); 2.51 (t, 2H, J=7.2 Hz); 2.32-2.20 (m, 1H); 1.96 (bs, 2H, —NH₂), 1.69-1.56 (m, 2H); 1.00 (t, 3H, J=7.2 Hz).

### Example 99

n-Propyl thio ether azido ester 234: To a solution of amine 233 (134 mg, 0.50 mmol) in pyridine (1.5 mL) cooled to 0° C. was added neat acetyl chloride (60 μL, 0.84 mmol). After stirring for 1 h the reaction mixture was warmed to room temperature and stirred for an additional 15 min. The reaction was concentrated and partitioned between ethyl acetate and brine and washed sequentially with dilute HCl, water, saturated sodium bicarbonate, brine and dried over MgSO₄. Concentration in vacuo followed by flash chromatography of the residue on silica gel (30% hexanes in ethyl acetate) gave 162 mg (100%) of the n-Propyl thio ether azido ester 234 as a pale yellow solid. ¹H NMR (CDCl₃, 300 MHz): δ 6.90 (t, 1H, J=2.7 Hz); 5.87 (bd, 1H, J=7.8 Hz); 4.07-3.98 (m, 1H); 3.77 (s, 3H); 3.65-3.55 (m, 1H); 2.95-2.85 (m, 1H); 2.60-2.45 (m, 2H); 2.30-218 (m, 1H); 2.08 (s, 3H); 1.65-1.53 (m, 2H); 0.98 (t, 3H, J=7.2 Hz).

### Example 100

n-Propyl thio ether amino ester 235: The azide 234 (130 mg, 0.416 mmol) in ethyl acetate (10 mL) was hydrogenated (1 atmosphere) over Lindlar's catalyst (150 mg) for 18 h at room temperature. The catalyst was then filtered through a celite pad and washed with hot ethyl acetate and methanol. Concentration in vacuo followed by flash chromatography of the orange residue gave 62 mg (53%) of the n-propyl thio ether amino ester 235. ¹H NMR (CDCl₃, 300 MHz): δ 6.88 (t, 1H, J=2.7 Hz); 5.67 (bd, 1H, J=8.7 Hz); 3.76 (s, 3H); 3.75-3.65 (m, 1H); 3.45-3.35 (bm, 1H); 3.05-2.95 (m, 1H); 2.87-2.78 (m, 1H); 2.56-2.40 (m, 2H); 2.18-2.05 (m, 1H); 2.09 (s, 3H); 1.65-1.50 (m, 2H); 1.53 (bs, 2H, —NH₂); 0.98 (t, 3H, J=7.2 Hz).

### Example 101

Compound 240: A suspension of Quinic acid (103 g), 2,2-dimethoxypropane (200 mL) and toluenesulfonic acid (850 mg) in acetone (700 mL) was stirred at room temperature for 4 days. Solvents and excess reagents were removed under reduced pressure. Purification by flash column chromatography (Hexanes/EtOAc=2/1-1.5/1) gave lactone 240 (84 g, 73%): ¹H NMR (CDCl₃) δ 4.72 (dd, J=2.4, 6.1 Hz, 1H), 4.50 (m, 1H), 4.31 (m, 1H), 2.67 (m, 2H), 2.4-2.2 (m, 3H), 1.52 (s, 3H), 1.33 (s, 3H). Performing the reaction at reflux temperatures for 4 h afforded lactone 240 in 71% yield after aqueous work-up (ethyl acetate/water partition) and recrystallization of the crude product from ethyl acetate/hexane.

### Example 102

Compound 241: To a solution of lactone 240 (43.5 g, 203 mmol) in methanol (1200 mL) was added sodium methoxide (4.37 M, 46.5 ml, 203 mmol) in one portion. The mixture was stirred at room temperature for 3 hrs, and quenched with acetic acid (11.62 mL). Methanol was removed under reduced pressure. The mixture was diluted with water, and extracted with EtOAc (3×). The combined organic phase was washed with water (1×) and brine (1×), and dried over MgSO₄. Purification by flash column chromtography (Hexanes/EtOAc=1/1 to 1/4) gave diol (43.4 g, 87%): ¹H NMR (CDCl₃) δ 4.48 (m, 1H), 4.13 (m, 1H), 3.99 (t, J=6.4 Hz, 1H), 3.82 (s, 3H), 3.34 (s, 1H), 2.26 (d, J=3.8 Hz, 2H), 2.08 (m, 1H), 1.91 (m, 1H), 1.54 (s, 3H), 1.38 (s, 3H). Alternatively, treatment of lactone 240 with catalytic sodium ethoxide (1 mol %) in ethanol gave the corresponding ethyl ester in 67% after crystallization of the crude product from ethyl acetate/hexane. The residue obtained from the mother liquor (consisting of starting material and product) was subjected again to the same reaction conditions, affording additional product after recrystallization. Overall yield was 83%.

### Example 103

Compound 242: To a solution of diol 241 (29.8 g, 121 mmol) and 4-(N,N-dimethylamino)pyridine (500 mg) in pyridine (230 mL) was added tosyl chloride (27.7 g, 145 mmol). The mixture was stirred at room temperature for 3 days, and pyridine was removed under reduced pressure. The mixture was diluted with water, and extracted with EtOAc (3×). The combined organic phase was washed with water (2×) and brine (1×), and dried over MgSO₄. Concentration and purification by flash column chromatography (Hexanes/EtOAc=2/1-1/1) gave tosylate 242 (44.6 g, 92%): ¹H NMR (CDCl₃) δ 7.84 (d, J=8.4 Hz, 2H), 7.33 (d, J=8.1 Hz, 2H), 4.76 (m, 1H), 4.42 (m, 1H), 4.05 (dd, J=5.5, 7.5 Hz, 1H), 3.80 (s, 3H), 2.44 (s, 3H), 2.35 (m, 1H), 2.24 (m, 2H), 1.96 (m, 1H), 1.26 (s, 3H), 1.13 (s, 3H). The corresponding ethyl ester of compound 241 was treated with methanesulfonyl chloride and triethylamine in CH₂Cl₂ at 0° C. to afford the mesylate derivative in quantitative yield after aqueous work-up. The mesylate was used directly without any further purification.

### Example 104

Compound 243: To a solution of tosylate 242 (44.6 g, 111.5 mmol) in CH₂Cl₂ (450 mL) at −78° C. was added pyridine (89 mL), followed by slow addition of SO₂Cl₂ (26.7 mL, 335 mmol). The mixture was stirred at −78° C. for 5 hrs, and methanol (45 mL) was added dropwise. The mixture was warmed to room temperature and stirred for 12 hrs. Ethyl ether was added, and the mixture was washed with water (3×) and brine (1×), and dried over MgSO₄. Concentration gave the intermediate as a oil (44.8 g). To a solution of the intermediate (44.8 g, 111.5 mmol) in MeOH (500 mL) was added TsOH (1.06 g, 5.6 mmol). The mixture was refluxed for 4 hrs. The reaction mixture was cooled to room temperature, and methanol was removed under reduced pressure. Fresh methanol (500 mL) was added, and the whole mixture was refluxed for another 4 hrs. The reaction mixture was cooled to room temperature, and methanol was removed under reduced pressure. Purification by flash column chromatography (Hexanes/EtOAc=3/1-1/3) gave a mixture of the two isomers (26.8 g). Recrystalization from EtOAc/Hexanes afforded the pure desired product 243 (20.5 g, 54%): ¹H NMR (CDCl₃) δ 7.82 (d, J=8.3 Hz, 2H), 7.37 (d, J=8.3 Hz, 2H), 6.84 (m, 1H), 4.82 (dd, J=5.8, 7.4 Hz, 1H), 4.50 (m, 1H), 3.90 (dd, J=4.4, 8.2 Hz, 1H), 3.74 (s, 3H), 2.79 (dd, J=5.5, 18.2 Hz, 1H), 2.42 (dd, J=6.6, 18.2 Hz, 1H). The corresponding mesylate-ethyl ester derivative of compound 242 was treated in the same manner as described. Removal of the acetonide protecting group was accomplished with acetic acid in refluxing ethanol to afford the diol in 39% yield by direct precipitation with ether from the crude reaction mixture.

### Example 105

Compound 1: To a solution of diol 243 (20.0 g, 58.5 mmol) in THF (300 mL) at 0° C. was added DBU (8.75 mL, 58.5 mmol). The reaction mixture was warmed to room temperature, and stirred for 12 hrs. Solvent (THF) was removed under reduced pressure. Purification by flash column chromatography (Hexanes/EtOAc=1/3) gave epoxide 1 (9.72 g, 100%): ¹H NMR (CDCl₃) δ 6.72 (m, 1H), 4.56 (td, J=2.6, 10.7 Hz, 1H), 3.76 (s, 3H), 3.56 (m, 2H), 3.0 (d, J=21 Hz, 1H), 2.50 (d, J=20 Hz, 1H), 2.11 (d, 10.9 Hz, 1H). The corresponding mesylate-ethyl ester derivative of compound 243 was treated in the same manner as described, affording the epoxide in nearly quantitative yield.

### Example 106

Aziridine 244: A solution of allyl ether 4 (223 mg, 1.07 mmol) and Lindlar's catalyst (200 mg) in absolute ethanol (8.0 mL) was treated with hydrogen gas (1 atmosphere) at room temperature for 50 min. The catalyst was then filtered through a celite pad and washed with hot methanol. Concentration in vacuo gave ˜230 mg of 244 as pale yellow oil which was used for the next reaction without any further purification.

### Example 107

Azido amine 205: Crude aziridine 244 (230 mg), sodium azide (309 mg, 4.75 mmol) and ammonium chloride (105 mg, 1.96 mmol) in dry DMF (10 mL) was heated at 70° C. for 16 h under an argon atmosphere. The reaction was cooled, filtered through a fritted glass funnel to remove solids and partitioned between ethyl acetate and brine. The organic layer was separated and dried over MgSO₄. Concentration in vacuo followed by flash chromatography of the residue on silica gel (10% hexanes in ethyl acetate) gave 154 mg (57%, 2 steps) of 205 as a yellow viscous oil of sufficient purity for the next reaction.

### Example 108

N-acetyl azide 245: Acetyl chloride (70 μl, 0.98 mmol) was added to a solution of amine 205 (154 mg, 0.61 mmol) and pyridine (1.3 mL) in CH₂Cl₂ (4.0 mL) cooled to 0° C. After 1.5 h at 0° C. the reaction was concentrated and partitioned between ethyl acetate and brine. The organic layer was separated and washed sequentially with saturated sodium bicarbonate, brine and dried over MgSO₄. Concentration in vacuo followed by flash chromatography of the residue on silica gel (ethyl acetate) gave 167 mg (93%) of 245 as a pale yellow solid.

### Example 109

Amino ester 200: Triphenyl phosphine (1.7 g, 6.48 mmol) was added in several portions to a solution of 245 (1.78 g, 6.01 mmol) in THF (40 mL) and water (1.5 mL). The reaction was then stirred at room temperature for 42.5 h. Volatiles were removed under vaccum and the crude solid absorbed onto silica gel and purified by flash chromatography on silica gel (100% ethyl acetate then 100% methanol) to give 1.24 g (77%) of 200 as a pale solid.

### Example 110

Amino acid 102: To a solution of methyl ester 200 (368 mg, 1.37 mmol) in THF (4.0 mL) cooled to 0° C. was added aqueous NaOH (1.37 mL of a 1.0 N solution). The reaction mixture was stirred at 0° C. for 10 min, room temperature for 1.5 h and then acidified to pH 7.0-7.5 with Amberlite IR-120 (H⁺) acidic resin. The resin was filtered and washed with water and methanol. Concentration in vacuo gave the amino acid as a white solid which was purified by C₁₈ reverse phase chromatography eluting with water. Fractions containing the desired product were pooled and lyophilized to give 290 mg (83%) of amino acid 102.

### Example 111

Amine hydrochloride 250: Amine 228 (15.6 mg, 0.05 mmol) was treated with 0.1 N HCl and was evaporated. The residue was dissolved in water and was filtered through a small column of C-18 reverse phase silica gel. The hydrochloride salt 250 (12 mg) was obtained as a solid after lyophilization: ¹H NMR (D₂O) δ 6.86 (s, 1H), 4.35 (br d, J=9.0), 4.06 (dd, 1H, J=9.0, 11.6), 3.79 (s, 3H), 3.65-3.52 (m, 2H), 2.97 (dd, 1H, J=5.5, 17.2), 2.58-2.47 (m, 1H), 2.08 (s, 3H), 1.61-1.41 (m, 4H), 0.88 (t, 3H, J=7.4), 0.84 (t, 3H, J=7.4).

### Example 112

Bis-Boc-guanidine 251: To a solution of amine 228 (126 mg, 0.42 mmol), N,N′-bis-tert-butoxycarbonylthiourea (127 mg, 0.46 mmol), and triethylamine (123 μL, 0.88 mmol) in DMF (4 mL) at 0° C. was added HgCl₂ (125 mg, 0.46 mmol). The mixture was stirred at 0° C. for 30 min and at room temperature for 1.5 h. The reaction was diluted with ethyl acetate and filtered through celite. The solvent was evaporated and the residue was partitioned between ethyl acetate and water. The organic phase was washed with saturated NaCl, dried (MgSO₄), filtered and the solvent was evaporated. The crude product was purified on silica gel (2/1, 1/1-hexane/ethyl acetate) to afford bis-Boc-guanidine 251 (155 mg, 69%) as a solid: ¹H NMR (CDCl₃) δ 11.40 (s, 1H), 8.66 (d, 1H, J=7.9), 6.8 (s, 1H), 6.22 (d, 1H, J=8.9), 4.43-4.34 (m, 1H), 4.19-4.08 (m, 1H), 4.03 (m, 1H), 3.76 (s, 3H), 3.35 (m, 1H), 2.79 (dd, 1H, J=5.4, 17.7), 2.47-2.36 (m, 1H), 1.92 (s, 3H), 1.50, 1.49 (2s, 18H), 0.89 (m, 6H).

### Example 113

Guanidino-acid 252: To a solution of bis-Boc-guanidine 251 (150 mg, 0.28 mmol) in THF (3 mL) was added 1.039N KOH solution (337 μL) and water (674 μL). The mixture was stirred for 3 h, additional 1.039N KOH solution (67 μL) was added and stirring was continued for 2 h. The reaction was filtered to remove a small amount of dark precipitate. The filtrate was cooled to 0° C. and was acidified with IR 120 ion exchange resin to pH 4.5-5.0. The resin was filtered and washed with methanol. The filtrate was evaporated to a residue which was dissolved in CH₂Cl₂ (3 mL), cooled to 0° C., and was treated with trifluoroacetic acid (3 mL). After stirring 10 min. at 0° C., the reaction was stirred at room temperature for 2.5 h. The solvents were evaporated and the residue was dissolved in water and was chromatographed on a short column (3×1.5 cm) of C-18 reverse phase silica gel eluting initially with water and then 5% acetonitrile/water. Product fractions were combined and evaporated. The residue was dissolved in water and lyophilized to afford guanidino-acid 252 (97 mg, 79%) as a white solid.

### Example 114

Azido acid 260: To a solution of methyl ester 227 (268 mg, 0.83 mmol) in THF (7.0 mL) was added aqueous KOH (1.60 mL of a 1.039 N solution) at room temperature. After stirring for 19 h at room temperature the reaction was acidified to pH 4.0 with Amberlite IR-120 (H⁺) acidic resin. The resin was filtered and washed with water and ethanol. Concentration in vacuo gave the crude azido acid 260 as a pale orange foam which was used for the next reaction without any further purification.

### Example 115

Azido ethyl ester 261: To a solution of carboxylic acid 260 (crude from previous reaction, assume 0.83 mmol), ethyl alcohol (150 μL), and catalytic DMAP in CH₂Cl₂ (6.0 mL) was added DCC (172 mg, 0.83 mmol) in one portion at room temperature. After several minutes a precipitate formed and after an additional 1 h of stirring the reaction was filtered and washed with CH₂Cl₂. Concentration in vacuo afforded a pale solid which was purified by flash chromatography on silica gel (50% hexanes in ethyl acetate) to give 272 mg (96%, small amount of DCU impurity present) of 261 as a white solid. When DCC was replaced by diisopropyl carbodiimide than the yield of 261 was 93% but the chromatographic purification eliminated urea impurities present when DCC was used.

### Example 116

Amino ethyl ester 262: Triphenyl phosphine (342 mg, 1.30 mmol) was added in one portion to a solution of 261 (272 g, 0.80 mmol) in THF (17 mL) and water (1.6 mL). The reaction was then heated at 50° C. for 10 h, cooled and concentrated in vacuo to give a pale white solid. Purification of the crude solid by flash chromatography on silica gel (50% methanol in ethyl acetate) gave 242 mg (96%) of the amino ethyl ester 262 as a pale solid. The amino ethyl ester is dissolved in 3N HCl and lyophilized to give the corresponding water soluble HCl salt form. ¹H NMR (D₂O, 300 MHz): δ 6.84 (s, 1H); 4.36-4.30 (br m, 1H); 4.24 (q, 2H, J=7.2 Hz); 4.05 (dd, 1H, J=9.0, 11.7 Hz); 3.63-3.50 (m, 2H); 2.95 (dd, 1H, J=5.7, 17.1 Hz); 2.57-2.45 (m, 1H); 1.60-1.39 (m, 4H); 1.27 (t, 3H, J=7.2 Hz); 0.89-0.80 (m, 6H).

### Example 117

bis-Boc guanidino ethyl ester 263: Treated according to the procedure of Kim and Qian, “Tetrahedron Lett.” 34:7677 (1993). To a solution of amine 262 (72 mg, 0.23 mmol), bis-Boc thiourea (66 mg, 0.24 mmol) and Et₃N (108 μL) in dry DMF (600 μL) cooled to 0° C. was added HgCl₂ (69 mg, 0.25 mmol) in one portion. The heterogeneous reaction mixture was stirred for 1 h at 0° C. and then at room temperature for 15 min, after which the reaction was diluted with EtOAc and filtered through a pad of celite. Concentration in vacuo followed by flash chromatography of the residue on silica gel (20% hexanes in ethyl acetate) gave 113 mg (89%) of 263 as a colorless foam. ¹H NMR (CDCl₃, 300 MHz): δ 11.41 (s, 1H); 8.65 (d, 1H, J=8.1 Hz); 6.83 (s, 1H); 6.22 (d, 1H, J=9.0 Hz); 4.46-4.34 (m, 1H); 4.21 (q, 2H, J=6.9 Hz); 4.22-4.10 (m, 1H); 4.04-4.00 (m, 1H); 3.36 (quintet, 1H, J=5.7 Hz); 2.78 (dd, 1H, J=5.4, 17.7 Hz); 2.46-2.35 (m, 1H); 1.94 (s, 3H); 1.60-1.40 (m, 4H); 1.49 (s, 9H); 1.50 (s, 9H); 1.30 (t, 3H, J=6.9 Hz); 0.93-0.84 (m, 6H).

### Example 118

Guanidino ethyl ester 264: To a solution of bis-Boc guanidnyl ethyl ester 263 (113 mg, 0.20 mmol) in CH₂Cl₂ (5.0 mL) cooled to 0° C. was added neat trifluoroacetic acid (5.0 mL). The reaction mixture was stirred at 0° C. for 30 min and then at room temperature for 1.5 h. The reaction was then concentrated in vacuo to give a pale orange solid which was purified by C₁₈ reverse phase chromatography eluting with water. Fractions containing the desired product were pooled and lyophilized to give 63 mg (66%) of the guanidine ethyl ester 264 as white solid. ¹H NMR (D₂O, 300 MHz): δ 6.82 (s, 1H); 4.35-4.31 (m, 1H); 4.24 (q, 2H, J=7.1 Hz); 3.95-3.87 (m, 1H); 3.85-3.76 (m, 1H); 3.57-3.49 (m, 1H); 2.87 (dd, 1H, J=5.1, 17.7 Hz); 2.46-2.34 (m, 1H); 2.20 (s, 3H); 1.60-1.38 9M, 4H); 1.28 (t, 3H, J=7.1 Hz); 0.90-0.80 (m, 6H).

### Example 119

Enzyme Inhibition: Using the methods of screening in vitro activity described above, the following activities were observed (+10-100 μm, ++1-10 μm, +++<1.0 μm):

| Compound | IC₅₀ |
|---|---|
| 102/103 (2:1) | +++ |
| 8 | ++ |
| A.17.a.4.i | ++ |
| 114 | ++ |
| A.1.a.4.i | ++ |
| 79 | + |
| 82/75 (1.2:1) | + |
| 94 | +++ |
| A.100.a.11.i | +++ |
| A.101.a.11.i | +++ |
| A.113.a.4.i | +++ |

### Example 120

Compounds A.113.b.4.i and A.113.x.4.i were incubated separately in enzyme assay buffey and tested for activity as described in Example 119. Activity was >100 μm for both. When each compound was separately incubated in rat plasma prior to testing as described in Example 119, activity of both was similar to compound A.113.a.4.i.

### Example 121

Studies were conducted under the supervision of Dr. Robert Sidwell at the Institute for Antiviral Research of Utah State University to determine the comparative anti-influenza A activity of compound 203 (example 69), GG167 and ribavirin in vivo in mice by i.p. or p.o. routes of administration. GG167 and ribavirin are known anti-influenza virus compounds.

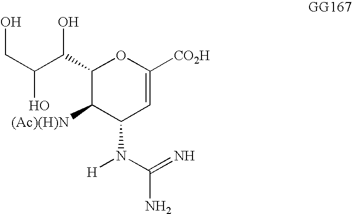

Mice: Female 13-15 g specific-pathogen free BALB/c mice were obtained from Simonsen Laboratories (Gilroy, Calif.). They were quarantined 24 hr prior to use, and maintained on Wayne Lab Blox and tap water. Once infected, the drinking water contained 0.006% oxytetracycline (Pfizer, New York, N.Y.) to control possible secondary bacterial infections.

Virus: Influenza A/NWS/33 (H1N1) was obtained from K. W. Cochran, University of Michigan (Ann Arbor, Mich.). A virus pool was prepared by infecting confluent monolayers of Madin Darby canine kidney (MDCK) cells, incubating them at 37° C. in 5% CO₂, and harvesting the cells at 3 to 5 days when the viral cytopathic effect was 90 to 100%. The virus stock was ampuled and stored at −80° C. until used.

Compounds: Compound 203 and GG167 were dissolved in sterile physiological saline for this study.

Arterial Oxygen Saturation (SaO₂) Determinations: SaO₂ was determined using the Ohmeda Biox 3740 pulse oximeter (Ohmeda, Louisville, Ohio). The ear probe attachment was used, the probe placed on the thigh of the animal, with the slow instrument mode selected. Readings were made after a 30 second stabilization time on each animal. Use of this device for measuring effects of influenza virus on arterial oxygen saturation has been described by Sidwell et al., “Antimicrob. Agents Chemother.” 36:473-476 (1992).

Experiment Design for Intraperitoneal Administration Study: Groups of eleven mice infected intranasally with an approximate 95% lethal dose of virus received each dose of test compound. Doses of both 203 and GG167 were 50, 10, 2 and 0.5 mg/kg/day. Treatments were i.p. twice daily for 5 days beginning 4 hr pre-virus exposure. Eight of the infected, treated mice at each dosage and 16 infected, saline-treated controls were assayed for SaO₂ level on days 3 through 10; deaths were recorded daily in these animals for 21 days. The remaining three animals in each group as well as six saline-treated control mice were killed on day 6 and their lungs removed, weighed, assigned a consolidation score based on extent of plum color in the lungs (0=normal, 4=100% of lung affected). Since no toxicity had been seen at a dose of 300 mg/kg/day of 203 and literature reports indicate GG167 to be similarly nontoxic, toxicity controls were not included in this study.

Experiment Design for Oral Administration Study: Groups of 11 mice were infected intranasally with an approximate 95% lethal dose of virus and treated with 250, 50, or 10 mg/kg/day of 203 or GG167 or with 100, 32 or 10 mg/kg/day of ribavirin. Treatment was by oral gavage (p. o.) twice daily for 5 days beginning 4 hr pre-virus exposure. Eight of the animals in each group were held for 21 days, with deaths noted daily and SaO₂ levels determined on days 3-10. The remaining 3 infected mice in each group were killed on day 6 and their lungs removed, weighed, assigned a consolidation score of 0 (normal) to 4 (100% lung affected). Fifteen infected mice were treated with saline only and held 21 days with SaO₂ determined as above, and 6 additional infected, saline treated mice were killed on day 6 for lung assay. Three normal controls were held 21 days, with SaO₂ determined in parallel with the above, and an additional 3 normal animals were killed on day 6 for lung weight and score.

Experiment Design for Low Dose Oral Administration Study: Groups of 8 mice infected intranasally with an approximate 90% lethal concentration of virus received each dosage of compound. Doses of each compound were 10, 1, and 0.1 mg/kg/day. Treatments were p.o. twice daily for 5 days beginning 4 hr pre-virus exposure. Eight of the infected, treated mice at each dosage and 16 infected, saline-treated controls were assayed for SaO₂ level on days 3 through 11; deaths were recorded daily in these animals for 21 days.

Statistical Evaluation: Increase in survivor number was evaluated by chi square analysis with Yates' correction. Mean survival time increases and differences in SaO₂, lung weight and lung virus titers were analyzed by t-test. Lung score differences were evaluated by ranked sum analysis. In all cases, differences between drug-treated and saline-treated controls were studied.

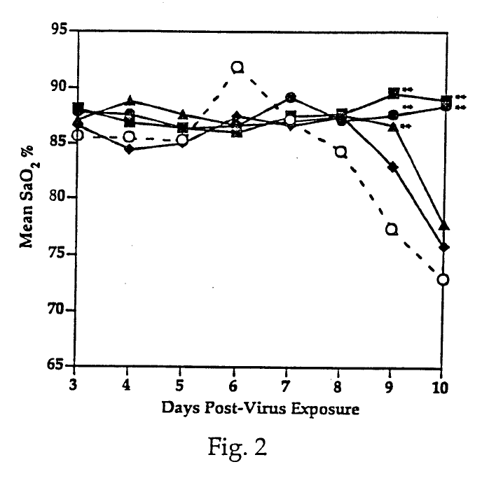

The results of the i.p. dosing experiment are summarized in Table I and in FIGS. 1 and 2. While in this model both compounds were significantly inhibitory at the high dose used, 203 treatment also resulted in significant survivors at a dose of 10 mg/kg/day. SaO₂ decline was particularly inhibited by both compounds at the 50 mg/kg/day dose, and again GG167 appeared to also prevent this decline at 10 and even 2 mg/kg/day. The lung score data appear to show the same trend of GG167 being effective at more than one dose. Some erraticism was seen in lung weights, with lungs taken from the mice receiving the highest dose of GG167 having a greater mean weight than the saline-treated controls.

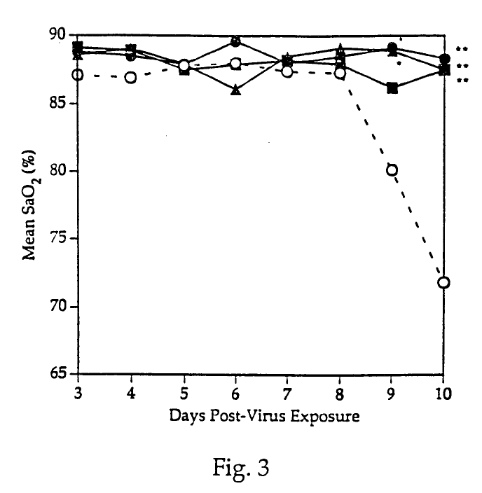

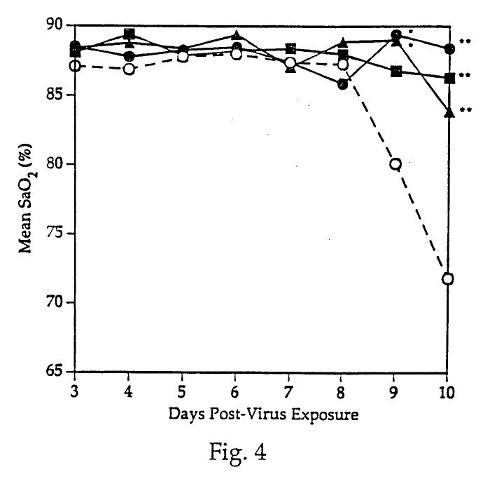

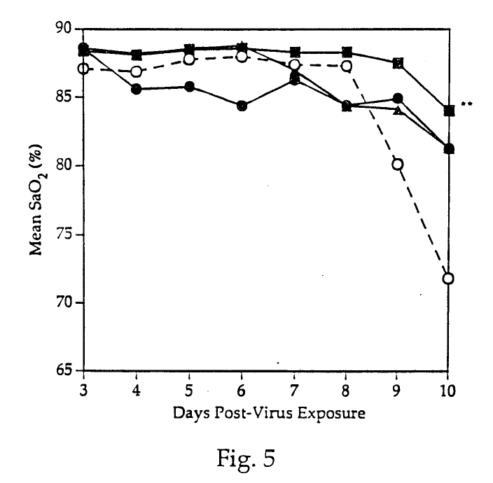

The p.o. dosing study is summarized in Table II, with daily SaO₂ values shown in FIGS. 3-5. Oral treatment with all three drugs in this model was significantly inhibitory to the influenza virus infection, preventing death, lowering lung scores and infection-associated lung weights, and inhibiting the usual decline in SaO₂.

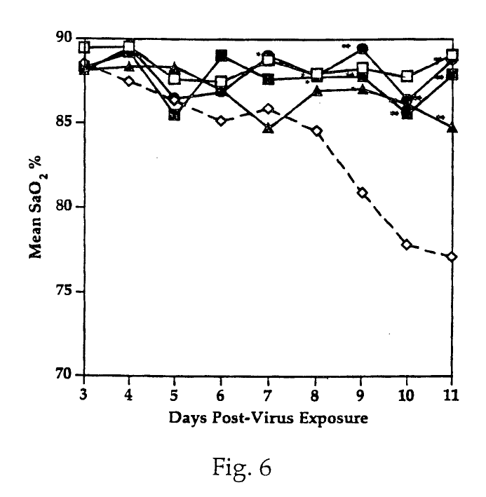

The p.o. low dose study results are summarized in Table III and in FIGS. 6-8. In this experiment, the infection was lethal to 14 of 16 saline-treated animals, the mean survival time being 9.6 days in this group. While all three compounds exhibited some degree of inhibitory effect on the virus infection, 262 (the ethyl ester prodrug) was the most effective at every dose as evidenced by number of survivors, mean survival time, and prevention of SaO₂ decline.

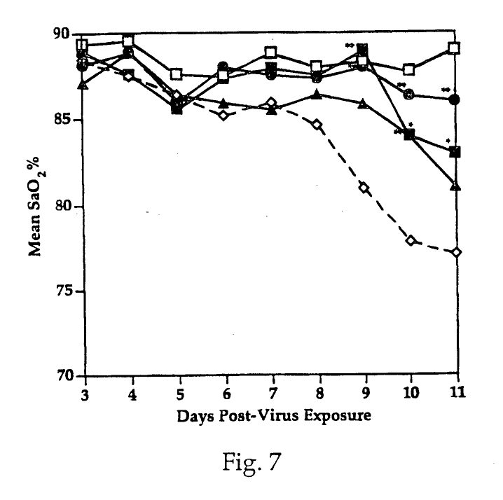

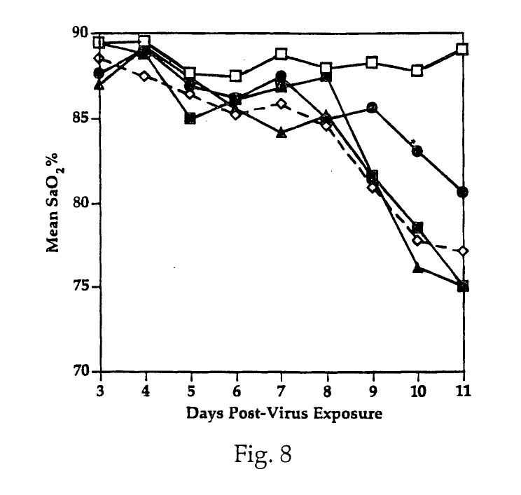

Table III shows the mean SaO₂% for all assay time taken together. The daily values for each compound are graphically represented in FIGS. 6 through 8. FIG. 6 illustrates the SaO₂ data with the highest concentrations of each compound; FIG. 7 shows the values at the median dose of each compound, and the SaO₂ values for the low dose of each compound are compared in FIG. 8.

Table III and FIGS. 6-8 indicate that while all three compounds were active orally against an experimentally induced influenza A (H1N1) virus infection, 262 was considered most effective. It was not determined whether the improved antiviral potency of 262 was unaccompanied with a concomitant increased animal toxicity, but this is unlikely since its greater efficacy is expected to be a result of its elevated oral bioavailability.

TABLE I
Comparison of the Effect of 203 and GG167 Administered
i.p.^{a} to Influenza A (H1N1) Virus-Infected Mice

| | | Infected, Treated | | | | |
|---|---|---|---|---|---|---|
| | | | | | Mean Lung | |
| | Dosage | | | | Parameters^{d} | |
| | (mg/kg/ | Surv/ | Mean Surv. | Mean | | Weight |
| Compound | day) | Total | Time^{b}(days) | SaO₂^{c} % | Score | mg |
| 203 | 50 | 8/8** | >21.0** | 87.2** | 0.7* | 173* |
| | 10 | 3/8* | 10.8 | 84.7 | 2.5 | 217 |
| | 2 | 0/7 | 12.6 | 84.4 | 2.0 | 203 |
| | 0.5 | 0/8 | 11.1 | 85.2* | 2.0 | 230 |
| GG167 | 50 | 8/8** | >21.0** | 87.6** | 0.7* | 230 |
| | 10 | 7/8** | 15.0 | 87.5** | 1.7 | 170* |
| | 2 | 1/8 | 12.6 | 86.0** | 1.3 | 213 |
| | 0.5 | 0/8 | 12.3 | 84.5 | 2.3 | 227 |
| Saline | −0/16 | 11.0 | 82.9 | 2.0 | 220 | |

TABLE II
Comparison of the Effect of Orally Administered^{a} 203, GG167
and Ribavirin on Influenza A (H1N1) Virus Infections in Mice.

| | | Infected, Treated | | | | |
|---|---|---|---|---|---|---|
| | | | | | Mean Lung | |
| | Dosage | | | | Parameters^{d} | |
| | (mg/kg/ | Surv/ | Mean Surv.^{b} | Mean | | Weight |
| Compound | day) | Total | Time (days) | SaO₂^{c} % | Score | (mg) |
| 203 | 250 | 8/8** | >21.0** | 87.9* | 0.8** | 160** |
| | 50 | 8/8** | >21.0** | 87.9* | 1.3* | 200 |
| | 10 | 4/8* | 12.8* | 87.7* | 1.3* | 240 |
| GG167 | 250 | 8/8** | >21.0** | 88.6* | 0.3** | 163** |
| | 50 | 8/8** | >21.0** | 88.0* | 1.5* | 187* |
| | 10 | 5/7* | 10.5 | 85.2 | 1.5* | 250 |
| Ribavirin | 100 | 8/8** | >21.0** | 88.2* | 0.3** | 140** |
| | 32 | 6/8* | 13.0 | 88.0* | 0.8** | 163** |
| | 10 | 3/8 | 11.0 | 86.4 | 2.2 | 267 |
| Saline | — | 1/16 | 10.9 | 84.5 | 2.4 | 203 |

TABLE III
Comparison of the Effect of Orally Administered^{a}
260, 262 and GG167 on Influenza A (H1N1) Virus
Infections in Mice.

| Com- | Dosage | Surv/ | | Mean Surv. | Mean |
|---|---|---|---|---|---|
| pound | (mg/kg/day) | total | % Survivors | Time^{b} (days) | SaO₂^{c (%)} |
| 260 | 10 | 6/8** | 75** | 13.5** | 87.6* |
| | 1 | 3/5 | 38 | 11.8 | 86.8 |
| | 0.1 | 0/8 | 0 | 10.0 | 84.3 |
| 262 | 10 | 8/8*** | 100*** | >21.0** | 88.1** |
| | 1 | 7/8*** | 88*** | 14.0** | 87.4* |
| | 0.1 | 2/8 | 25 | 11.1** | 85.7 |
| GG167 | 10 | 5/8* | 63* | 12.3** | 86.9 |
| | 1 | 2/8 | 25 | 11.7** | 85.7 |
| | 0.1 | 0/8 | 0 | 9.8 | 83.5 |
| Saline | 0 | 2/16 | 13 | 9.6 | 83.8 |

Footnotes for Tables I-III
^{a}Bid x 5 beginning 4 hr pre-virus exposure.
^{b}Animals dying on or before day 21.
^{c}Mean of values determined on days 3-10.
^{d}Determined on day 6.
*P<0.05,
**P<0.01,
***P<0.001 compared to saline-treated controls

Surprisingly, the foregoing demonstrates that in this model the oral or i.p. administration of GG167 was effective in practical therapeutic doses at reducing mortality in influenza-infected mice, despite the conclusion of Ryan et al. (“Antimicrob. Agents Chemother.”, 38(10):2270-2275) [1994]) that “it is likely that the relatively poor in vivo activity seen with GG167 in mice following intraperitoneal administration, despite good bioavailability, is due to its rapid clearance from the plasma, permitting poor penetration into respiratory secretions, coupled with its inability to penetrate and persist inside cells. Similarly, the poor efficacy following oral dosing is probably a consequence of poor oral bioavailability in addition to these other factors.” (p.2274). These observations are consistent with Von Izstein et al., WO 91/16320, WO 92/06691 and U.S. Pat. No. 5,360,817, which cover or are directed specifically to GG167. These patent documents are devoid of any teaching or suggestion to administer GG167 by any other route than intranasal. However, intranasal administration is believed to be inconvenient and costly in some circumstances. It would be advantageous if more facile routes of administration could be employed for GG167 and its related compounds set forth in WO 91/16320, WO 92/06691 and U.S. Pat. No. 5,360,817.

Thus, an embodiment of this invention is a method for the treatment or prophylaxis of influenza virus infection in a host comprising administering to the host, by a route other than topically to the respiratory system, a therapeutically effective dose of an antivirally active compound having formula (X) or (Y)

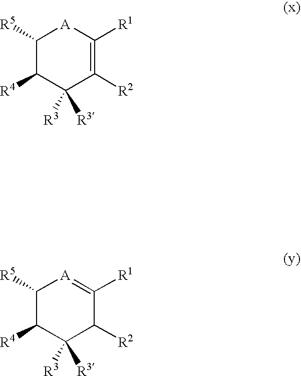

where in general formula (x), A is oxygen, carbon or sulphur, and in general formula (y), A is nitrogen or carbon;

R¹ denotes COOH, P(O)(OH)₂, NO₂, SOOH, SO₃H, tetrazol, CH₂CHO, CHO or CH(CHO)₂,

R² denotes H, OR⁶, F, Cl, Br, CN, NHR⁶, SR⁶, or CH₂X, wherein X is NHR⁶, halogen or OR⁶ and

R⁶ is hydrogen; an acyl group having 1 to 4 carbon atoms; a linear or cyclic alkyl group having 1 to 6 carbon atoms, or a halogen-substituted analogue thereof; an allyl group or an unsubstituted aryl group or an aryl substituted by a halogen, an OH group, an NO₂ group, an NH₂ group or a COOH group,

R³ and R³′ are the same or different, and each denotes hydrogen, CN, NHR⁶, N₃, SR⁶, ═N—OR⁶, OR⁶, guanidino,

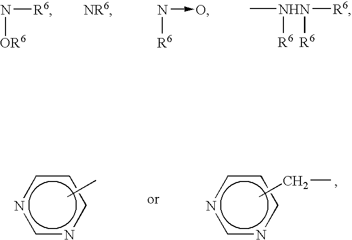

R⁴ denotes NHR⁶, SR⁶, OR⁶, COOR⁶, NO₂, C(R⁶)₃, CH₂COOR⁶, CH₂NO₂ or CH₂NHR⁶, and

R⁵ denotes CH₂YR⁶, CHYR⁶CH₂YR⁶ or CHYR⁶CHYR⁶CH₂YR⁶, where Y is O, S, NH or H, and successive Y moieties in an R⁵ group are the same or different,

and pharmaceutically acceptable salts or derivatives thereof, provided that in general formula (x)

(i) when R³ or R³′ is OR⁶ or hydrogen, and A is oxygen or sulphur, then said compound cannot have both

(a) an R² that is hydrogen and

(b) an R⁴ that is NH-acyl, and

(ii) R⁶ represents a covalent bond when Y is hydrogen, and that in general formula (y),

(i) when R³ or R³′ is OR⁶ or hydrogen, and A is nitrogen, then said compound cannot have both

(a) an R² that is hydrogen, and

(b) an R⁴ that is NH-acyl, and

(ii) R⁶ represents a covalent bond when Y is hydrogen.

The compounds of formulas x and y are more fully described in WO 91/16320, at page 3, line 23 to page 7, line 1, WO 92/06691 and U.S. Pat. No. 5,360,817, x and y are described therein as “I” and “Ia”, respectively.

For the purposes herein, administration by a route “other than topically to the respiratory tract means” does not exclude administration of compound by buccal or sublingual routes, and does not exclude incidental adsorption of compound in the esophagus during oral, buccal or sublingual administration, provided however, that such as buccal, oral, sublingual or esophageal adsorption is not incidental to administration to the lungs or nasal passages by inhalers or the like. Usually, compound is administered as a formed article, a slurry or a solution.

In typical embodiments of this invention, the compound is GG167, the host is an animal other than mice (such as ferrets or humans), the route of administration is oral, and the objective of treatment and prophylaxis is reduction in mortality. Optionally, a prodrug of the compound of formula (X) or (Y) is employed, although as shown above it is not necessary to do so to achieve antiviral effect by oral administration. As prodrugs of GG167 and its co-disclosed compounds, any of the esters, amides or other prodrugs described elsewhere herein for the compounds of this invention are suitable for use with the analogous groups of the compounds of formula (X) and (Y), e.g., carboxyl esters or amides.

The therapeutically effective dose of GG167 and its related compounds, when administered by oral or other non-nasal administration routes, will be determined by the ordinarily skilled clinician in light of the considerations set forth in connection with dosing the compounds of this invention. For the most part the principal considerations are the route of administration and the host species. In general, larger doses will be required as one proceeds from intravenous to subcutaneous to oral administration routes, and in accord with conventional pharmacologic scaling principles as one proceeds to larger animals. Determination of therapeutically active doses is well within the ordinary skill in the art, but in general the doses will be substantially the same as those employed for the compounds of this invention.

### Example 122

Each of the reactions shown in Table 50 were preformed according to Scheme 50. The preformed reactions are indicated with a “3”. Unless otherwise indicated in Table 50, steps AA, AB and AC were preformed according to Examples 92, 93 and 94, respectively, and step AD was preformed according to the combination of Examples 112 and 113.

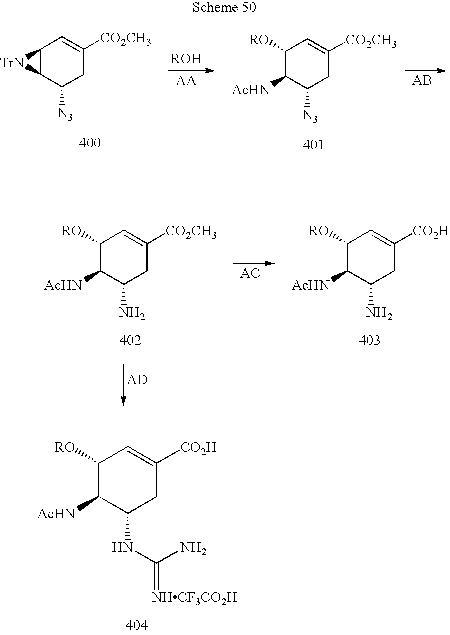

TABLE 50

| ROH | AA | AB | AC | AD |
|---|---|---|---|---|
| 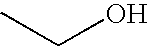 | 3 | 3 a,b | 3 c | |
| 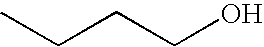 | 3 | 3 a,d | 3 c,e | 3 |
| 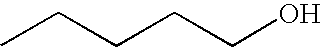 | 3 | 3 | 3 | |
| 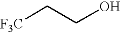 | 3 | 3 | 3 | |
| 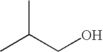 | 3 | 3 d | 3 c | 3 |
| 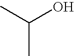 | 3 | 3 f | 3 | |
| 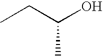 | 3 g | 3 | 3 | 3 |
| 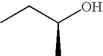 | 3 g | 3 | 3 | 3 |
| 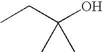 | 3 | 3 | 3 | |
| 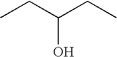 | 3 | 3 | 3 | 3 |
| 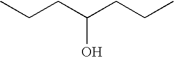 | 3 | 3 | 3 | |
| 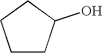 | 3 | 3 h | 3 | 3 |
| 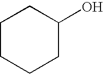 | 3 | 3 | 3 | |
| 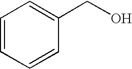 | 3 | 3 b,d | 3 | 3 |
| 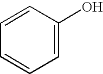 | 3 i,j | 3 | 3 | |
| 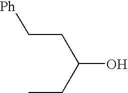 | 3 k,l | 3 | 3 | |
| 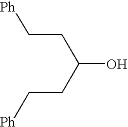 | 3 k | 3 | 3 | |
| 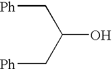 | 3 k | 3 | 3 | |

a ester hydrolysis prior to azide reduction
b azide reduction using Ph₃P at room temperature
c ester hydrolysis using aqueous KOH/MeOH
d azide reduction using polymer-support Ph₃P at room temperature
e isolated as the HCl salt
f azide reduction using Ph₃P in MeOH/THF/H₂O
g diastereomeric mixture, major diastereomer indicated
h azide reduction also performed with Me₃P
i aziridine opening performed at 55° C.
j C-alkylated products were isolated
k alcohol was not evaporated prior to acylation
l diastereomeric mixture, separated by chromatography/recrystallization

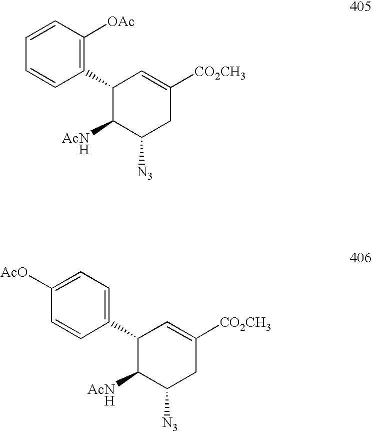

### Example 123

Trifluroacetamide 340: To a solution of amine 228 (100 mg, 0.34 mmol) in CH₂Cl₂ (3.5 mL) at 0° C. was added pyridine (41 μL, 0.51 mmol) and trifluroacetic anhydride (TFAA) (52 μL, 0.37 mmol) and the solution was stirred for 45 min at which time additional TFAA (0.5 eq) was added. After 15 min the reaction was evaporated under reduced pressure and the residue was partitioned between ethyl acetate and 1M HCl. The organic phase was washed with saturated NaHCO₃, saturated NaCl, and was dried (MgSO₄), filtered, and evaporated. The residue was chromatographed on silica gel (2/1-hexane/ethyl acetate) to afford trifluoroacetamide 340 (105 mg, 78%): ¹H NMR (CDCl₃) δ 8.64 (d, 1H, J=7.7), 6.81 (s, 1H), 6.48 (d, 1H, J=8.2), 4.25-4.07 (m, 3H), 3.75 (s, 3H), 3.37 (m, 1H), 2.76 (dd, 1H, J=4.5, 18.7), 2.54 (m, 1H), 1.93, (s, 3H), 1.48 (m, 4H), 0.86 (m, 6H).

### Example 124

N-Methyl trifluoroacetamide 341: To a solution of trifluroacetamide 340 (90 mg, 0.23 mmol) in DMF (2 mL) at 0° C. was added sodium hydride (10 mg, 60% dispersion in mineral oil, 0.25 mmol). After 15 min at 0° C., methyl iodide (71 μL, 1.15 mmol) was added and the reaction was stirred for 2 h at 0° C. and for 1 h at room temperature. Acetic acid (28 μL) was added was the solution was evaporated. The residue was partitioned between ethyl acetate and water. The organic phase was washed with saturated NaCl, dried (MgSO₄), filtered, and evaporated. The residue was chromatographed on silica gel (1/1-hexane/ethyl acetate) to afford N-methyl trifluoroacetamide 341 (81 mg, 87%) as a colorless glass: ¹H NMR (CDCl₃) δ 6.80 (s, 1H), 6.26 (d, 1H, J=9.9), 4.67 (m, 1H), 4.32 (m, 1H), 4.11 (m, 1H), 3.78 (s, 3H), 3.32 (m, 1H), 3.07 (br s, 3H), 2.60 (m, 2H), 1.91 (s, 3H), 1.48 (m, 4H), 0.87 (m, 6H).

### Example 125

N-Methyl amine 342: To a solution of N-methyl trifluoroacetamide 341 (81 mg, 0.20 mmol) in THF (3 mL) was added 1.04 N KOH (480 μL, 0.50 mmol) and the mixture was stirred at room temperature for 14 h. The reaction was acidified with IR 120 ion exchange resin to pH˜4. The resin was filtered, washed with THF, and the filtrate was evaporated. The residue was dissolved in 10% TFA/water (5 mL) and was evaporated. The residue was passed through a column (1.5×2.5 cm) of C-18 reverse phase silica gel eluting with water. Product fractions were pooled and lyophilized to afford N-methyl amine 342 (46 mg, 56%) as a white solid: ¹H NMR (D₂O) δ 6.80 (s, 1H), 4.31 (br d, 1H, J=8.8), 4.09 (dd, 1H, J=8.9, 11.6), 3.53 (m, 2H), 2.98 (dd, 1H, J=5.4, 16.9), 2.73 (s, 3H), 2.52-2.41 (m, 1H), 2.07 (s, 3H), 1.61-1.39 (m, 4H), 0.84 (m, 6H.

### Example 126

Compound 346: To a solution of epoxide 345 (13.32 g, 58.4 mmol) in 8/1-MeOH/H₂O (440 mL, v/v) was added sodium azide (19.0 g, 292.0 mmol) and ammonium chloride (2.69 g, 129.3 mmol) and the mixture was refluxed for 15 h. The reaction was cooled, concentrated under reduced pressure and partitioned between EtOAc and H₂O. The organic layer was washed successively with satd. bicarb, brine and dried over MgSO₄. Concentration in vacuo followed by flash chromatography on silica gel (30% EtOAc in hexanes) gave 11.81 g (75%) of azido alcohol 346 as a viscous oil. ¹H NMR(300 MHz, CDCl₃) δ 6.90-6.86 (m, 1H); 4.80 (s, 2H); 4.32 (bt, 1H, J=4.2 Hz); 4.22 (q, 2H, J=7.2 Hz); 3.90-3.74 (overlapping m, 2H); 3.44 (s, 3H); 2.90 (d, 1H, J=6.9 Hz); 2.94-2.82 (m, 1H); 2.35-2.21 (m, 1H); 1.30 (t, 3H, J=7.2 Hz).

### Example 127

Compound 347: To a solution of ethyl ester 346 (420 mg, 1.55 mmol) in dry THF (8.0 mL) cooled to −78° C. was added DIBAL (5.1 mL of a 1.0 M solution in toluene) dropwise via syringe. The bright yellow reaction mixture was stirred at −78° C. for 1.25 h and then slowly hydrolyzed with the slow addition of MeOH (1.2 mL). Volatiles were removed under reduced pressure and the residue partitioned between EtOAc and cold dilute HCl. The organic layer was separated and the aqueous layer back extracted with EtOAc. The organic layers were combined and washed successively with satd. bicarb, brine and dried over MgSO₄. Concentration in vacuo followed by flash chromatography on silica gel (20% hexanes in EtOAc) gave 127 mg (36%) of the diol 347 as a colorless viscous oil. ¹H NMR(300 MHz, CDCl₃) δ 5.83-5.82 (m, 1H); 4.78 (s, 2H); 4.21 (bt, 1H, J=4.4 Hz); 4.06 (bs, 2H); 3.85-3.65 (overlapping m, 2H); 3.43 (s, 3H); 3.18 (d, 1H, J=8.1 Hz); 2.51 (dd, 1H, J=5.5, 17.7 Hz); 2.07-1.90 (m, 1H); 1.92 (bs, 1H).

### Example 128

Methyl ester 600: Prepared in 51% overall yield from D-(−)-quinic acid according to the procedure of Frost, J. W., et. al. “J. Org. Chem.” 61:3897 (1996).

### Example 129

Ketone 601: To a slurry of diol 600 (15.0 g, 46.9 mmol), pyridine (13.7 mL), celite (equal volume to PCC) in dichloromethane (200 mL) was added PCC (40.5 g, 187.9 mmol) in portions and the reaction was stirred at room temperature for 21 h. Excess PCC was destroyed with the addition of excess 2-propanol. After stirring for an additional 30 min the reaction mixture was diluted with diethyl ether, filtered through a pad of celite and washed with ethyl acetate. The organic layer was then passed through a short column of silica gel and eluted with ethyl acetate. Concentration under reduced pressure gave a yellow solid which was recrystallized from methanol/ethyl acetate/hexanes to give 10.9 g (74%) of ketone 601 as a crystalline powder. HRMS (FAB): Calcd for C₁₄H₂₂O₈ (MLi+) 325.1474, found 325.1471.

### Example 130

Olefin 602: To a slurry of butyltriphenylphosphonium bromide (16.6 g, 41.6 mmol) in dry THF (150 mL) cooled to 0° C. was added n-BuLi (26.0 mL of a 1.61 M solution in hexane) dropwise. After stirring at 0° C. for 20 min the mixture was warmed to room temperature, stirred for 5 min and recooled to 0° C. To this bright orange solution was added a solution of 601 (6.0 g, 18.9 mmol) in dry THF (75.0 mL) via cannula. The reaction mixture was warmed to room temperature, stirred for 10 min and then gently refluxed for 2.5 h. The reaction mixture was cooled, saturated NaHCO₃ was added and diluted with ethyl acetate. The organic layer was separated, washed with brine and dried over MgSO₄. Concentration under reduced pressure followed by flash column chromatography on silica gel (30% hexanes in ethyl acetate) gave 5.5 g (81%) of 602 as a viscous pale oil consisting of a 4:1 mixture of olefin isomers.

### Example 131

Triethylsilyl ether 603: To a solution of 602 (5.5 g, 15.37 mmol) in dichloromethane (125 mL) cooled to 0° C. was added 2,6-lutidine (3.6 mL) followed by the dropwise addition of triethylsilyl trifluoromethanesulfonate (5.35 mL, 23.66 mmol). The reaction mixture was slowly warmed to room temperature and stirred for 15 h. Volatiles were removed under reduced pressure and the crude residue was partitioned between diethyl ether and water. The organic layer was washed with dilute HCl, saturated NaHCO₃, brine and dried over MgSO₄. Concentration under reduced pressure followed by flash column chromatography on silica gel (20% ethyl acetate in hexanes) gave 6.78 g (93%) of 603 as a mobile liquid.

### Example 132

Butyl cyclohexyl ester 604: To a degassed solution of olefin 603 (6.78 g, 14.34 mmol) in ethanol (140 mL) was added 10% palladium on carbon (5.0 g). The reaction mixture was then stirred under an atmosphere of hydrogen gas (1 atm via balloon) at room temperature for 22 h. The reaction was filtered through a celite pad and washed with hot methanol. Concentration under reduced pressure followed by flash column chromatography on silica gel (10% ethyl acetate in hexanes) gave 5.44 g (80%) of 604 as a colorless oil.

### Example 133

Alcohol 605: A solution of tetrabutylammonium fluoride (17.1 mL of a 1.0 M solution in THF) was added dropwise to a solution of 604 (5.44 g, 11.46 mmol) in THF (50 mL) at room temperature. After 45 min the bulk of the THF was removed under reduced pressure and the crude reaction was partitioned between diethyl ether and water. The organic layer was washed with saturated ammonium chloride, water, brine and dried over MgSO₄. Concentration under reduced pressure followed by flash column chromatography on silica gel (20% ethyl acetate in hexanes ) gave 3.18 g (77%) of 605 as a colorless viscous oil.

### Example 134

Olefin 606: To a solution of alcohol 605 (3.18 g, 8.82 mmol) in pyridine (39 mL) and dry dichloromethane (35 mL) cooled to −78° C. was added sulfuryl chloride (1.07 mL, 13.32 mmol) dropwise via syringe. The reaction mixture was slowly warmed to −40° C. over a 30 min period and maintained between −40°-30° C. for 30 min. The reaction was recooled to −78° C. and methanol (1.0 mL) was added. The reaction was then slowly warmed to room temperature over a 3 h period and then diluted with diethyl ether. The organic layer was washed sequentially with water, dilute HCl, water, saturated NaHCO₃, brine and dried over MgSO₄. Concentration under reduced pressure followed by flash column chromatography on silica gel (25% ethyl acetate in hexanes) gave 2.73 g (90%) of 606 as a colorless viscous oil which is contaminated with 3% of the isomeric cyclohexene carboxylate.

### Example 135

Diol 607: A solution of 606 (2.73 g, 7.97 mmol) in dichloromethane (58 mL) was treated with 40% aqueous trifluoroacetic acid (37 mL) at room temperature for 14 h. Volatiles were removed under reduced pressure and the residue was partitioned between diethyl ether and water. The organic layer was cautiously washed with saturated NaHCO₃, water, brine and dried over MgSO₄. Concentration under reduced pressure followed by flash column chromatography on silica gel (10% hexanes in ethyl acetate) gave 1.36 g (75%) of 607 as a viscous oil.

### Example 136

Mesylates 608 and 609: To a solution of diol 607 (1.06 g, 4.64 mmol) and triethyl amine (1.31 mL) in dichloromethane (25 mL) cooled to −78° C. was added dropwise methanesulfonyl chloride (360 μL, 4.64 mmol). The reaction was stirred at −78° C. for 1 h and then slowly warmed to 0° C. over a 1 h period. After an additional 1 h at this temperature, the reaction was diluted with diethyl ether and washed with water, saturated NaHCO₃, brine and dried over MgSO₄. Concentration under reduced pressure followed by flash column chromatography on silica gel (20% ethyl acetate in hexanes) gave 1.23 g (87%) of 608 and 609 as an inseparable mixture in a 6:1 ratio, respectively.

### Example 137

Epoxide 610: To a solution of a 6 to 1 mixture of 608 and 609 (1.23 g, 4.02 mmol) in dry THF (20 mL) cooled to 0° C. was added DBU (601 μL, 4.02 mmol). The ice bath was removed and the reaction stirred at room temperature for 18 h. The reaction was diluted with diethyl ether and washed with water, brine and dried over MgSO₄. Concentration under reduced pressure followed by flash column chromatography on silica gel (20% ethyl acetate in hexanes) gave 490 mg (58%) of pure epoxide 610 as a mobile liquid and 100 mg (13%) of methyl-3-butyl benzoate 611 as an oil. Anal. Calcd for C₁₂H₁₈O₃: C, 68.55; H, 8.63. Found: C, 68.29; H, 8.52.

### Example 138

Azido alcohols 612 and 613: A solution of 610 (490 mg, 2.33 mmol), sodium azide (764 mg, 11.75 mmol) and ammonium chloride (281 mg, 5.25 mmol) in methanol/water (8:1, 17.0 mL) was gently refluxed for 15 h. The cooled reaction mixture was concentrated under reduced pressure and partitioned between diethyl ether and water. The organic layer was washed with brine and dried over MgSO₄. Concentration under reduced pressure followed by flash column chromatography on silica gel (20% ethyl acetate in hexanes) gave 562 mg (95%) of 612 and 613 as an inseparable mixture in a 2:1 ratio, respectively.

### Example 139

Azido mesylates 614 and 615: To a solution of 612 and 613 (642 mg, 2.54 mmol), triethyl amine (1.8 mL) and catalytic DMAP in dichloromethane (15 mL) cooled to 0° C. was added dropwise methanesulfonyl chloride (232 μL, 3.00 mmol). The reaction was stirred at 0° C. for 1.5 h and then at room temperature for 30 min. The reaction was diluted with diethyl ether and washed with water, dilute HCl, saturated NaHCO₃, brine and dried over MgSO₄. Concentration under reduced pressure gave a yellow liquid which was passed through a short plug of silica gel eluting with 25% ethyl acetate in hexanes to give 840 mg (100%) of 614 and 615 as an inseparable mixture.

### Example 140

Aziridine 616: To a solution of 614 and 615 (840 mg, 2.53 mmol) in dry THF (20 mL) was added triphenyl phosphine (750 mg) in portions at room temperature. After 2.5 h triethyl amine (550 μL) and water (5.50 mL) were added and the reaction stirred at room temperature for 16 h. Volatiles were removed under reduced pressure and the residue diluted with ethyl acetate. The organic layer was washed with water, saturated NaHCO₃, brine and dried over MgSO₄. Concentration under reduced pressure followed by flash column chromatography on silica gel (5% methanol in ethyl acetate) gave 375 mg (71%) of 616 as a viscous oil.

### Example 141

Azido amine 617: A solution of 616 (354 mg, 1.70 mmol), sodium azide (555 mg, 8.54 mmol) and ammonium chloride (182 mg, 3.40 mmol) in dry DMF (8.0 mL) was heated at 80° C. for 17 h. The bulk of the DMF was removed under reduced pressure and the residue partitioned between diethyl ether and water. The organic layer was washed with water, brine and dried over MgSO₄. Concentration under reduced pressure gave a yellow liquid which was passed through a short plug of silica gel eluting with ethyl acetate to give 380 mg (86%) of 617 as a yellow liquid which was used immediately for the next reaction.

### Example 142

N-acetyl azide 618: The crude amine 617 (380 mg, 1.51 mmol) in dry pyridine (3.0 mL) and dichloromethane (7.0 mL) was treated with acetyl chloride (173 μL, 2.40 mmol) at 0° C. After 40 min the reaction was warmed to room temperature and stirred for 5 min. Volatiles were removed under reduced pressure and the residue was partitioned between diethyl ether and water. The organic layer was washed with dilute HCl, saturated NaHCO₃, brine and dried over MgSO₄. Concentration under reduced pressure followed by flash column chromatography on silica gel (20% hexanes in ethyl acetate) gave 349 mg of an off-white solid which was recrystallized from ethyl acetate and hexanes to give 304 mg (68%) of 618 as colorless needles.

### Example 143

N-acetyl amino ester 619: A solution of 618 (292 mg, 0.99 mmol) and triphenyl phosphine (393 mg, 1.50 mmol) in water (1.8 mL) and THF (15 mL) was heated at 50° C. for 10 h. The reaction was evaporated to dryness, applied to a silica gel column and eluted with 40% methanol in ethyl acetate to give 250 mg (93%) of 619 as a pale gummy solid.

### Example 144

Amino acid 620: A solution of 619 (142 mg, 0.53 mmol) in THF (2.0 mL) was treated at room temperature with aqueous KOH (770 μL of a 1.039 M solution) for 3.5 h and then acidified to pH=3.0 with Amberlite IR-120 (H⁺) ion-exchange resin. The reaction was filtered and the resin washed with water and methanol. Concentration under reduced pressure gave a pale solid which was purified by C₈ reverse phase column chromatography eluting with water. Fractions containing the desired product were pooled and evaporated to give 87 mg (65%) of 620 as a colorless powder.

### Example 145

Azido propyl ester 265: To a solution of carboxylic acid 260 (55 mg, 0.18 mmol), 1-propanol (67 μL, 0.89 mmol), and catalytic DMAP in CH₂Cl₂ (1.0 mL) was added diisopropyl carbodiimide (31 μL, 0.19 mmol) dropwise at room temperature. After stirring for 1 h the reaction was concentrated and purified by flash chromatography on silica gel (50% hexanes in ethyl acetate) to give 53 mg (85%) of 265 as a colorless crystalline solid.

### Example 146

Amino propyl ester 266: Triphenyl phosphine (65 mg, 0.25 mmol) was added in one portion to a solution of 265 (53 mg, 0.15 mmol) in THF (4.0 mL) and water (300 μL). The reaction was then heated at 50° C. for 10 h, cooled and concentrated in vacuo to give a pale white solid. Purification of the crude solid by flash chromatography on silica gel (50% methanol in ethyl acetate) gave a pale oil which was evaporated from 3 N HCl to give a solid which was purified by C₁₈ reverse phase column chromatography eluting with water. Fractions containing the desired product were pooled and lyophilized to give 41 mg (75%) of 266 as a colorless powder.

### Example 147

Sulfide 700 was made from shikimic acid according to a literature procedure (Robert H. Rich, Brian M. Lawrence, Paul A. Bartlett, “J. Org. Chem.”, 59:693-694 (1994).

### Example 148

Sulfoxide 701: To a solution of sulfide 700 (16.0 g, 32.7 mmol) in CH₂Cl₂ (750 mL) at −45° C. was dropwise added a solution of m-chloroperoxybenzoic acid (8.5 g, 57-86%) in CH₂Cl₂ (250 mL) over a period of 0.5 h. The reaction was stirred at −40° C. for 1 h, then at room temperature for 0.5 h. The reaction mixture was evaporated to solid began to precipitate out, and then diluted with hexane. The solid was removed by filtration and the filtrate was evaporated. The residue was dissolved in ethyl acetate and washed with saturated NaHCO₃, dried (MgSO₄), filtered and evaporated. The crude product was purified by chromatography on silica gel (ethyl acetate/hexane) to give sulfoxide 701 (14.2 g, 86%, a mixture of diastereomers, ratio=2.2:1) as a colorless solid.

### Example 149

Vinyl Chloride 702: The sulfoxide 701 (14.0 g, 27.7 mmol) was refluxed in xylene (180 mL) for 50 min. The reaction mixture was cooled to room temperature and evaporated. The residue was chromatographed to afford vinyl chloride 702 (7.6 g, 79%) as an oil.

### Example 150

Triol 703: To a solution of vinyl chloride 702 (7.3 g, 20.9 mmol) in anhydrous methanol (80 mL) at room temperature was added sodium methoxide (0.3 mL, 25%, 1.3 mmol). The reaction was stirred at room temperature for 1 h, then quenched with HCl/CH₃OH (1.0 mL, 1.4M, 1.4 mmol). The reaction mixture was evaporated and the residue was treated with ethyl acetate/hexane to give triol 703 (4.6 g, 99%) as a colorless solid. Anal. Calcd for C₈H₁₁ClO₅^{.1}/₁₄NaCl: C, 42.36; H, 4.89; Cl, 16.75. Found: C, 42.29; H, 4.90; Cl, 16.56.

### Example 151

Acetonide 704: The mixture of triol 703 (4.6 g, 20.7 mmol), 2,2-dimethoxypropane (4.0 mL, 32.5 mmol) and acetone (50 mL) was stirred at room temperature for 1.5 h. The reaction mixture was evaporated, and fresh 2,2-dimethoxypropane (1.5 mL, 12.2 mmol) and acetone (30 mL) were added. The reaction was stirred for another 1.5 h. The reaction mixture was evaporated, and the crude product was filtered through a short plug of silica gel. The filtrate was evaporated to give acetonide 704 (5.4 g, 99%) as an oil, Anal. Calcd for C₁₁H₁₅ClO₅^{.1}/₄H₂O: C, 49.45; H, 5.85; Cl, 13.27. Found: C, 49.67; H, 5.82; Cl, 13.60.

### Example 152

Mesylate 705: To a solution of acetonide 704 (2.63 g, 10.0 mmol) in CH₂Cl₂ (30 mL) at 0° C. was added triethylamine (2.23 mL, 16 mmol), followed by methanesulfonyl chloride (1.16 mL, 15 mmol). The reaction was stirred at 0° C. for 1 h, then evaporated. The residue was partitioned between ethyl acetate and water. The aqueous phase was extracted with ethyl acetate. The combined organic phases were dried (MgSO₄), filtered and evaporated. The crude product was filtered through a short plug of silica gel. The filtrate was evaporated to give mesylate 705 (3.4 g, 100%) as an oil.

### Example 153

3-Pentyl Ketal 706: The mixture of mesylate 705 (3.4 g, 10.0 mmol) and perchloric acid (30 mg, 70%, 0.2 mmol) in 3-pentanone (40 mL) was stirred at 45° C. for 2 h. The reaction was evaporated and fresh 3-pentanone (40 mL) was added. The reaction was stirred for another 0.5 h, then evaporated. The crude product was filtered through a short plug of silica gel. The filtrate was evaporated to afford 3-pentyl ketal 706 (3.7 g, 100%) as an oil.

### Example 154

Mesylate Alcohol 707: To a solution of ketal 706 (1.68 g, 4.55 mmol) in CH₂Cl₂ (20 mL) at −5° C. was added borane-methyl sulfide complex (0.7 mL, 10M, 7.0 mmol), followed by trimethylsilyl trifluoromethanesulfonate (0.82 mL, 4.6 mmol). The resulted mixture was stirred at 0° C. for 1 h, then very slowly added saturated NaHCO₃ (1 drop/10 min. for the first 5 drops, 1 mL). The resulted mixture was filtered through a short plug of silica gel. The filtrate was evaporated and the residue was purified by chromatography on silica gel (ethyl acetate/hexane) to give a mixture of regio-isomers 707 and 708 (1.2 g, 71%, 8/9=3/2) as an oil.

### Example 155

Epoxide 709: A mixture of 707 and 708 (1.95 g, 5.26 mmol) was mixed with KHCO₃ (1.0 g, 10 mmol) in methanol (15 mL) and water (10 mL). The reaction was stirred at 50° C. for 1 h, then evaporated to remove methanol. The remained mixture was extracted with ethyl acetate. The combined extracts was dried (MgSO₄), filtered, evaporated. The residue was chromatographed to give epoxide 709 (0.88 g, 61%) as an oil.

### Example 156

Azide Alcohol 710: The mixture of epoxide 709 (0.95 g, 3.46 mmol), sodium azide (0,65 g, 10 mmol) and ammonium chloride (0,40 g, 7.5 mmol) in methanol (40 mL) and water (10 mL) was stirred at 65° C. for 18 h. The reaction mixture was diluted with water and evaporated to remove methanol, then extracted with ethyl acetate. The organic extracts were dried (MgSO₄), filtered and evaporated. The crude product was crystallized from hexane/ethyl acetate to afford azide alcohol 710 (0,8 g, 73%) as a colorless solid. Anal. Calcd for C₁₃H₂₀ClN₃O₄: C, 49.14; H, 6.34; N, 13.22; Cl, 11.16. Found: C, 49.14; H, 6.47; N, 13.21; Cl, 11.38.

### Example 157

Azide mesylate 711: To a solution of azide alcohol 710 (1.0 g, 3.15 mmol) in CH₂Cl₂ (20 mL) at 0° C. was added triethylamine (1.1 mL, 8.0 mmol), followed by methanesulfonyl chloride (0.5 mL, 6.5 mmol). The resulted mixture was stirred at 0° C. for 0.5 h, then at room temperature for another 0.5 h. The reaction was added 2 drops of water, then diluted with hexane and filtered through a short plug of silica gel. The filtrate was evaporated to give azide mesylate 711 (1.27 g, 100%) as an oil.

### Example 158

Azido phenethyl ester 800: To a solution of 260 (63 mg, 0.20 mmol), phenethyl alcohol (26 μL, 0.22 mmol), and DMAP (7.8 mg) in 1/1-CH₂Cl₂/THF (2 mL) was added diisopropylcarbodiimide (34 μL, 0.22 mmol) at room temperature. After stirring 4 h the solvent was evaporated and the residue was chromatographed on silica gel (1/1-hexane/ethyl acetate) to afford 800 (60 mg) as an oil which contained a trace of phenethyl alcohol. This material was used directly in the next step without any further purification.

### Example 159

Amino phenethyl ester 801: Triphenyl phosphine (55 mg, 0.21 mmol) was added in one portion to a solution of 800 (60 mg, 0.14 mmol) in THF (2 mL) and water (252 μL). The reaction was then heated at 50° C. for 10 h, cooled and evaporated. The residue was purified by silica gel chromatography (1/1-ethyl acetate/methanol) to afford 53 mg of an oil which was dissolved in 0.1N HCl (1 mL) and evaporated. The residue was dissolved in water and passed through a column of C₁₈ reverse phase silica gel to afford after lyophilization 801 (41 mg, 69%) as a white solid.

### Example 160

Azido butyl ester 802: To a solution of 260 (60 mg, 0.19 mmol), n-butanol (87 μL, 0.95 mmol), and DMAP (4 mg) in 2/1-CH₂Cl₂/THF (3 mL) was added diisopropylcarbodiimide (33 μL, 0.21 mmol) at room temperature. After stirring 2 h the solvent was evaporated and the residue was chromatographed on silica gel (1/1-hexane/ethyl acetate) to afford 802 (48 mg, 68%) as an oil.

### Example 161

Amino butyl ester 803: Triphenyl phosphine (51 mg, 0.19 mmol) was added in one portion to a solution of 802 (48 mg, 0.13 mmol) in THF (1.5 mL) and water (234 μL). The reaction was then heated at 50° C. for 10 h, cooled and evaporated. The residue was dissolved in ethyl acetate, dried (Na₂SO₄), filtered and evaporated. Purification of the residue by silica gel chromatography (1/1-ethyl acetate/methanol) afforded 38 mg of an oil which was dissolved in 0.1N HCl (2 mL) and evaporated. The residue was dissolved in water and passed through a column of C₁₈ reverse phase silica gel to afford after lyophilization 803 (23 mg, 47%) as a white solid.

### Example 162

1-Phenyl-3-pentanol 804: To a solution of ethylmagnesium bromide (75 mmol) in ether (325 mL) at 0° C. was added hydrocinnamaldehyde (6.71 g, 50 mmol) in ether (50 mL). The solution was stirred for 1 h and was allowed to warm to room temperature. The reaction solution was poured into ice-water (1000 mL) and the mixture was acidified to pH=3 with conc. HCl. The layers were separated and the aqueous phase was extracted with ether. The combined organic extracts were washed with saturated NaHCO₃, brine, and were dried (MgSO₄), filtered, evaporated. The crude product was distilled under high vacuum (bp 90-93° C.) to afford 804 (5.3 g, 64%) as a colorless oil.

### Example 163

1,5-diphenyl-3-pentanol 805: To a solution of phenethylmagnesium bromide (25 mL, 0.9M in THF) in ether (100 mL) at 0° C. was added hydrocinnamaldehyde (3.0 g, 22.5 mmol) in ether (30 mL). The solution was stirred for 5 min and was allowed to warm to room temperature stirring for 1 h. The reaction solution was poured into ice-water (200 mL) and the mixture was acidified to pH=3 with conc. HCl. The layers were separated and the aqueous phase was extracted with ether. The combined organic extracts were washed with saturated NaHCO₃, brine, and were dried (MgSO₄), filtered, evaporated. Chromatography on silica gel (4/1-hexane/ethyl acetate) gave a pale yellow oil (3.74 g) which solidified upon cooling. Recrystallization from hexane gave 805 (1.35 g, 25%) as white needles.

### Example 164

1,3-diphenyl-2-propanol 806: To a solution of 1,3-diphenylacetone (17.08 g, 81.2 mmol) in ethanol (100 mL) at 0° C. was added NaBH₄ (3.07 g, 81.2 mmol) and the mixture was stirred for 2 h. The reaction was acidified to pH=3 with 1N HCl and ethanol was evaporated. The reaction was diluted with water and the aqueous phase was extracted with several portions of ethyl acetate. The combined organic extracts were washed with saturated NaHCO₃, brine, dried (MgSO₄), filtered and evaporated to afford 806 (17 g, 99%) as a pale yellow oil.

### Example 165

Ether 807: To a solution of 183 (200 mg, 0.46 mmol) and 804 (1 mL) was added BF₃.OEt₂ (85 μL, 0.69 mmol) and the solution was heated at 75-80° C. for 1.25 h. After cooling to room temperature the reaction was diluted with pyridine (5 mL) cooled to 0° C., and treated with acetic anhydride (1.25 mL) and DMAP (50 mg). The reaction was stirred at 0° C. for 15 min. and then at room temperature for 14 h. The solvent was evaporated and the residue was partitioned between ethyl acetate and 1N HCl and the organic phase was washed again with 1N HCl. The combined aqueous washes were extracted with ethyl acetate, and the combined organic extracts were washed with saturated NaHCO₃, brine, dried (MgSO₄), filtered and evaporated. The residue was chromatographed on silica gel (1/1-hexane/ethyl acetate) to afford 807 (116 mg mg, 63%) as a mixture of diastereomers which was rechromatographed (2/1-hexane/ethyl acetate). Fractions containing the faster eluting diastereomer were combined to afford 807a (44 mg) as a solid which was recrystallized (hexane/ethyl acetate): mp 131-133° C. The slower eluting diastereomer was obtained as a solid which was recrystallized (hexane/ethyl acetate) to afford 807b (41 mg) as needles: mp 111-112° C.

### Example 166

Azidoesters 807a and 807b were treated with triphenylphosphine in a similar manner as described in Example 93 to afford amino esters 808a and 808b, which were treated with aqueous potassium hydroxide as described in Example 94 to afford amino acids 809a and 809b.

### Example 167

Ether 810: A solution of 183 (200 mg, 0.46 mmol) and 805 (750 mg, 3.1 mmol, mp 43-45° C.) was formed by gentle heating. To this solution was added BF₃.OEt₂ (85 μL, 0.69 mmol) and the solution was heated at 70-75° C. for 1.5 h. After cooling to room temperature the reaction was diluted with pyridine (2 mL) cooled to 0° C., and treated with acetic anhydride (660 μL, 7.0 mmol) and catalytic DMAP. The reaction was stirred at 0° C. for several min and then at room temperature for 16 h. The solvent was evaporated and the residue was partitioned between ethyl acetate and 1N HCl and the organic phase was washed again with 1N HCl. The combined aqueous washes were extracted with ethyl acetate, and the combined organic extracts were washed with saturated NaHCO₃, brine, dried (MgSO₄), filtered and evaporated. The residue was chromatographed on silica gel (1/1-hexane/ethyl acetate) to afford a solid residue which was recrystallized (hexane/ethyl acetate) to afford 810 (63 mg, 28%) as needles: mp 139-140° C.

### Example 168

Azidoester 810 was treated with triphenylphosphine in a similar manner as described in Example 93 to afford amino ester 811, which was treated with aqueous potassium hydroxide as described in Example 94 to afford amino acid 812.

### Example 169

Ether 813: To a solution of 183 (100 mg, 0.23 mmol) and 806 (1 mL) was added BF₃.OEt₂ (42 μL, 0.35 mmol) and the solution was heated at 70-75° C. for 1.25 h. After cooling to room temperature the reaction was diluted with pyridine (5 mL) cooled to 0° C., and treated with acetic anhydride (680 μL, 7.2 mmol) and catalytic DMAP. The reaction was stirred at 0° C. for several min. and then at room temperature for 15 h. The solvent was evaporated and the residue was partitioned between ethyl acetate and 1N HCl and the organic phase was washed again with 1N HCl. The combined aqueous washes were extracted with ethyl acetate, and the combined organic extracts were washed with saturated NaHCO₃, brine, dried (MgSO₄), filtered and evaporated. The residue was chromatographed (1/1-hexane/ethyl acetate) to afford 813 (57 mg, 55%) as a pale yellow solid: mp 132-133° C. (needles from hexane/ethyl acetate)

### Example 170

Azidoester 813 was treated with triphenylphosphine in a similar manner as described in Example 93 to afford amino ester 817, which was treated with aqueous potassium hydroxide as described in Example 94 to afford amino acid 815.

### Example 171

N-Boc aziridine 817: To a solution of 816 (700 mg, 3.1 mmol, prepared in a similar manner from quinic acid as described for methyl ester derivative 170) in CH₂Cl₂ (10 mL) was added di-tert-butyldicarbonate (1.0 g, 4.6 mmol) in CH₂Cl₂ (5 mL) and catalytic DMAP (10 mol%). After stirring for 45 min at room temperature the solvent was evaporated and the residue was directly purified by silica gel chromatography (3/1-hexane/ethyl acetate) to afford 817 (880 mg, 87%) as an oil.

### Example 172

Alcohol 818: To a solution of 817 (826 mg, 2.52 mmol) in DMF (20 mL) was added ammonium formate (1.59 g, 25.2 mmol) and the mixture was heated at 130° C. for 1 h. After a second addition of ammonium formate (1.59 g, 25.2 mmol) the reaction was heated for 1.5 h and was evaporated. The residue was partitioned between ethyl acetate and saturated NaHCO₃. The organic phase was washed with brine, dried (MgSO₄), filtered and evaporated. The residue was purified by silica gel chromatography (1/2-hexane/ethyl acetate) to afford 818 (556 mg, 64%) as a pale yellow solid.

### Example 173

Acetate 819: To a solution of 818 (500 mg, 1.45 mmol) in pyridine (10 mL) was added DMAP (20 mg, 0.16 mmol) and acetic anhydride (216 μL, 2.3 mmol). The solution was stirred for 1 h at room temperature and was evaporated. The residue was purified by silica gel chromatography (1/1-hexane/ethyl acetate) to afford 819 (557 mg, 94%) as a solid.

### Example 174

N-Trityl aziridine 820: A solution of 819 (459 mg, 1.18 mmol) in 1.24 M HCl in ethyl acetate (20 mL) was stirred at room temperature for 2.5 h. The solvent was evaporated to afford a white solid which was placed under high vacuum overnight. To a solution of the solid (315 mg) in CH₂Cl₂ (10 mL) at 0° C. was added trityl chloride (346 mg, 1.24 mmol) and Et₃N (354 μL, 2.54 mmol). The solution was stirred for 1.75 h at which time Et₃N (354 μL, 2.54 mmol) and methanesulfonyl chloride (105 μL, 1.36 mmol) were added. The reaction mixture was stirred at 0° C. for 1.5 h and was warmed to room temperature stirring for 5 h. The solvent was evaporated and the residue was partitioned between ether and water. The organic phase was washed with water and the combined aqueous washes were extracted with ether. The combined organic extracts were washed with brine, dried (MgSO₄), filtered and evaporated. Purification of the residue by silica gel chromatography (CH₂Cl₂) afforded 820 (440 mg, 83%) as a white foam.

### Example 175

Pentyl ether 821: To a solution of 820 (100 mg, 0.21 mmol) in 3-pentanol (2 mL) was added BF₃.OEt₂ (39 μL, 0.32 mmol) and the solution was heated at 75-80° C. for 1.5 h. After evaporation of the solvent, the residue was dissolved in pyridine (2 mL) and was treated with acetic anhydride (100 μL, 1.05 mmol) and DMAP. The reaction was stirred at room temperature for 14 h, evaporated and the residue was partitioned between ethyl acetate and IN HCl. The aqueous phase was extracted with ethyl acetate and the combined organic extracts were washed with saturated NaHCO₃, brine, dried (MgSO₄), filtered and evaporated. The residue was chromatographed on silica gel (1/1-ethyl acetate/CH₂Cl₂) to afford 821 (46 mg, 62%) as a solid.

### Example 176

Hydroxy acid 822: To a solution of 821 (42 mg, 0.12 mmol) in THF (2 mL) was added 1N KOH (260 μL, 0.27 mmol) and the mixture was stirred at room temperature for 5.5 h. The solution was acidified with Amberlite IR120 ion exchange resin (pH 3) and the resin was filtered and washed with THF. Sovent was evaporated to afford a residue which was dissolved in water and chromatographed on C₈ reverse phase silica gel eluting with water. The water was evaporated and the residue was evaporated from methanol to give 822 (29 mg, 85%) as a solid.

### Example 177

Methyl ether 823: To a solution of 816 (200 mg, 0.88 mmol) in methanol (5 mL) was added BF₃.OEt₂ (120 μL, 0.97 mmol). The solution was refluxed for 2 h, evaporated, and the residue was dissolved in pyridine (4 mL) and was treated with acetic anhydride (415 μL, 4.4 mmol). After stirring for 1 h at room temperature the solvent was evaporated and the residue was partitioned between ethyl acetate and 5% citric acid. The organic phase was washed with saturated NaHCO₃, brine, dried (MgSO₄), filtered, and evaporated. The residue was purified by silica gel chromatography (10% methanol in CH₂Cl₂) to afford 823 (76 mg, 29%) as a white solid.

### Example 178

Hydroxy acid 824: A solution of 823 (33 mg, 0.11 mmol) in 2.5M HCl in ethyl acetate (2 mL) was stirred for 2.5 h at room temperature and was evaporated. The residue was dissolved in THF (2 mL) and was treated with 1N KOH (154 μL, 0.16 mmol) and water (300 μL). The reaction was stirred at room temperature for 6 h and was acidified with Dowex 50WX8 ion exchange resin. The resin was filtered and the filtrate was evaporated to afford a residue which was dissolved in water and chromatographed on C₁₈ reverse phase silica gel. After lyophilization, 824 (24 mg, 95%) was isolated as a white solid.

### Example 179

Methyl ether 825: To a solution of 820 (80 mg, 0.17 mmol) in methanol (2 mL) was added BF₃.OEt₂ (32 μL, 0.26 mmol). The solution was refluxed for 2 h, evaporated, and the residue was dissolved in pyridine (2 mL). To the solution was added acetic anhydride (80 μL, 0.85 mmol) and catalytic DMAP. After stirring 14 h, the solvent was evaporated and the residue was chromatographed on silica gel (ethyl acetate) to afford 825 (46 mg, 90%) as a white solid.

### Example 180

Hydroxy acid 826: To a solution of 825 (46 mg, 0.15 mmol) in THF (2 mL) was added 1N KOH (433 μL, 0.45 mmol) and the mixture was stirred at room temperature for 5 h. The solution was acidified with Dowex 50WX8 ion exchange resin and the resin was filtered and washed with methanol. Sovent was evaporated to afford a residue which was dissolved in water and passed through a column of C₁₈ reverse phase silica eluting with water. The solvent was evaporated to give 826 (33 mg, 96%) as a white solid.

### Example 181

Methyl ether 827: To a solution of 816 (612 mg, 0.27 mmol) in methanol (25 mL) was added BF₃.OEt₂ (370 μL, 3.0 mmol). The solution was refluxed for 2 h, evaporated, and the residue was dissolved in CH₂Cl₂ (5 mL) and was treated with di-tert-butyldicarbonate (880 mg, 4.1 mmol) in CH₂Cl₂ (3 mL) and Et₃N (570 μL, 4.1 mmol). After stirring for 5 h at room temperature the solvent was evaporated and the residue was partitioned between ethyl acetate and water. The organic phase was washed with water, brine, dried (MgSO₄), filtered, and evaporated. The residue was purified by silica gel chromatography (2/1-hexane/ethyl acetate) to afford 827 (630 mg, 65%) as an oil.

### Example 182

N-Trityl aziridine 828: A solution of 827 (574 mg, 1.6 mmol) in 2.5 M HCl in ethyl acetate (20 mL) was stirred at room temperature for 5 h. The solvent was evaporated to afford a white solid (400 mg). To a suspension of the solid in CH₂Cl₂ (5 mL) at 0° C. was added trityl chloride (490 mg, 1.6 mmol) and Et₃N (278 μL, 3.6 mmol). The solution was stirred for 2 h at which time Et₃N (278 μL, 3.6 mmol) and methanesulfonyl chloride (136 μL, 1.76 mmol) were added. The reaction mixture was stirred at 0° C. for 1 h and was warmed to room temperature stirring for 4 h. The solvent was evaporated and the residue was partitioned between ether and water. The organic phase was washed with water and the combined aqueous washes were extracted with ether. The combined organic extracts were washed with brine, dried (MgSO₄), filtered and evaporated. Purification of the residue by silica gel chromatography (CH₂Cl₂) afforded 828 (170 mg, 25%) as a white foam.

### Example 183

Bis-methyl ether 829: To a solution of 828 (60 mg, 0.14 mmol) in methanol (2 mL) was added BF₃.OEt₂ (26 μL, 0.21 mmol). The solution was refluxed for 1 h, evaporated, and the residue was dissolved in pyridine (1 mL) and was treated with acetic anhydride (66 μL, 0.70 mmol). After stirring for 18 h at room temperature the solvent was evaporated and the residue was partitioned between ethyl acetate and 1N HCl. The organic phase was washed with saturated NaHCO₃, brine, and was dried (MgSO₄), filtered, and evaporated. The residue was purified by silica gel chromatography (10% methanol in CH₂Cl₂) to afford 829 (13 mg, 34%) as a white solid.

### Example 184

Carboxylic acid 830: To a solution of 829 (13 mg, 0.048 mmol) in THF (1 mL) was added 1N KOH (69 μL, 0.072 mmol) and the mixture was stirred at room temperature for 48 h. The solution was acidified with Dowex 50WX8 ion exchange resin and the resin was filtered and washed with methanol. Sovent was evaporated to afford a residue which was dissolved in water and passed through a column of C₁₈ reverse phase silica to give after lyophilization 830 (8 mg, 68%) as a white solid.

### Example 185

Lactone 900: A solution of quinic acid (20 kg, 104 mol; [α]_{D}−43.7° (c=1.12, water); Merck Index 11th ed., 8071:[α]_{D}−42° to −44° (water)), 2,2-dimethoxypropane (38.0 kg, 365 mol) and p-toluenesulfonic acid monohydrate (0.200 kg, 1.05 mol) in acetone (80 kg) was heated at reflux for two hours. The reaction was quenched by addition of 21% sodium ethoxide in ethanol (0.340 kg, 1.05 mol) and most of the solvent was distilled in vacuo. The residue was partitioned between ethyl acetate (108 kg) and water (30 kg). The aqueous layer was back-extracted with ethyl acetate (13 kg) and the combined organic layers were washed with 5% aqueous sodium bicarbonate (14 kg). Most of the ethyl acetate was distilled in vacuo to leave a pale yellow solid residue of 900 which was used directly in the next step.

### Example 186

Hydroxy ester 901: A solution of the crude lactone 900 (from 104 mol (−)-quinic acid) in absolute ethanol (70 kg) was treated with 20% sodium ethoxide in ethanol (0.340 kg, 1.05 mol). After two hours at room temperature, acetic acid (0.072 kg, 1.2 mol) was added and the solvent was distilled in vacuo. Ethyl acetate (36 kg) was added and the distillation continued to near dryness. The tan solid residue composed of a ca. 5:1 mixture of 901:900 was dissolved in ethyl acetate (9 kg) at reflux and hexane (9 kg) was added. Upon cooling, a white crystalline solid formed which was isolated by filtration to afford a ca. 6.5:1 mixture of 901:900 (19.0 kg, 70% yield).

### Example 187

Mesyl ester 902: A solution of a ca. 6.5:1 mixture (18.7 kg, ca. 72 mol) of hydroxy ester 901 and lactone 900 in dichloromethane (77 kg) was cooled to 0-10° C. and treated with methanesulfonyl chloride (8.23 kg, 71.8 mol), followed by slow addition of triethylamine (10.1 kg, 100 mol). An additional portion of methanesulfonyl chloride (0.84 kg, 7.3 mol) was added. After one hour, water (10 kg) and 3% hydrochloric acid (11 kg) were added. The layers were separated and the organic layer was washed with water (9 kg), then distilled in vacuo to leave a semi-solid residue composed of a ca. 6.5:1 mixture of mesyl ester 902 and mesyl lactone 903. The residue was dissolved in ethyl acetate (11 kg) and cooled to −10° to −20° C. for two hours. Mesyl lactone 903 crystallized and was separated by filtration and washed with cold ethyl acetae (11 kg). The filtrate was concentrated to afford mesyl ester 902 as an orange resin (20.5 kg, 84.3% yield).

### Example 188

Mesyl acetonide 904: A solution of mesyl ester 902 (10.3 kg, 30.4 mol) and pyridine (10.4 kg, 183 mol) in dichloromethane (63 kg) was cooled to −20° to −30° C. and treated portionwise with sulfuryl chloride (6.22 kg, 46 mol). After the exothermic reation subsided, the resulting slurry was quenched with ethanol (2.4 kg), warmed to 0° C., and washed successively with 16% sulfuric acid (35 kg), water (15 kg) and 5% aqueous sodium bicarbonate (1 kg). The organic layer containing a ca. 4:1:1 mixture of 904:905:906 was concentrated in vacuo and ethyl acetate (14 kg) was added. The allylic mesylate 905 was selectively removed by treatment of the ethyl acetate solution with pyrrolidine (2.27 kg, 31.9 mol) and tetrakis(triphenylphosphine)palladium(0) (0.0704 kg, 0.061 mol) at ambient temperature for five hours, followed by washing with 16% sulfuric acid (48 kg). The organic layer was filtered through a pad of silica gel (11 kg) and eluted with ethyl acetate (42 kg). The filtrate was concentrated in vacuo to leave a thick orange oil composed of a ca. 4:1 mixture of 904:906. The residue was dissolved in ethyl acetate (5.3 kg) at reflux and hexane (5.3 kg) was added. Upon cooling, mesyl acetonide 904 crystallized and was separated by filtration and washed with 14% ethyl acetate in hexane (2.1 kg). After drying in vacuo, 904 was obtained as pale yellow needles (4.28 kg, 43.4% yield), mp 102-3° C.

### Example 189

Pentyl ketal 907: A solution of acetonide 904 (8.9 kg, 27.8 mol), 3-pentanone (24 kg, 279 mol) and 70% perchloric acid (0.056 kg, 0.39 mol) was stirred for 18 hours. The volatiles were distilled in vacuo at ambient temperature and fresh 3-pentanone (30 kg, 348 mol) was added gradually as the distillation progressed. The reaction mixture was filtered, toluene (18 kg) was added, and the resulting solution was washed successively with 6% aqueous sodium bicarbonate (19 kg), water (18 kg) and brine (24 kg). The organic layer was concentrated in vacuo and toluene (28 kg) was added gradually as the distillation progressed. When on more distilled, the residual orange oil was composed of pentyl ketal 907 (9.7 kg, 100% yield) and toluene (ca. 2 kg).

### Example 190

Pentyl ether 908: A solution of ketal 907 (8.6 kg, 25 mol) in dichloromethane (90 kg) was cooled to −30° to −20° C. and treated with borane-methyl sulfide complex (2.1 kg, 27.5 mol) and trimethylsilyl trifluoromethanesulfonate (7.2 kg, 32.5 mol). After one hour, 10% aqueous sodium bicarbonate solution (40 kg) was slowly added. The mixture was warmed to ambient temperature and stirred for 12 hours. The organic layer was filtered and concentrated in vacuo to leave a ca. 8:1 mixture of 908:909 as a gray waxy solid (7.8 kg, 90% yield).

### Example 191

Epoxide 910: A ca. 8:1 mixture of isomeric pentyl ethers 908:909 (7.8 kg, 22.3 mol) in ethanol (26 kg) was treated with a solution of potassium hydrogen carbonate (3.52 kg, 35 mol) in water (22 kg). After heating at 55′-65° C. for two hours, the solution was cooled and twice extracted with hexanes (31 kg, then 22 kg). Unreacted 909 remained in the aqueous ethanol layer. The combined hexane extracts were filtered and concentrated in vacuo to leave epoxide 910 as a flocculent white crystalline solid (3.8 kg, 60% yield), mp=54-6° C.

### Example 192

Hydroxy azide 911: A mixture of epoxide 910 (548 g, 2.0 mol), sodium azide (156 g, 2.4 mol) and ammonium chloride (128.4 g, 2.4 mol) in water (0.265 L) and ethanol (1.065 L) was heated at 70°-75° C. for eight hours. Aqueous sodium bicarbonate (0.42 L of 8% solution) was added and the ethanol was distilled in vacuo. The aqueous residue was extracted with ethyl acetate (1 L) and the extract was washed with water (0.5 L). The water wash was back-extracted with ethyl acetate (0.5 L). The combined organic extracts were washed with brine (0.5 L), dried over anhydrous sodium sulfate, filtered and concentrated in vacuo to leave a ca. 10:1 mixture of isomeric hydroxy azides 911:912 (608 g, 102% yield) as a dark brown oil.

### Example 193

Aziridine 913: A ca. 10:1 mixture of hydroxy azides 911:912 (608 g, 2.0 mol) was three times co-evaporated in vacuo from anhydrous acetonitrile (3×0.3 L) and then dissolved in anhydrous acetonitrile (1 L). A solution of anhydrous triphenylphosphine (483 g, 1.84 mol) in anhydrous tetrahydrofuran (0.1 L) and anhydrous acetonitrile (0.92 L) was added dropwise over two hours. The mixture was heated at reflux for six hours then concentrated in vacuo to leave a golden paste composed of aziridine 913, triphenylphosphine oxide and traces of triphenylphosphine. The paste was triturated with diethyl ether (0.35 L). Most of the insoluble triphenylphosphine oxide was removed by filtration and washed with diethyl ether (1.5 L). The filtrate was concentrated in vacuo to leave a dark brown oil which was dissolved in 20% aqueous methanol and extracted three times with hexanes (3×1 L) to remove triphenylphosphine. The hexane extracts were back-extracted with 20% aqueous methanol (0.5 L) and the combined aqueous methanol layers were concentrated in vacuo. The residue was twice co-evaporated in vacuo from anhydrous acetonitrile (2×0.5 L) to leave a dark brown oil composed of aziridene 913 (490 g, 96.8% yield) and triphenylphosphine oxide (ca. 108 g) which was used directly in the next step.

### Example 194

Acetamido azide 915: A mixture of aziridine 913 (490 g, 1.93 mol) and triphenylphosphine oxide (ca. 108 g), sodium azide (151 g, 2.33 mol) and ammonium chloride (125 g, 2.33 mol) in dimethylformamide (1.3 L) was heated at 80°-85° C. for five hours. Sodium bicarbonate (32.8 g, 0.39 mol) and water (0.66 L) were added. The amino azide 914 was isolated from the reaction mixture by six extractions with hexanes (6×1 L). The combined hexane extracts were concentrated in vacuo to ca. 4.5 L total volume and dichloromethane (1.04 L) was added. Aqueous sodium bicarbonate (4.2 L of 8% solution, 3.88 mol) was added, followed by acetic anhydride (198 g, 1.94 mol). After stirring for one hour at ambient temperature, the aqueous layer was discarded. The organic phases were concentrated in vacuo to 1.74 kg total weight and dissolved with ethyl acetate (0.209 L) at reflux. Upon cooling, acetamido azide 915 crystallized and was isolated by filtration. After washing with cold 15% ethyl acetate in hexane (1 L) and drying in vacuo at ambient temperature, pure 915 was obtained as off-white crystals (361 g, 55% yield), mp 126-132° C.

### Example 195

Acetamido amine 916: A mixture of azide 915 (549 g, 1.62 mol) and Lindlar catalyst (50 g) in abs. ethanol (3.25 L) was stirred for eighteen hours while hydrogen (1 atm.) was bubbled through the mixture. Filtration through Celite and concentration of the filtratein vacuo afforded 916 as a foam which solidified on standing (496 g, 98% yield).

### Example 196

Phosphate salt of 916: A solution of acetamido amine 916 (5.02 g, 16.1 mmol) in acetone (75 mL) at reflux was treated with 85% phosphoric acid (1.85 g, 16.1 mmol) in abs. ethanol (25 mL). Crystallization commenced immediately and after cooling to 0° C. for 12 hours the precipitate was collected by filtration to afford 916.H₃PO₄ as long colorless needles (4.94 g, 75% yield; [α]_{D}−39.9° (c=1, water)), mp 203-4° C.

### Example 197

Hydrochloride salt of 916: A solution of acetamido amine 916 (2.8 g, 8.96 mmol) in abs. ethanol (9 mL) was treated with 2.08 M hydrogen chloride in ethanol (8.6 mL, 17.9 mmol). Most of the ethanol was evaporated in vacuo and the oily residue was stirred with ethyl acetate (20 mL) until solid formed. Hexanes (20 mL) were gradually added to the stirred mixture. After one hour at ambient temperature, the solid was collected by filtration, washed with diethyl ether and dried in vacuo. This afforded 916.HCl as an off-white solid (2.54 g, 81% yield; [α]_{D}−43° (c=0.4, water)), mp 206° C.

### Example 198

Aziridine 712: To a solution of azide mesylate 711 (1.27 g, 3.15 mmol) in anhydrous THF (10 mL) at room temperature was added triphenylphosphine (1.0 g, 3.8 mmol) in four portion. The reaction was stirred at room temperature for 3.5 h, then cooled to 0° C., and triethylamine (0.53 mL, 3.8 mmol) and water (0.5 mL) were added. The resulted mixture was stirred at room temperature for 3 h, then at 45° C. for another 3 h. The reaction mixture was evaporated and the residue was partitioned between ethyl acetate and water. The aqueous phase was extracted with ethyl acetate. The combined extracts were washed with brine, dried (MgSO₄), filtered and evaporated. The residue was chromatographed and treated with ethyl ether/hexane (to remove most of the triphenylphosphine oxide) to afford desired aziridine 712 (0.56 g, 65%, with ca. 15% of triphenylphosphine oxide)

### Example 199

N-Acetyl Azide 713: The mixture of aziridine 712 (0.56 g, 17 mmol), sodium azide (0,65 g, 10.0 mmol) and ammonium chloride (0.4 g, 7.5 mmol) in DMF (5.0 mL) was stirred at 65° C. for 18 h. The reaction mixture was diluted with hexane (20 mL) and filtered through a short plug of silica gel (eluted with ethyl acetate/hexane). The filtrate was evaporated. The residue was dissolved in pyridine (5.0 mL), and acetic anhydride (1.0 mL) was added. The resulted mixture was stirred at room temperature for 14 h, and then evaporated. The residue was dissolved in ethyl acetate and washed with saturated NaHCO₃, and brine. The organic phase was dried (MgSO₄), filtered and evaporated. The residue was chromatographed and crystallized from ethyl acetate/hexane to give N-acetyl azide 713 (20 mg, 3.3%) as a solid. ¹H NMR (CDCl₃): 5.68 (d, 1H, J=7.9), 4.31 (d, 1H, J=5.2), 4.09 (m, 1H), 3.94 (m, 1H), 3.83 (s, 3H), 3.65 (m, 1H), 2.82 (ddd, 1H, J=0.9, 5.2, 17.7), 2.55 (ddd, 1H, J=1.5, 7.3, 17.7), 2.06 (s, 3), 1.62 (m, 4H), 0.96 (m, 6H).

All literature and patent citations above are hereby expressly incorporated by reference in their entirety at the locations of their citation. Specifically cited sections or pages of the above cited works are incorporated by reference with specificity. The invention has been described in detail sufficient to allow one of ordinary skill in the art to make and use the subject matter of the following claims. It is apparent that certain modifications of the methods and compositions of the following claims can be made within the scope and spirit of the invention.

What is claimed is:

1. A pharmaceutical formulation comprising an enteric protectant and a compound of the formula: 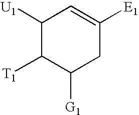
wherein:
E₁ is —CO₂H, —CO₂R₅, —CO₂R₅ₐW₅ or —CO₂W₅;
G₁ is —N₃, —N(R₁₁)₂, —N(R₁₁)C(N(R₁₁))(N(R₁₁)₂), or —C(R₁₁)₂—N(R₁₁)₂;
T₁ is —NH(C(O)CH₃), —NH(C(O)CH₂F), —NH(C(O)CHF₂), or —NH(C(O)CF₃);
U₁ is —OR₄, —SR₄, NHR₄ or N(R₄)₂;
R₁ is independently H or alkyl of 1 to 12 carbon atoms;
R₂ is independently R₃ or R₄ wherein each R₄ is independently substituted with 0 to 3 R₃ groups;
R₃ is independently F, Cl, Br, I, —CN, N₃, —NO₂, OR₆ₐ, —OR₁, —N(R₁)₂, —N(R₁)(R_{6b}), —N(R_{6b})₂, —SR₁, SR₆ₐ, —S(O)R₁, —S(O)₂R₁, —S(O)OR₁, —S(O)OR₆ₐ, —S(O)₂OR₁, —S(O)₂OR₆ₐ, —C(O)OR₁, —C(O)R_{6c}, —C(O)OR₆ₐ, —OC(O)R₁, —N(R₁)(C(O)R₁), —N(R_{6b})(C(O)R₁), —N(R₁)(C(O)OR₁), —N(R_{6b})(C(O)OR₁), —C(O)N(R₁)₂, —C(O)N(R_{6b})(R₁), —C(O)N(R_{6b})₂, —C(NR₁)(N(R₁)₂), —C(N(R_{6b}))(N(R₁)₂), —C(N(R₁))(N(R₁)(R_{6b})), —C(N(R_{6b}))(N(R₁)(R_{6b})), —C(N(R₁))(N(R_{6b})₂), —C(N(R_{6b}))(N(R_{6b})₂), —N(R₁)C(N(R₁))(N(R₁)₂), —N(R₁)C(N(R₁))(N(R₁)(R_{6b})), —N(R₁)C(N(R_{6b}))(N(R₁)₂), —N(R_{6b})C(N(R₁))(N(R₁)₂), —N(R_{6b})C(N(R_{6b}))(N(R₁)₂), —N(R_{6b})C(N(R₁))(N(R₁)(R_{6b})), —N(R₁)C(N(R_{6b}))(N(R₁)(R_{6b})), —N(R₁)C(N(R₁))(N(R_{6b})₂), —N(R_{6b})C(N(R_{6b}))(N(R₁)(R_{6b})), —N(R_{6b})C(N(R₁))(N(R_{6b})₂), —N(R₁)C(N(R_{6b}))(N(R_{6b})₂), —N(R_{6b})C(N(R_{6b}))(N(R_{6b})₂), ═O, ═S, ═N(R₁), or ═N(R_{6b});
R₄ is independently alkyl of 1 to 12 carbon atoms, alkenyl of 2 to 12 carbon atoms, or alkynyl of 2 to 12 carbon atoms; and
R₅ is independently R₄ wherein each R₄ is substituted with 0 to 3 R₃ groups;
R₅ₐ is independently alkylene of 1 to 12 carbon atoms, alkenylene of 2 to 12 carbon atoms, or alkynylene of 2-12 carbon atoms any one of which alkylene, alkenylene or alkynylene is substituted with 0-3 R₃ groups
R₆ₐ is independently H or an ether- or ester-forming group;
R_{6b} is independently H, a protecting group for amino or the residue of a carboxyl-containing compound;
R_{6c} is independently H or the residue of an amino-containing compound;
W₅ is carbocycle or heterocycle wherein W₅ is independently substituted with 0 to 3 R₂ groups; and
R₁₁ is independently H or R₅.

2. The pharmaceutical formulation of claim 1 wherein E₁ is —CO₂R₅; G₁ is NH₂ or N₃; T₁ is NHC(O)CH₃; and U₁ is —OR₄.

3. The pharmaceutical formulation of claim 2 wherein E₁ is C(O)OCH₂CH₃; G₁ is NH₂; T₁ is NHC(O)CH₃; and U₁ is OCH(CH₂CH₃)₂.

4. The pharmaceutical formulation of claim 1 comprising a compound of the formula:

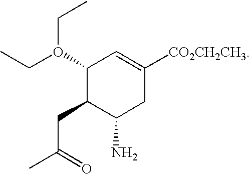

5. The pharmaceutical formulation of claim 4 wherein the compound further comprises a phosphate salt.

6. The pharmaceutical formulation of claim 1 comprising a compound of the formula:

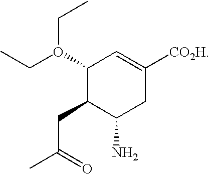

7. The pharmaceutical formulation of claim 1 wherein the enteric protectant is selected from cellulose acetate phthalate polymer, methyl acrylate-methacrylic acid copolymer, cellulose acetate succinate polymer, hydroxypropylmethylcellulose phthalate polymer, polyvinyl acetate phthalate polymer, cellulose acetate trimellitate polymer, hydroxypropyl methylcellulose phthalate succinate polymer, methacrylic acid polymer, and methacrylic acid ester polymer.

8. The pharmaceutical formulation of claim 1 wherein the formulation is a tablet.

9. The pharmaceutical formulation of claim 1 wherein the formulation is a capsule.

10. A pharmaceutical formulation comprising a liquid suspension of enteric coated particles of a compound of claim 1.

11. A method of inhibiting the activity of neuraminidase comprising the step of contacting a sample suspected of containing neuraminidase with a pharmaceutical formulation of claim 1.

12. The method of claim 11 wherein the neuraminidase is influenza neuraminidase in vivo.

13. A method for the treatment or prophylaxis of influenza infection in a host comprising administering to the host a therapeutically effective amount of a pharmaceutical formulation of claim 1.
